SEQUENCE LISTING (1 sequence)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

(19) United States
(12) Patent Application Publication
(10) Pub. No.: US 2026/0132129 A1
(43) Pub. Date: May 14, 2026

(54) 4H-PYRIDO[1,2-A]PYRIMIDIN-4-ONE DERIVATIVES FOR TREATING CANCER

(71) Applicant: Genesis Therapeutics, Inc., Burlingame, CA (US)

(72) Inventors: Brett Busch, San Diego, CA (US); Nicholas Simon Stock, Encinitas, CA (US); Advait Nagle, San Diego, CA (US); Evan Nathaniel Feinberg, Palo Alto, CA (US); Martin Indarte, Krakow (PL)

(21) Appl. No.: 19/118,419

(22) PCT Filed: Oct. 13, 2023
(86) PCT No.: PCT/US2023/076857

§ 371 Date: Apr. 4, 2025

Related U.S. Application Data
(60) Provisional application No. 63/416,311, filed on Oct. 14, 2022.

(51) Int. Cl. C07D 471/04; A61K 31/519; A61K 31/5377; A61K 31/695; A61P 35/00; C07D 519/00; C07F 7/08

(52) U.S. Cl. CPC C07D 471/04; A61K 31/519; A61K 31/5377; A61K 31/695; A61P 35/00; C07D 519/00; C07F 7/0816

(57) ABSTRACT

This disclosure provides compounds of Formula (I) and pharmaceutically acceptable salts thereof, that inhibit phosphatidylinositol 4,5-bisphosphate 3-kinase (PI3K) isoform alpha (PI3Kα). These compounds are useful for treating a disease in which increased PI3Kα activation contributes to the pathology, symptoms, and/or progression of the disease (e.g., cancer) in a subject.

### CROSS-REFERENCE TO RELATED APPLICATIONS

This application claims the benefit of priority to U.S. Application No. 63/416,311, filed on Oct. 14, 2022, the contents of which is hereby incorporated by reference in its entireties.

### SEQUENCE LISTING

This application contains a Sequence Listing that has been submitted electronically as an XML file named “49366-0037WO1_ST26_SL.XML.” The XML file, created on Oct. 13, 2023, is 2,933 bytes in size. The material in the XML file is hereby incorporated by reference in its entirety.

### TECHNICAL FIELD

This disclosure provides compounds of Formula (I) and pharmaceutically acceptable salts thereof, that inhibit phosphatidylinositol 4,5-bisphosphate 3-kinase (PI3K) isoform alpha (PI3Kα). These compounds are useful for treating a disease in which increased PI3Kα activation contributes to the pathology, symptoms, and/or progression of the disease (e.g., cancer) in a subject.

### BACKGROUND

Phosphatidylinositol 4,5-bisphosphate 3-kinase (PI3K) isoform alpha (PI3Kα), encoded by the PIK3CA gene is a part of the PI3K/AKT/TOR signaling network and is altered in several human cancers.

Activation of the PI3K pathway occurs in approximately 30-50% human cancers and contributes to resistance to various anti-cancer therapies. (See, Bauer, T. M. et al., Pharmacol. Ther. 2015, 146, 53-60.) However, development of PI3K inhibitors has been problematic for several reasons, in particular, inability to specifically inhibit signaling by mutant PI3Kα while sparing wild-type PI3Kα, and the related dose-limiting toxicities that prevent sustained PI3K pathway suppression. (See, Hanker et al., Cancer Discovery, April 2019; 9: 482-491.) For example, alpelisib is a PI3K inhibitor that is equipotent against wild-type and mutant forms of PI3Kα, which results in dose-limiting toxicities and hyperglycemia.

Thus, selectively targeting PI3Kα represents an approach for the treatment of proliferative disorders such as cancer.

### SUMMARY

Some embodiments provide a compound of Formula (I):

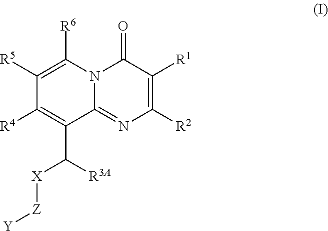

or a pharmaceutically acceptable salt thereof, wherein:
   R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
   R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, C1-C6 alkoxyalkyl optionally substituted with —C(═O)NR^{A}R^{C}, C1-C6 alkoxy optionally substituted with —C(═O)NR^{A}R^{C}, or —O(R^{2B});
   each R^{2A} is independently selected from:
   (i) halogen,
   (ii) cyano,
   (iii) hydroxyl,
   (iv) —NR^{A}R^{B},
   (v) —C(═O)NR^{A}R^{B},

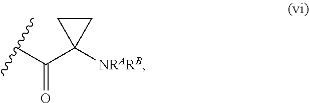

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B} or C1-C6 alkyl optionally substituted with C1-C6 alkoxy or —NHC(═O)C1-C6 alkyl optionally substituted with C3-C16 cycloalkyl,
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
R^{2B} is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A};
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

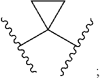 [SMILES: *C1(*)CC1]

Z is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(=NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is hydroxyl, —SO₂(C1-C6 alkyl), or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (I-A):

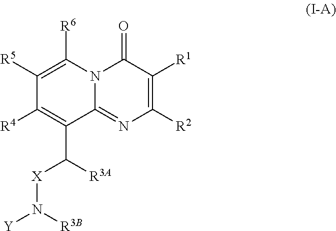

or a pharmaceutically acceptable salt thereof, wherein:
   R¹ is hydrogen, cyano, C3-C6 cycloalkyl, or C1-C6 alkyl optionally substituted with phenyl optionally substituted with halogen;
   R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, or C1-C6 alkoxy optionally substituted with —C(═O)NR^{A}R^{C};
   each R^{2A} is independently selected from:
(i) halogen,
(ii) cyano,
(iii) hydroxyl,
(iv) —NR^{A}R^{B},
(v) —C(═O)NR^{A}R^{B}

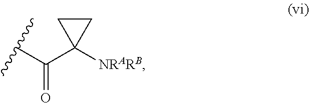

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G} C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
   each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
   each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
   each R^{D} and R^{E} is independently selected from hydrogen, hydroxyl, C1-C6 alkyl, and C1-C6 alkoxy;
   each R^{3A} and R^{3B} is independently selected from hydrogen and C1-C6 alkyl, or R^{3A} and R^{3B}, together with the carbon and nitrogen atoms, respectively, to which they are attached together form a 4-8 membered heterocyclyl group;
   R⁴ is hydrogen, C1-C6 alkyl, or acrylamido;
   R⁵ is hydrogen, C1-C6 alkyl, cyano, —NR^{5A}R^{5B}, —NR^{5A}C(═O)R^{5B}, or —C(═O)NR^{5A}R^{5B};
   R^{5A} and R^{5B} are independently selected from hydrogen, C1-C6 alkyl, C2-C6 alkenyl, and C1-C6 hydroxyalkyl;
   R⁶ is hydrogen, halogen, or C1-C6 alkyl;
   X is a bond, CH₂, CH(CH₃), C(CH₃)₂, or

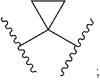 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(=NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(C1-C6 alkyl) or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Also provided herein is a pharmaceutical composition comprising a compound of Formula (I), or a pharmaceutically acceptable salt thereof, and one or more pharmaceutically acceptable excipients.

Provided herein is a method for treating cancer in a subject in need thereof, comprising administering to the subject a therapeutically effective amount of a compound of Formula (I), or a pharmaceutically acceptable salt thereof, or a pharmaceutical composition as provided herein.

Also provided herein is a method for treating cancer in a subject in need thereof, comprising (a) determining that the cancer is associated with a dysregulation of a PIK3CA gene, a PI3Kα protein, or expression or activity or level of any of the same; and (b) administering to the subject a therapeutically effective amount of a compound of Formula (I), or a pharmaceutically acceptable salt thereof, or a pharmaceutical composition as provided herein.

Provided herein is a method of treating a PI3Kα-associated disease in a subject, comprising administering to a subject identified or diagnosed as having a PI3Kα-associated disease a therapeutically effective amount of a compound of Formula (I), or a pharmaceutically acceptable salt thereof, or a pharmaceutical composition as provided herein.

This disclosure also provides a method of treating a PI3Kα-associated disease in a subject, comprising: determining that the cancer in the subject is a PI3Kα-associated disease; and administering to the subject a therapeutically effective amount of a compound of Formula (I), or a pharmaceutically acceptable salt thereof, or a pharmaceutical composition as provided herein.

Further provided herein is a method of treating a PI3Kα-associated cancer in a subject, comprising administering to a subject identified or diagnosed as having a PI3Kα-associated cancer a therapeutically effective amount of a compound of Formula (I), or a pharmaceutically acceptable salt thereof, or a pharmaceutical composition as provided herein.

This disclosure also provides a method of treating a PI3Kα-associated cancer in a subject, comprising: determining that the cancer in the subject is a PI3Kα-associated cancer; and administering to the subject a therapeutically effective amount of a compound of Formula (I), or a pharmaceutically acceptable salt thereof, or a pharmaceutical composition as provided herein.

Provided herein is a method of treating a subject, comprising administering a therapeutically effective amount of a compound of Formula (I), or a pharmaceutically acceptable salt thereof, or a pharmaceutical composition as provided herein, to a subject having a clinical record that indicates that the subject has a dysregulation of a PIK3CA gene, a PI3Kα protein, or expression or activity or level of any of the same.

This disclosure also provides a method for inhibiting PI3Kα in a mammalian cell, comprising contacting the mammalian cell with an effective amount of a compound of Formula (I), or a pharmaceutically acceptable salt thereof.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition).

The phrase “therapeutically effective amount” means an amount of compound that, when administered to a subject in need of such treatment, is sufficient to (i) treat a PI3Kα protein-associated disease, (ii) attenuate, ameliorate, or eliminate one or more symptoms of the particular disease, or (iii) delay the onset of one or more symptoms of the particular disease described herein.

The term “pharmaceutically acceptable salt” refers to a formulation of a compound that does not cause significant irritation to an organism to which it is administered and does not abrogate the biological activity and properties of the compound.

As used herein, the term “subject” refer to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the disease to be treated and/or prevented.

As used herein, terms “treat” or “treatment” refer to therapeutic or palliative measures. Beneficial or desired clinical results include, but are not limited to, alleviation, in whole or in part, of symptoms associated with a disease, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “halogen” refers to fluoro (F), chloro (Cl), bromo (Br), or iodo (I).

The term “oxo” refers to a divalent doubly bonded oxygen atom (i.e., “=O”). As used herein, oxo groups are attached to carbon atoms to form carbonyls.

The term “hydroxyl” refers to an —OH radical.

The term “cyano” refers to a —CN radical.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halogen.

The term “alkoxy” refers to an —O-alkyl radical (e.g., —OCH₃).

The term “alkoxyalkyl” refers to an -alkyl-O-alkyl radical (e.g., —CH₂CH₂OCH₃).

The term “thioalkyl” refers to an —S-alkyl radical (e.g., —SCH₃) or an -alkyl-S-alkyl radical (e.g., —CH₂CH₂SCH₃).

The term “hydroxyalkyl” refers to an alkyl, in which one or more hydrogen atoms is/are replaced with hydroxyl.

The term “aryl” refers to a 6-20 membered all carbon ring system wherein at least one ring in the system is aromatic (e.g., 6-carbon monocyclic, 10-carbon bicyclic, or 14-carbon tricyclic aromatic ring system). Examples of aryl groups include phenyl, naphthyl, tetrahydronaphthyl, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “heteroaryl”, as used herein, refers to a ring system having 5 to 20 ring atoms, such as 5, 6, 9, 10, or 14 ring atoms; wherein at least one ring in the system contains one or more heteroatoms independently selected from the group consisting of N, O, S, Si, and B, and at least one ring in the system is aromatic (but does not have to be a ring which contains a heteroatom, e.g. tetrahydroisoquinolinyl, e.g., tetrahydroquinolinyl). Heteroaryl groups can include monocyclic, bridged, fused, and spiro ring systems, so long as one ring in the system is aromatic. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone (e.g.,

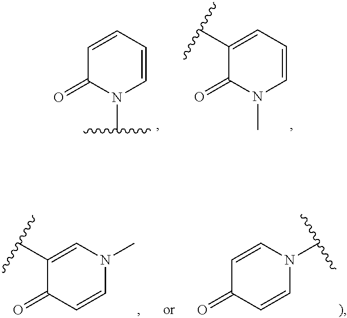

pyrimidone (e.g.,

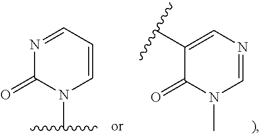

pyridazinone (e.g.,

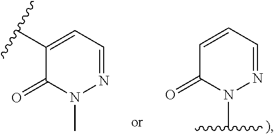

pyrazinone (e.g.,

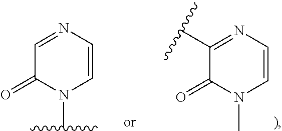

and imidazolone (e.g.,

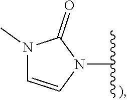 [SMILES: *n1ccn(C)c1=O]

wherein each ring nitrogen adjacent to a carbonyl is tertiary (i.e., the oxo group (i.e., “=O”) herein is a constituent part of the heteroaryl ring).

The term “heterocyclyl” refers to a saturated or partially unsaturated ring systems with 3-16 ring atoms (e.g., 3-8 membered monocyclic, 5-12 membered bicyclic, or 10-14 membered tricyclic ring system) having at least one heteroatom selected from O, N, S, Si, and B, wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting. Heterocyclyl groups include monocyclic, bridged, fused, and spiro ring systems. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

An “aralkyl” refers to an aryl group, as defined herein, connected to the remainder of the molecule via a divalent C1-C6 alkyl group, as described herein. Non-limiting examples of an aralkyl group are benzyl, ethylphenyl, methylnaphthyl, and the like.

A “heteroaralkyl” refers to a heteroaryl group, as defined herein, connected to the remainder of the molecule via a divalent C1-C6 alkyl group, as described herein. Non-limiting examples of an aralkyl group are methylpyridyl, ethylpyrimidinyl, methylimidazolyl, and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself; e.g., one or more double or tirple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge (e.g.,

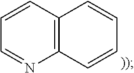

(ii) a single ring atom (spiro-fused ring systems) (e.g.,

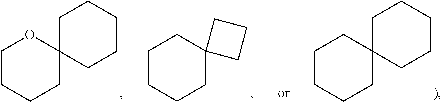

or (iii) a contiguous array of ring atoms (bridged ring systems having all bridge lengths >0) (e.g.,

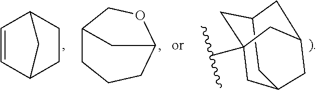

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

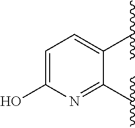 [SMILES: *c1ccc(O)nc1*]

encompasses the tautomeric form containing the moiety:

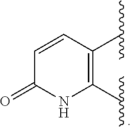 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and drawings, and from the claims.

### DETAILED DESCRIPTION

This disclosure provides compounds of Formula (I) and pharmaceutically acceptable salts thereof, that inhibit phosphatidylinositol 4,5-bisphosphate 3-kinase (PI3K) isoform alpha (PI3Kα). These compounds are useful for treating a disease in which increased PI3Kα activation contributes to the pathology, symptoms, and/or progression of the disease (e.g., cancer) in a subject.

### Formulae (I) Compounds

Some embodiments provide a compound of Formula (I):

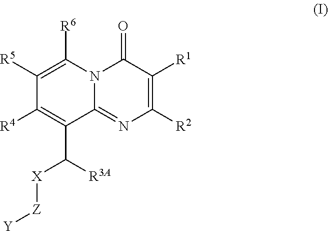

or a pharmaceutically acceptable salt thereof, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, C1-C6 alkoxyalkyl optionally substituted with —C(═O)NR^{A}R^{C}, C1-C6 alkoxy optionally substituted with —C(═O)NR^{A}R^{C}, or —O(R^{2B});
each R^{2A} is independently selected from:
(i) halogen,
(ii) cyano,
(iii) hydroxyl,
(iv) —NR^{A}R^{B},
(v) —C(═O)NR^{A}R^{B},

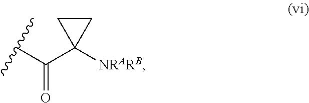

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B} or C1-C6 alkyl optionally substituted with C1-C6 alkoxy or —NHC(═O)C1-C6 alkyl optionally substituted with C3-C16 cycloalkyl
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
R^{2B} is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A};
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

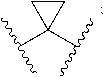 [SMILES: *C1(*)CC1]

Z is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(=NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (I-A):

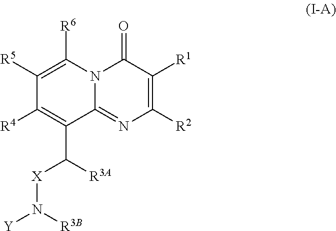

or a pharmaceutically acceptable salt thereof, wherein:
   R¹ is hydrogen, cyano, C3-C6 cycloalkyl, or C1-C6 alkyl optionally substituted with phenyl optionally substituted with halogen;
   R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, or C1-C6 alkoxy optionally substituted with —C(═O)NR^{A}R^{C};
   each R^{2A} is independently selected from:
(i) halogen,
(ii) cyano,
(iii) hydroxyl,
(iv) —NR^{A}R^{B},
(v) —C(═O)NR^{A}R^{B},

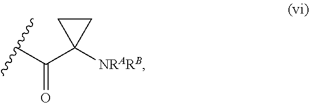

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G} C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
   each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
   each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
   each R^{D} and R^{E} is independently selected from hydrogen, hydroxyl, C1-C6 alkyl, and C1-C6 alkoxy;
   each R^{3A} and R^{3B} is independently selected from hydrogen and C1-C6 alkyl, or R^{3A} and R^{3B}, together with the carbon and nitrogen atoms, respectively, to which they are attached together form a 4-8 membered heterocyclyl group;
   R⁴ is hydrogen, C1-C6 alkyl, or acrylamido;
   R⁵ is hydrogen, C1-C6 alkyl, cyano, —NR^{5A}R^{5B}, —NR^{5A}C(═O)R^{5B}, or —C(═O)NR^{5A}R^{5B};
   R^{5A} and R^{5B} are independently selected from hydrogen, C1-C6 alkyl, C2-C6 alkenyl, and C1-C6 hydroxyalkyl;
   R⁶ is hydrogen, halogen, or C1-C6 alkyl;
   X is a bond, CH₂, CH(CH₃), C(CH₃)₂, or

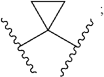 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(=NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(C1-C6 alkyl) or C1-C6 alkyl optionally substituted with oxo; and
each R^{F} and R^{G} is independently selected from hydrogen, phenyl, and C1-C6 alkyl optionally substituted with oxo or —NR^{A}R^{B}.

In some embodiments, R¹ is hydrogen.

In some embodiments, R¹ is cyano.

In some embodiments, R¹ is C3-C6 cycloalkyl. In some embodiments, R¹ is cyclopropyl or cyclobutyl.

In some embodiments, R¹ is C1-C6 alkyl optionally substituted with phenyl optionally substituted with halogen. In some embodiments, R¹ is C1-C6 alkyl substituted with phenyl optionally substituted with halogen. In some embodiments, R¹ is C1-C6 alkyl substituted with phenyl substituted with halogen. In some embodiments, R¹ is para-fluorobenzyl. In some embodiments, R¹ is C1-C6 alkyl substituted with phenyl. In some embodiments, R¹ is benzyl. In some embodiments, R¹ is ethyl-1-phenyl or ethyl-2-phenyl. In some embodiments, R¹ is C1-C6 alkyl. In some embodiments, R¹ is methyl, ethyl, or isopropyl. In some embodiments, R¹ is methyl.

In some embodiments, R¹ is C1-C6 thioalkyl. In some embodiments, R¹ is C1-C3 thioalkyl. In some embodiments, R¹ is thiomethyl, thioethyl, or thiopropyl. In some embodiments, R¹ is methyl-thiomethyl, methyl-thioethyl, or ethyl-thiomethyl. In some embodiments, R¹ is thiomethyl.

In some embodiments, R¹ is C1-C6 haloalkyl. In some embodiments, R¹ is C1-C3 haloalkyl. In some embodiments, R¹ is C1-C3 fluoroalkyl. In some embodiments, R¹ is CF₃. In some embodiments, R¹ is CHF₂.

In some embodiments, R¹ is C1-C6 alkoxy. In some embodiments, R¹ is C1-C3 alkoxy. In some embodiments, R¹ is —OCH₃, —OCH₂CH₃, or —OCH₂CH₂CH₃. In some embodiments, R¹ is —OCH₃.

In some embodiments, R¹ is C1-C6 alkoxyalkyl. In some embodiments, R¹ is C1-C3 alkoxyalkyl. In some embodiments, R¹ is —CH₂OCH₃, —CH₂OCH₂CH₃, or —CH₂CH₂OCH₃. In some embodiments, R¹ is —CH₂OCH₃.

In some embodiments, R² is phenyl optionally substituted with 1-4 independently selected R^{2A}. In some embodiments, R² is phenyl substituted with 1 or 2 independently selected R^{2A}. In some embodiments, R² is phenyl substituted with 1 R^{2A}. In some embodiments, R² is phenyl substituted with 2 independently selected R^{2A}. In some embodiments, R² is phenyl optionally substituted with 3 independently selected R^{2A}. In some embodiments, R² is phenyl.

In some embodiments, R² is 5-10 membered heteroaryl optionally substituted with 1-4 independently selected R^{2A}. In some embodiments, R² is 5-10 membered heteroaryl substituted with 1 or 2 independently selected R^{2A}. In some embodiments, R² is 5-10 membered heteroaryl substituted with 1 R^{2A}. In some embodiments, R² is 5-10 membered heteroaryl substituted with 2 independently selected R^{2A}. In some embodiments, R² is 5-10 membered heteroaryl optionally substituted with 3 independently selected R^{2A}. In some embodiments, R² is 5-10 membered heteroaryl.

In some embodiments, R² is 6 membered heteroaryl optionally substituted with 1-4 independently selected R^{2A}. In some embodiments, R² is 6 membered heteroaryl substituted with 1 or 2 independently selected R^{2A}. In some embodiments, R² is 6 membered heteroaryl substituted with 1 R^{2A}. In some embodiments, R² is 6 membered heteroaryl substituted with 2 independently selected R^{2A}. In some embodiments, R² is 6 membered heteroaryl optionally substituted with 3 independently selected R^{2A}. In some embodiments, R² is 6 membered heteroaryl.

In some embodiments, R² is 9 membered heteroaryl optionally substituted with 1-4 independently selected R^{2A}. In some embodiments, R² is 9 membered heteroaryl substituted with 1 or 2 independently selected R^{2A}. In some embodiments, R² is 9 membered heteroaryl substituted with 1 R^{2A}. In some embodiments, R² is 9 membered heteroaryl substituted with 2 independently selected R^{2A}. In some embodiments, R² is 9 membered heteroaryl optionally substituted with 3 independently selected R^{2A}. In some embodiments, R² is 9 membered heteroaryl.

In some embodiments, the heteroaryl of R² is pyridinyl, pyrimidinyl, pyridazinyl, indole, indazole, azaindole, azaindazole, indoline, azaindoline, isoindoline, azaisoindoline, benzimidazolyl, benzoxazolyl, benzothiazolyl, benzoisoxazolyl, benzisothiazolyl, quinolinyl, or isoquinolinyl. In some embodiments, the heteroaryl of R² is pyridinyl or pyrimidinyl. In some embodiments, the heteroaryl of R² is indole, indazole, azaindole, azaindazole, indoline, azaindoline, isoindoline, or azaisoindoline.

In some embodiments, R² is 4-10 membered heterocyclyl optionally substituted with 1-4 independently selected R^{2A}. In some embodiments, R² is 4-10 membered heterocyclyl substituted with 1 or 2 independently selected R^{2A}. In some embodiments, R² is 4-10 membered heterocyclyl substituted with 1 R^{2A}. In some embodiments, R² is 4-10 membered heterocyclyl substituted with 2 independently selected R^{2A}. In some embodiments, R² is 4-10 membered heterocyclyl optionally substituted with 3 independently selected R^{2A}. In some embodiments, R² is 4-10 membered heterocyclyl.

In some embodiments, R² is 5-8 membered heterocyclyl optionally substituted with 1-4 independently selected R^{2A}. In some embodiments, R² is 5-8 membered heterocyclyl substituted with 1 or 2 independently selected R^{2A}. In some embodiments, R² is 5-8 membered heterocyclyl substituted with 1 R^{2A}. In some embodiments, R² is 5-8 membered heterocyclyl substituted with 2 independently selected R^{2A}. In some embodiments, R² is 5-8 membered heterocyclyl optionally substituted with 3 independently selected R^{2A}. In some embodiments, R² is 5-8 membered heterocyclyl.

In some embodiments, the heterocyclyl of R² is piperidinyl, piperazinyl, or morpholinyl.

In some embodiments, R² is 4-10 membered cycloalkyl optionally substituted with 1-4 independently selected R^{2A}. In some embodiments, R² is 4-10 membered cycloalkyl substituted with 1 or 2 independently selected R^{2A}. In some embodiments, R² is 4-10 membered cycloalkyl substituted with 1 R^{2A}. In some embodiments, R² is 4-10 membered cycloalkyl substituted with 2 independently selected R^{2A}. In some embodiments, R² is 4-10 membered cycloalkyl optionally substituted with 3 independently selected R^{2A}. In some embodiments, R² is 4-10 membered cycloalkyl.

In some embodiments, R² is 5-7 membered cycloalkyl optionally substituted with 1-4 independently selected R^{2A}. In some embodiments, R² is 5-7 membered cycloalkyl substituted with 1 or 2 independently selected R^{2A}. In some embodiments, R² is 5-7 membered cycloalkyl substituted with 1 R^{2A}. In some embodiments, R² is 5-7 membered cycloalkyl substituted with 2 independently selected R^{2A}. In some embodiments, R² is 5-7 membered cycloalkyl optionally substituted with 3 independently selected R^{2A}. In some embodiments, R² is 5-7 membered cycloalkyl.

In some embodiments, the cycloalkyl of R² is cyclopentyl, [1.1.1]bicyclopentyl, or cyclohexyl.

In some embodiments, R² is C1-C6 alkoxyalkyl optionally substituted with —C(═O)NR^{A}R^{C}. In some embodiments, R² is C1-C6 alkoxyalkyl. In some embodiments, R² is C1-C3 alkoxyalkyl. In some embodiments, R² is —CH₂OCH₃, —CH₂OCH₂CH₃, or —CH₂CH₂OCH₃. In some embodiments, R² is —CH₂OCH₃. In some embodiments, 1, 2, 3, or 4 of R^{2A} are independently halogen. In some embodiments, 1, 2, or 3 of R^{2A} are independently fluoro or chloro. In some embodiments, 1 or 2 of R^{2A} are independently fluoro or chloro.

In some embodiments, 1, 2, 3, or 4 of R^{2A} are independently cyano. In some embodiments, 1 or 2 of R^{2A} are cyano.

In some embodiments, 1, 2, 3, or 4 of R^{2A} are independently hydroxyl. In some embodiments, 1 or 2 of R^{2A} are hydroxyl.

In some embodiments, 1, 2, 3, or 4 of R^{2A} are independently —NR^{A}R^{B} In some embodiments, 1 or 2 of R^{2A} are independently —NR^{A}R^{B}.

In some embodiments, 1, 2, 3, or 4 of R^{2A} are independently —C(═O)NR^{A}R^{B}. In some embodiments, 1 or 2 of R^{2A} are independently —C(═O)NR^{A}R^{B}.

In some embodiments, 1, 2, 3, or 4 of R^{2A} are independently

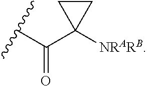

In some embodiments, 1 or 2 of R^{2A} are independently

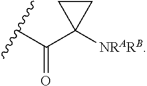

In some embodiments, 1, 2, 3, or 4 of R^{2A} are independently —NHC(═O)R^{C}. In some embodiments, 1 or 2 of R^{2A} are independently —NHC(═O)R^{C}.

In some embodiments, 1, 2, 3, or 4 of R^{2A} are independently —C(═O)NR^{D}R^{E}. In some embodiments, 1 or 2 of R^{2A} are independently —C(═O)NR^{D}R^{E}.

In some embodiments, 1, 2, 3, or 4 of R^{2A} are independently —C(═O)OR^{F}. In some embodiments, 1 or 2 of R^{2A} are independently —C(═O)OR^{F}.

In some embodiments, 1, 2, 3, or 4 of R^{2A} are independently —SO₂R^{F}. In some embodiments, 1 or 2 of R^{2A} are independently —SO₂R^{F}.

In some embodiments, 1, 2, 3, or 4 of R^{2A} are independently —NHSO₂R^{F}. In some embodiments, 1 or 2 of R^{2A} are independently —NHSO₂R^{F}.

In some embodiments, 1, 2, 3, or 4 of R^{2A} are independently —SO₂NR^{F}R^{G}. In some embodiments, 1 or 2 of R^{2A} are independently —SO₂NR^{F}R^{G}.

In some embodiments, 1, 2, 3, or 4 of R^{2A} are independently —NHC(═O)C1-C6 alkyl optionally substituted with NR^{A}R^{B}. In some embodiments, 1, 2, or 3 of R^{2A} are independently —NHC(═O)C1-C6 alkyl substituted with NR^{A}R^{B}. In some embodiments, 1, 2, or 3 of R^{2A} are independently —NHC(═O)C1-C6 alkyl. In some embodiments, 1 or 2 of R^{2A} are independently —NHC(═O)C1-C6 alkyl optionally substituted with NR^{A}R^{B}. In some embodiments, 1 or 2 of R^{2A} are independently —NHC(═O)C1-C6 alkyl substituted with NR^{A}R^{B}. In some embodiments, 1 or 2 of R^{2A} are independently —NHC(═O)C1-C6 alkyl.

In some embodiments, 1, 2, 3, or 4 of R^{2A} are independently C1-C6 haloalkyl. In some embodiments, 1 or 2 of R^{2A} are independently C1-C3 haloalkyl. In some embodiments, 1 or 2 of R^{2A} are trifluoromethyl.

In some embodiments, 1, 2, 3, or 4 of R^{2A} are independently C1-C6 hydroxyalkyl. In some embodiments, 1 or 2 of R^{2A} are independently C1-C3 hydroxyalkyl.

In some embodiments, 1, 2, 3, or 4 of R^{2A} are independently 5-10 membered heteroaryl optionally substituted with 1-3 substituents independently selected from C1-C6 alkyl and —NR^{A}R^{B}. In some embodiments, 1, 2, 3, or 4 of R^{2A} are independently 5-10 membered heteroaryl substituted with 1-3 substituents independently selected from C1-C6 alkyl and —NR^{A}R^{B} In some embodiments, 1, 2, 3, or 4 of R^{2A} are independently 5-10 membered heteroaryl.

In some embodiments, 1 of R^{2A} is 5-6 membered heteroaryl optionally substituted with 1-3 substituents independently selected from C1-C6 alkyl and —NR^{A}R^{B}. In some embodiments, 1 of R^{2A} is 5-6 membered heteroaryl substituted with 1-3 substituents independently selected from C1-C6 alkyl and —NR^{A}R^{B}. In some embodiments, 1 of R^{2A} is 5-6 membered heteroaryl.

In some embodiments, 1, 2, 3, or 4 of R^{2A} are independently 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B}, or C1-C6 alkyl optionally substituted with C1-C6 alkoxy or —NHC(═O)C1-C6 alkyl optionally substituted with C3-C16 cycloalkyl. In some embodiments, 1 of R^{2A} is 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B}, or C1-C6 alkyl optionally substituted with C1-C6 alkoxy or —NHC(═O)C1-C6 alkyl optionally substituted with C3-C16 cycloalkyl.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B}, or C1-C6 alkyl optionally substituted with C1-C6 alkoxy or —NHC(═O)C1-C6 alkyl optionally substituted with C3-C16 cycloalkyl. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B}, or C1-C6 alkyl optionally substituted with C1-C6 alkoxy or —NHC(═O)C1-C6 alkyl optionally substituted with C3-C16 cycloalkyl.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl substituted with C1-C6 alkoxy, —C(═O)NR^{A}R^{B}, or —NHC(═O)C1-C6 alkyl substituted with —NR^{A}R^{B}. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl substituted with C1-C6 alkoxy, —C(═O)NR^{A}R^{B}, or —NHC(═O)C1-C6 alkyl substituted with —NR^{A}R^{B}.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl, —C(═O)NR^{A}R^{B}, or —NHC(═O)C1-C6 alkyl. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl, —C(═O)NR^{A}R^{B}, or —NHC(═O)C1-C6 alkyl.

In some embodiments, 1, 2, 3, or 4 of R^{2A} are independently 4-10 membered heterocyclyl substituted with C1-C6 alkyl optionally substituted with C1-C6 alkoxy or —NHC(═O)C1-C6 alkyl optionally substituted with C3-C16 cycloalkyl. In some embodiments, R^{2A} is C1-C6 alkyl substituted with C1-C6 alkoxy or —NHC(═O)C1-C6 alkyl optionally substituted with C3-C16 cycloalkyl.

In some embodiments, 1, 2, 3, or 4 of R^{2A} are independently 4-10 membered heterocyclyl. In some embodiments, 1 of R^{2A} is 4-10 membered heterocyclyl.

In some embodiments, 1, 2, or 3 of R^{2A} are independently C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl. In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, and 4-10 membered heterocyclyl substituted with hydroxyl, C1-C6 alkyl, aralkyl, heteroaralkyl, —C(═O)NR^{A}R^{B}, or —C(═O)C3-C6 cycloalkyl.

In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, and 4-10 membered heterocyclyl.

In some embodiments, 1, 2, 3, or 4 of R^{2A} are independently C1-C6 alkyl. In some embodiments, 1, 2, or 3 of R^{2A} are independently C1-C3 alkyl. In some embodiments, 1, 2, or 3 of R^{2A} are methyl. In some embodiments, 1 or 2 of R^{2A} are independently C1-C6 alkyl. In some embodiments, 1 or 2 of R^{2A} are independently C1-C3 alkyl. In some embodiments, 1 or 2 of R^{2A} are methyl.

In some embodiments, 1, 2, 3, or 4 of R^{2A} are independently C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl optionally substituted with C1-C6 alkyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl substituted with C1-C6 alkyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl.

In some embodiments, 1, 2, 3, or 4 of R^{2A} are independently C1-C6 alkoxy.

In some embodiments, 1, 2, 3, or 4 of R^{2A} are independently C3-C6 cycloalkyl optionally substituted with 4-10 membered heterocyclyl optionally substituted with C1-C6 alkyl.

In some embodiments, 1, 2, 3, or 4 of R^{2A} are independently C3-C6 cycloalkyl substituted with 4-10 membered heterocyclyl optionally substituted with C1-C6 alkyl.

In some embodiments, 1, 2, 3, or 4 of R^{2A} are independently C3-C6 cycloalkyl substituted with 4-10 membered heterocyclyl substituted with C1-C6 alkyl.

In some embodiments, 1, 2, 3, or 4 of R^{2A} are independently C3-C6 cycloalkyl substituted with 4-10 membered heterocyclyl.

In some embodiments, 1, 2, 3, or 4 of R^{2A} are independently C3-C6 cycloalkyl. In some embodiments, 1 or 2 of R^{2A} are independently C3-C6 cycloalkyl.

In some embodiments, R² is —O(R^{2B}).

In some embodiments, R^{2B} is phenyl optionally substituted with 1-4 independently selected R^{2A}. In some embodiments, R^{2B} is phenyl substituted with 1 or 2 independently selected R^{2A}. In some embodiments, R^{2B} is phenyl substituted with 1 R^{2A}. In some embodiments, R^{2B} is phenyl substituted with 2 independently selected R^{2A}. In some embodiments, R^{2B} is phenyl optionally substituted with 3 independently selected R^{2A}. In some embodiments, R^{2B} is phenyl.

In some embodiments, R^{2B} is 5-10 membered heteroaryl optionally substituted with 1-4 independently selected R^{2A}. In some embodiments, R^{2B} is 5-10 membered heteroaryl substituted with 1 or 2 independently selected R^{2A}. In some embodiments, R^{2B} is 5-10 membered heteroaryl substituted with 1 R^{2A}. In some embodiments, R^{2B} is 5-10 membered heteroaryl substituted with 2 independently selected R^{2A}. In some embodiments, R^{2B} is 5-10 membered heteroaryl optionally substituted with 3 independently selected R^{2A}. In some embodiments, R^{2B} is 5-10 membered heteroaryl.

In some embodiments, the heteroaryl of R^{2B} is pyridinyl, pyrimidinyl, pyridazinyl, indole, indazole, azaindole, azaindazole, indoline, azaindoline, isoindoline, azaisoindoline, benzimidazolyl, benzoxazolyl, benzothiazolyl, benzoisoxazolyl, benzisothiazolyl, quinolinyl, or isoquinolinyl. In some embodiments, the heteroaryl of R^{2B} is pyridinyl or pyrimidinyl. In some embodiments, the heteroaryl of R^{2B} is indole, indazole, azaindole, azaindazole, indoline, azaindoline, isoindoline, or azaisoindoline.

In some embodiments, R^{2B} is 4-10 membered heterocyclyl optionally substituted with 1-4 independently selected R^{2A}. In some embodiments, R^{2B} is 4-10 membered heterocyclyl substituted with 1 or 2 independently selected R^{2A}. In some embodiments, R^{2B} is 4-10 membered heterocyclyl substituted with 1 R^{2A}. In some embodiments, R^{2B} is 4-10 membered heterocyclyl substituted with 2 independently selected R^{2A}. In some embodiments, R^{2B} is 4-10 membered heterocyclyl optionally substituted with 3 independently selected R^{2A}. In some embodiments, R^{2B} is 4-10 membered heterocyclyl.

In some embodiments, R^{2B} is 5-8 membered heterocyclyl optionally substituted with 1-4 independently selected R^{2A}. In some embodiments, R^{2B} is 5-8 membered heterocyclyl substituted with 1 or 2 independently selected R^{2A}. In some embodiments, R^{2B} is 5-8 membered heterocyclyl substituted with 1 R^{2A}. In some embodiments, R^{2B} is 5-8 membered heterocyclyl substituted with 2 independently selected R^{2A}. In some embodiments, R^{2B} is 5-8 membered heterocyclyl optionally substituted with 3 independently selected R^{2A}. In some embodiments, R^{2B} is 5-8 membered heterocyclyl.

In some embodiments, the heterocyclyl of R^{2B} is piperidinyl, piperazinyl, tetrahydropyranyl, or morpholinyl.

In some embodiments, R^{2B} is 4-10 membered cycloalkyl optionally substituted with 1-4 independently selected R^{2A}. In some embodiments, R^{2B} is 4-10 membered cycloalkyl substituted with 1 or 2 independently selected R^{2A}. In some embodiments, R^{2B} is 4-10 membered cycloalkyl substituted with 1 R^{2A}. In some embodiments, R^{2B} is 4-10 membered cycloalkyl substituted with 2 independently selected R^{2A}. In some embodiments, R^{2B} is 4-10 membered cycloalkyl optionally substituted with 3 independently selected R^{2A}. In some embodiments, R^{2B} is 4-10 membered cycloalkyl.

In some embodiments, the cycloalkyl of R^{2B} is cyclopentyl, [1.1.1]bicyclopentyl, or cyclohexyl.

In some embodiments, R² is piperidinyl substituted with 1-2 independently selected R^{2A}. In some embodiments, R² is piperidinyl substituted with 2-4 independently selected R^{2A}. In some embodiments, R² is morpholinyl substituted with 2-4 independently selected R^{2A}. In some embodiments, R² is morpholinyl. In some embodiments, R² is piperazinyl substituted with 1-2 independently selected R^{2A}.

In some embodiments, R² is phenyl substituted with 1-2 independently selected R^{2A}.

In some embodiments, R² is indolyl substituted with 1-2 independently selected R^{2A}. In some embodiments, R² is indolyl substituted with 2 independently selected R^{2A}, where at least one R^{2A} is C1-C6 alkyl. In some embodiments, R² is indolyl substituted with 2 independently selected R^{2A}, where one R^{2A} is C1-C6 alkyl and one R^{2A} is 4-10 membered heterocyclyl.

In some embodiments, R² is indazolyl substituted with 1-2 independently selected R^{2A}. In some embodiments, R² is 7-azaindolyl substituted with 1-2 independently selected R^{2A}. In some embodiments, R² is 7-azaindazolyl substituted with 1-2 independently selected R^{2A}. In some embodiments, R² is 1,2-dihydro-3H-indazol-3-one substituted with 1-2 independently selected R^{2A}. In some embodiments, R² is isoindolinyl substituted with 1-2 independently selected R^{2A}. In some embodiments, R² is isoindolinyl. In some embodiments, R² is 2-indolinone substituted with 1-2 independently selected R^{2A}. In some embodiments, R² is beznimidazolyl substituted with 1-2 independently selected R^{2A}. In some embodiments, R² is imidazopyridinyl substituted with 1-2 independently selected R^{2A}. In some embodiments, R² is 1,3-dihydro-2H-benzo[d]imidazol-2-onyl substituted with 1-2 independently selected R^{2A}. In some embodiments, R² is [1,2,4]triazolo[1,5-a]pyridine substituted with 1-2 independently selected R^{2A}.

In some embodiments, one R^{2A} is C1-C6 alkyl, C1-C6 haloalkyl, C1-C6 hydroxyalkyl, cyano, or hydroxyl, and the other R^{2A} are independently selected from C1-C6 haloalkyl, C1-C6 hydroxyalkyl, cyano, hydroxyl, halogen, —NR^{A}R^{B}, —C(═O)NR^{A}R^{B},

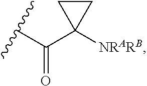

—NHC(═O)R^{C}, —C(═O)NR^{D}R^{E}, —C(═O)OR^{F}, —SO₂R^{F}, —NHSO₂R^{F}, —SO₂NR^{F}R^{G}, —NHC(═O)C1-C6 alkyl optionally substituted with NR^{A}R^{B}, 5-10 membered heteroaryl optionally substituted with 1-3 substituents independently selected from C1-C6 alkyl and —NR^{A}R^{B}, 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}, C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl, C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and C3-C6 cycloalkyl optionally substituted with 4-10 membered heterocyclyl optionally substituted with C1-C6 alkyl.

In some embodiments, each R^{2A} is independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, 1, 2, or 3 R^{2A} are independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, 1 or 2 R^{2A} are independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, R² is substituted with 1 R^{2A}. In some embodiments, R² is substituted with 2 independently selected R^{2A}. In some embodiments, R² is substituted with 3 independently selected R^{2A}. In some embodiments, R² is substituted with 4 independently selected R^{2A}.

In some embodiments, R² is substituted with 1 R^{2A}. In some embodiments, R^{2A} is halogen. In some embodiments, R^{2A} is fluoro or chloro. In some embodiments, R^{2A} is cyano. In some embodiments, R^{2A} is hydroxyl. In some embodiments, R^{2A} is R^{2A}—NR^{A}R^{B}. In some embodiments, R^{2A} is —C(═O)NR^{A}R^{B}. In some embodiments, R^{2A} is

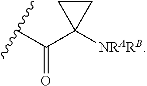

In some embodiments, R^{2A} is —NHC(═O)R^{C}. In some embodiments, —C(═O)NR^{D}R^{E}. In some embodiments, R^{2A} is —C(═O)OR^{F}. In some embodiments, R^{2A} is —SO₂R^{F}. In some embodiments, R^{2A} is —NHSO₂R^{F}. In some embodiments, R^{2A} is —NHC(═O)C1-C6 alkyl optionally substituted with NR^{A}R^{B}. In some embodiments, R^{2A} is C1-C6 haloalkyl. In some embodiments, R^{2A} is C1-C6 hydroxyalkyl. In some embodiments, R^{2A} is 5-10 membered heteroaryl optionally substituted with 1-3 substituents independently selected from C1-C6 alkyl and —NR^{A}R^{B}. In some embodiments, R^{2A} is 5-6 membered heteroaryl optionally substituted with 1-3 substituents independently selected from C1-C6 alkyl and —NR^{A}R^{B}. In some embodiments, R^{2A} is 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}. In some embodiments, R^{2A} is 4-10 membered heterocyclyl. In some embodiments, R^{2A} is C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl. In some embodiments, R^{2A} is C1-C6 alkyl. In some embodiments, R^{2A} is C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl. In some embodiments, R^{2A} is C1-C6 alkoxy. In some embodiments, R^{2A} is C3-C6 cycloalkyl optionally substituted with 4-10 membered heterocyclyl optionally substituted with C1-C6 alkyl. In some embodiments, R^{2A} is selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, R² is C1-C6 alkoxy optionally substituted with —C(═O)NR^{A}R^{C}. In some embodiments, R² is C1-C6 alkoxy substituted with —C(═O)NR^{A}R^{C}. In some embodiments, R² is C3-C6 alkoxy substituted with —C(═O)NR^{A}R^{C}. In some embodiments, R² is C1-C6 alkoxy.

In some embodiments, R² is C1-C6 alkylalkoxy optionally substituted with —C(═O)NR^{A}R^{C}. In some embodiments, R² is C1-C6 alkylalkoxy substituted with —C(═O)NR^{A}R^{C}. In some embodiments, R² is C3-C6 alkylalkoxy substituted with —C(═O)NR^{A}R^{C}. In some embodiments, R² is C1-C6 alkylalkoxy.

In some embodiments, X is a bond.

In some embodiments, X is CH₂.

In some embodiments, X is CH(CH₃).

In some embodiments, X is C(CH₃)₂.

In some embodiments, X is

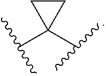 [SMILES: *C1(*)CC1]

In some embodiments, Z is O.

In some embodiments, R^{3A} is hydrogen.

In some embodiments, R^{3A} is C1-C6 alkyl. In some embodiments, R^{3A} is methyl or ethyl. In some embodiments, R^{3A} is methyl.

In some embodiments, R^{3A} is C1-C6 alkoxy. In some embodiments, R^{3A} is C1-C3 alkoxy. In some embodiments, R^{3A} is —OCH₃, —OCH₂CH₃, or —OCH₂CH₂CH₃. In some embodiments, R^{3A} is —OCH₃.

In some embodiments, R^{3A} is C1-C6 haloalkyl. In some embodiments, R^{3A} is C1-C3 haloalkyl. In some embodiments, R^{3A} is C1-C3 fluoroalkyl. In some embodiments, R^{3A} is CF₃. In some embodiments, R^{3A} is CHF₂.

In some embodiments, Z is NR^{3B}.

In some embodiments, one of R^{3A} and R^{3B} is hydrogen and the other of R^{3A} and R^{3B} is C1-C6 alkyl. In some embodiments, one of R^{3A} and R^{3B} is hydrogen and the other of R^{3A} and R^{3B} is methyl. In some embodiments, each of R^{3A} and R^{3B} is hydrogen. In some embodiments, each of R^{3A} and R^{3B} is an independently selected C1-C6 alkyl. In some embodiments, each of R^{3A} and R^{3B} is methyl.

In some embodiments, one of R^{3A} and R^{3B} is hydrogen and the other of R^{3A} and R^{3B} is C1-C6 alkoxy. In some embodiments, one of R^{3A} and R^{3B} is C1-C6 alkyl and the other of R^{3A} and R^{3B} is C1-C6 alkoxy. In some embodiments, R^{3A} is C1-C6 alkoxy. In some embodiments, R^{3A} is C1-C3 alkoxy. In some embodiments, R^{3A} is —OCH₃, —OCH₂CH₃, or —OCH₂CH₂CH₃. In some embodiments, R^{3A} is —OCH₃.

In some embodiments, one of R^{3A} and R^{3B} is hydrogen and the other of R^{3A} and R^{3B} is C1-C6 haloalkyl. In some embodiments, one of R^{3A} and R^{3B} is C1-C6 alkyl and the other of R^{3A} and R^{3B} is C1-C6 haloalkyl. In some embodiments, R^{3A} is C1-C6 haloalkyl. In some embodiments, R^{3A} is C1-C3 haloalkyl. In some embodiments, R^{3A} is C1-C3 fluoroalkyl. In some embodiments, R^{3A} is CF₃. In some embodiments, R^{3A} is CHF₂.

In some embodiments, R^{3A} and R^{3B}, together with the carbon and nitrogen atoms, respectively, to which they are attached together form a 4-8 membered heterocyclyl group. In some embodiments, R^{3A} and R^{3B}, together with the carbon and nitrogen atoms, respectively, to which they are attached together form a 5-6 membered heterocyclyl group.

In some embodiments, Y is phenyl optionally substituted with R^{Y}, naphthyl substituted with R^{Y}, or 5-10 membered heteroaryl substituted with R^{Y}.

In some embodiments, Y is phenyl optionally substituted with 1-3 independently selected R^{Y}. In some embodiments, Y is phenyl substituted with 1 or 2 independently selected R^{Y}. In some embodiments, Y is phenyl substituted with 1 R^{Y}. In some embodiments, Y is phenyl substituted with 2 independently selected R^{Y}. In some embodiments, Y is phenyl optionally substituted with 3 independently selected R^{Y}. In some embodiments, Y is phenyl.

In some embodiments, Y is naphthyl optionally substituted with 1-3 independently selected R^{Y}. In some embodiments, Y is naphthyl substituted with 1 or 2 independently selected R^{Y}. In some embodiments, Y is naphthyl substituted with 1 R^{Y}. In some embodiments, Y is naphthyl substituted with 2 independently selected R^{Y}. In some embodiments, Y is naphthyl optionally substituted with 3 independently selected R^{Y}. In some embodiments, Y is naphthyl.

In some embodiments, Y is 5-10 membered heteroaryl optionally substituted with 1-3 independently selected R^{Y}. In some embodiments, Y is 5-10 membered heteroaryl substituted with 1 or 2 independently selected R^{Y}. In some embodiments, Y is 5-10 membered heteroaryl substituted with 1 R^{Y}. In some embodiments, Y is 5-10 membered heteroaryl substituted with 2 independently selected R^{Y}. In some embodiments, Y is 5-10 membered heteroaryl optionally substituted with 3 independently selected R^{Y}. In some embodiments, Y is 5-10 membered heteroaryl.

In some embodiments, Y is 6 membered heteroaryl optionally substituted with 1-3 independently selected R^{Y}. In some embodiments, Y is 6 membered heteroaryl substituted with 1 or 2 independently selected R^{Y}. In some embodiments, Y is 6 membered heteroaryl substituted with 1 R^{Y}. In some embodiments, Y is 6 membered heteroaryl substituted with 2 independently selected R^{Y}. In some embodiments, Y is 6 membered heteroaryl optionally substituted with 3 independently selected R^{Y}. In some embodiments, Y is 6 membered heteroaryl.

In some embodiments, Y is 9 membered heteroaryl optionally substituted with 1-3 independently selected R^{Y}. In some embodiments, Y is 9 membered heteroaryl substituted with 1 or 2 independently selected R^{Y}. In some embodiments, Y is 9 membered heteroaryl substituted with 1 R^{Y}. In some embodiments, Y is 9 membered heteroaryl substituted with 2 independently selected R^{Y}. In some embodiments, Y is 9 membered heteroaryl optionally substituted with 3 independently selected R^{Y}. In some embodiments, Y is 9 membered heteroaryl.

In some embodiments, 1, 2, or 3 of R^{Y} is independently halogen. In some embodiments, 1, 2, or 3 of R^{Y} is independently chloro or fluoro. In some embodiments, 1 or 2 of R^{Y} is independently chloro or fluoro.

In some embodiments, 1, 2, or 3 of R^{Y} is hydroxyl. In some embodiments, 1 or 2 of R^{Y} is hydroxyl.

In some embodiments, 1, 2, or 3 of R^{Y} is cyano. In some embodiments, 1 or 2 of R^{Y} is cyano.

In some embodiments, 1, 2, or 3 of R^{Y} is independently C1-C6 haloalkyl optionally substituted with hydroxyl. In some embodiments, 1, 2, or 3 of R^{Y} is independently C1-C6 haloalkyl substituted with hydroxyl. In some embodiments, 1, 2, or 3 of R^{Y} is independently C1-C6 haloalkyl. In some embodiments, 1 or 2 of R^{Y} is independently C1-C3 haloalkyl. In some embodiments, 1 or 2 of R^{Y} is trifluoromethyl.

In some embodiments, 1, 2, or 3 of R^{Y} is independently C1-C6 alkoxy. In some embodiments, 1 or 2 of R^{Y} is independently C1-C3 alkoxy. In some embodiments, 1 or 2 of R^{Y} is methoxy.

In some embodiments, 1, 2, or 3 of R^{Y} is independently C1-C6 haloalkoxy. In some embodiments, 1 or 2 of R^{Y} is independently C1-C3 haloalkoxy. In some embodiments, 1 or 2 of R^{Y} is trifluoromethoxy.

In some embodiments, 1, 2, or 3 of R^{Y} is independently C1-C6 hydroxyalkyl. In some embodiments, 1 or 2 of R^{Y} is independently C1-C3 hydroxyalkyl. In some embodiments, 1 or 2 of R^{Y} is independently mono-hydroxyl C1-C3 alkyl. In some embodiments, 1 or 2 of R^{Y} is independently di-hydroxyl C2-C3 alkyl.

In some embodiments, 1, 2, or 3 of R^{Y} is independently —NHC(═O)R^{C}. In some embodiments, 1 or 2 of R^{Y} is independently —NHC(═O)R^{C}. In some embodiments, 1 of R^{Y} is —NHC(═O)R^{C}. In some embodiments, Y is substituted with 1 R^{Y}, and R^{Y} is —NHC(═O)R^{C}. In some embodiments, 1, 2, or 3 of R^{Y} is independently —C(═O)NHR^{Y1}. In some embodiments, 1 or 2 of R^{Y} is independently —C(═O)NR^{Y1}. In some embodiments, 1 of R^{Y} is —C(═O)NHR^{Y1}. In some embodiments, Y is substituted with 1 R^{Y}, and R^{Y} is —C(═O)NHR^{Y1}.

In some embodiments, 1, 2, or 3 of R^{Y} is independently —CO₂R^{A}. In some embodiments, 1 or 2 of R^{Y} is independently —CO₂R^{A}. In some embodiments, 1 of R^{Y} is —CO₂R^{A}. In some embodiments, Y is substituted with 1 R^{Y}, and R^{Y} is —CO₂R^{A}. In some embodiments, Y is substituted with 1 R^{Y}, and R^{Y} is —CO₂H.

In some embodiments, 1, 2, or 3 of R^{Y} is independently —SO₂NR^{F}R^{G}. In some embodiments, 1 or 2 of R^{Y} is independently —SO₂NR^{F}R^{G}. In some embodiments, 1 of R^{Y} is —SO₂NR^{F}R^{G}. In some embodiments, Y is substituted with 1 R, and R^{Y} is —SO₂NR^{F}R^{G}.

In some embodiments, 1, 2, or 3 of R^{Y} is independently —NHSO₂R^{F}. In some embodiments, 1 or 2 of R^{Y} is independently —NHSO₂R^{F}. In some embodiments, 1 of R^{Y} is —NHSO₂R^{F}. In some embodiments, Y is substituted with 1 R^{Y}, and R^{Y} is —NHSO₂R^{F}.

In some embodiments, 1, 2, or 3 of R^{Y} is independently —S(═O)(=NR^{F})R^{G}. In some embodiments, 1 or 2 of R^{Y} is independently —S(═O)(=NR^{F})R^{G}. In some embodiments, 1 of R^{Y} is —S(═O)(=NR^{F})R^{G}. In some embodiments, Y is substituted with 1 R^{Y}, and R^{Y} is —S(═O)(=NR^{F})R^{G}.

In some embodiments, 1, 2, or 3 of R^{Y} is independently —SO₂(C1-C6 alkyl). In some embodiments, 1 or 2 of R^{Y} is independently —SO₂(C1-C6 alkyl). In some embodiments, 1 of R is —SO₂(C1-C6 alkyl). In some embodiments, 1 or 2 of R^{Y} is —SO₂CH₃. In some embodiments, 1 of R^{Y} is —SO₂CH₃. In some embodiments, Y is substituted with 1 R^{Y}, and R^{Y} is —SO₂CH₃.

In some embodiments, 1, 2, or 3 of R^{Y} is independently —C(═O)NR^{A}R^{B}. In some embodiments, 1 or 2 of R^{Y} is independently —C(═O)NR^{A}R^{B}. In some embodiments, 1 of R^{Y} is —C(═O)NR^{A}R^{B}. In some embodiments, Y is substituted with 1 R^{Y}, and R^{Y} is —C(═O)NR^{A}R^{B}.

In some embodiments, 1, 2, or 3 of R^{Y} is independently 4-6 membered heteroaryl. In some embodiments, 1 of R¹ is 4-6 membered heteroaryl. In some embodiments, Y is substituted with 1 R^{Y}, and R^{Y} is 4-6 membered heteroaryl.

In some embodiments, 1, 2, or 3 of R^{Y} is independently heteroaralkyl. In some embodiments, 1 of R^{Y} is independently heteroaralkyl.

In some embodiments, 1, 2, or 3 of R^{Y} is independently 4-6 membered heterocyclyl optionally substituted with R^{Y1}. In some embodiments, 1 of R^{Y} is 4-6 membered heterocyclyl. In some embodiments, 1 of R^{Y} is 4-6 membered heterocyclyl substituted with R^{Y1}. In some embodiments, Y is substituted with 1 R^{Y}, and R^{Y} is 4-6 membered heterocyclyl substituted with R^{Y1}, where R^{Y1} is hydroxyl. In some embodiments, R^{Y} is

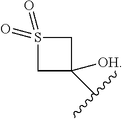 [SMILES: *C1(O)CS(=O)(=O)C1]

In some embodiments, R^{Y} is

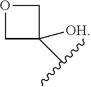 [SMILES: *C1(O)COC1]

In some embodiments, Y is selected from the group consisting of:

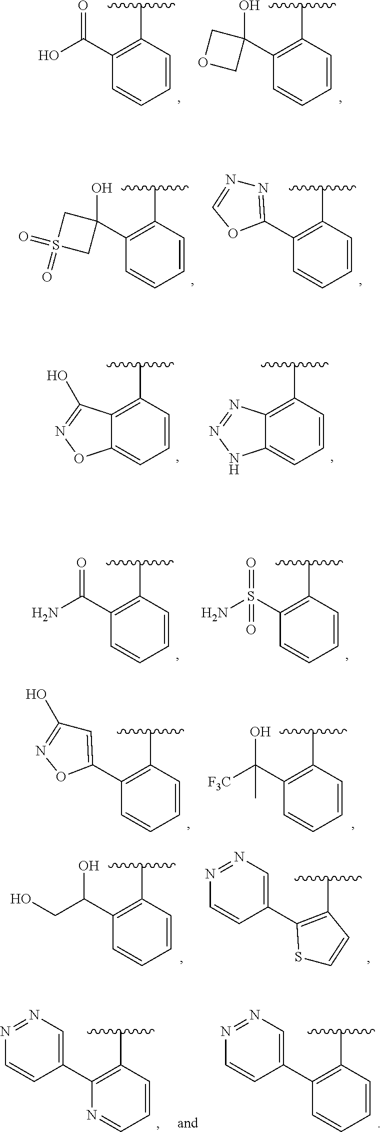

In some embodiments, 1, 2, or 3 of R^{Y} is independently C1-C6 alkyl optionally substituted with —CO₂R^{A} or 4-6 membered heteroaryl optionally substituted with R^{Y1}. In some embodiments, 1, 2, or 3 of R^{Y} is independently C1-C6 alkyl substituted with —CO₂R^{A} or 4-6 membered heteroaryl optionally substituted with R^{Y1}. In some embodiments, 1, 2, or 3 of R^{Y} is independently C1-C6 alkyl substituted with —CO₂R^{A} or 4-6 membered heteroaryl substituted with R^{Y1}. In some embodiments, 1, 2, or 3 of R^{Y} is independently C1-C6 alkyl substituted with —CO₂R^{A} or 4-6 membered heteroaryl. In some embodiments, Y is substituted with 1 R^{Y}, and R^{Y} is C1-C6 alkyl substituted with —CO₂R^{A}. In some embodiments, Y is substituted with 1 R^{Y}, and R^{Y} is C1-C6 alkyl substituted with —CO₂H.

In some embodiments, 1 or 2 of R^{Y} is independently C1-C6 alkyl optionally substituted with —CO₂R^{A} or 4-6 membered heteroaryl optionally substituted with R^{Y1}. In some embodiments, 1 or 2 of R^{Y} is independently C1-C6 alkyl substituted with —CO₂R^{A} or 4-6 membered heteroaryl optionally substituted with R^{Y1}. In some embodiments, 1 or 2 of R^{Y} is independently C1-C6 alkyl substituted with —CO₂R^{A} or 4-6 membered heteroaryl substituted with R^{Y1}. In some embodiments, 1 or 2 of R^{Y} is independently C1-C6 alkyl substituted with —CO₂R^{A} or 4-6 membered heteroaryl.

In some embodiments, 1 or 2 of R^{Y} is independently C1-C6 alkyl substituted with —CO₂R^{A}. In some embodiments, 1 or 2 of R^{Y} is independently C1-C6 alkyl substituted 4-6 membered heteroaryl optionally substituted with R^{Y1}. In some embodiments, 1 or 2 of R^{Y} is independently C1-C6 alkyl substituted with 4-6 membered heteroaryl substituted with R^{Y1}. In some embodiments, 1 or 2 of R^{Y} is independently C1-C6 alkyl substituted with 4-6 membered heteroaryl.

In some embodiments, Y is substituted with 1 R^{Y}. In some embodiments, Y is substituted with 2 independently selected R^{Y}. In some embodiments, Y is substituted with 3 independently selected R^{Y}.

In some embodiments, Y is substituted with 1 R^{Y}. In some embodiments, R^{Y} is halogen. In some embodiments, R^{Y} is chloro or fluoro. In some embodiments, R^{Y} is hydroxyl. In some embodiments, R^{Y} is cyano. In some embodiments, R^{Y} is C1-C6 haloalkyl optionally substituted with hydroxyl. In some embodiments, R^{Y} is C1-C6 haloalkyl. In some embodiments, R^{Y} is C1-C6 alkoxy. In some embodiments, R^{Y} is C1-C6 haloalkoxy. In some embodiments, R^{Y} is C1-C6 hydroxyalkyl. In some embodiments, R^{Y} is —NHC(═O)R^{C}. In some embodiments, R^{Y} is —C(═O)NHR^{Y1}. In some embodiments, R^{Y} is —CO₂R^{A}. In some embodiments, R^{Y} is —SO₂NR^{F}R^{G}. In some embodiments, R^{Y} is —NHSO₂R^{F}. In some embodiments, R^{Y} is —S(═O)(=NR^{F})R^{G}. In some embodiments, R^{Y} is —SO₂(C1-C6 alkyl). In some embodiments, R^{Y} is —C(═O)NR^{A}R^{B}. In some embodiments, R^{Y} is 4-6 membered heteroaryl. In some embodiments, R^{Y} is heteroaralkyl. In some embodiments, R^{Y} is 4-6 membered heterocyclyl optionally substituted with R^{Y1}. In some embodiments, R^{Y} is C1-C6 alkyl optionally substituted with —CO₂R^{A} or 4-6 membered heteroaryl optionally substituted with R^{Y1}.

In some embodiments, 1, 2, or 3 of R^{Y} is independently C1-C6 alkyl. In some embodiments, 1 or 2 of R^{Y} is independently C1-C3 alkyl. In some embodiments, 1, 2, or 3 of R is methyl.

In some embodiments, R^{Y1} is —SO₂(C1-C6 alkyl). In some embodiments, R^{Y1} is —SO₂CH₃.

In some embodiments, R^{Y1} is C1-C6 alkyl optionally substituted with oxo. In some embodiments, R^{Y1} is C1-C6 alkyl substituted with oxo. In some embodiments, R^{Y1} is acetyl, 1-oxoethyl, or 1-oxopropyl. In some embodiments, R^{Y1} is C1-C6 alkyl. In some embodiments, R^{Y1} is methyl.

In some embodiments, R^{Y1} is hydroxyl.

In some embodiments, R⁴ is hydrogen.

In some embodiments, R⁴ is C1-C6 alkyl. In some embodiments, R⁴ is methyl or ethyl. In some embodiments, R⁴ is methyl.

In some embodiments, R⁴ is acrylamido.

In some embodiments, R⁵ is hydrogen.

In some embodiments, R⁵ is C1-C6 alkyl. In some embodiments, R⁵ is methyl or ethyl. In some embodiments, R⁵ is methyl.

In some embodiments, R⁵ is cyano.

In some embodiments, R⁵ is —NR^{5A}R^{5B}.

In some embodiments, R⁵ is —NR^{5A}C(═O)R^{5B}.

In some embodiments, R⁵ is —C(═O)NR^{5A}R^{5B}.

In some embodiments, one of R^{5A} and R^{5B} is hydrogen and the other of R^{5A} and R^{5B} is C1-C6 alkyl, C2-C6 alkenyl, or C1-C6 hydroxyalkyl. In some embodiments, one of R^{5A} and R^{5B} is C1-C6 alkyl and the other of R^{5A} and R^{5B} is C1-C6 alkyl, C2-C6 alkenyl, or C1-C6 hydroxyalkyl. In some embodiments, each of R^{5A} and R^{5B} is hydrogen. In some embodiments, each of R^{5A} and R^{5B} is an independently selected C1-C6 alkyl. In some embodiments, each of R^{5A} and R^{5B} is methyl. In some embodiments, the C1-C6 hydroxyalkyl of R^{5A} and R^{5B} is hydroxymethyl, 1-hydroxyethyl, 2-hydroxyethyl, dihydroxypropyl or dihydroxybutyl.

In some embodiments, R⁵ is acrylamido.

In some embodiments, R⁶ is hydrogen.

In some embodiments, R⁶ is halogen. In some embodiments, R⁶ is fluoro. In some embodiments, R⁶ is chloro.

In some embodiments, R⁶ is C1-C6 alkyl. In some embodiments, R⁶ is methyl.

In some embodiments, each of R^{A} and R^{B} are independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl optionally substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl.

In some embodiments, one of R^{A} and R^{B} is hydrogen and the other of R^{A} and R^{B} is hydroxyl. In some embodiments, one of R^{A} and R^{B} is hydrogen and the other of R^{A} and R^{B} is C1-C6 alkoxy. In some embodiments, one of R^{A} and R^{B} is hydrogen and the other of R^{A} and R^{B} is C3-C6 cycloalkyl. In some embodiments, one of R^{A} and R^{B} is hydrogen and the other of R^{A} and R^{B} is C2-C6 alkenyl. In some embodiments, one of R^{A} and R^{B} is hydrogen and the other of R^{A} and R^{B} is C1-C6 alkyl optionally substituted with hydroxyl or C1-C6 alkoxy. In some embodiments, one of R^{A} and R^{B} is hydrogen and the other of R^{A} and R^{B} is C1-C6 alkyl substituted with hydroxyl. In some embodiments, one of R^{A} and R^{B} is hydrogen and the other of R^{A} and R^{B} is C1-C6 alkyl substituted with C1-C6 alkoxy.

In some embodiments, one of R^{A} and R^{B} is hydrogen and the other of R^{A} and R^{B} is C1-C6 alkyl. In some embodiments, each of R^{A} and R^{B} are hydrogen. In some embodiments, each of R^{A} and R^{B} are an independently selected C1-C6 alkyl optionally substituted with hydroxyl or C1-C6 alkoxy. In some embodiments, each of R^{A} and R^{B} are an independently selected C1-C6 alkyl. In some embodiments, each of R^{A} and R^{B} are methyl.

In some embodiments, R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl.

In some embodiments, R^{A} and R^{B} together with the nitrogen atom to which they are attached form a 4-10 membered heterocyclyl substituted with 1-2 substituents independently selected from halogen, C1-C6 alkyl, and —C(═O)C1-C6 alkyl.

In some embodiments, R^{A} and R^{B} together with the nitrogen atom to which they are attached form a 4-10 membered heterocyclyl substituted with halogen, C1-C6 alkyl, or —C(═O)C1-C6 alkyl. In some embodiments, R^{A} and R^{B} together with the nitrogen atom to which they are attached form a 4-10 membered heterocyclyl substituted with 1-2 substitutents independently selected from fluoro, methyl, and acetyl.

In some embodiments, R^{A} and R^{B} together with the nitrogen atom to which they are attached form a 4-10 membered heterocyclyl. In some embodiments, R^{A} and R^{B} together with the nitrogen atom to which they are attached form a 4-6 membered heterocyclyl.

In some embodiments, each R^{C} is independently C3-C6 cycloalkyl, —C(═O)NHR^{Y1}, or a C1-C6 alkyl substituted with —NR^{A}R^{B} or with 4-10 membered heterocyclyl optionally substituted with C1-C6 alkyl or with C1-C6 hydroxylalkyl. In some embodiments, each R^{C} is independently C3-C6 cycloalkyl, —C(═O)NHR^{Y1}, or a C1-C6 alkyl substituted with —NR^{A}R^{B} or with 4-10 membered heterocyclyl substituted with C1-C6 alkyl or with C1-C6 hydroxylalkyl. In some embodiments, each R^{C} is independently C3-C6 cycloalkyl, —C(═O)NHR^{Y1}, or a C1-C6 alkyl substituted with —NR^{A}R^{B} or with 4-10 membered heterocyclyl. In some embodiments, each R^{C} is independently C3-C6 cycloalkyl, —C(═O)NHR^{Y1}, or a C1-C6 alkyl.

In some embodiments, each R^{C} is independently C3-C6 cycloalkyl. In some embodiments, each R^{C} is independently —C(═O)NHR^{Y1}. In some embodiments, each R^{C} is independently C1-C6 alkyl optionally substituted with —NR^{A}R^{B} or with 4-10 membered heterocyclyl optionally substituted with C1-C6 alkyl or with C1-C6 hydroxylalkyl. In some embodiments, each R^{C} is independently C1-C6 alkyl substituted with —NR^{A}R^{B} or with 4-10 membered heterocyclyl optionally substituted with C1-C6 alkyl or with C1-C6 hydroxylalkyl.

In some embodiments, one of R^{D} and R^{E} is hydrogen and the other of R^{D} and R^{E} is hydroxyl, C1-C6 alkyl, or C1-C6 alkoxy. In some embodiments, one of R^{D} and R^{E} is hydrogen and the other of R^{D} and R^{E} is hydroxyl. In some embodiments, one of R^{D} and R^{E} is hydrogen and the other of R^{D} and R^{E} is C1-C6 alkyl. In some embodiments, one of R^{D} and R^{E} is hydrogen and the other of R^{D} and R^{E} is C1-C6 alkoxy. In some embodiments, one of R^{D} and R^{E} is hydrogen and the other of R^{D} and R^{E} is hydroxyl, methyl, or methoxy. In some embodiments, each of R^{D} and R^{E} is hydrogen. In some embodiments, each of R^{D} and R^{E} is an independently selected C1-C6 alkyl. In some embodiments, each of R^{D} and R^{E} is methyl.

In some embodiments, one of R^{F} and R^{G} is hydrogen and the other of R^{F} and R^{G} is phenyl or C1-C6 alkyl optionally substituted with oxo or —NR^{A}R^{B}. In some embodiments, one of R^{E} and R^{G} is hydrogen and the other of R^{F} and R^{G} is phenyl or C1-C6 alkyl substituted with oxo or —NR^{A}R^{B}. In some embodiments, one of R^{F} and R^{G} is hydrogen and the other of R^{F} and R^{G} is phenyl or C1-C6 alkyl. In some embodiments, one of R^{F} and R^{G} is hydrogen and the other of R^{F} and R^{G} is phenyl. In some embodiments, one of R^{F} and R^{G} is hydrogen and the other of R^{F} and R^{G} is C1-C6 alkyl optionally substituted with oxo or —NR^{A}R^{B}. In some embodiments, one of R^{F} and R^{G} is hydrogen and the other of R^{F} and R^{G} is C1-C6 alkyl substituted with oxo or —NR^{A}R^{B} In some embodiments, each of R^{F} and R^{G} is hydrogen. In some embodiments, each of R^{F} and R^{G} is an independently selected C1-C6 alkyl. In some embodiments, each of R^{E} and R^{G} is methyl.

In some embodiments, the compound of Formula (I), or a pharmaceutically acceptable salt thereof, is a compound of Formula (I-A1):

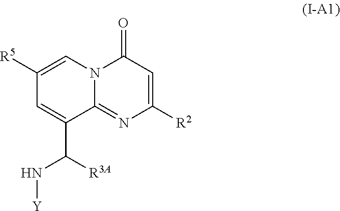

or a pharmaceutically acceptable salt thereof.

In some embodiments, the compound of Formula (I), or a pharmaceutically acceptable salt thereof, is a compound of Formula (I-AA):

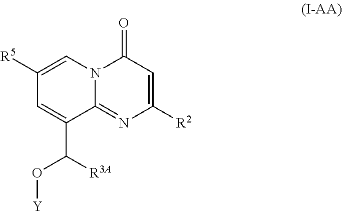

In some embodiments, the compound of Formula (I), or a pharmaceutically acceptable salt thereof, is a compound of Formula (I-B):

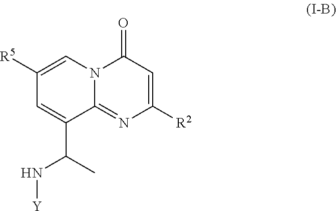

or a pharmaceutically acceptable salt thereof.

In some embodiments, the compound of Formula (I), or a pharmaceutically acceptable salt thereof, is a compound of Formula (I-C):

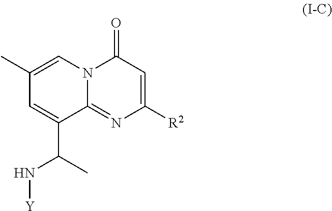

or a pharmaceutically acceptable salt thereof.

In some embodiments, the compound of Formula (I), or a pharmaceutically acceptable salt thereof, is a compound of Formula (I-D):

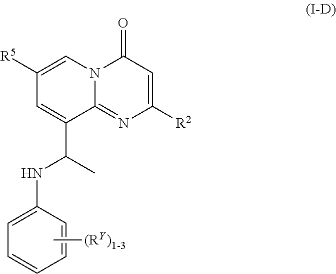

or a pharmaceutically acceptable salt thereof.

In some embodiments, the compound of Formula (I), or a pharmaceutically acceptable salt thereof, is a compound of Formula (I-E):

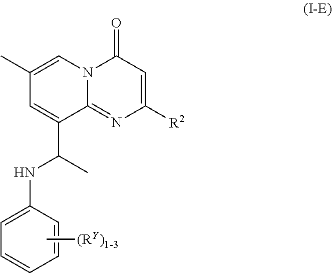

or a pharmaceutically acceptable salt thereof.

In some embodiments, the compound of Formula (I), or a pharmaceutically acceptable salt thereof, is a compound of Formula (I-F):

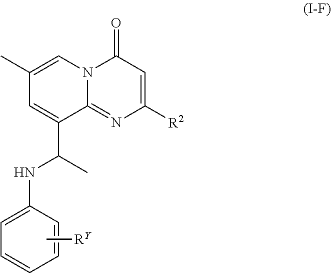

or a pharmaceutically acceptable salt thereof.

In some embodiments, the compound of Formula (I), or a pharmaceutically acceptable salt thereof, is a compound of Formula (I-FF):

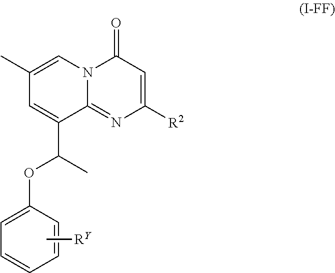

or a pharmaceutically acceptable salt thereof.

In some embodiments, the compound of Formula (I), or a pharmaceutically acceptable salt thereof, is a compound of Formula (I-G):

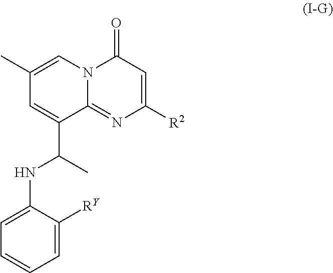

or a pharmaceutically acceptable salt thereof.

In some embodiments, the compound of Formula (I), or a pharmaceutically acceptable salt thereof, is a compound of Formula (I-G1):

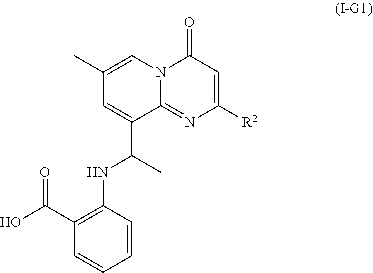

or a pharmaceutically acceptable salt thereof.

In some embodiments, the compound of Formula (I), or a pharmaceutically acceptable salt thereof, is a compound of Formula (I-G2):

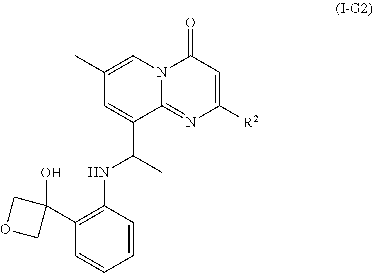

or a pharmaceutically acceptable salt thereof.

In some embodiments, the compound of Formula (I), or a pharmaceutically acceptable salt thereof, is a compound of Formula (I-G3):

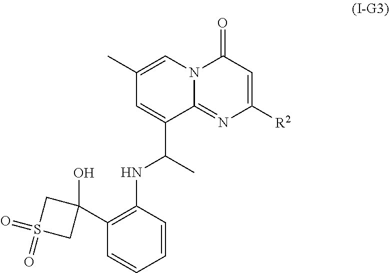

or a pharmaceutically acceptable salt thereof.

In some embodiments, the compound of Formula (I), or a pharmaceutically acceptable salt thereof, is a compound of Formula (I-G4):

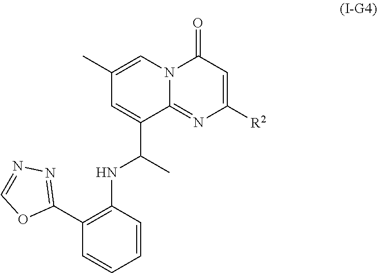

or a pharmaceutically acceptable salt thereof.

In some embodiments, the compound of Formula (I), or a pharmaceutically acceptable salt thereof, is a compound of Formula (I-G5):

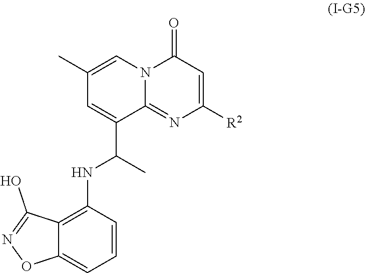

or a pharmaceutically acceptable salt thereof.

In some embodiments, the compound of Formula (I), or a pharmaceutically acceptable salt thereof, is a compound of Formula (I-G6):

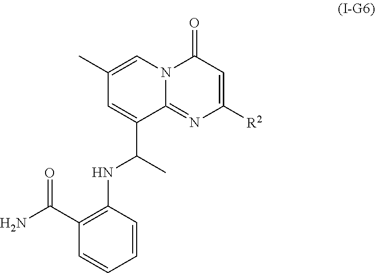

or a pharmaceutically acceptable salt thereof.

In some embodiments, the compound of Formula (I), or a pharmaceutically acceptable salt thereof, is a compound of Formula (I-H):

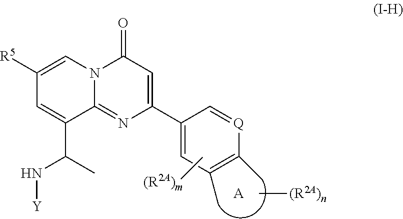

or a pharmaceutically acceptable salt thereof, wherein:
Ring A is a 5-6 membered heterocyclyl or 5-6 membered heteroaryl;
Q is N or CH;
m is 0, 1, 2, or 3; n is 0, 1, or 2; and m+n is 0, 1, 2, or 3.

In some embodiments, the compound of Formula (I), or a pharmaceutically acceptable salt thereof, is a compound of Formula (I-HH):

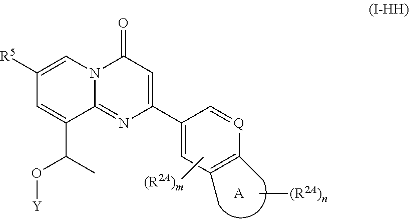

or a pharmaceutically acceptable salt thereof, wherein:
Ring A is a 5-6 membered heterocyclyl or 5-6 membered heteroaryl;
Q is N or CH;
m is 0, 1, 2, or 3; n is 0, 1, or 2; and m+n is 0, 1, 2, or 3.

In some embodiments, the compound of Formula (I), or a pharmaceutically acceptable salt thereof, is a compound of Formula (I-I):

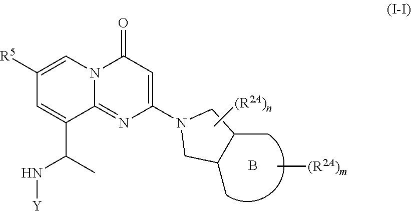

or a pharmaceutically acceptable salt thereof, wherein:
Ring B is a 5-6 membered heterocyclyl, 6 membered heteroaryl, or phenyl;
m is 0, 1, 2, or 3; n is 0, 1, or 2; and m+n is 0, 1, 2, or 3.

In some embodiments, the compound of Formula (I), or a pharmaceutically acceptable salt thereof, is a compound of Formula (I-II):

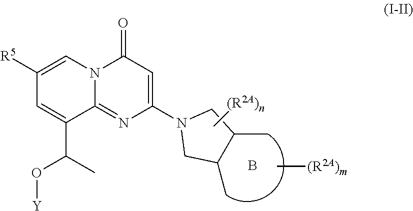

or a pharmaceutically acceptable salt thereof, wherein:
Ring B is a 5-6 membered heterocyclyl, 6 membered heteroaryl, or phenyl;
m is 0, 1, 2, or 3; n is 0, 1, or 2; and m+n is 0, 1, 2, or 3.

### Non-Limiting Exemplary Compounds

In some embodiments, the compound is selected from the group consisting of the Compounds 1-1413, or a pharmaceutically acceptable salt thereof.

In some embodiments, the compound is selected from the group consisting of the compounds delineated in Table A, or a pharmaceutically acceptable salt thereof.

TABLE A

| | Cmpd |
|---|---|
| Structure | No. |
| 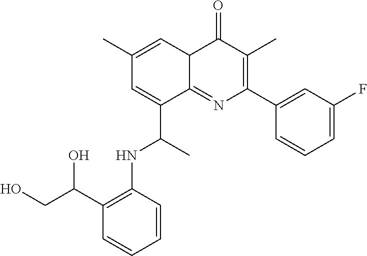 [SMILES: CC1=CC2C(=O)C(C)=C(c3cccc(F)c3)N=C2C(C(C)Nc2ccccc2C(O)CO)=C1] | 1 |
| 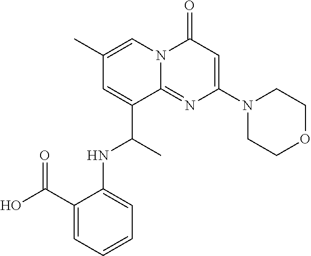 [SMILES: Cc1cc(C(C)Nc2ccccc2C(=O)O)c2nc(N3CCOCC3)cc(=O)n2c1] | 2 |
| 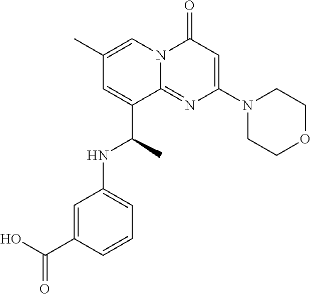 [SMILES: Cc1cc([C@@H](C)Nc2cccc(C(=O)O)c2)c2nc(N3CCOCC3)cc(=O)n2c1] | 3 |
| 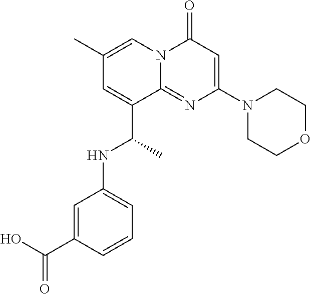 [SMILES: Cc1cc([C@H](C)Nc2cccc(C(=O)O)c2)c2nc(N3CCOCC3)cc(=O)n2c1] | 4 |
| 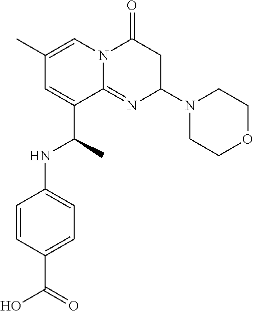 [SMILES: CC1=CN2C(=O)CC(N3CCOCC3)N=C2C([C@@H](C)Nc2ccc(C(=O)O)cc2)=C1] | 5 |
| 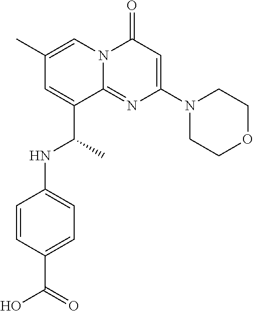 [SMILES: Cc1cc([C@H](C)Nc2ccc(C(=O)O)cc2)c2nc(N3CCOCC3)cc(=O)n2c1] | 6 |
| 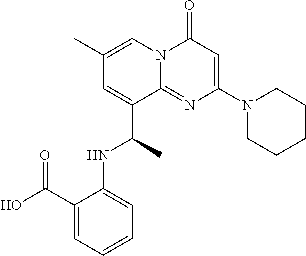 [SMILES: Cc1cc([C@@H](C)Nc2ccccc2C(=O)O)c2nc(N3CCCCC3)cc(=O)n2c1] | 7 |
| 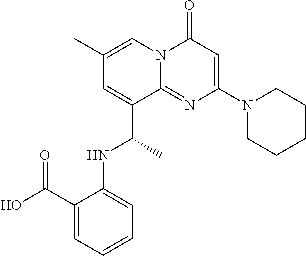 [SMILES: Cc1cc([C@H](C)Nc2ccccc2C(=O)O)c2nc(N3CCCCC3)cc(=O)n2c1] | 8 |
| 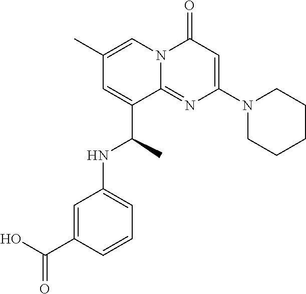 [SMILES: Cc1cc([C@@H](C)Nc2cccc(C(=O)O)c2)c2nc(N3CCCCC3)cc(=O)n2c1] | 9 |
| 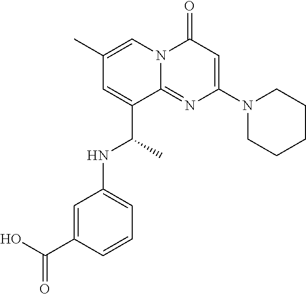 [SMILES: Cc1cc([C@H](C)Nc2cccc(C(=O)O)c2)c2nc(N3CCCCC3)cc(=O)n2c1] | 10 |
| 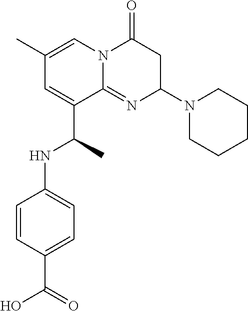 [SMILES: CC1=CN2C(=O)CC(N3CCCCC3)N=C2C([C@@H](C)Nc2ccc(C(=O)O)cc2)=C1] | 11 |
| 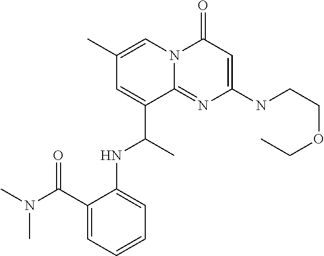 [SMILES: CCOCCNc1cc(=O)n2cc(C)cc(C(C)Nc3ccccc3C(=O)N(C)C)c2n1] | 12 |
| 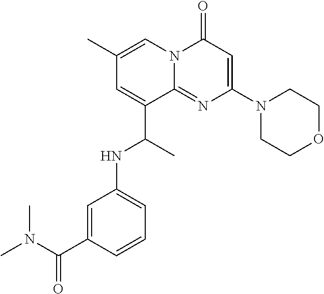 [SMILES: Cc1cc(C(C)Nc2cccc(C(=O)N(C)C)c2)c2nc(N3CCOCC3)cc(=O)n2c1] | 13 |
| 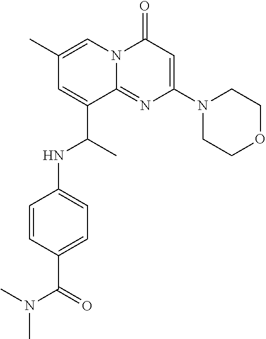 [SMILES: Cc1cc(C(C)Nc2ccc(C(=O)N(C)C)cc2)c2nc(N3CCOCC3)cc(=O)n2c1] | 14 |
| 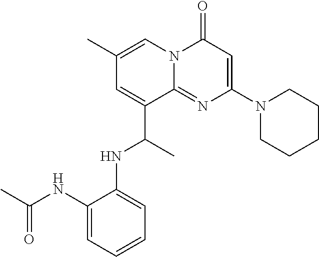 [SMILES: CC(=O)Nc1ccccc1NC(C)c1cc(C)cn2c(=O)cc(N3CCCCC3)nc12] | 15 |
| 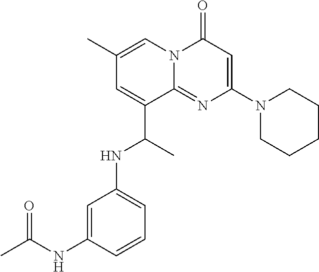 [SMILES: CC(=O)Nc1cccc(NC(C)c2cc(C)cn3c(=O)cc(N4CCCCC4)nc23)c1] | 16 |
| 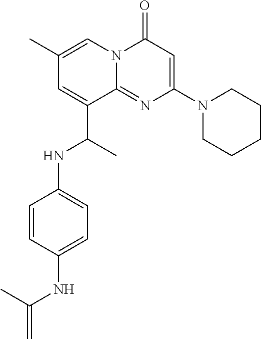 [SMILES: C=C(C)Nc1ccc(NC(C)c2cc(C)cn3c(=O)cc(N4CCCCC4)nc23)cc1] | 17 |
| 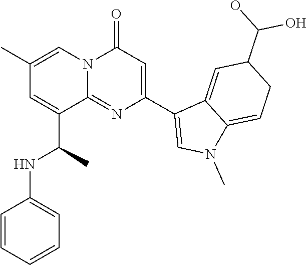 [SMILES: Cc1cc([C@@H](C)Nc2ccccc2)c2nc(-c3cn(C)c4c3=CC(C(O)O)CC=4)cc(=O)n2c1] | 18 |
| 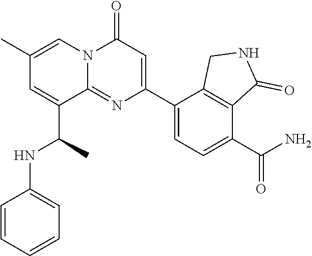 [SMILES: Cc1cc([C@@H](C)Nc2ccccc2)c2nc(-c3ccc(C(N)=O)c4c3CNC4=O)cc(=O)n2c1] | 19 |
| 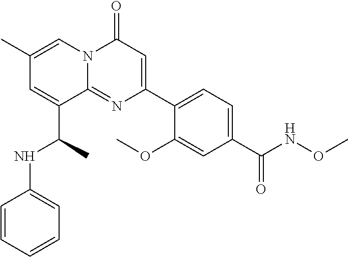 | 20 |
| 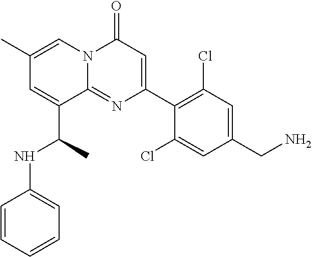 [SMILES: Cc1cc([C@@H](C)Nc2ccccc2)c2nc(-c3c(Cl)cc(CN)cc3Cl)cc(=O)n2c1] | 21 |
| 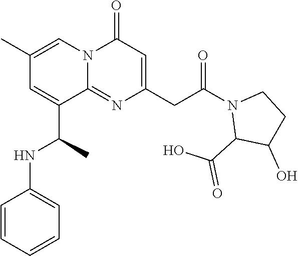 [SMILES: Cc1cc([C@@H](C)Nc2ccccc2)c2nc(CC(=O)N3CCC(O)C3C(=O)O)cc(=O)n2c1] | 22 |
| 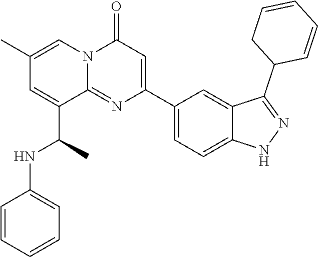 [SMILES: Cc1cc([C@@H](C)Nc2ccccc2)c2nc(-c3ccc4[nH]nc(C5C=CC=CC5)c4c3)cc(=O)n2c1] | 23 |
| 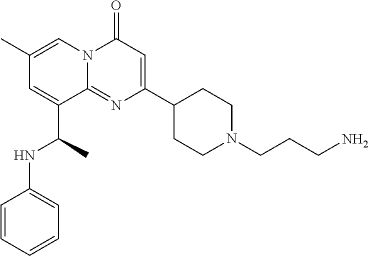 [SMILES: Cc1cc([C@@H](C)Nc2ccccc2)c2nc(C3CCN(CCCN)CC3)cc(=O)n2c1] | 24 |
| 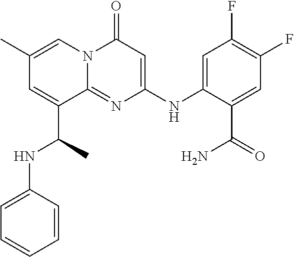 [SMILES: Cc1cc([C@@H](C)Nc2ccccc2)c2nc(Nc3cc(F)c(F)cc3C(N)=O)cc(=O)n2c1] | 25 |
| 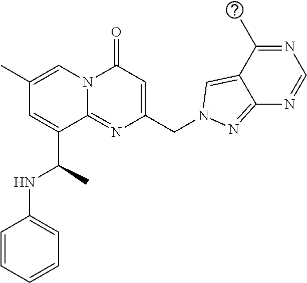 [SMILES: Cc1cc([C@@H](C)Nc2ccccc2)c2nc(Cn3cc4c(C)ncnc4n3)cc(=O)n2c1] | 26 |
| 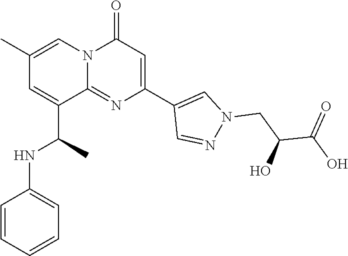 [SMILES: Cc1cc([C@@H](C)Nc2ccccc2)c2nc(-c3cnn(C[C@H](O)C(=O)O)c3)cc(=O)n2c1] | 27 |
| 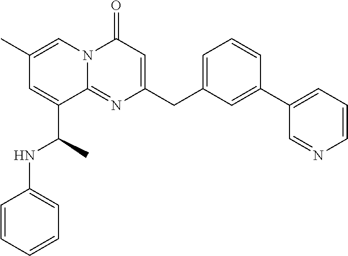 [SMILES: Cc1cc([C@@H](C)Nc2ccccc2)c2nc(Cc3cccc(-c4cccnc4)c3)cc(=O)n2c1] | 28 |
| 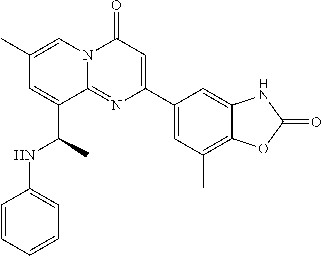 [SMILES: Cc1cc([C@@H](C)Nc2ccccc2)c2nc(-c3cc(C)c4oc(=O)[nH]c4c3)cc(=O)n2c1] | 29 |
| 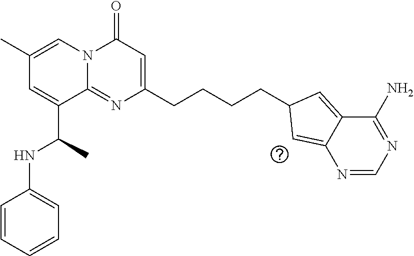 [SMILES: Cc1cc([C@@H](C)Nc2ccccc2)c2nc(CCCCC3C=c4ncnc(N)c4=C3)cc(=O)n2c1] | 30 |
| 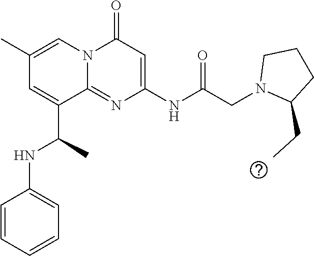 [SMILES: CC[C@@H]1CCCN1CC(=O)Nc1cc(=O)n2cc(C)cc([C@@H](C)Nc3ccccc3)c2n1] | 31 |
| 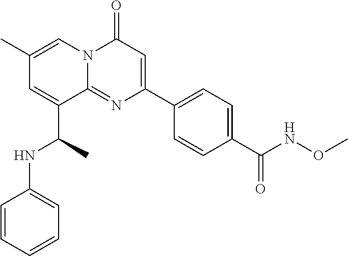 | 32 |
| 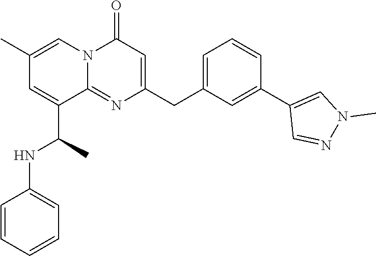 | 33 |
| 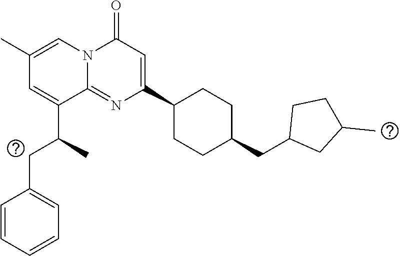 | 34 |
| 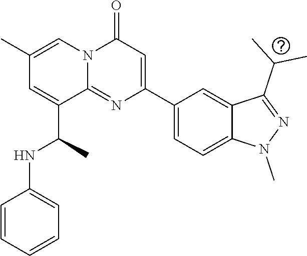 [SMILES: Cc1cc([C@@H](C)Nc2ccccc2)c2nc(-c3ccc4c(c3)c(C(C)C)nn4C)cc(=O)n2c1] | 35 |
| 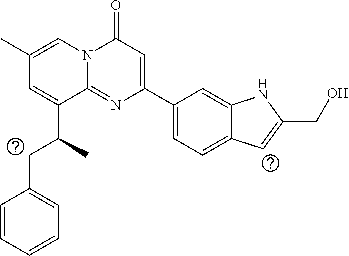 [SMILES: Cc1cc([C@@H](C)Cc2ccccc2)c2nc(-c3ccc4cc(CO)[nH]c4c3)cc(=O)n2c1] | 36 |
| 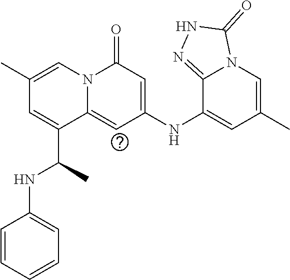 [SMILES: Cc1cc([C@@H](C)Nc2ccccc2)c2cc(Nc3cc(C)cn4c(=O)[nH]nc34)cc(=O)n2c1] | 37 |
| 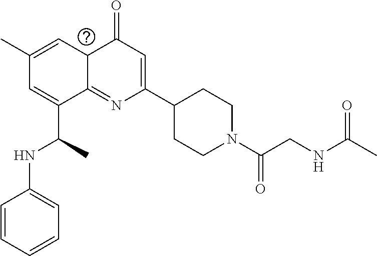 [SMILES: CC(=O)NCC(=O)N1CCC(C2=CC(=O)C3C=C(C)C=C([C@@H](C)Nc4ccccc4)C3=N2)CC1] | 38 |
| 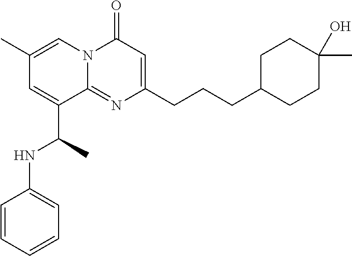 | 39 |
| 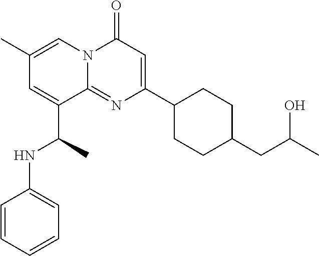 [SMILES: Cc1cc([C@@H](C)Nc2ccccc2)c2nc(C3CCC(CC(C)O)CC3)cc(=O)n2c1] | 40 |
| 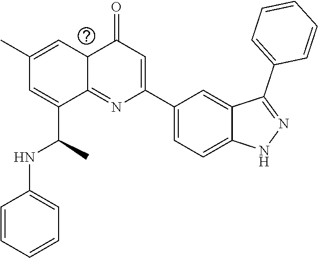 [SMILES: CC1=CC2C(=O)C=C(c3ccc4[nH]nc(-c5ccccc5)c4c3)N=C2C([C@@H](C)Nc2ccccc2)=C1] | 41 |
| 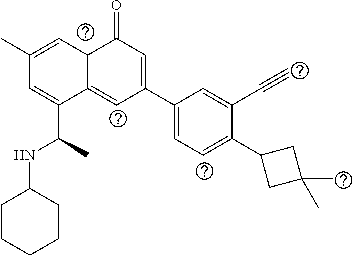 | 42 |
| 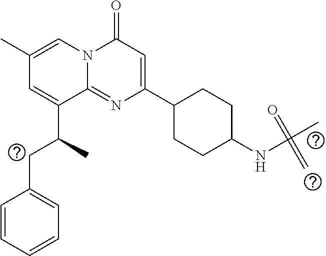 | 43 |
| 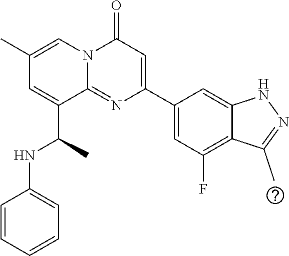 [SMILES: Cc1cc([C@@H](C)Nc2ccccc2)c2nc(-c3cc(F)c4c(C)n[nH]c4c3)cc(=O)n2c1] | 44 |
| 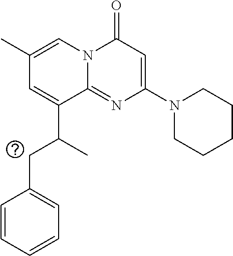 [SMILES: Cc1cc(C(C)Cc2ccccc2)c2nc(N3CCCCC3)cc(=O)n2c1] | 45 |
| 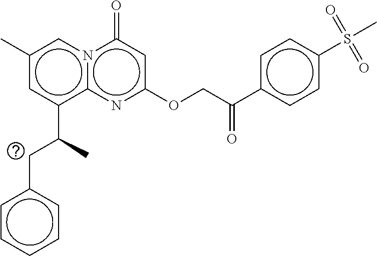 | 46 |
| 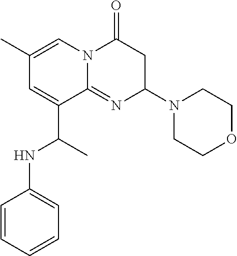 [SMILES: CC1=CN2C(=O)CC(N3CCOCC3)N=C2C(C(C)Nc2ccccc2)=C1] | 47 |
| 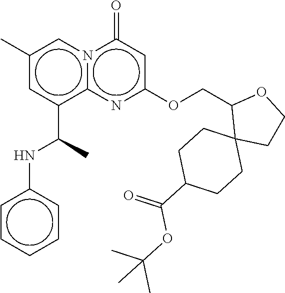 | 48 |
| 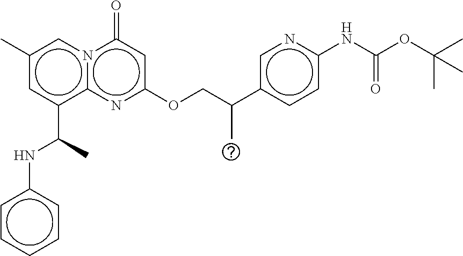 | 49 |
| 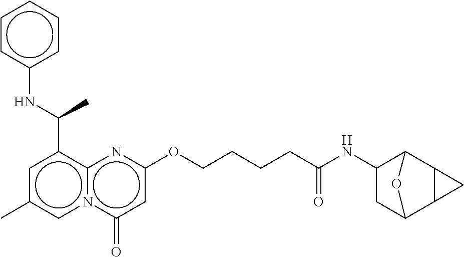 | 50 |
| 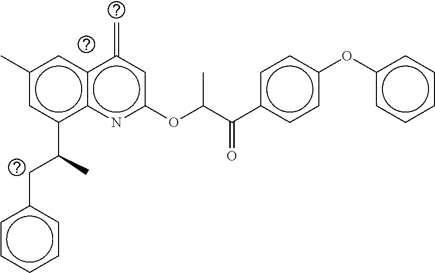 | 51 |
| 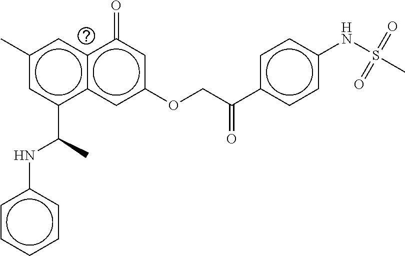 | 52 |
| 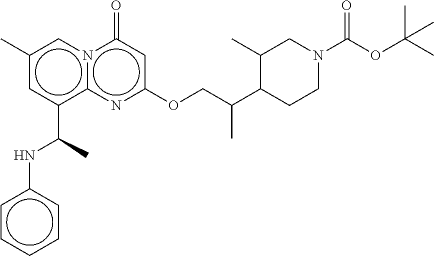 | 53 |
| 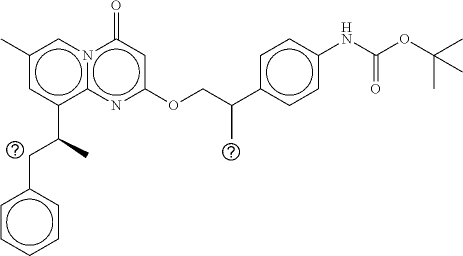 | 54 |
| 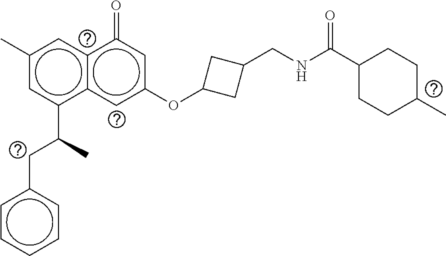 | 55 |
| 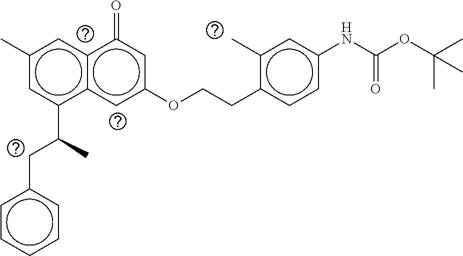 | 56 |
| 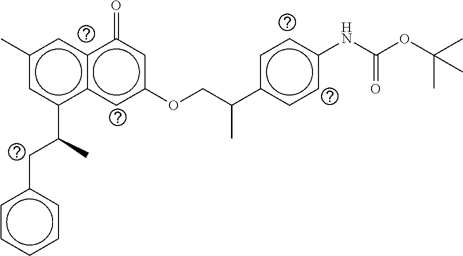 | 57 |
| 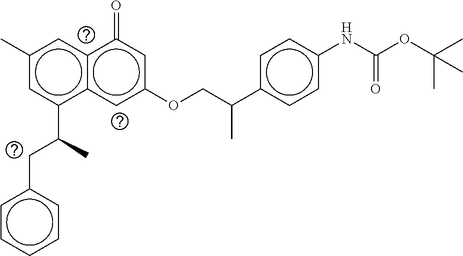 | 58 |
| 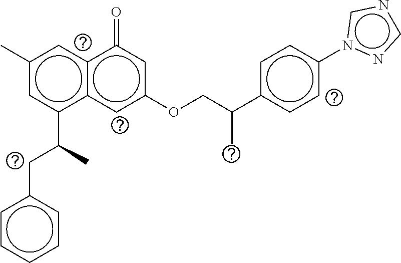 | 59 |
| 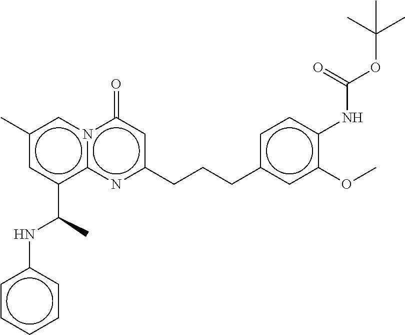 | 60 |
| 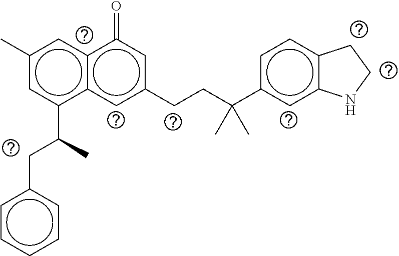 | 61 |
| 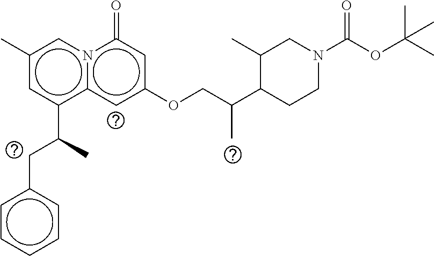 | 62 |
| 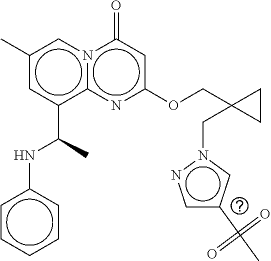 | 63 |
| 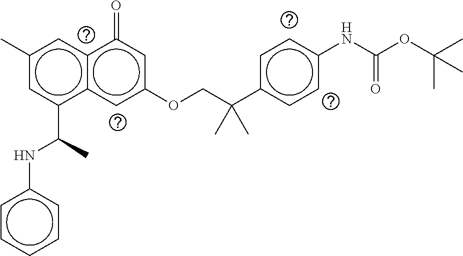 | 64 |
| 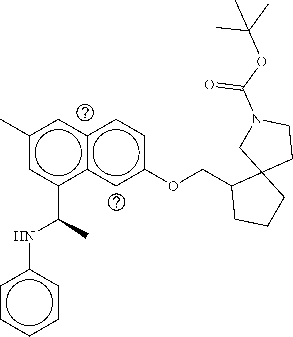 | 65 |
| 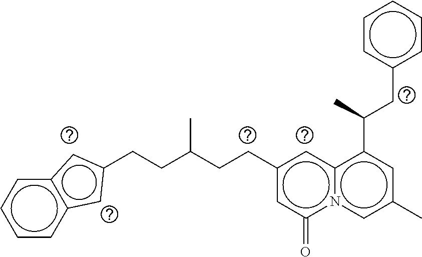 | 66 |
| 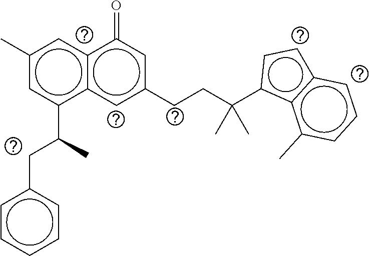 | 67 |
| 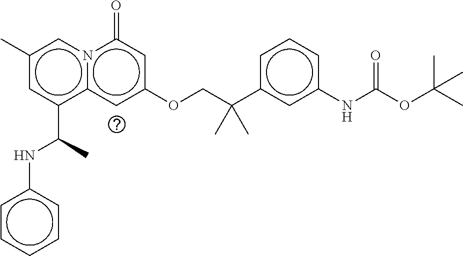 | 68 |
| 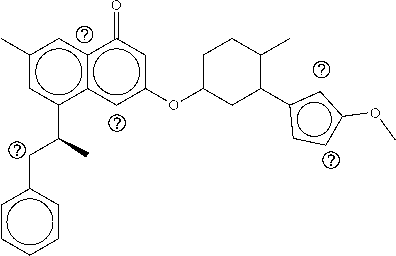 | 69 |
| 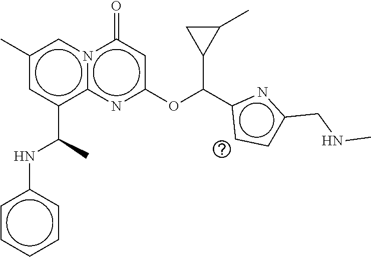 | 70 |
| 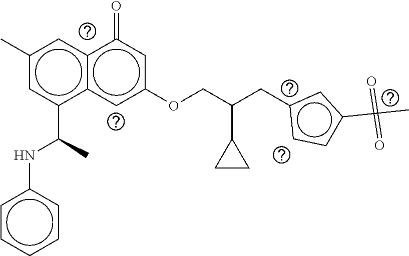 | 71 |
| 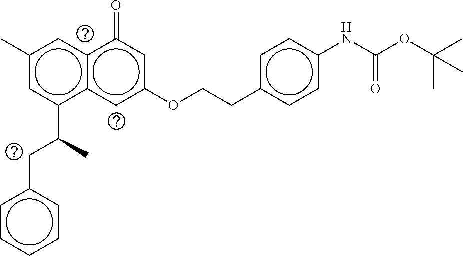 | 72 |
| 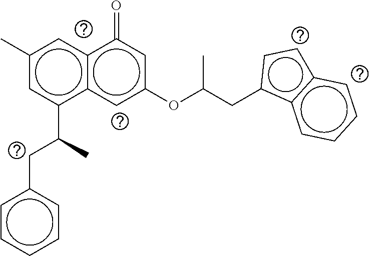 | 73 |
| 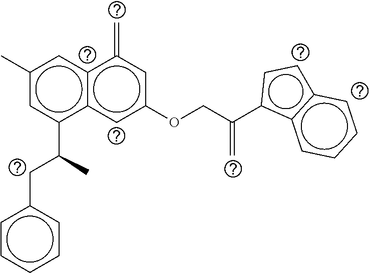 | 74 |
| 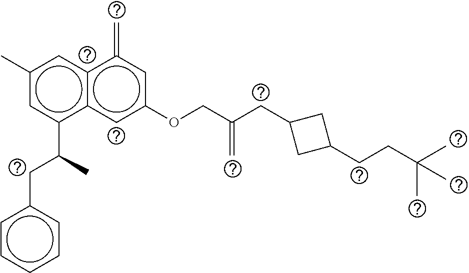 | 75 |
| 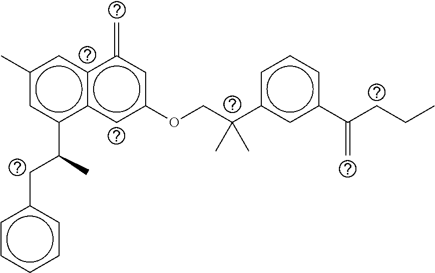 | 76 |
| 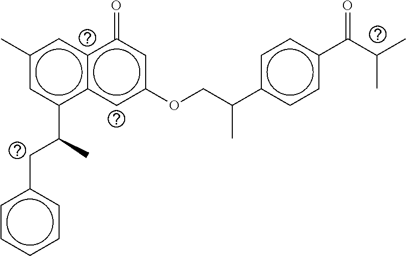 | 77 |
| 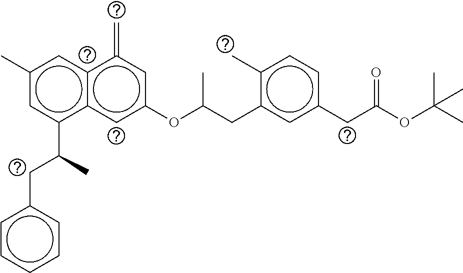 | 78 |
| 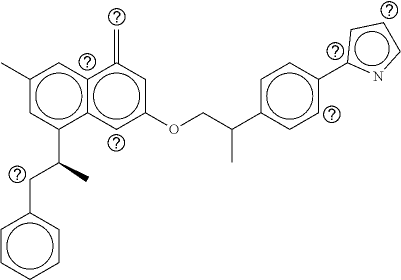 | 79 |
| 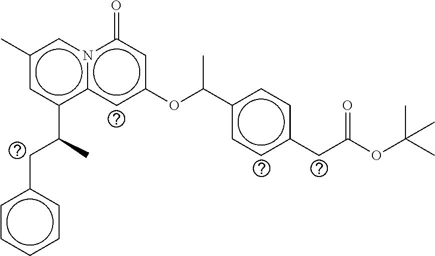 | 80 |
| 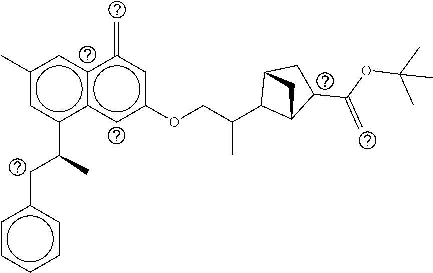 | 81 |
| 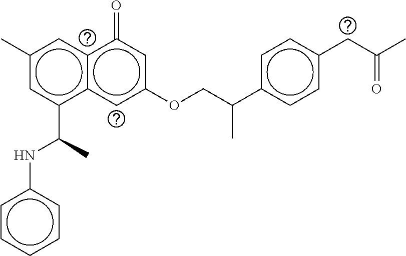 | 82 |
| 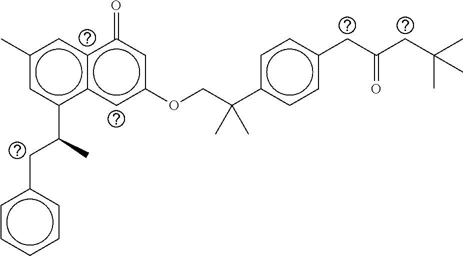 | 83 |
| 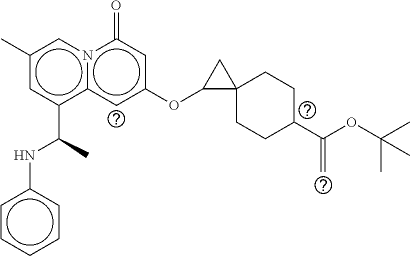 | 84 |
| 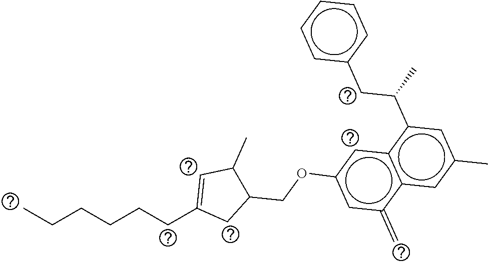 | 85 |
| 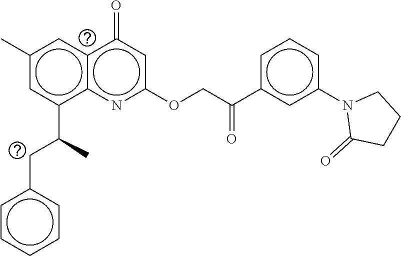 | 86 |
| 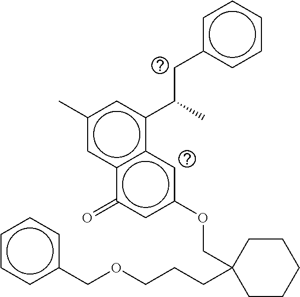 | 87 |
| 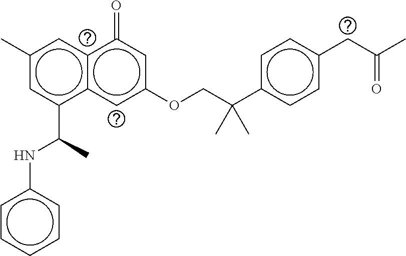 | 88 |
| 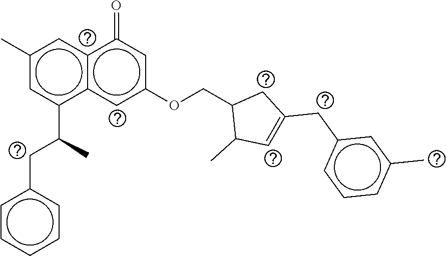 | 89 |
| 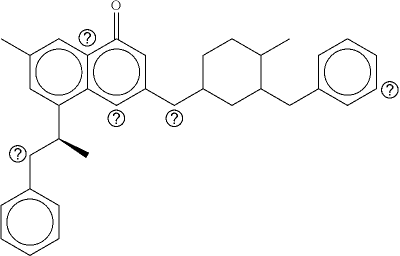 | 90 |
| 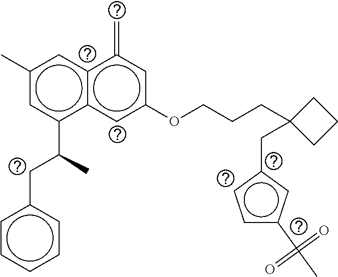 | 91 |
| 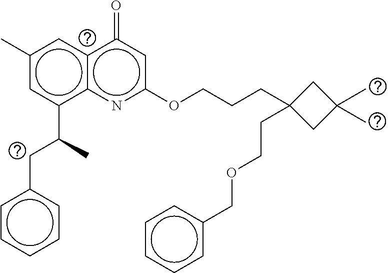 | 92 |
| 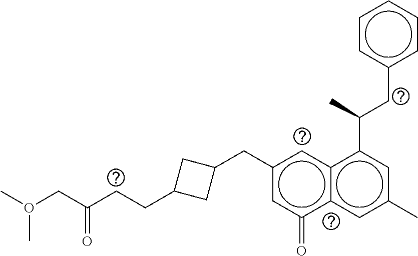 | 93 |
| 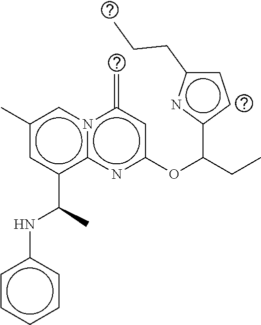 | 94 |
| 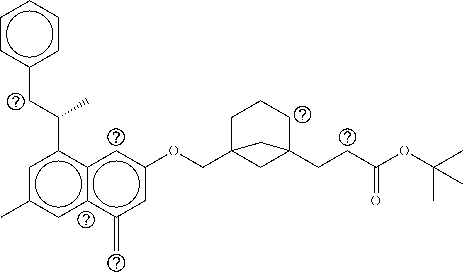 | 95 |
| 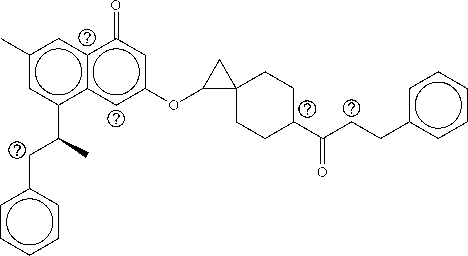 | 96 |
| 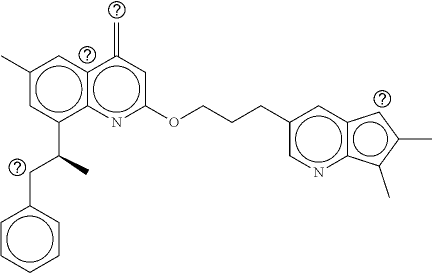 | 97 |
| 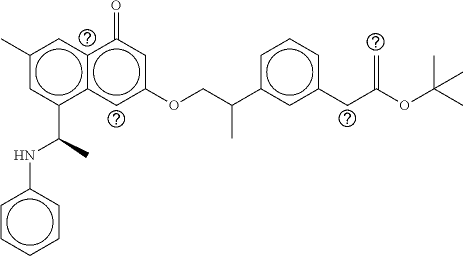 | 98 |
| 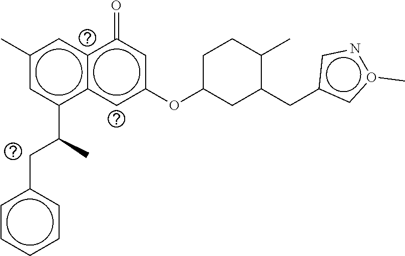 | 99 |
| 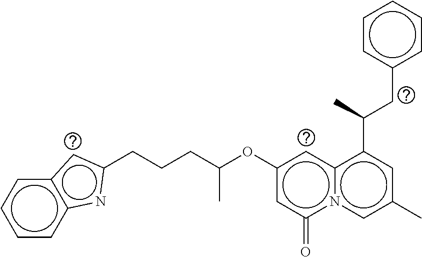 | 100 |
| 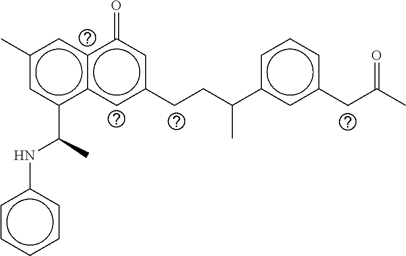 | 101 |
| 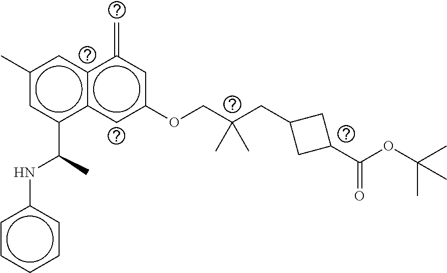 | 102 |
| 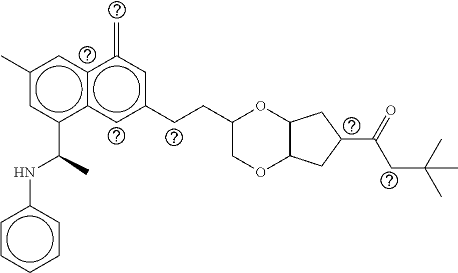 | 103 |
| 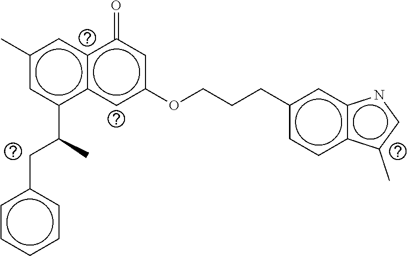 | 104 |
| 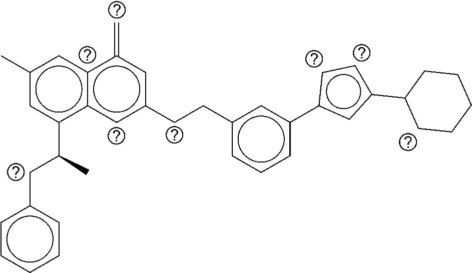 | 105 |
| 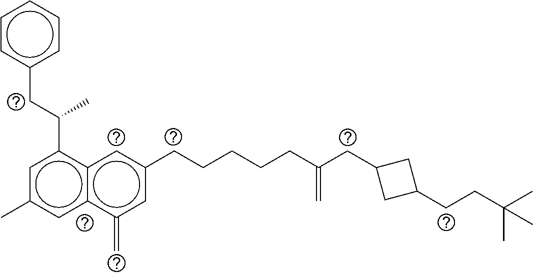 | 106 |
| 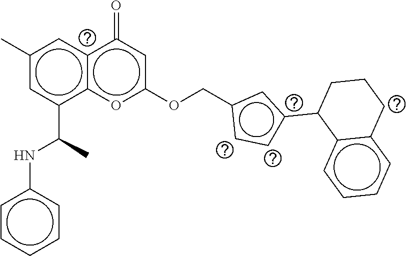 | 107 |
| 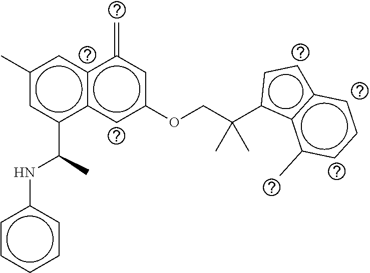 | 108 |
| 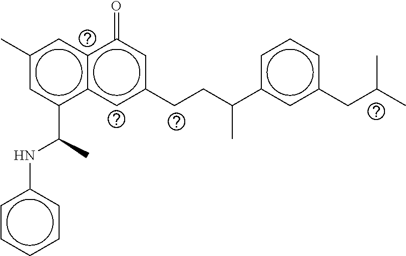 | 109 |
| 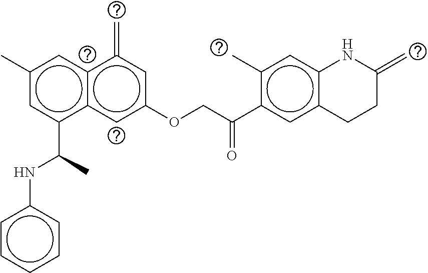 | 110 |
| 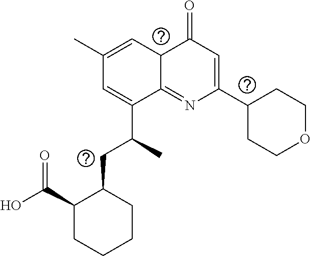 [SMILES: CC1=CC2C(=O)C=C(C3CCOCC3)N=C2C([C@@H](C)C[C@H]2CCCC[C@H]2C(=O)O)=C1] | 111 |
| 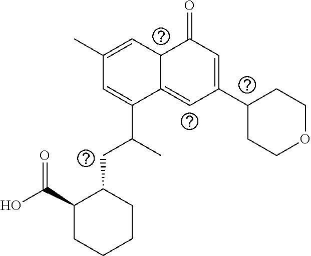 [SMILES: CC1=CC2C(=O)C=C(C3CCOCC3)C=C2C(C(C)C[C@@H]2CCCC[C@H]2C(=O)O)=C1] | 112 |
| 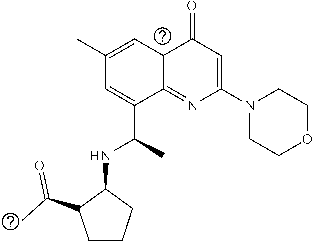 | 113 |
| 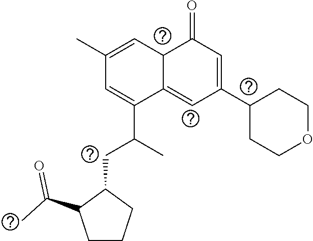 | 114 |
| 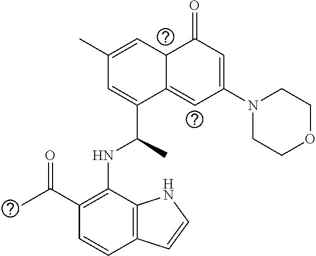 [SMILES: CC(=O)c1ccc2cc[nH]c2c1N[C@H](C)C1=CC(C)=CC2C(=O)C=C(N3CCOCC3)C=C12] | 115 |
| 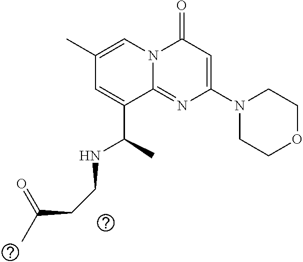 | 116 |
| 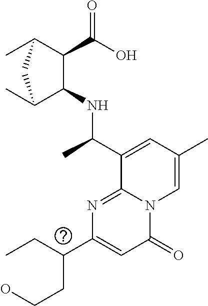 | 117 |
| 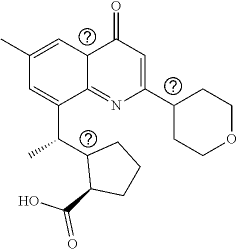 [SMILES: CC1=CC2C(=O)C=C(C3CCOCC3)N=C2C([C@@H](C)C2CCC[C@H]2C(=O)O)=C1] | 118 |
| 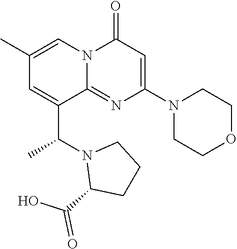 [SMILES: Cc1cc([C@@H](C)N2CCC[C@@H]2C(=O)O)c2nc(N3CCOCC3)cc(=O)n2c1] | 119 |
| 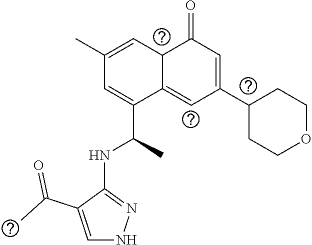 [SMILES: CC(=O)c1c[nH]nc1N[C@H](C)C1=CC(C)=CC2C(=O)C=C(C3CCOCC3)C=C12] | 120 |
| 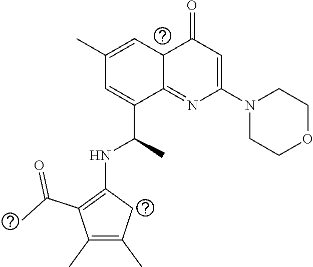 [SMILES: CC(=O)C1=C(N[C@H](C)C2=CC(C)=CC3C(=O)C=C(N4CCOCC4)N=C23)CC(C)=C1C] | 121 |
| 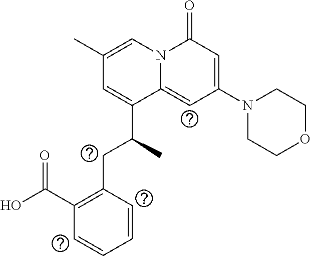 [SMILES: Cc1cc([C@@H](C)Cc2ccccc2C(=O)O)c2cc(N3CCOCC3)cc(=O)n2c1] | 122 |
| 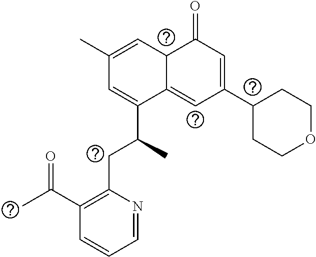 [SMILES: CC(=O)c1cccnc1C[C@H](C)C1=CC(C)=CC2C(=O)C=C(C3CCOCC3)C=C12] | 123 |
| 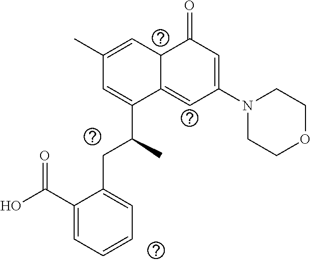 [SMILES: CC1=CC2C(=O)C=C(N3CCOCC3)C=C2C([C@@H](C)Cc2ccccc2C(=O)O)=C1] | 124 |
| 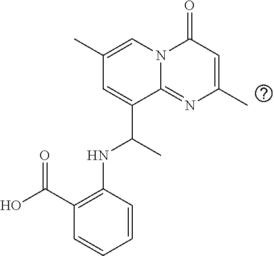 [SMILES: Cc1cc(C(C)Nc2ccccc2C(=O)O)c2nc(C)cc(=O)n2c1] | 125 |
| 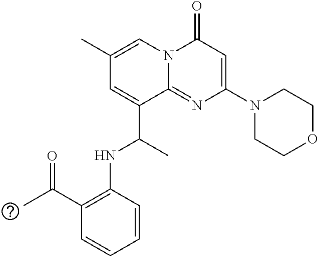 [SMILES: CC(=O)c1ccccc1NC(C)c1cc(C)cn2c(=O)cc(N3CCOCC3)nc12] | 126 |
| 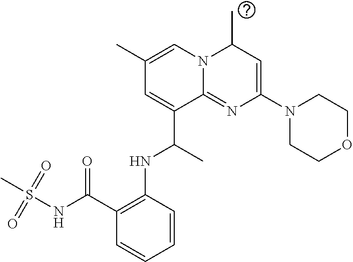 [SMILES: CC1=CN2C(=NC(N3CCOCC3)=CC2C)C(C(C)Nc2ccccc2C(=O)NS(C)(=O)=O)=C1] | 127 |
| 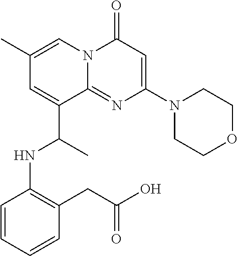 [SMILES: Cc1cc(C(C)Nc2ccccc2CC(=O)O)c2nc(N3CCOCC3)cc(=O)n2c1] | 128 |
| 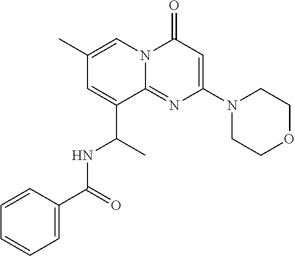 [SMILES: Cc1cc(C(C)NC(=O)c2ccccc2)c2nc(N3CCOCC3)cc(=O)n2c1] | 129 |
| 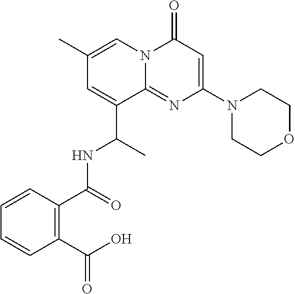 [SMILES: Cc1cc(C(C)NC(=O)c2ccccc2C(=O)O)c2nc(N3CCOCC3)cc(=O)n2c1] | 130 |
| 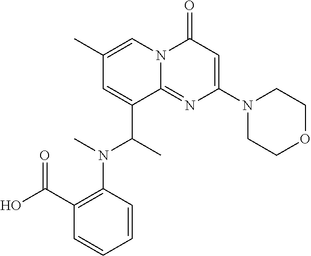 [SMILES: Cc1cc(C(C)N(C)c2ccccc2C(=O)O)c2nc(N3CCOCC3)cc(=O)n2c1] | 131 |
| 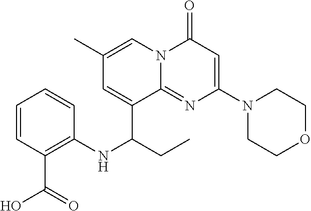 [SMILES: CCC(Nc1ccccc1C(=O)O)c1cc(C)cn2c(=O)cc(N3CCOCC3)nc12] | 132 |
| 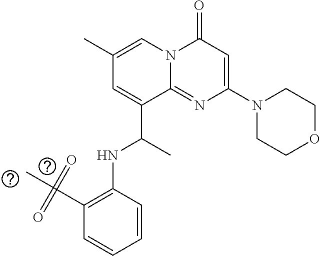 | 133 |
| 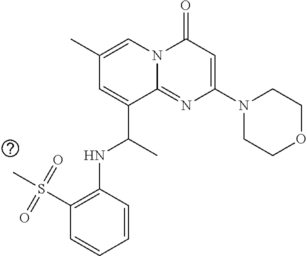 [SMILES: Cc1cc(C(C)Nc2ccccc2S(C)(=O)=O)c2nc(N3CCOCC3)cc(=O)n2c1] | 134 |
| 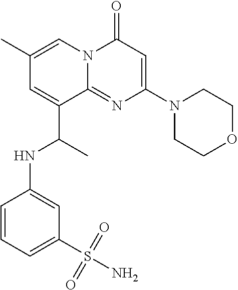 [SMILES: Cc1cc(C(C)Nc2cccc(S(N)(=O)=O)c2)c2nc(N3CCOCC3)cc(=O)n2c1] | 135 |
| 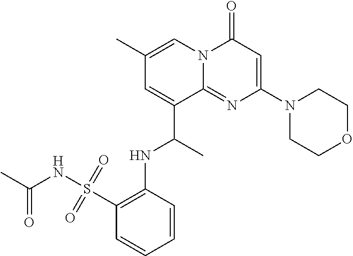 [SMILES: CC(=O)NS(=O)(=O)c1ccccc1NC(C)c1cc(C)cn2c(=O)cc(N3CCOCC3)nc12] | 136 |
| 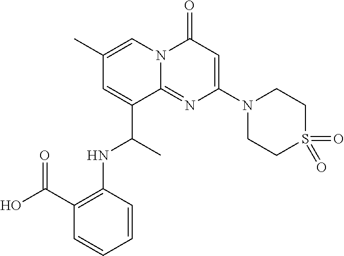 [SMILES: Cc1cc(C(C)Nc2ccccc2C(=O)O)c2nc(N3CCS(=O)(=O)CC3)cc(=O)n2c1] | 137 |
| 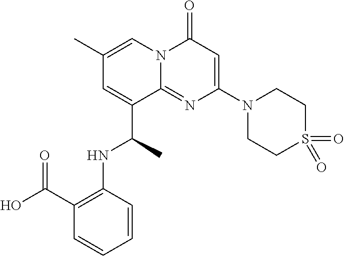 [SMILES: Cc1cc([C@@H](C)Nc2ccccc2C(=O)O)c2nc(N3CCS(=O)(=O)CC3)cc(=O)n2c1] | 138 |
| 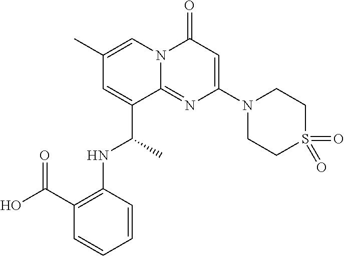 [SMILES: Cc1cc([C@H](C)Nc2ccccc2C(=O)O)c2nc(N3CCS(=O)(=O)CC3)cc(=O)n2c1] | 139 |
| 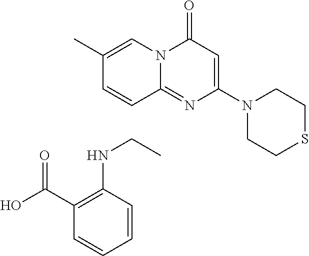 | 140 |
| 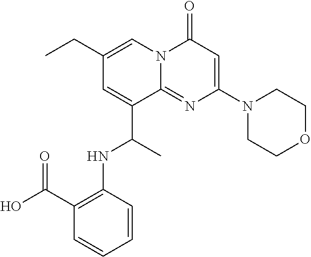 [SMILES: CCc1cc(C(C)Nc2ccccc2C(=O)O)c2nc(N3CCOCC3)cc(=O)n2c1] | 141 |
| 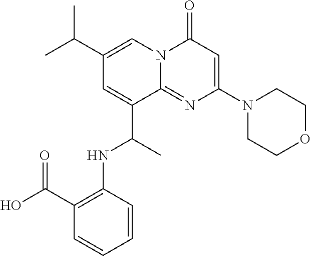 [SMILES: CC(C)c1cc(C(C)Nc2ccccc2C(=O)O)c2nc(N3CCOCC3)cc(=O)n2c1] | 142 |
| 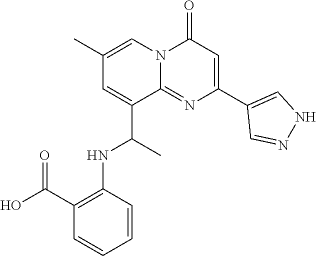 [SMILES: Cc1cc(C(C)Nc2ccccc2C(=O)O)c2nc(-c3cn[nH]c3)cc(=O)n2c1] | 143 |
| 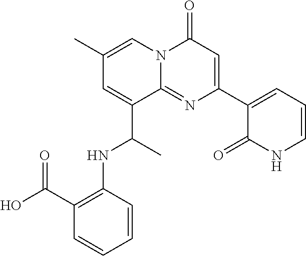 [SMILES: Cc1cc(C(C)Nc2ccccc2C(=O)O)c2nc(-c3ccc[nH]c3=O)cc(=O)n2c1] | 144 |
| 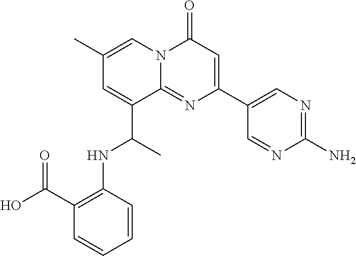 [SMILES: Cc1cc(C(C)Nc2ccccc2C(=O)O)c2nc(-c3cnc(N)nc3)cc(=O)n2c1] | 145 |
| 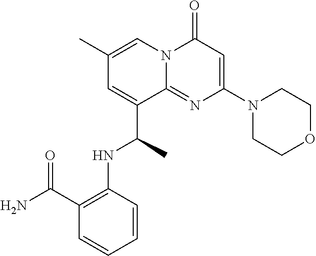 [SMILES: Cc1cc([C@@H](C)Nc2ccccc2C(N)=O)c2nc(N3CCOCC3)cc(=O)n2c1] | 146 |
| 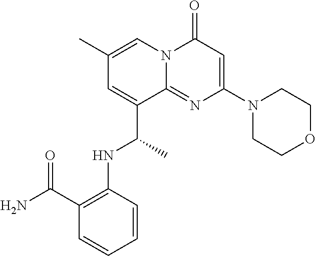 [SMILES: Cc1cc([C@H](C)Nc2ccccc2C(N)=O)c2nc(N3CCOCC3)cc(=O)n2c1] | 147 |
| 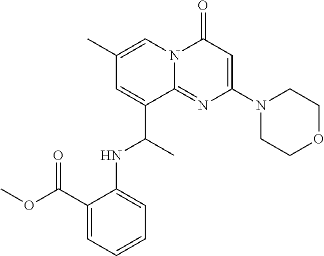 [SMILES: COC(=O)c1ccccc1NC(C)c1cc(C)cn2c(=O)cc(N3CCOCC3)nc12] | 148 |
| 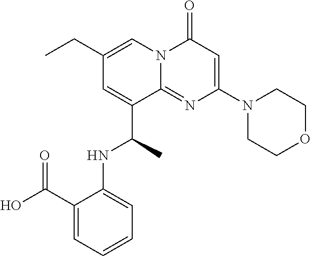 [SMILES: CCc1cc([C@@H](C)Nc2ccccc2C(=O)O)c2nc(N3CCOCC3)cc(=O)n2c1] | 149 |
| 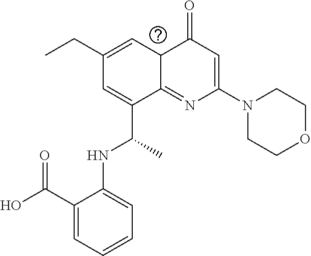 [SMILES: CCC1=CC2C(=O)C=C(N3CCOCC3)N=C2C([C@H](C)Nc2ccccc2C(=O)O)=C1] | 150 |
| 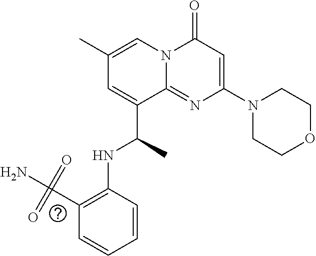 | 151 |
| 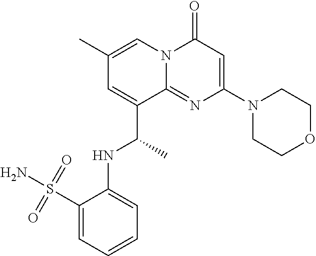 [SMILES: Cc1cc([C@H](C)Nc2ccccc2S(N)(=O)=O)c2nc(N3CCOCC3)cc(=O)n2c1] | 152 |
| 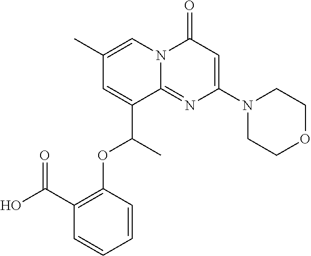 [SMILES: Cc1cc(C(C)Oc2ccccc2C(=O)O)c2nc(N3CCOCC3)cc(=O)n2c1] | 153 |
| 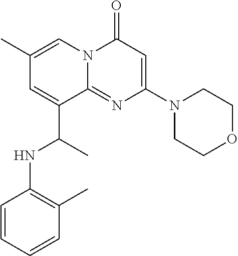 [SMILES: Cc1cc(C(C)Nc2ccccc2C)c2nc(N3CCOCC3)cc(=O)n2c1] | 154 |
| 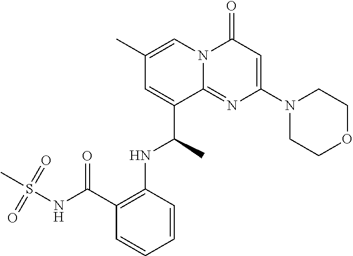 [SMILES: Cc1cc([C@@H](C)Nc2ccccc2C(=O)NS(C)(=O)=O)c2nc(N3CCOCC3)cc(=O)n2c1] | 155 |
| 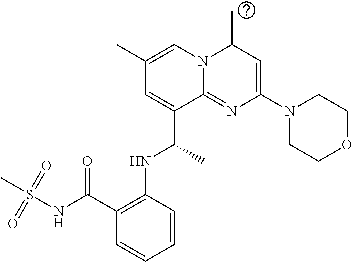 [SMILES: CC1=CN2C(=NC(N3CCOCC3)=CC2C)C([C@H](C)Nc2ccccc2C(=O)NS(C)(=O)=O)=C1] | 156 |
| 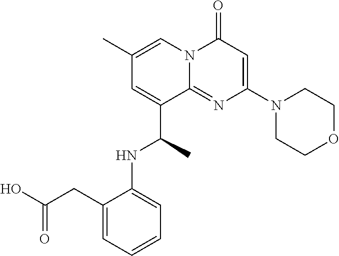 [SMILES: Cc1cc([C@@H](C)Nc2ccccc2CC(=O)O)c2nc(N3CCOCC3)cc(=O)n2c1] | 157 |
| 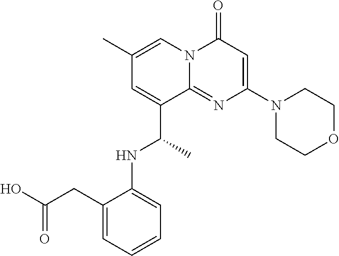 [SMILES: Cc1cc([C@H](C)Nc2ccccc2CC(=O)O)c2nc(N3CCOCC3)cc(=O)n2c1] | 158 |
| 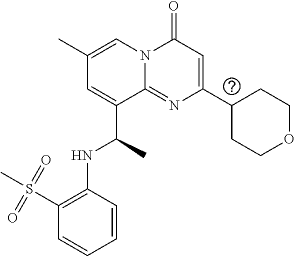 [SMILES: Cc1cc([C@@H](C)Nc2ccccc2S(C)(=O)=O)c2nc(C3CCOCC3)cc(=O)n2c1] | 159 |
| 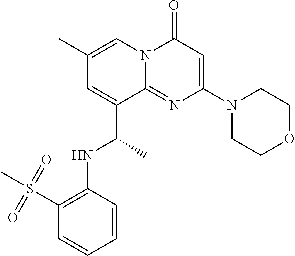 [SMILES: Cc1cc([C@H](C)Nc2ccccc2S(C)(=O)=O)c2nc(N3CCOCC3)cc(=O)n2c1] | 160 |
| 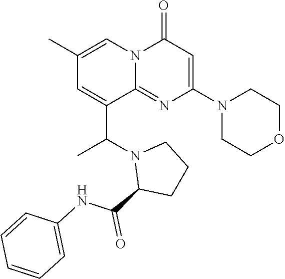 [SMILES: Cc1cc(C(C)N2CCC[C@H]2C(=O)Nc2ccccc2)c2nc(N3CCOCC3)cc(=O)n2c1] | 161 |
| 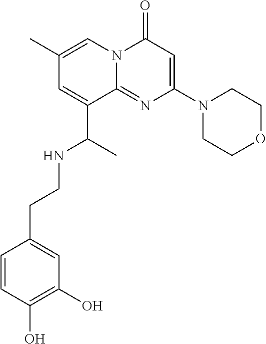 [SMILES: Cc1cc(C(C)NCCc2ccc(O)c(O)c2)c2nc(N3CCOCC3)cc(=O)n2c1] | 162 |
| 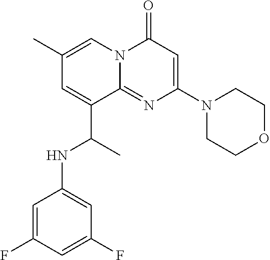 [SMILES: Cc1cc(C(C)Nc2cc(F)cc(F)c2)c2nc(N3CCOCC3)cc(=O)n2c1] | 163 |
| 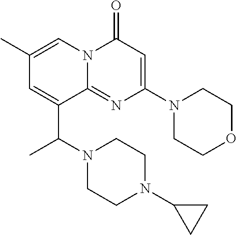 [SMILES: Cc1cc(C(C)N2CCN(C3CC3)CC2)c2nc(N3CCOCC3)cc(=O)n2c1] | 164 |
| 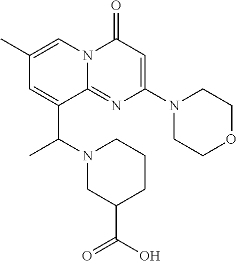 [SMILES: Cc1cc(C(C)N2CCCC(C(=O)O)C2)c2nc(N3CCOCC3)cc(=O)n2c1] | 165 |
| 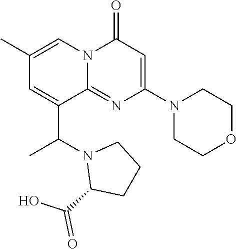 [SMILES: Cc1cc(C(C)N2CCC[C@@H]2C(=O)O)c2nc(N3CCOCC3)cc(=O)n2c1] | 166 |
| 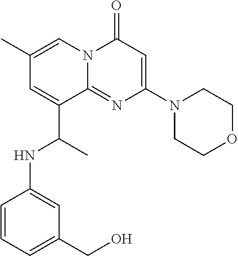 [SMILES: Cc1cc(C(C)Nc2cccc(CO)c2)c2nc(N3CCOCC3)cc(=O)n2c1] | 167 |
| 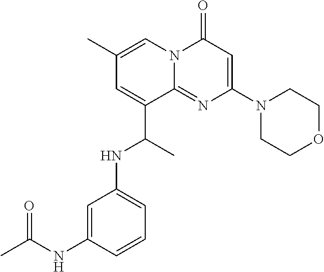 [SMILES: CC(=O)Nc1cccc(NC(C)c2cc(C)cn3c(=O)cc(N4CCOCC4)nc23)c1] | 168 |
| 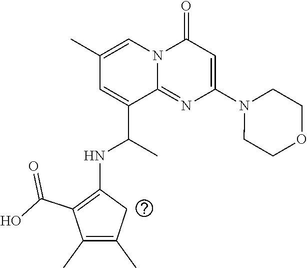 [SMILES: CC1=C(C)C(C(=O)O)=C(NC(C)c2cc(C)cn3c(=O)cc(N4CCOCC4)nc23)C1] | 169 |
| 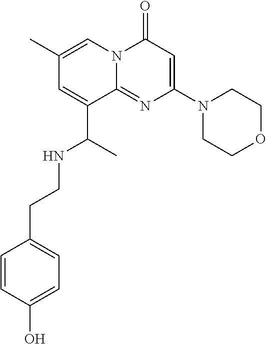 [SMILES: Cc1cc(C(C)NCCc2ccc(O)cc2)c2nc(N3CCOCC3)cc(=O)n2c1] | 170 |
| 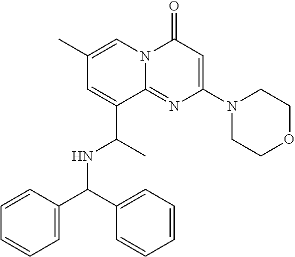 [SMILES: Cc1cc(C(C)NC(c2ccccc2)c2ccccc2)c2nc(N3CCOCC3)cc(=O)n2c1] | 171 |
| 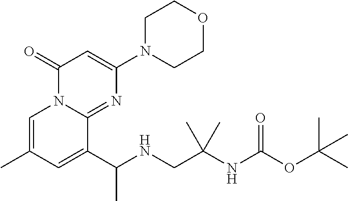 [SMILES: Cc1cc(C(C)NCC(C)(C)NC(=O)OC(C)(C)C)c2nc(N3CCOCC3)cc(=O)n2c1] | 172 |
| 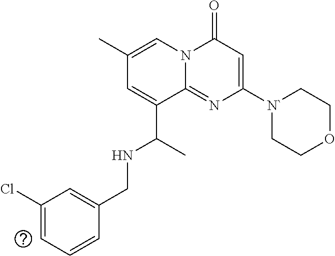 | 173 |
| 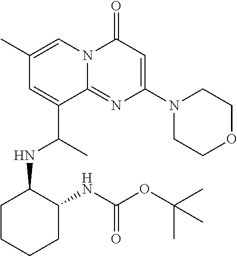 [SMILES: Cc1cc(C(C)N[C@@H]2CCCC[C@H]2NC(=O)OC(C)(C)C)c2nc(N3CCOCC3)cc(=O)n2c1] | 174 |
| 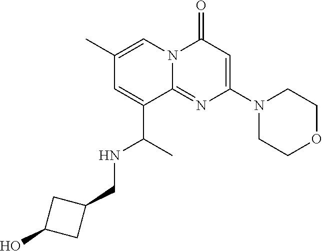 [SMILES: Cc1cc(C(C)NC[C@H]2C[C@@H](O)C2)c2nc(N3CCOCC3)cc(=O)n2c1] | 175 |
| 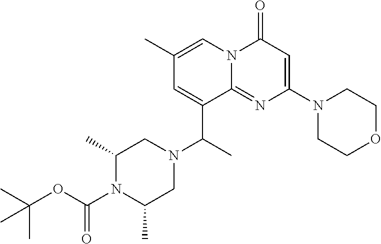 [SMILES: Cc1cc(C(C)N2C[C@@H](C)N(C(=O)OC(C)(C)C)[C@@H](C)C2)c2nc(N3CCOCC3)cc(=O)n2c1] | 176 |
| 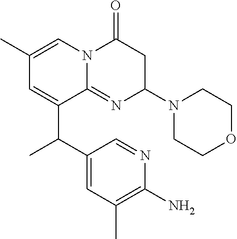 [SMILES: CC1=CN2C(=O)CC(N3CCOCC3)N=C2C(C(C)c2cnc(N)c(C)c2)=C1] | 177 |
| 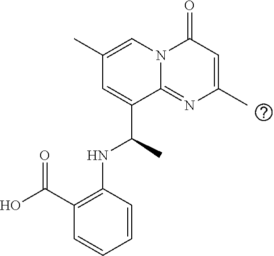 [SMILES: Cc1cc([C@@H](C)Nc2ccccc2C(=O)O)c2nc(C)cc(=O)n2c1] | 178 |
| 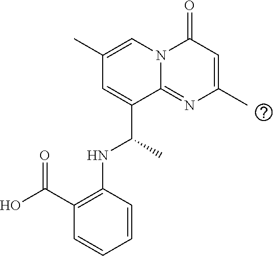 [SMILES: Cc1cc([C@H](C)Nc2ccccc2C(=O)O)c2nc(C)cc(=O)n2c1] | 179 |
| 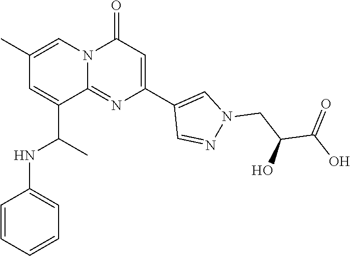 [SMILES: Cc1cc(C(C)Nc2ccccc2)c2nc(-c3cnn(C[C@H](O)C(=O)O)c3)cc(=O)n2c1] | 180 |
| 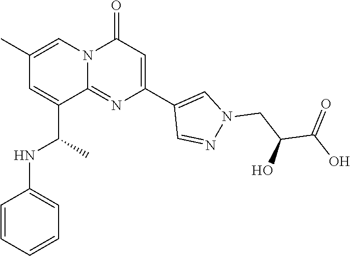 [SMILES: Cc1cc([C@H](C)Nc2ccccc2)c2nc(-c3cnn(C[C@H](O)C(=O)O)c3)cc(=O)n2c1] | 181 |
| 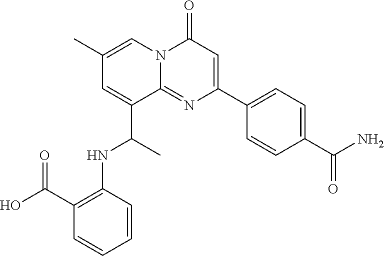 [SMILES: Cc1cc(C(C)Nc2ccccc2C(=O)O)c2nc(-c3ccc(C(N)=O)cc3)cc(=O)n2c1] | 182 |
| 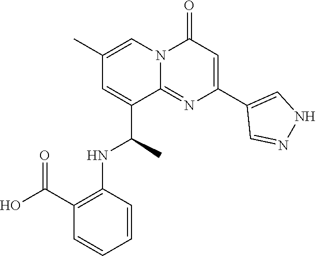 [SMILES: Cc1cc([C@@H](C)Nc2ccccc2C(=O)O)c2nc(-c3cn[nH]c3)cc(=O)n2c1] | 183 |
| 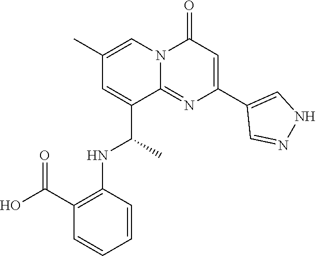 [SMILES: Cc1cc([C@H](C)Nc2ccccc2C(=O)O)c2nc(-c3cn[nH]c3)cc(=O)n2c1] | 184 |
| 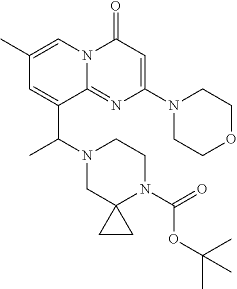 [SMILES: Cc1cc(C(C)N2CCN(C(=O)OC(C)(C)C)C3(CC3)C2)c2nc(N3CCOCC3)cc(=O)n2c1] | 185 |
| 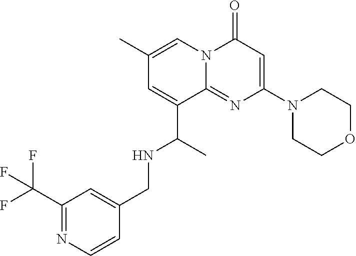 [SMILES: Cc1cc(C(C)NCc2ccnc(C(F)(F)F)c2)c2nc(N3CCOCC3)cc(=O)n2c1] | 186 |
| 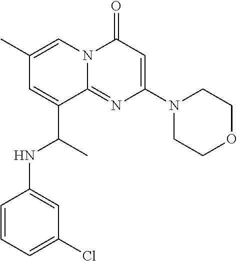 [SMILES: Cc1cc(C(C)Nc2cccc(Cl)c2)c2nc(N3CCOCC3)cc(=O)n2c1] | 187 |
| 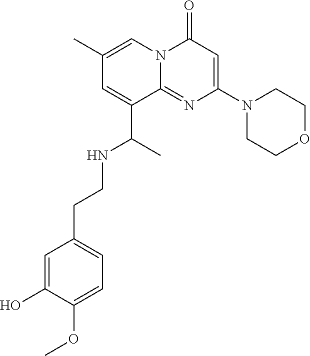 [SMILES: COc1ccc(CCNC(C)c2cc(C)cn3c(=O)cc(N4CCOCC4)nc23)cc1O] | 188 |
| 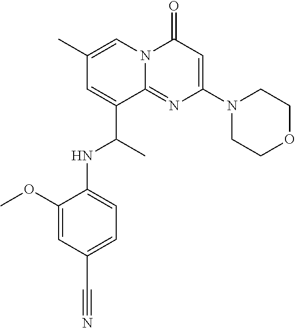 [SMILES: COc1cc(C#N)ccc1NC(C)c1cc(C)cn2c(=O)cc(N3CCOCC3)nc12] | 189 |
| 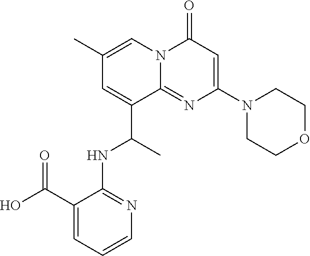 [SMILES: Cc1cc(C(C)Nc2ncccc2C(=O)O)c2nc(N3CCOCC3)cc(=O)n2c1] | 190 |
| 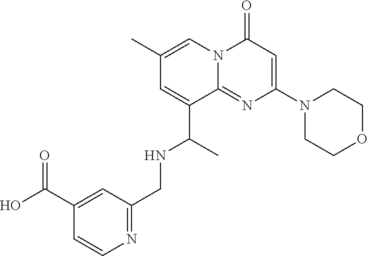 [SMILES: Cc1cc(C(C)NCc2cc(C(=O)O)ccn2)c2nc(N3CCOCC3)cc(=O)n2c1] | 191 |
| 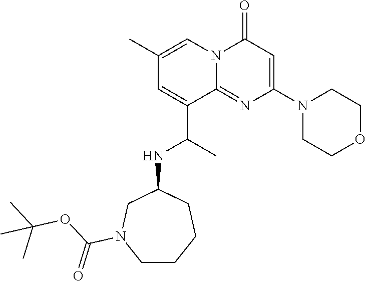 [SMILES: Cc1cc(C(C)N[C@H]2CCCCN(C(=O)OC(C)(C)C)C2)c2nc(N3CCOCC3)cc(=O)n2c1] | 192 |
| 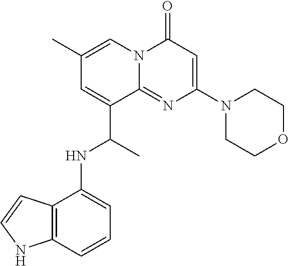 [SMILES: Cc1cc(C(C)Nc2cccc3[nH]ccc23)c2nc(N3CCOCC3)cc(=O)n2c1] | 193 |
| 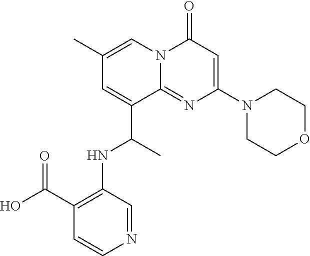 [SMILES: Cc1cc(C(C)Nc2cnccc2C(=O)O)c2nc(N3CCOCC3)cc(=O)n2c1] | 194 |
| 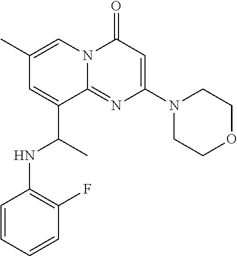 [SMILES: Cc1cc(C(C)Nc2ccccc2F)c2nc(N3CCOCC3)cc(=O)n2c1] | 195 |
| 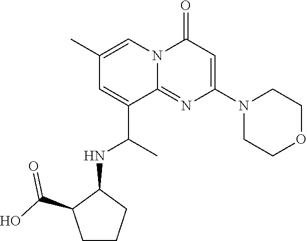 [SMILES: Cc1cc(C(C)N[C@H]2CCC[C@H]2C(=O)O)c2nc(N3CCOCC3)cc(=O)n2c1] | 196 |
| 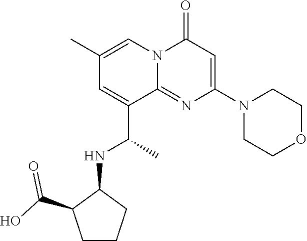 [SMILES: Cc1cc([C@H](C)N[C@H]2CCC[C@H]2C(=O)O)c2nc(N3CCOCC3)cc(=O)n2c1] | 197 |
| 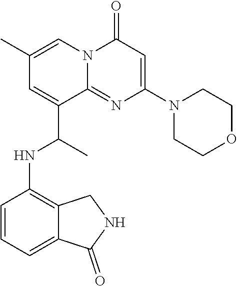 [SMILES: Cc1cc(C(C)Nc2cccc3c2CNC3=O)c2nc(N3CCOCC3)cc(=O)n2c1] | 198 |
| 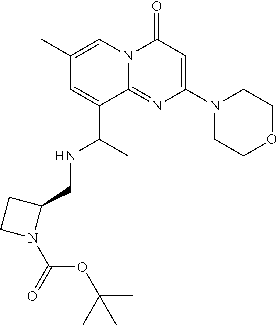 [SMILES: Cc1cc(C(C)NC[C@@H]2CCN2C(=O)OC(C)(C)C)c2nc(N3CCOCC3)cc(=O)n2c1] | 199 |
| 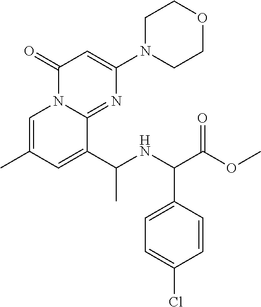 | 200 |
| 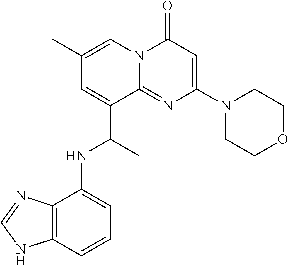 [SMILES: Cc1cc(C(C)Nc2cccc3[nH]cnc23)c2nc(N3CCOCC3)cc(=O)n2c1] | 201 |
| 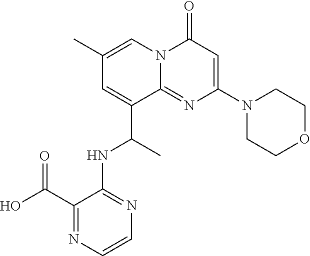 [SMILES: Cc1cc(C(C)Nc2nccnc2C(=O)O)c2nc(N3CCOCC3)cc(=O)n2c1] | 202 |
| 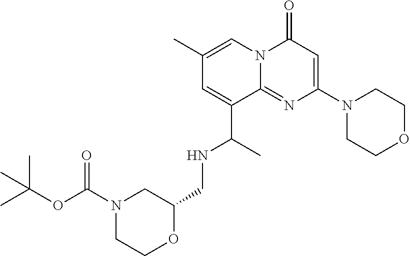 [SMILES: Cc1cc(C(C)NC[C@H]2CN(C(=O)OC(C)(C)C)CCO2)c2nc(N3CCOCC3)cc(=O)n2c1] | 203 |
| 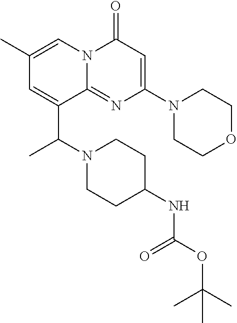 [SMILES: Cc1cc(C(C)N2CCC(NC(=O)OC(C)(C)C)CC2)c2nc(N3CCOCC3)cc(=O)n2c1] | 204 |
| 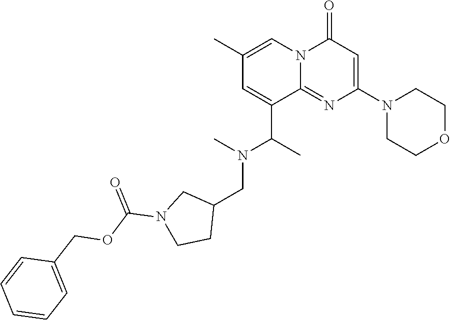 [SMILES: Cc1cc(C(C)N(C)CC2CCN(C(=O)OCc3ccccc3)C2)c2nc(N3CCOCC3)cc(=O)n2c1] | 205 |
| 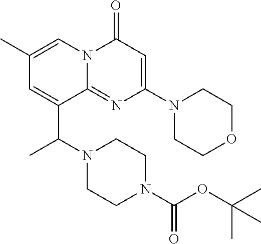 [SMILES: Cc1cc(C(C)N2CCN(C(=O)OC(C)(C)C)CC2)c2nc(N3CCOCC3)cc(=O)n2c1] | 206 |
| 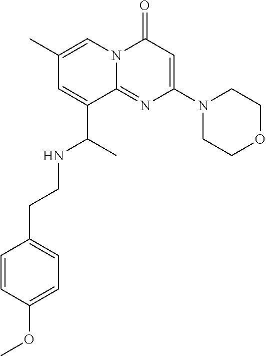 [SMILES: COc1ccc(CCNC(C)c2cc(C)cn3c(=O)cc(N4CCOCC4)nc23)cc1] | 207 |
| 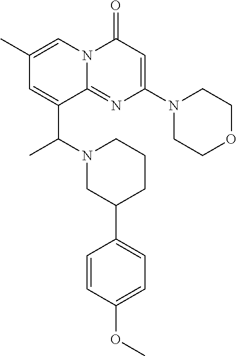 [SMILES: COc1ccc(C2CCCN(C(C)c3cc(C)cn4c(=O)cc(N5CCOCC5)nc34)C2)cc1] | 208 |
| 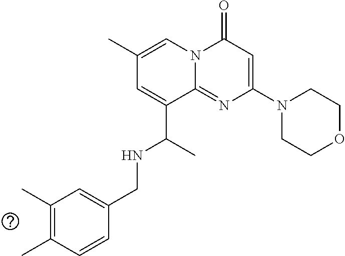 [SMILES: Cc1cc(C(C)NCc2ccc(C)c(C)c2)c2nc(N3CCOCC3)cc(=O)n2c1] | 209 |
| 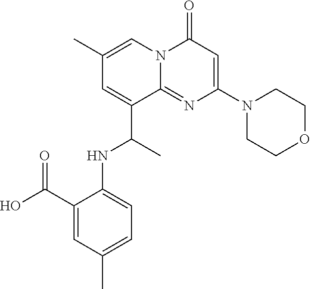 [SMILES: Cc1ccc(NC(C)c2cc(C)cn3c(=O)cc(N4CCOCC4)nc23)c(C(=O)O)c1] | 210 |
| 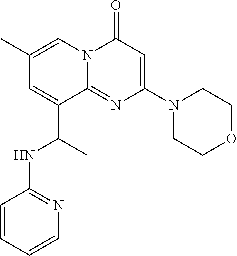 [SMILES: Cc1cc(C(C)Nc2ccccn2)c2nc(N3CCOCC3)cc(=O)n2c1] | 211 |
| 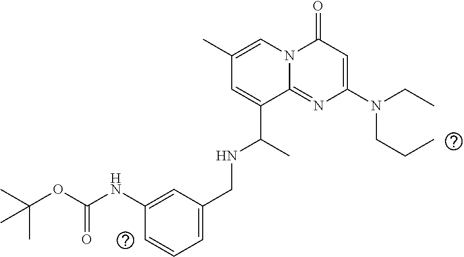 [SMILES: CCCN(CC)c1cc(=O)n2cc(C)cc(C(C)NCc3cccc(NC(=O)OC(C)(C)C)c3)c2n1] | 212 |
| 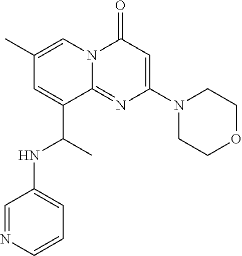 [SMILES: Cc1cc(C(C)Nc2cccnc2)c2nc(N3CCOCC3)cc(=O)n2c1] | 213 |
| 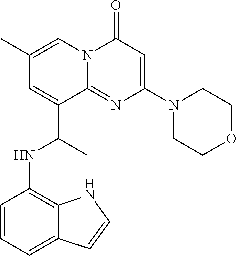 [SMILES: Cc1cc(C(C)Nc2cccc3cc[nH]c23)c2nc(N3CCOCC3)cc(=O)n2c1] | 214 |
| 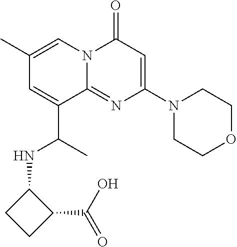 [SMILES: Cc1cc(C(C)N[C@H]2CC[C@H]2C(=O)O)c2nc(N3CCOCC3)cc(=O)n2c1] | 215 |
| 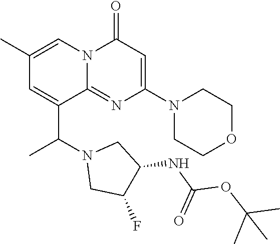 [SMILES: Cc1cc(C(C)N2C[C@@H](F)[C@@H](NC(=O)OC(C)(C)C)C2)c2nc(N3CCOCC3)cc(=O)n2c1] | 216 |
| 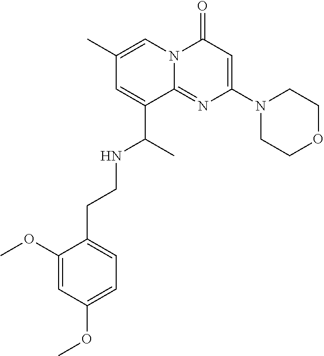 [SMILES: COc1ccc(CCNC(C)c2cc(C)cn3c(=O)cc(N4CCOCC4)nc23)c(OC)c1] | 217 |
| 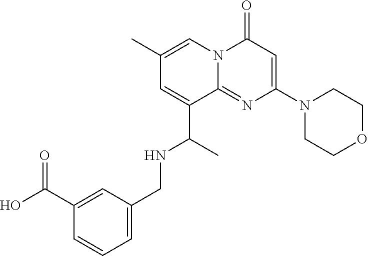 [SMILES: Cc1cc(C(C)NCc2cccc(C(=O)O)c2)c2nc(N3CCOCC3)cc(=O)n2c1] | 218 |
| 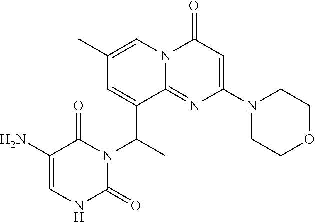 [SMILES: Cc1cc(C(C)n2c(=O)[nH]cc(N)c2=O)c2nc(N3CCOCC3)cc(=O)n2c1] | 219 |
| 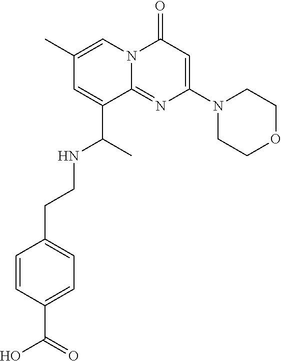 [SMILES: Cc1cc(C(C)NCCc2ccc(C(=O)O)cc2)c2nc(N3CCOCC3)cc(=O)n2c1] | 220 |
| 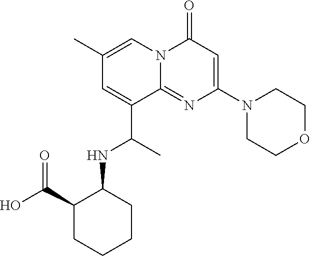 [SMILES: Cc1cc(C(C)N[C@H]2CCCC[C@H]2C(=O)O)c2nc(N3CCOCC3)cc(=O)n2c1] | 221 |
| 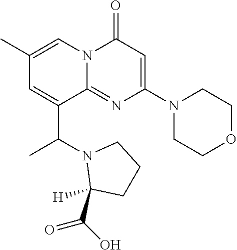 [SMILES: Cc1cc(C(C)N2CCC[C@H]2C(=O)O)c2nc(N3CCOCC3)cc(=O)n2c1] | 222 |
| 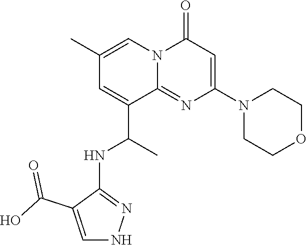 [SMILES: Cc1cc(C(C)Nc2n[nH]cc2C(=O)O)c2nc(N3CCOCC3)cc(=O)n2c1] | 223 |
| 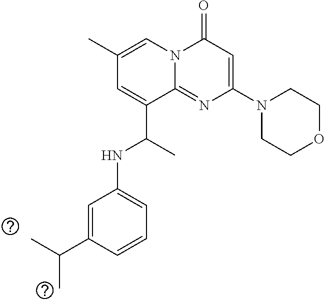 [SMILES: Cc1cc(C(C)Nc2cccc(C(C)C)c2)c2nc(N3CCOCC3)cc(=O)n2c1] | 224 |
| 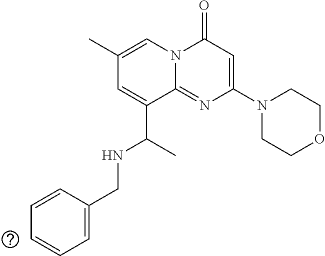 [SMILES: Cc1cc(C(C)NCc2ccccc2)c2nc(N3CCOCC3)cc(=O)n2c1] | 225 |
| 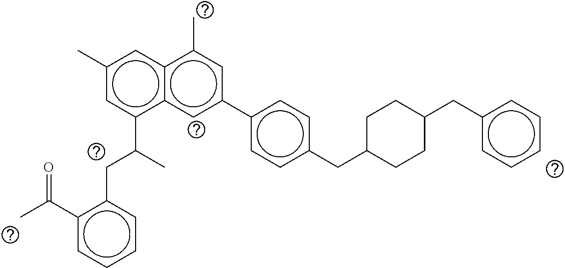 | 226 |
| 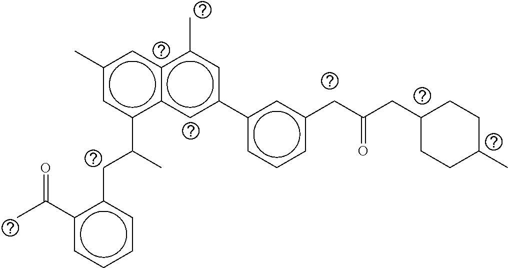 | 227 |
| 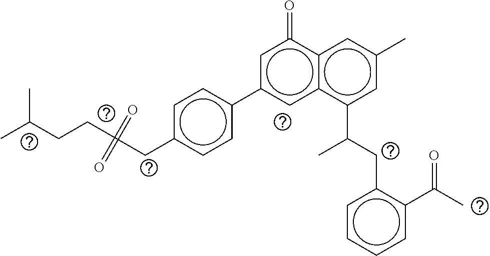 | 228 |
| 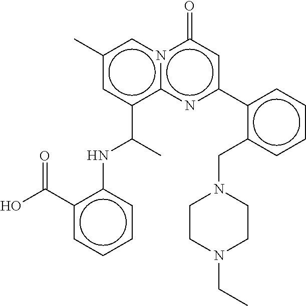 | 229 |
| 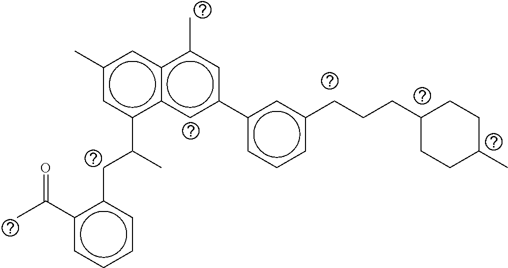 | 230 |
| 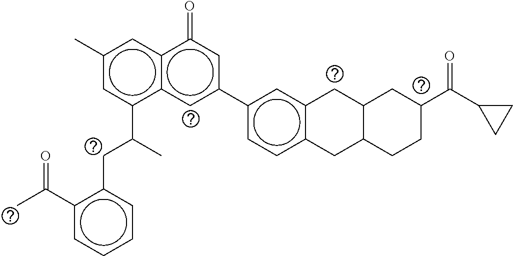 | 231 |
| 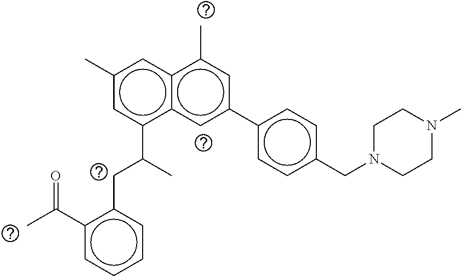 | 232 |
| 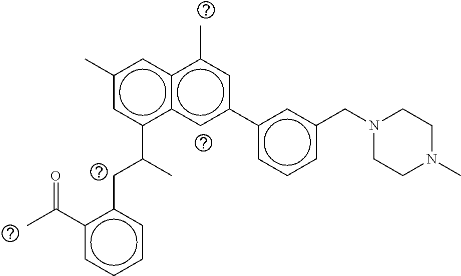 | 233 |
| 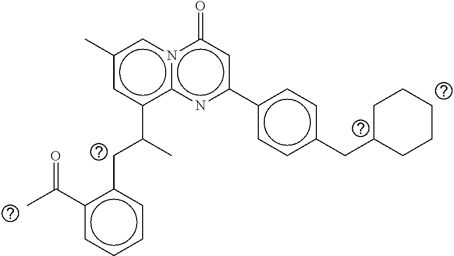 | 234 |
| 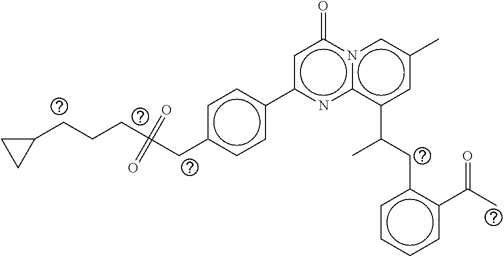 | 235 |
| 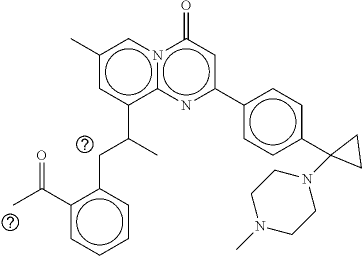 | 236 |
| 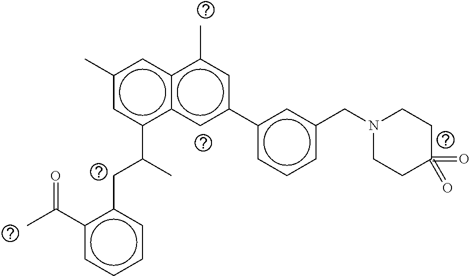 | 237 |
| 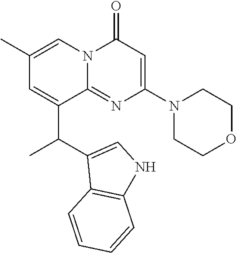 [SMILES: Cc1cc(C(C)c2c[nH]c3ccccc23)c2nc(N3CCOCC3)cc(=O)n2c1] | 238 |
| 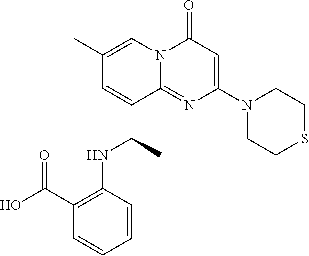 | 239 |
| 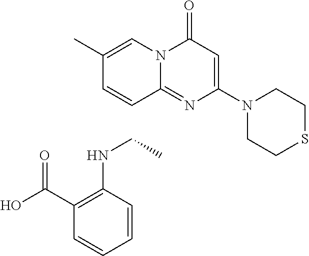 | 240 |
| 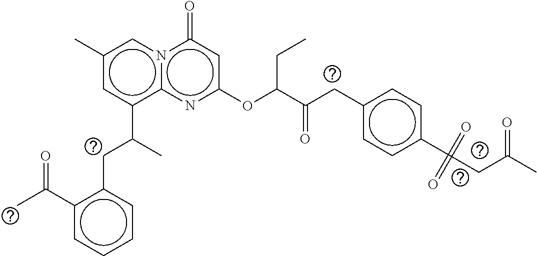 | 241 |
| 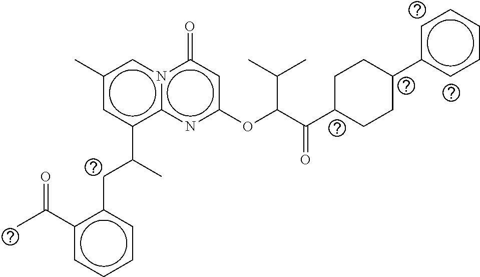 | 242 |
| 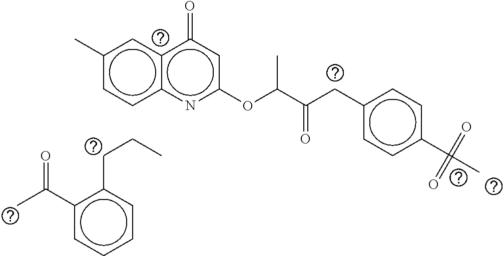 | 243 |
| 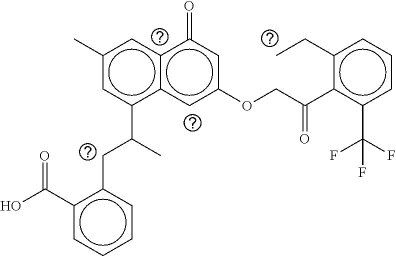 | 244 |
| 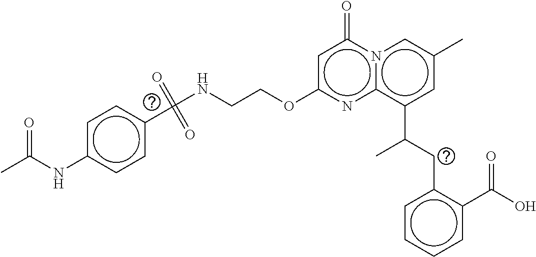 | 245 |
| 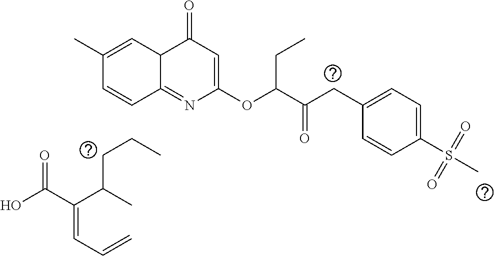 | 246 |
| 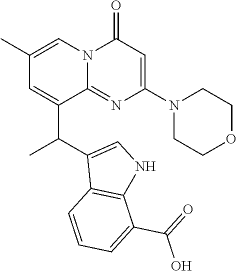 [SMILES: Cc1cc(C(C)c2c[nH]c3c(C(=O)O)cccc23)c2nc(N3CCOCC3)cc(=O)n2c1] | 247 |
| 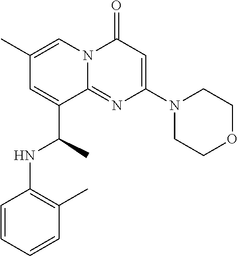 [SMILES: Cc1cc([C@@H](C)Nc2ccccc2C)c2nc(N3CCOCC3)cc(=O)n2c1] | 248 |
| 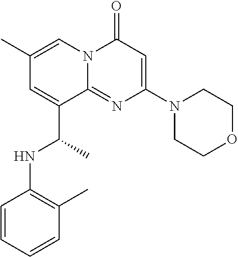 [SMILES: Cc1cc([C@H](C)Nc2ccccc2C)c2nc(N3CCOCC3)cc(=O)n2c1] | 249 |
| 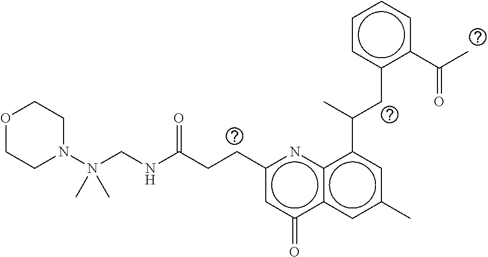 | 250 |
| 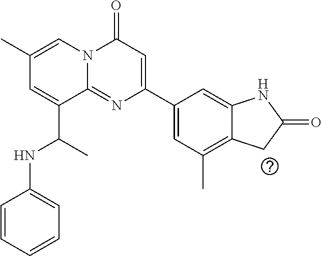 [SMILES: Cc1cc(C(C)Nc2ccccc2)c2nc(-c3cc(C)c4c(c3)NC(=O)C4)cc(=O)n2c1] | 251 |
| 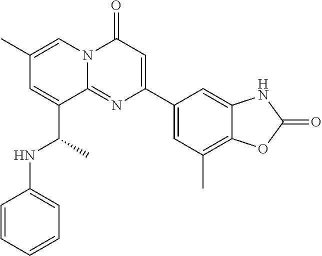 [SMILES: Cc1cc([C@H](C)Nc2ccccc2)c2nc(-c3cc(C)c4oc(=O)[nH]c4c3)cc(=O)n2c1] | 252 |
| 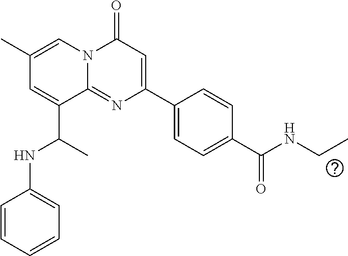 [SMILES: CCNC(=O)c1ccc(-c2cc(=O)n3cc(C)cc(C(C)Nc4ccccc4)c3n2)cc1] | 253 |
| 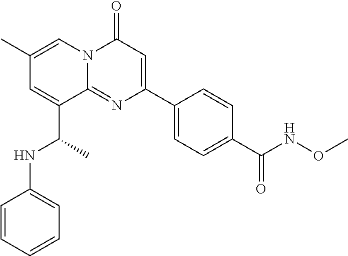 [SMILES: CONC(=O)c1ccc(-c2cc(=O)n3cc(C)cc([C@H](C)Nc4ccccc4)c3n2)cc1] | 254 |
| 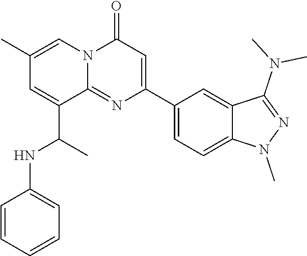 [SMILES: Cc1cc(C(C)Nc2ccccc2)c2nc(-c3ccc4c(c3)c(N(C)C)nn4C)cc(=O)n2c1] | 255 |
| 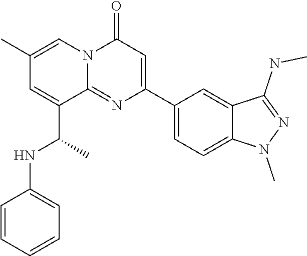 [SMILES: CNc1nn(C)c2ccc(-c3cc(=O)n4cc(C)cc([C@H](C)Nc5ccccc5)c4n3)cc12] | 256 |
| 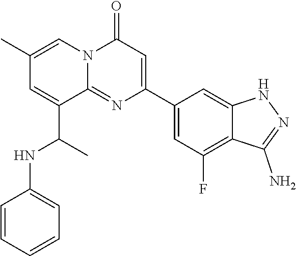 [SMILES: Cc1cc(C(C)Nc2ccccc2)c2nc(-c3cc(F)c4c(N)n[nH]c4c3)cc(=O)n2c1] | 257 |
| 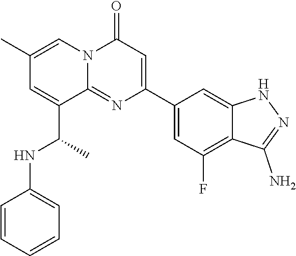 [SMILES: Cc1cc([C@H](C)Nc2ccccc2)c2nc(-c3cc(F)c4c(N)n[nH]c4c3)cc(=O)n2c1] | 258 |
| 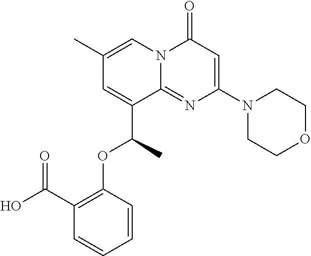 [SMILES: Cc1cc([C@@H](C)Oc2ccccc2C(=O)O)c2nc(N3CCOCC3)cc(=O)n2c1] | 259 |
| 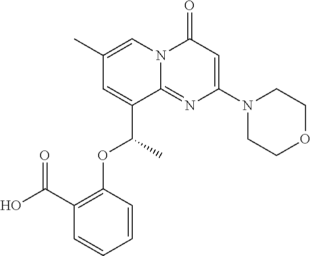 [SMILES: Cc1cc([C@H](C)Oc2ccccc2C(=O)O)c2nc(N3CCOCC3)cc(=O)n2c1] | 260 |
| 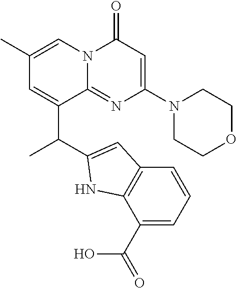 [SMILES: Cc1cc(C(C)c2cc3cccc(C(=O)O)c3[nH]2)c2nc(N3CCOCC3)cc(=O)n2c1] | 261 |
| 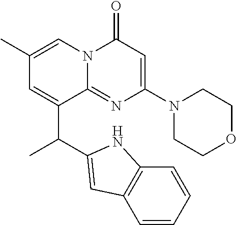 [SMILES: Cc1cc(C(C)c2cc3ccccc3[nH]2)c2nc(N3CCOCC3)cc(=O)n2c1] | 262 |
| 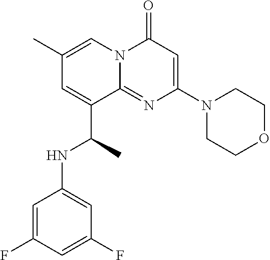 [SMILES: Cc1cc([C@@H](C)Nc2cc(F)cc(F)c2)c2nc(N3CCOCC3)cc(=O)n2c1] | 263 |
| 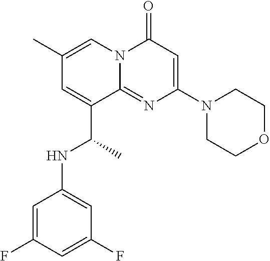 [SMILES: Cc1cc([C@H](C)Nc2cc(F)cc(F)c2)c2nc(N3CCOCC3)cc(=O)n2c1] | 264 |
| 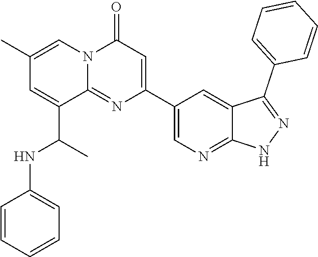 [SMILES: Cc1cc(C(C)Nc2ccccc2)c2nc(-c3cnc4[nH]nc(-c5ccccc5)c4c3)cc(=O)n2c1] | 265 |
| 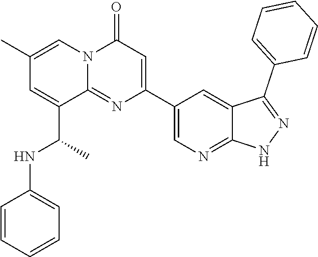 [SMILES: Cc1cc([C@H](C)Nc2ccccc2)c2nc(-c3cnc4[nH]nc(-c5ccccc5)c4c3)cc(=O)n2c1] | 266 |
| 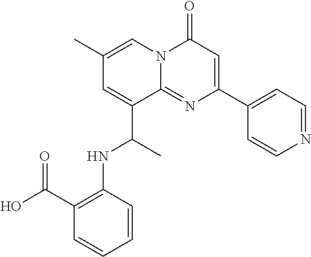 [SMILES: Cc1cc(C(C)Nc2ccccc2C(=O)O)c2nc(-c3ccncc3)cc(=O)n2c1] | 267 |
| 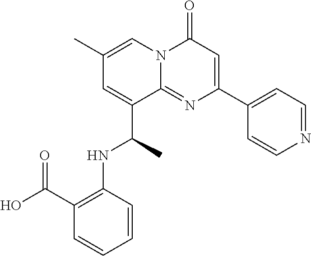 [SMILES: Cc1cc([C@@H](C)Nc2ccccc2C(=O)O)c2nc(-c3ccncc3)cc(=O)n2c1] | 268 |
| 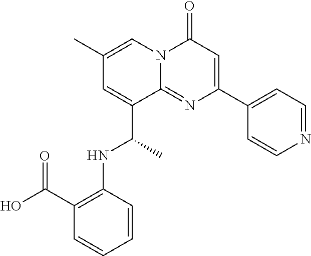 [SMILES: Cc1cc([C@H](C)Nc2ccccc2C(=O)O)c2nc(-c3ccncc3)cc(=O)n2c1] | 269 |
| 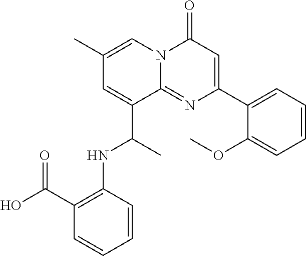 [SMILES: COc1ccccc1-c1cc(=O)n2cc(C)cc(C(C)Nc3ccccc3C(=O)O)c2n1] | 270 |
| 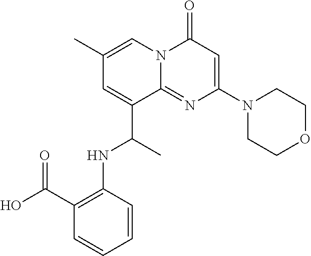 [SMILES: Cc1cc(C(C)Nc2ccccc2C(=O)O)c2nc(N3CCOCC3)cc(=O)n2c1] | 271 |
| 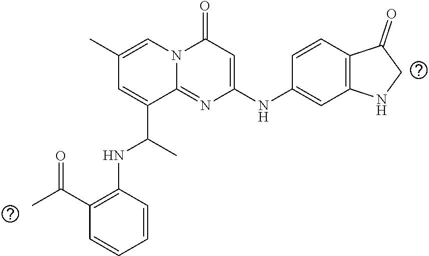 [SMILES: CC(=O)c1ccccc1NC(C)c1cc(C)cn2c(=O)cc(Nc3ccc4c(c3)NCC4=O)nc12] | 272 |
| 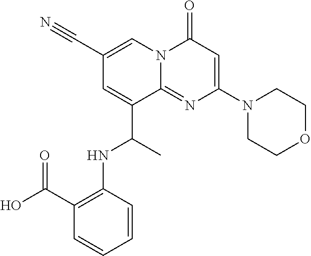 [SMILES: CC(Nc1ccccc1C(=O)O)c1cc(C#N)cn2c(=O)cc(N3CCOCC3)nc12] | 273 |
| 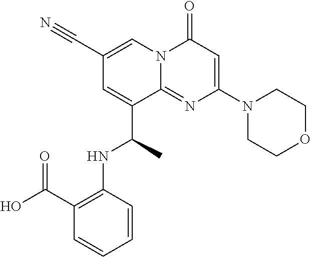 [SMILES: C[C@@H](Nc1ccccc1C(=O)O)c1cc(C#N)cn2c(=O)cc(N3CCOCC3)nc12] | 274 |
| 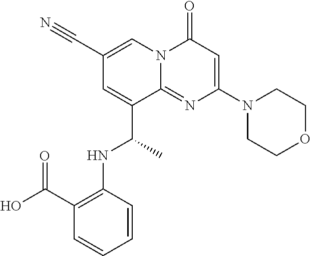 [SMILES: C[C@H](Nc1ccccc1C(=O)O)c1cc(C#N)cn2c(=O)cc(N3CCOCC3)nc12] | 275 |
| 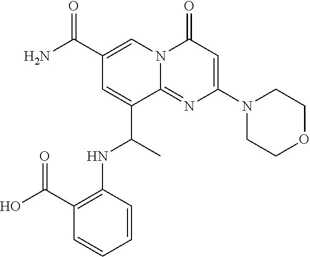 [SMILES: CC(Nc1ccccc1C(=O)O)c1cc(C(N)=O)cn2c(=O)cc(N3CCOCC3)nc12] | 276 |
| 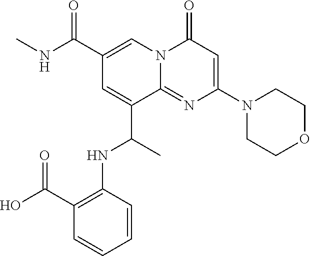 [SMILES: CNC(=O)c1cc(C(C)Nc2ccccc2C(=O)O)c2nc(N3CCOCC3)cc(=O)n2c1] | 277 |
| 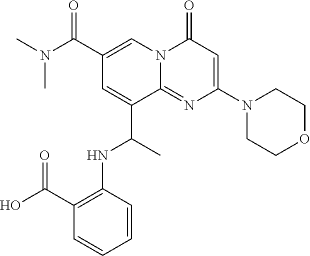 [SMILES: CC(Nc1ccccc1C(=O)O)c1cc(C(=O)N(C)C)cn2c(=O)cc(N3CCOCC3)nc12] | 278 |
| 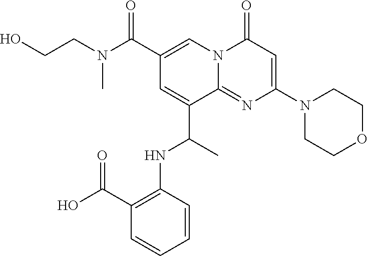 [SMILES: CC(Nc1ccccc1C(=O)O)c1cc(C(=O)N(C)CCO)cn2c(=O)cc(N3CCOCC3)nc12] | 279 |
| 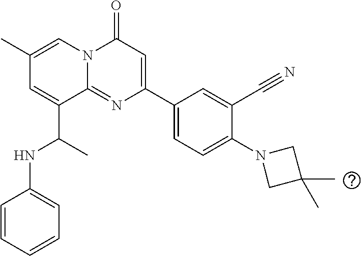 [SMILES: Cc1cc(C(C)Nc2ccccc2)c2nc(-c3ccc(N4CC(C)(C)C4)c(C#N)c3)cc(=O)n2c1] | 280 |
| 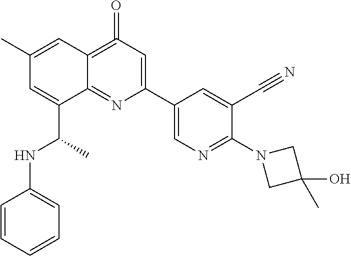 | 281 |
| 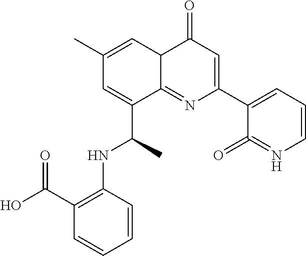 | 282 |
| 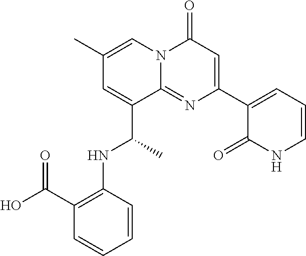 [SMILES: Cc1cc([C@H](C)Nc2ccccc2C(=O)O)c2nc(-c3ccc[nH]c3=O)cc(=O)n2c1] | 283 |
| 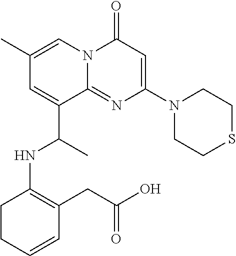 [SMILES: Cc1cc(C(C)NC2=C(CC(=O)O)C=CCC2)c2nc(N3CCSCC3)cc(=O)n2c1] | 284 |
| 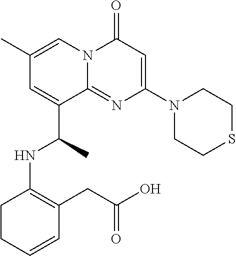 [SMILES: Cc1cc([C@@H](C)NC2=C(CC(=O)O)C=CCC2)c2nc(N3CCSCC3)cc(=O)n2c1] | 285 |
| 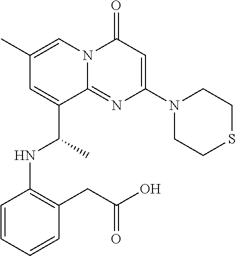 [SMILES: Cc1cc([C@H](C)Nc2ccccc2CC(=O)O)c2nc(N3CCSCC3)cc(=O)n2c1] | 286 |
| 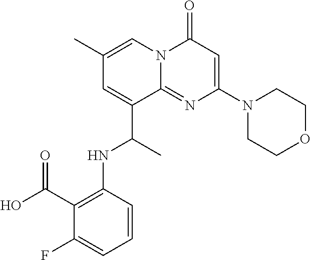 [SMILES: Cc1cc(C(C)Nc2cccc(F)c2C(=O)O)c2nc(N3CCOCC3)cc(=O)n2c1] | 287 |
| 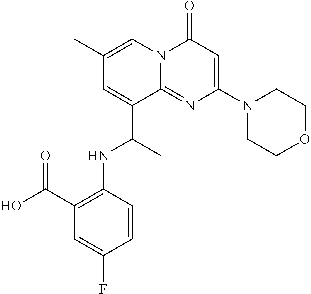 [SMILES: Cc1cc(C(C)Nc2ccc(F)cc2C(=O)O)c2nc(N3CCOCC3)cc(=O)n2c1] | 288 |
| 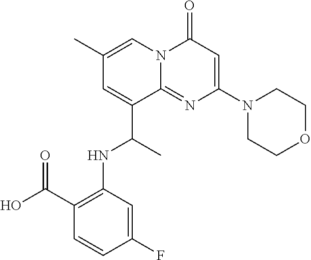 [SMILES: Cc1cc(C(C)Nc2cc(F)ccc2C(=O)O)c2nc(N3CCOCC3)cc(=O)n2c1] | 289 |
| 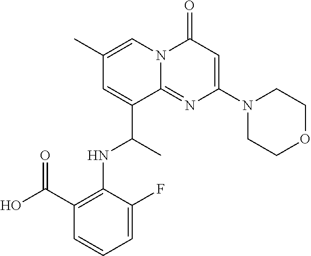 [SMILES: Cc1cc(C(C)Nc2c(F)cccc2C(=O)O)c2nc(N3CCOCC3)cc(=O)n2c1] | 290 |
| 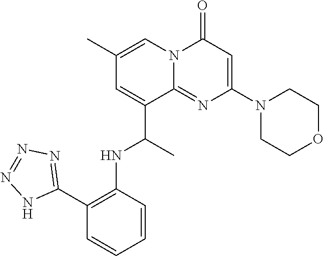 [SMILES: Cc1cc(C(C)Nc2ccccc2-c2nnn[nH]2)c2nc(N3CCOCC3)cc(=O)n2c1] | 291 |
| 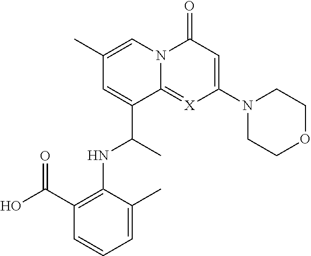 | 292 |
| 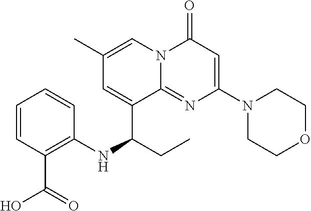 [SMILES: CC[C@@H](Nc1ccccc1C(=O)O)c1cc(C)cn2c(=O)cc(N3CCOCC3)nc12] | 293 |
| 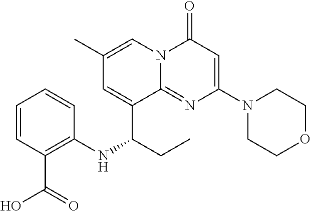 [SMILES: CC[C@H](Nc1ccccc1C(=O)O)c1cc(C)cn2c(=O)cc(N3CCOCC3)nc12] | 294 |
| 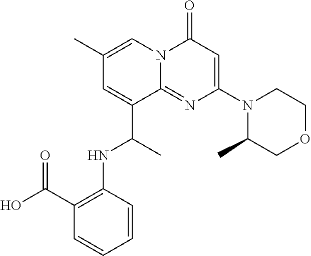 [SMILES: Cc1cc(C(C)Nc2ccccc2C(=O)O)c2nc(N3CCOC[C@H]3C)cc(=O)n2c1] | 295 |
| 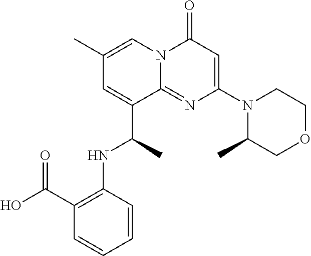 [SMILES: Cc1cc([C@@H](C)Nc2ccccc2C(=O)O)c2nc(N3CCOC[C@H]3C)cc(=O)n2c1] | 296 |
| 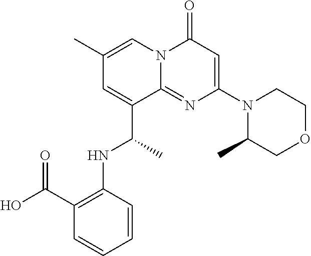 [SMILES: Cc1cc([C@H](C)Nc2ccccc2C(=O)O)c2nc(N3CCOC[C@H]3C)cc(=O)n2c1] | 297 |
| 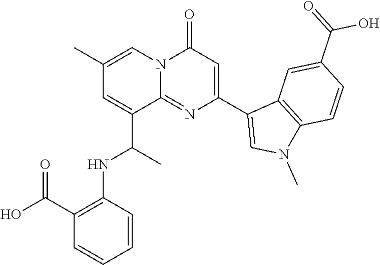 [SMILES: Cc1cc(C(C)Nc2ccccc2C(=O)O)c2nc(-c3cn(C)c4ccc(C(=O)O)cc34)cc(=O)n2c1] | 298 |
| 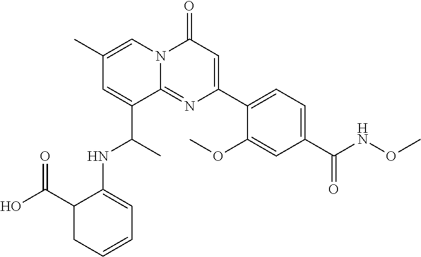 [SMILES: CONC(=O)c1ccc(-c2cc(=O)n3cc(C)cc(C(C)NC4=CC=CCC4C(=O)O)c3n2)c(OC)c1] | 299 |
| 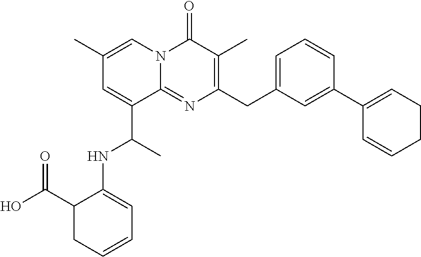 [SMILES: Cc1cc(C(C)NC2=CC=CCC2C(=O)O)c2nc(Cc3cccc(C4=CCCC=C4)c3)c(C)c(=O)n2c1] | 300 |
| 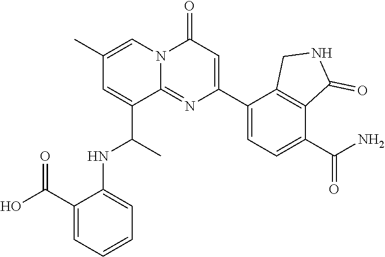 [SMILES: Cc1cc(C(C)Nc2ccccc2C(=O)O)c2nc(-c3ccc(C(N)=O)c4c3CNC4=O)cc(=O)n2c1] | 301 |
| 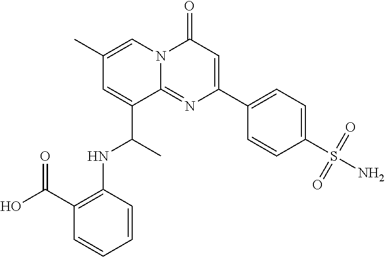 [SMILES: Cc1cc(C(C)Nc2ccccc2C(=O)O)c2nc(-c3ccc(S(N)(=O)=O)cc3)cc(=O)n2c1] | 302 |
| 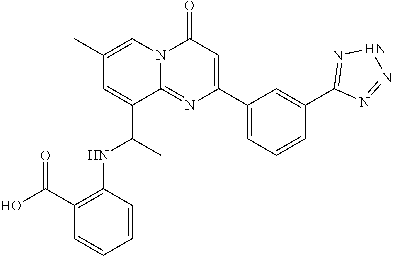 [SMILES: Cc1cc(C(C)Nc2ccccc2C(=O)O)c2nc(-c3cccc(-c4nn[nH]n4)c3)cc(=O)n2c1] | 303 |
| 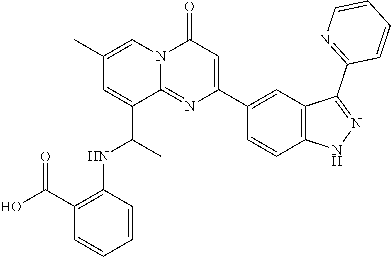 [SMILES: Cc1cc(C(C)Nc2ccccc2C(=O)O)c2nc(-c3ccc4[nH]nc(-c5ccccn5)c4c3)cc(=O)n2c1] | 304 |
| 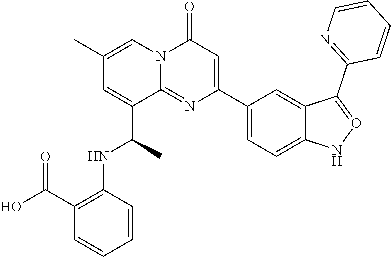 | 305 |
| 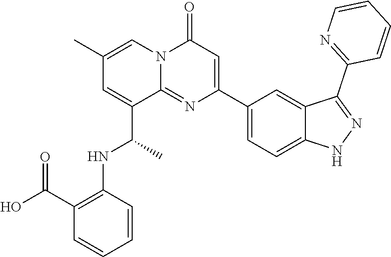 [SMILES: Cc1cc([C@H](C)Nc2ccccc2C(=O)O)c2nc(-c3ccc4[nH]nc(-c5ccccn5)c4c3)cc(=O)n2c1] | 306 |
| 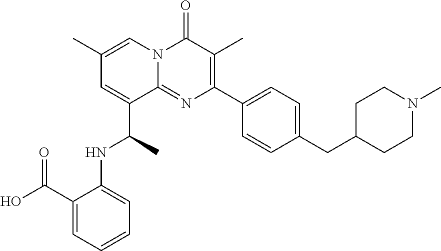 [SMILES: Cc1cc([C@@H](C)Nc2ccccc2C(=O)O)c2nc(-c3ccc(CC4CCN(C)CC4)cc3)c(C)c(=O)n2c1] | 307 |
| 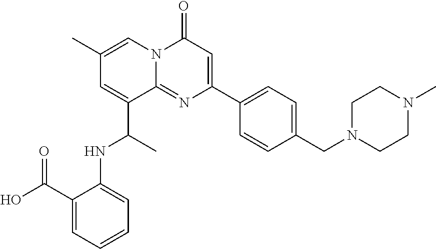 [SMILES: Cc1cc(C(C)Nc2ccccc2C(=O)O)c2nc(-c3ccc(CN4CCN(C)CC4)cc3)cc(=O)n2c1] | 308 |
| 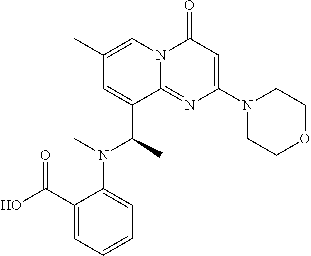 [SMILES: Cc1cc([C@@H](C)N(C)c2ccccc2C(=O)O)c2nc(N3CCOCC3)cc(=O)n2c1] | 309 |
| 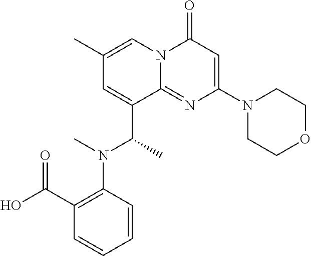 [SMILES: Cc1cc([C@H](C)N(C)c2ccccc2C(=O)O)c2nc(N3CCOCC3)cc(=O)n2c1] | 310 |
| 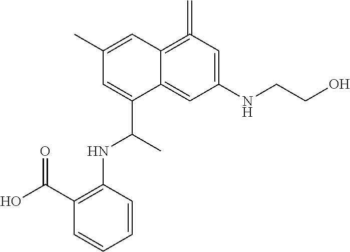 | 311 |
| 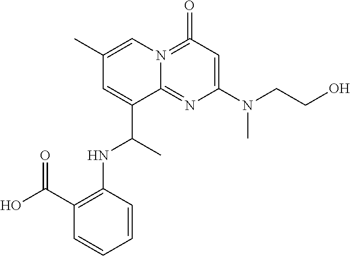 | 312 |
| 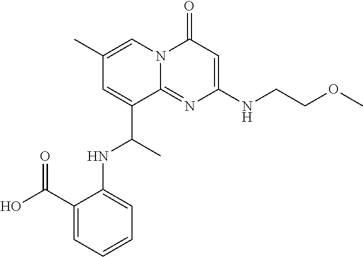 [SMILES: COCCNc1cc(=O)n2cc(C)cc(C(C)Nc3ccccc3C(=O)O)c2n1] | 313 |
| 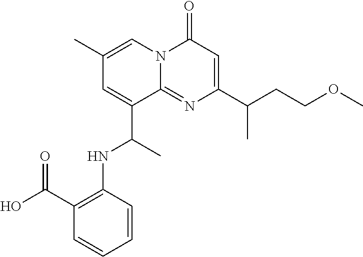 [SMILES: COCCC(C)c1cc(=O)n2cc(C)cc(C(C)Nc3ccccc3C(=O)O)c2n1] | 314 |
| 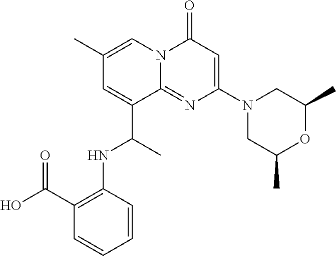 [SMILES: Cc1cc(C(C)Nc2ccccc2C(=O)O)c2nc(N3C[C@@H](C)O[C@@H](C)C3)cc(=O)n2c1] | 315 |
| 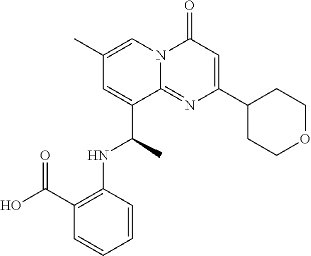 [SMILES: Cc1cc([C@@H](C)Nc2ccccc2C(=O)O)c2nc(C3CCOCC3)cc(=O)n2c1] | 316 |
| 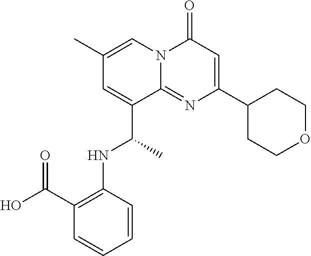 [SMILES: Cc1cc([C@H](C)Nc2ccccc2C(=O)O)c2nc(C3CCOCC3)cc(=O)n2c1] | 317 |
| 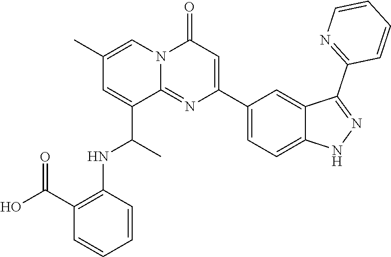 [SMILES: Cc1cc(C(C)Nc2ccccc2C(=O)O)c2nc(-c3ccc4[nH]nc(-c5ccccn5)c4c3)cc(=O)n2c1] | 318 |
| 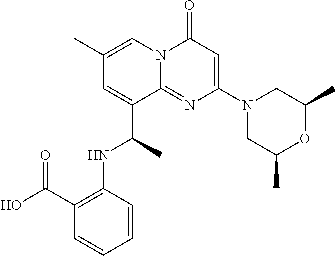 [SMILES: Cc1cc([C@@H](C)Nc2ccccc2C(=O)O)c2nc(N3C[C@@H](C)O[C@@H](C)C3)cc(=O)n2c1] | 319 |
| 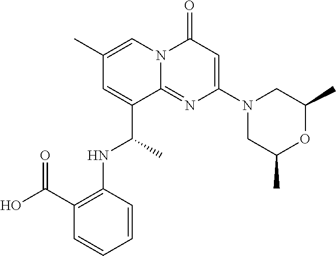 [SMILES: Cc1cc([C@H](C)Nc2ccccc2C(=O)O)c2nc(N3C[C@@H](C)O[C@@H](C)C3)cc(=O)n2c1] | 320 |
| 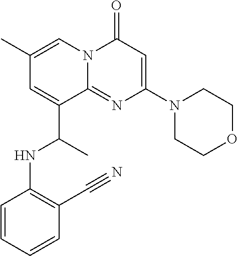 [SMILES: Cc1cc(C(C)Nc2ccccc2C#N)c2nc(N3CCOCC3)cc(=O)n2c1] | 321 |
| 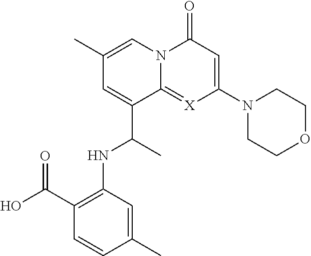 | 322 |
| 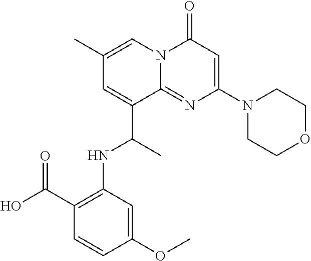 [SMILES: COc1ccc(C(=O)O)c(NC(C)c2cc(C)cn3c(=O)cc(N4CCOCC4)nc23)c1] | 323 |
| 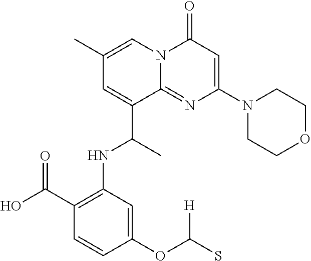 [SMILES: Cc1cc(C(C)Nc2cc(OCS)ccc2C(=O)O)c2nc(N3CCOCC3)cc(=O)n2c1] | 324 |
| 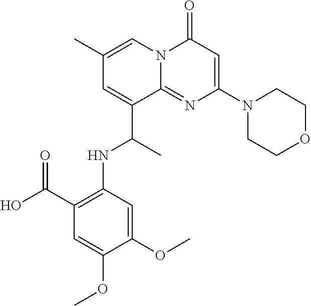 [SMILES: COc1cc(NC(C)c2cc(C)cn3c(=O)cc(N4CCOCC4)nc23)c(C(=O)O)cc1OC] | 325 |
| 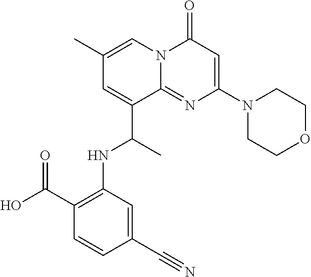 [SMILES: Cc1cc(C(C)Nc2cc(C#N)ccc2C(=O)O)c2nc(N3CCOCC3)cc(=O)n2c1] | 326 |
| 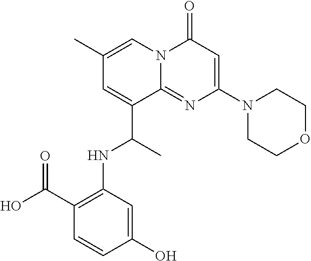 [SMILES: Cc1cc(C(C)Nc2cc(O)ccc2C(=O)O)c2nc(N3CCOCC3)cc(=O)n2c1] | 327 |
| 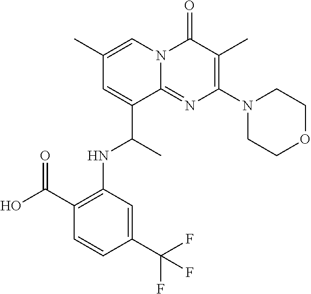 [SMILES: Cc1cc(C(C)Nc2cc(C(F)(F)F)ccc2C(=O)O)c2nc(N3CCOCC3)c(C)c(=O)n2c1] | 328 |
| 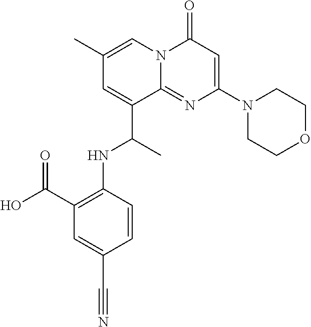 [SMILES: Cc1cc(C(C)Nc2ccc(C#N)cc2C(=O)O)c2nc(N3CCOCC3)cc(=O)n2c1] | 329 |
| 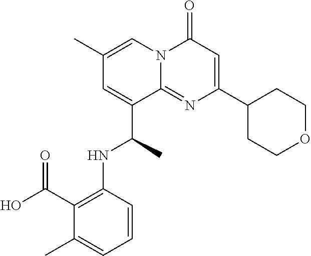 [SMILES: Cc1cc([C@@H](C)Nc2cccc(C)c2C(=O)O)c2nc(C3CCOCC3)cc(=O)n2c1] | 330 |
| 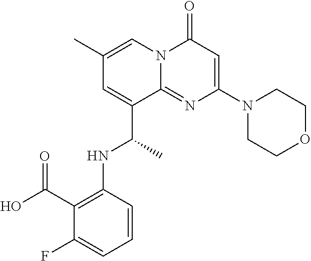 [SMILES: Cc1cc([C@H](C)Nc2cccc(F)c2C(=O)O)c2nc(N3CCOCC3)cc(=O)n2c1] | 331 |
| 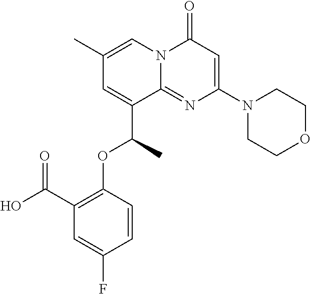 [SMILES: Cc1cc([C@@H](C)Oc2ccc(F)cc2C(=O)O)c2nc(N3CCOCC3)cc(=O)n2c1] | 332 |
| 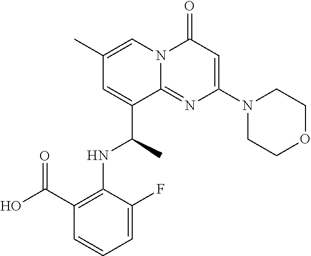 [SMILES: Cc1cc([C@@H](C)Nc2c(F)cccc2C(=O)O)c2nc(N3CCOCC3)cc(=O)n2c1] | 333 |
| 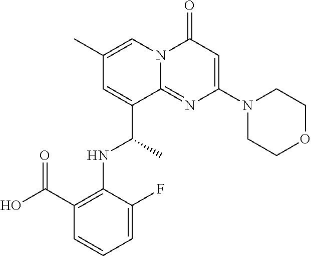 [SMILES: Cc1cc([C@H](C)Nc2c(F)cccc2C(=O)O)c2nc(N3CCOCC3)cc(=O)n2c1] | 334 |
| 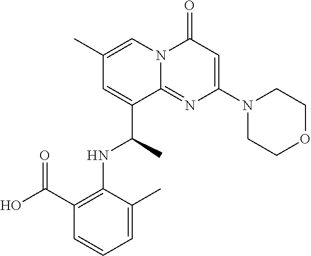 [SMILES: Cc1cc([C@@H](C)Nc2c(C)cccc2C(=O)O)c2nc(N3CCOCC3)cc(=O)n2c1] | 335 |
| 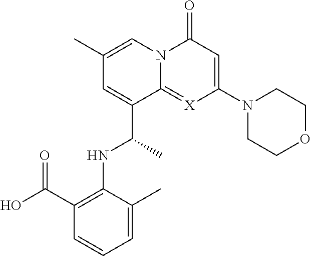 | 336 |
| 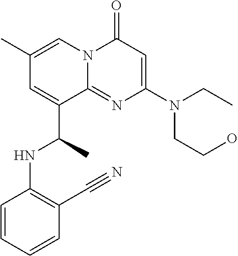 [SMILES: CCN(CCO)c1cc(=O)n2cc(C)cc([C@@H](C)Nc3ccccc3C#N)c2n1] | 337 |
| 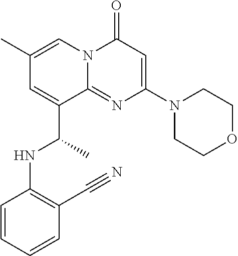 [SMILES: Cc1cc([C@H](C)Nc2ccccc2C#N)c2nc(N3CCOCC3)cc(=O)n2c1] | 338 |
| 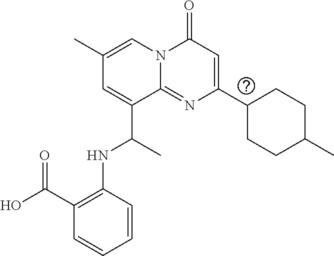 [SMILES: Cc1cc(C(C)Nc2ccccc2C(=O)O)c2nc(C3CCC(C)CC3)cc(=O)n2c1] | 339 |
| 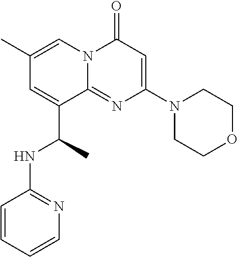 [SMILES: Cc1cc([C@@H](C)Nc2ccccn2)c2nc(N3CCOCC3)cc(=O)n2c1] | 340 |
| 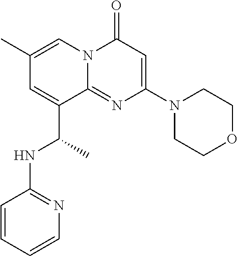 [SMILES: Cc1cc([C@H](C)Nc2ccccn2)c2nc(N3CCOCC3)cc(=O)n2c1] | 341 |
| 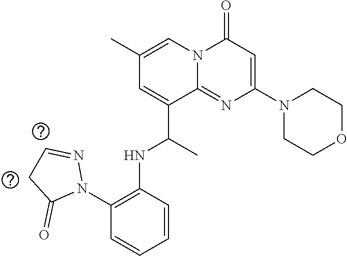 [SMILES: Cc1cc(C(C)Nc2ccccc2N2N=CCC2=O)c2nc(N3CCOCC3)cc(=O)n2c1] | 342 |
| 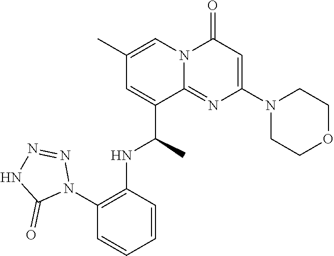 [SMILES: Cc1cc([C@@H](C)Nc2ccccc2-n2nn[nH]c2=O)c2nc(N3CCOCC3)cc(=O)n2c1] | 343 |
| 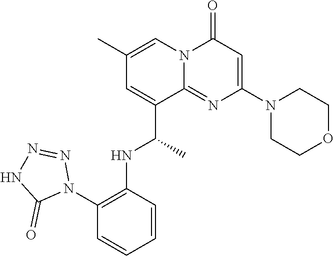 [SMILES: Cc1cc([C@H](C)Nc2ccccc2-n2nn[nH]c2=O)c2nc(N3CCOCC3)cc(=O)n2c1] | 344 |
| 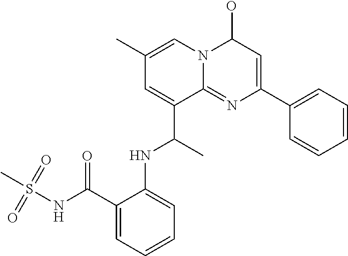 [SMILES: CC1=CN2C(=NC(c3ccccc3)=CC2O)C(C(C)Nc2ccccc2C(=O)NS(C)(=O)=O)=C1] | 345 |
| 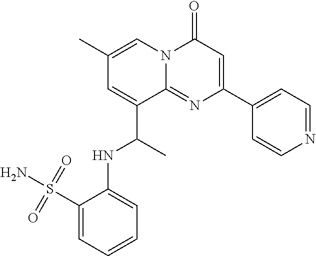 [SMILES: Cc1cc(C(C)Nc2ccccc2S(N)(=O)=O)c2nc(-c3ccncc3)cc(=O)n2c1] | 346 |
| 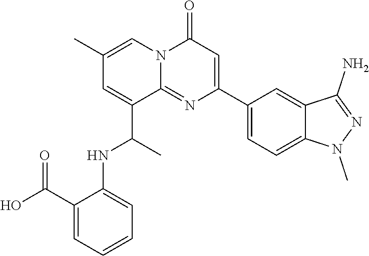 [SMILES: Cc1cc(C(C)Nc2ccccc2C(=O)O)c2nc(-c3ccc4c(c3)c(N)nn4C)cc(=O)n2c1] | 347 |
| 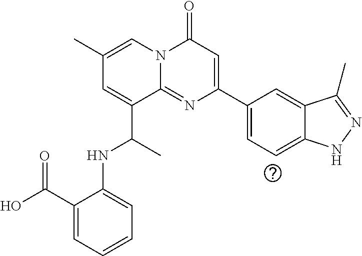 [SMILES: Cc1cc(C(C)Nc2ccccc2C(=O)O)c2nc(-c3ccc4[nH]nc(C)c4c3)cc(=O)n2c1] | 348 |
| 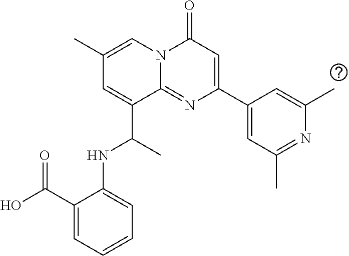 [SMILES: Cc1cc(C(C)Nc2ccccc2C(=O)O)c2nc(-c3cc(C)nc(C)c3)cc(=O)n2c1] | 349 |
| 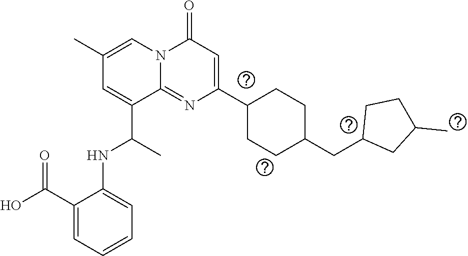 [SMILES: Cc1cc(C(C)Nc2ccccc2C(=O)O)c2nc(C3CCC(CC4CCC(C)C4)CC3)cc(=O)n2c1] | 350 |
| 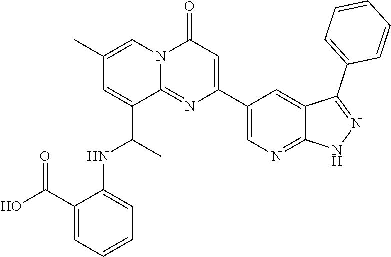 [SMILES: Cc1cc(C(C)Nc2ccccc2C(=O)O)c2nc(-c3cnc4[nH]nc(-c5ccccc5)c4c3)cc(=O)n2c1] | 351 |
| 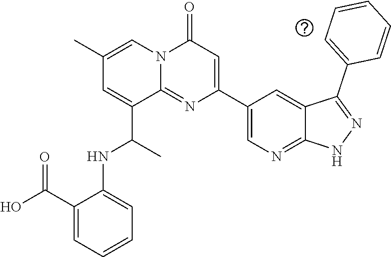 [SMILES: Cc1cc(C(C)Nc2ccccc2C(=O)O)c2nc(-c3cnc4[nH]nc(-c5ccccc5)c4c3)cc(=O)n2c1] | 352 |
| 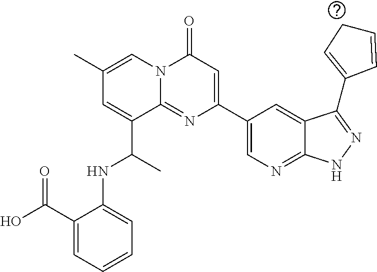 [SMILES: Cc1cc(C(C)Nc2ccccc2C(=O)O)c2nc(-c3cnc4[nH]nc(C5=CCC=C5)c4c3)cc(=O)n2c1] | 353 |
| 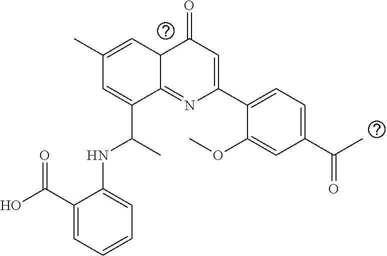 [SMILES: COc1cc(C(C)=O)ccc1C1=CC(=O)C2C=C(C)C=C(C(C)Nc3ccccc3C(=O)O)C2=N1] | 354 |
| 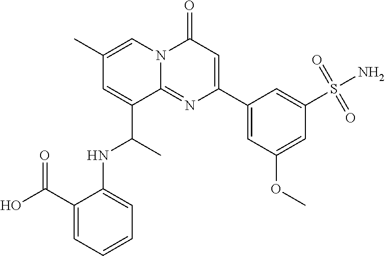 [SMILES: COc1cc(-c2cc(=O)n3cc(C)cc(C(C)Nc4ccccc4C(=O)O)c3n2)cc(S(N)(=O)=O)c1] | 355 |
| 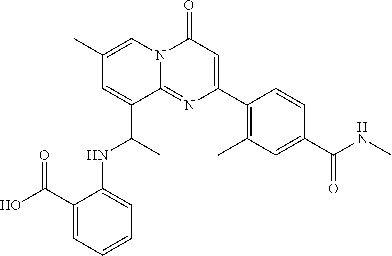 [SMILES: CNC(=O)c1ccc(-c2cc(=O)n3cc(C)cc(C(C)Nc4ccccc4C(=O)O)c3n2)c(C)c1] | 356 |
| 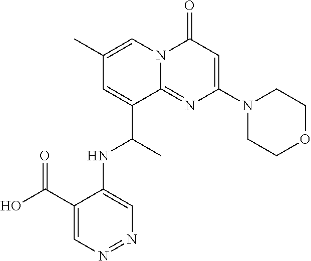 [SMILES: Cc1cc(C(C)Nc2cnncc2C(=O)O)c2nc(N3CCOCC3)cc(=O)n2c1] | 357 |
| 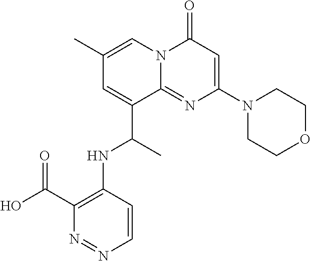 [SMILES: Cc1cc(C(C)Nc2ccnnc2C(=O)O)c2nc(N3CCOCC3)cc(=O)n2c1] | 358 |
| 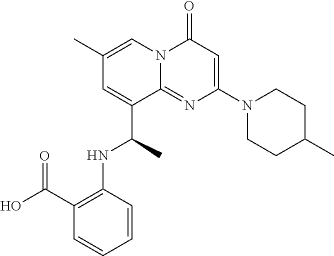 [SMILES: Cc1cc([C@@H](C)Nc2ccccc2C(=O)O)c2nc(N3CCC(C)CC3)cc(=O)n2c1] | 359 |
| 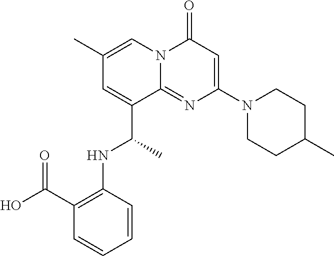 [SMILES: Cc1cc([C@H](C)Nc2ccccc2C(=O)O)c2nc(N3CCC(C)CC3)cc(=O)n2c1] | 360 |
| 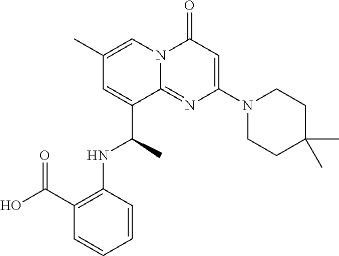 [SMILES: Cc1cc([C@@H](C)Nc2ccccc2C(=O)O)c2nc(N3CCC(C)(C)CC3)cc(=O)n2c1] | 361 |
| 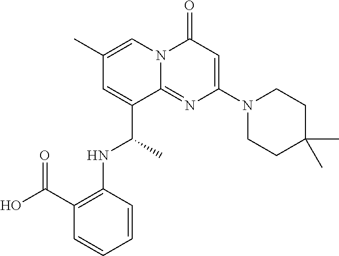 [SMILES: Cc1cc([C@H](C)Nc2ccccc2C(=O)O)c2nc(N3CCC(C)(C)CC3)cc(=O)n2c1] | 362 |
| 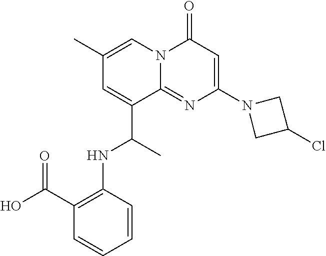 [SMILES: Cc1cc(C(C)Nc2ccccc2C(=O)O)c2nc(N3CC(Cl)C3)cc(=O)n2c1] | 363 |
| 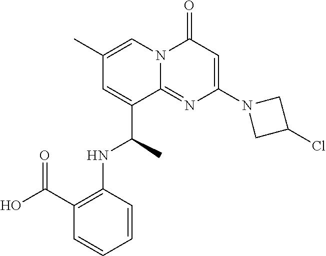 [SMILES: Cc1cc([C@@H](C)Nc2ccccc2C(=O)O)c2nc(N3CC(Cl)C3)cc(=O)n2c1] | 364 |
| 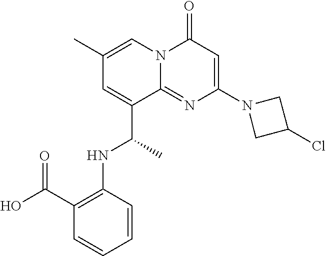 [SMILES: Cc1cc([C@H](C)Nc2ccccc2C(=O)O)c2nc(N3CC(Cl)C3)cc(=O)n2c1] | 365 |
| 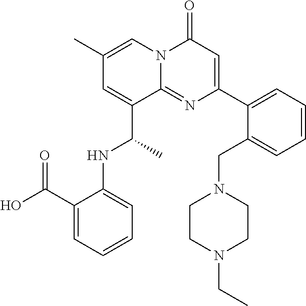 [SMILES: CCN1CCN(Cc2ccccc2-c2cc(=O)n3cc(C)cc([C@H](C)Nc4ccccc4C(=O)O)c3n2)CC1] | 366 |
| 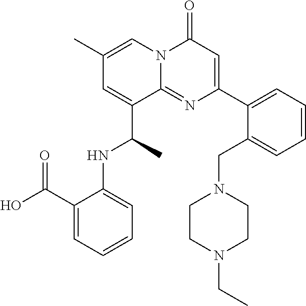 [SMILES: CCN1CCN(Cc2ccccc2-c2cc(=O)n3cc(C)cc([C@@H](C)Nc4ccccc4C(=O)O)c3n2)CC1] | 367 |
| 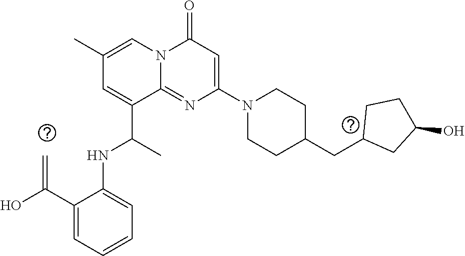 [SMILES: C=C(O)c1ccccc1NC(C)c1cc(C)cn2c(=O)cc(N3CCC(CC4CC[C@@H](O)C4)CC3)nc12] | 368 |
| 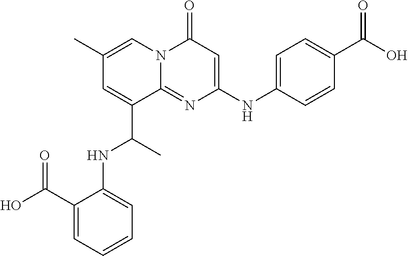 [SMILES: Cc1cc(C(C)Nc2ccccc2C(=O)O)c2nc(Nc3ccc(C(=O)O)cc3)cc(=O)n2c1] | 369 |
| 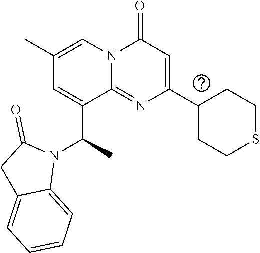 [SMILES: Cc1cc([C@@H](C)N2C(=O)Cc3ccccc32)c2nc(C3CCSCC3)cc(=O)n2c1] | 370 |
| 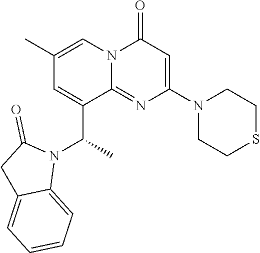 [SMILES: Cc1cc([C@H](C)N2C(=O)Cc3ccccc32)c2nc(N3CCSCC3)cc(=O)n2c1] | 371 |
| 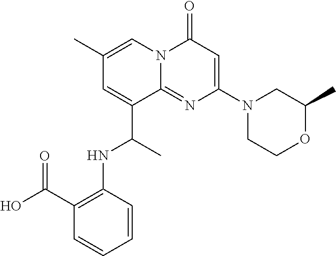 [SMILES: Cc1cc(C(C)Nc2ccccc2C(=O)O)c2nc(N3CCO[C@H](C)C3)cc(=O)n2c1] | 372 |
| 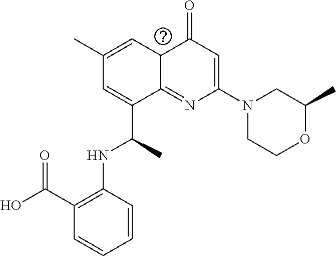 [SMILES: CC1=CC2C(=O)C=C(N3CCO[C@H](C)C3)N=C2C([C@@H](C)Nc2ccccc2C(=O)O)=C1] | 373 |
| 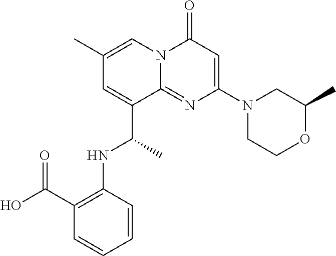 [SMILES: Cc1cc([C@H](C)Nc2ccccc2C(=O)O)c2nc(N3CCO[C@H](C)C3)cc(=O)n2c1] | 374 |
| 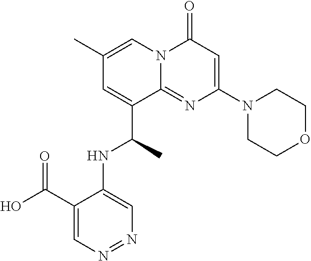 [SMILES: Cc1cc([C@@H](C)Nc2cnncc2C(=O)O)c2nc(N3CCOCC3)cc(=O)n2c1] | 375 |
| 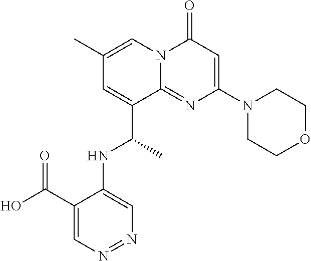 [SMILES: Cc1cc([C@H](C)Nc2cnncc2C(=O)O)c2nc(N3CCOCC3)cc(=O)n2c1] | 376 |
| 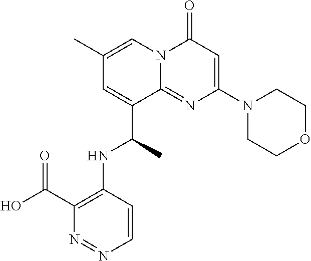 [SMILES: Cc1cc([C@@H](C)Nc2ccnnc2C(=O)O)c2nc(N3CCOCC3)cc(=O)n2c1] | 377 |
| 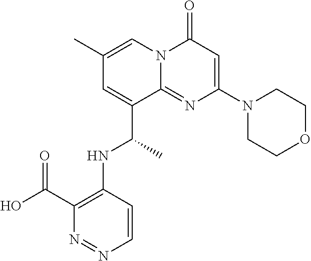 [SMILES: Cc1cc([C@H](C)Nc2ccnnc2C(=O)O)c2nc(N3CCOCC3)cc(=O)n2c1] | 378 |
| 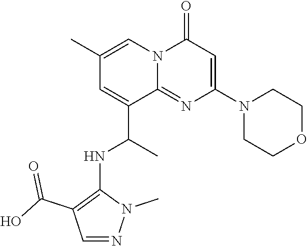 [SMILES: Cc1cc(C(C)Nc2c(C(=O)O)cnn2C)c2nc(N3CCOCC3)cc(=O)n2c1] | 379 |
| 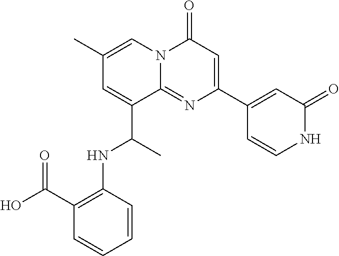 [SMILES: Cc1cc(C(C)Nc2ccccc2C(=O)O)c2nc(-c3cc[nH]c(=O)c3)cc(=O)n2c1] | 380 |
| 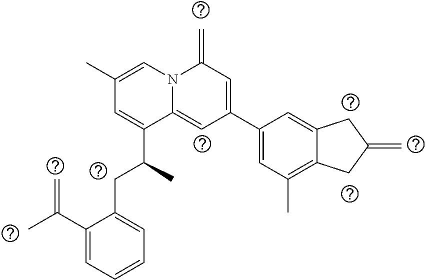 [SMILES: C=C1Cc2cc(C3=CC(=C)N4C=C(C)C=C([C@@H](C)Cc5ccccc5C(=C)C)C4=C3)cc(C)c2C1] | 381 |
| 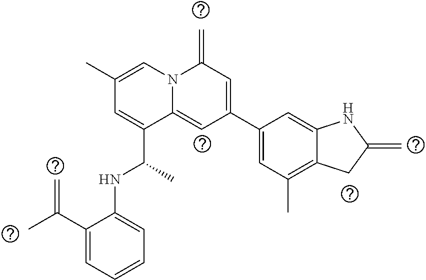 [SMILES: C=C1Cc2c(C)cc(C3=CC(=C)N4C=C(C)C=C([C@H](C)Nc5ccccc5C(=C)C)C4=C3)cc2N1] | 382 |
| 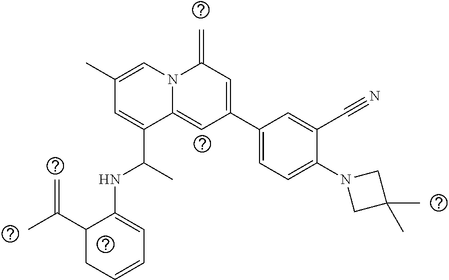 [SMILES: C=C(C)C1CC=CC=C1NC(C)C1=CC(C)=CN2C(=C)C=C(c3ccc(N4CC(C)(C)C4)c(C#N)c3)C=C12] | 383 |
| 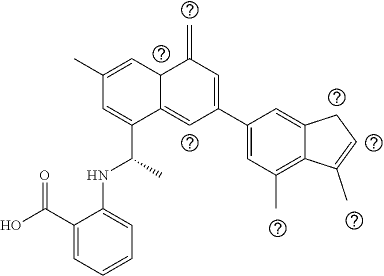 [SMILES: C=C1C=C(c2cc(C)c3c(c2)CC=C3C)C=C2C([C@H](C)Nc3ccccc3C(=O)O)=CC(C)=CC12] | 384 |
| 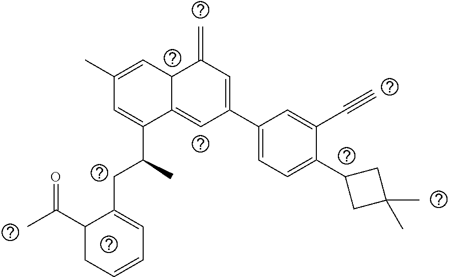 [SMILES: C#Cc1cc(C2=CC(=C)C3C=C(C)C=C([C@@H](C)CC4=CC=CCC4C(C)=O)C3=C2)ccc1C1CC(C)(C)C1] | 385 |
| 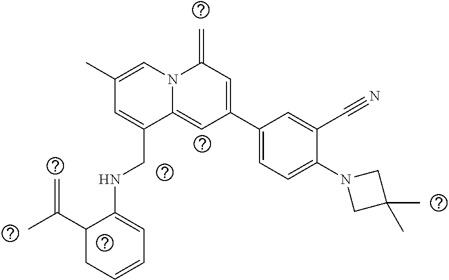 [SMILES: C=C(C)C1CC=CC=C1NCC1=CC(C)=CN2C(=C)C=C(c3ccc(N4CC(C)(C)C4)c(C#N)c3)C=C12] | 386 |
| 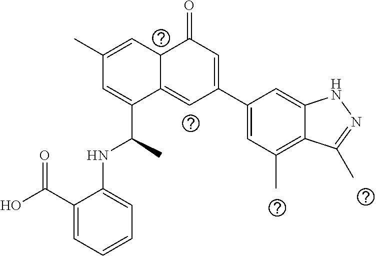 [SMILES: CC1=CC2C(=O)C=C(c3cc(C)c4c(C)n[nH]c4c3)C=C2C([C@@H](C)Nc2ccccc2C(=O)O)=C1] | 387 |
| 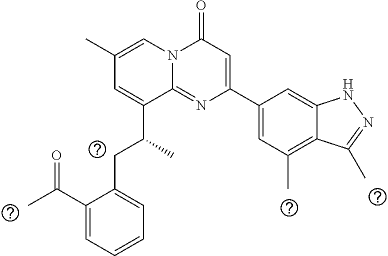 [SMILES: CC(=O)c1ccccc1C[C@@H](C)c1cc(C)cn2c(=O)cc(-c3cc(C)c4c(C)n[nH]c4c3)nc12] | 388 |
| 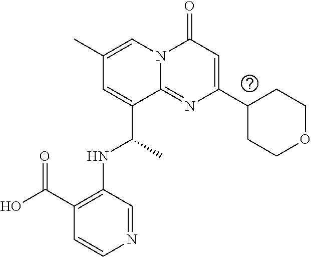 [SMILES: Cc1cc([C@H](C)Nc2cnccc2C(=O)O)c2nc(C3CCOCC3)cc(=O)n2c1] | 389 |
| 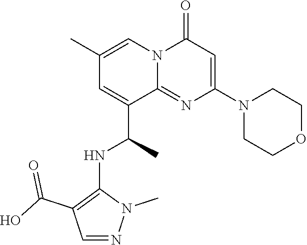 [SMILES: Cc1cc([C@@H](C)Nc2c(C(=O)O)cnn2C)c2nc(N3CCOCC3)cc(=O)n2c1] | 390 |
| 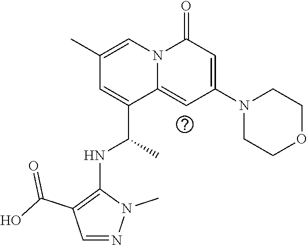 [SMILES: Cc1cc([C@H](C)Nc2c(C(=O)O)cnn2C)c2cc(N3CCOCC3)cc(=O)n2c1] | 391 |
| 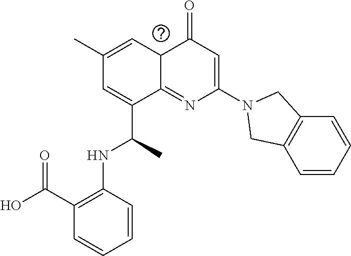 [SMILES: CC1=CC2C(=O)C=C(N3Cc4ccccc4C3)N=C2C([C@@H](C)Nc2ccccc2C(=O)O)=C1] | 392 |
| 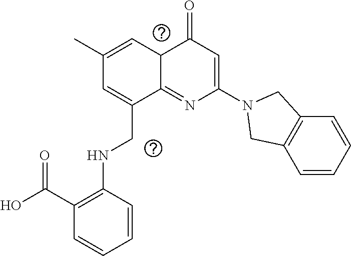 [SMILES: CC1=CC2C(=O)C=C(N3Cc4ccccc4C3)N=C2C(CNc2ccccc2C(=O)O)=C1] | 393 |
| 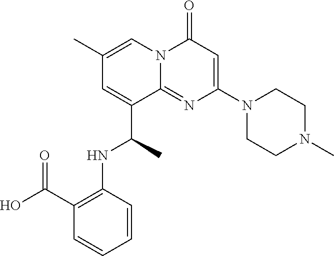 [SMILES: Cc1cc([C@@H](C)Nc2ccccc2C(=O)O)c2nc(N3CCN(C)CC3)cc(=O)n2c1] | 394 |
| 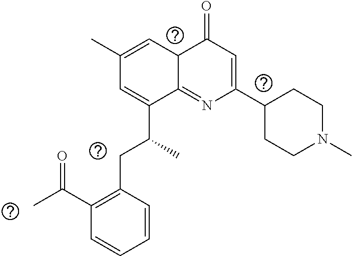 [SMILES: CC(=O)c1ccccc1C[C@@H](C)C1=CC(C)=CC2C(=O)C=C(C3CCN(C)CC3)N=C12] | 395 |
| 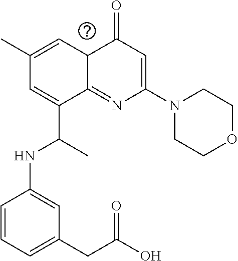 [SMILES: CC1=CC2C(=O)C=C(N3CCOCC3)N=C2C(C(C)Nc2cccc(CC(=O)O)c2)=C1] | 396 |
| 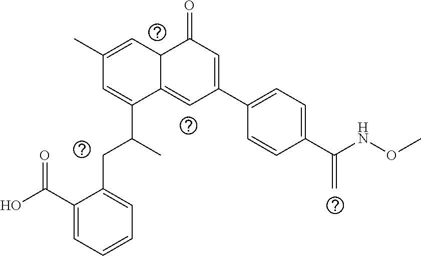 [SMILES: C=C(NOC)c1ccc(C2=CC(=O)C3C=C(C)C=C(C(C)Cc4ccccc4C(=O)O)C3=C2)cc1] | 397 |
| 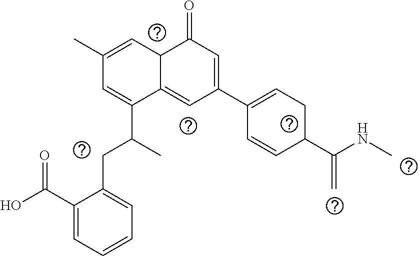 [SMILES: C=C(NC)C1C=CC(C2=CC(=O)C3C=C(C)C=C(C(C)Cc4ccccc4C(=O)O)C3=C2)=CC1] | 398 |
| 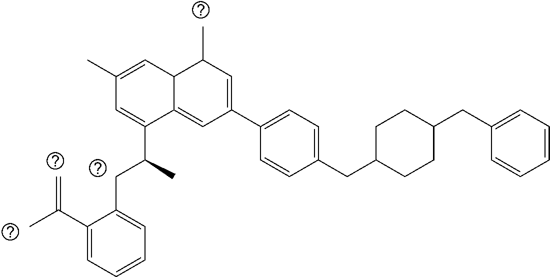 [SMILES: C=C(C)c1ccccc1C[C@H](C)C1=CC(C)=CC2C1=CC(c1ccc(CC3CCC(Cc4ccccc4)CC3)cc1)=CC2C] | 399 |
| 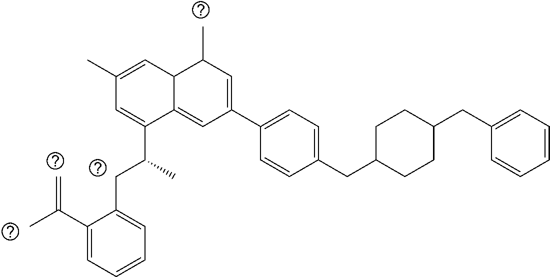 [SMILES: C=C(C)c1ccccc1C[C@@H](C)C1=CC(C)=CC2C1=CC(c1ccc(CC3CCC(Cc4ccccc4)CC3)cc1)=CC2C] | 400 |
| 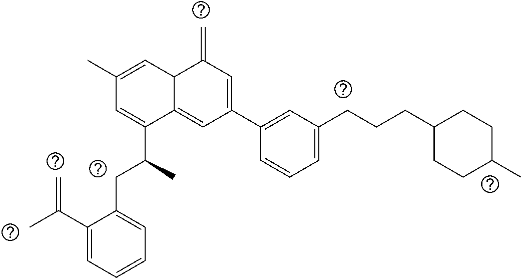 [SMILES: C=C(C)c1ccccc1C[C@H](C)C1=CC(C)=CC2C(=C)C=C(c3cccc(CCCC4CCC(C)CC4)c3)C=C12] | 401 |
| 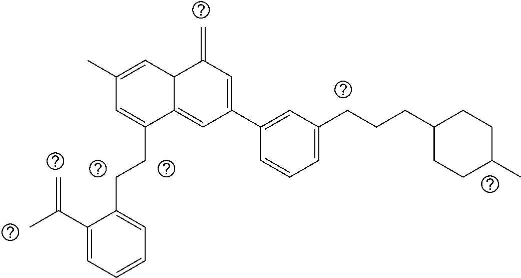 [SMILES: C=C(C)c1ccccc1CCC1=CC(C)=CC2C(=C)C=C(c3cccc(CCCC4CCC(C)CC4)c3)C=C12] | 402 |
| 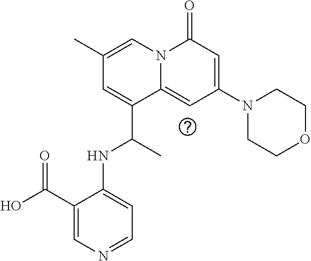 [SMILES: Cc1cc(C(C)Nc2ccncc2C(=O)O)c2cc(N3CCOCC3)cc(=O)n2c1] | 403 |
| 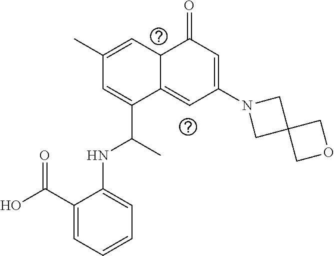 [SMILES: CC1=CC2C(=O)C=C(N3CC4(COC4)C3)C=C2C(C(C)Nc2ccccc2C(=O)O)=C1] | 404 |
| 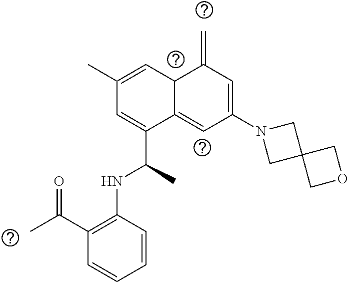 [SMILES: C=C1C=C(N2CC3(COC3)C2)C=C2C([C@@H](C)Nc3ccccc3C(C)=O)=CC(C)=CC12] | 405 |
| 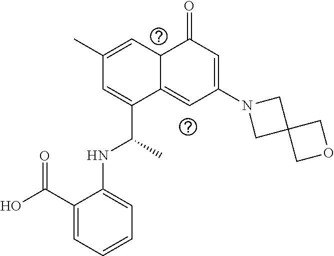 [SMILES: CC1=CC2C(=O)C=C(N3CC4(COC4)C3)C=C2C([C@H](C)Nc2ccccc2C(=O)O)=C1] | 406 |
| 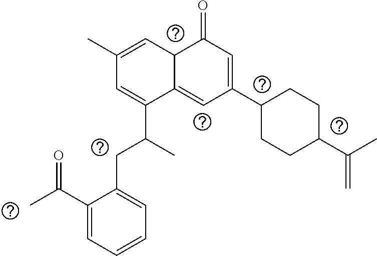 [SMILES: C=C(C)C1CCC(C2=CC(=O)C3C=C(C)C=C(C(C)Cc4ccccc4C(C)=O)C3=C2)CC1] | 407 |
| 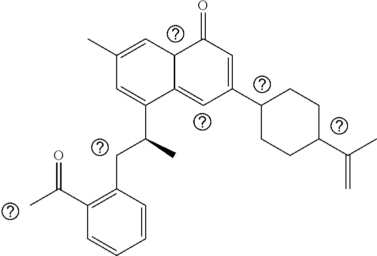 [SMILES: C=C(C)C1CCC(C2=CC(=O)C3C=C(C)C=C([C@@H](C)Cc4ccccc4C(C)=O)C3=C2)CC1] | 408 |
| 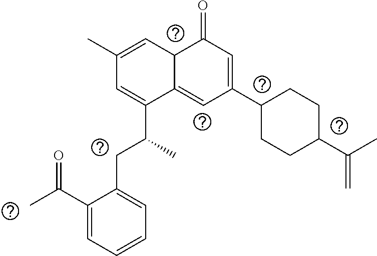 [SMILES: C=C(C)C1CCC(C2=CC(=O)C3C=C(C)C=C([C@H](C)Cc4ccccc4C(C)=O)C3=C2)CC1] | 409 |
| 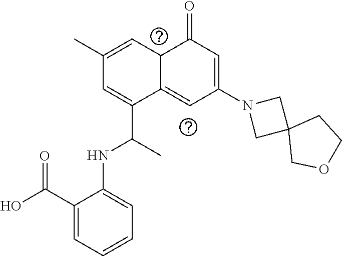 [SMILES: CC1=CC2C(=O)C=C(N3CC4(CCOC4)C3)C=C2C(C(C)Nc2ccccc2C(=O)O)=C1] | 410 |
| 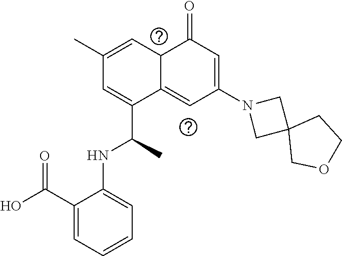 [SMILES: CC1=CC2C(=O)C=C(N3CC4(CCOC4)C3)C=C2C([C@@H](C)Nc2ccccc2C(=O)O)=C1] | 411 |
| 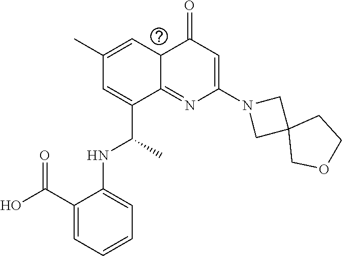 [SMILES: CC1=CC2C(=O)C=C(N3CC4(CCOC4)C3)N=C2C([C@H](C)Nc2ccccc2C(=O)O)=C1] | 412 |
| 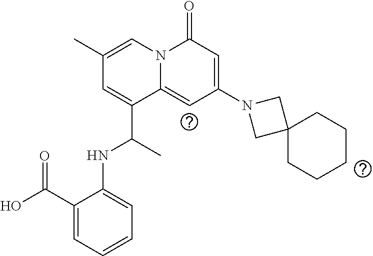 [SMILES: Cc1cc(C(C)Nc2ccccc2C(=O)O)c2cc(N3CC4(CCCCC4)C3)cc(=O)n2c1] | 413 |
| 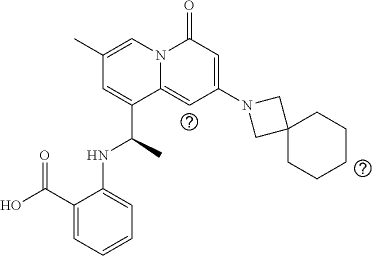 [SMILES: Cc1cc([C@@H](C)Nc2ccccc2C(=O)O)c2cc(N3CC4(CCCCC4)C3)cc(=O)n2c1] | 414 |
| 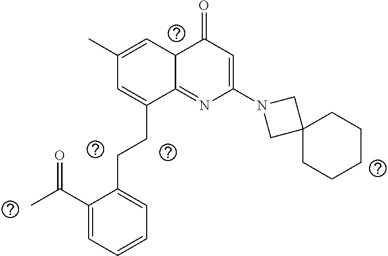 [SMILES: CC(=O)c1ccccc1CCC1=CC(C)=CC2C(=O)C=C(N3CC4(CCCCC4)C3)N=C12] | 415 |
| 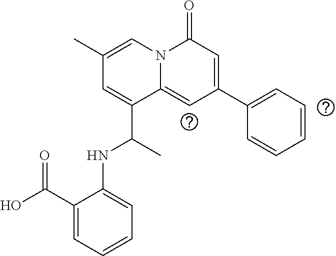 [SMILES: Cc1cc(C(C)Nc2ccccc2C(=O)O)c2cc(-c3ccccc3)cc(=O)n2c1] | 416 |
| 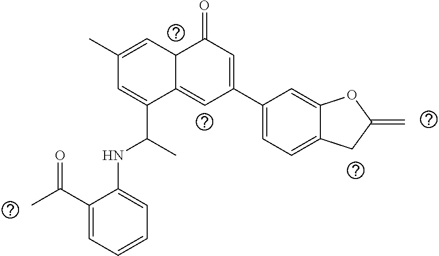 [SMILES: C=C1Cc2ccc(C3=CC(=O)C4C=C(C)C=C(C(C)Nc5ccccc5C(C)=O)C4=C3)cc2O1] | 417 |
| 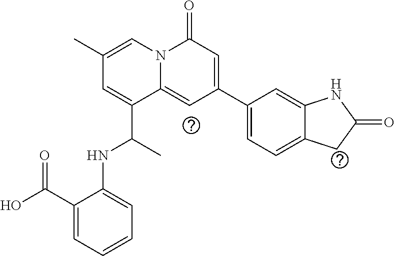 [SMILES: Cc1cc(C(C)Nc2ccccc2C(=O)O)c2cc(-c3ccc4c(c3)NC(=O)C4)cc(=O)n2c1] | 418 |
| 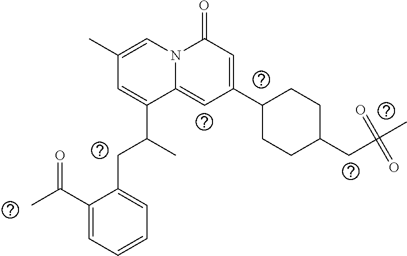 | 419 |
| 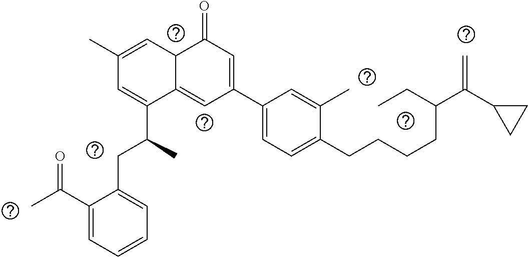 [SMILES: C=C(C(CC)CCCCc1ccc(C2=CC(=O)C3C=C(C)C=C([C@@H](C)Cc4ccccc4C(C)=O)C3=C2)cc1C)C1CC1] | 420 |
| 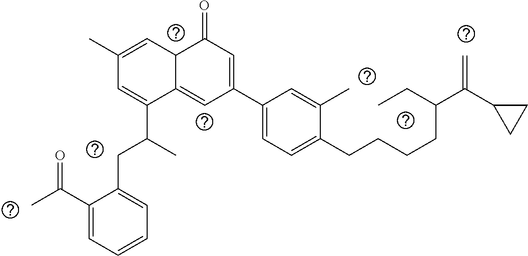 [SMILES: C=C(C(CC)CCCCc1ccc(C2=CC(=O)C3C=C(C)C=C(C(C)Cc4ccccc4C(C)=O)C3=C2)cc1C)C1CC1] | 421 |
| 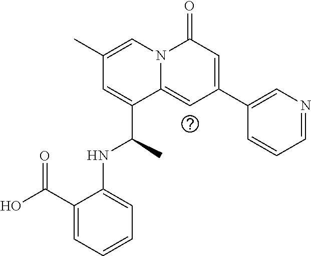 [SMILES: Cc1cc([C@@H](C)Nc2ccccc2C(=O)O)c2cc(-c3cccnc3)cc(=O)n2c1] | 422 |
| 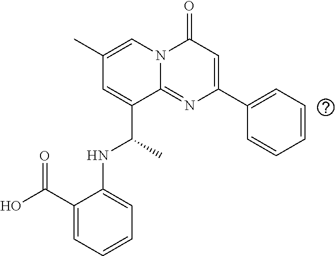 [SMILES: Cc1cc([C@H](C)Nc2ccccc2C(=O)O)c2nc(-c3ccccc3)cc(=O)n2c1] | 423 |
| 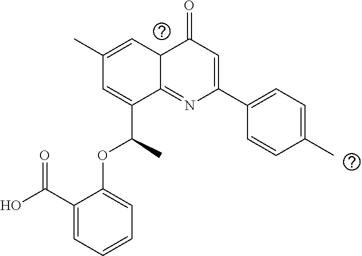 [SMILES: CC1=CC2C(=O)C=C(c3ccc(C)cc3)N=C2C([C@@H](C)Oc2ccccc2C(=O)O)=C1] | 424 |
| 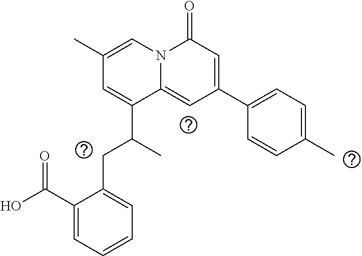 [SMILES: Cc1ccc(-c2cc(=O)n3cc(C)cc(C(C)Cc4ccccc4C(=O)O)c3c2)cc1] | 425 |
| 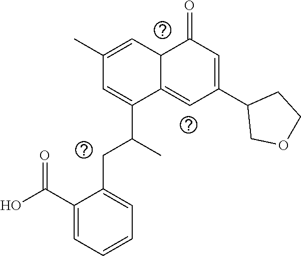 [SMILES: CC1=CC2C(=O)C=C(C3CCOC3)C=C2C(C(C)Cc2ccccc2C(=O)O)=C1] | 426 |
| 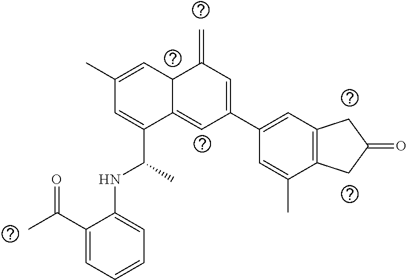 [SMILES: C=C1C=C(c2cc(C)c3c(c2)CC(=O)C3)C=C2C([C@H](C)Nc3ccccc3C(C)=O)=CC(C)=CC12] | 427 |
| 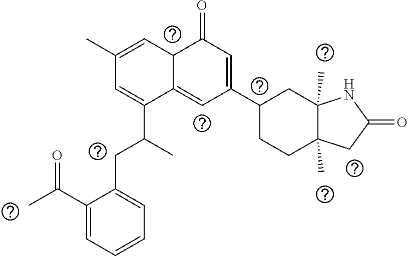 [SMILES: CC(=O)c1ccccc1CC(C)C1=CC(C)=CC2C(=O)C=C(C3CC[C@]4(C)CC(=O)N[C@]4(C)C3)C=C12] | 428 |
| 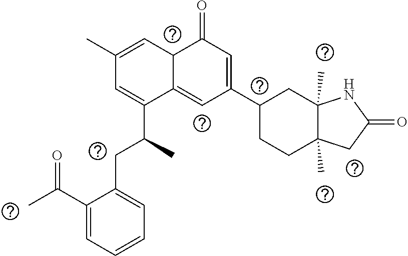 [SMILES: CC(=O)c1ccccc1C[C@H](C)C1=CC(C)=CC2C(=O)C=C(C3CC[C@]4(C)CC(=O)N[C@]4(C)C3)C=C12] | 429 |
| 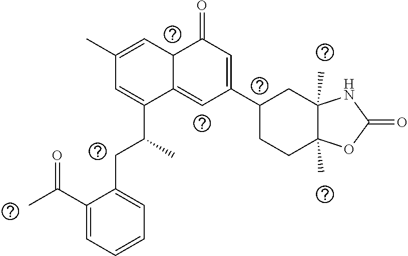 [SMILES: CC(=O)c1ccccc1C[C@@H](C)C1=CC(C)=CC2C(=O)C=C(C3CC[C@]4(C)OC(=O)N[C@]4(C)C3)C=C12] | 430 |
| 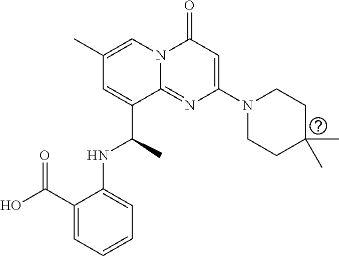 [SMILES: Cc1cc([C@@H](C)Nc2ccccc2C(=O)O)c2nc(N3CCC(C)(C)CC3)cc(=O)n2c1] | 431 |
| 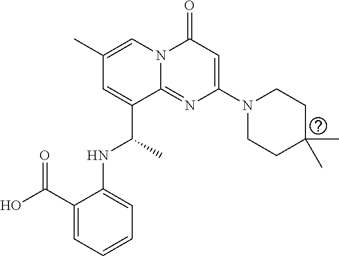 [SMILES: Cc1cc([C@H](C)Nc2ccccc2C(=O)O)c2nc(N3CCC(C)(C)CC3)cc(=O)n2c1] | 432 |
| 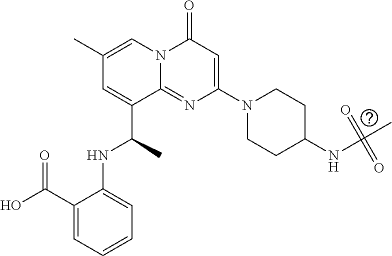 | 433 |
| 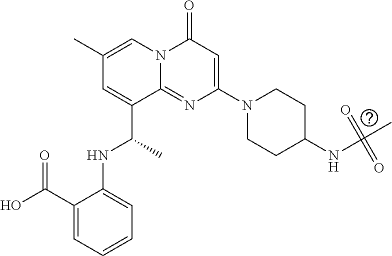 | 434 |
| 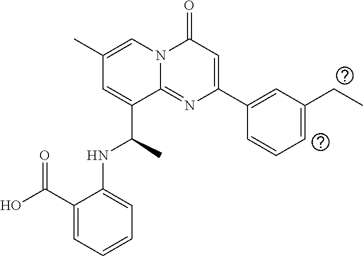 [SMILES: CCc1cccc(-c2cc(=O)n3cc(C)cc([C@@H](C)Nc4ccccc4C(=O)O)c3n2)c1] | 435 |
| 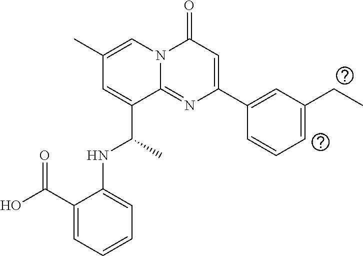 [SMILES: CCc1cccc(-c2cc(=O)n3cc(C)cc([C@H](C)Nc4ccccc4C(=O)O)c3n2)c1] | 436 |
| 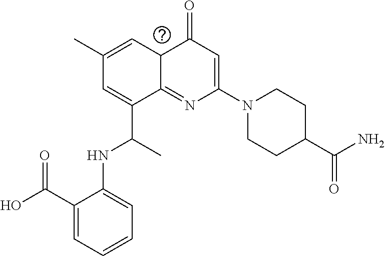 [SMILES: CC1=CC2C(=O)C=C(N3CCC(C(N)=O)CC3)N=C2C(C(C)Nc2ccccc2C(=O)O)=C1] | 437 |
| 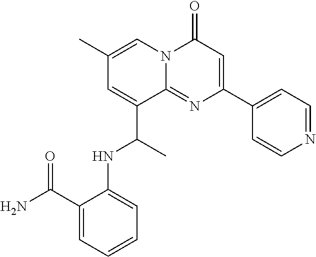 [SMILES: Cc1cc(C(C)Nc2ccccc2C(N)=O)c2nc(-c3ccncc3)cc(=O)n2c1] | 438 |
| 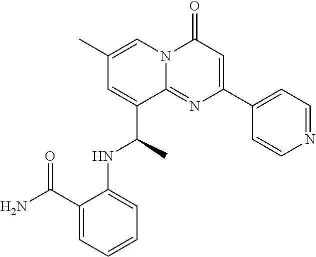 [SMILES: Cc1cc([C@@H](C)Nc2ccccc2C(N)=O)c2nc(-c3ccncc3)cc(=O)n2c1] | 439 |
| 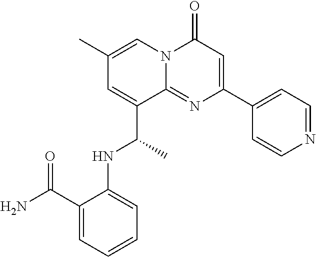 [SMILES: Cc1cc([C@H](C)Nc2ccccc2C(N)=O)c2nc(-c3ccncc3)cc(=O)n2c1] | 440 |
| 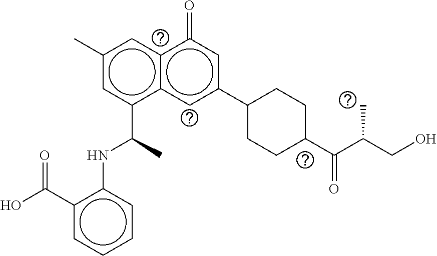 | 441 |
| 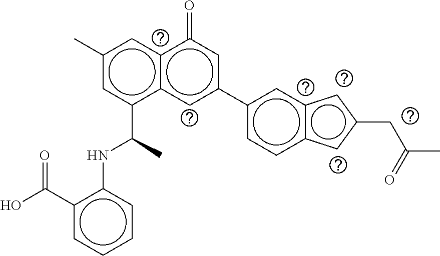 | 442 |
| 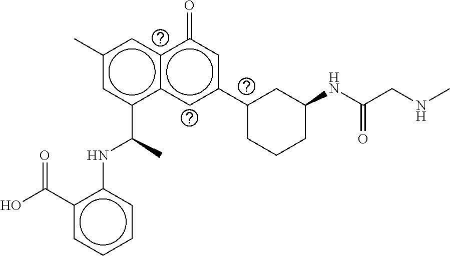 | 443 |
| 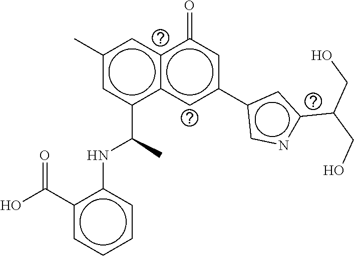 | 444 |
| 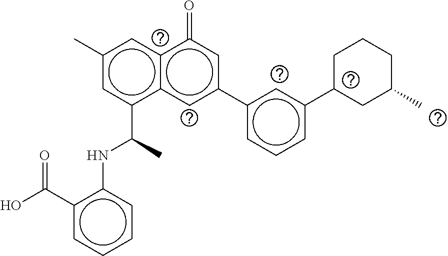 | 445 |
| 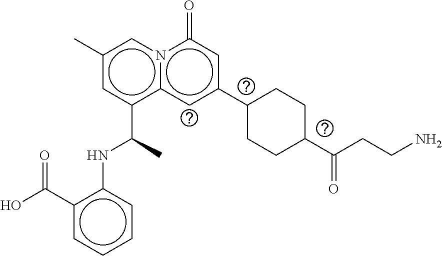 | 446 |
| 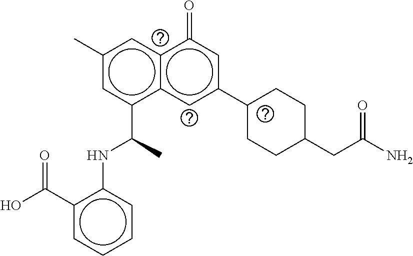 | 447 |
| 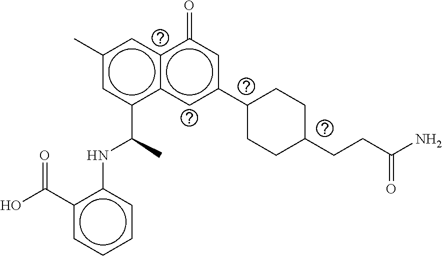 | 448 |
| 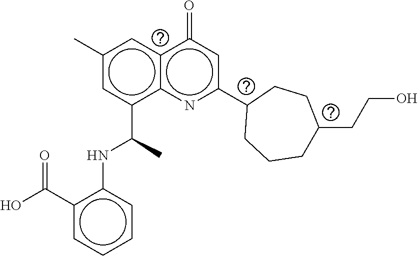 | 449 |
| 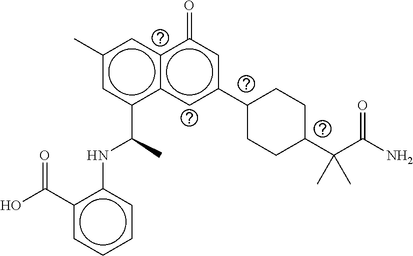 | 450 |
| 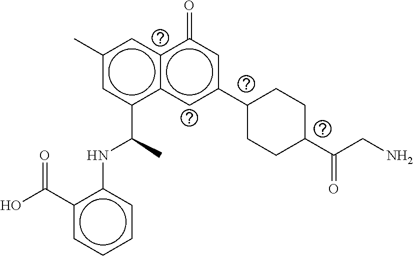 | 451 |
| 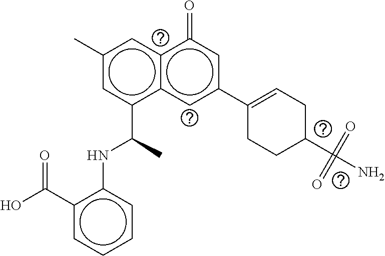 | 452 |
| 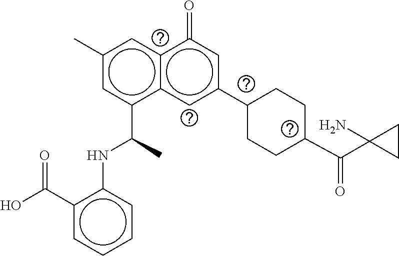 | 453 |
| 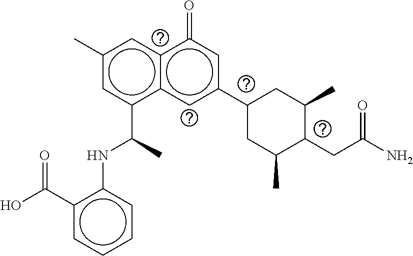 | 454 |
| 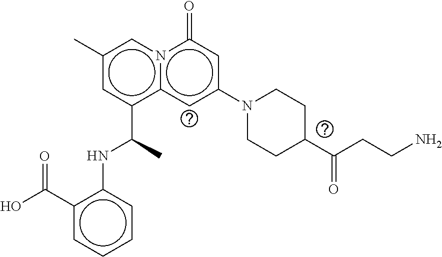 | 455 |
| 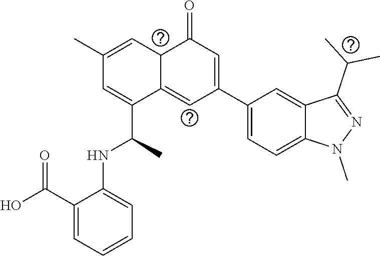 [SMILES: CC1=CC2C(=O)C=C(c3ccc4c(c3)c(C(C)C)nn4C)C=C2C([C@@H](C)Nc2ccccc2C(=O)O)=C1] | 456 |
| 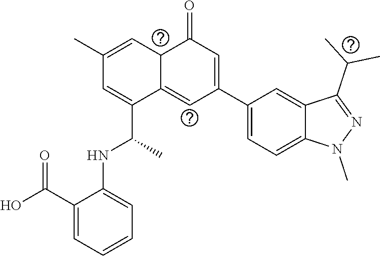 [SMILES: CC1=CC2C(=O)C=C(c3ccc4c(c3)c(C(C)C)nn4C)C=C2C([C@H](C)Nc2ccccc2C(=O)O)=C1] | 457 |
| 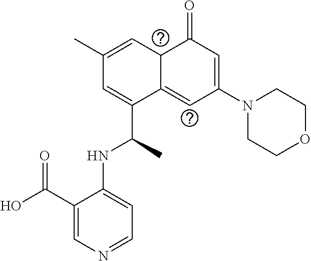 [SMILES: CC1=CC2C(=O)C=C(N3CCOCC3)C=C2C([C@@H](C)Nc2ccncc2C(=O)O)=C1] | 458 |
| 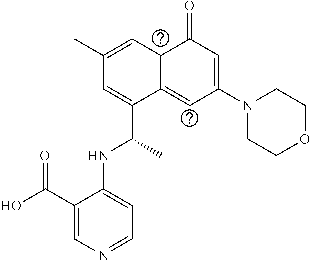 [SMILES: CC1=CC2C(=O)C=C(N3CCOCC3)C=C2C([C@H](C)Nc2ccncc2C(=O)O)=C1] | 459 |
| 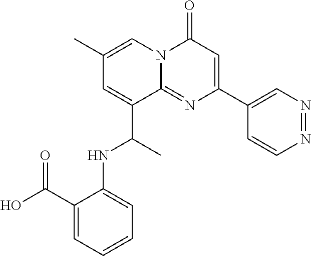 [SMILES: Cc1cc(C(C)Nc2ccccc2C(=O)O)c2nc(-c3ccnnc3)cc(=O)n2c1] | 460 |
| 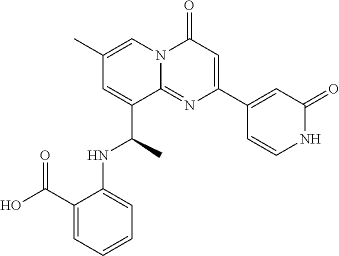 [SMILES: Cc1cc([C@@H](C)Nc2ccccc2C(=O)O)c2nc(-c3cc[nH]c(=O)c3)cc(=O)n2c1] | 461 |
| 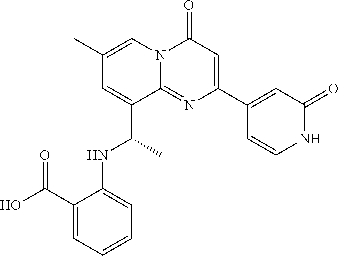 [SMILES: Cc1cc([C@H](C)Nc2ccccc2C(=O)O)c2nc(-c3cc[nH]c(=O)c3)cc(=O)n2c1] | 462 |
| 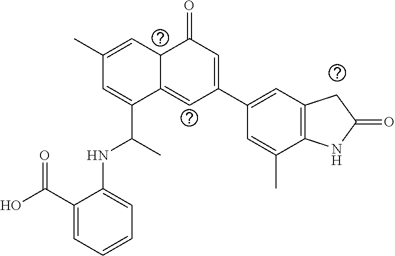 [SMILES: CC1=CC2C(=O)C=C(c3cc(C)c4c(c3)CC(=O)N4)C=C2C(C(C)Nc2ccccc2C(=O)O)=C1] | 463 |
| 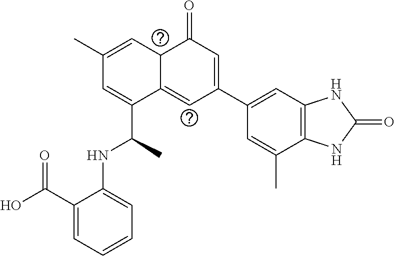 [SMILES: CC1=CC2C(=O)C=C(c3cc(C)c4[nH]c(=O)[nH]c4c3)C=C2C([C@@H](C)Nc2ccccc2C(=O)O)=C1] | 464 |
| 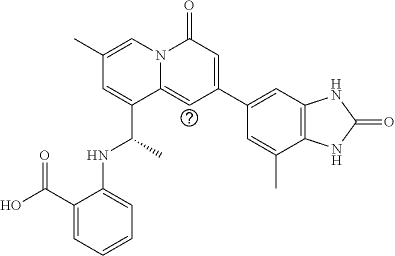 [SMILES: Cc1cc([C@H](C)Nc2ccccc2C(=O)O)c2cc(-c3cc(C)c4[nH]c(=O)[nH]c4c3)cc(=O)n2c1] | 465 |
| 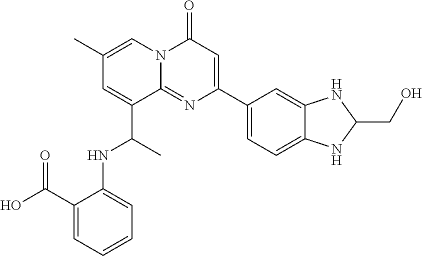 [SMILES: Cc1cc(C(C)Nc2ccccc2C(=O)O)c2nc(-c3ccc4c(c3)NC(CO)N4)cc(=O)n2c1] | 466 |
| 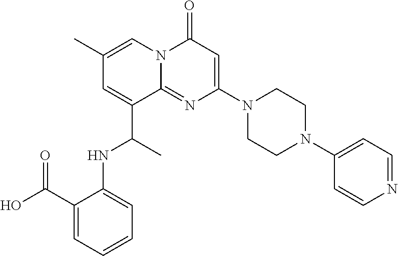 [SMILES: Cc1cc(C(C)Nc2ccccc2C(=O)O)c2nc(N3CCN(c4ccncc4)CC3)cc(=O)n2c1] | 467 |
| 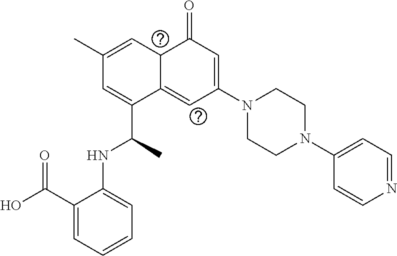 [SMILES: CC1=CC2C(=O)C=C(N3CCN(c4ccncc4)CC3)C=C2C([C@@H](C)Nc2ccccc2C(=O)O)=C1] | 468 |
| 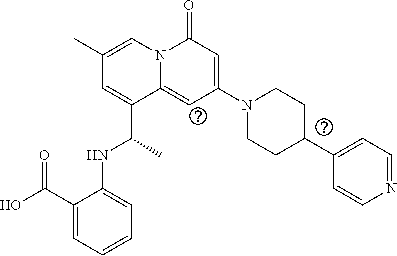 [SMILES: Cc1cc([C@H](C)Nc2ccccc2C(=O)O)c2cc(N3CCC(c4ccncc4)CC3)cc(=O)n2c1] | 469 |
| 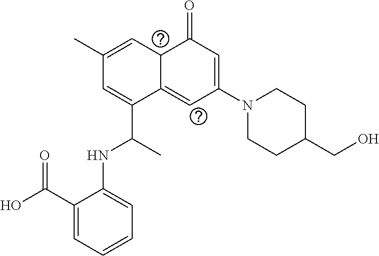 [SMILES: CC1=CC2C(=O)C=C(N3CCC(CO)CC3)C=C2C(C(C)Nc2ccccc2C(=O)O)=C1] | 470 |
| 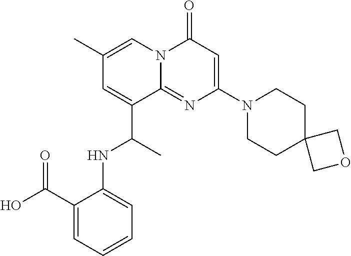 [SMILES: Cc1cc(C(C)Nc2ccccc2C(=O)O)c2nc(N3CCC4(CC3)COC4)cc(=O)n2c1] | 471 |
| 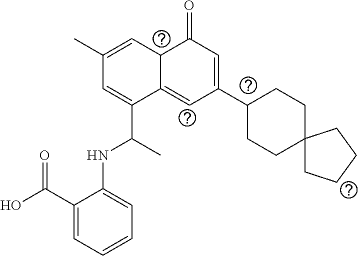 [SMILES: CC1=CC2C(=O)C=C(C3CCC4(CCCC4)CC3)C=C2C(C(C)Nc2ccccc2C(=O)O)=C1] | 472 |
| 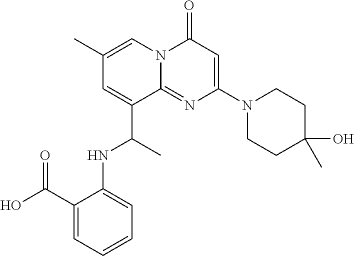 [SMILES: Cc1cc(C(C)Nc2ccccc2C(=O)O)c2nc(N3CCC(C)(O)CC3)cc(=O)n2c1] | 473 |
| 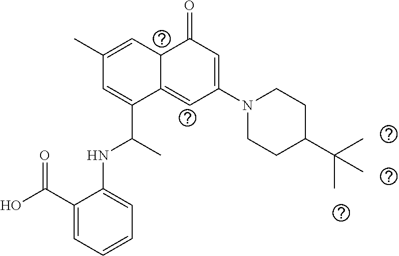 [SMILES: CC1=CC2C(=O)C=C(N3CCC(C(C)(C)C)CC3)C=C2C(C(C)Nc2ccccc2C(=O)O)=C1] | 474 |
| 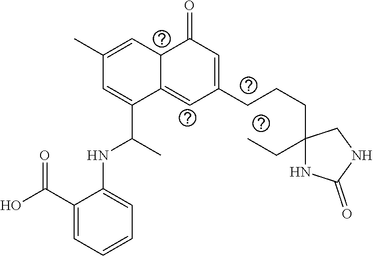 [SMILES: CCC1(CCCC2=CC(=O)C3C=C(C)C=C(C(C)Nc4ccccc4C(=O)O)C3=C2)CNC(=O)N1] | 475 |
| 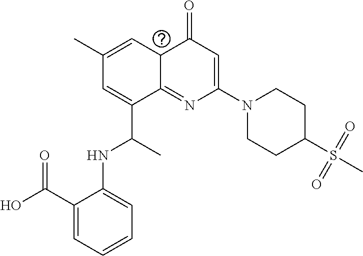 [SMILES: CC1=CC2C(=O)C=C(N3CCC(S(C)(=O)=O)CC3)N=C2C(C(C)Nc2ccccc2C(=O)O)=C1] | 476 |
| 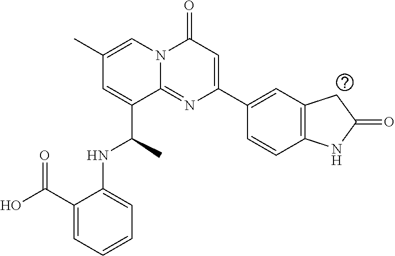 [SMILES: Cc1cc([C@@H](C)Nc2ccccc2C(=O)O)c2nc(-c3ccc4c(c3)CC(=O)N4)cc(=O)n2c1] | 477 |
| 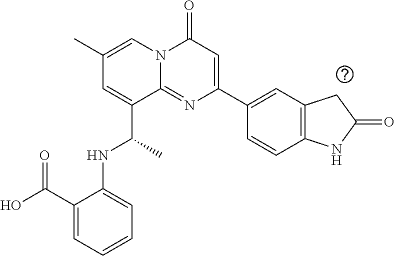 [SMILES: Cc1cc([C@H](C)Nc2ccccc2C(=O)O)c2nc(-c3ccc4c(c3)CC(=O)N4)cc(=O)n2c1] | 478 |
| 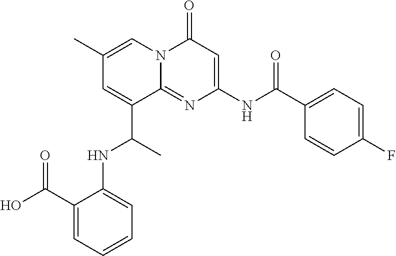 [SMILES: Cc1cc(C(C)Nc2ccccc2C(=O)O)c2nc(NC(=O)c3ccc(F)cc3)cc(=O)n2c1] | 479 |
| 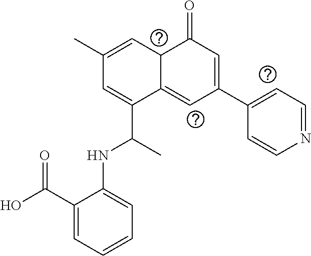 [SMILES: CC1=CC2C(=O)C=C(c3ccncc3)C=C2C(C(C)Nc2ccccc2C(=O)O)=C1] | 480 |
| 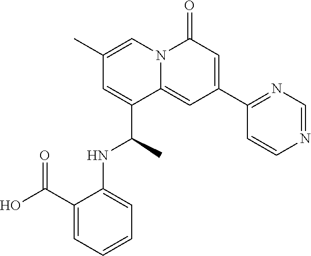 [SMILES: Cc1cc([C@@H](C)Nc2ccccc2C(=O)O)c2cc(-c3ccncn3)cc(=O)n2c1] | 481 |
| 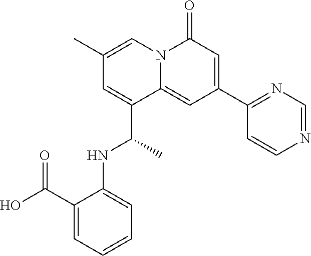 [SMILES: Cc1cc([C@H](C)Nc2ccccc2C(=O)O)c2cc(-c3ccncn3)cc(=O)n2c1] | 482 |
| 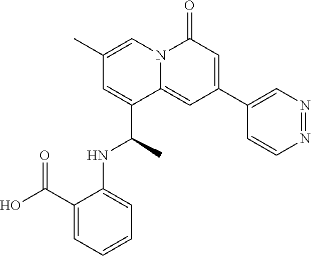 [SMILES: Cc1cc([C@@H](C)Nc2ccccc2C(=O)O)c2cc(-c3ccnnc3)cc(=O)n2c1] | 483 |
| 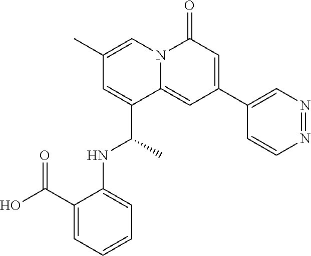 [SMILES: Cc1cc([C@H](C)Nc2ccccc2C(=O)O)c2cc(-c3ccnnc3)cc(=O)n2c1] | 484 |
| 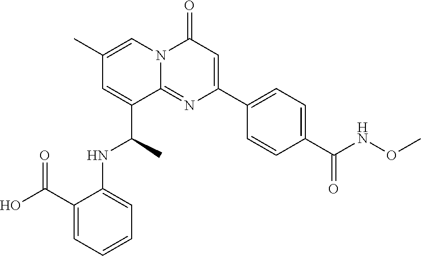 [SMILES: CONC(=O)c1ccc(-c2cc(=O)n3cc(C)cc([C@@H](C)Nc4ccccc4C(=O)O)c3n2)cc1] | 485 |
| 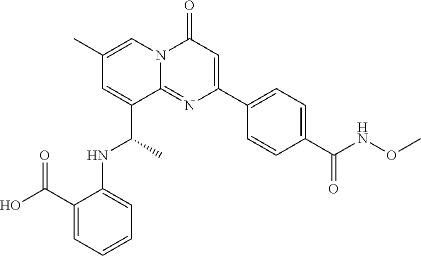 [SMILES: CONC(=O)c1ccc(-c2cc(=O)n3cc(C)cc([C@H](C)Nc4ccccc4C(=O)O)c3n2)cc1] | 486 |
| 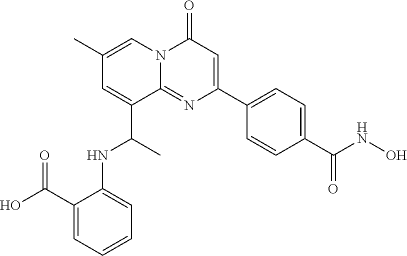 [SMILES: Cc1cc(C(C)Nc2ccccc2C(=O)O)c2nc(-c3ccc(C(=O)NO)cc3)cc(=O)n2c1] | 487 |
| 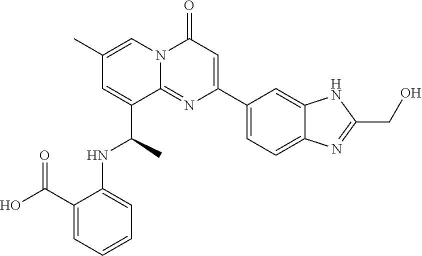 [SMILES: Cc1cc([C@@H](C)Nc2ccccc2C(=O)O)c2nc(-c3ccc4nc(CO)[nH]c4c3)cc(=O)n2c1] | 488 |
| 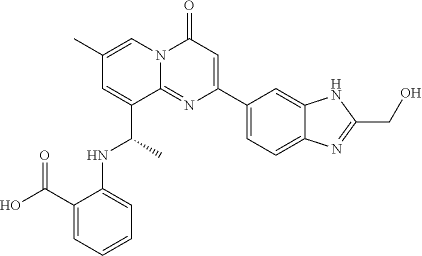 [SMILES: Cc1cc([C@H](C)Nc2ccccc2C(=O)O)c2nc(-c3ccc4nc(CO)[nH]c4c3)cc(=O)n2c1] | 489 |
| 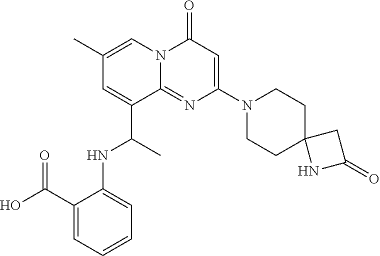 [SMILES: Cc1cc(C(C)Nc2ccccc2C(=O)O)c2nc(N3CCC4(CC3)CC(=O)N4)cc(=O)n2c1] | 490 |
| 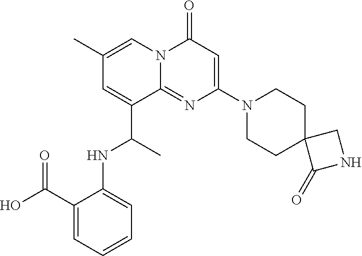 [SMILES: Cc1cc(C(C)Nc2ccccc2C(=O)O)c2nc(N3CCC4(CC3)CNC4=O)cc(=O)n2c1] | 491 |
| 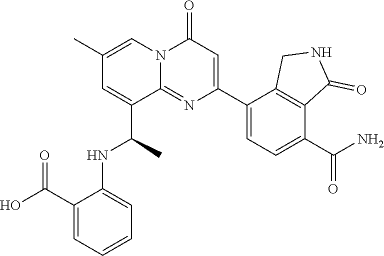 [SMILES: Cc1cc([C@@H](C)Nc2ccccc2C(=O)O)c2nc(-c3ccc(C(N)=O)c4c3CNC4=O)cc(=O)n2c1] | 492 |
| 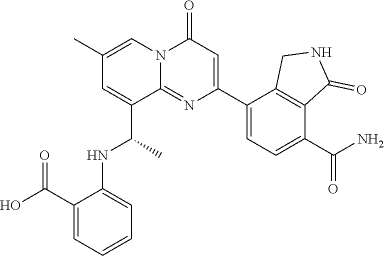 [SMILES: Cc1cc([C@H](C)Nc2ccccc2C(=O)O)c2nc(-c3ccc(C(N)=O)c4c3CNC4=O)cc(=O)n2c1] | 493 |
| 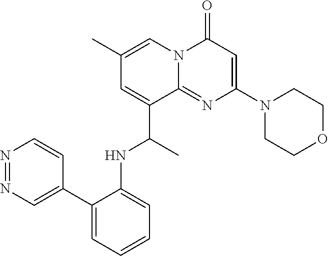 [SMILES: Cc1cc(C(C)Nc2ccccc2-c2ccnnc2)c2nc(N3CCOCC3)cc(=O)n2c1] | 494 |
| 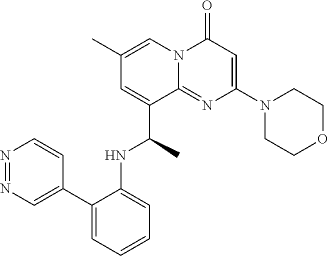 [SMILES: Cc1cc([C@@H](C)Nc2ccccc2-c2ccnnc2)c2nc(N3CCOCC3)cc(=O)n2c1] | 495 |
| 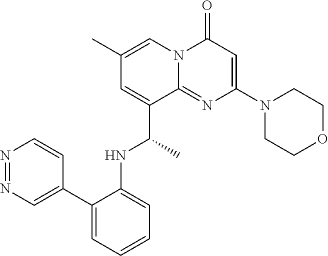 [SMILES: Cc1cc([C@H](C)Nc2ccccc2-c2ccnnc2)c2nc(N3CCOCC3)cc(=O)n2c1] | 496 |
| 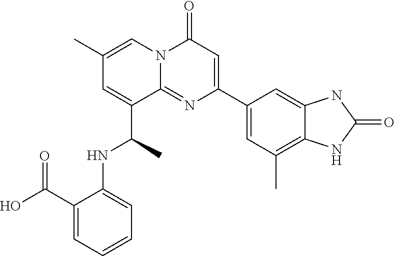 [SMILES: Cc1cc([C@@H](C)Nc2ccccc2C(=O)O)c2nc(-c3cc(C)c4[nH]c(=O)[nH]c4c3)cc(=O)n2c1] | 497 |
| 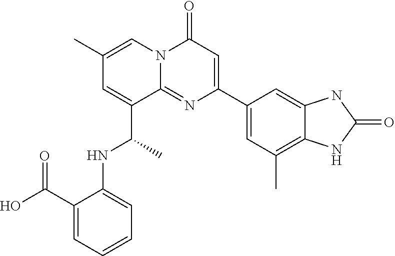 [SMILES: Cc1cc([C@H](C)Nc2ccccc2C(=O)O)c2nc(-c3cc(C)c4[nH]c(=O)[nH]c4c3)cc(=O)n2c1] | 498 |
| 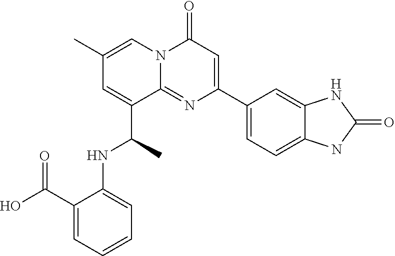 [SMILES: Cc1cc([C@@H](C)Nc2ccccc2C(=O)O)c2nc(-c3ccc4[nH]c(=O)[nH]c4c3)cc(=O)n2c1] | 499 |
| 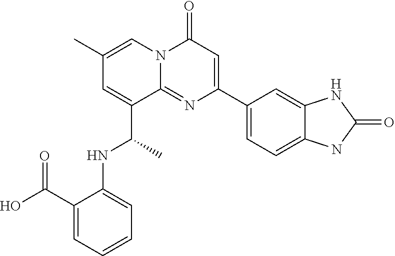 [SMILES: Cc1cc([C@H](C)Nc2ccccc2C(=O)O)c2nc(-c3ccc4[nH]c(=O)[nH]c4c3)cc(=O)n2c1] | 500 |
| 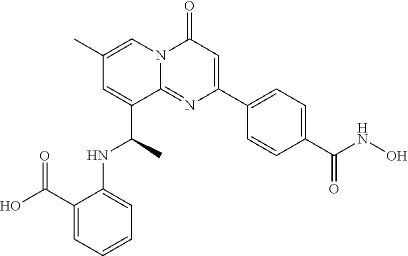 [SMILES: Cc1cc([C@@H](C)Nc2ccccc2C(=O)O)c2nc(-c3ccc(C(=O)NO)cc3)cc(=O)n2c1] | 501 |
| 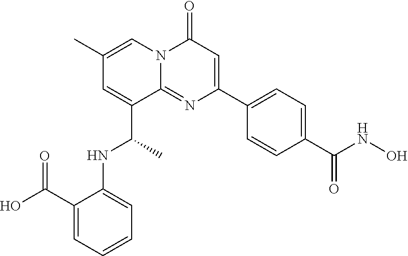 [SMILES: Cc1cc([C@H](C)Nc2ccccc2C(=O)O)c2nc(-c3ccc(C(=O)NO)cc3)cc(=O)n2c1] | 502 |
| 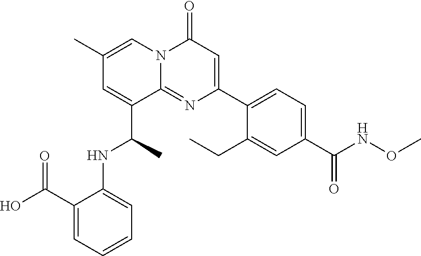 [SMILES: CCc1cc(C(=O)NOC)ccc1-c1cc(=O)n2cc(C)cc([C@@H](C)Nc3ccccc3C(=O)O)c2n1] | 503 |
| 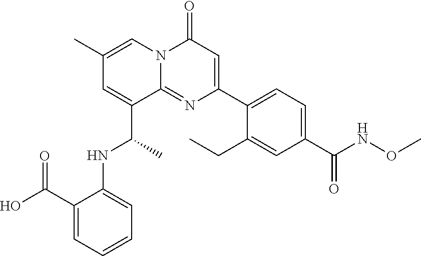 [SMILES: CCc1cc(C(=O)NOC)ccc1-c1cc(=O)n2cc(C)cc([C@H](C)Nc3ccccc3C(=O)O)c2n1] | 504 |
| 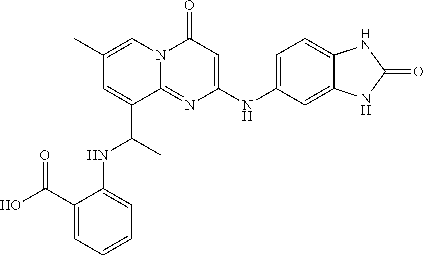 [SMILES: Cc1cc(C(C)Nc2ccccc2C(=O)O)c2nc(Nc3ccc4[nH]c(=O)[nH]c4c3)cc(=O)n2c1] | 505 |
| 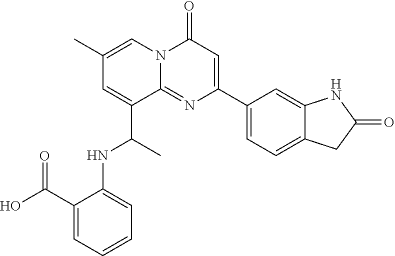 [SMILES: Cc1cc(C(C)Nc2ccccc2C(=O)O)c2nc(-c3ccc4c(c3)NC(=O)C4)cc(=O)n2c1] | 506 |
| 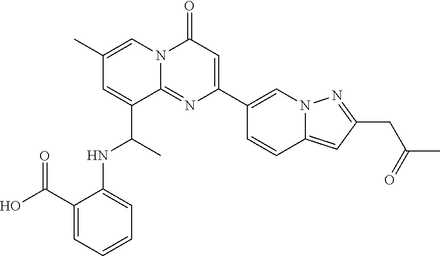 [SMILES: CC(=O)Cc1cc2ccc(-c3cc(=O)n4cc(C)cc(C(C)Nc5ccccc5C(=O)O)c4n3)cn2n1] | 507 |
| 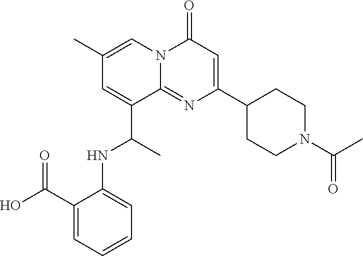 [SMILES: CC(=O)N1CCC(c2cc(=O)n3cc(C)cc(C(C)Nc4ccccc4C(=O)O)c3n2)CC1] | 508 |
| 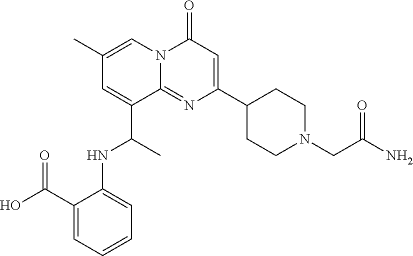 [SMILES: Cc1cc(C(C)Nc2ccccc2C(=O)O)c2nc(C3CCN(CC(N)=O)CC3)cc(=O)n2c1] | 509 |
| 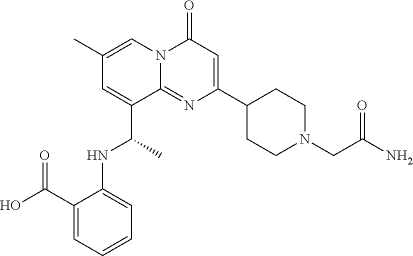 [SMILES: Cc1cc([C@H](C)Nc2ccccc2C(=O)O)c2nc(C3CCN(CC(N)=O)CC3)cc(=O)n2c1] | 510 |
| 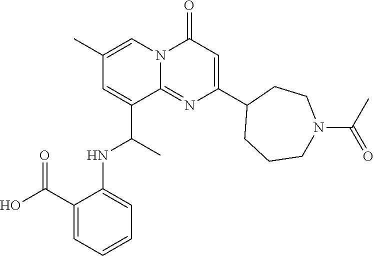 [SMILES: CC(=O)N1CCCC(c2cc(=O)n3cc(C)cc(C(C)Nc4ccccc4C(=O)O)c3n2)CC1] | 511 |
| 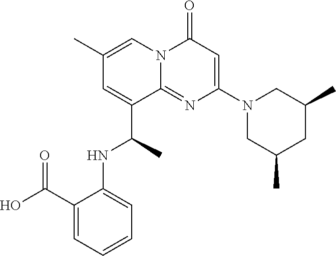 [SMILES: Cc1cc([C@@H](C)Nc2ccccc2C(=O)O)c2nc(N3C[C@H](C)C[C@H](C)C3)cc(=O)n2c1] | 512 |
| 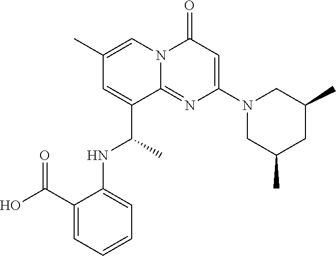 [SMILES: Cc1cc([C@H](C)Nc2ccccc2C(=O)O)c2nc(N3C[C@H](C)C[C@H](C)C3)cc(=O)n2c1] | 513 |
| 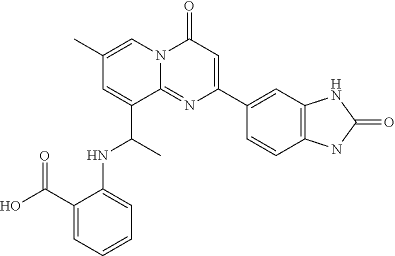 [SMILES: Cc1cc(C(C)Nc2ccccc2C(=O)O)c2nc(-c3ccc4[nH]c(=O)[nH]c4c3)cc(=O)n2c1] | 514 |
| 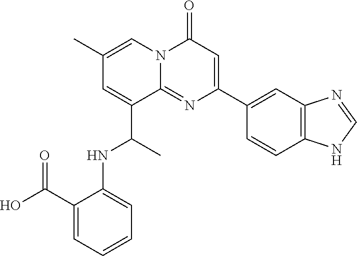 [SMILES: Cc1cc(C(C)Nc2ccccc2C(=O)O)c2nc(-c3ccc4[nH]cnc4c3)cc(=O)n2c1] | 515 |
| 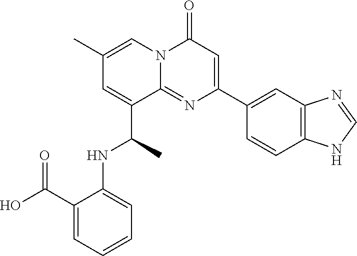 [SMILES: Cc1cc([C@@H](C)Nc2ccccc2C(=O)O)c2nc(-c3ccc4[nH]cnc4c3)cc(=O)n2c1] | 516 |
| 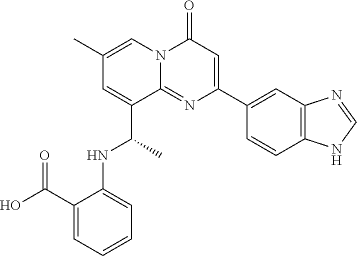 [SMILES: Cc1cc([C@H](C)Nc2ccccc2C(=O)O)c2nc(-c3ccc4[nH]cnc4c3)cc(=O)n2c1] | 517 |
| 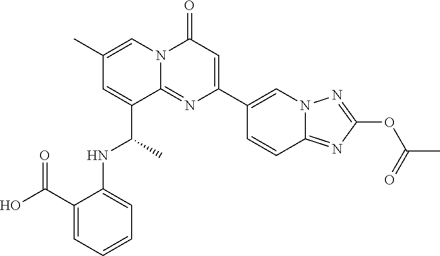 [SMILES: CC(=O)Oc1nc2ccc(-c3cc(=O)n4cc(C)cc([C@H](C)Nc5ccccc5C(=O)O)c4n3)cn2n1] | 518 |
| 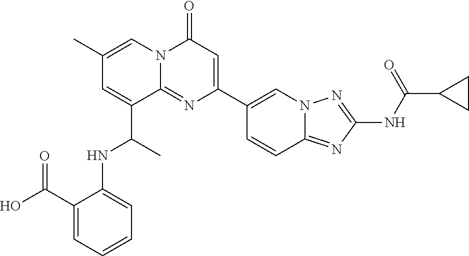 [SMILES: Cc1cc(C(C)Nc2ccccc2C(=O)O)c2nc(-c3ccc4nc(NC(=O)C5CC5)nn4c3)cc(=O)n2c1] | 519 |
| 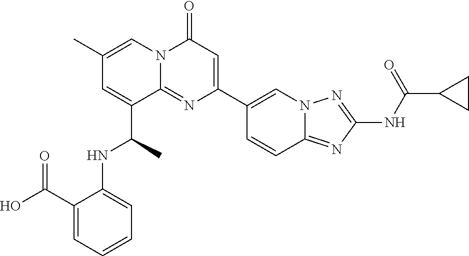 [SMILES: Cc1cc([C@@H](C)Nc2ccccc2C(=O)O)c2nc(-c3ccc4nc(NC(=O)C5CC5)nn4c3)cc(=O)n2c1] | 520 |
| 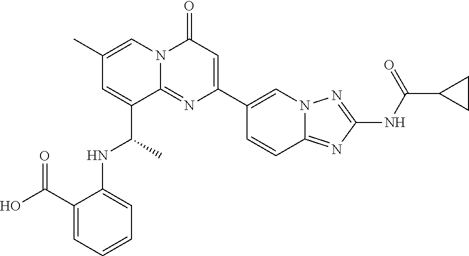 [SMILES: Cc1cc([C@H](C)Nc2ccccc2C(=O)O)c2nc(-c3ccc4nc(NC(=O)C5CC5)nn4c3)cc(=O)n2c1] | 521 |
| 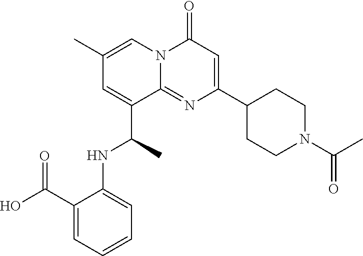 [SMILES: CC(=O)N1CCC(c2cc(=O)n3cc(C)cc([C@@H](C)Nc4ccccc4C(=O)O)c3n2)CC1] | 522 |
| 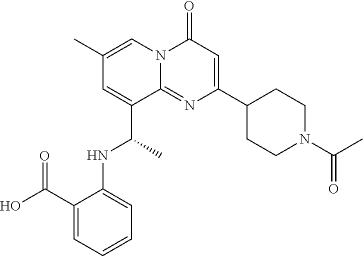 [SMILES: CC(=O)N1CCC(c2cc(=O)n3cc(C)cc([C@H](C)Nc4ccccc4C(=O)O)c3n2)CC1] | 523 |
| 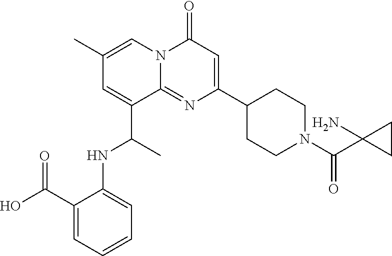 [SMILES: Cc1cc(C(C)Nc2ccccc2C(=O)O)c2nc(C3CCN(C(=O)C4(N)CC4)CC3)cc(=O)n2c1] | 524 |
| 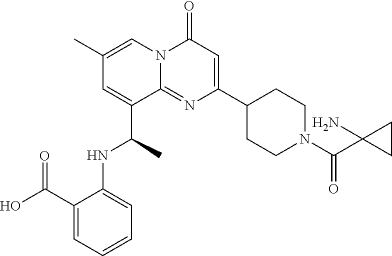 [SMILES: Cc1cc([C@@H](C)Nc2ccccc2C(=O)O)c2nc(C3CCN(C(=O)C4(N)CC4)CC3)cc(=O)n2c1] | 525 |
| 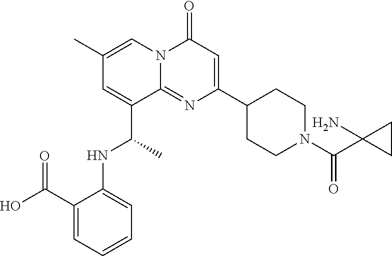 [SMILES: Cc1cc([C@H](C)Nc2ccccc2C(=O)O)c2nc(C3CCN(C(=O)C4(N)CC4)CC3)cc(=O)n2c1] | 526 |
| 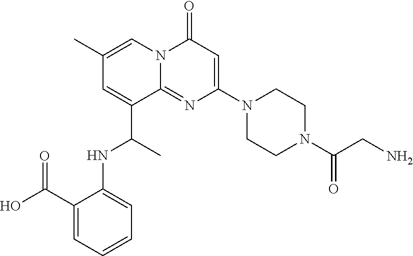 [SMILES: Cc1cc(C(C)Nc2ccccc2C(=O)O)c2nc(N3CCN(C(=O)CN)CC3)cc(=O)n2c1] | 527 |
| 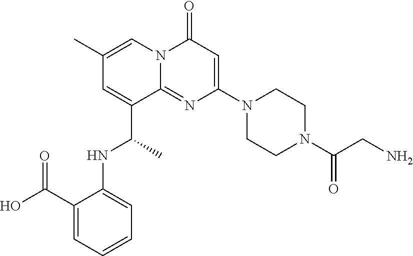 [SMILES: Cc1cc([C@H](C)Nc2ccccc2C(=O)O)c2nc(N3CCN(C(=O)CN)CC3)cc(=O)n2c1] | 528 |
| 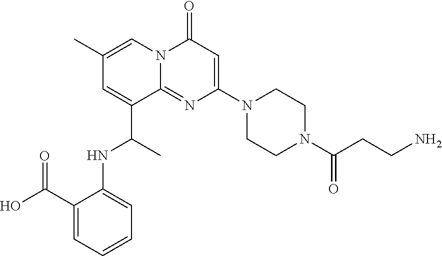 [SMILES: Cc1cc(C(C)Nc2ccccc2C(=O)O)c2nc(N3CCN(C(=O)CCN)CC3)cc(=O)n2c1] | 529 |
| 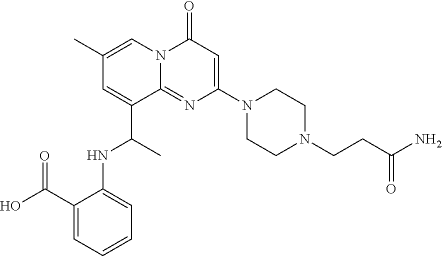 [SMILES: Cc1cc(C(C)Nc2ccccc2C(=O)O)c2nc(N3CCN(CCC(N)=O)CC3)cc(=O)n2c1] | 530 |
| 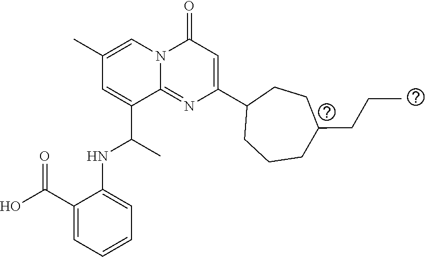 [SMILES: CCCC1CCCC(c2cc(=O)n3cc(C)cc(C(C)Nc4ccccc4C(=O)O)c3n2)CC1] | 531 |
| 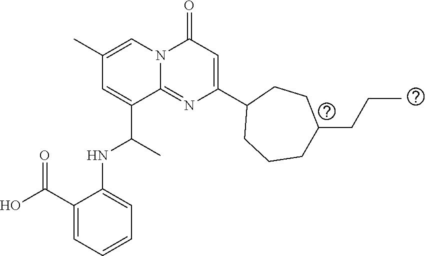 [SMILES: CCCC1CCCC(c2cc(=O)n3cc(C)cc(C(C)Nc4ccccc4C(=O)O)c3n2)CC1] | 532 |
| 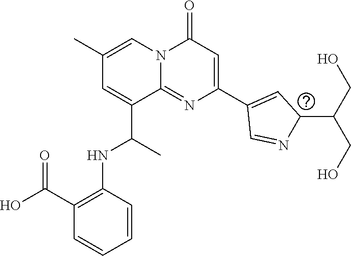 [SMILES: Cc1cc(C(C)Nc2ccccc2C(=O)O)c2nc(C3=CC(C(CO)CO)N=C3)cc(=O)n2c1] | 533 |
| 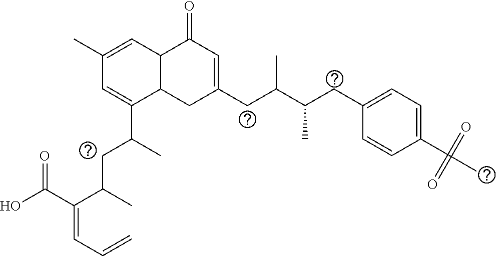 | 534 |
| 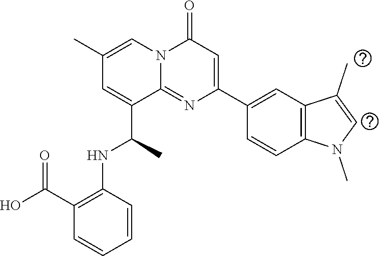 [SMILES: Cc1cc([C@@H](C)Nc2ccccc2C(=O)O)c2nc(-c3ccc4c(c3)c(C)cn4C)cc(=O)n2c1] | 535 |
| 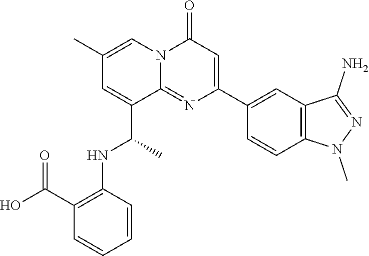 [SMILES: Cc1cc([C@H](C)Nc2ccccc2C(=O)O)c2nc(-c3ccc4c(c3)c(N)nn4C)cc(=O)n2c1] | 536 |
| 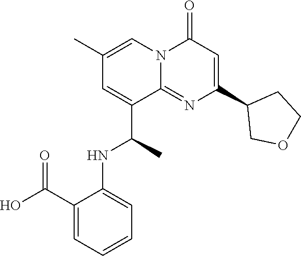 [SMILES: Cc1cc([C@@H](C)Nc2ccccc2C(=O)O)c2nc([C@H]3CCOC3)cc(=O)n2c1] | 537 |
| 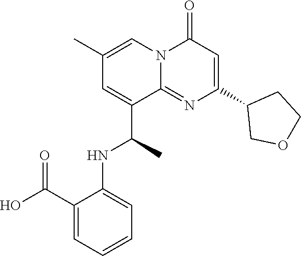 [SMILES: Cc1cc([C@@H](C)Nc2ccccc2C(=O)O)c2nc([C@@H]3CCOC3)cc(=O)n2c1] | 538 |
| 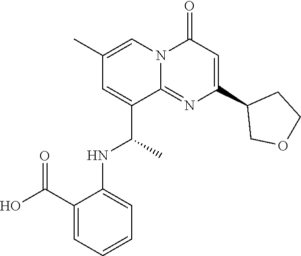 [SMILES: Cc1cc([C@H](C)Nc2ccccc2C(=O)O)c2nc([C@H]3CCOC3)cc(=O)n2c1] | 539 |
| 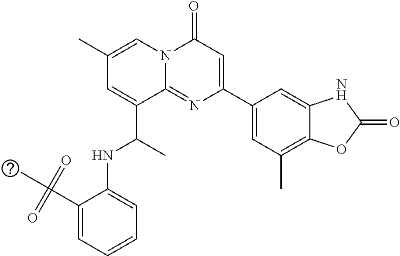 | 540 |
| 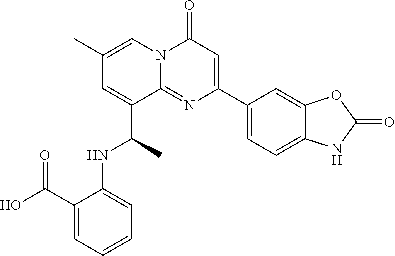 [SMILES: Cc1cc([C@@H](C)Nc2ccccc2C(=O)O)c2nc(-c3ccc4[nH]c(=O)oc4c3)cc(=O)n2c1] | 541 |
| 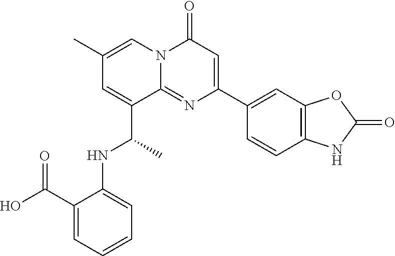 [SMILES: Cc1cc([C@H](C)Nc2ccccc2C(=O)O)c2nc(-c3ccc4[nH]c(=O)oc4c3)cc(=O)n2c1] | 542 |
| 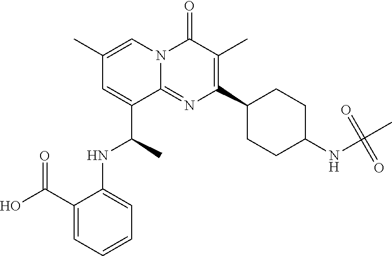 | 543 |
| 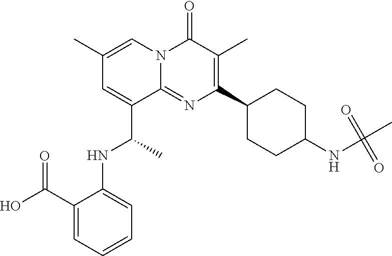 | 544 |
| 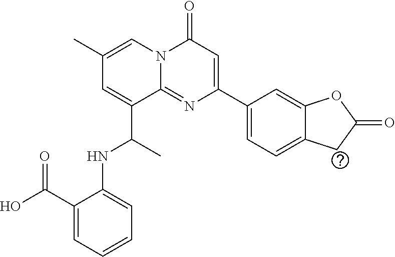 [SMILES: Cc1cc(C(C)Nc2ccccc2C(=O)O)c2nc(-c3ccc4c(c3)OC(=O)C4)cc(=O)n2c1] | 545 |
| 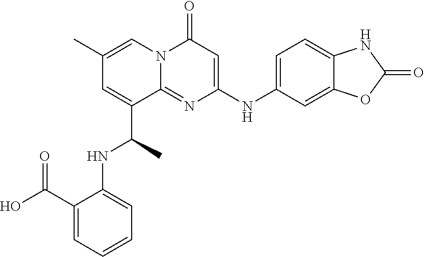 [SMILES: Cc1cc([C@@H](C)Nc2ccccc2C(=O)O)c2nc(Nc3ccc4[nH]c(=O)oc4c3)cc(=O)n2c1] | 546 |
| 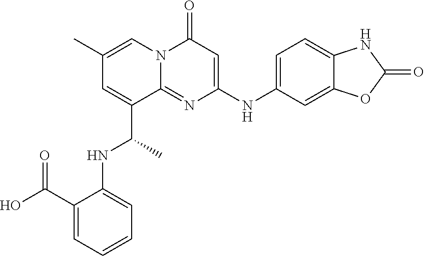 [SMILES: Cc1cc([C@H](C)Nc2ccccc2C(=O)O)c2nc(Nc3ccc4[nH]c(=O)oc4c3)cc(=O)n2c1] | 547 |
| 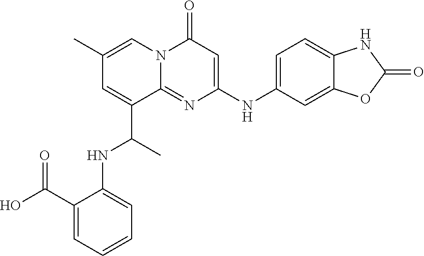 [SMILES: Cc1cc(C(C)Nc2ccccc2C(=O)O)c2nc(Nc3ccc4[nH]c(=O)oc4c3)cc(=O)n2c1] | 548 |
| 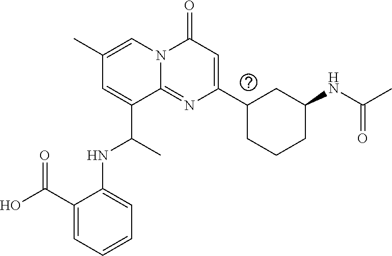 [SMILES: CC(=O)N[C@H]1CCCC(c2cc(=O)n3cc(C)cc(C(C)Nc4ccccc4C(=O)O)c3n2)C1] | 549 |
| 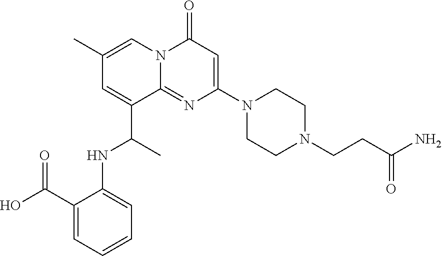 [SMILES: Cc1cc(C(C)Nc2ccccc2C(=O)O)c2nc(N3CCN(CCC(N)=O)CC3)cc(=O)n2c1] | 550 |
| 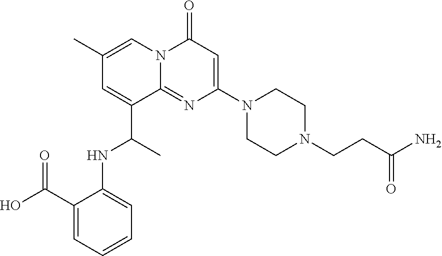 [SMILES: Cc1cc(C(C)Nc2ccccc2C(=O)O)c2nc(N3CCN(CCC(N)=O)CC3)cc(=O)n2c1] | 551 |
| 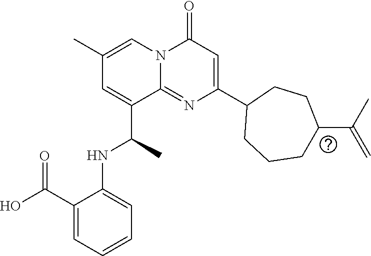 [SMILES: C=C(C)C1CCCC(c2cc(=O)n3cc(C)cc([C@@H](C)Nc4ccccc4C(=O)O)c3n2)CC1] | 552 |
| 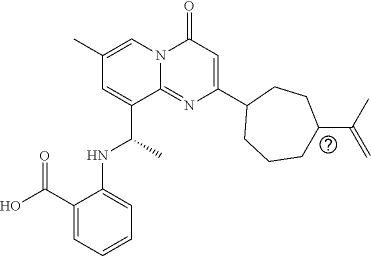 [SMILES: C=C(C)C1CCCC(c2cc(=O)n3cc(C)cc([C@H](C)Nc4ccccc4C(=O)O)c3n2)CC1] | 553 |
| 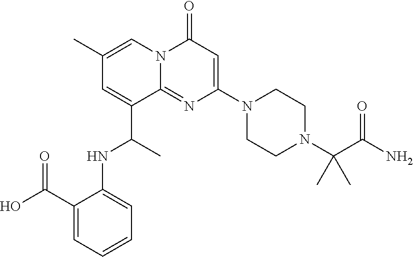 [SMILES: Cc1cc(C(C)Nc2ccccc2C(=O)O)c2nc(N3CCN(C(C)(C)C(N)=O)CC3)cc(=O)n2c1] | 554 |
| 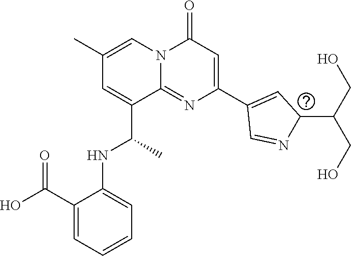 [SMILES: Cc1cc([C@H](C)Nc2ccccc2C(=O)O)c2nc(C3=CC(C(CO)CO)N=C3)cc(=O)n2c1] | 555 |
| 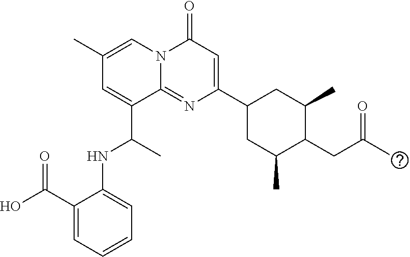 [SMILES: CC(=O)CC1[C@H](C)CC(c2cc(=O)n3cc(C)cc(C(C)Nc4ccccc4C(=O)O)c3n2)C[C@@H]1C] | 556 |
| 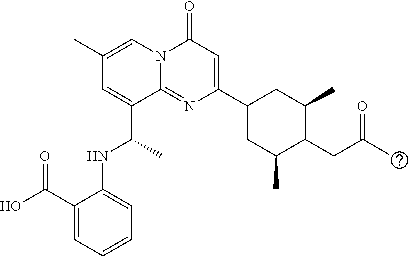 [SMILES: CC(=O)CC1[C@H](C)CC(c2cc(=O)n3cc(C)cc([C@H](C)Nc4ccccc4C(=O)O)c3n2)C[C@@H]1C] | 557 |
| 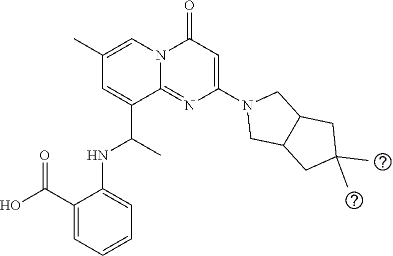 [SMILES: Cc1cc(C(C)Nc2ccccc2C(=O)O)c2nc(N3CC4CC(C)(C)CC4C3)cc(=O)n2c1] | 558 |
| 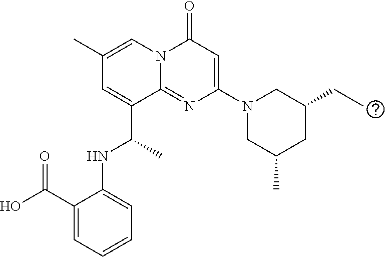 [SMILES: CC[C@@H]1C[C@H](C)CN(c2cc(=O)n3cc(C)cc([C@H](C)Nc4ccccc4C(=O)O)c3n2)C1] | 559 |
| 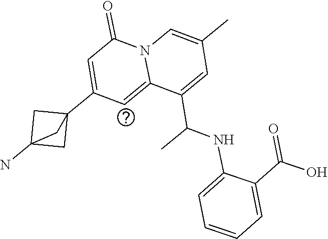 [SMILES: Cc1cc(C(C)Nc2ccccc2C(=O)O)c2cc(C34CC(N)(C3)C4)cc(=O)n2c1] | 560 |
| 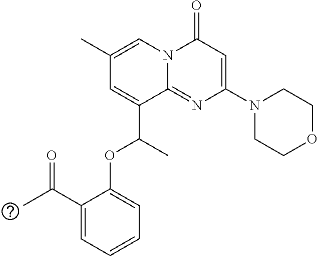 [SMILES: CC(=O)c1ccccc1OC(C)c1cc(C)cn2c(=O)cc(N3CCOCC3)nc12] | 561 |
| 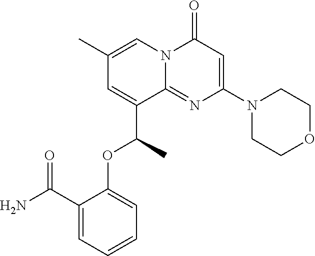 [SMILES: Cc1cc([C@@H](C)Oc2ccccc2C(N)=O)c2nc(N3CCOCC3)cc(=O)n2c1] | 562 |
| 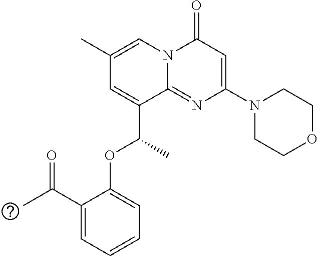 [SMILES: CC(=O)c1ccccc1O[C@@H](C)c1cc(C)cn2c(=O)cc(N3CCOCC3)nc12] | 563 |
| 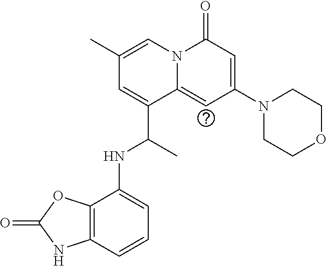 [SMILES: Cc1cc(C(C)Nc2cccc3[nH]c(=O)oc23)c2cc(N3CCOCC3)cc(=O)n2c1] | 564 |
| 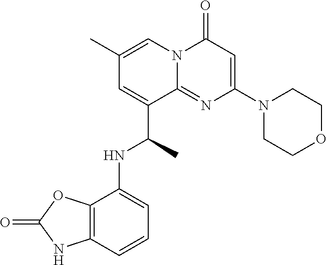 [SMILES: Cc1cc([C@@H](C)Nc2cccc3[nH]c(=O)oc23)c2nc(N3CCOCC3)cc(=O)n2c1] | 565 |
| 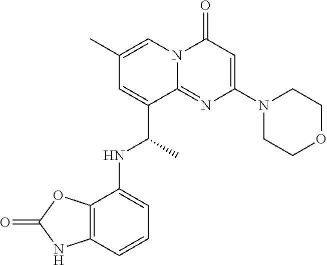 [SMILES: Cc1cc([C@H](C)Nc2cccc3[nH]c(=O)oc23)c2nc(N3CCOCC3)cc(=O)n2c1] | 566 |
| 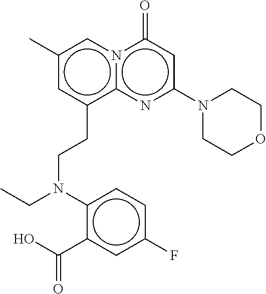 | 567 |
| 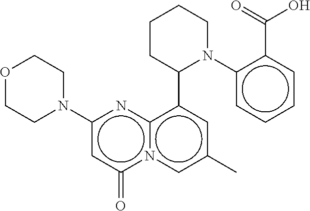 | 568 |
| 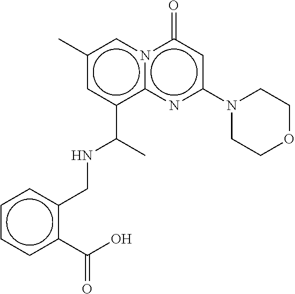 | 569 |
| 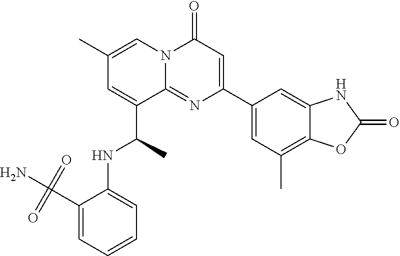 | 570 |
| 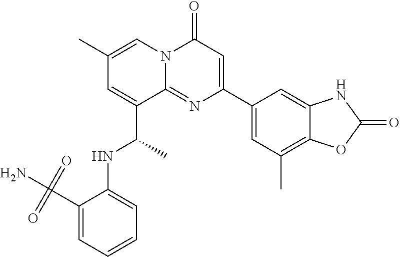 | 571 |
| 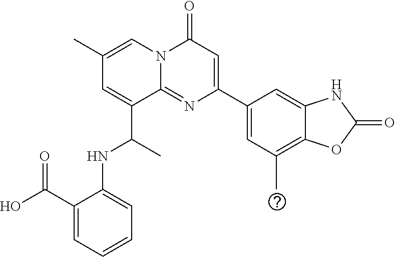 [SMILES: Cc1cc(C(C)Nc2ccccc2C(=O)O)c2nc(-c3cc(C)c4oc(=O)[nH]c4c3)cc(=O)n2c1] | 572 |
| 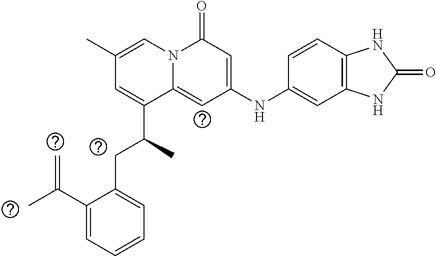 [SMILES: C=C(C)c1ccccc1C[C@H](C)c1cc(C)cn2c(=O)cc(Nc3ccc4[nH]c(=O)[nH]c4c3)cc12] | 573 |
| 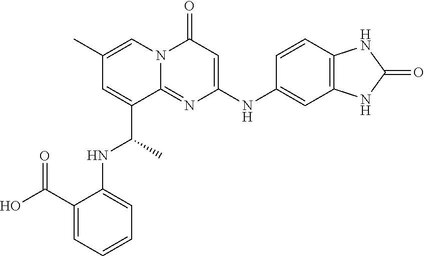 [SMILES: Cc1cc([C@H](C)Nc2ccccc2C(=O)O)c2nc(Nc3ccc4[nH]c(=O)[nH]c4c3)cc(=O)n2c1] | 574 |
| 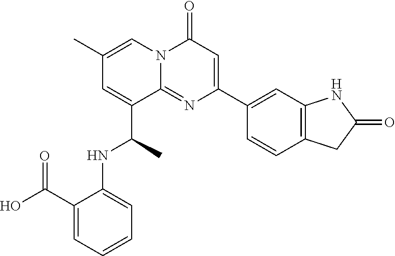 [SMILES: Cc1cc([C@@H](C)Nc2ccccc2C(=O)O)c2nc(-c3ccc4c(c3)NC(=O)C4)cc(=O)n2c1] | 575 |
| 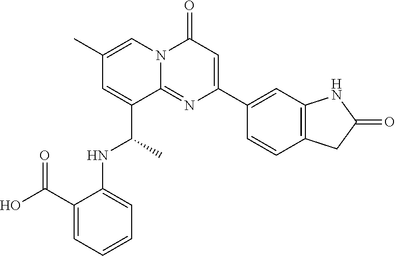 [SMILES: Cc1cc([C@H](C)Nc2ccccc2C(=O)O)c2nc(-c3ccc4c(c3)NC(=O)C4)cc(=O)n2c1] | 576 |
| 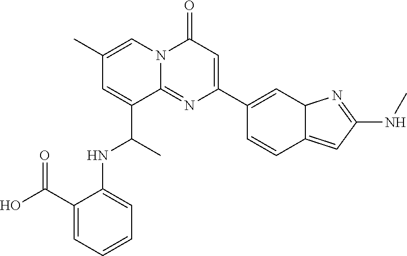 [SMILES: CNC1=NC2C=C(c3cc(=O)n4cc(C)cc(C(C)Nc5ccccc5C(=O)O)c4n3)C=CC2=C1] | 577 |
| 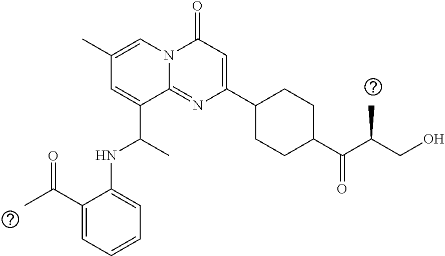 [SMILES: CC(=O)c1ccccc1NC(C)c1cc(C)cn2c(=O)cc(C3CCC(C(=O)[C@@H](C)CO)CC3)nc12] | 578 |
| 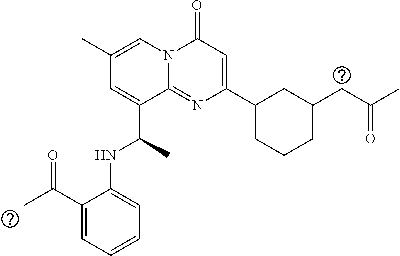 [SMILES: CC(=O)CC1CCCC(c2cc(=O)n3cc(C)cc([C@@H](C)Nc4ccccc4C(C)=O)c3n2)C1] | 579 |
| 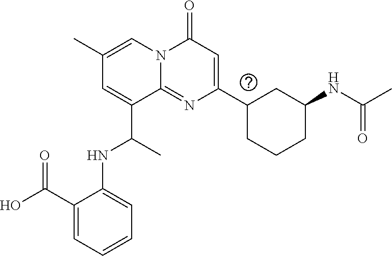 [SMILES: CC(=O)N[C@H]1CCCC(c2cc(=O)n3cc(C)cc(C(C)Nc4ccccc4C(=O)O)c3n2)C1] | 580 |
| 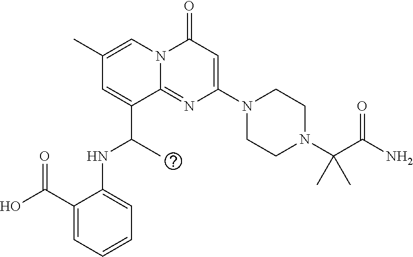 [SMILES: Cc1cc(C(C)Nc2ccccc2C(=O)O)c2nc(N3CCN(C(C)(C)C(N)=O)CC3)cc(=O)n2c1] | 581 |
| 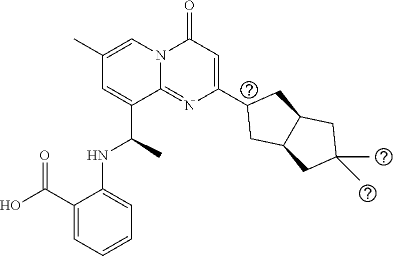 | 582 |
| 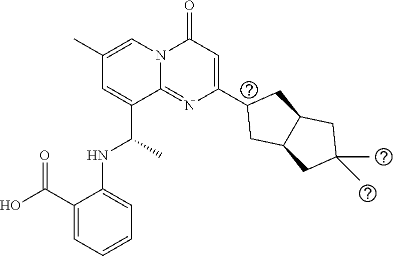 | 583 |
| 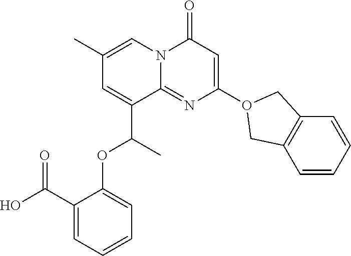 | 584 |
| 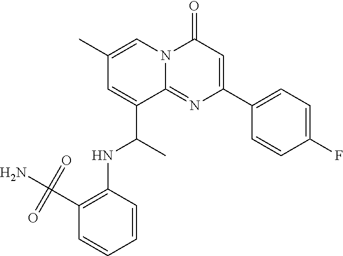 | 585 |
| 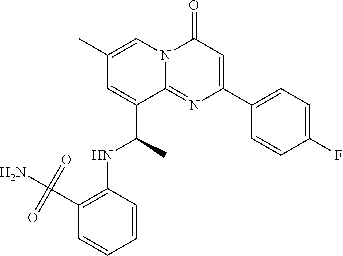 | 586 |
| 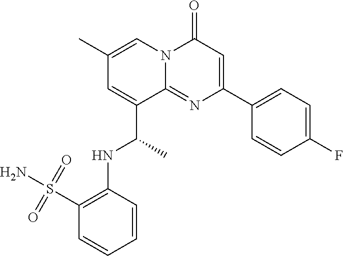 [SMILES: Cc1cc([C@H](C)Nc2ccccc2S(N)(=O)=O)c2nc(-c3ccc(F)cc3)cc(=O)n2c1] | 587 |
| 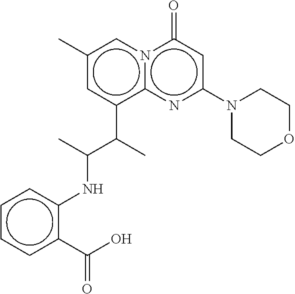 | 588 |
| 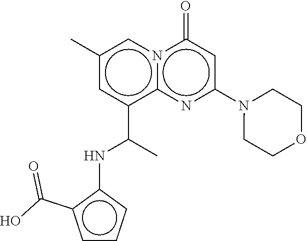 | 589 |
| 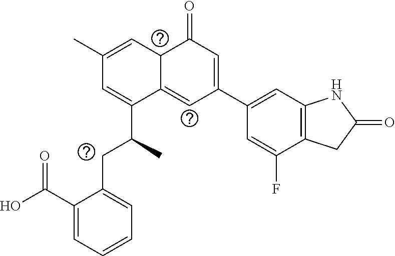 [SMILES: CC1=CC2C(=O)C=C(c3cc(F)c4c(c3)NC(=O)C4)C=C2C([C@@H](C)Cc2ccccc2C(=O)O)=C1] | 590 |
| 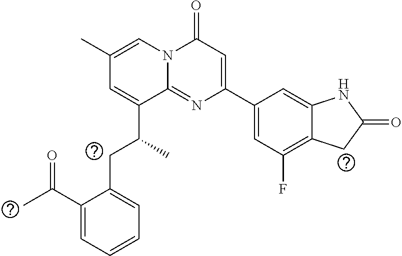 [SMILES: CC(=O)c1ccccc1C[C@@H](C)c1cc(C)cn2c(=O)cc(-c3cc(F)c4c(c3)NC(=O)C4)nc12] | 591 |
| 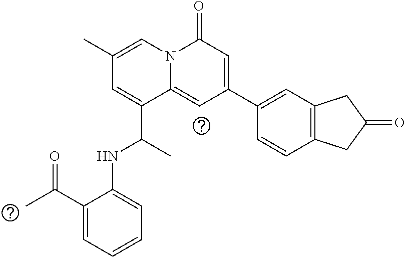 [SMILES: CC(=O)c1ccccc1NC(C)c1cc(C)cn2c(=O)cc(-c3ccc4c(c3)CC(=O)C4)cc12] | 592 |
| 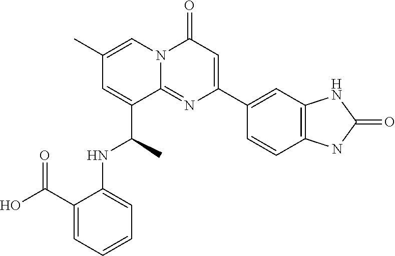 [SMILES: Cc1cc([C@@H](C)Nc2ccccc2C(=O)O)c2nc(-c3ccc4[nH]c(=O)[nH]c4c3)cc(=O)n2c1] | 593 |
| 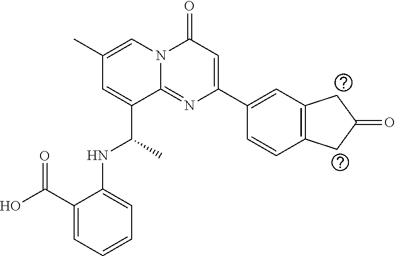 [SMILES: Cc1cc([C@H](C)Nc2ccccc2C(=O)O)c2nc(-c3ccc4c(c3)CC(=O)C4)cc(=O)n2c1] | 594 |
| 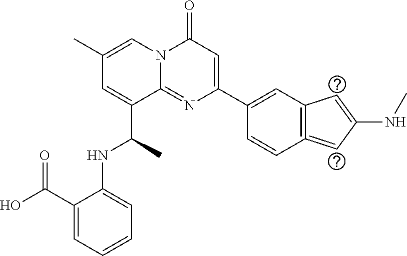 | 595 |
| 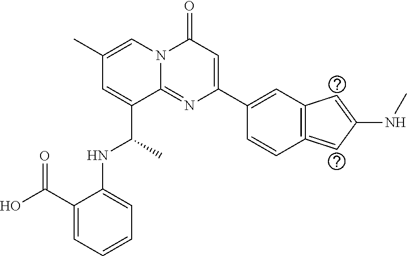 | 596 |
| 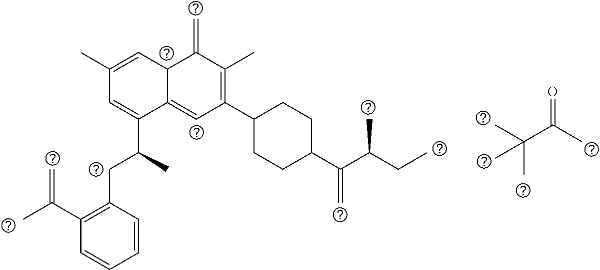 | 597 |
| 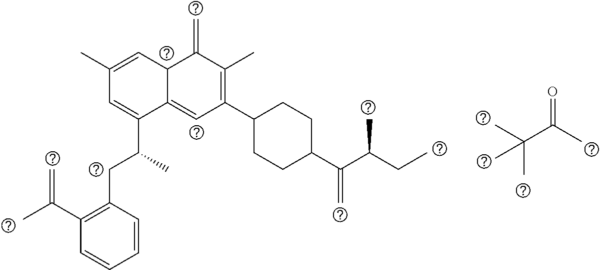 | 598 |
| 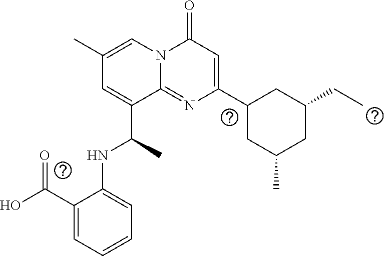 [SMILES: CC[C@H]1CC(c2cc(=O)n3cc(C)cc([C@@H](C)Nc4ccccc4C(=O)O)c3n2)C[C@@H](C)C1] | 599 |
| 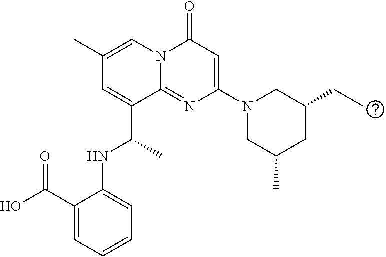 [SMILES: CC[C@@H]1C[C@H](C)CN(c2cc(=O)n3cc(C)cc([C@H](C)Nc4ccccc4C(=O)O)c3n2)C1] | 600 |
| 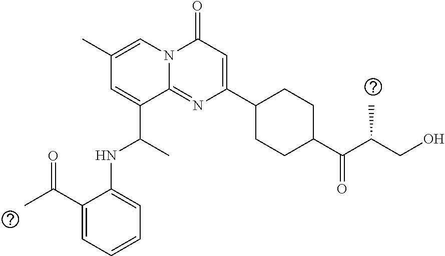 [SMILES: CC(=O)c1ccccc1NC(C)c1cc(C)cn2c(=O)cc(C3CCC(C(=O)[C@H](C)CO)CC3)nc12] | 601 |
| 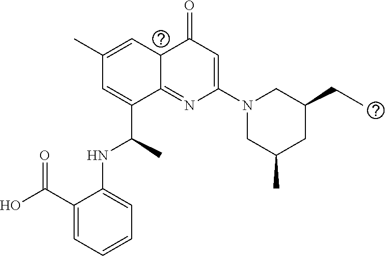 [SMILES: CC[C@H]1C[C@@H](C)CN(C2=CC(=O)C3C=C(C)C=C([C@@H](C)Nc4ccccc4C(=O)O)C3=N2)C1] | 602 |
| 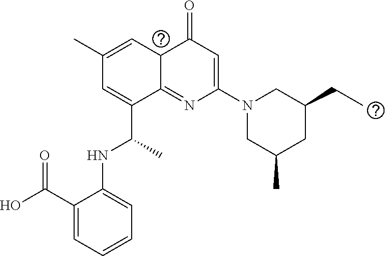 [SMILES: CC[C@H]1C[C@@H](C)CN(C2=CC(=O)C3C=C(C)C=C([C@H](C)Nc4ccccc4C(=O)O)C3=N2)C1] | 603 |
| 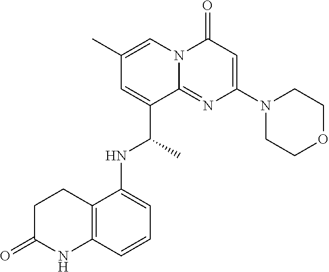 [SMILES: Cc1cc([C@H](C)Nc2cccc3c2CCC(=O)N3)c2nc(N3CCOCC3)cc(=O)n2c1] | 604 |
| 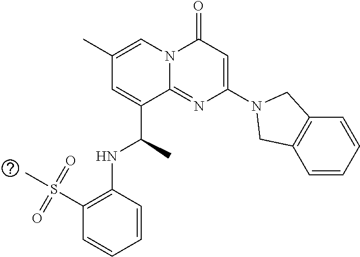 [SMILES: Cc1cc([C@@H](C)Nc2ccccc2S(C)(=O)=O)c2nc(N3Cc4ccccc4C3)cc(=O)n2c1] | 605 |
| 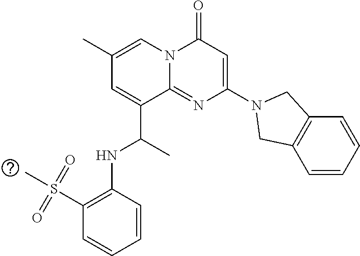 [SMILES: Cc1cc(C(C)Nc2ccccc2S(C)(=O)=O)c2nc(N3Cc4ccccc4C3)cc(=O)n2c1] | 606 |
| 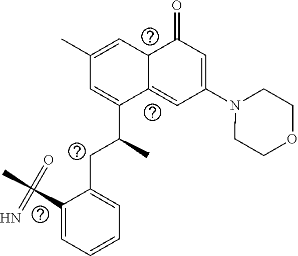 | 607 |
| 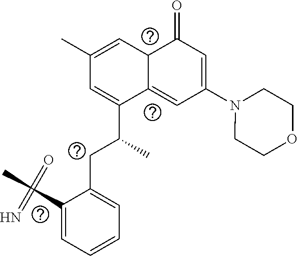 | 608 |
| 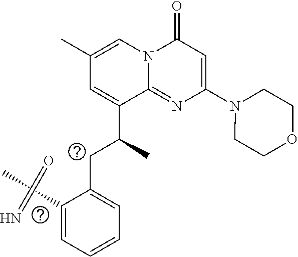 | 609 |
| 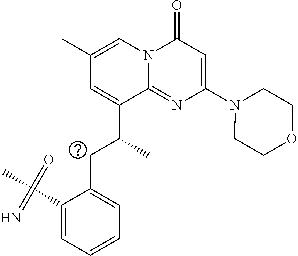 | 610 |
| 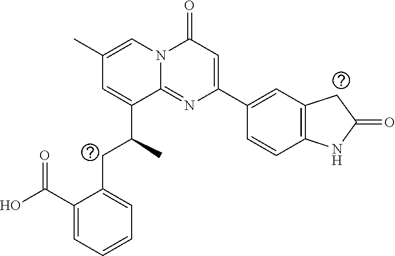 [SMILES: Cc1cc([C@@H](C)Cc2ccccc2C(=O)O)c2nc(-c3ccc4c(c3)CC(=O)N4)cc(=O)n2c1] | 611 |
| 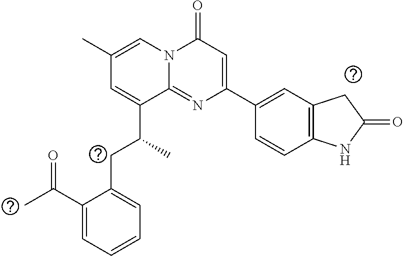 [SMILES: CC(=O)c1ccccc1C[C@@H](C)c1cc(C)cn2c(=O)cc(-c3ccc4c(c3)CC(=O)N4)nc12] | 612 |
| 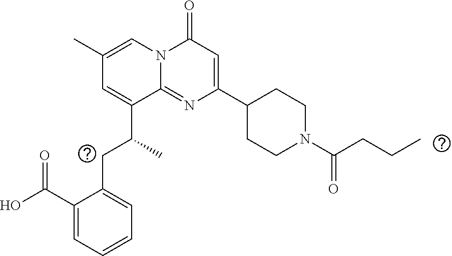 [SMILES: CCCC(=O)N1CCC(c2cc(=O)n3cc(C)cc([C@H](C)Cc4ccccc4C(=O)O)c3n2)CC1] | 613 |
| 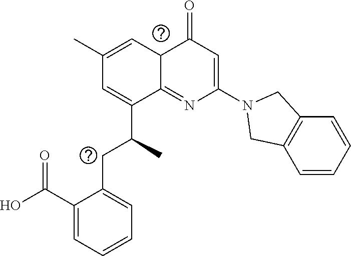 [SMILES: CC1=CC2C(=O)C=C(N3Cc4ccccc4C3)N=C2C([C@@H](C)Cc2ccccc2C(=O)O)=C1] | 614 |
| 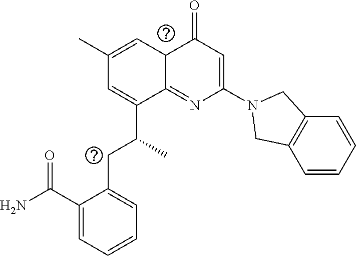 [SMILES: CC1=CC2C(=O)C=C(N3Cc4ccccc4C3)N=C2C([C@H](C)Cc2ccccc2C(N)=O)=C1] | 615 |
| 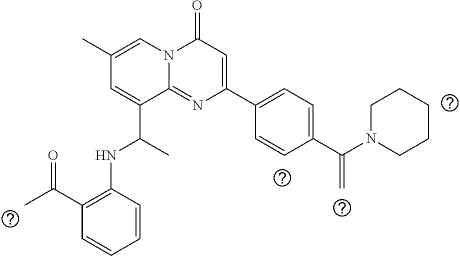 [SMILES: C=C(c1ccc(-c2cc(=O)n3cc(C)cc(C(C)Nc4ccccc4C(C)=O)c3n2)cc1)N1CCCCC1] | 616 |
| 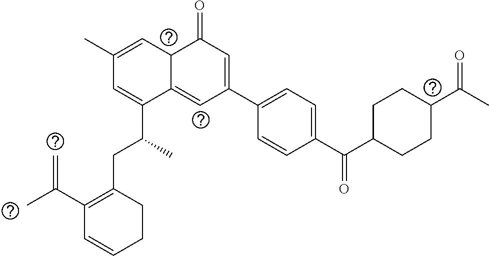 [SMILES: C=C(C)C1=C(C[C@@H](C)C2=CC(C)=CC3C(=O)C=C(c4ccc(C(=O)C5CCC(C(C)=O)CC5)cc4)C=C23)CCC=C1] | 617 |
| 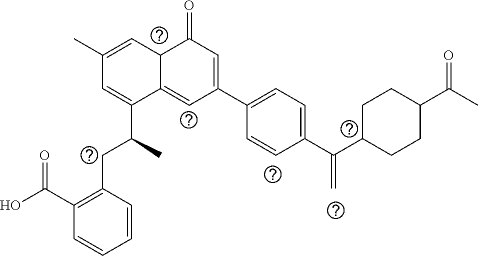 [SMILES: C=C(c1ccc(C2=CC(=O)C3C=C(C)C=C([C@@H](C)Cc4ccccc4C(=O)O)C3=C2)cc1)C1CCC(C(C)=O)CC1] | 618 |
| 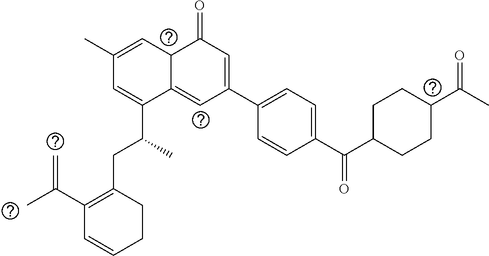 [SMILES: C=C(C)C1=C(C[C@@H](C)C2=CC(C)=CC3C(=O)C=C(c4ccc(C(=O)C5CCC(C(C)=O)CC5)cc4)C=C23)CCC=C1] | 619 |
| 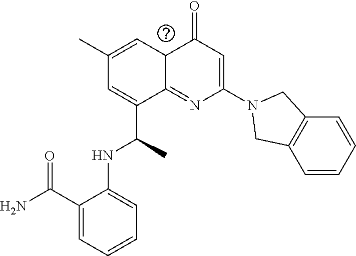 [SMILES: CC1=CC2C(=O)C=C(N3Cc4ccccc4C3)N=C2C([C@@H](C)Nc2ccccc2C(N)=O)=C1] | 620 |
| 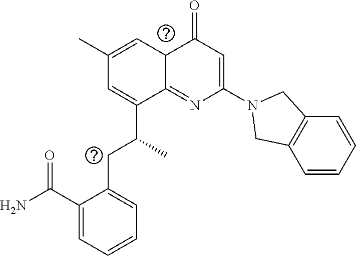 [SMILES: CC1=CC2C(=O)C=C(N3Cc4ccccc4C3)N=C2C([C@H](C)Cc2ccccc2C(N)=O)=C1] | 621 |
| 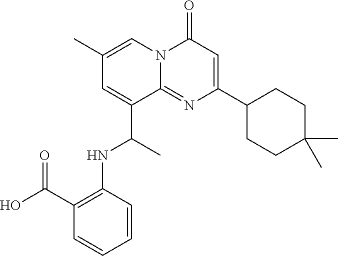 [SMILES: Cc1cc(C(C)Nc2ccccc2C(=O)O)c2nc(C3CCC(C)(C)CC3)cc(=O)n2c1] | 622 |
| 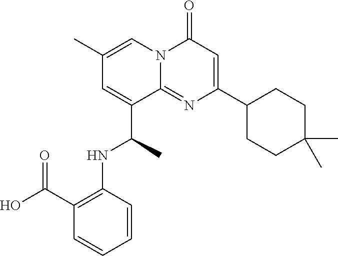 [SMILES: Cc1cc([C@@H](C)Nc2ccccc2C(=O)O)c2nc(C3CCC(C)(C)CC3)cc(=O)n2c1] | 623 |
| 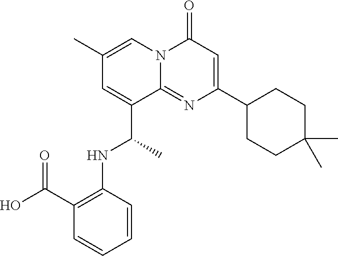 [SMILES: Cc1cc([C@H](C)Nc2ccccc2C(=O)O)c2nc(C3CCC(C)(C)CC3)cc(=O)n2c1] | 624 |
| 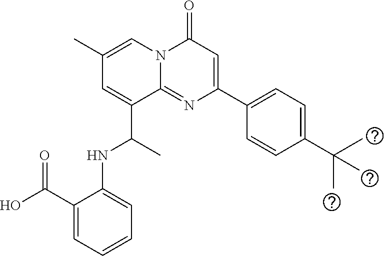 [SMILES: Cc1cc(C(C)Nc2ccccc2C(=O)O)c2nc(-c3ccc(C(C)(C)C)cc3)cc(=O)n2c1] | 625 |
| 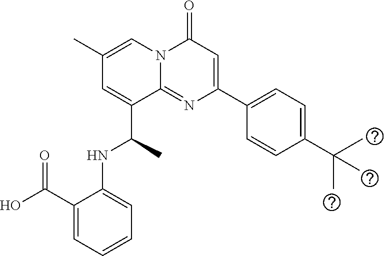 [SMILES: Cc1cc([C@@H](C)Nc2ccccc2C(=O)O)c2nc(-c3ccc(C(C)(C)C)cc3)cc(=O)n2c1] | 626 |
| 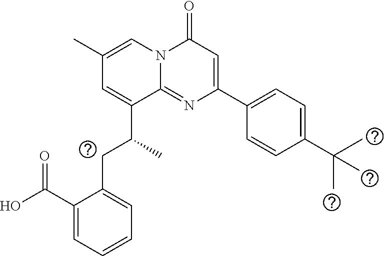 [SMILES: Cc1cc([C@H](C)Cc2ccccc2C(=O)O)c2nc(-c3ccc(C(C)(C)C)cc3)cc(=O)n2c1] | 627 |
| 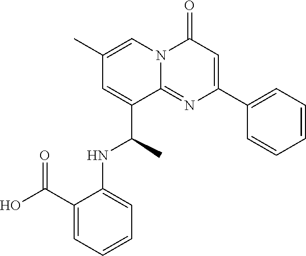 [SMILES: Cc1cc([C@@H](C)Nc2ccccc2C(=O)O)c2nc(-c3ccccc3)cc(=O)n2c1] | 628 |
| 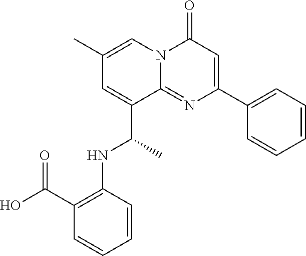 [SMILES: Cc1cc([C@H](C)Nc2ccccc2C(=O)O)c2nc(-c3ccccc3)cc(=O)n2c1] | 629 |
| 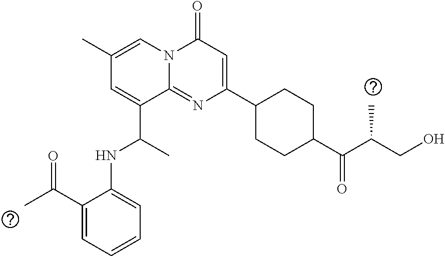 [SMILES: CC(=O)c1ccccc1NC(C)c1cc(C)cn2c(=O)cc(C3CCC(C(=O)[C@H](C)CO)CC3)nc12] | 630 |
| 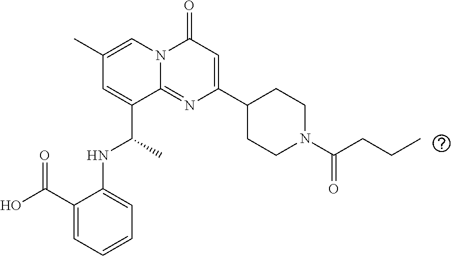 [SMILES: CCCC(=O)N1CCC(c2cc(=O)n3cc(C)cc([C@H](C)Nc4ccccc4C(=O)O)c3n2)CC1] | 631 |
| 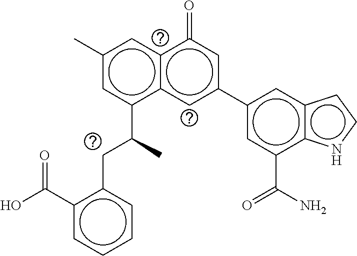 | 632 |
| 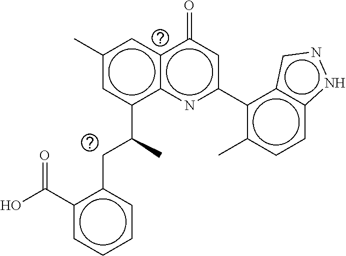 | 633 |
| 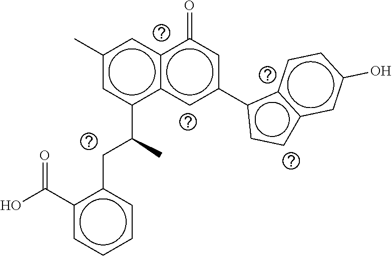 | 634 |
| 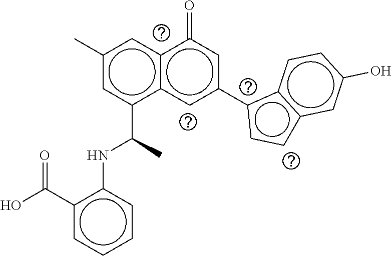 | 635 |
| 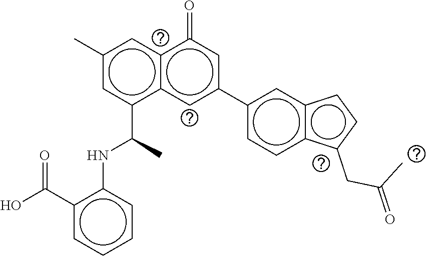 | 636 |
| 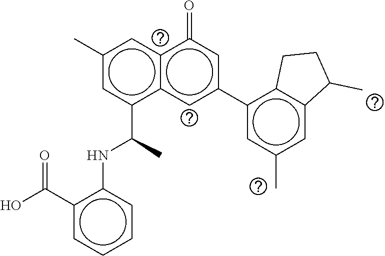 | 637 |
| 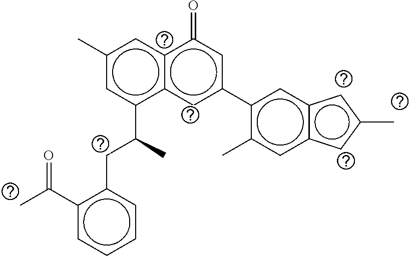 | 638 |
| 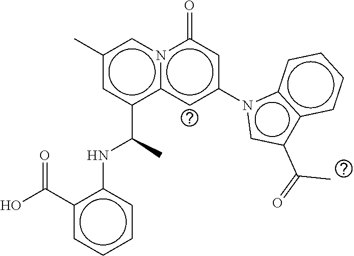 | 639 |
| 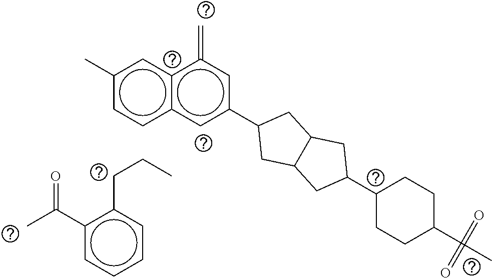 | 640 |
| 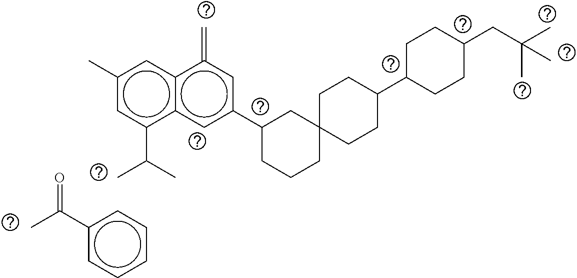 | 641 |
| 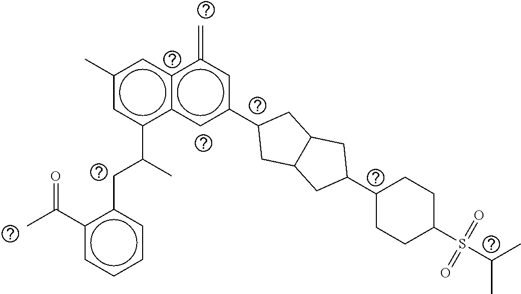 | 642 |
| 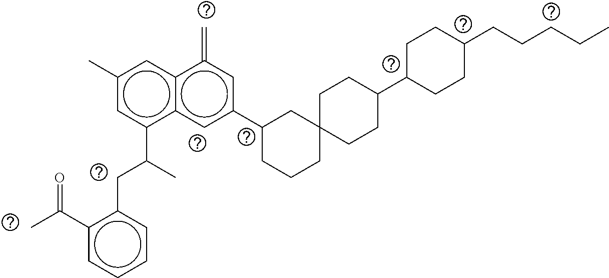 | 643 |
| 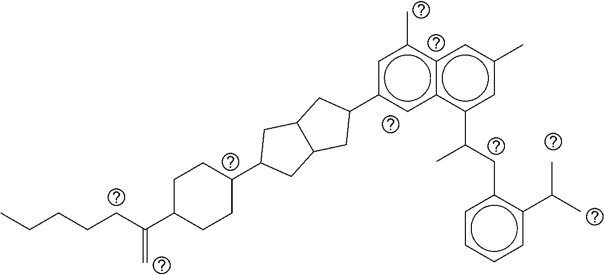 | 644 |
| 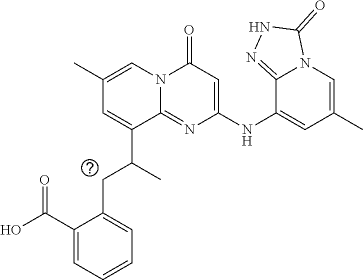 [SMILES: Cc1cc(C(C)Cc2ccccc2C(=O)O)c2nc(Nc3cc(C)cn4c(=O)[nH]nc34)cc(=O)n2c1] | 645 |
| 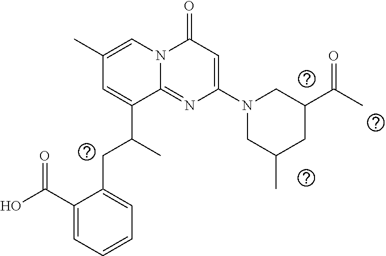 [SMILES: CC(=O)C1CC(C)CN(c2cc(=O)n3cc(C)cc(C(C)Cc4ccccc4C(=O)O)c3n2)C1] | 646 |
| 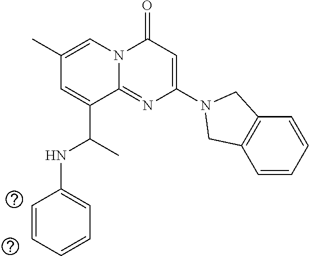 [SMILES: Cc1cc(C(C)Nc2ccccc2)c2nc(N3Cc4ccccc4C3)cc(=O)n2c1] | 647 |
| 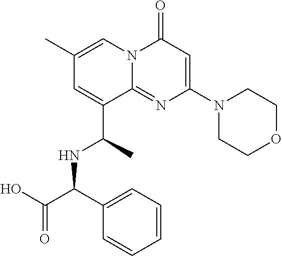 [SMILES: Cc1cc([C@@H](C)N[C@H](C(=O)O)c2ccccc2)c2nc(N3CCOCC3)cc(=O)n2c1] | 648 |
| 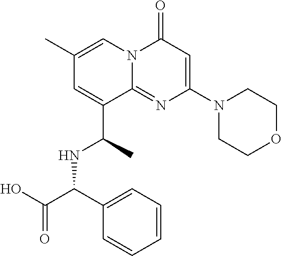 [SMILES: Cc1cc([C@@H](C)N[C@@H](C(=O)O)c2ccccc2)c2nc(N3CCOCC3)cc(=O)n2c1] | 649 |
| 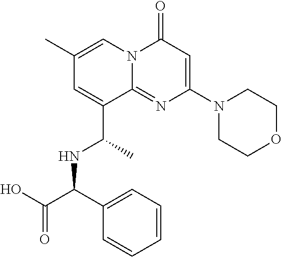 [SMILES: Cc1cc([C@H](C)N[C@H](C(=O)O)c2ccccc2)c2nc(N3CCOCC3)cc(=O)n2c1] | 650 |
| 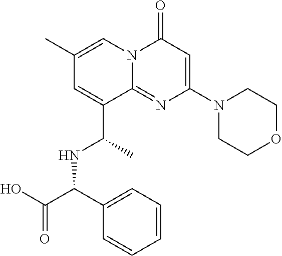 [SMILES: Cc1cc([C@H](C)N[C@@H](C(=O)O)c2ccccc2)c2nc(N3CCOCC3)cc(=O)n2c1] | 651 |
| 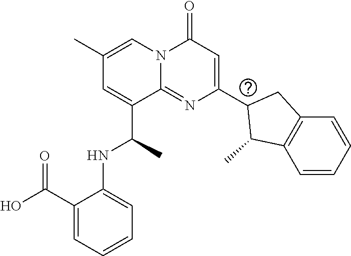 [SMILES: Cc1cc([C@@H](C)Nc2ccccc2C(=O)O)c2nc(C3Cc4ccccc4[C@@H]3C)cc(=O)n2c1] | 652 |
| 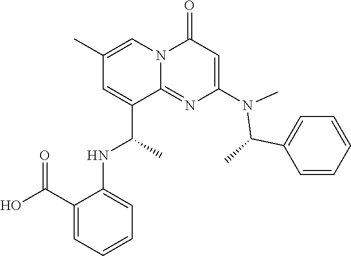 [SMILES: Cc1cc([C@H](C)Nc2ccccc2C(=O)O)c2nc(N(C)[C@@H](C)c3ccccc3)cc(=O)n2c1] | 653 |
| 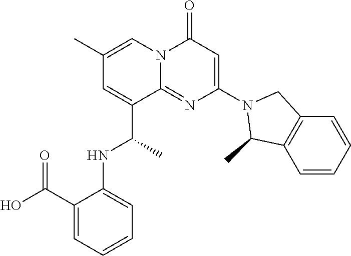 [SMILES: Cc1cc([C@H](C)Nc2ccccc2C(=O)O)c2nc(N3Cc4ccccc4[C@H]3C)cc(=O)n2c1] | 654 |
| 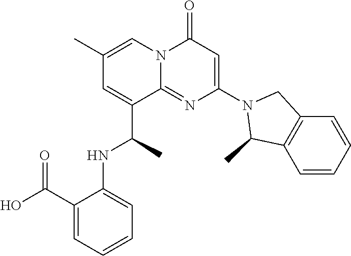 [SMILES: Cc1cc([C@@H](C)Nc2ccccc2C(=O)O)c2nc(N3Cc4ccccc4[C@H]3C)cc(=O)n2c1] | 655 |
| 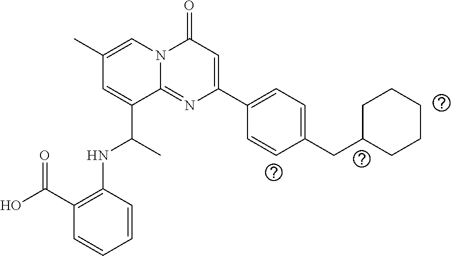 [SMILES: Cc1cc(C(C)Nc2ccccc2C(=O)O)c2nc(-c3ccc(CC4CCCCC4)cc3)cc(=O)n2c1] | 656 |
| 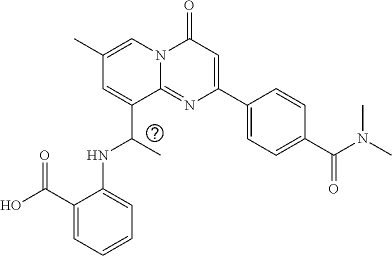 [SMILES: Cc1cc(C(C)Nc2ccccc2C(=O)O)c2nc(-c3ccc(C(=O)N(C)C)cc3)cc(=O)n2c1] | 657 |
| 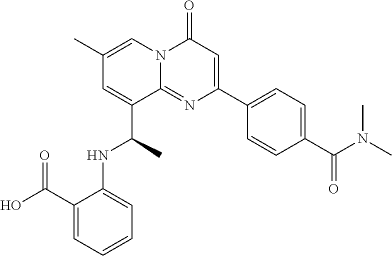 [SMILES: Cc1cc([C@@H](C)Nc2ccccc2C(=O)O)c2nc(-c3ccc(C(=O)N(C)C)cc3)cc(=O)n2c1] | 658 |
| 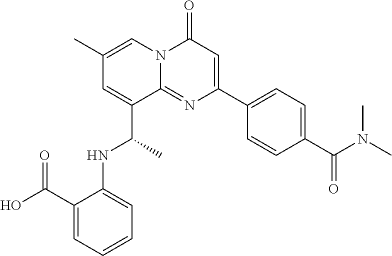 [SMILES: Cc1cc([C@H](C)Nc2ccccc2C(=O)O)c2nc(-c3ccc(C(=O)N(C)C)cc3)cc(=O)n2c1] | 659 |
| 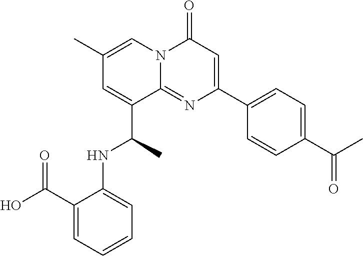 [SMILES: CC(=O)c1ccc(-c2cc(=O)n3cc(C)cc([C@@H](C)Nc4ccccc4C(=O)O)c3n2)cc1] | 660 |
| 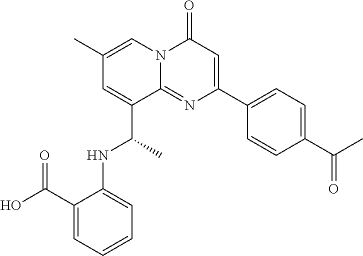 [SMILES: CC(=O)c1ccc(-c2cc(=O)n3cc(C)cc([C@H](C)Nc4ccccc4C(=O)O)c3n2)cc1] | 661 |
| 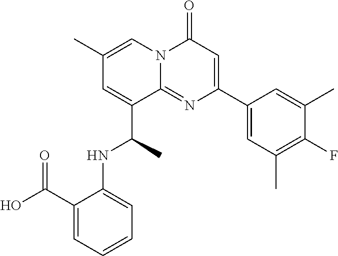 [SMILES: Cc1cc([C@@H](C)Nc2ccccc2C(=O)O)c2nc(-c3cc(C)c(F)c(C)c3)cc(=O)n2c1] | 662 |
| 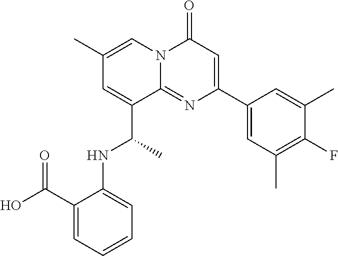 [SMILES: Cc1cc([C@H](C)Nc2ccccc2C(=O)O)c2nc(-c3cc(C)c(F)c(C)c3)cc(=O)n2c1] | 663 |
| 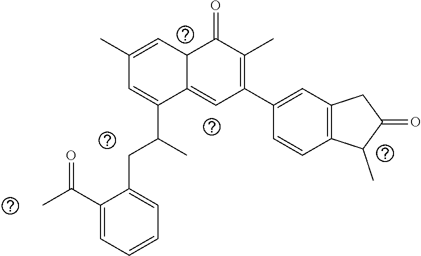 [SMILES: CC(=O)c1ccccc1CC(C)C1=CC(C)=CC2C(=O)C(C)=C(c3ccc4c(c3)CC(=O)C4C)C=C12] | 664 |
| 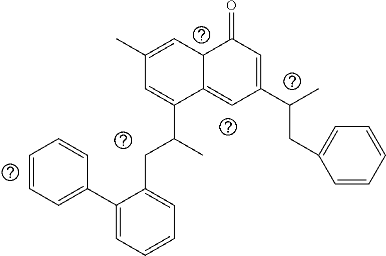 [SMILES: CC1=CC2C(=O)C=C(C(C)Cc3ccccc3)C=C2C(C(C)Cc2ccccc2-c2ccccc2)=C1] | 665 |
| 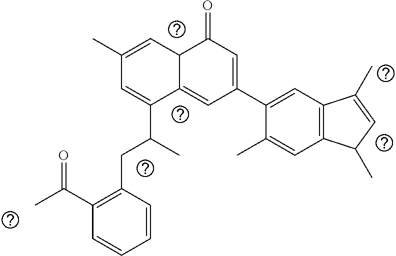 [SMILES: CC(=O)c1ccccc1CC(C)C1=CC(C)=CC2C(=O)C=C(c3cc4c(cc3C)C(C)C=C4C)C=C12] | 666 |
| 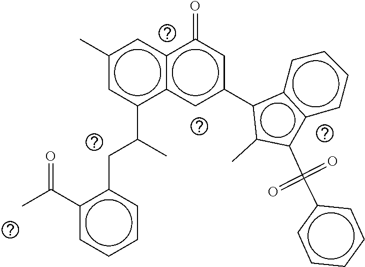 | 667 |
| 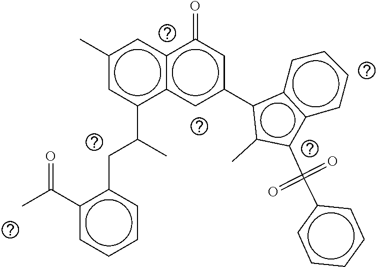 | 668 |
| 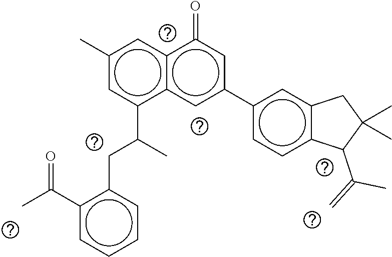 | 669 |
| 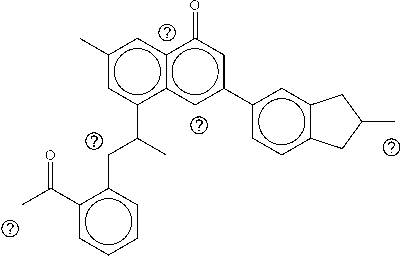 | 670 |
| 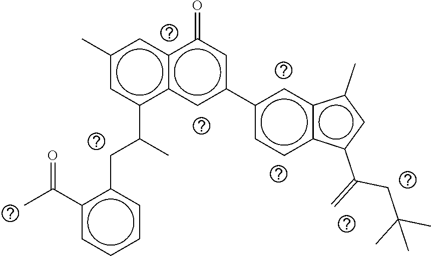 | 671 |
| 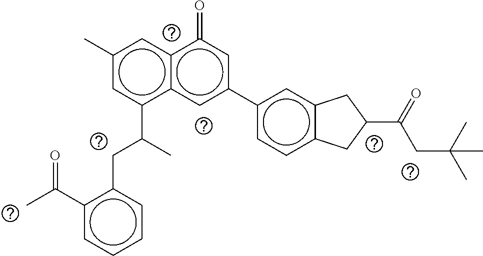 | 672 |
| 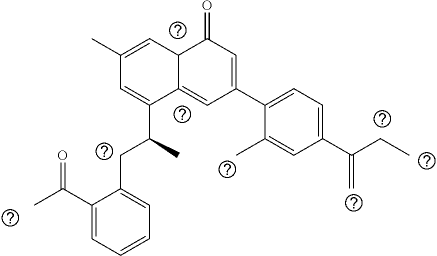 [SMILES: C=C(CC)c1ccc(C2=CC(=O)C3C=C(C)C=C([C@@H](C)Cc4ccccc4C(C)=O)C3=C2)c(C)c1] | 673 |
| 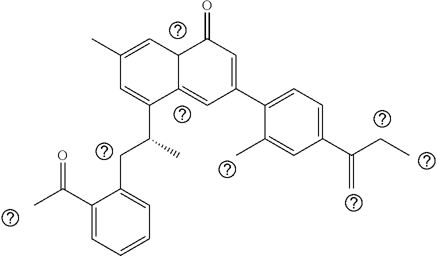 [SMILES: C=C(CC)c1ccc(C2=CC(=O)C3C=C(C)C=C([C@H](C)Cc4ccccc4C(C)=O)C3=C2)c(C)c1] | 674 |
| 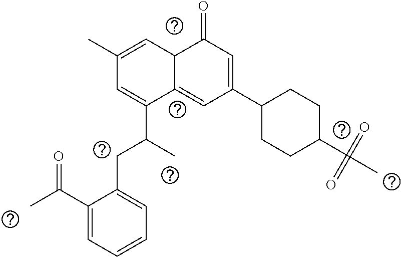 | 675 |
| 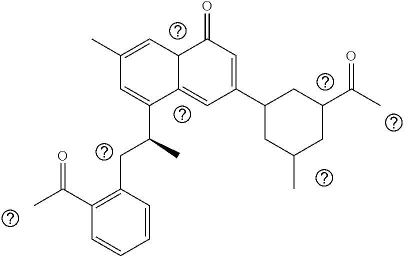 [SMILES: CC(=O)c1ccccc1C[C@H](C)C1=CC(C)=CC2C(=O)C=C(C3CC(C)CC(C(C)=O)C3)C=C12] | 676 |
| 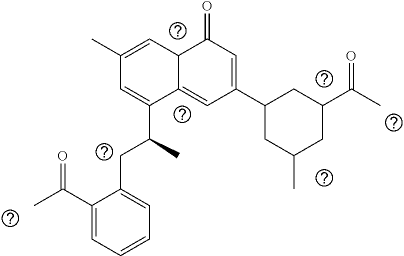 [SMILES: CC(=O)c1ccccc1C[C@H](C)C1=CC(C)=CC2C(=O)C=C(C3CC(C)CC(C(C)=O)C3)C=C12] | 677 |
| 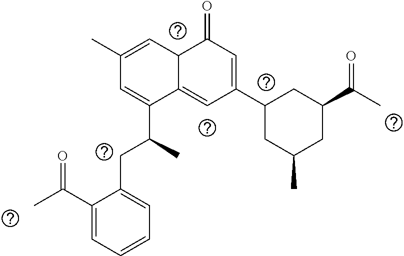 [SMILES: CC(=O)c1ccccc1C[C@H](C)C1=CC(C)=CC2C(=O)C=C(C3C[C@H](C)C[C@H](C(C)=O)C3)C=C12] | 678 |
| 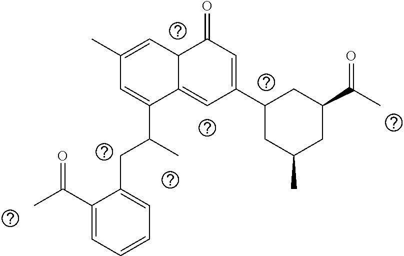 [SMILES: CC(=O)c1ccccc1CC(C)C1=CC(C)=CC2C(=O)C=C(C3C[C@H](C)C[C@H](C(C)=O)C3)C=C12] | 679 |
| 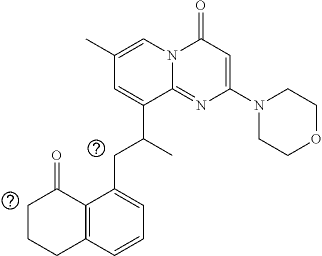 [SMILES: Cc1cc(C(C)Cc2cccc3c2C(=O)CCC3)c2nc(N3CCOCC3)cc(=O)n2c1] | 680 |
| 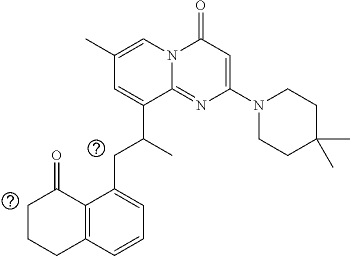 [SMILES: Cc1cc(C(C)Cc2cccc3c2C(=O)CCC3)c2nc(N3CCC(C)(C)CC3)cc(=O)n2c1] | 681 |
| 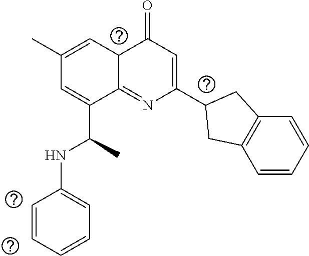 [SMILES: CC1=CC2C(=O)C=C(C3Cc4ccccc4C3)N=C2C([C@@H](C)Nc2ccccc2)=C1] | 682 |
| 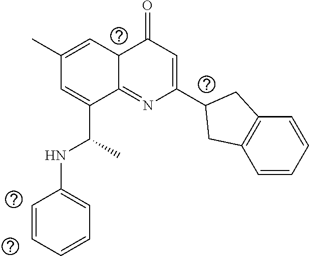 [SMILES: CC1=CC2C(=O)C=C(C3Cc4ccccc4C3)N=C2C([C@H](C)Nc2ccccc2)=C1] | 683 |
| 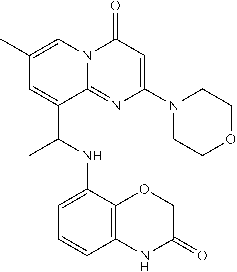 [SMILES: Cc1cc(C(C)Nc2cccc3c2OCC(=O)N3)c2nc(N3CCOCC3)cc(=O)n2c1] | 684 |
| 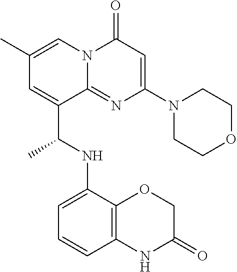 [SMILES: Cc1cc([C@@H](C)Nc2cccc3c2OCC(=O)N3)c2nc(N3CCOCC3)cc(=O)n2c1] | 685 |
| 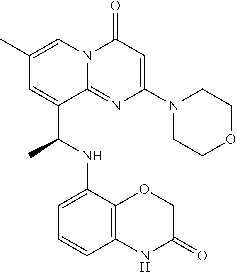 [SMILES: Cc1cc([C@H](C)Nc2cccc3c2OCC(=O)N3)c2nc(N3CCOCC3)cc(=O)n2c1] | 686 |
| 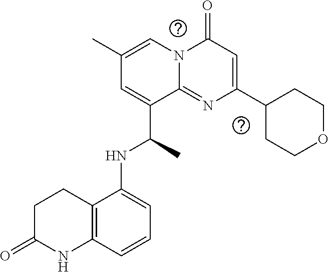 [SMILES: Cc1cc([C@@H](C)Nc2cccc3c2CCC(=O)N3)c2nc(C3CCOCC3)cc(=O)n2c1] | 687 |
| 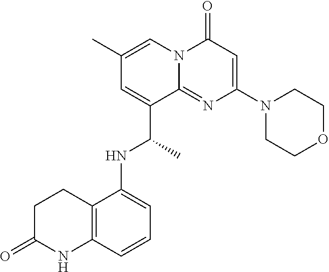 [SMILES: Cc1cc([C@H](C)Nc2cccc3c2CCC(=O)N3)c2nc(N3CCOCC3)cc(=O)n2c1] | 688 |
| 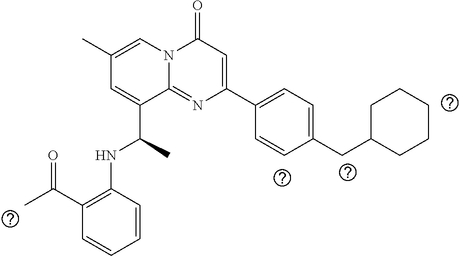 [SMILES: CC(=O)c1ccccc1N[C@H](C)c1cc(C)cn2c(=O)cc(-c3ccc(CC4CCCCC4)cc3)nc12] | 689 |
| 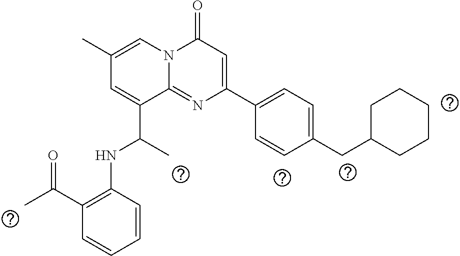 [SMILES: CC(=O)c1ccccc1NC(C)c1cc(C)cn2c(=O)cc(-c3ccc(CC4CCCCC4)cc3)nc12] | 690 |
| 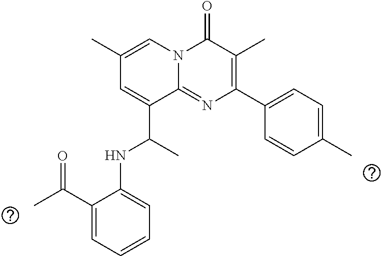 [SMILES: CC(=O)c1ccccc1NC(C)c1cc(C)cn2c(=O)c(C)c(-c3ccc(C)cc3)nc12] | 691 |
| 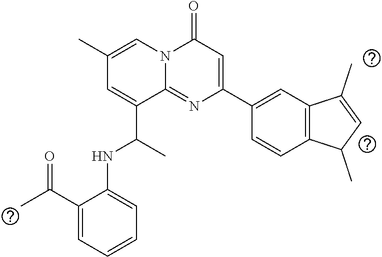 [SMILES: CC(=O)c1ccccc1NC(C)c1cc(C)cn2c(=O)cc(-c3ccc4c(c3)C(C)=CC4C)nc12] | 692 |
| 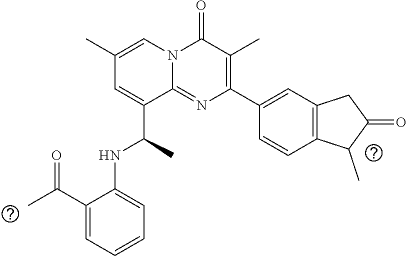 [SMILES: CC(=O)c1ccccc1N[C@H](C)c1cc(C)cn2c(=O)c(C)c(-c3ccc4c(c3)CC(=O)C4C)nc12] | 693 |
| 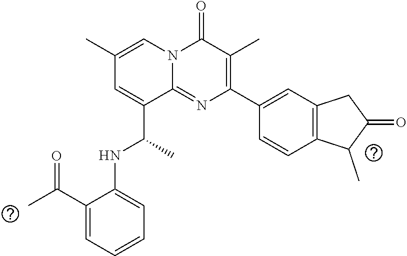 [SMILES: CC(=O)c1ccccc1N[C@@H](C)c1cc(C)cn2c(=O)c(C)c(-c3ccc4c(c3)CC(=O)C4C)nc12] | 694 |
| 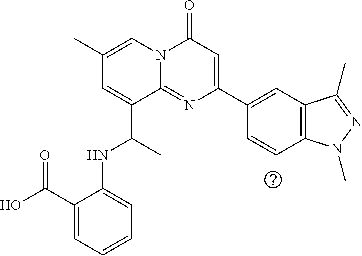 [SMILES: Cc1cc(C(C)Nc2ccccc2C(=O)O)c2nc(-c3ccc4c(c3)c(C)nn4C)cc(=O)n2c1] | 695 |
| 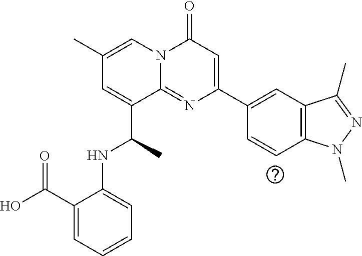 [SMILES: Cc1cc([C@@H](C)Nc2ccccc2C(=O)O)c2nc(-c3ccc4c(c3)c(C)nn4C)cc(=O)n2c1] | 696 |
| 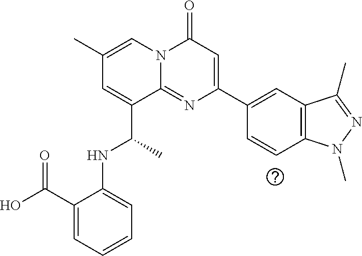 [SMILES: Cc1cc([C@H](C)Nc2ccccc2C(=O)O)c2nc(-c3ccc4c(c3)c(C)nn4C)cc(=O)n2c1] | 697 |
| 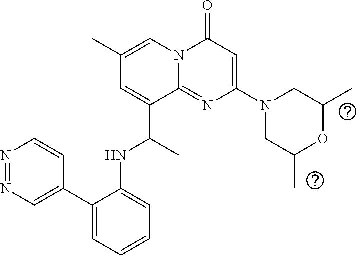 [SMILES: Cc1cc(C(C)Nc2ccccc2-c2ccnnc2)c2nc(N3CC(C)OC(C)C3)cc(=O)n2c1] | 698 |
| 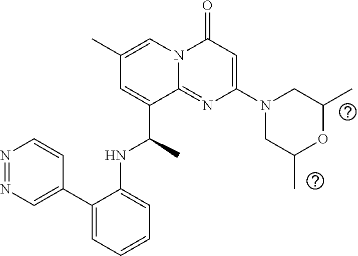 [SMILES: Cc1cc([C@@H](C)Nc2ccccc2-c2ccnnc2)c2nc(N3CC(C)OC(C)C3)cc(=O)n2c1] | 699 |
| 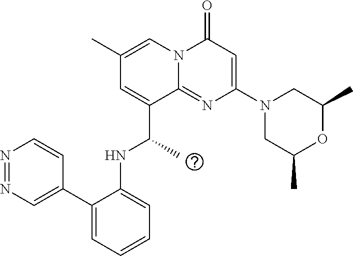 [SMILES: Cc1cc([C@H](C)Nc2ccccc2-c2ccnnc2)c2nc(N3C[C@@H](C)O[C@@H](C)C3)cc(=O)n2c1] | 700 |
| 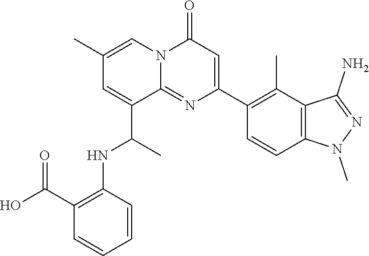 [SMILES: Cc1cc(C(C)Nc2ccccc2C(=O)O)c2nc(-c3ccc4c(c(N)nn4C)c3C)cc(=O)n2c1] | 701 |
| 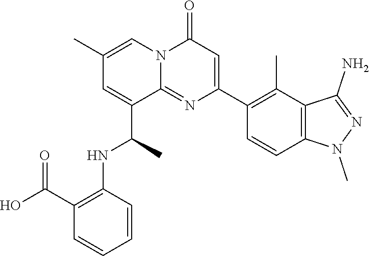 [SMILES: Cc1cc([C@@H](C)Nc2ccccc2C(=O)O)c2nc(-c3ccc4c(c(N)nn4C)c3C)cc(=O)n2c1] | 702 |
| 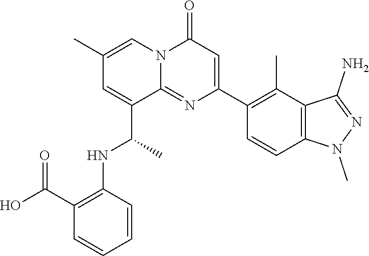 [SMILES: Cc1cc([C@H](C)Nc2ccccc2C(=O)O)c2nc(-c3ccc4c(c(N)nn4C)c3C)cc(=O)n2c1] | 703 |
| 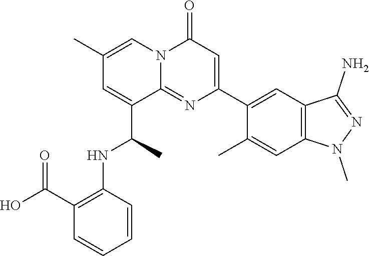 [SMILES: Cc1cc([C@@H](C)Nc2ccccc2C(=O)O)c2nc(-c3cc4c(N)nn(C)c4cc3C)cc(=O)n2c1] | 704 |
| 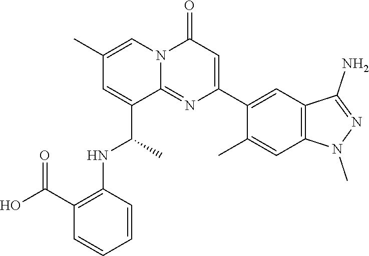 [SMILES: Cc1cc([C@H](C)Nc2ccccc2C(=O)O)c2nc(-c3cc4c(N)nn(C)c4cc3C)cc(=O)n2c1] | 705 |
| 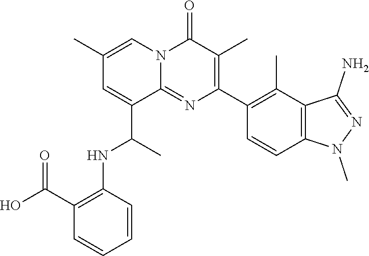 [SMILES: Cc1cc(C(C)Nc2ccccc2C(=O)O)c2nc(-c3ccc4c(c(N)nn4C)c3C)c(C)c(=O)n2c1] | 706 |
| 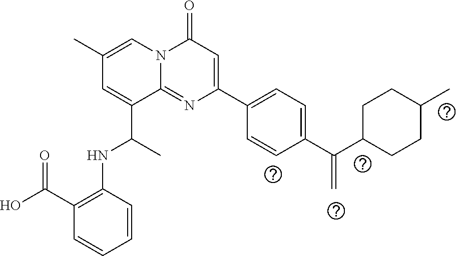 [SMILES: C=C(c1ccc(-c2cc(=O)n3cc(C)cc(C(C)Nc4ccccc4C(=O)O)c3n2)cc1)C1CCC(C)CC1] | 707 |
| 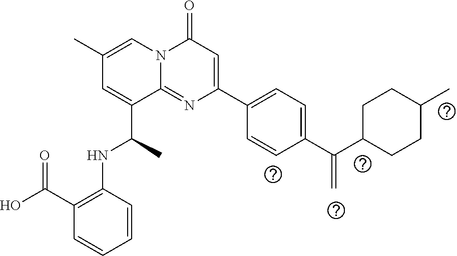 [SMILES: C=C(c1ccc(-c2cc(=O)n3cc(C)cc([C@@H](C)Nc4ccccc4C(=O)O)c3n2)cc1)C1CCC(C)CC1] | 708 |
| 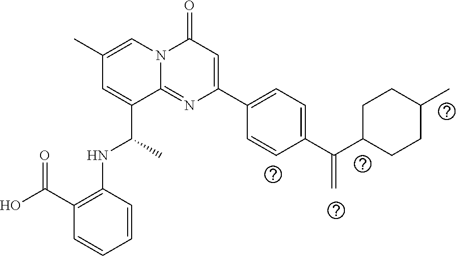 [SMILES: C=C(c1ccc(-c2cc(=O)n3cc(C)cc([C@H](C)Nc4ccccc4C(=O)O)c3n2)cc1)C1CCC(C)CC1] | 709 |
| 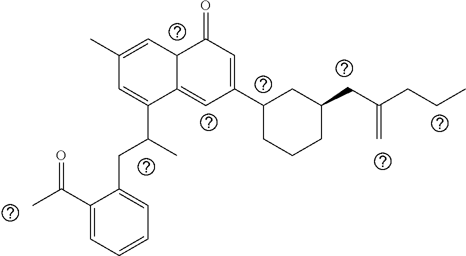 [SMILES: C=C(CCC)C[C@H]1CCCC(C2=CC(=O)C3C=C(C)C=C(C(C)Cc4ccccc4C(C)=O)C3=C2)C1] | 710 |
| 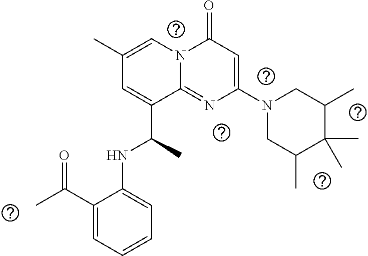 [SMILES: CC(=O)c1ccccc1N[C@H](C)c1cc(C)cn2c(=O)cc(N3CC(C)C(C)(C)C(C)C3)nc12] | 711 |
| 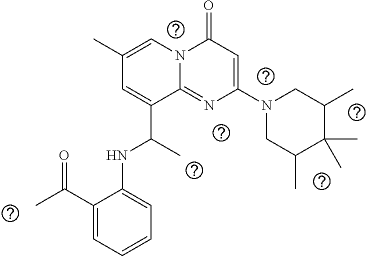 [SMILES: CC(=O)c1ccccc1NC(C)c1cc(C)cn2c(=O)cc(N3CC(C)C(C)(C)C(C)C3)nc12] | 712 |
| 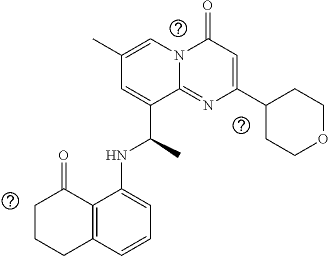 [SMILES: Cc1cc([C@@H](C)Nc2cccc3c2C(=O)CCC3)c2nc(C3CCOCC3)cc(=O)n2c1] | 713 |
| 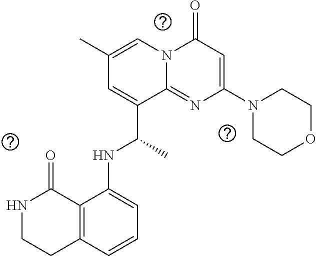 [SMILES: Cc1cc([C@H](C)Nc2cccc3c2C(=O)NCC3)c2nc(N3CCOCC3)cc(=O)n2c1] | 714 |
| 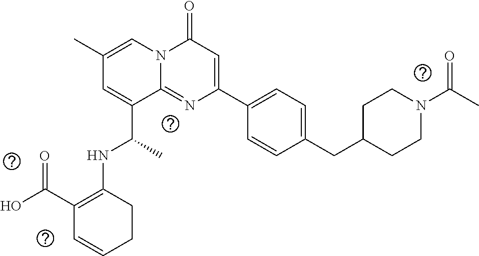 [SMILES: CC(=O)N1CCC(Cc2ccc(-c3cc(=O)n4cc(C)cc([C@H](C)NC5=C(C(=O)O)C=CCC5)c4n3)cc2)CC1] | 715 |
| 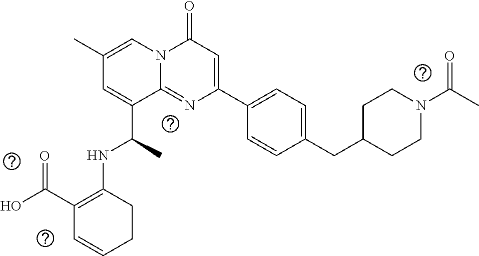 [SMILES: CC(=O)N1CCC(Cc2ccc(-c3cc(=O)n4cc(C)cc([C@@H](C)NC5=C(C(=O)O)C=CCC5)c4n3)cc2)CC1] | 716 |
| 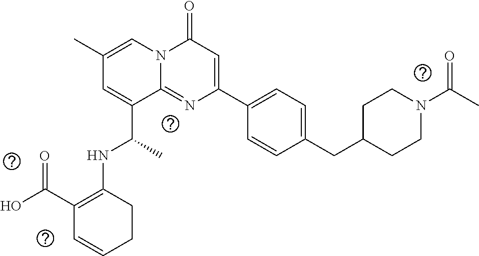 [SMILES: CC(=O)N1CCC(Cc2ccc(-c3cc(=O)n4cc(C)cc([C@H](C)NC5=C(C(=O)O)C=CCC5)c4n3)cc2)CC1] | 717 |
| 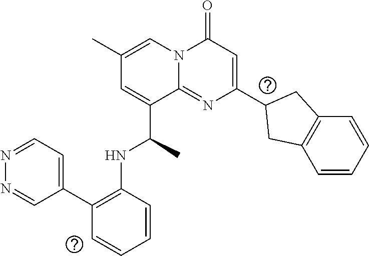 [SMILES: Cc1cc([C@@H](C)Nc2ccccc2-c2ccnnc2)c2nc(C3Cc4ccccc4C3)cc(=O)n2c1] | 718 |
| 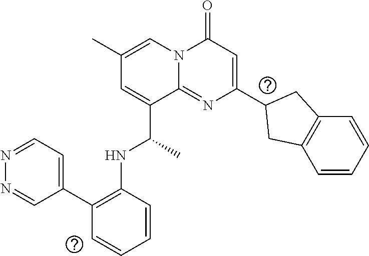 [SMILES: Cc1cc([C@H](C)Nc2ccccc2-c2ccnnc2)c2nc(C3Cc4ccccc4C3)cc(=O)n2c1] | 719 |
| 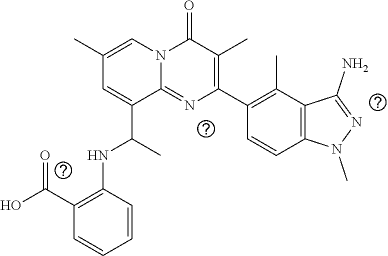 [SMILES: Cc1cc(C(C)Nc2ccccc2C(=O)O)c2nc(-c3ccc4c(c(N)nn4C)c3C)c(C)c(=O)n2c1] | 720 |
| 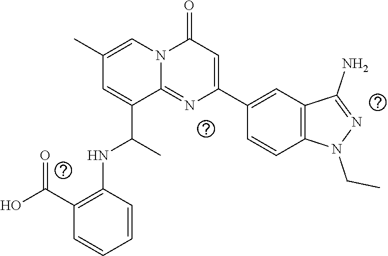 [SMILES: CCn1nc(N)c2cc(-c3cc(=O)n4cc(C)cc(C(C)Nc5ccccc5C(=O)O)c4n3)ccc21] | 721 |
| 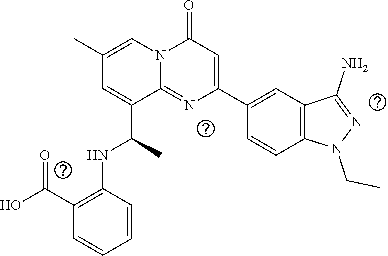 [SMILES: CCn1nc(N)c2cc(-c3cc(=O)n4cc(C)cc([C@@H](C)Nc5ccccc5C(=O)O)c4n3)ccc21] | 722 |
| 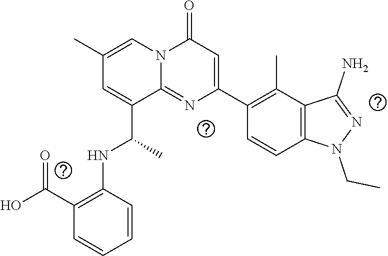 [SMILES: CCn1nc(N)c2c(C)c(-c3cc(=O)n4cc(C)cc([C@H](C)Nc5ccccc5C(=O)O)c4n3)ccc21] | 723 |
| 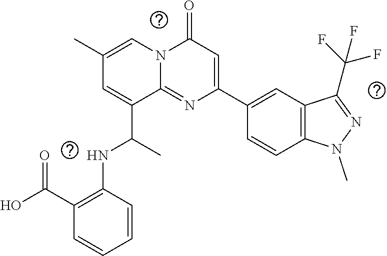 [SMILES: Cc1cc(C(C)Nc2ccccc2C(=O)O)c2nc(-c3ccc4c(c3)c(C(F)(F)F)nn4C)cc(=O)n2c1] | 724 |
| 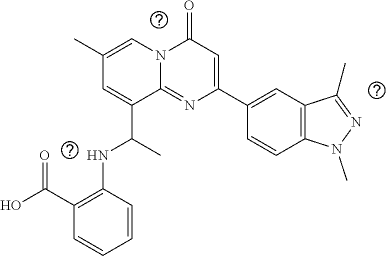 [SMILES: Cc1cc(C(C)Nc2ccccc2C(=O)O)c2nc(-c3ccc4c(c3)c(C)nn4C)cc(=O)n2c1] | 725 |
| 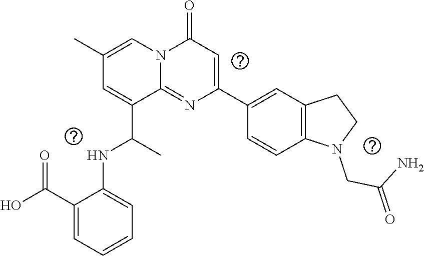 [SMILES: Cc1cc(C(C)Nc2ccccc2C(=O)O)c2nc(-c3ccc4c(c3)CCN4CC(N)=O)cc(=O)n2c1] | 726 |
| 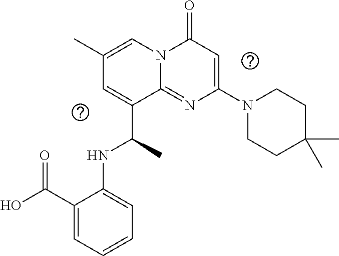 [SMILES: Cc1cc([C@@H](C)Nc2ccccc2C(=O)O)c2nc(N3CCC(C)(C)CC3)cc(=O)n2c1] | 727 |
| 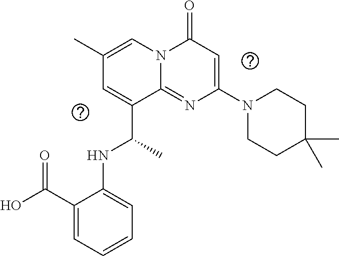 [SMILES: Cc1cc([C@H](C)Nc2ccccc2C(=O)O)c2nc(N3CCC(C)(C)CC3)cc(=O)n2c1] | 728 |
| 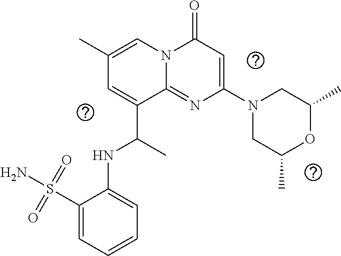 [SMILES: Cc1cc(C(C)Nc2ccccc2S(N)(=O)=O)c2nc(N3C[C@@H](C)O[C@@H](C)C3)cc(=O)n2c1] | 729 |
| 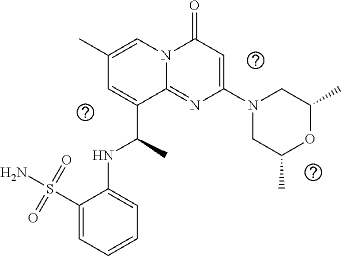 [SMILES: Cc1cc([C@@H](C)Nc2ccccc2S(N)(=O)=O)c2nc(N3C[C@@H](C)O[C@@H](C)C3)cc(=O)n2c1] | 730 |
| 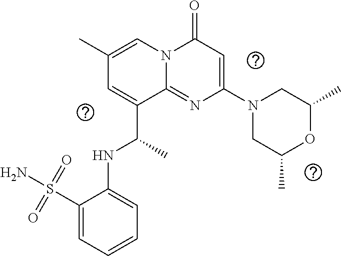 [SMILES: Cc1cc([C@H](C)Nc2ccccc2S(N)(=O)=O)c2nc(N3C[C@@H](C)O[C@@H](C)C3)cc(=O)n2c1] | 731 |
| 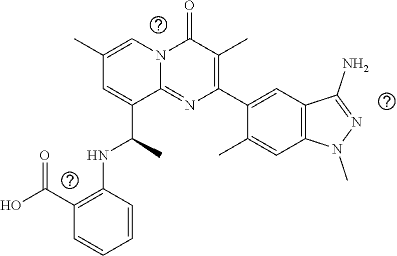 [SMILES: Cc1cc([C@@H](C)Nc2ccccc2C(=O)O)c2nc(-c3cc4c(N)nn(C)c4cc3C)c(C)c(=O)n2c1] | 732 |
| 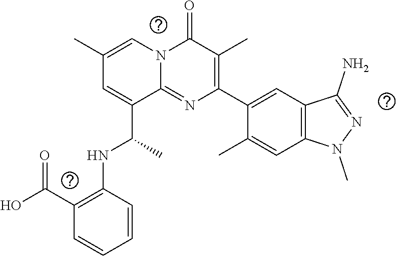 [SMILES: Cc1cc([C@H](C)Nc2ccccc2C(=O)O)c2nc(-c3cc4c(N)nn(C)c4cc3C)c(C)c(=O)n2c1] | 733 |
| 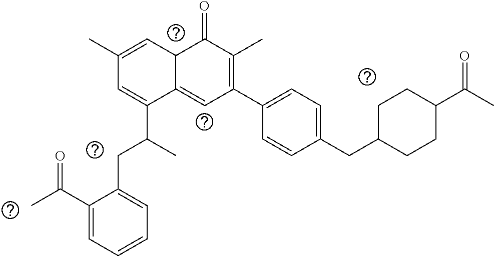 [SMILES: CC(=O)c1ccccc1CC(C)C1=CC(C)=CC2C(=O)C(C)=C(c3ccc(CC4CCC(C(C)=O)CC4)cc3)C=C12] | 734 |
| 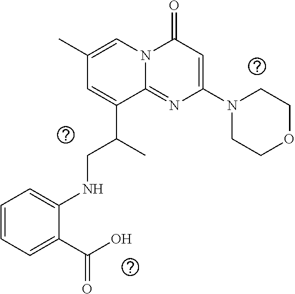 [SMILES: Cc1cc(C(C)CNc2ccccc2C(=O)O)c2nc(N3CCOCC3)cc(=O)n2c1] | 735 |
| 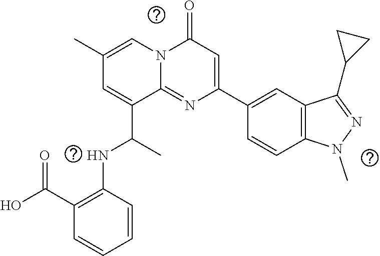 [SMILES: Cc1cc(C(C)Nc2ccccc2C(=O)O)c2nc(-c3ccc4c(c3)c(C3CC3)nn4C)cc(=O)n2c1] | 736 |
| 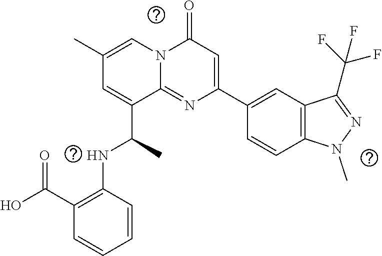 [SMILES: Cc1cc([C@@H](C)Nc2ccccc2C(=O)O)c2nc(-c3ccc4c(c3)c(C(F)(F)F)nn4C)cc(=O)n2c1] | 737 |
| 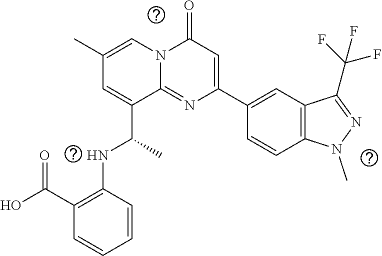 [SMILES: Cc1cc([C@H](C)Nc2ccccc2C(=O)O)c2nc(-c3ccc4c(c3)c(C(F)(F)F)nn4C)cc(=O)n2c1] | 738 |
| 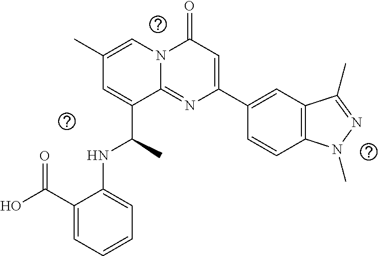 [SMILES: Cc1cc([C@@H](C)Nc2ccccc2C(=O)O)c2nc(-c3ccc4c(c3)c(C)nn4C)cc(=O)n2c1] | 739 |
| 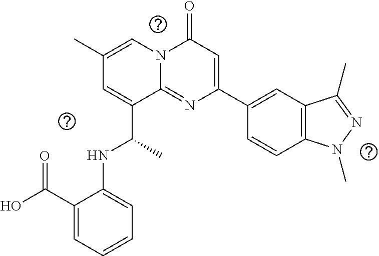 [SMILES: Cc1cc([C@H](C)Nc2ccccc2C(=O)O)c2nc(-c3ccc4c(c3)c(C)nn4C)cc(=O)n2c1] | 740 |
| 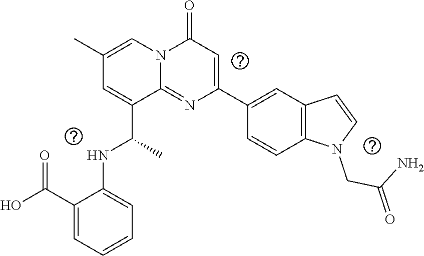 [SMILES: Cc1cc([C@H](C)Nc2ccccc2C(=O)O)c2nc(-c3ccc4c(ccn4CC(N)=O)c3)cc(=O)n2c1] | 741 |
| 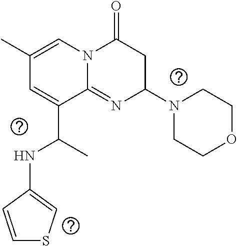 [SMILES: CC1=CN2C(=O)CC(N3CCOCC3)N=C2C(C(C)Nc2ccsc2)=C1] | 742 |
| 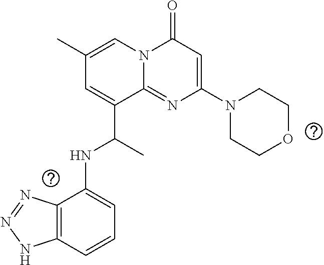 [SMILES: Cc1cc(C(C)Nc2cccc3[nH]nnc23)c2nc(N3CCOCC3)cc(=O)n2c1] | 743 |
| 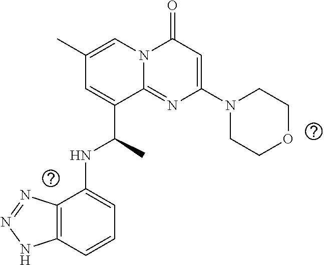 [SMILES: Cc1cc([C@@H](C)Nc2cccc3[nH]nnc23)c2nc(N3CCOCC3)cc(=O)n2c1] | 744 |
| 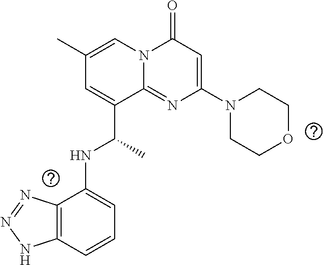 [SMILES: Cc1cc([C@H](C)Nc2cccc3[nH]nnc23)c2nc(N3CCOCC3)cc(=O)n2c1] | 745 |
| 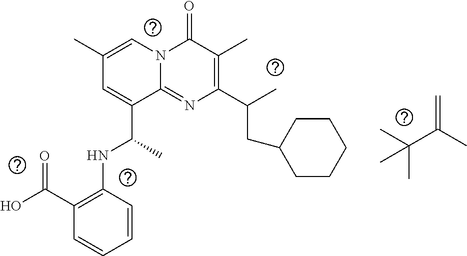 | 746 |
| 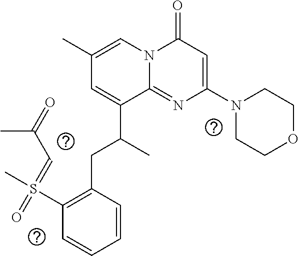 [SMILES: CC(=O)C=S(C)(=O)c1ccccc1CC(C)c1cc(C)cn2c(=O)cc(N3CCOCC3)nc12] | 747 |
| 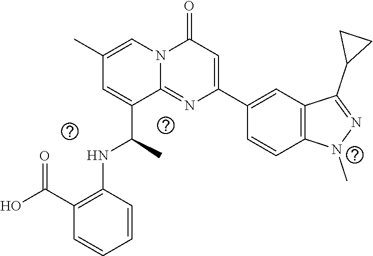 [SMILES: Cc1cc([C@@H](C)Nc2ccccc2C(=O)O)c2nc(-c3ccc4c(c3)c(C3CC3)nn4C)cc(=O)n2c1] | 748 |
| 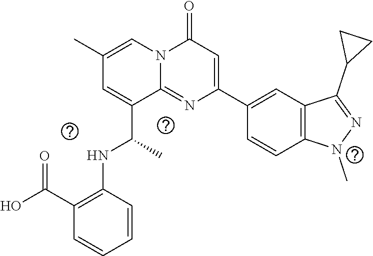 [SMILES: Cc1cc([C@H](C)Nc2ccccc2C(=O)O)c2nc(-c3ccc4c(c3)c(C3CC3)nn4C)cc(=O)n2c1] | 749 |
| 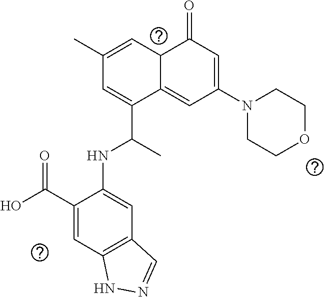 [SMILES: CC1=CC2C(=O)C=C(N3CCOCC3)C=C2C(C(C)Nc2cc3cn[nH]c3cc2C(=O)O)=C1] | 750 |
| 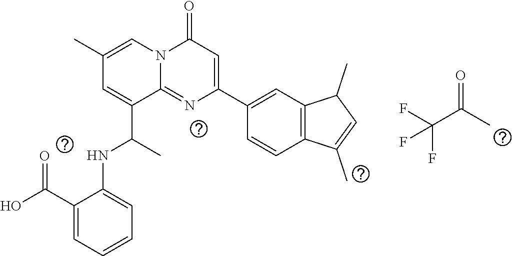 | 751 |
| 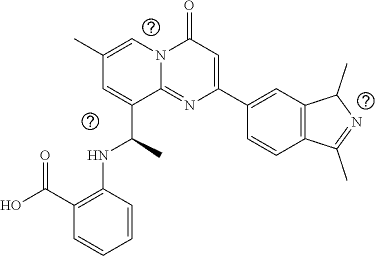 [SMILES: CC1=NC(C)c2cc(-c3cc(=O)n4cc(C)cc([C@@H](C)Nc5ccccc5C(=O)O)c4n3)ccc21] | 752 |
| 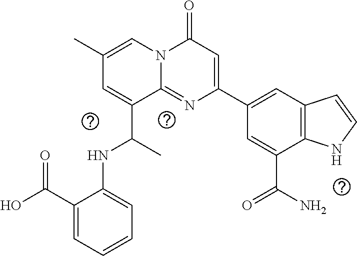 [SMILES: Cc1cc(C(C)Nc2ccccc2C(=O)O)c2nc(-c3cc(C(N)=O)c4[nH]ccc4c3)cc(=O)n2c1] | 753 |
| 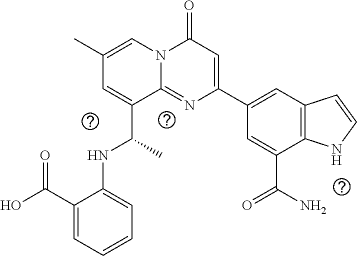 [SMILES: Cc1cc([C@H](C)Nc2ccccc2C(=O)O)c2nc(-c3cc(C(N)=O)c4[nH]ccc4c3)cc(=O)n2c1] | 754 |
| 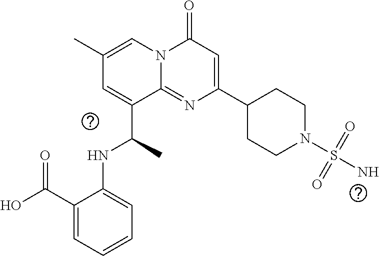 [SMILES: Cc1cc([C@@H](C)Nc2ccccc2C(=O)O)c2nc(C3CCN(S(N)(=O)=O)CC3)cc(=O)n2c1] | 755 |
| 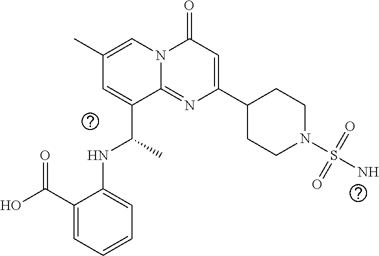 [SMILES: Cc1cc([C@H](C)Nc2ccccc2C(=O)O)c2nc(C3CCN(S(N)(=O)=O)CC3)cc(=O)n2c1] | 756 |
| 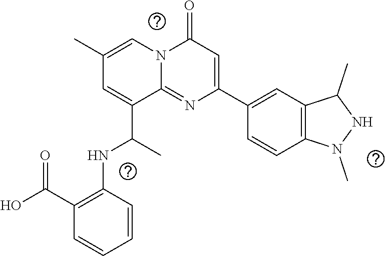 [SMILES: Cc1cc(C(C)Nc2ccccc2C(=O)O)c2nc(-c3ccc4c(c3)C(C)NN4C)cc(=O)n2c1] | 757 |
| 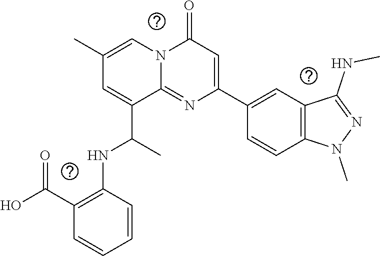 [SMILES: CNc1nn(C)c2ccc(-c3cc(=O)n4cc(C)cc(C(C)Nc5ccccc5C(=O)O)c4n3)cc12] | 758 |
| 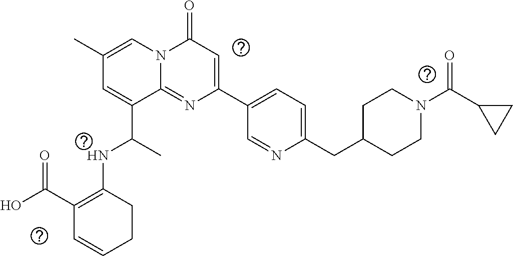 [SMILES: Cc1cc(C(C)NC2=C(C(=O)O)C=CCC2)c2nc(-c3ccc(CC4CCN(C(=O)C5CC5)CC4)nc3)cc(=O)n2c1] | 759 |
| 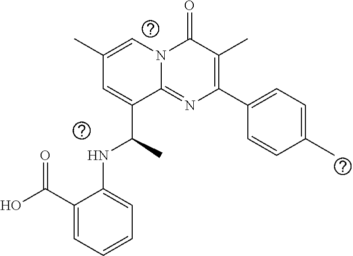 [SMILES: Cc1ccc(-c2nc3c([C@@H](C)Nc4ccccc4C(=O)O)cc(C)cn3c(=O)c2C)cc1] | 760 |
| 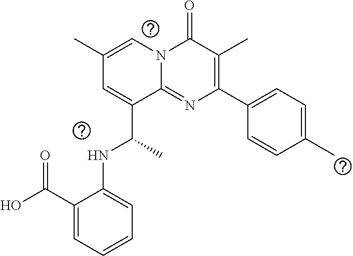 [SMILES: Cc1ccc(-c2nc3c([C@H](C)Nc4ccccc4C(=O)O)cc(C)cn3c(=O)c2C)cc1] | 761 |
| 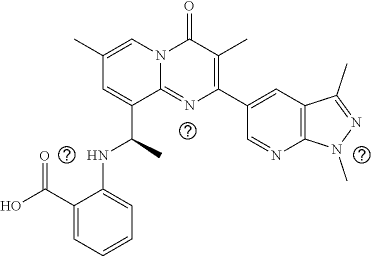 [SMILES: Cc1cc([C@@H](C)Nc2ccccc2C(=O)O)c2nc(-c3cnc4c(c3)c(C)nn4C)c(C)c(=O)n2c1] | 762 |
| 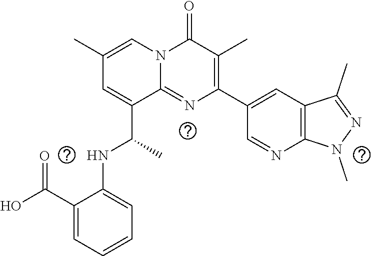 [SMILES: Cc1cc([C@H](C)Nc2ccccc2C(=O)O)c2nc(-c3cnc4c(c3)c(C)nn4C)c(C)c(=O)n2c1] | 763 |
| 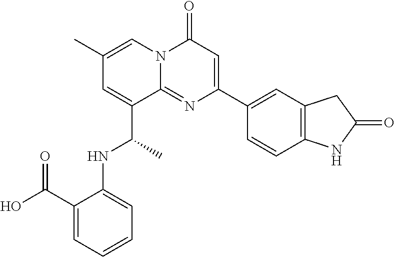 [SMILES: Cc1cc([C@H](C)Nc2ccccc2C(=O)O)c2nc(-c3ccc4c(c3)CC(=O)N4)cc(=O)n2c1] | 764 |
| 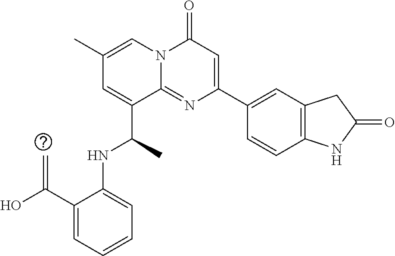 [SMILES: C=C(O)c1ccccc1N[C@H](C)c1cc(C)cn2c(=O)cc(-c3ccc4c(c3)CC(=O)N4)nc12] | 765 |
| 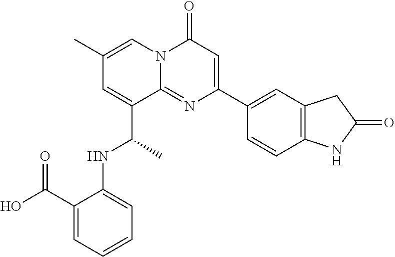 [SMILES: Cc1cc([C@H](C)Nc2ccccc2C(=O)O)c2nc(-c3ccc4c(c3)CC(=O)N4)cc(=O)n2c1] | 766 |
| 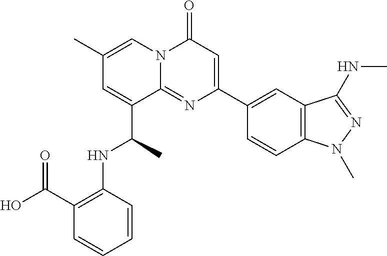 [SMILES: CNc1nn(C)c2ccc(-c3cc(=O)n4cc(C)cc([C@@H](C)Nc5ccccc5C(=O)O)c4n3)cc12] | 767 |
| 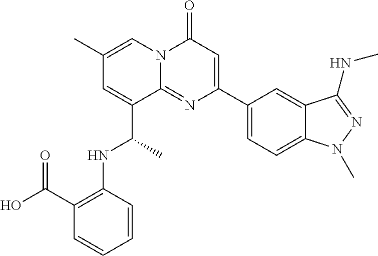 [SMILES: CNc1nn(C)c2ccc(-c3cc(=O)n4cc(C)cc([C@H](C)Nc5ccccc5C(=O)O)c4n3)cc12] | 768 |
| 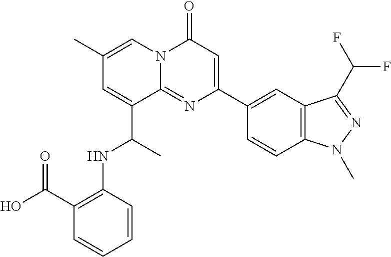 [SMILES: Cc1cc(C(C)Nc2ccccc2C(=O)O)c2nc(-c3ccc4c(c3)c(C(F)F)nn4C)cc(=O)n2c1] | 769 |
| 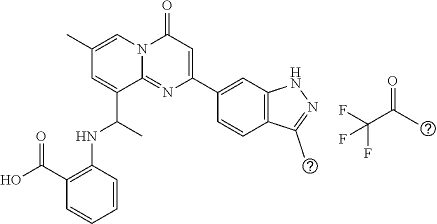 | 770 |
| 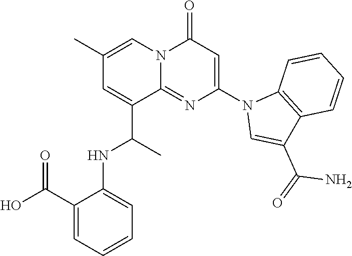 [SMILES: Cc1cc(C(C)Nc2ccccc2C(=O)O)c2nc(-n3cc(C(N)=O)c4ccccc43)cc(=O)n2c1] | 771 |
| 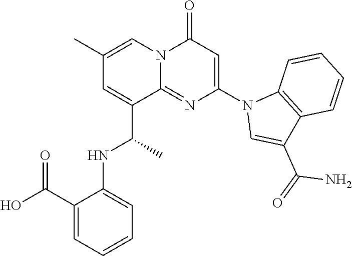 [SMILES: Cc1cc([C@H](C)Nc2ccccc2C(=O)O)c2nc(-n3cc(C(N)=O)c4ccccc43)cc(=O)n2c1] | 772 |
| 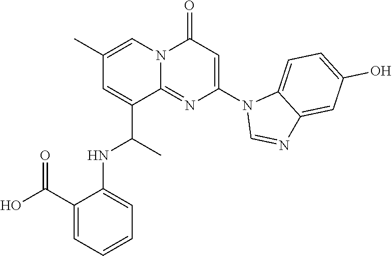 [SMILES: Cc1cc(C(C)Nc2ccccc2C(=O)O)c2nc(-n3cnc4cc(O)ccc43)cc(=O)n2c1] | 773 |
| 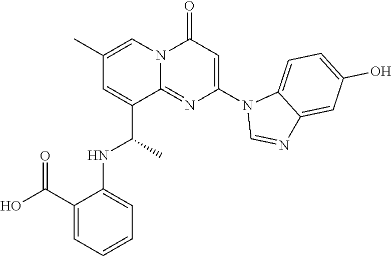 [SMILES: Cc1cc([C@H](C)Nc2ccccc2C(=O)O)c2nc(-n3cnc4cc(O)ccc43)cc(=O)n2c1] | 774 |
| 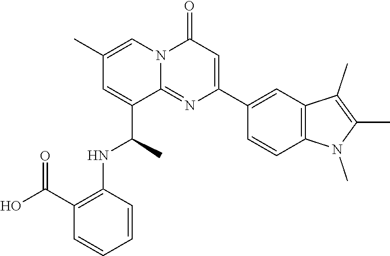 [SMILES: Cc1cc([C@@H](C)Nc2ccccc2C(=O)O)c2nc(-c3ccc4c(c3)c(C)c(C)n4C)cc(=O)n2c1] | 775 |
| 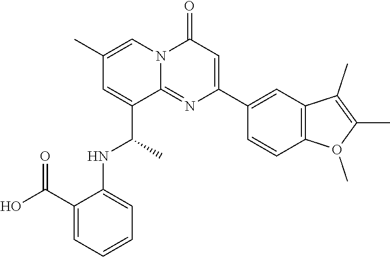 | 776 |
| 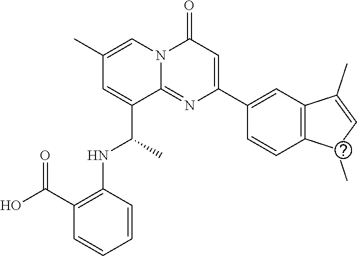 [SMILES: CC1=CC(C)c2ccc(-c3cc(=O)n4cc(C)cc([C@H](C)Nc5ccccc5C(=O)O)c4n3)cc21] | 777 |
| 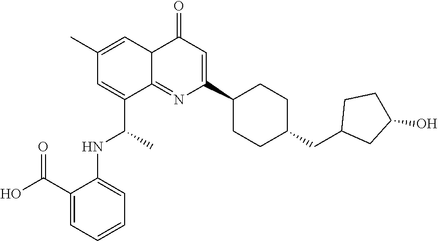 [SMILES: CC1=CC2C(=O)C=C([C@H]3CC[C@H](CC4CC[C@H](O)C4)CC3)N=C2C([C@H](C)Nc2ccccc2C(=O)O)=C1] | 778 |
| 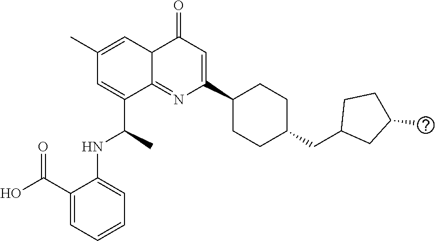 [SMILES: CC1=CC2C(=O)C=C([C@H]3CC[C@H](CC4CC[C@H](C)C4)CC3)N=C2C([C@@H](C)Nc2ccccc2C(=O)O)=C1] | 779 |
| 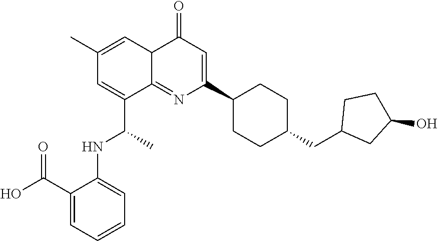 [SMILES: CC1=CC2C(=O)C=C([C@H]3CC[C@H](CC4CC[C@@H](O)C4)CC3)N=C2C([C@H](C)Nc2ccccc2C(=O)O)=C1] | 780 |
| 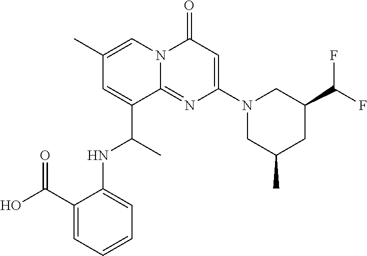 [SMILES: Cc1cc(C(C)Nc2ccccc2C(=O)O)c2nc(N3C[C@H](C)C[C@H](C(F)F)C3)cc(=O)n2c1] | 781 |
| 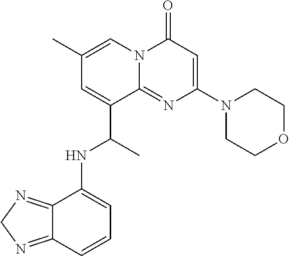 [SMILES: Cc1cc(C(C)Nc2cccc3c2=NCN=3)c2nc(N3CCOCC3)cc(=O)n2c1] | 782 |
| 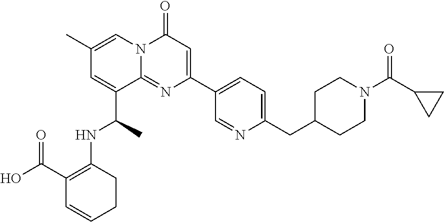 [SMILES: Cc1cc([C@@H](C)NC2=C(C(=O)O)C=CCC2)c2nc(-c3ccc(CC4CCN(C(=O)C5CC5)CC4)nc3)cc(=O)n2c1] | 783 |
| 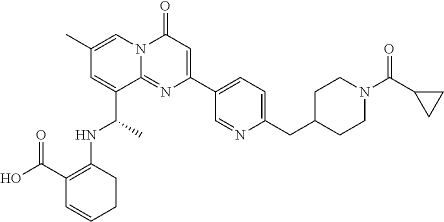 [SMILES: Cc1cc([C@H](C)NC2=C(C(=O)O)C=CCC2)c2nc(-c3ccc(CC4CCN(C(=O)C5CC5)CC4)nc3)cc(=O)n2c1] | 784 |
| 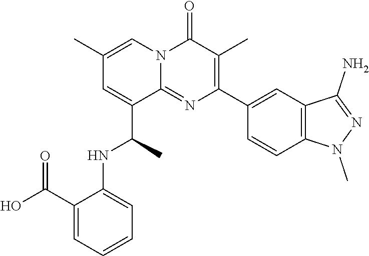 [SMILES: Cc1cc([C@@H](C)Nc2ccccc2C(=O)O)c2nc(-c3ccc4c(c3)c(N)nn4C)c(C)c(=O)n2c1] | 785 |
| 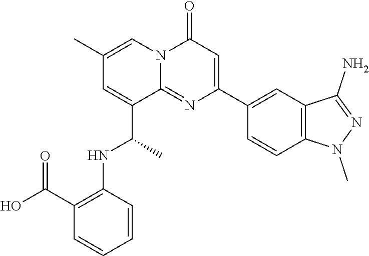 [SMILES: Cc1cc([C@H](C)Nc2ccccc2C(=O)O)c2nc(-c3ccc4c(c3)c(N)nn4C)cc(=O)n2c1] | 786 |
| 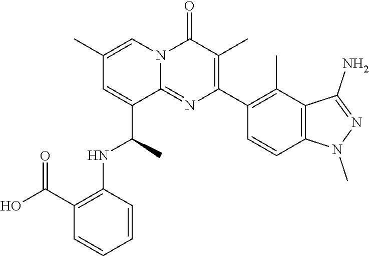 [SMILES: Cc1cc([C@@H](C)Nc2ccccc2C(=O)O)c2nc(-c3ccc4c(c(N)nn4C)c3C)c(C)c(=O)n2c1] | 787 |
| 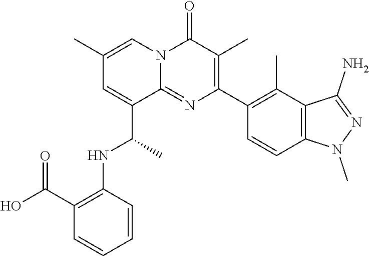 [SMILES: Cc1cc([C@H](C)Nc2ccccc2C(=O)O)c2nc(-c3ccc4c(c(N)nn4C)c3C)c(C)c(=O)n2c1] | 788 |
| 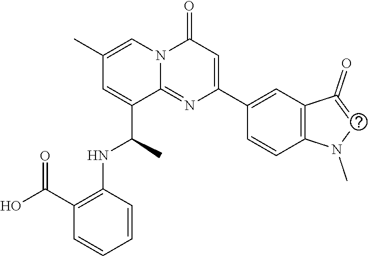 | 789 |
| 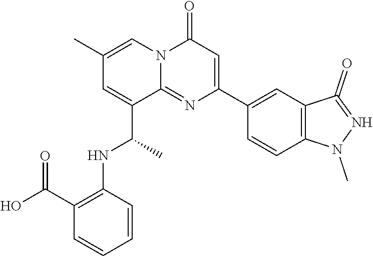 | 790 |
| 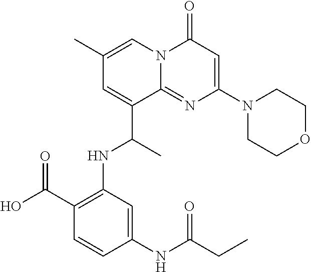 [SMILES: CCC(=O)Nc1ccc(C(=O)O)c(NC(C)c2cc(C)cn3c(=O)cc(N4CCOCC4)nc23)c1] | 791 |
| 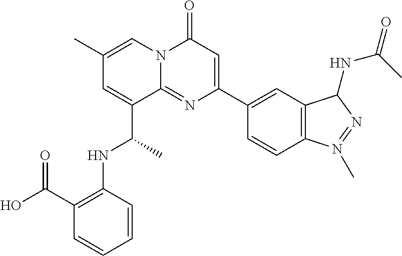 | 792 |
| 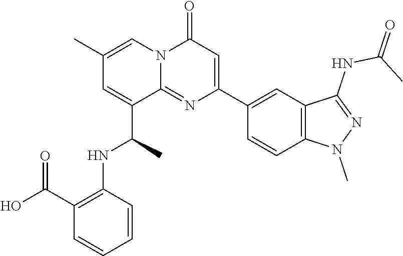 [SMILES: CC(=O)Nc1nn(C)c2ccc(-c3cc(=O)n4cc(C)cc([C@@H](C)Nc5ccccc5C(=O)O)c4n3)cc12] | 793 |
| 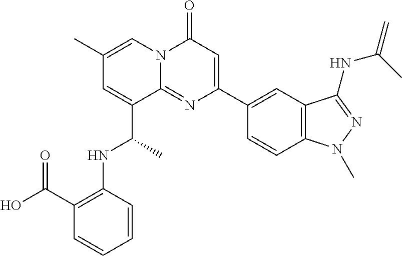 [SMILES: C=C(C)Nc1nn(C)c2ccc(-c3cc(=O)n4cc(C)cc([C@H](C)Nc5ccccc5C(=O)O)c4n3)cc12] | 794 |
| 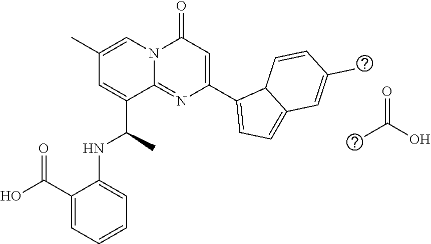 | 795 |
| 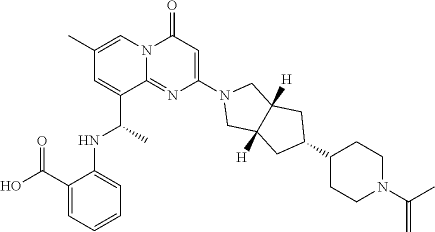 [SMILES: C=C(C)N1CCC([C@@H]2C[C@@H]3CN(c4cc(=O)n5cc(C)cc([C@H](C)Nc6ccccc6C(=O)O)c5n4)C[C@@H]3C2)CC1] | 796 |
| 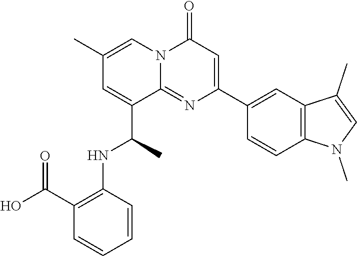 [SMILES: Cc1cc([C@@H](C)Nc2ccccc2C(=O)O)c2nc(-c3ccc4c(c3)c(C)cn4C)cc(=O)n2c1] | 797 |
| 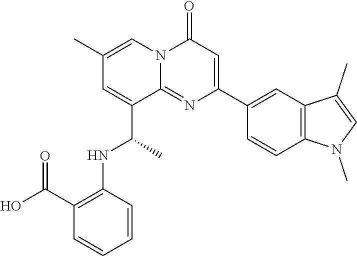 [SMILES: Cc1cc([C@H](C)Nc2ccccc2C(=O)O)c2nc(-c3ccc4c(c3)c(C)cn4C)cc(=O)n2c1] | 798 |
| 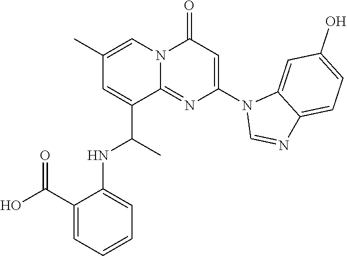 [SMILES: Cc1cc(C(C)Nc2ccccc2C(=O)O)c2nc(-n3cnc4ccc(O)cc43)cc(=O)n2c1] | 799 |
| 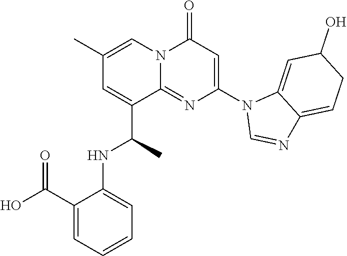 [SMILES: Cc1cc([C@@H](C)Nc2ccccc2C(=O)O)c2nc(-n3cnc4c3=CC(O)CC=4)cc(=O)n2c1] | 800 |
| 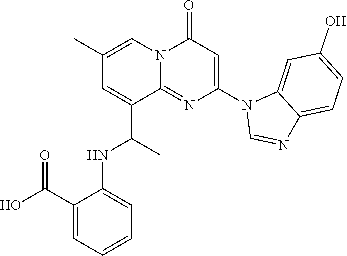 [SMILES: Cc1cc(C(C)Nc2ccccc2C(=O)O)c2nc(-n3cnc4ccc(O)cc43)cc(=O)n2c1] | 801 |
| 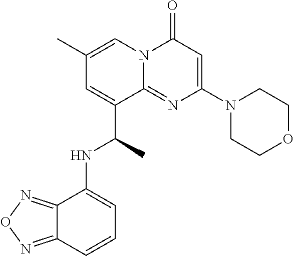 [SMILES: Cc1cc([C@@H](C)Nc2cccc3nonc23)c2nc(N3CCOCC3)cc(=O)n2c1] | 802 |
| 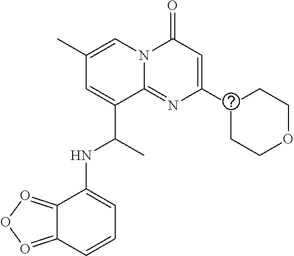 | 803 |
| 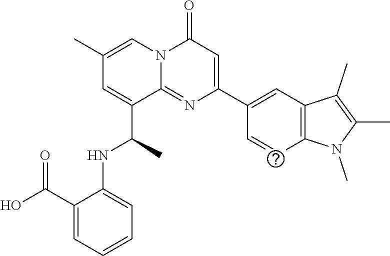 [SMILES: Cc1cc([C@@H](C)Nc2ccccc2C(=O)O)c2nc(-c3ccc4c(c3)c(C)c(C)n4C)cc(=O)n2c1] | 804 |
| 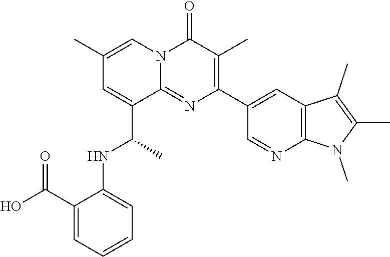 [SMILES: Cc1cc([C@H](C)Nc2ccccc2C(=O)O)c2nc(-c3cnc4c(c3)c(C)c(C)n4C)c(C)c(=O)n2c1] | 805 |
| 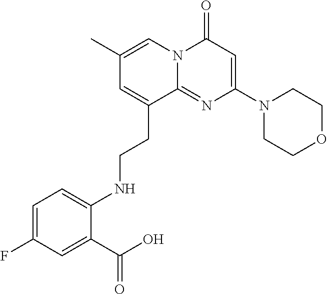 [SMILES: Cc1cc(CCNc2ccc(F)cc2C(=O)O)c2nc(N3CCOCC3)cc(=O)n2c1] | 806 |
| 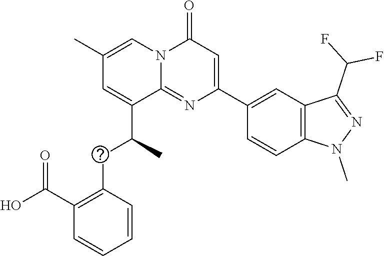 [SMILES: Cc1cc([C@@H](C)Cc2ccccc2C(=O)O)c2nc(-c3ccc4c(c3)c(C(F)F)nn4C)cc(=O)n2c1] | 807 |
| 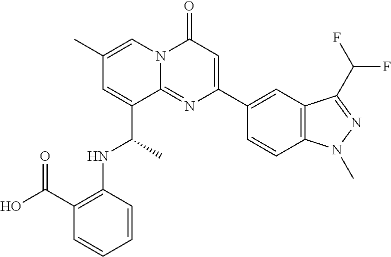 [SMILES: Cc1cc([C@H](C)Nc2ccccc2C(=O)O)c2nc(-c3ccc4c(c3)c(C(F)F)nn4C)cc(=O)n2c1] | 808 |
| 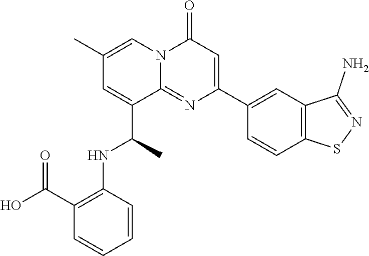 [SMILES: Cc1cc([C@@H](C)Nc2ccccc2C(=O)O)c2nc(-c3ccc4snc(N)c4c3)cc(=O)n2c1] | 809 |
| 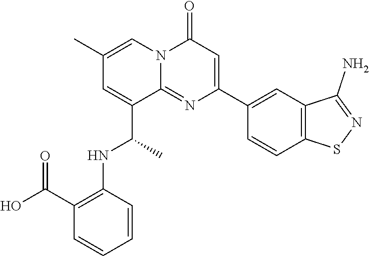 [SMILES: Cc1cc([C@H](C)Nc2ccccc2C(=O)O)c2nc(-c3ccc4snc(N)c4c3)cc(=O)n2c1] | 810 |
| 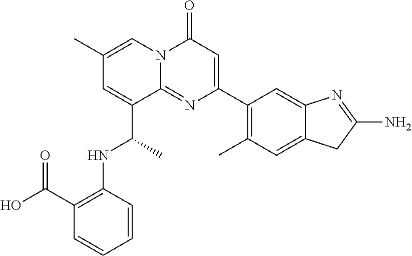 [SMILES: Cc1cc([C@H](C)Nc2ccccc2C(=O)O)c2nc(-c3cc4c(cc3C)CC(N)=N4)cc(=O)n2c1] | 811 |
| 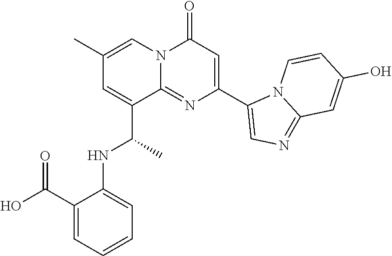 [SMILES: Cc1cc([C@H](C)Nc2ccccc2C(=O)O)c2nc(-c3cnc4cc(O)ccn34)cc(=O)n2c1] | 812 |
| 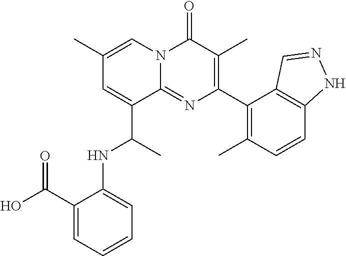 [SMILES: Cc1cc(C(C)Nc2ccccc2C(=O)O)c2nc(-c3c(C)ccc4[nH]ncc34)c(C)c(=O)n2c1] | 813 |
| 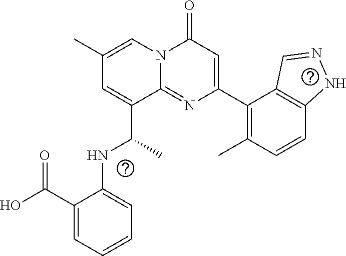 [SMILES: Cc1cc([C@H](C)Nc2ccccc2C(=O)O)c2nc(-c3c(C)ccc4[nH]ncc34)cc(=O)n2c1] | 814 |
| 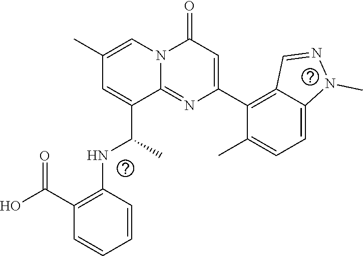 [SMILES: Cc1cc([C@H](C)Nc2ccccc2C(=O)O)c2nc(-c3c(C)ccc4c3cnn4C)cc(=O)n2c1] | 815 |
| 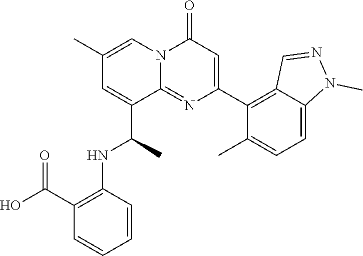 [SMILES: Cc1cc([C@@H](C)Nc2ccccc2C(=O)O)c2nc(-c3c(C)ccc4c3cnn4C)cc(=O)n2c1] | 816 |
| 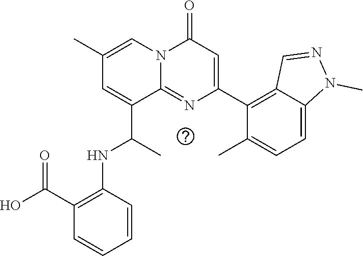 [SMILES: Cc1cc(C(C)Nc2ccccc2C(=O)O)c2nc(-c3c(C)ccc4c3cnn4C)cc(=O)n2c1] | 817 |
| 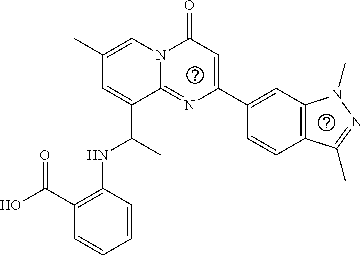 [SMILES: Cc1cc(C(C)Nc2ccccc2C(=O)O)c2nc(-c3ccc4c(C)nn(C)c4c3)cc(=O)n2c1] | 818 |
| 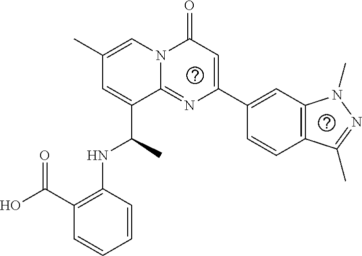 [SMILES: Cc1cc([C@@H](C)Nc2ccccc2C(=O)O)c2nc(-c3ccc4c(C)nn(C)c4c3)cc(=O)n2c1] | 819 |
| 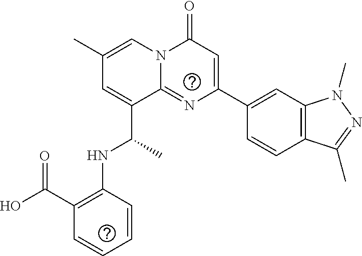 [SMILES: Cc1cc([C@H](C)Nc2ccccc2C(=O)O)c2nc(-c3ccc4c(C)nn(C)c4c3)cc(=O)n2c1] | 820 |
| 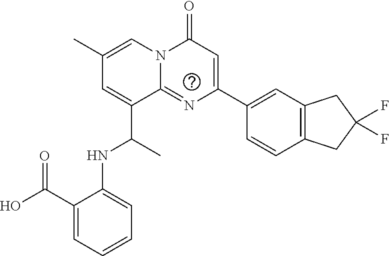 [SMILES: Cc1cc(C(C)Nc2ccccc2C(=O)O)c2nc(-c3ccc4c(c3)CC(F)(F)C4)cc(=O)n2c1] | 821 |
| 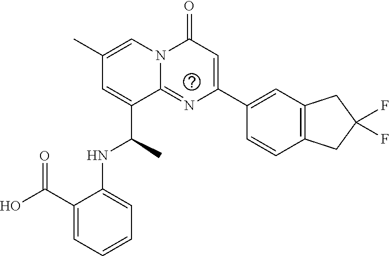 [SMILES: Cc1cc([C@@H](C)Nc2ccccc2C(=O)O)c2nc(-c3ccc4c(c3)CC(F)(F)C4)cc(=O)n2c1] | 822 |
| 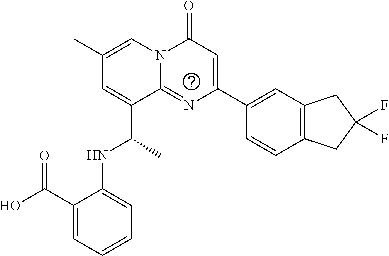 [SMILES: Cc1cc([C@H](C)Nc2ccccc2C(=O)O)c2nc(-c3ccc4c(c3)CC(F)(F)C4)cc(=O)n2c1] | 823 |
| 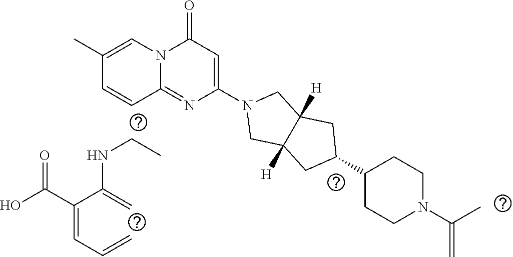 | 824 |
| 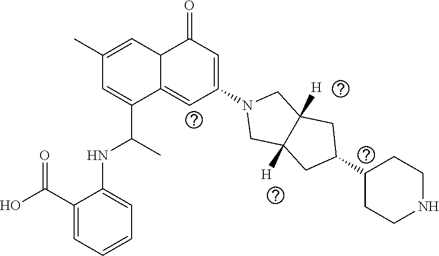 | 825 |
| 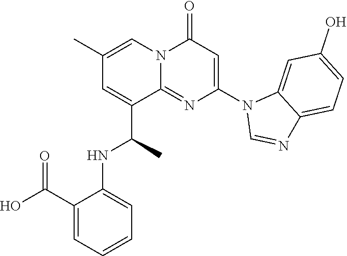 [SMILES: Cc1cc([C@@H](C)Nc2ccccc2C(=O)O)c2nc(-n3cnc4ccc(O)cc43)cc(=O)n2c1] | 826 |
| 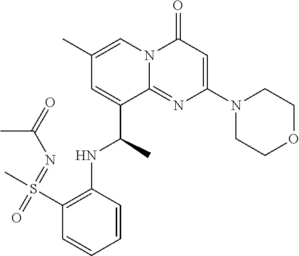 [SMILES: CC(=O)N=S(C)(=O)c1ccccc1N[C@H](C)c1cc(C)cn2c(=O)cc(N3CCOCC3)nc12] | 827 |
| 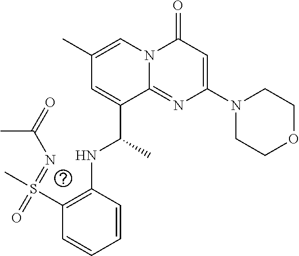 [SMILES: CC(=O)N=S(C)(=O)c1ccccc1N[C@@H](C)c1cc(C)cn2c(=O)cc(N3CCOCC3)nc12] | 828 |
| 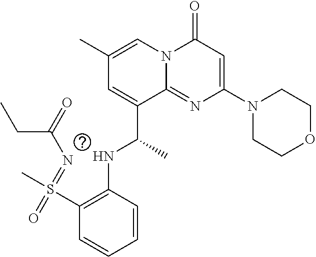 [SMILES: CCC(=O)N=S(C)(=O)c1ccccc1N[C@@H](C)c1cc(C)cn2c(=O)cc(N3CCOCC3)nc12] | 829 |
| 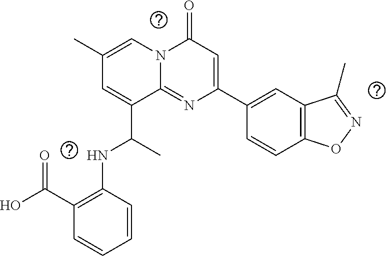 [SMILES: Cc1cc(C(C)Nc2ccccc2C(=O)O)c2nc(-c3ccc4onc(C)c4c3)cc(=O)n2c1] | 830 |
| 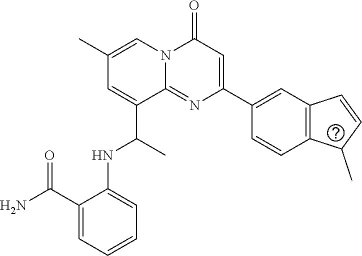 [SMILES: Cc1cc(C(C)Nc2ccccc2C(N)=O)c2nc(-c3ccc4c(c3)C=CC4C)cc(=O)n2c1] | 831 |
| 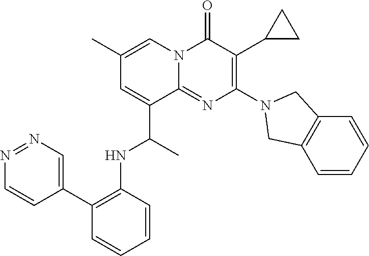 [SMILES: Cc1cc(C(C)Nc2ccccc2-c2ccnnc2)c2nc(N3Cc4ccccc4C3)c(C3CC3)c(=O)n2c1] | 832 |
| 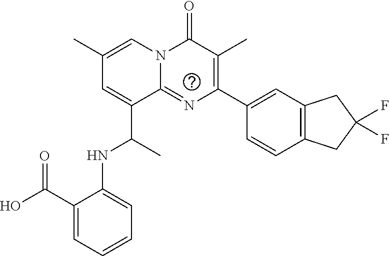 [SMILES: Cc1cc(C(C)Nc2ccccc2C(=O)O)c2nc(-c3ccc4c(c3)CC(F)(F)C4)c(C)c(=O)n2c1] | 833 |
| 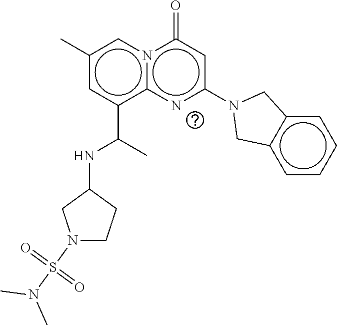 | 834 |
| 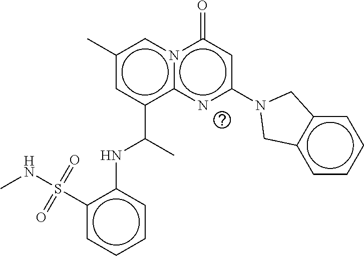 | 835 |
| 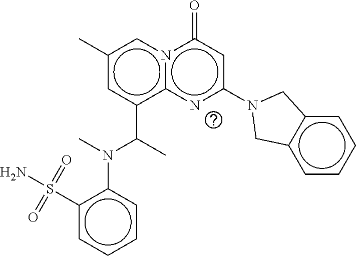 | 836 |
| 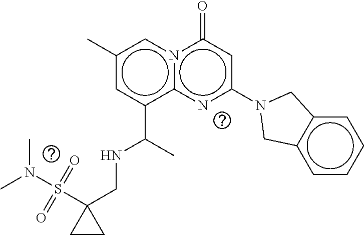 | 837 |
| 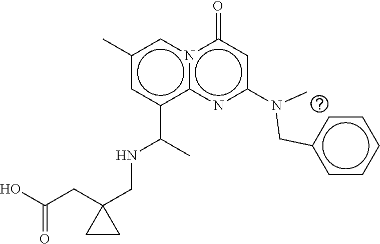 | 838 |
| 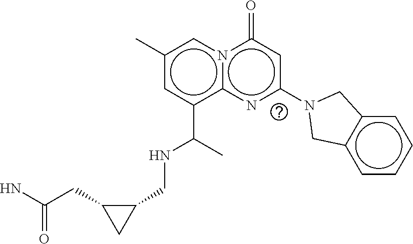 | 839 |
| 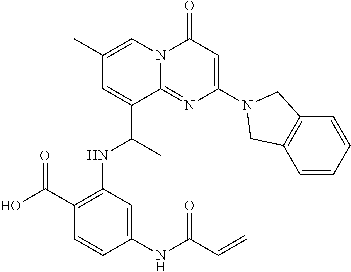 [SMILES: C=CC(=O)Nc1ccc(C(=O)O)c(NC(C)c2cc(C)cn3c(=O)cc(N4Cc5ccccc5C4)nc23)c1] | 840 |
| 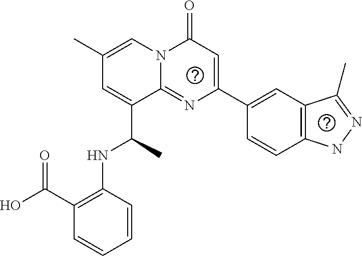 [SMILES: Cc1cc([C@@H](C)Nc2ccccc2C(=O)O)c2nc(-c3ccc4[nH]nc(C)c4c3)cc(=O)n2c1] | 841 |
| 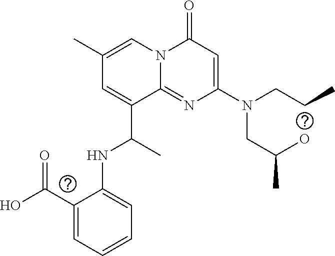 | 842 |
| 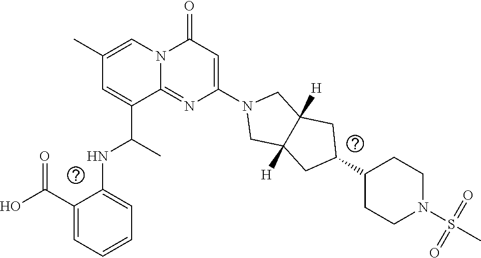 [SMILES: Cc1cc(C(C)Nc2ccccc2C(=O)O)c2nc(N3C[C@H]4C[C@@H](C5CCN(S(C)(=O)=O)CC5)C[C@H]4C3)cc(=O)n2c1] | 843 |
| 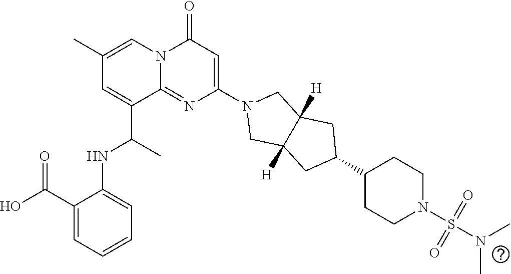 [SMILES: Cc1cc(C(C)Nc2ccccc2C(=O)O)c2nc(N3C[C@H]4C[C@@H](C5CCN(S(=O)(=O)N(C)C)CC5)C[C@H]4C3)cc(=O)n2c1] | 844 |
| 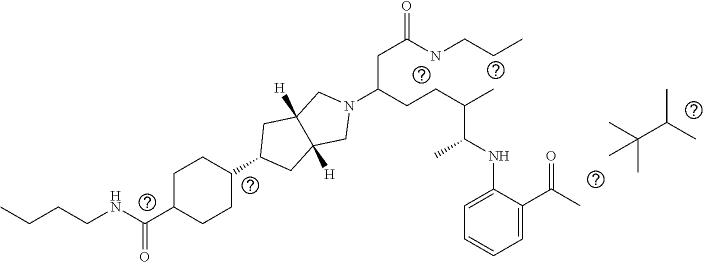 | 845 |
| 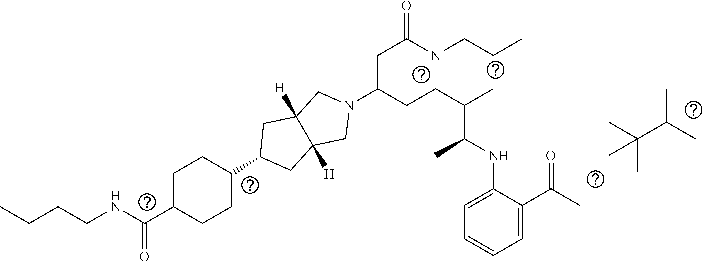 | 846 |
| 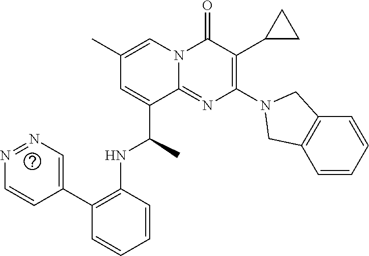 [SMILES: Cc1cc([C@@H](C)Nc2ccccc2-c2ccnnc2)c2nc(N3Cc4ccccc4C3)c(C3CC3)c(=O)n2c1] | 847 |
| 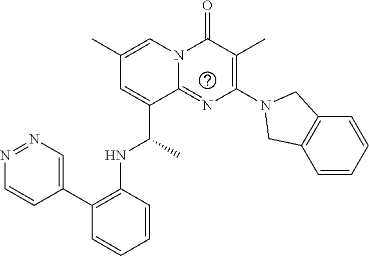 [SMILES: Cc1cc([C@H](C)Nc2ccccc2-c2ccnnc2)c2nc(N3Cc4ccccc4C3)c(C)c(=O)n2c1] | 848 |
| 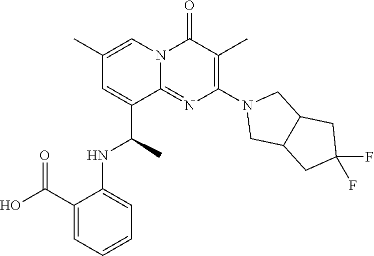 [SMILES: Cc1cc([C@@H](C)Nc2ccccc2C(=O)O)c2nc(N3CC4CC(F)(F)CC4C3)c(C)c(=O)n2c1] | 849 |
| 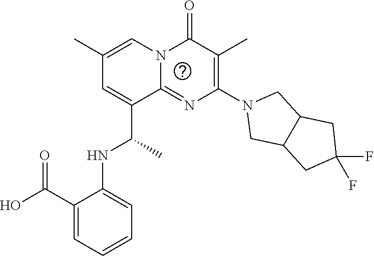 [SMILES: Cc1cc([C@H](C)Nc2ccccc2C(=O)O)c2nc(N3CC4CC(F)(F)CC4C3)c(C)c(=O)n2c1] | 850 |
| 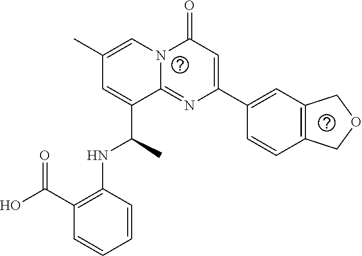 [SMILES: Cc1cc([C@@H](C)Nc2ccccc2C(=O)O)c2nc(-c3ccc4c(c3)COC4)cc(=O)n2c1] | 851 |
| 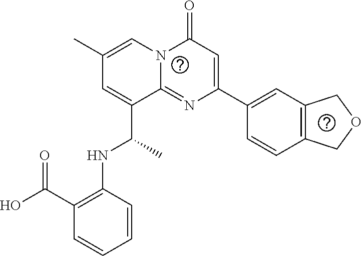 [SMILES: Cc1cc([C@H](C)Nc2ccccc2C(=O)O)c2nc(-c3ccc4c(c3)COC4)cc(=O)n2c1] | 852 |
| 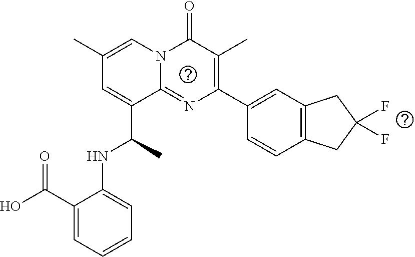 [SMILES: Cc1cc([C@@H](C)Nc2ccccc2C(=O)O)c2nc(-c3ccc4c(c3)CC(F)(F)C4)c(C)c(=O)n2c1] | 853 |
| 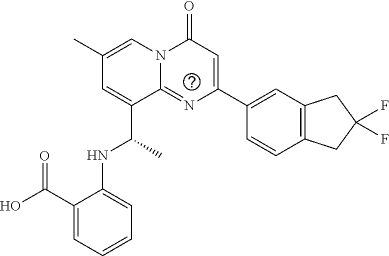 [SMILES: Cc1cc([C@H](C)Nc2ccccc2C(=O)O)c2nc(-c3ccc4c(c3)CC(F)(F)C4)cc(=O)n2c1] | 854 |
| 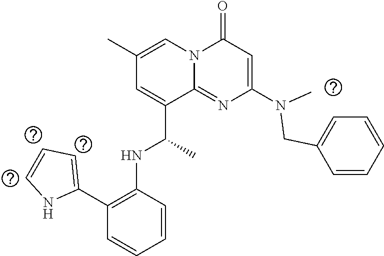 [SMILES: Cc1cc([C@H](C)Nc2ccccc2-c2ccc[nH]2)c2nc(N(C)Cc3ccccc3)cc(=O)n2c1] | 855 |
| 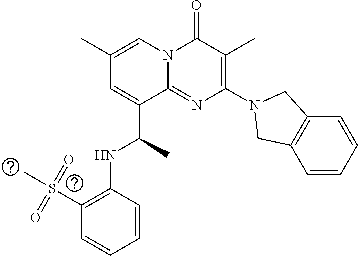 [SMILES: Cc1cc([C@@H](C)Nc2ccccc2S(C)(=O)=O)c2nc(N3Cc4ccccc4C3)c(C)c(=O)n2c1] | 856 |
| 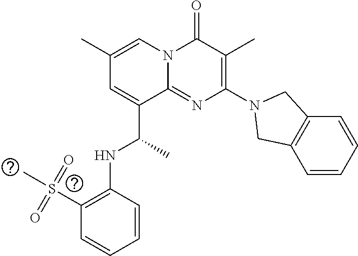 [SMILES: Cc1cc([C@H](C)Nc2ccccc2S(C)(=O)=O)c2nc(N3Cc4ccccc4C3)c(C)c(=O)n2c1] | 857 |
| 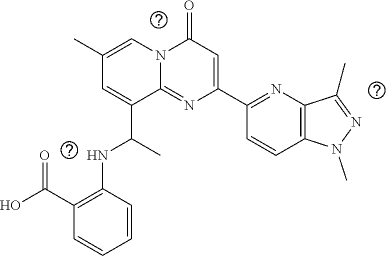 [SMILES: Cc1cc(C(C)Nc2ccccc2C(=O)O)c2nc(-c3ccc4c(n3)c(C)nn4C)cc(=O)n2c1] | 858 |
| 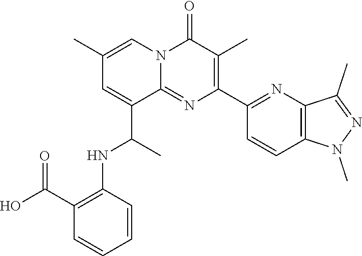 [SMILES: Cc1cc(C(C)Nc2ccccc2C(=O)O)c2nc(-c3ccc4c(n3)c(C)nn4C)c(C)c(=O)n2c1] | 859 |
| 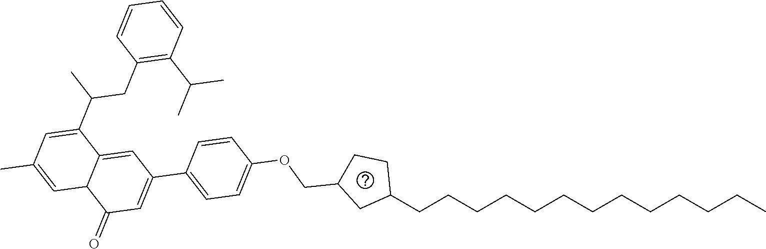 [SMILES: CCCCCCCCCCCCCC1CCC(COc2ccc(C3=CC(=O)C4C=C(C)C=C(C(C)Cc5ccccc5C(C)C)C4=C3)cc2)C1] | 860 |
| 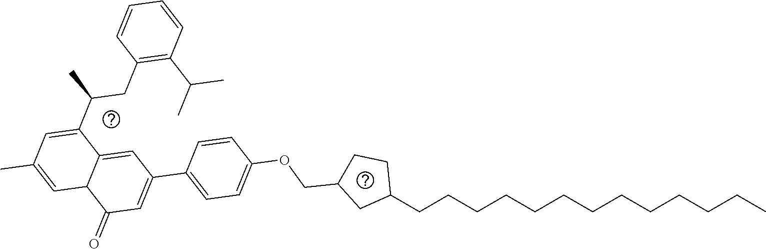 [SMILES: CCCCCCCCCCCCCC1CCC(COc2ccc(C3=CC(=O)C4C=C(C)C=C([C@@H](C)Cc5ccccc5C(C)C)C4=C3)cc2)C1] | 861 |
| 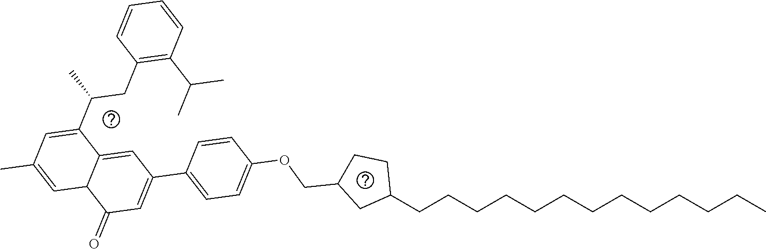 [SMILES: CCCCCCCCCCCCCC1CCC(COc2ccc(C3=CC(=O)C4C=C(C)C=C([C@H](C)Cc5ccccc5C(C)C)C4=C3)cc2)C1] | 862 |
| 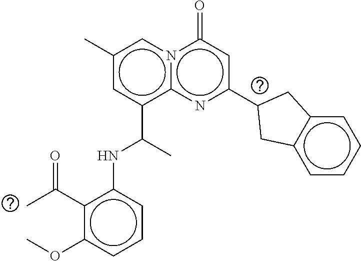 | 863 |
| 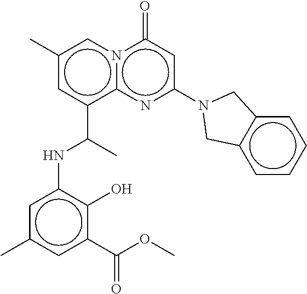 | 864 |
| 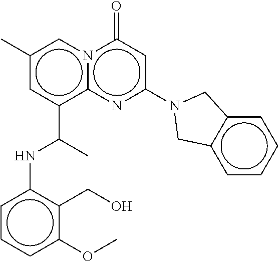 | 865 |
| 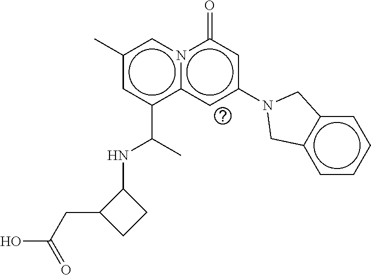 | 866 |
| 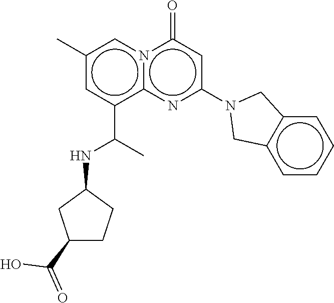 | 867 |
| 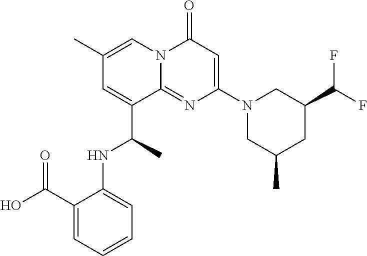 [SMILES: Cc1cc([C@@H](C)Nc2ccccc2C(=O)O)c2nc(N3C[C@H](C)C[C@H](C(F)F)C3)cc(=O)n2c1] | 868 |
| 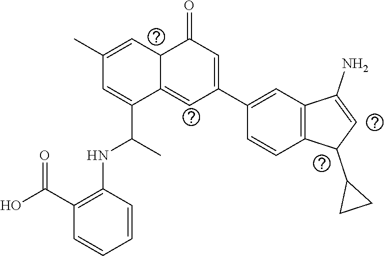 [SMILES: CC1=CC2C(=O)C=C(c3ccc4c(c3)C(N)=CC4C3CC3)C=C2C(C(C)Nc2ccccc2C(=O)O)=C1] | 869 |
| 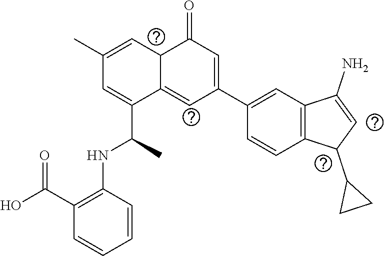 [SMILES: CC1=CC2C(=O)C=C(c3ccc4c(c3)C(N)=CC4C3CC3)C=C2C([C@@H](C)Nc2ccccc2C(=O)O)=C1] | 870 |
| 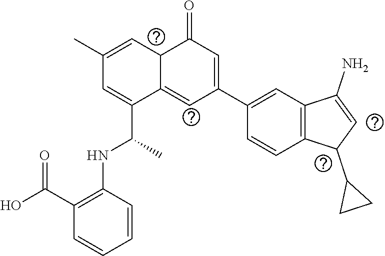 [SMILES: CC1=CC2C(=O)C=C(c3ccc4c(c3)C(N)=CC4C3CC3)C=C2C([C@H](C)Nc2ccccc2C(=O)O)=C1] | 871 |
| 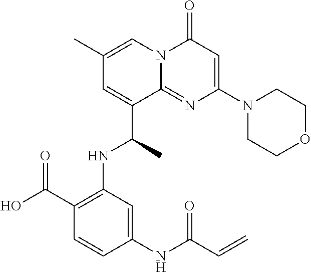 [SMILES: C=CC(=O)Nc1ccc(C(=O)O)c(N[C@H](C)c2cc(C)cn3c(=O)cc(N4CCOCC4)nc23)c1] | 872 |
| 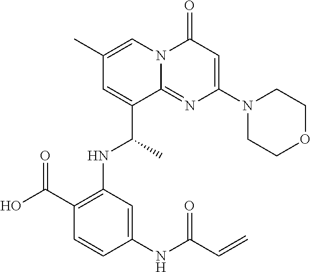 [SMILES: C=CC(=O)Nc1ccc(C(=O)O)c(N[C@@H](C)c2cc(C)cn3c(=O)cc(N4CCOCC4)nc23)c1] | 873 |
| 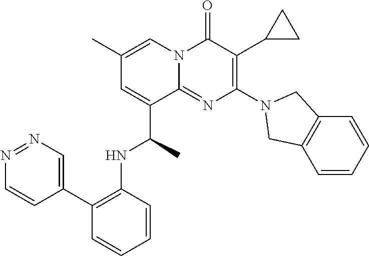 [SMILES: Cc1cc([C@@H](C)Nc2ccccc2-c2ccnnc2)c2nc(N3Cc4ccccc4C3)c(C3CC3)c(=O)n2c1] | 874 |
| 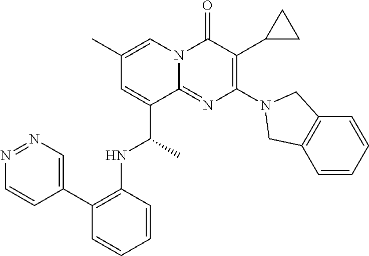 [SMILES: Cc1cc([C@H](C)Nc2ccccc2-c2ccnnc2)c2nc(N3Cc4ccccc4C3)c(C3CC3)c(=O)n2c1] | 875 |
| 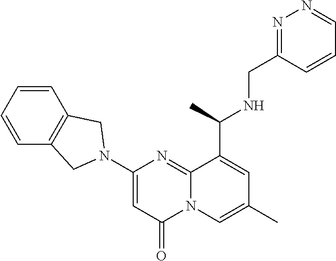 [SMILES: Cc1cc([C@@H](C)NCc2cccnn2)c2nc(N3Cc4ccccc4C3)cc(=O)n2c1] | 876 |
| 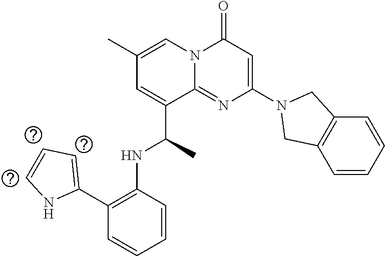 [SMILES: Cc1cc([C@@H](C)Nc2ccccc2-c2ccc[nH]2)c2nc(N3Cc4ccccc4C3)cc(=O)n2c1] | 877 |
| 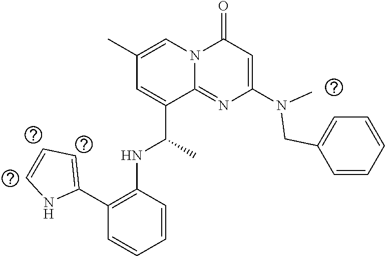 [SMILES: Cc1cc([C@H](C)Nc2ccccc2-c2ccc[nH]2)c2nc(N(C)Cc3ccccc3)cc(=O)n2c1] | 878 |
| 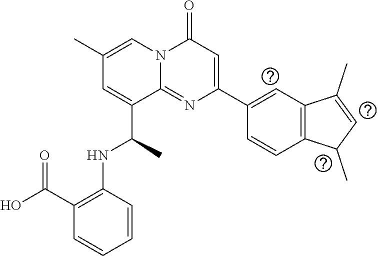 [SMILES: CC1=CC(C)c2ccc(-c3cc(=O)n4cc(C)cc([C@@H](C)Nc5ccccc5C(=O)O)c4n3)cc21] | 879 |
| 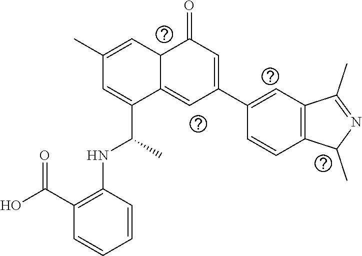 [SMILES: CC1=CC2C(=O)C=C(c3ccc4c(c3)C(C)=NC4C)C=C2C([C@H](C)Nc2ccccc2C(=O)O)=C1] | 880 |
| 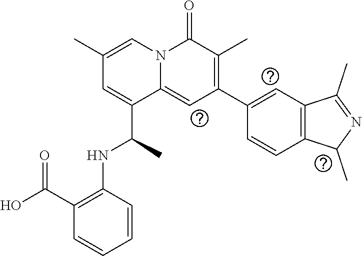 [SMILES: CC1=NC(C)c2ccc(-c3cc4c([C@@H](C)Nc5ccccc5C(=O)O)cc(C)cn4c(=O)c3C)cc21] | 881 |
| 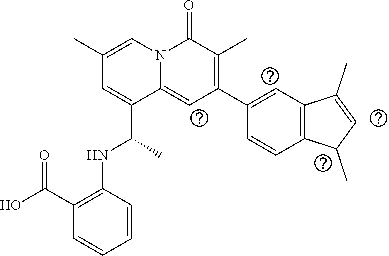 [SMILES: CC1=CC(C)c2ccc(-c3cc4c([C@H](C)Nc5ccccc5C(=O)O)cc(C)cn4c(=O)c3C)cc21] | 882 |
| 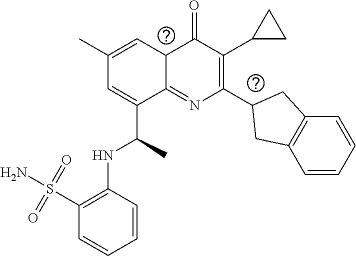 [SMILES: CC1=CC2C(=O)C(C3CC3)=C(C3Cc4ccccc4C3)N=C2C([C@@H](C)Nc2ccccc2S(N)(=O)=O)=C1] | 883 |
| 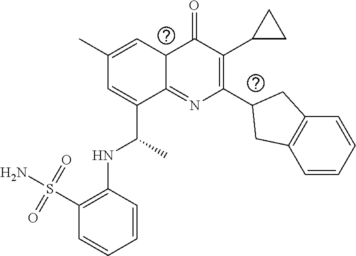 [SMILES: CC1=CC2C(=O)C(C3CC3)=C(C3Cc4ccccc4C3)N=C2C([C@H](C)Nc2ccccc2S(N)(=O)=O)=C1] | 884 |
| 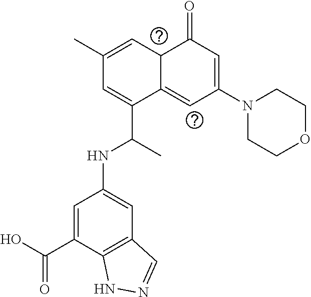 [SMILES: CC1=CC2C(=O)C=C(N3CCOCC3)C=C2C(C(C)Nc2cc(C(=O)O)c3[nH]ncc3c2)=C1] | 885 |
| 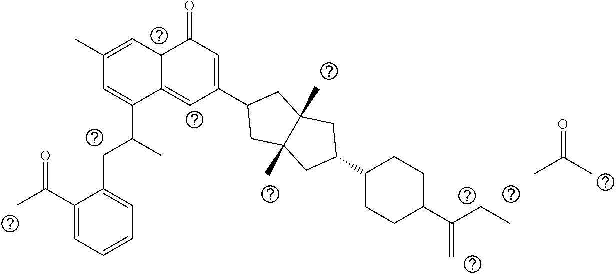 | 886 |
| 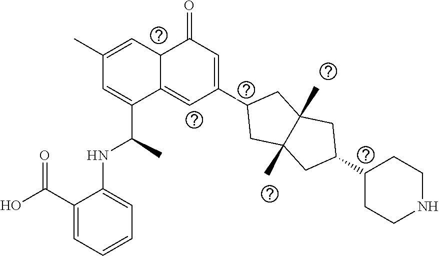 [SMILES: CC1=CC2C(=O)C=C(C3C[C@@]4(C)C[C@@H](C5CCNCC5)C[C@@]4(C)C3)C=C2C([C@@H](C)Nc2ccccc2C(=O)O)=C1] | 887 |
| 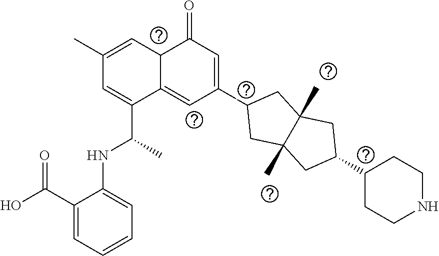 [SMILES: CC1=CC2C(=O)C=C(C3C[C@@]4(C)C[C@@H](C5CCNCC5)C[C@@]4(C)C3)C=C2C([C@H](C)Nc2ccccc2C(=O)O)=C1] | 888 |
| 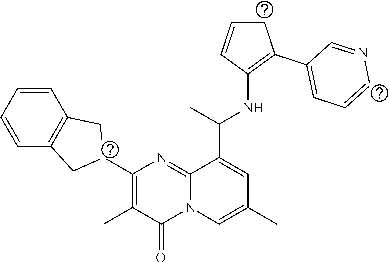 [SMILES: Cc1cc(C(C)NC2=C(c3cccnc3)CC=C2)c2nc(C3Cc4ccccc4C3)c(C)c(=O)n2c1] | 889 |
| 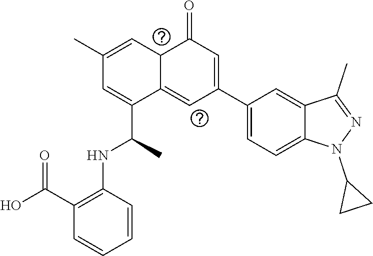 [SMILES: CC1=CC2C(=O)C=C(c3ccc4c(c3)c(C)nn4C3CC3)C=C2C([C@@H](C)Nc2ccccc2C(=O)O)=C1] | 890 |
| 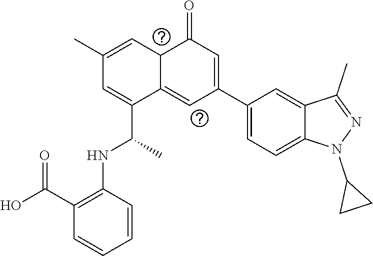 [SMILES: CC1=CC2C(=O)C=C(c3ccc4c(c3)c(C)nn4C3CC3)C=C2C([C@H](C)Nc2ccccc2C(=O)O)=C1] | 891 |
| 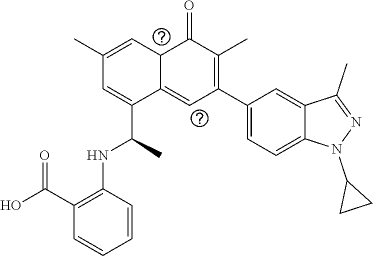 [SMILES: CC1=CC2C(=O)C(C)=C(c3ccc4c(c3)c(C)nn4C3CC3)C=C2C([C@@H](C)Nc2ccccc2C(=O)O)=C1] | 892 |
| 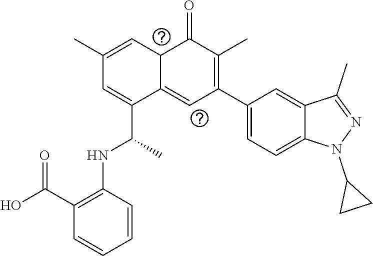 [SMILES: CC1=CC2C(=O)C(C)=C(c3ccc4c(c3)c(C)nn4C3CC3)C=C2C([C@H](C)Nc2ccccc2C(=O)O)=C1] | 893 |
| 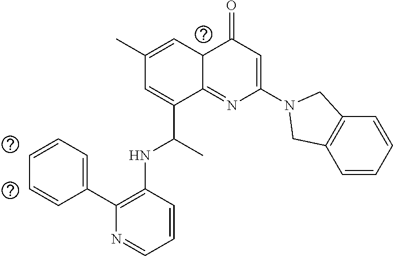 [SMILES: CC1=CC2C(=O)C=C(N3Cc4ccccc4C3)N=C2C(C(C)Nc2cccnc2-c2ccccc2)=C1] | 894 |
| 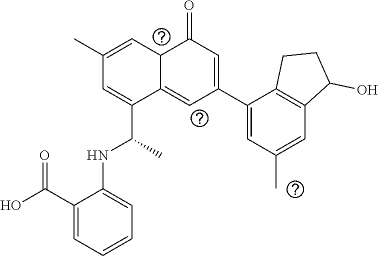 [SMILES: CC1=CC2C(=O)C=C(c3cc(C)cc4c3CCC4O)C=C2C([C@H](C)Nc2ccccc2C(=O)O)=C1] | 895 |
| 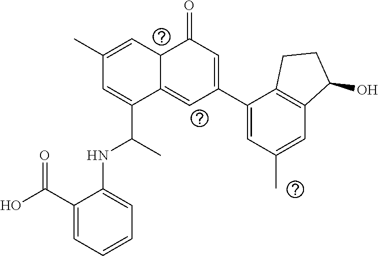 [SMILES: CC1=CC2C(=O)C=C(c3cc(C)cc4c3CC[C@H]4O)C=C2C(C(C)Nc2ccccc2C(=O)O)=C1] | 896 |
| 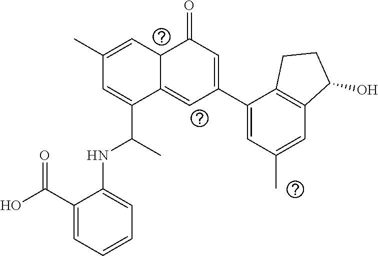 [SMILES: CC1=CC2C(=O)C=C(c3cc(C)cc4c3CC[C@@H]4O)C=C2C(C(C)Nc2ccccc2C(=O)O)=C1] | 897 |
| 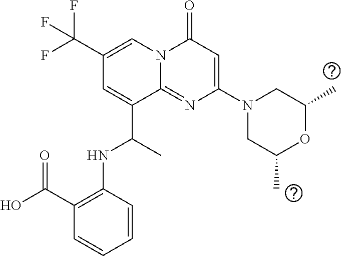 [SMILES: CC(Nc1ccccc1C(=O)O)c1cc(C(F)(F)F)cn2c(=O)cc(N3C[C@@H](C)O[C@@H](C)C3)nc12] | 898 |
| 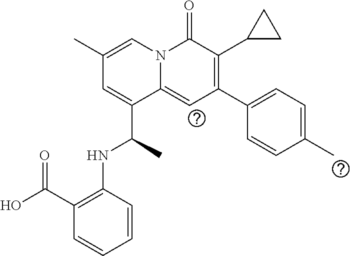 [SMILES: Cc1ccc(-c2cc3c([C@@H](C)Nc4ccccc4C(=O)O)cc(C)cn3c(=O)c2C2CC2)cc1] | 899 |
| 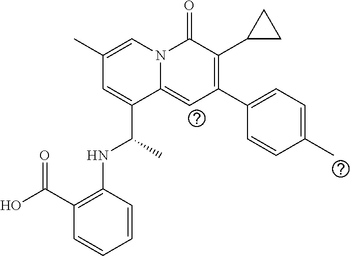 [SMILES: Cc1ccc(-c2cc3c([C@H](C)Nc4ccccc4C(=O)O)cc(C)cn3c(=O)c2C2CC2)cc1] | 900 |
| 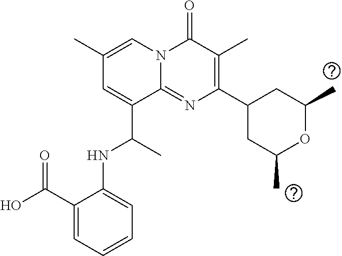 [SMILES: Cc1cc(C(C)Nc2ccccc2C(=O)O)c2nc(C3C[C@@H](C)O[C@@H](C)C3)c(C)c(=O)n2c1] | 901 |
| 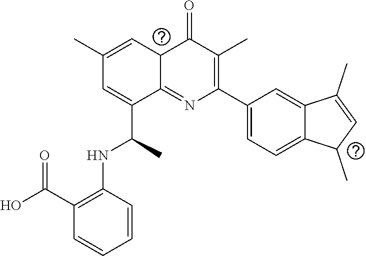 [SMILES: CC1=CC2C(=O)C(C)=C(c3ccc4c(c3)C(C)=CC4C)N=C2C([C@@H](C)Nc2ccccc2C(=O)O)=C1] | 902 |
| 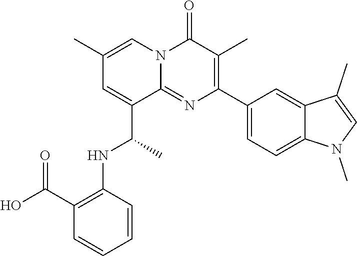 [SMILES: Cc1cc([C@H](C)Nc2ccccc2C(=O)O)c2nc(-c3ccc4c(c3)c(C)cn4C)c(C)c(=O)n2c1] | 903 |
| 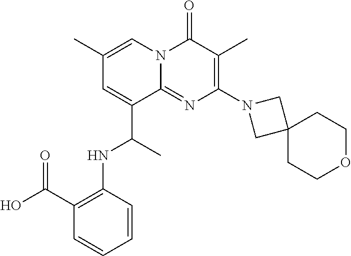 [SMILES: Cc1cc(C(C)Nc2ccccc2C(=O)O)c2nc(N3CC4(CCOCC4)C3)c(C)c(=O)n2c1] | 904 |
| 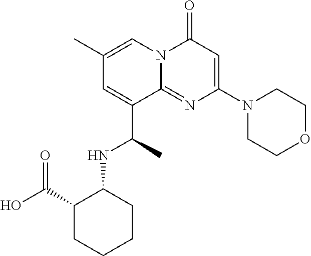 [SMILES: Cc1cc([C@@H](C)N[C@@H]2CCCC[C@@H]2C(=O)O)c2nc(N3CCOCC3)cc(=O)n2c1] | 905 |
| 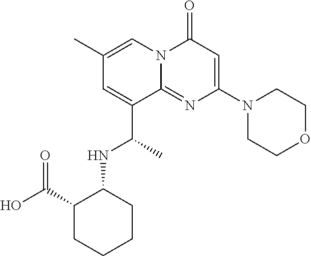 [SMILES: Cc1cc([C@H](C)N[C@@H]2CCCC[C@@H]2C(=O)O)c2nc(N3CCOCC3)cc(=O)n2c1] | 906 |
| 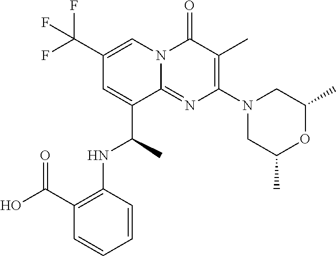 [SMILES: Cc1c(N2C[C@@H](C)O[C@@H](C)C2)nc2c([C@@H](C)Nc3ccccc3C(=O)O)cc(C(F)(F)F)cn2c1=O] | 907 |
| 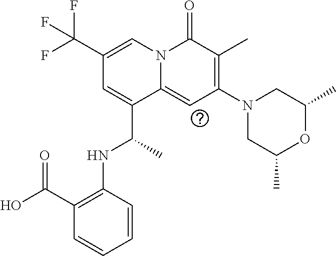 [SMILES: Cc1c(N2C[C@@H](C)O[C@@H](C)C2)cc2c([C@H](C)Nc3ccccc3C(=O)O)cc(C(F)(F)F)cn2c1=O] | 908 |
| 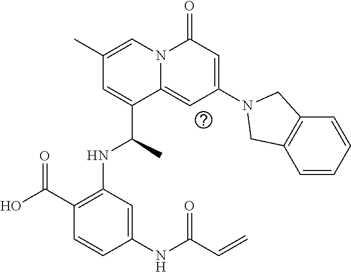 [SMILES: C=CC(=O)Nc1ccc(C(=O)O)c(N[C@H](C)c2cc(C)cn3c(=O)cc(N4Cc5ccccc5C4)cc23)c1] | 909 |
| 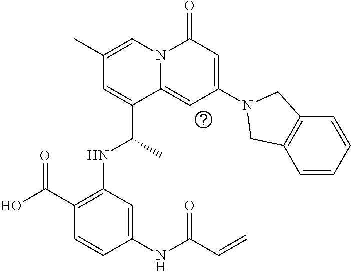 [SMILES: C=CC(=O)Nc1ccc(C(=O)O)c(N[C@@H](C)c2cc(C)cn3c(=O)cc(N4Cc5ccccc5C4)cc23)c1] | 910 |
| 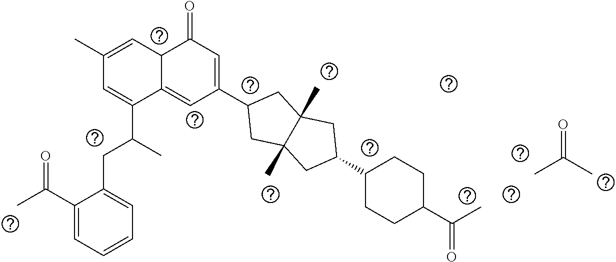 | 911 |
| 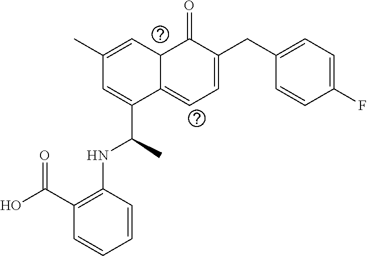 [SMILES: CC1=CC2C(=O)C(Cc3ccc(F)cc3)=CC=C2C([C@@H](C)Nc2ccccc2C(=O)O)=C1] | 912 |
| 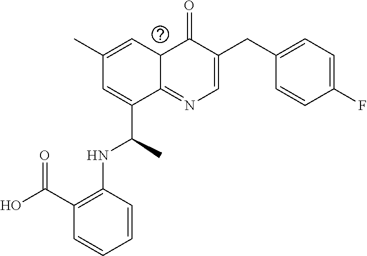 [SMILES: CC1=CC2C(=O)C(Cc3ccc(F)cc3)=CN=C2C([C@@H](C)Nc2ccccc2C(=O)O)=C1] | 913 |
| 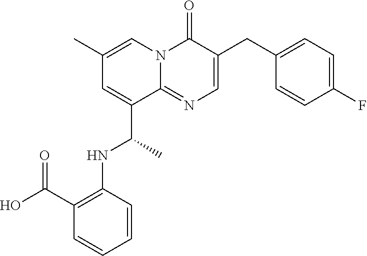 [SMILES: Cc1cc([C@H](C)Nc2ccccc2C(=O)O)c2ncc(Cc3ccc(F)cc3)c(=O)n2c1] | 914 |
| 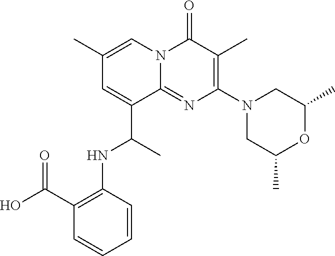 [SMILES: Cc1cc(C(C)Nc2ccccc2C(=O)O)c2nc(N3C[C@@H](C)O[C@@H](C)C3)c(C)c(=O)n2c1] | 915 |
| 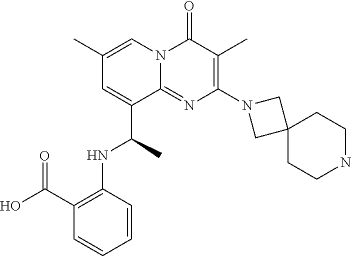 [SMILES: Cc1cc([C@@H](C)Nc2ccccc2C(=O)O)c2nc(N3CC4(CCNCC4)C3)c(C)c(=O)n2c1] | 916 |
| 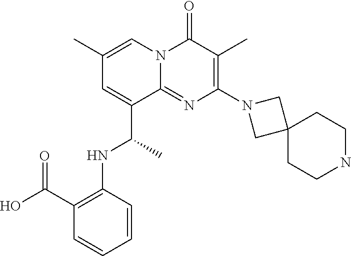 [SMILES: Cc1cc([C@H](C)Nc2ccccc2C(=O)O)c2nc(N3CC4(CCNCC4)C3)c(C)c(=O)n2c1] | 917 |
| 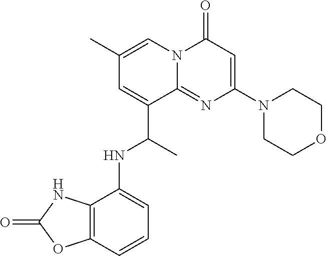 [SMILES: Cc1cc(C(C)Nc2cccc3oc(=O)[nH]c23)c2nc(N3CCOCC3)cc(=O)n2c1] | 918 |
| 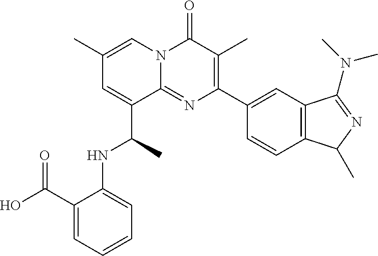 [SMILES: Cc1cc([C@@H](C)Nc2ccccc2C(=O)O)c2nc(-c3ccc4c(c3)C(N(C)C)=NC4C)c(C)c(=O)n2c1] | 919 |
| 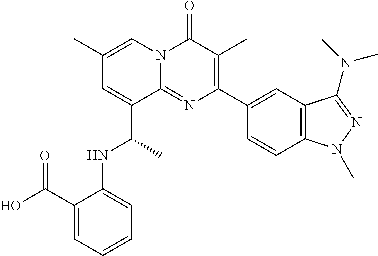 [SMILES: Cc1cc([C@H](C)Nc2ccccc2C(=O)O)c2nc(-c3ccc4c(c3)c(N(C)C)nn4C)c(C)c(=O)n2c1] | 920 |
| 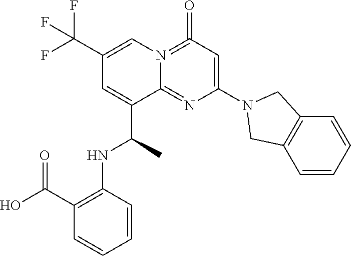 | 921 |
| 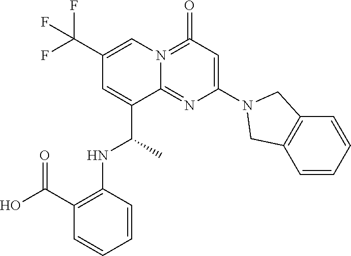 | 922 |
| 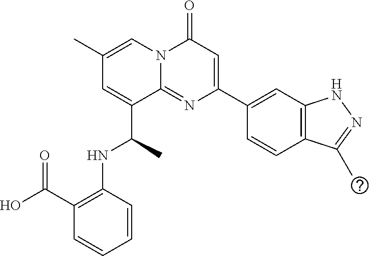 [SMILES: Cc1cc([C@@H](C)Nc2ccccc2C(=O)O)c2nc(-c3ccc4c(C)n[nH]c4c3)cc(=O)n2c1] | 923 |
| 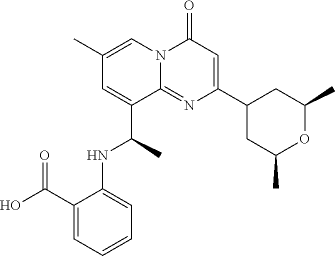 [SMILES: Cc1cc([C@@H](C)Nc2ccccc2C(=O)O)c2nc(C3C[C@@H](C)O[C@@H](C)C3)cc(=O)n2c1] | 924 |
| 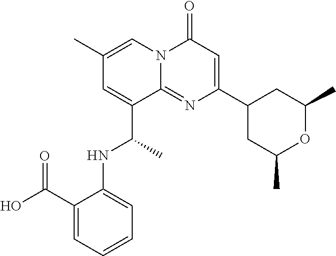 [SMILES: Cc1cc([C@H](C)Nc2ccccc2C(=O)O)c2nc(C3C[C@@H](C)O[C@@H](C)C3)cc(=O)n2c1] | 925 |
| 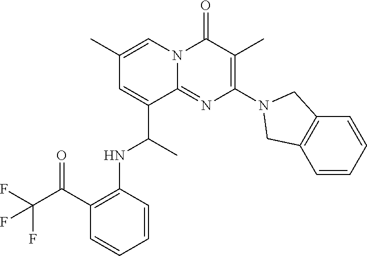 [SMILES: Cc1cc(C(C)Nc2ccccc2C(=O)C(F)(F)F)c2nc(N3Cc4ccccc4C3)c(C)c(=O)n2c1] | 926 |
| 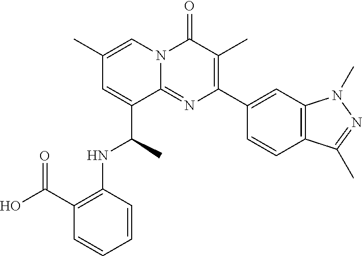 [SMILES: Cc1cc([C@@H](C)Nc2ccccc2C(=O)O)c2nc(-c3ccc4c(C)nn(C)c4c3)c(C)c(=O)n2c1] | 927 |
| 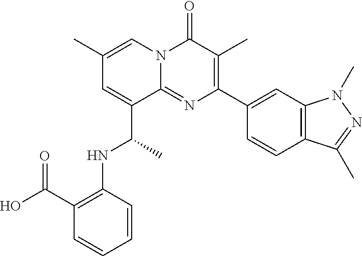 [SMILES: Cc1cc([C@H](C)Nc2ccccc2C(=O)O)c2nc(-c3ccc4c(C)nn(C)c4c3)c(C)c(=O)n2c1] | 928 |
| 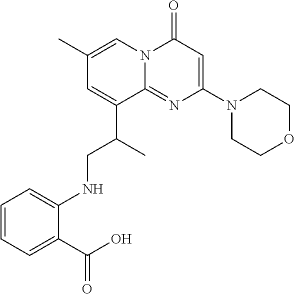 [SMILES: Cc1cc(C(C)CNc2ccccc2C(=O)O)c2nc(N3CCOCC3)cc(=O)n2c1] | 929 |
| 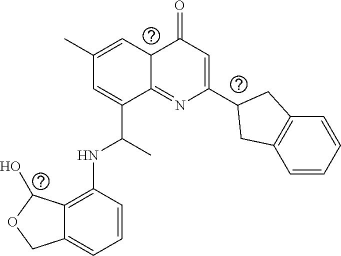 [SMILES: CC1=CC2C(=O)C=C(C3Cc4ccccc4C3)N=C2C(C(C)Nc2cccc3c2C(O)OC3)=C1] | 930 |
| 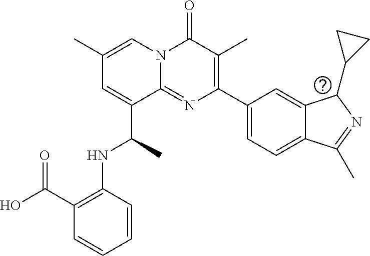 [SMILES: CC1=NC(C2CC2)c2cc(-c3nc4c([C@@H](C)Nc5ccccc5C(=O)O)cc(C)cn4c(=O)c3C)ccc21] | 931 |
| 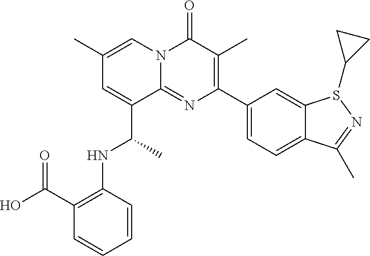 | 932 |
| 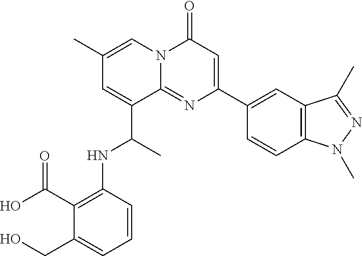 [SMILES: Cc1cc(C(C)Nc2cccc(CO)c2C(=O)O)c2nc(-c3ccc4c(c3)c(C)nn4C)cc(=O)n2c1] | 933 |
| 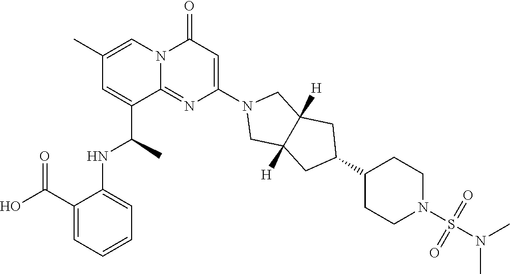 [SMILES: Cc1cc([C@@H](C)Nc2ccccc2C(=O)O)c2nc(N3C[C@H]4C[C@@H](C5CCN(S(=O)(=O)N(C)C)CC5)C[C@H]4C3)cc(=O)n2c1] | 934 |
| 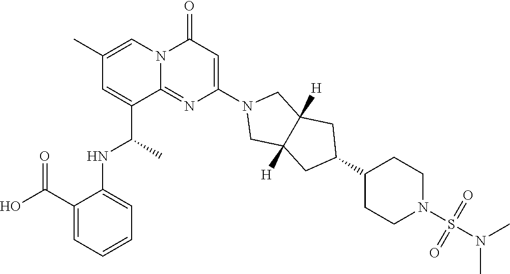 [SMILES: Cc1cc([C@H](C)Nc2ccccc2C(=O)O)c2nc(N3C[C@H]4C[C@@H](C5CCN(S(=O)(=O)N(C)C)CC5)C[C@H]4C3)cc(=O)n2c1] | 935 |
| 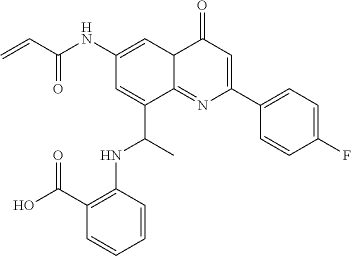 [SMILES: C=CC(=O)NC1=CC2C(=O)C=C(c3ccc(F)cc3)N=C2C(C(C)Nc2ccccc2C(=O)O)=C1] | 936 |
| 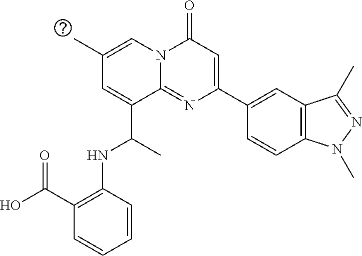 [SMILES: Cc1cc(C(C)Nc2ccccc2C(=O)O)c2nc(-c3ccc4c(c3)c(C)nn4C)cc(=O)n2c1] | 937 |
| 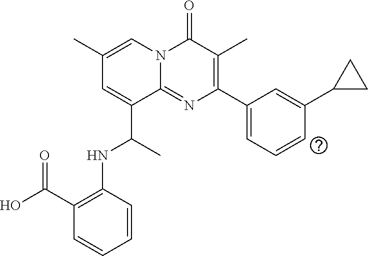 [SMILES: Cc1cc(C(C)Nc2ccccc2C(=O)O)c2nc(-c3cccc(C4CC4)c3)c(C)c(=O)n2c1] | 938 |
| 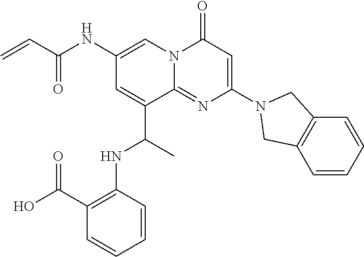 [SMILES: C=CC(=O)Nc1cc(C(C)Nc2ccccc2C(=O)O)c2nc(N3Cc4ccccc4C3)cc(=O)n2c1] | 939 |
| 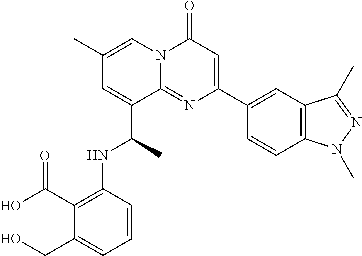 [SMILES: Cc1cc([C@@H](C)Nc2cccc(CO)c2C(=O)O)c2nc(-c3ccc4c(c3)c(C)nn4C)cc(=O)n2c1] | 940 |
| 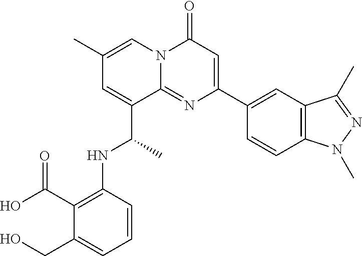 [SMILES: Cc1cc([C@H](C)Nc2cccc(CO)c2C(=O)O)c2nc(-c3ccc4c(c3)c(C)nn4C)cc(=O)n2c1] | 941 |
| 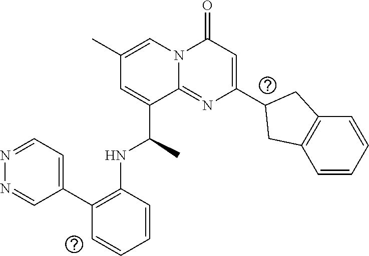 [SMILES: Cc1cc([C@@H](C)Nc2ccccc2-c2ccnnc2)c2nc(C3Cc4ccccc4C3)cc(=O)n2c1] | 942 |
| 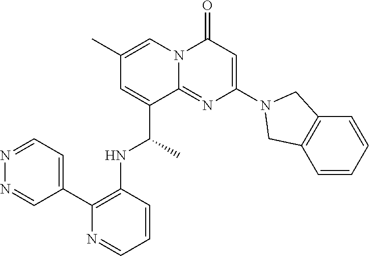 [SMILES: Cc1cc([C@H](C)Nc2cccnc2-c2ccnnc2)c2nc(N3Cc4ccccc4C3)cc(=O)n2c1] | 943 |
| 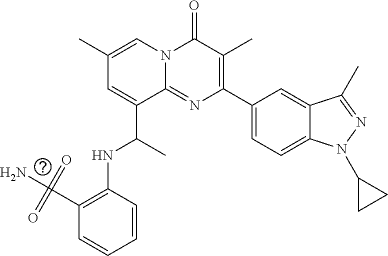 | 944 |
| 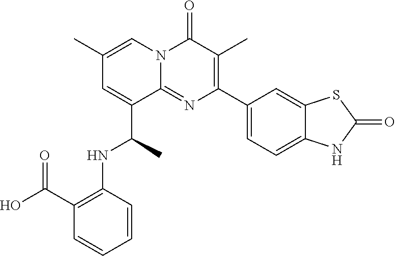 [SMILES: Cc1cc([C@@H](C)Nc2ccccc2C(=O)O)c2nc(-c3ccc4[nH]c(=O)sc4c3)c(C)c(=O)n2c1] | 945 |
| 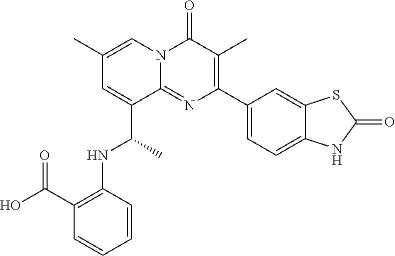 [SMILES: Cc1cc([C@H](C)Nc2ccccc2C(=O)O)c2nc(-c3ccc4[nH]c(=O)sc4c3)c(C)c(=O)n2c1] | 946 |
| 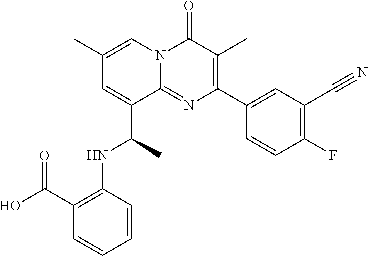 [SMILES: Cc1cc([C@@H](C)Nc2ccccc2C(=O)O)c2nc(-c3ccc(F)c(C#N)c3)c(C)c(=O)n2c1] | 947 |
| 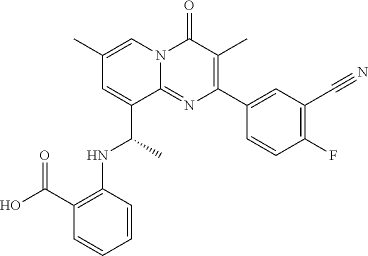 [SMILES: Cc1cc([C@H](C)Nc2ccccc2C(=O)O)c2nc(-c3ccc(F)c(C#N)c3)c(C)c(=O)n2c1] | 948 |
| 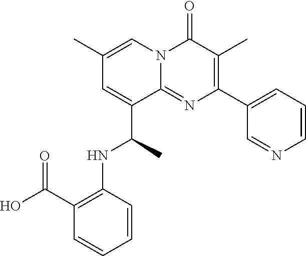 [SMILES: Cc1cc([C@@H](C)Nc2ccccc2C(=O)O)c2nc(-c3cccnc3)c(C)c(=O)n2c1] | 949 |
| 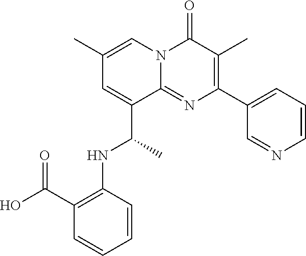 [SMILES: Cc1cc([C@H](C)Nc2ccccc2C(=O)O)c2nc(-c3cccnc3)c(C)c(=O)n2c1] | 950 |
| 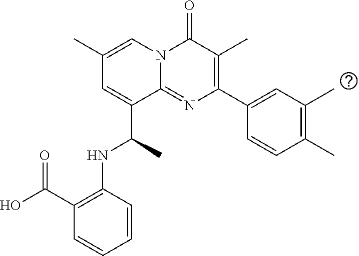 [SMILES: Cc1cc([C@@H](C)Nc2ccccc2C(=O)O)c2nc(-c3ccc(C)c(C)c3)c(C)c(=O)n2c1] | 951 |
| 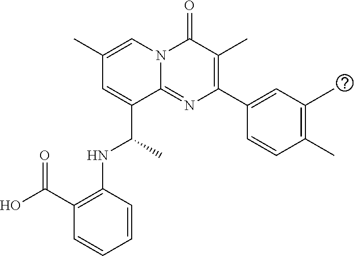 [SMILES: Cc1cc([C@H](C)Nc2ccccc2C(=O)O)c2nc(-c3ccc(C)c(C)c3)c(C)c(=O)n2c1] | 952 |
| 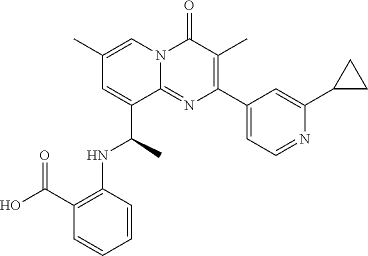 [SMILES: Cc1cc([C@@H](C)Nc2ccccc2C(=O)O)c2nc(-c3ccnc(C4CC4)c3)c(C)c(=O)n2c1] | 953 |
| 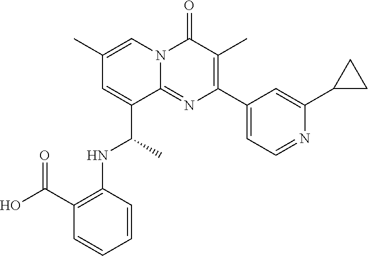 [SMILES: Cc1cc([C@H](C)Nc2ccccc2C(=O)O)c2nc(-c3ccnc(C4CC4)c3)c(C)c(=O)n2c1] | 954 |
| 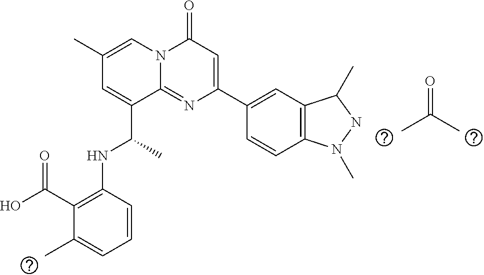 | 955 |
| 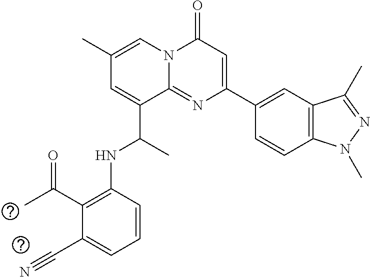 [SMILES: CC(=O)c1c(C#N)cccc1NC(C)c1cc(C)cn2c(=O)cc(-c3ccc4c(c3)c(C)nn4C)nc12] | 956 |
| 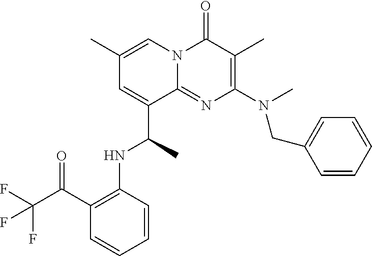 [SMILES: Cc1cc([C@@H](C)Nc2ccccc2C(=O)C(F)(F)F)c2nc(N(C)Cc3ccccc3)c(C)c(=O)n2c1] | 957 |
| 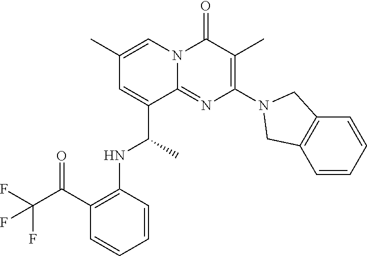 [SMILES: Cc1cc([C@H](C)Nc2ccccc2C(=O)C(F)(F)F)c2nc(N3Cc4ccccc4C3)c(C)c(=O)n2c1] | 958 |
| 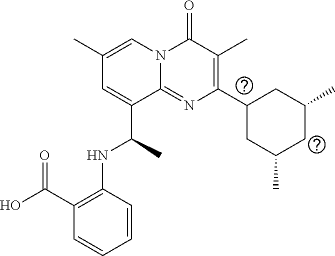 [SMILES: Cc1cc([C@@H](C)Nc2ccccc2C(=O)O)c2nc(C3C[C@@H](C)C[C@@H](C)C3)c(C)c(=O)n2c1] | 959 |
| 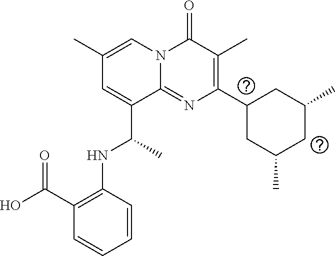 [SMILES: Cc1cc([C@H](C)Nc2ccccc2C(=O)O)c2nc(C3C[C@@H](C)C[C@@H](C)C3)c(C)c(=O)n2c1] | 960 |
| 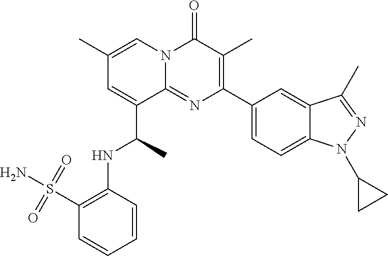 [SMILES: Cc1cc([C@@H](C)Nc2ccccc2S(N)(=O)=O)c2nc(-c3ccc4c(c3)c(C)nn4C3CC3)c(C)c(=O)n2c1] | 961 |
| 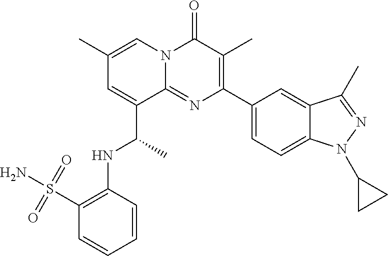 [SMILES: Cc1cc([C@H](C)Nc2ccccc2S(N)(=O)=O)c2nc(-c3ccc4c(c3)c(C)nn4C3CC3)c(C)c(=O)n2c1] | 962 |
| 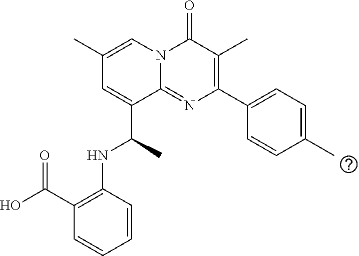 [SMILES: Cc1ccc(-c2nc3c([C@@H](C)Nc4ccccc4C(=O)O)cc(C)cn3c(=O)c2C)cc1] | 963 |
| 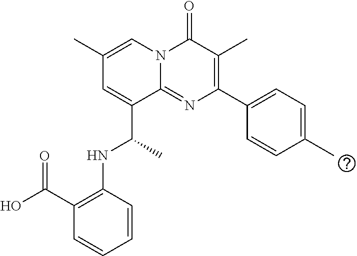 [SMILES: Cc1ccc(-c2nc3c([C@H](C)Nc4ccccc4C(=O)O)cc(C)cn3c(=O)c2C)cc1] | 964 |
| 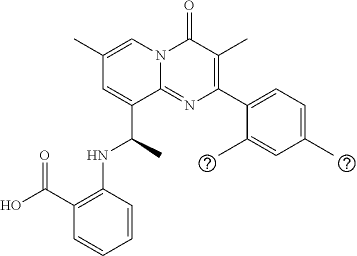 [SMILES: Cc1ccc(-c2nc3c([C@@H](C)Nc4ccccc4C(=O)O)cc(C)cn3c(=O)c2C)c(C)c1] | 965 |
| 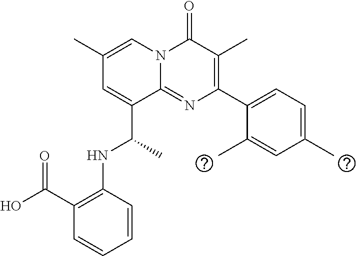 [SMILES: Cc1ccc(-c2nc3c([C@H](C)Nc4ccccc4C(=O)O)cc(C)cn3c(=O)c2C)c(C)c1] | 966 |
| 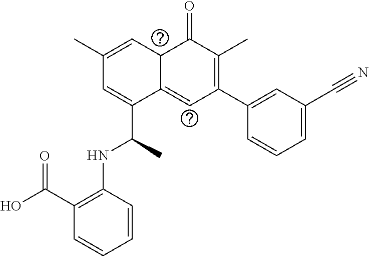 [SMILES: CC1=CC2C(=O)C(C)=C(c3cccc(C#N)c3)C=C2C([C@@H](C)Nc2ccccc2C(=O)O)=C1] | 967 |
| 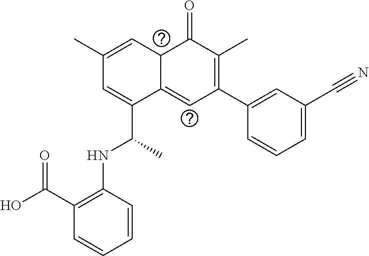 [SMILES: CC1=CC2C(=O)C(C)=C(c3cccc(C#N)c3)C=C2C([C@H](C)Nc2ccccc2C(=O)O)=C1] | 968 |
| 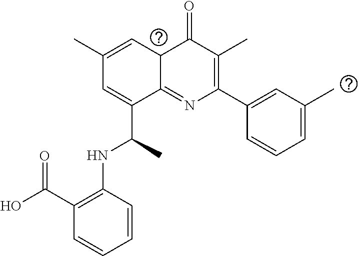 [SMILES: CC1=CC2C(=O)C(C)=C(c3cccc(C)c3)N=C2C([C@@H](C)Nc2ccccc2C(=O)O)=C1] | 969 |
| 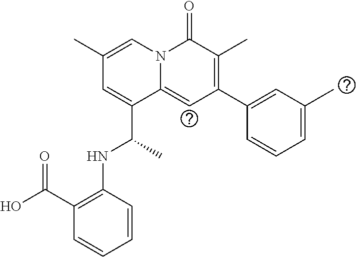 [SMILES: Cc1cccc(-c2cc3c([C@H](C)Nc4ccccc4C(=O)O)cc(C)cn3c(=O)c2C)c1] | 970 |
| 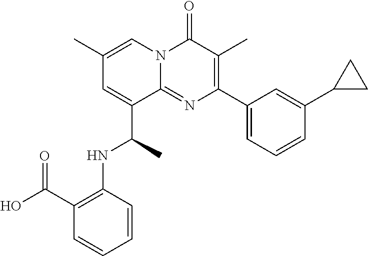 [SMILES: Cc1cc([C@@H](C)Nc2ccccc2C(=O)O)c2nc(-c3cccc(C4CC4)c3)c(C)c(=O)n2c1] | 971 |
| 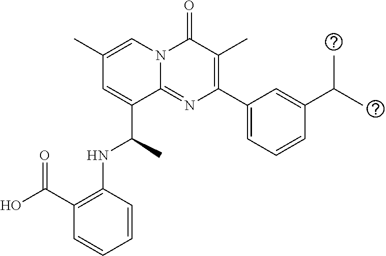 [SMILES: Cc1cc([C@@H](C)Nc2ccccc2C(=O)O)c2nc(-c3cccc(C(C)C)c3)c(C)c(=O)n2c1] | 972 |
| 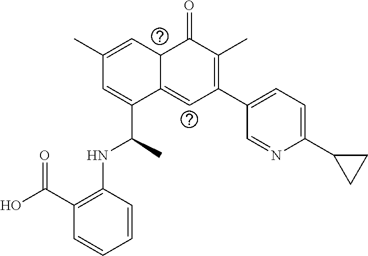 [SMILES: CC1=CC2C(=O)C(C)=C(c3ccc(C4CC4)nc3)C=C2C([C@@H](C)Nc2ccccc2C(=O)O)=C1] | 973 |
| 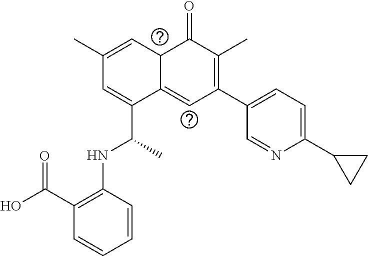 [SMILES: CC1=CC2C(=O)C(C)=C(c3ccc(C4CC4)nc3)C=C2C([C@H](C)Nc2ccccc2C(=O)O)=C1] | 974 |
| 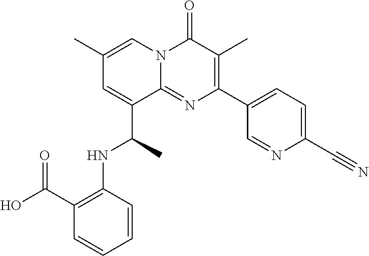 [SMILES: Cc1cc([C@@H](C)Nc2ccccc2C(=O)O)c2nc(-c3ccc(C#N)nc3)c(C)c(=O)n2c1] | 975 |
| 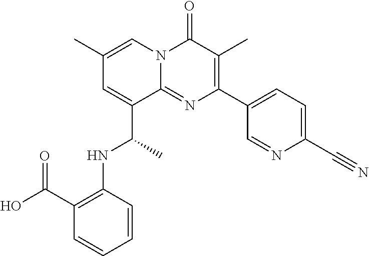 [SMILES: Cc1cc([C@H](C)Nc2ccccc2C(=O)O)c2nc(-c3ccc(C#N)nc3)c(C)c(=O)n2c1] | 976 |
| 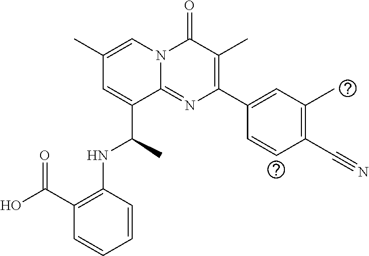 [SMILES: Cc1cc([C@@H](C)Nc2ccccc2C(=O)O)c2nc(-c3ccc(C#N)c(C)c3)c(C)c(=O)n2c1] | 977 |
| 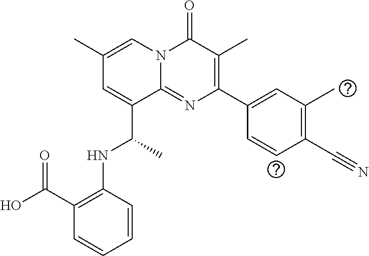 [SMILES: Cc1cc([C@H](C)Nc2ccccc2C(=O)O)c2nc(-c3ccc(C#N)c(C)c3)c(C)c(=O)n2c1] | 978 |
| 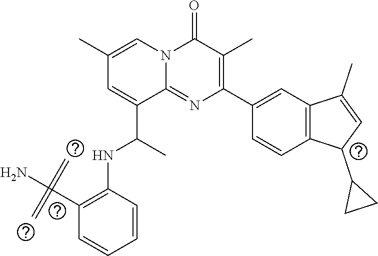 | 979 |
| 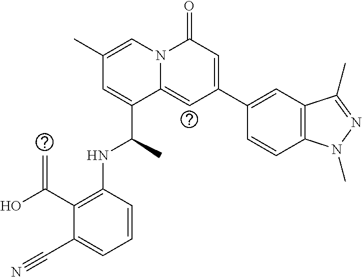 [SMILES: C=C(O)c1c(C#N)cccc1N[C@H](C)c1cc(C)cn2c(=O)cc(-c3ccc4c(c3)c(C)nn4C)cc12] | 980 |
| 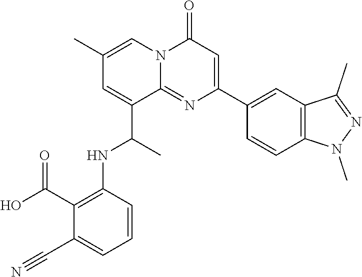 [SMILES: Cc1cc(C(C)Nc2cccc(C#N)c2C(=O)O)c2nc(-c3ccc4c(c3)c(C)nn4C)cc(=O)n2c1] | 981 |
| 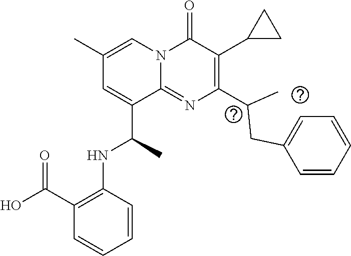 [SMILES: Cc1cc([C@@H](C)Nc2ccccc2C(=O)O)c2nc(C(C)Cc3ccccc3)c(C3CC3)c(=O)n2c1] | 982 |
| 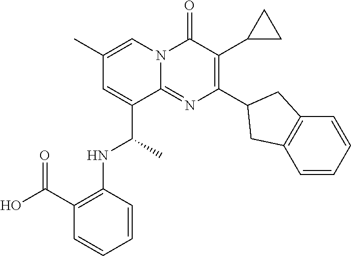 [SMILES: Cc1cc([C@H](C)Nc2ccccc2C(=O)O)c2nc(C3Cc4ccccc4C3)c(C3CC3)c(=O)n2c1] | 983 |
| 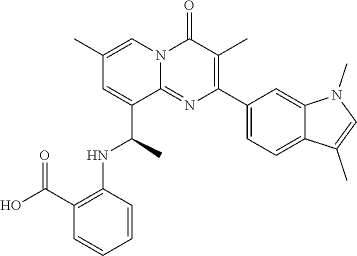 [SMILES: Cc1cc([C@@H](C)Nc2ccccc2C(=O)O)c2nc(-c3ccc4c(C)cn(C)c4c3)c(C)c(=O)n2c1] | 984 |
| 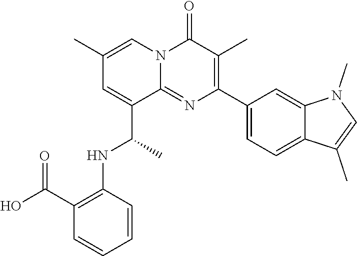 [SMILES: Cc1cc([C@H](C)Nc2ccccc2C(=O)O)c2nc(-c3ccc4c(C)cn(C)c4c3)c(C)c(=O)n2c1] | 985 |
| 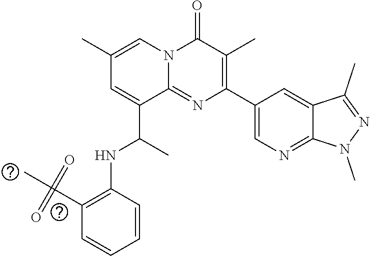 | 986 |
| 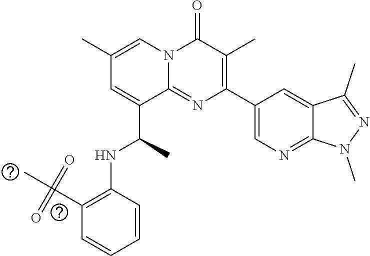 | 987 |
| 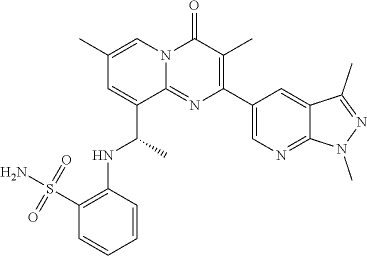 [SMILES: Cc1cc([C@H](C)Nc2ccccc2S(N)(=O)=O)c2nc(-c3cnc4c(c3)c(C)nn4C)c(C)c(=O)n2c1] | 988 |
| 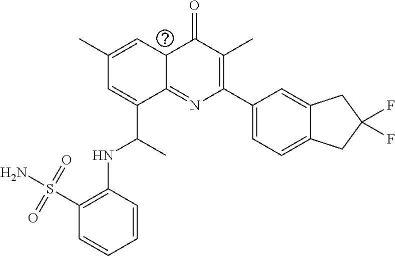 [SMILES: CC1=CC2C(=O)C(C)=C(c3ccc4c(c3)CC(F)(F)C4)N=C2C(C(C)Nc2ccccc2S(N)(=O)=O)=C1] | 989 |
| 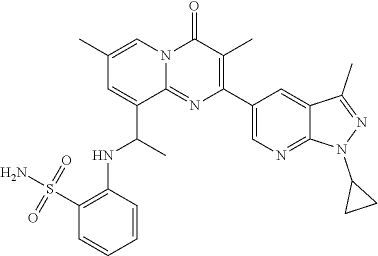 [SMILES: Cc1cc(C(C)Nc2ccccc2S(N)(=O)=O)c2nc(-c3cnc4c(c3)c(C)nn4C3CC3)c(C)c(=O)n2c1] | 990 |
| 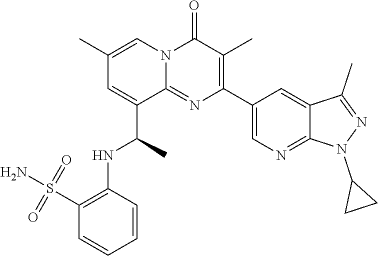 [SMILES: Cc1cc([C@@H](C)Nc2ccccc2S(N)(=O)=O)c2nc(-c3cnc4c(c3)c(C)nn4C3CC3)c(C)c(=O)n2c1] | 991 |
| 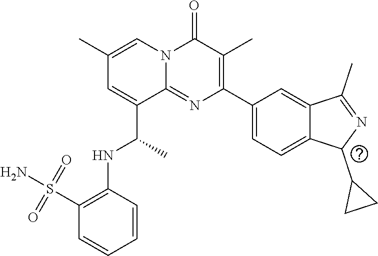 [SMILES: CC1=NC(C2CC2)c2ccc(-c3nc4c([C@H](C)Nc5ccccc5S(N)(=O)=O)cc(C)cn4c(=O)c3C)cc21] | 992 |
| 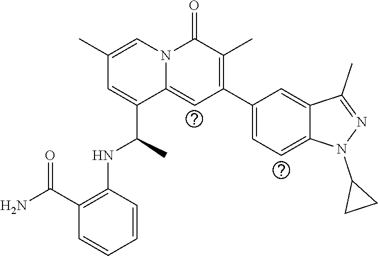 [SMILES: Cc1cc([C@@H](C)Nc2ccccc2C(N)=O)c2cc(-c3ccc4c(c3)c(C)nn4C3CC3)c(C)c(=O)n2c1] | 993 |
| 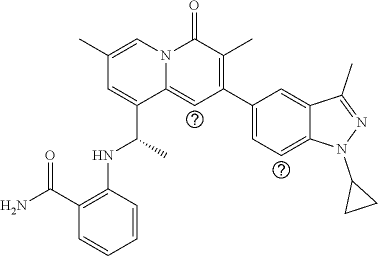 [SMILES: Cc1cc([C@H](C)Nc2ccccc2C(N)=O)c2cc(-c3ccc4c(c3)c(C)nn4C3CC3)c(C)c(=O)n2c1] | 994 |
| 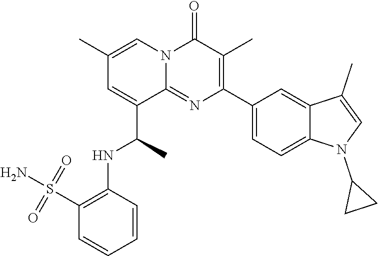 [SMILES: Cc1cc([C@@H](C)Nc2ccccc2S(N)(=O)=O)c2nc(-c3ccc4c(c3)c(C)cn4C3CC3)c(C)c(=O)n2c1] | 995 |
| 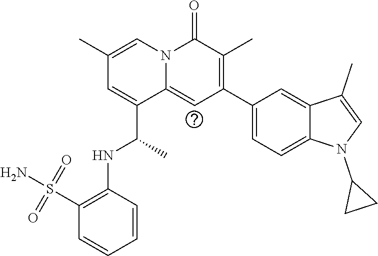 [SMILES: Cc1cc([C@H](C)Nc2ccccc2S(N)(=O)=O)c2cc(-c3ccc4c(c3)c(C)cn4C3CC3)c(C)c(=O)n2c1] | 996 |
| 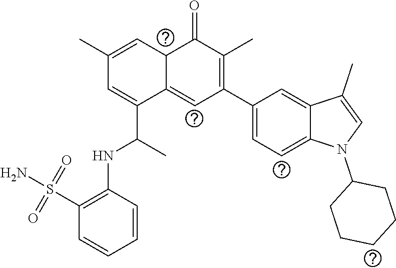 [SMILES: CC1=CC2C(=O)C(C)=C(c3ccc4c(c3)c(C)cn4C3CCCCC3)C=C2C(C(C)Nc2ccccc2S(N)(=O)=O)=C1] | 997 |
| 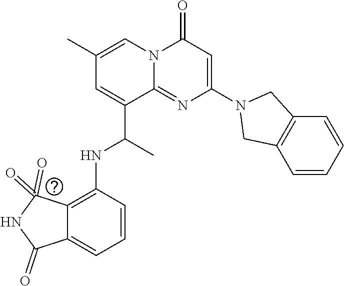 | 998 |
| 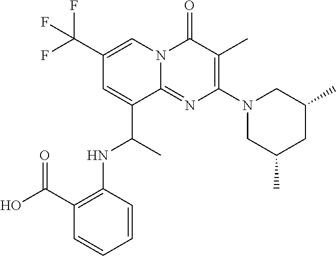 [SMILES: Cc1c(N2C[C@H](C)C[C@H](C)C2)nc2c(C(C)Nc3ccccc3C(=O)O)cc(C(F)(F)F)cn2c1=O] | 999 |
| 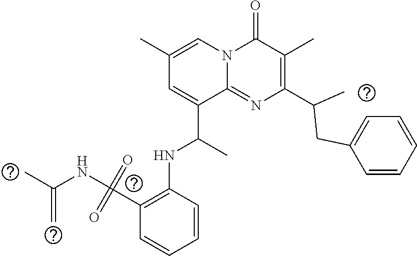 | 1000 |
| 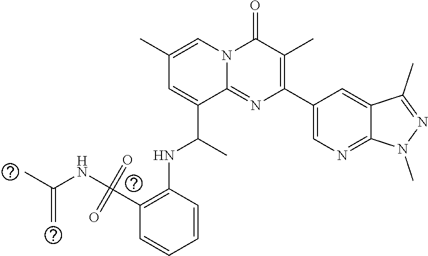 | 1001 |
| 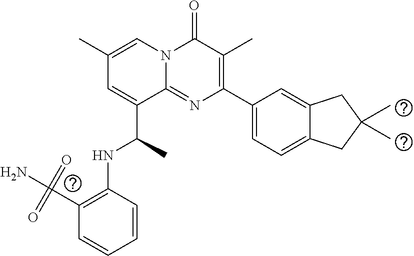 | 1002 |
| 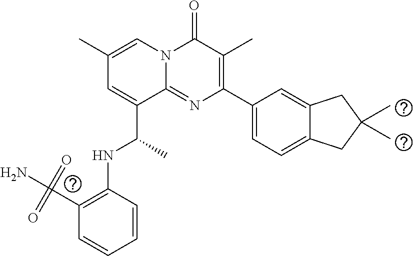 | 1003 |
| 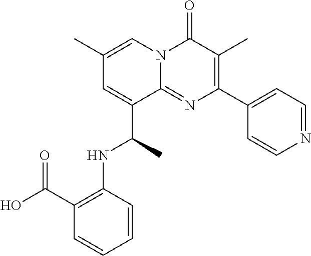 [SMILES: Cc1cc([C@@H](C)Nc2ccccc2C(=O)O)c2nc(-c3ccncc3)c(C)c(=O)n2c1] | 1004 |
| 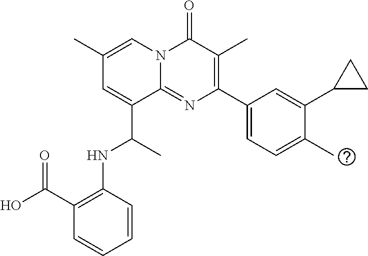 [SMILES: Cc1cc(C(C)Nc2ccccc2C(=O)O)c2nc(-c3ccc(C)c(C4CC4)c3)c(C)c(=O)n2c1] | 1005 |
| 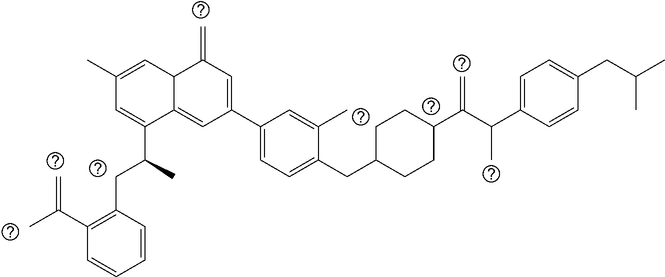 [SMILES: C=C(C)c1ccccc1C[C@H](C)C1=CC(C)=CC2C(=C)C=C(c3ccc(CC4CCC(C(=C)C(C)c5ccc(CC(C)C)cc5)CC4)c(C)c3)C=C12] | 1006 |
| 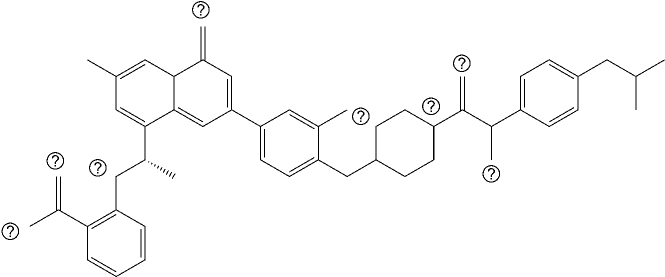 [SMILES: C=C(C)c1ccccc1C[C@@H](C)C1=CC(C)=CC2C(=C)C=C(c3ccc(CC4CCC(C(=C)C(C)c5ccc(CC(C)C)cc5)CC4)c(C)c3)C=C12] | 1007 |
| 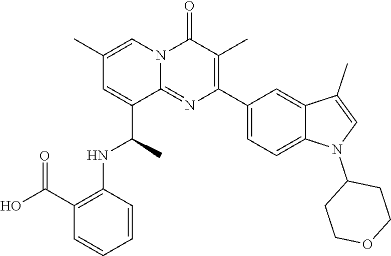 [SMILES: Cc1cc([C@@H](C)Nc2ccccc2C(=O)O)c2nc(-c3ccc4c(c3)c(C)cn4C3CCOCC3)c(C)c(=O)n2c1] | 1008 |
| 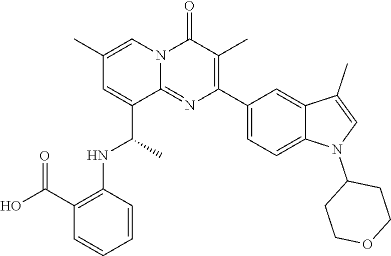 [SMILES: Cc1cc([C@H](C)Nc2ccccc2C(=O)O)c2nc(-c3ccc4c(c3)c(C)cn4C3CCOCC3)c(C)c(=O)n2c1] | 1009 |
| 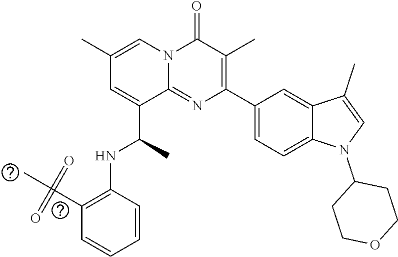 | 1010 |
| 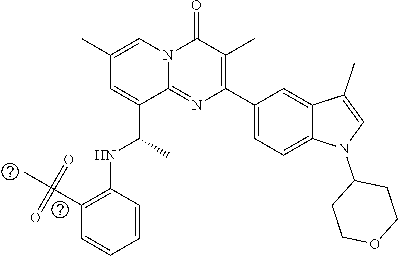 | 1011 |
| 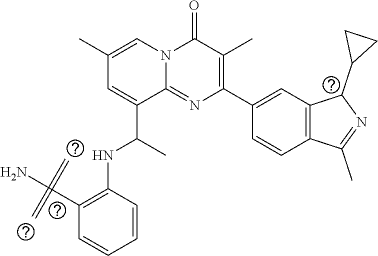 | 1012 |
| 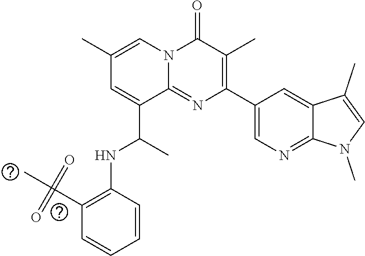 | 1013 |
| 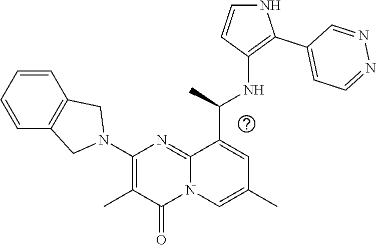 [SMILES: Cc1cc([C@@H](C)Nc2cc[nH]c2-c2ccnnc2)c2nc(N3Cc4ccccc4C3)c(C)c(=O)n2c1] | 1014 |
| 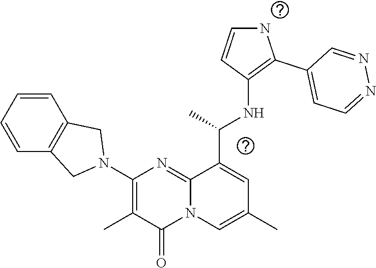 [SMILES: Cc1cc([C@H](C)Nc2cc[nH]c2-c2ccnnc2)c2nc(N3Cc4ccccc4C3)c(C)c(=O)n2c1] | 1015 |
| 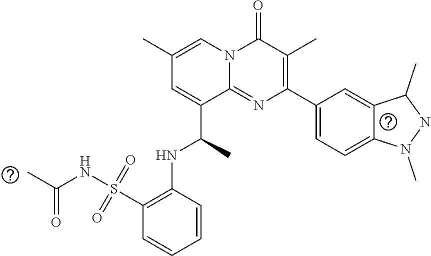 [SMILES: CC(=O)NS(=O)(=O)c1ccccc1N[C@H](C)c1cc(C)cn2c(=O)c(C)c(-c3ccc4c(c3)C(C)NN4C)nc12] | 1016 |
| 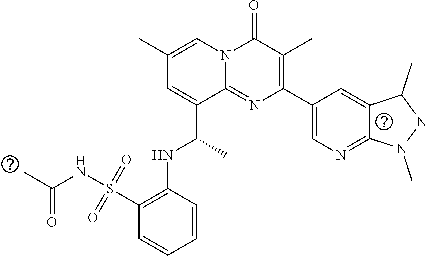 [SMILES: CC(=O)NS(=O)(=O)c1ccccc1N[C@@H](C)c1cc(C)cn2c(=O)c(C)c(-c3cnc4c(c3)C(C)NN4C)nc12] | 1017 |
| 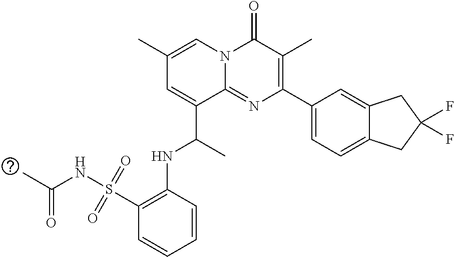 [SMILES: CC(=O)NS(=O)(=O)c1ccccc1NC(C)c1cc(C)cn2c(=O)c(C)c(-c3ccc4c(c3)CC(F)(F)C4)nc12] | 1018 |
| 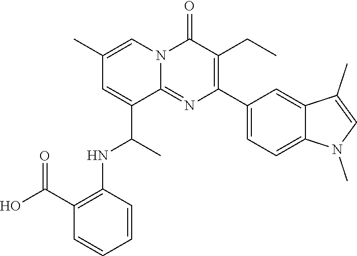 [SMILES: CCc1c(-c2ccc3c(c2)c(C)cn3C)nc2c(C(C)Nc3ccccc3C(=O)O)cc(C)cn2c1=O] | 1019 |
| 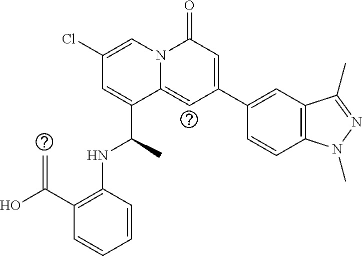 [SMILES: C=C(O)c1ccccc1N[C@H](C)c1cc(Cl)cn2c(=O)cc(-c3ccc4c(c3)c(C)nn4C)cc12] | 1020 |
| 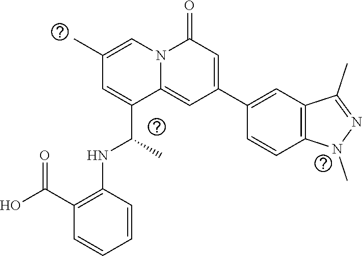 [SMILES: Cc1cc([C@H](C)Nc2ccccc2C(=O)O)c2cc(-c3ccc4c(c3)c(C)nn4C)cc(=O)n2c1] | 1021 |
| 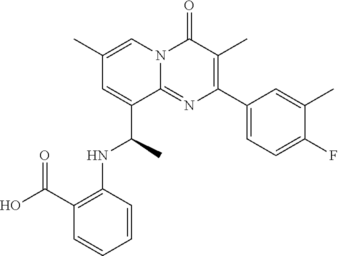 [SMILES: Cc1cc([C@@H](C)Nc2ccccc2C(=O)O)c2nc(-c3ccc(F)c(C)c3)c(C)c(=O)n2c1] | 1022 |
| 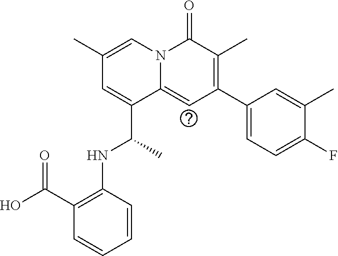 [SMILES: Cc1cc([C@H](C)Nc2ccccc2C(=O)O)c2cc(-c3ccc(F)c(C)c3)c(C)c(=O)n2c1] | 1023 |
| 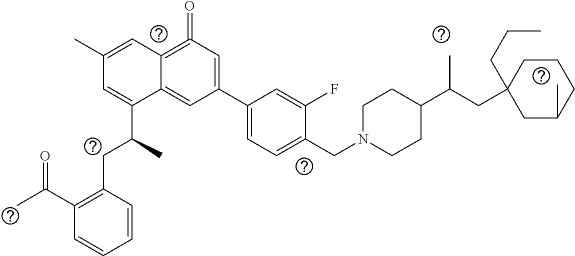 | 1024 |
| 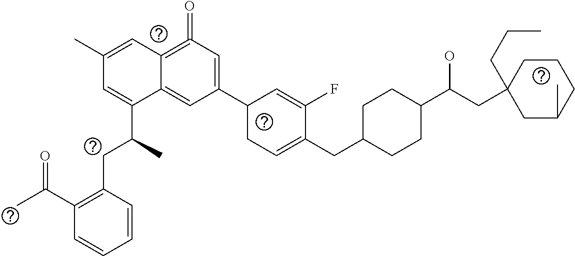 | 1025 |
| 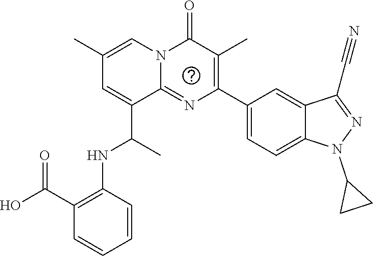 [SMILES: Cc1cc(C(C)Nc2ccccc2C(=O)O)c2nc(-c3ccc4c(c3)c(C#N)nn4C3CC3)c(C)c(=O)n2c1] | 1026 |
| 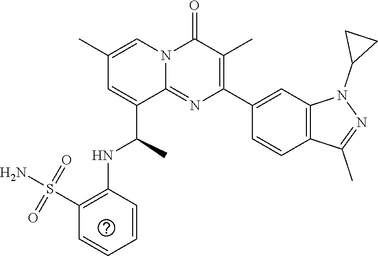 [SMILES: Cc1cc([C@@H](C)Nc2ccccc2S(N)(=O)=O)c2nc(-c3ccc4c(C)nn(C5CC5)c4c3)c(C)c(=O)n2c1] | 1027 |
| 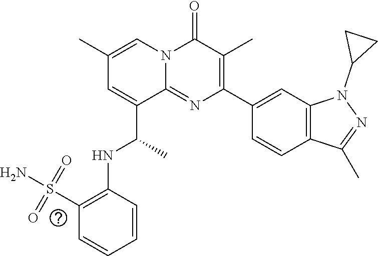 [SMILES: Cc1cc([C@H](C)Nc2ccccc2S(N)(=O)=O)c2nc(-c3ccc4c(C)nn(C5CC5)c4c3)c(C)c(=O)n2c1] | 1028 |
| 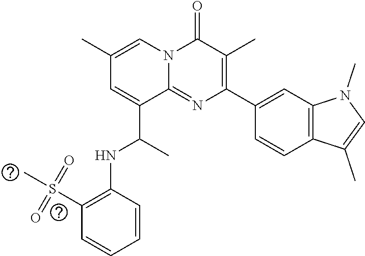 [SMILES: Cc1cc(C(C)Nc2ccccc2S(C)(=O)=O)c2nc(-c3ccc4c(C)cn(C)c4c3)c(C)c(=O)n2c1] | 1029 |
| 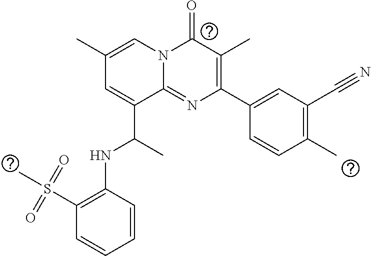 [SMILES: Cc1cc(C(C)Nc2ccccc2S(C)(=O)=O)c2nc(-c3ccc(C)c(C#N)c3)c(C)c(=O)n2c1] | 1030 |
| 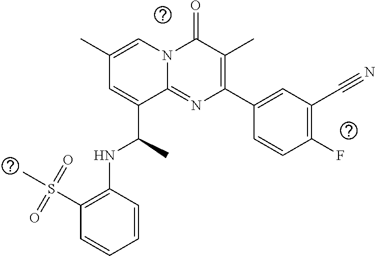 [SMILES: Cc1cc([C@@H](C)Nc2ccccc2S(C)(=O)=O)c2nc(-c3ccc(F)c(C#N)c3)c(C)c(=O)n2c1] | 1031 |
| 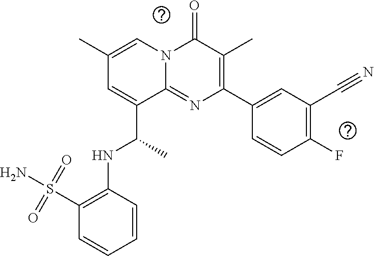 [SMILES: Cc1cc([C@H](C)Nc2ccccc2S(N)(=O)=O)c2nc(-c3ccc(F)c(C#N)c3)c(C)c(=O)n2c1] | 1032 |
| 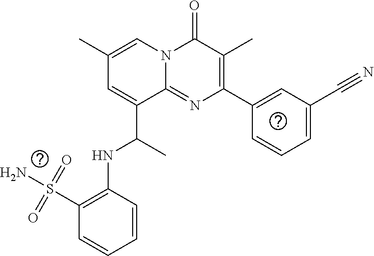 [SMILES: Cc1cc(C(C)Nc2ccccc2S(N)(=O)=O)c2nc(-c3cccc(C#N)c3)c(C)c(=O)n2c1] | 1033 |
| 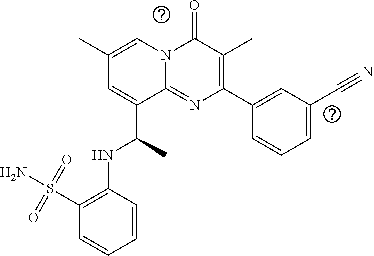 [SMILES: Cc1cc([C@@H](C)Nc2ccccc2S(N)(=O)=O)c2nc(-c3cccc(C#N)c3)c(C)c(=O)n2c1] | 1034 |
| 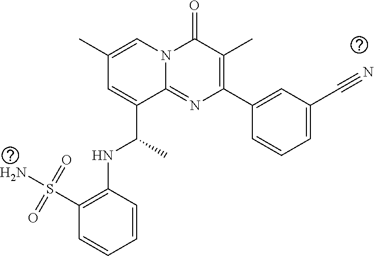 [SMILES: Cc1cc([C@H](C)Nc2ccccc2S(N)(=O)=O)c2nc(-c3cccc(C#N)c3)c(C)c(=O)n2c1] | 1035 |
| 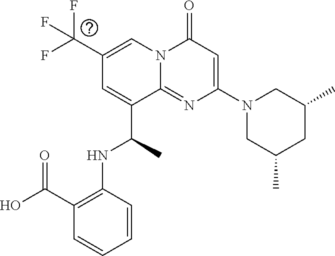 [SMILES: C[C@@H]1C[C@H](C)CN(c2cc(=O)n3cc(C(F)(F)F)cc([C@@H](C)Nc4ccccc4C(=O)O)c3n2)C1] | 1036 |
| 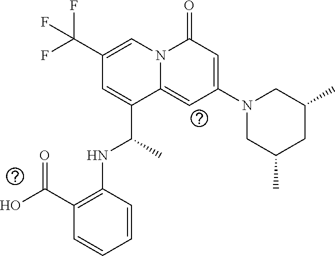 [SMILES: C[C@@H]1C[C@H](C)CN(c2cc(=O)n3cc(C(F)(F)F)cc([C@H](C)Nc4ccccc4C(=O)O)c3c2)C1] | 1037 |
| 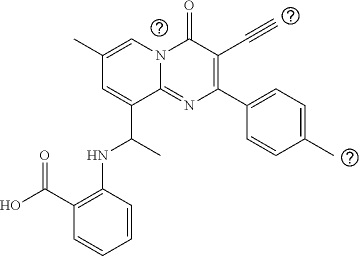 [SMILES: C#Cc1c(-c2ccc(C)cc2)nc2c(C(C)Nc3ccccc3C(=O)O)cc(C)cn2c1=O] | 1038 |
| 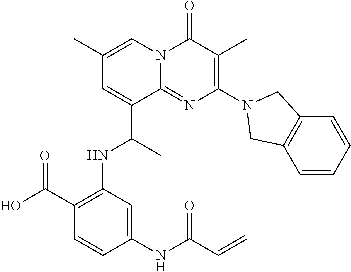 [SMILES: C=CC(=O)Nc1ccc(C(=O)O)c(NC(C)c2cc(C)cn3c(=O)c(C)c(N4Cc5ccccc5C4)nc23)c1] | 1039 |
| 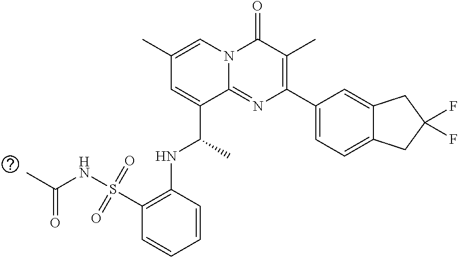 [SMILES: CC(=O)NS(=O)(=O)c1ccccc1N[C@@H](C)c1cc(C)cn2c(=O)c(C)c(-c3ccc4c(c3)CC(F)(F)C4)nc12] | 1040 |
| 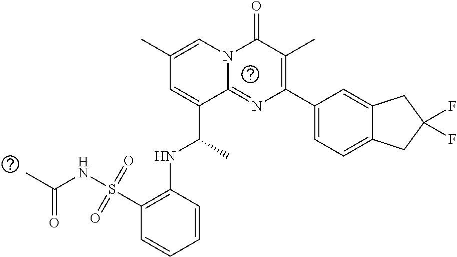 [SMILES: CC(=O)NS(=O)(=O)c1ccccc1N[C@@H](C)c1cc(C)cn2c(=O)c(C)c(-c3ccc4c(c3)CC(F)(F)C4)nc12] | 1041 |
| 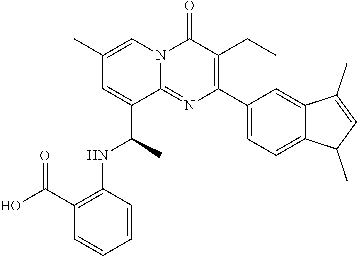 [SMILES: CCc1c(-c2ccc3c(c2)C(C)=CC3C)nc2c([C@@H](C)Nc3ccccc3C(=O)O)cc(C)cn2c1=O] | 1042 |
| 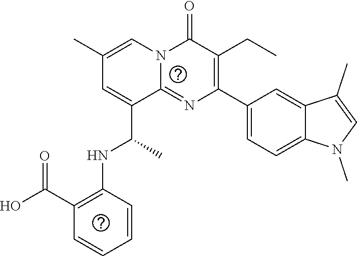 [SMILES: CCc1c(-c2ccc3c(c2)c(C)cn3C)nc2c([C@H](C)Nc3ccccc3C(=O)O)cc(C)cn2c1=O] | 1043 |
| 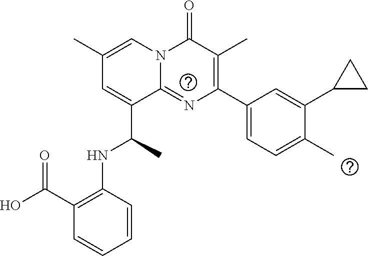 [SMILES: Cc1cc([C@@H](C)Nc2ccccc2C(=O)O)c2nc(-c3ccc(C)c(C4CC4)c3)c(C)c(=O)n2c1] | 1044 |
| 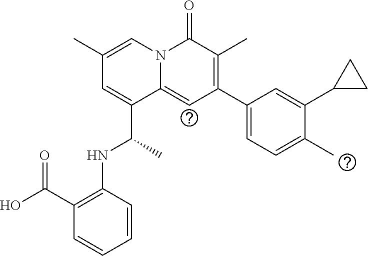 [SMILES: Cc1cc([C@H](C)Nc2ccccc2C(=O)O)c2cc(-c3ccc(C)c(C4CC4)c3)c(C)c(=O)n2c1] | 1045 |
| 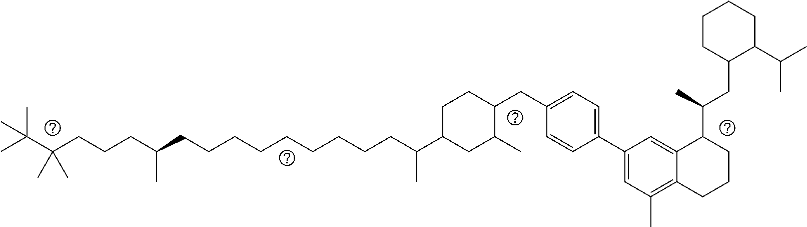 [SMILES: Cc1cc(-c2ccc(CC3CCC(C(C)CCCCCCCCC[C@@H](C)CCCC(C)(C)C(C)(C)C)CC3C)cc2)cc2c1CCCC2[C@@H](C)CC1CCCCC1C(C)C] | 1046 |
| 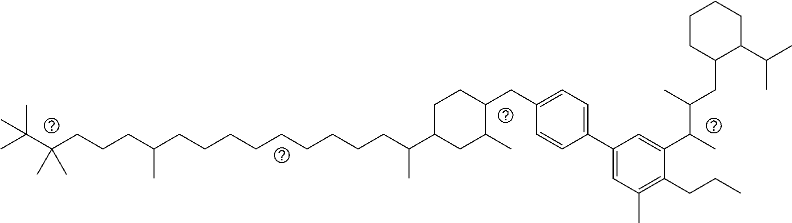 [SMILES: CCCc1c(C)cc(-c2ccc(CC3CCC(C(C)CCCCCCCCCC(C)CCCC(C)(C)C(C)(C)C)CC3C)cc2)cc1C(C)C(C)CC1CCCCC1C(C)C] | 1047 |
| 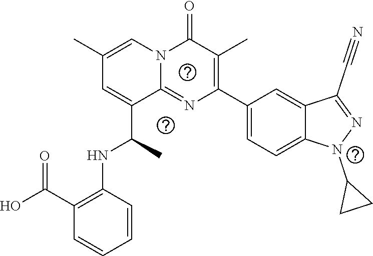 [SMILES: Cc1cc([C@@H](C)Nc2ccccc2C(=O)O)c2nc(-c3ccc4c(c3)c(C#N)nn4C3CC3)c(C)c(=O)n2c1] | 1048 |
| 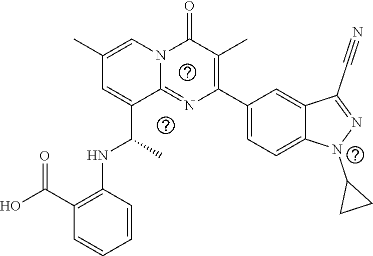 [SMILES: Cc1cc([C@H](C)Nc2ccccc2C(=O)O)c2nc(-c3ccc4c(c3)c(C#N)nn4C3CC3)c(C)c(=O)n2c1] | 1049 |
| 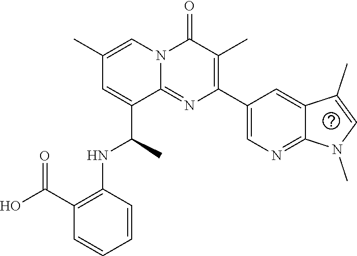 [SMILES: Cc1cc([C@@H](C)Nc2ccccc2C(=O)O)c2nc(-c3cnc4c(c3)c(C)cn4C)c(C)c(=O)n2c1] | 1050 |
| 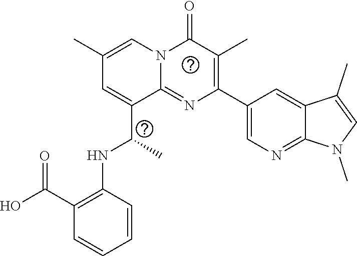 [SMILES: Cc1cc([C@H](C)Nc2ccccc2C(=O)O)c2nc(-c3cnc4c(c3)c(C)cn4C)c(C)c(=O)n2c1] | 1051 |
| 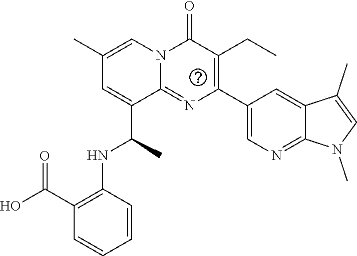 [SMILES: CCc1c(-c2cnc3c(c2)c(C)cn3C)nc2c([C@@H](C)Nc3ccccc3C(=O)O)cc(C)cn2c1=O] | 1052 |
| 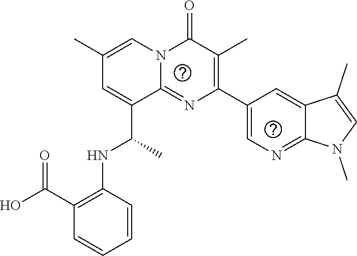 [SMILES: Cc1cc([C@H](C)Nc2ccccc2C(=O)O)c2nc(-c3cnc4c(c3)c(C)cn4C)c(C)c(=O)n2c1] | 1053 |
| 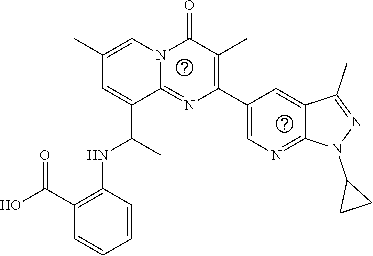 [SMILES: Cc1cc(C(C)Nc2ccccc2C(=O)O)c2nc(-c3cnc4c(c3)c(C)nn4C3CC3)c(C)c(=O)n2c1] | 1054 |
| 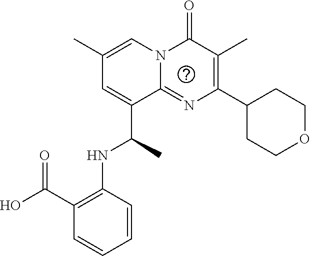 [SMILES: Cc1cc([C@@H](C)Nc2ccccc2C(=O)O)c2nc(C3CCOCC3)c(C)c(=O)n2c1] | 1055 |
| 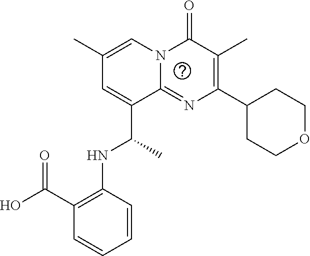 [SMILES: Cc1cc([C@H](C)Nc2ccccc2C(=O)O)c2nc(C3CCOCC3)c(C)c(=O)n2c1] | 1056 |
| 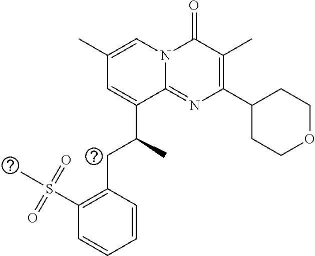 [SMILES: Cc1cc([C@@H](C)Cc2ccccc2S(C)(=O)=O)c2nc(C3CCOCC3)c(C)c(=O)n2c1] | 1057 |
| 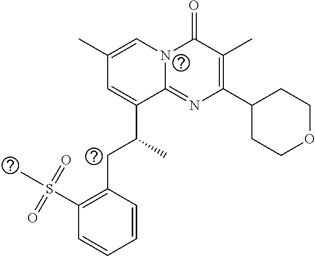 [SMILES: Cc1cc([C@H](C)Cc2ccccc2S(C)(=O)=O)c2nc(C3CCOCC3)c(C)c(=O)n2c1] | 1058 |
| 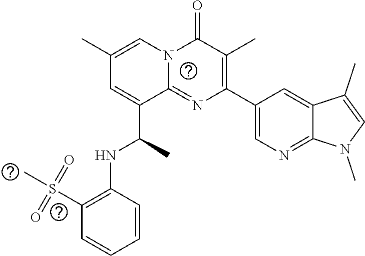 [SMILES: Cc1cc([C@@H](C)Nc2ccccc2S(C)(=O)=O)c2nc(-c3cnc4c(c3)c(C)cn4C)c(C)c(=O)n2c1] | 1059 |
| 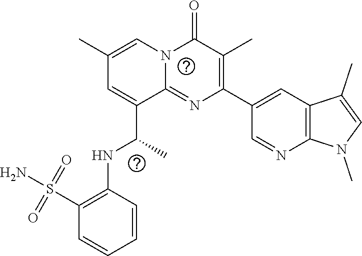 [SMILES: Cc1cc([C@H](C)Nc2ccccc2S(N)(=O)=O)c2nc(-c3cnc4c(c3)c(C)cn4C)c(C)c(=O)n2c1] | 1060 |
| 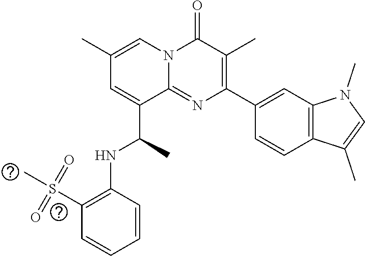 [SMILES: Cc1cc([C@@H](C)Nc2ccccc2S(C)(=O)=O)c2nc(-c3ccc4c(C)cn(C)c4c3)c(C)c(=O)n2c1] | 1061 |
| 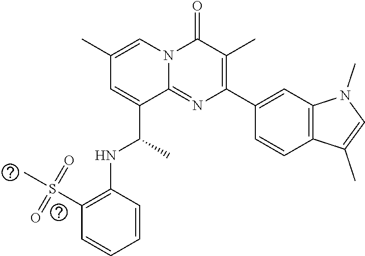 [SMILES: Cc1cc([C@H](C)Nc2ccccc2S(C)(=O)=O)c2nc(-c3ccc4c(C)cn(C)c4c3)c(C)c(=O)n2c1] | 1062 |
| 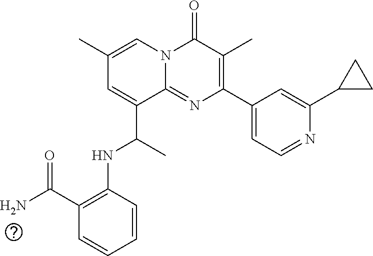 [SMILES: Cc1cc(C(C)Nc2ccccc2C(N)=O)c2nc(-c3ccnc(C4CC4)c3)c(C)c(=O)n2c1] | 1063 |
| 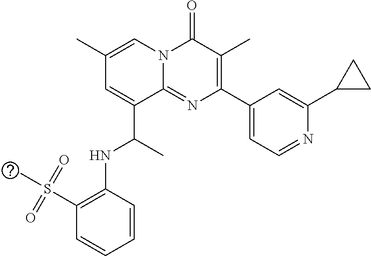 [SMILES: Cc1cc(C(C)Nc2ccccc2S(C)(=O)=O)c2nc(-c3ccnc(C4CC4)c3)c(C)c(=O)n2c1] | 1064 |
| 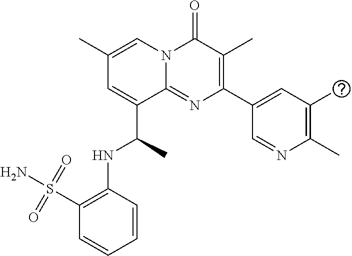 [SMILES: Cc1cc([C@@H](C)Nc2ccccc2S(N)(=O)=O)c2nc(-c3cnc(C)c(C)c3)c(C)c(=O)n2c1] | 1065 |
| 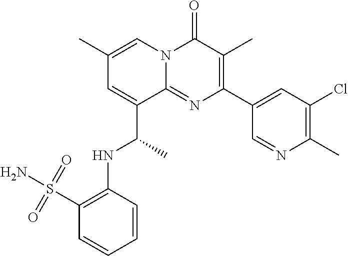 [SMILES: Cc1cc([C@H](C)Nc2ccccc2S(N)(=O)=O)c2nc(-c3cnc(C)c(Cl)c3)c(C)c(=O)n2c1] | 1066 |
| 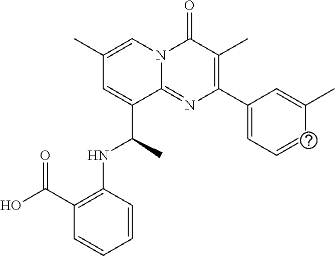 [SMILES: Cc1cccc(-c2nc3c([C@@H](C)Nc4ccccc4C(=O)O)cc(C)cn3c(=O)c2C)c1] | 1067 |
| 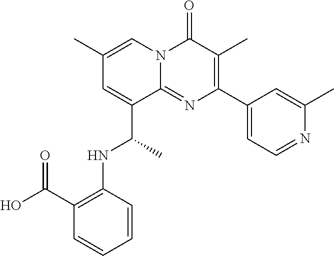 [SMILES: Cc1cc([C@H](C)Nc2ccccc2C(=O)O)c2nc(-c3ccnc(C)c3)c(C)c(=O)n2c1] | 1068 |
| 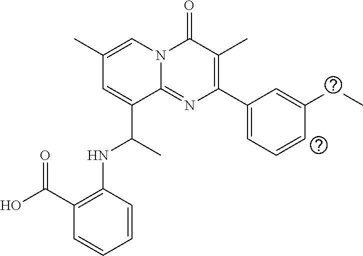 [SMILES: CCc1cccc(-c2nc3c(C(C)Nc4ccccc4C(=O)O)cc(C)cn3c(=O)c2C)c1] | 1069 |
| 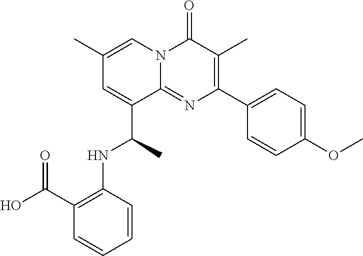 [SMILES: COc1ccc(-c2nc3c([C@@H](C)Nc4ccccc4C(=O)O)cc(C)cn3c(=O)c2C)cc1] | 1070 |
| 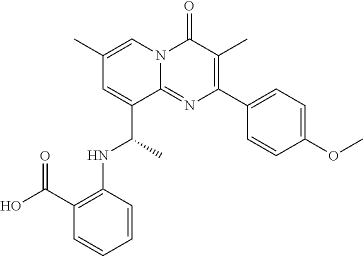 [SMILES: COc1ccc(-c2nc3c([C@H](C)Nc4ccccc4C(=O)O)cc(C)cn3c(=O)c2C)cc1] | 1071 |
| 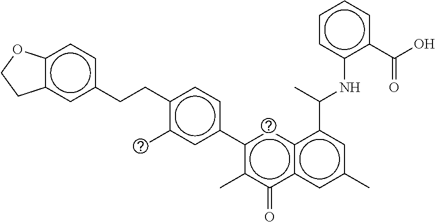 | 1072 |
| 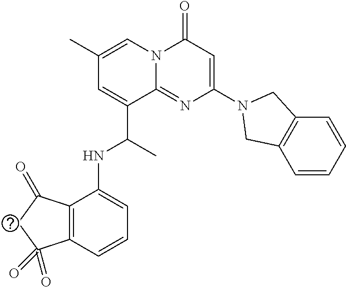 | 1073 |
| 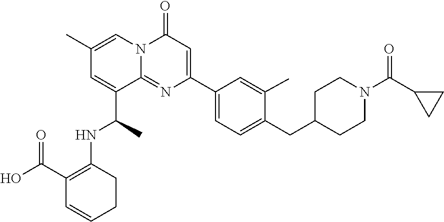 [SMILES: Cc1cc([C@@H](C)NC2=C(C(=O)O)C=CCC2)c2nc(-c3ccc(CC4CCN(C(=O)C5CC5)CC4)c(C)c3)cc(=O)n2c1] | 1074 |
| 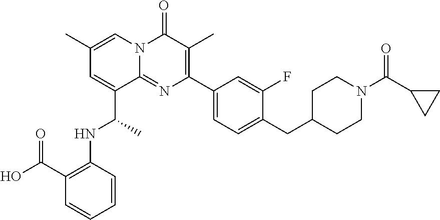 [SMILES: Cc1cc([C@H](C)Nc2ccccc2C(=O)O)c2nc(-c3ccc(CC4CCN(C(=O)C5CC5)CC4)c(F)c3)c(C)c(=O)n2c1] | 1075 |
| 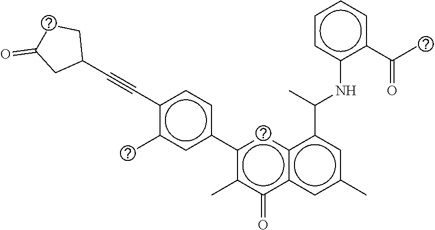 | 1076 |
| 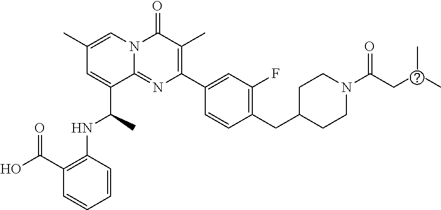 [SMILES: Cc1cc([C@@H](C)Nc2ccccc2C(=O)O)c2nc(-c3ccc(CC4CCN(C(=O)CC(C)C)CC4)c(F)c3)c(C)c(=O)n2c1] | 1077 |
| 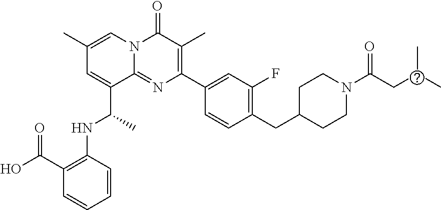 [SMILES: Cc1cc([C@H](C)Nc2ccccc2C(=O)O)c2nc(-c3ccc(CC4CCN(C(=O)CC(C)C)CC4)c(F)c3)c(C)c(=O)n2c1] | 1078 |
| 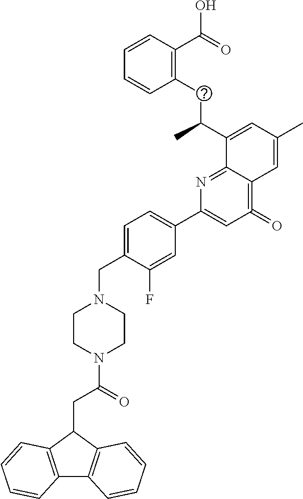 | 1079 |
| 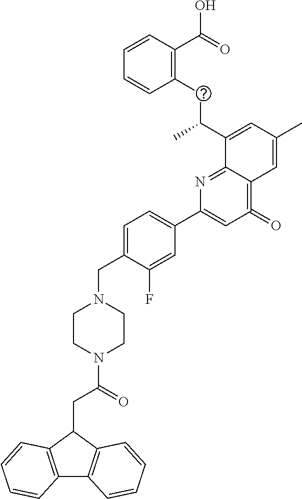 | 1080 |
| 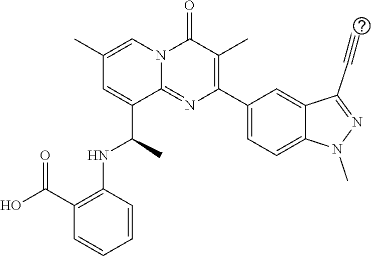 [SMILES: C#Cc1nn(C)c2ccc(-c3nc4c([C@@H](C)Nc5ccccc5C(=O)O)cc(C)cn4c(=O)c3C)cc12] | 1081 |
| 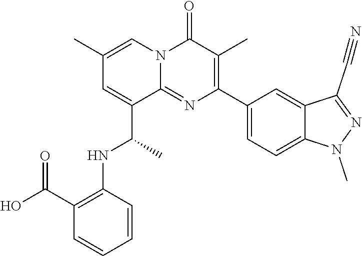 [SMILES: Cc1cc([C@H](C)Nc2ccccc2C(=O)O)c2nc(-c3ccc4c(c3)c(C#N)nn4C)c(C)c(=O)n2c1] | 1082 |
| 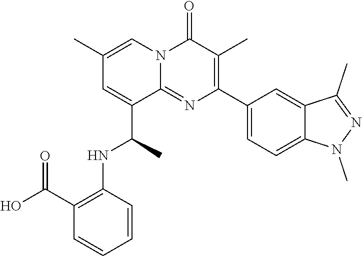 [SMILES: Cc1cc([C@@H](C)Nc2ccccc2C(=O)O)c2nc(-c3ccc4c(c3)c(C)nn4C)c(C)c(=O)n2c1] | 1083 |
| 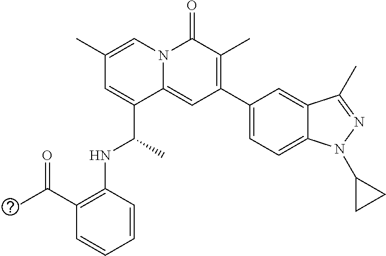 [SMILES: CC(=O)c1ccccc1N[C@@H](C)c1cc(C)cn2c(=O)c(C)c(-c3ccc4c(c3)c(C)nn4C3CC3)cc12] | 1084 |
| 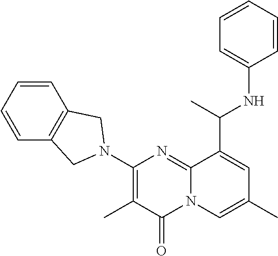 [SMILES: Cc1cc(C(C)Nc2ccccc2)c2nc(N3Cc4ccccc4C3)c(C)c(=O)n2c1] | 1085 |
| 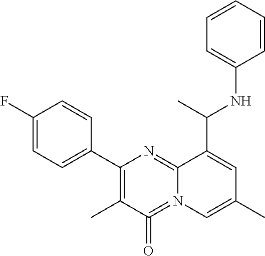 | 1086 |
| 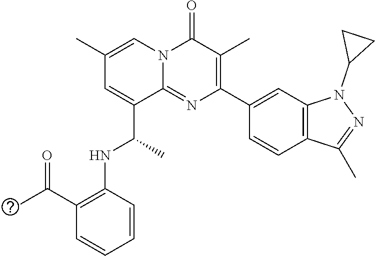 [SMILES: CC(=O)c1ccccc1N[C@@H](C)c1cc(C)cn2c(=O)c(C)c(-c3ccc4c(C)nn(C5CC5)c4c3)nc12] | 1087 |
| 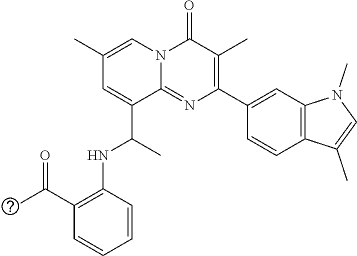 [SMILES: CC(=O)c1ccccc1NC(C)c1cc(C)cn2c(=O)c(C)c(-c3ccc4c(C)cn(C)c4c3)nc12] | 1088 |
| 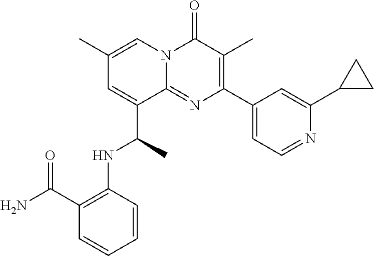 [SMILES: Cc1cc([C@@H](C)Nc2ccccc2C(N)=O)c2nc(-c3ccnc(C4CC4)c3)c(C)c(=O)n2c1] | 1089 |
| 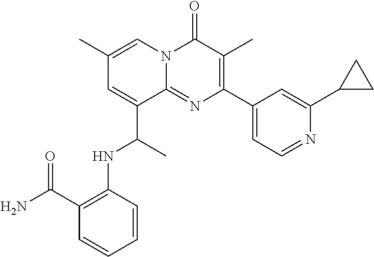 [SMILES: Cc1cc(C(C)Nc2ccccc2C(N)=O)c2nc(-c3ccnc(C4CC4)c3)c(C)c(=O)n2c1] | 1090 |
| 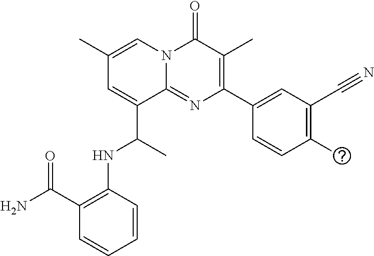 [SMILES: Cc1cc(C(C)Nc2ccccc2C(N)=O)c2nc(-c3ccc(C)c(C#N)c3)c(C)c(=O)n2c1] | 1091 |
| 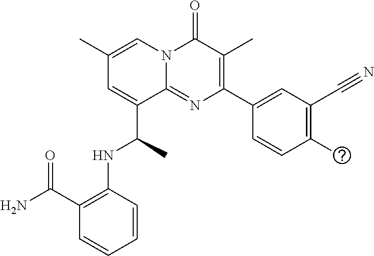 [SMILES: Cc1cc([C@@H](C)Nc2ccccc2C(N)=O)c2nc(-c3ccc(C)c(C#N)c3)c(C)c(=O)n2c1] | 1092 |
| 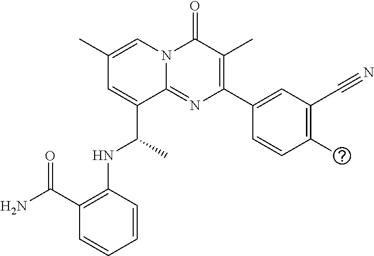 [SMILES: Cc1cc([C@H](C)Nc2ccccc2C(N)=O)c2nc(-c3ccc(C)c(C#N)c3)c(C)c(=O)n2c1] | 1093 |
| 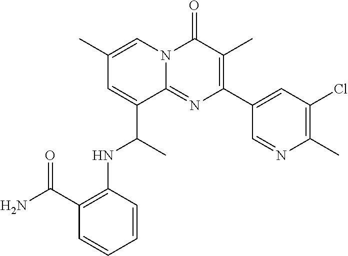 [SMILES: Cc1cc(C(C)Nc2ccccc2C(N)=O)c2nc(-c3cnc(C)c(Cl)c3)c(C)c(=O)n2c1] | 1094 |
| 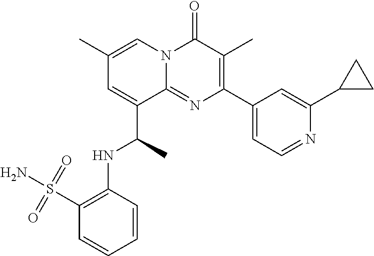 [SMILES: Cc1cc([C@@H](C)Nc2ccccc2S(N)(=O)=O)c2nc(-c3ccnc(C4CC4)c3)c(C)c(=O)n2c1] | 1095 |
| 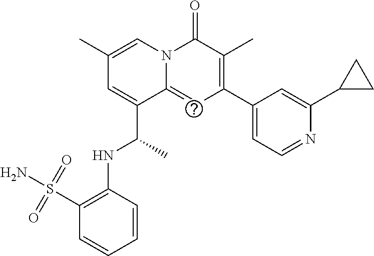 [SMILES: Cc1cc([C@H](C)Nc2ccccc2S(N)(=O)=O)c2cc(-c3ccnc(C4CC4)c3)c(C)c(=O)n2c1] | 1096 |
| 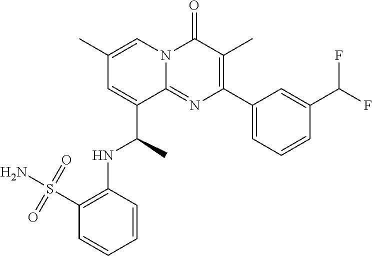 [SMILES: Cc1cc([C@@H](C)Nc2ccccc2S(N)(=O)=O)c2nc(-c3cccc(C(F)F)c3)c(C)c(=O)n2c1] | 1097 |
| 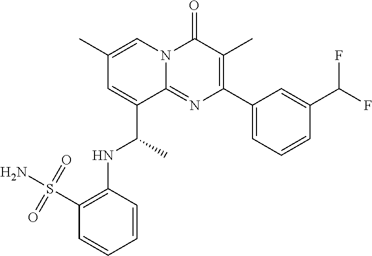 [SMILES: Cc1cc([C@H](C)Nc2ccccc2S(N)(=O)=O)c2nc(-c3cccc(C(F)F)c3)c(C)c(=O)n2c1] | 1098 |
| 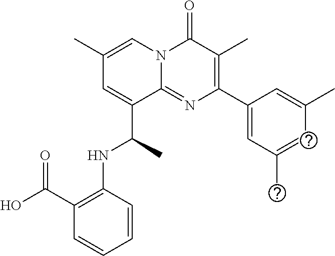 [SMILES: Cc1cc(C)cc(-c2nc3c([C@@H](C)Nc4ccccc4C(=O)O)cc(C)cn3c(=O)c2C)c1] | 1099 |
| 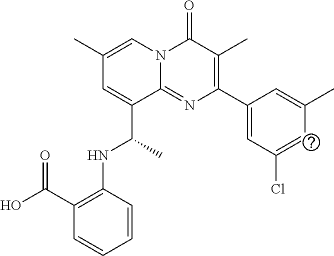 [SMILES: Cc1cc(Cl)cc(-c2nc3c([C@H](C)Nc4ccccc4C(=O)O)cc(C)cn3c(=O)c2C)c1] | 1100 |
| 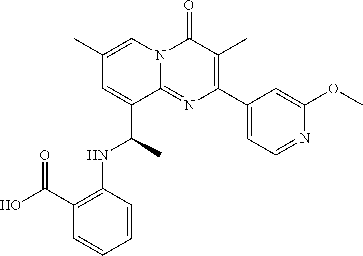 [SMILES: COc1cc(-c2nc3c([C@@H](C)Nc4ccccc4C(=O)O)cc(C)cn3c(=O)c2C)ccn1] | 1101 |
| 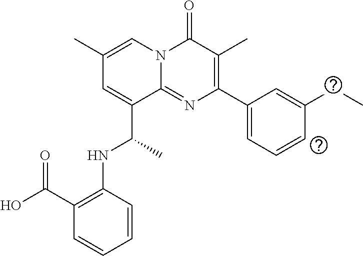 [SMILES: CCc1cccc(-c2nc3c([C@H](C)Nc4ccccc4C(=O)O)cc(C)cn3c(=O)c2C)c1] | 1102 |
| 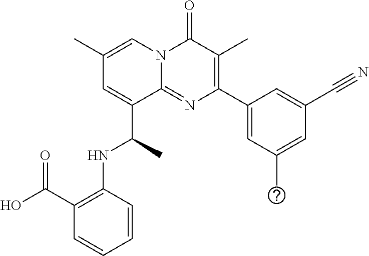 [SMILES: Cc1cc(C#N)cc(-c2nc3c([C@@H](C)Nc4ccccc4C(=O)O)cc(C)cn3c(=O)c2C)c1] | 1103 |
| 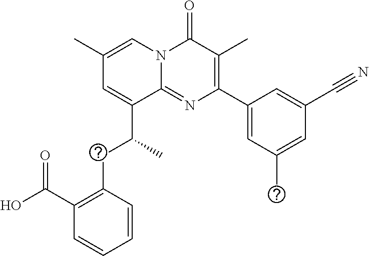 [SMILES: Cc1cc(C#N)cc(-c2nc3c([C@H](C)Cc4ccccc4C(=O)O)cc(C)cn3c(=O)c2C)c1] | 1104 |
| 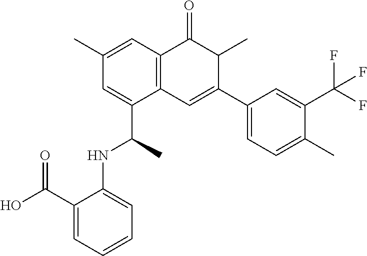 [SMILES: Cc1cc2c(c([C@@H](C)Nc3ccccc3C(=O)O)c1)C=C(c1ccc(C)c(C(F)(F)F)c1)C(C)C2=O] | 1105 |
| 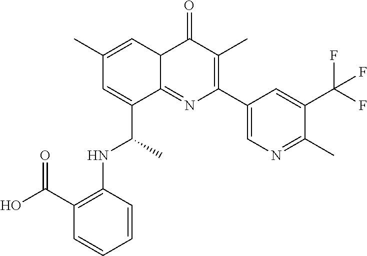 [SMILES: CC1=CC2C(=O)C(C)=C(c3cnc(C)c(C(F)(F)F)c3)N=C2C([C@H](C)Nc2ccccc2C(=O)O)=C1] | 1106 |
| 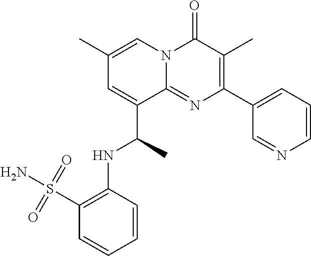 [SMILES: Cc1cc([C@@H](C)Nc2ccccc2S(N)(=O)=O)c2nc(-c3cccnc3)c(C)c(=O)n2c1] | 1107 |
| 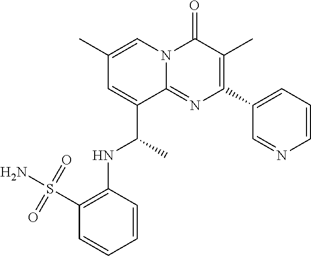 | 1108 |
| 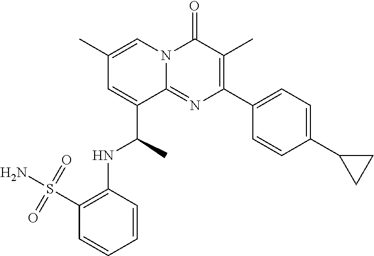 [SMILES: Cc1cc([C@@H](C)Nc2ccccc2S(N)(=O)=O)c2nc(-c3ccc(C4CC4)cc3)c(C)c(=O)n2c1] | 1109 |
| 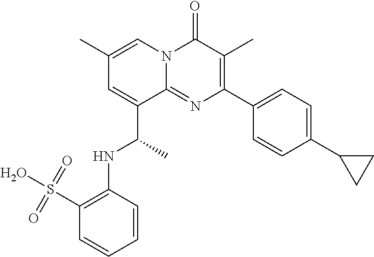 [SMILES: Cc1cc([C@H](C)Nc2ccccc2S(=O)(=O)O)c2nc(-c3ccc(C4CC4)cc3)c(C)c(=O)n2c1] | 1110 |
| 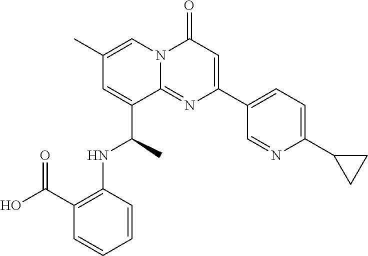 [SMILES: Cc1cc([C@@H](C)Nc2ccccc2C(=O)O)c2nc(-c3ccc(C4CC4)nc3)cc(=O)n2c1] | 1111 |
| 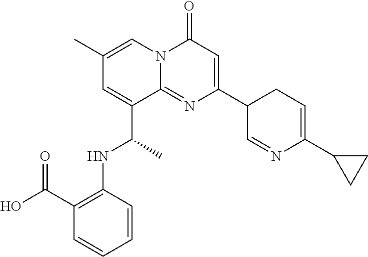 [SMILES: Cc1cc([C@H](C)Nc2ccccc2C(=O)O)c2nc(C3C=NC(C4CC4)=CC3)cc(=O)n2c1] | 1112 |
| 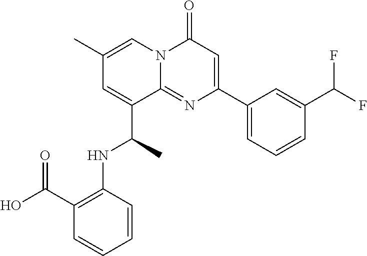 [SMILES: Cc1cc([C@@H](C)Nc2ccccc2C(=O)O)c2nc(-c3cccc(C(F)F)c3)cc(=O)n2c1] | 1113 |
| 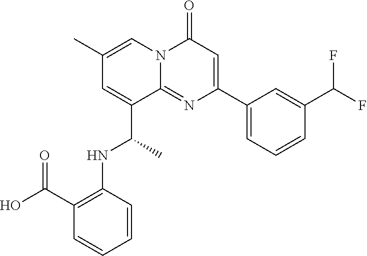 [SMILES: Cc1cc([C@H](C)Nc2ccccc2C(=O)O)c2nc(-c3cccc(C(F)F)c3)cc(=O)n2c1] | 1114 |
| 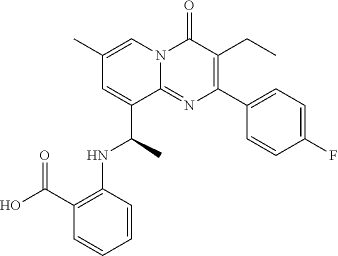 [SMILES: CCc1c(-c2ccc(F)cc2)nc2c([C@@H](C)Nc3ccccc3C(=O)O)cc(C)cn2c1=O] | 1115 |
| 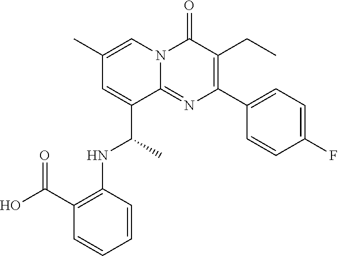 [SMILES: CCc1c(-c2ccc(F)cc2)nc2c([C@H](C)Nc3ccccc3C(=O)O)cc(C)cn2c1=O] | 1116 |
| 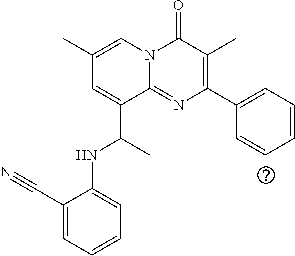 [SMILES: Cc1cc(C(C)Nc2ccccc2C#N)c2nc(-c3ccccc3)c(C)c(=O)n2c1] | 1117 |
| 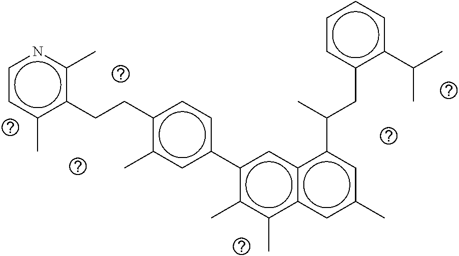 | 1118 |
| 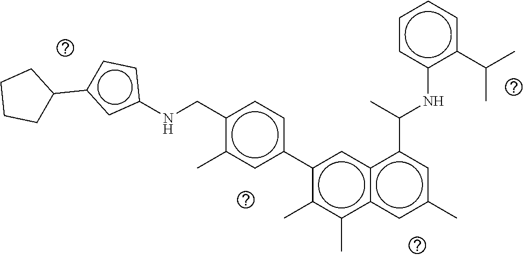 | 1119 |
| 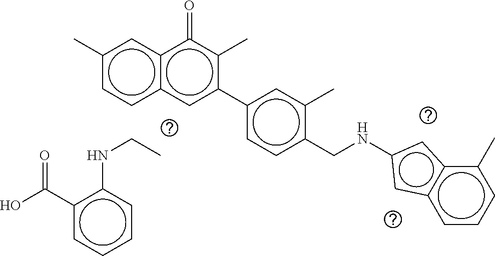 | 1120 |
| 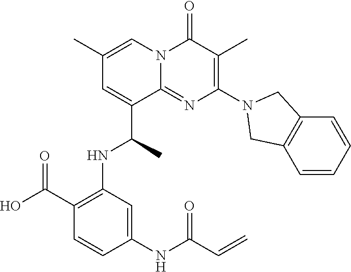 [SMILES: C=CC(=O)Nc1ccc(C(=O)O)c(N[C@H](C)c2cc(C)cn3c(=O)c(C)c(N4Cc5ccccc5C4)nc23)c1] | 1121 |
| 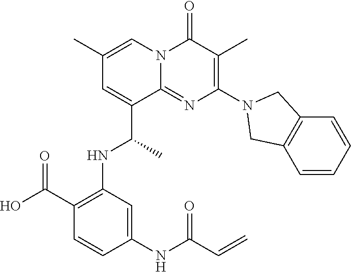 [SMILES: C=CC(=O)Nc1ccc(C(=O)O)c(N[C@@H](C)c2cc(C)cn3c(=O)c(C)c(N4Cc5ccccc5C4)nc23)c1] | 1122 |
| 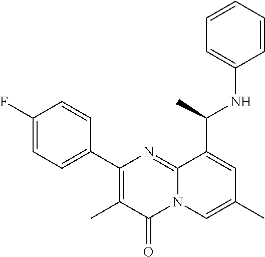 [SMILES: Cc1cc([C@@H](C)Nc2ccccc2)c2nc(-c3ccc(F)cc3)c(C)c(=O)n2c1] | 1123 |
| 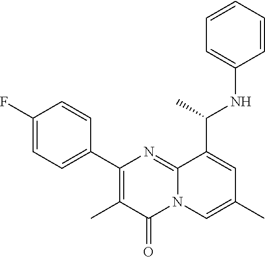 [SMILES: Cc1cc([C@H](C)Nc2ccccc2)c2nc(-c3ccc(F)cc3)c(C)c(=O)n2c1] | 1124 |
| 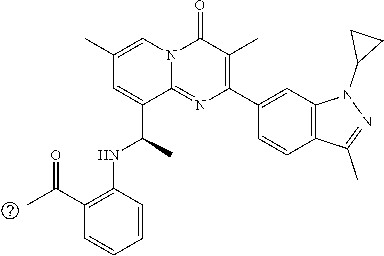 [SMILES: CC(=O)c1ccccc1N[C@H](C)c1cc(C)cn2c(=O)c(C)c(-c3ccc4c(C)nn(C5CC5)c4c3)nc12] | 1125 |
| 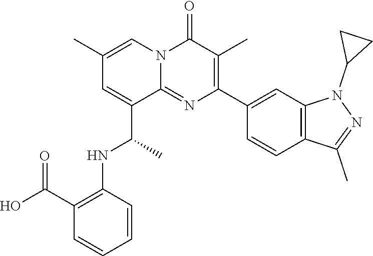 [SMILES: Cc1cc([C@H](C)Nc2ccccc2C(=O)O)c2nc(-c3ccc4c(C)nn(C5CC5)c4c3)c(C)c(=O)n2c1] | 1126 |
| 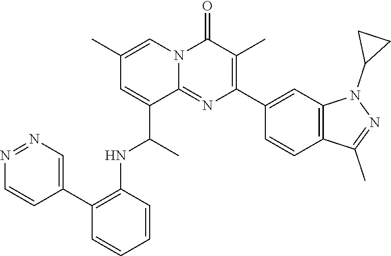 [SMILES: Cc1cc(C(C)Nc2ccccc2-c2ccnnc2)c2nc(-c3ccc4c(C)nn(C5CC5)c4c3)c(C)c(=O)n2c1] | 1127 |
| 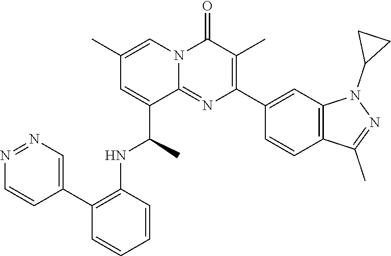 [SMILES: Cc1cc([C@@H](C)Nc2ccccc2-c2ccnnc2)c2nc(-c3ccc4c(C)nn(C5CC5)c4c3)c(C)c(=O)n2c1] | 1128 |
| 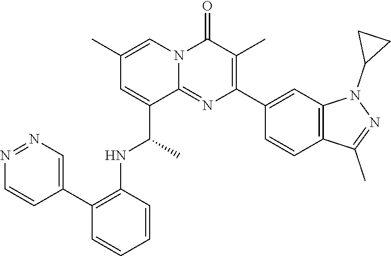 [SMILES: Cc1cc([C@H](C)Nc2ccccc2-c2ccnnc2)c2nc(-c3ccc4c(C)nn(C5CC5)c4c3)c(C)c(=O)n2c1] | 1129 |
| 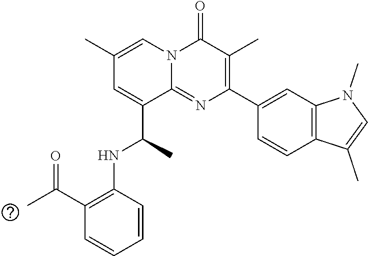 [SMILES: CC(=O)c1ccccc1N[C@H](C)c1cc(C)cn2c(=O)c(C)c(-c3ccc4c(C)cn(C)c4c3)nc12] | 1130 |
| 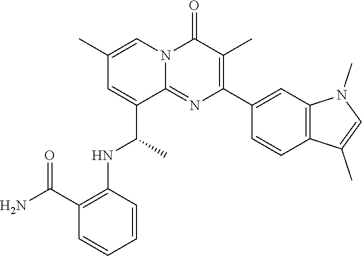 [SMILES: Cc1cc([C@H](C)Nc2ccccc2C(N)=O)c2nc(-c3ccc4c(C)cn(C)c4c3)c(C)c(=O)n2c1] | 1131 |
| 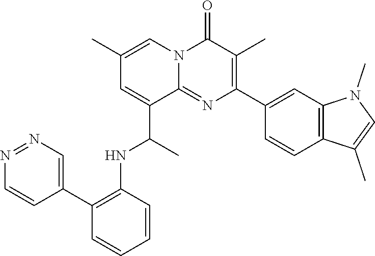 [SMILES: Cc1cc(C(C)Nc2ccccc2-c2ccnnc2)c2nc(-c3ccc4c(C)cn(C)c4c3)c(C)c(=O)n2c1] | 1132 |
| 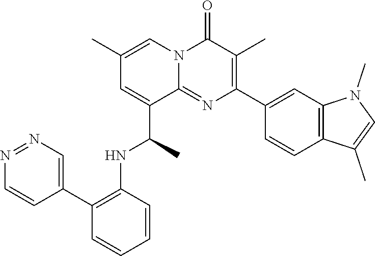 [SMILES: Cc1cc([C@@H](C)Nc2ccccc2-c2ccnnc2)c2nc(-c3ccc4c(C)cn(C)c4c3)c(C)c(=O)n2c1] | 1133 |
| 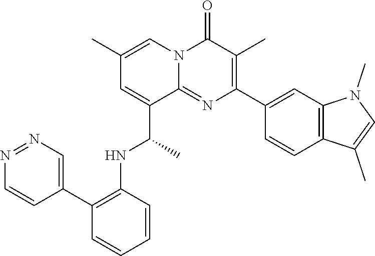 [SMILES: Cc1cc([C@H](C)Nc2ccccc2-c2ccnnc2)c2nc(-c3ccc4c(C)cn(C)c4c3)c(C)c(=O)n2c1] | 1134 |
| 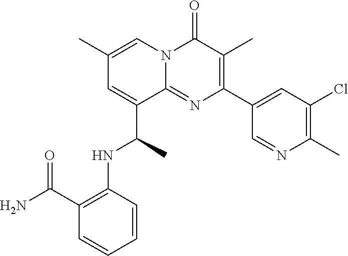 [SMILES: Cc1cc([C@@H](C)Nc2ccccc2C(N)=O)c2nc(-c3cnc(C)c(Cl)c3)c(C)c(=O)n2c1] | 1135 |
| 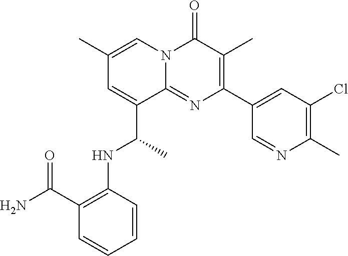 [SMILES: Cc1cc([C@H](C)Nc2ccccc2C(N)=O)c2nc(-c3cnc(C)c(Cl)c3)c(C)c(=O)n2c1] | 1136 |
| 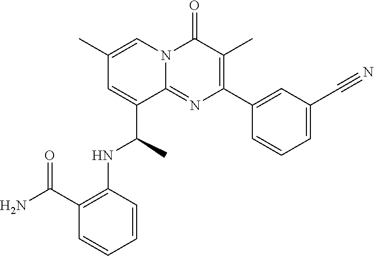 [SMILES: Cc1cc([C@@H](C)Nc2ccccc2C(N)=O)c2nc(-c3cccc(C#N)c3)c(C)c(=O)n2c1] | 1137 |
| 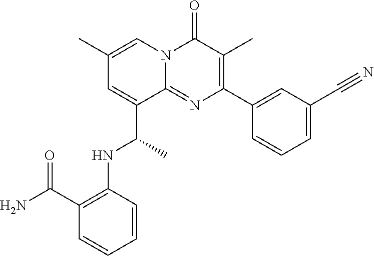 [SMILES: Cc1cc([C@H](C)Nc2ccccc2C(N)=O)c2nc(-c3cccc(C#N)c3)c(C)c(=O)n2c1] | 1138 |
| 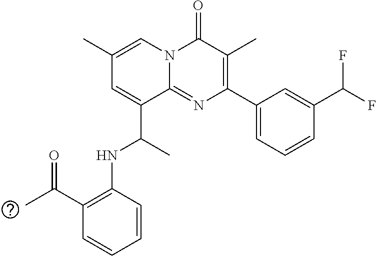 [SMILES: CC(=O)c1ccccc1NC(C)c1cc(C)cn2c(=O)c(C)c(-c3cccc(C(F)F)c3)nc12] | 1139 |
| 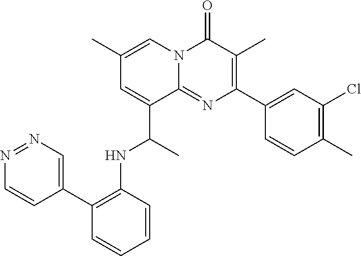 [SMILES: Cc1cc(C(C)Nc2ccccc2-c2ccnnc2)c2nc(-c3ccc(C)c(Cl)c3)c(C)c(=O)n2c1] | 1140 |
| 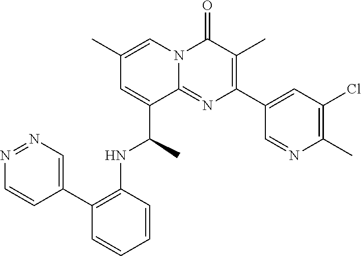 [SMILES: Cc1cc([C@@H](C)Nc2ccccc2-c2ccnnc2)c2nc(-c3cnc(C)c(Cl)c3)c(C)c(=O)n2c1] | 1141 |
| 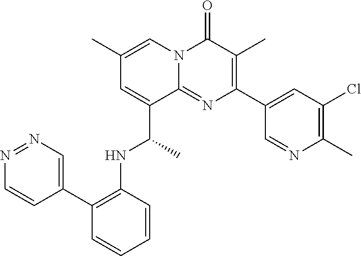 [SMILES: Cc1cc([C@H](C)Nc2ccccc2-c2ccnnc2)c2nc(-c3cnc(C)c(Cl)c3)c(C)c(=O)n2c1] | 1142 |
| 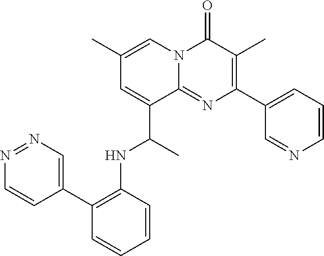 [SMILES: Cc1cc(C(C)Nc2ccccc2-c2ccnnc2)c2nc(-c3cccnc3)c(C)c(=O)n2c1] | 1143 |
| 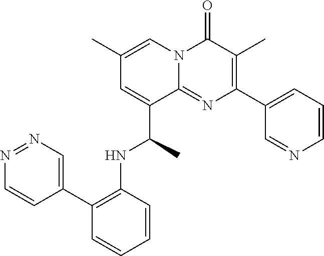 [SMILES: Cc1cc([C@@H](C)Nc2ccccc2-c2ccnnc2)c2nc(-c3cccnc3)c(C)c(=O)n2c1] | 1144 |
| 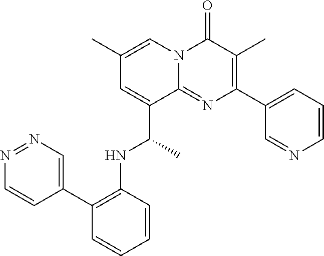 [SMILES: Cc1cc([C@H](C)Nc2ccccc2-c2ccnnc2)c2nc(-c3cccnc3)c(C)c(=O)n2c1] | 1145 |
| 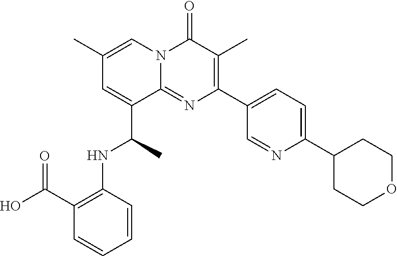 [SMILES: Cc1cc([C@@H](C)Nc2ccccc2C(=O)O)c2nc(-c3ccc(C4CCOCC4)nc3)c(C)c(=O)n2c1] | 1146 |
| 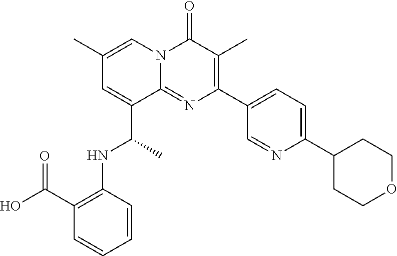 [SMILES: Cc1cc([C@H](C)Nc2ccccc2C(=O)O)c2nc(-c3ccc(C4CCOCC4)nc3)c(C)c(=O)n2c1] | 1147 |
| 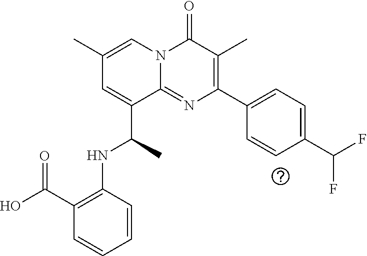 [SMILES: Cc1cc([C@@H](C)Nc2ccccc2C(=O)O)c2nc(-c3ccc(C(F)F)cc3)c(C)c(=O)n2c1] | 1148 |
| 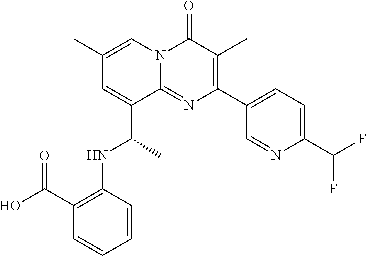 [SMILES: Cc1cc([C@H](C)Nc2ccccc2C(=O)O)c2nc(-c3ccc(C(F)F)nc3)c(C)c(=O)n2c1] | 1149 |
| 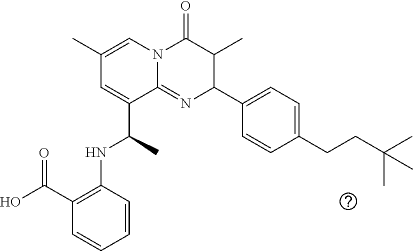 [SMILES: CC1=CN2C(=O)C(C)C(c3ccc(CCC(C)(C)C)cc3)N=C2C([C@@H](C)Nc2ccccc2C(=O)O)=C1] | 1150 |
| 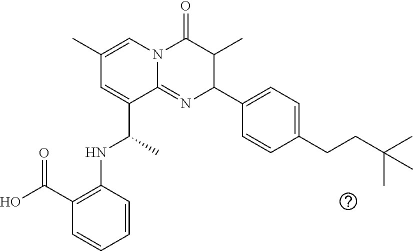 [SMILES: CC1=CN2C(=O)C(C)C(c3ccc(CCC(C)(C)C)cc3)N=C2C([C@H](C)Nc2ccccc2C(=O)O)=C1] | 1151 |
| 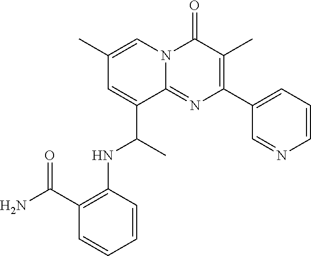 [SMILES: Cc1cc(C(C)Nc2ccccc2C(N)=O)c2nc(-c3cccnc3)c(C)c(=O)n2c1] | 1152 |
| 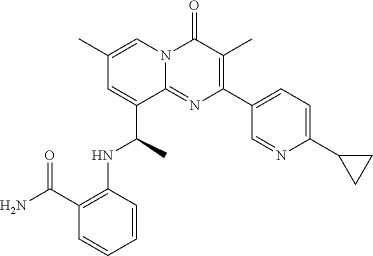 [SMILES: Cc1cc([C@@H](C)Nc2ccccc2C(N)=O)c2nc(-c3ccc(C4CC4)nc3)c(C)c(=O)n2c1] | 1153 |
| 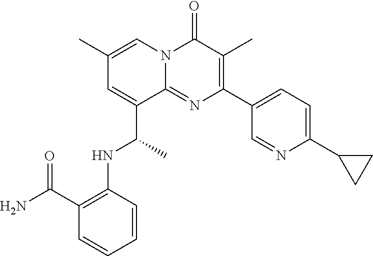 [SMILES: Cc1cc([C@H](C)Nc2ccccc2C(N)=O)c2nc(-c3ccc(C4CC4)nc3)c(C)c(=O)n2c1] | 1154 |
| 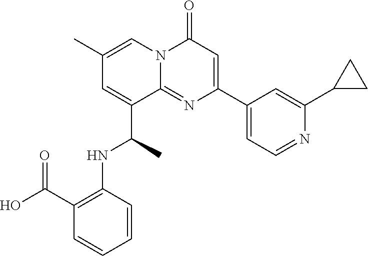 [SMILES: Cc1cc([C@@H](C)Nc2ccccc2C(=O)O)c2nc(-c3ccnc(C4CC4)c3)cc(=O)n2c1] | 1155 |
| 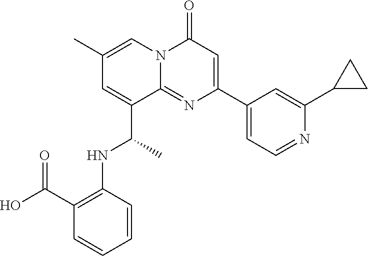 [SMILES: Cc1cc([C@H](C)Nc2ccccc2C(=O)O)c2nc(-c3ccnc(C4CC4)c3)cc(=O)n2c1] | 1156 |
| 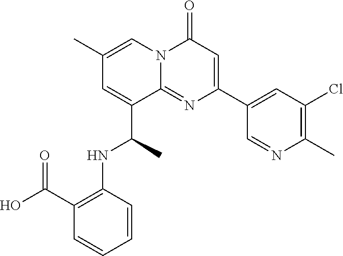 [SMILES: Cc1cc([C@@H](C)Nc2ccccc2C(=O)O)c2nc(-c3cnc(C)c(Cl)c3)cc(=O)n2c1] | 1157 |
| 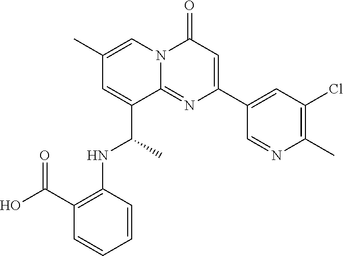 [SMILES: Cc1cc([C@H](C)Nc2ccccc2C(=O)O)c2nc(-c3cnc(C)c(Cl)c3)cc(=O)n2c1] | 1158 |
| 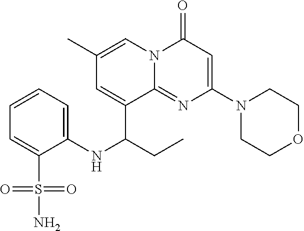 [SMILES: CCC(Nc1ccccc1S(N)(=O)=O)c1cc(C)cn2c(=O)cc(N3CCOCC3)nc12] | 1159 |
| 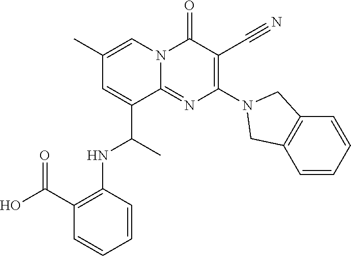 [SMILES: Cc1cc(C(C)Nc2ccccc2C(=O)O)c2nc(N3Cc4ccccc4C3)c(C#N)c(=O)n2c1] | 1160 |
| 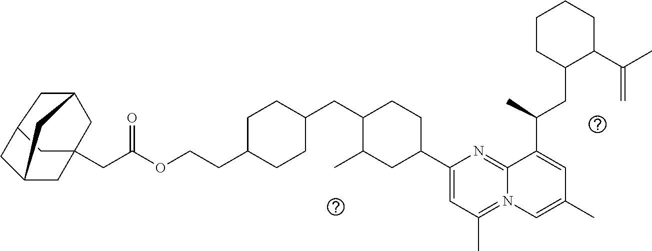 | 1161 |
| 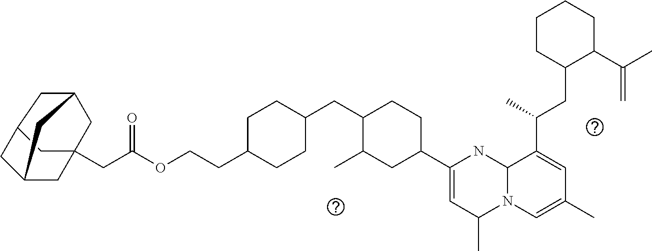 [SMILES: C=C(C)C1CCCCC1C[C@@H](C)C1=CC(C)=CN2C(C)C=C(C3CCC(CC4CCC(CCOC(=O)CC56CC7C[C@H](C5)C[C@@H](C7)C6)CC4)C(C)C3)NC12] | 1162 |
| 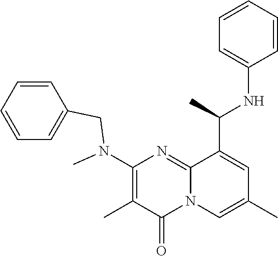 [SMILES: Cc1cc([C@@H](C)Nc2ccccc2)c2nc(N(C)Cc3ccccc3)c(C)c(=O)n2c1] | 1163 |
| 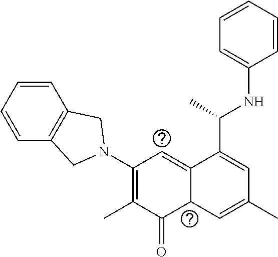 [SMILES: CC1=CC2C(=O)C(C)=C(N3Cc4ccccc4C3)C=C2C([C@H](C)Nc2ccccc2)=C1] | 1164 |
| 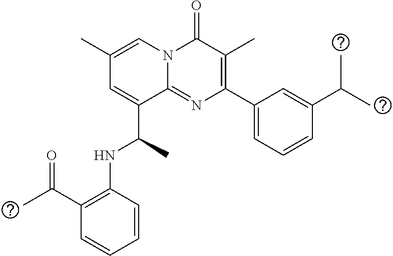 [SMILES: CC(=O)c1ccccc1N[C@H](C)c1cc(C)cn2c(=O)c(C)c(-c3cccc(C(C)C)c3)nc12] | 1165 |
| 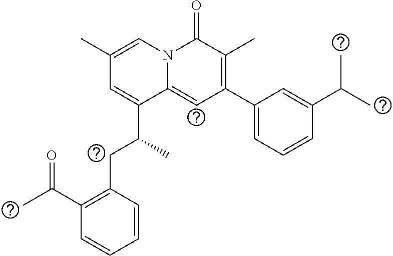 [SMILES: CC(=O)c1ccccc1C[C@@H](C)c1cc(C)cn2c(=O)c(C)c(-c3cccc(C(C)C)c3)cc12] | 1166 |
| 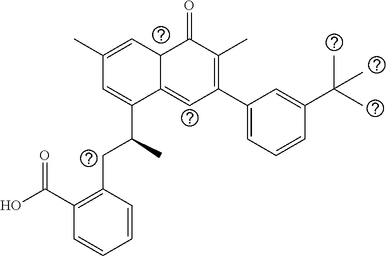 [SMILES: CC1=CC2C(=O)C(C)=C(c3cccc(C(C)(C)C)c3)C=C2C([C@@H](C)Cc2ccccc2C(=O)O)=C1] | 1167 |
| 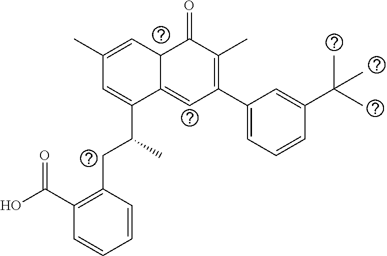 [SMILES: CC1=CC2C(=O)C(C)=C(c3cccc(C(C)(C)C)c3)C=C2C([C@H](C)Cc2ccccc2C(=O)O)=C1] | 1168 |
| 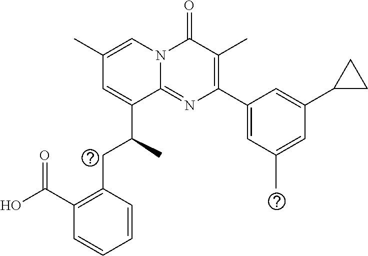 [SMILES: Cc1cc(-c2nc3c([C@@H](C)Cc4ccccc4C(=O)O)cc(C)cn3c(=O)c2C)cc(C2CC2)c1] | 1169 |
| 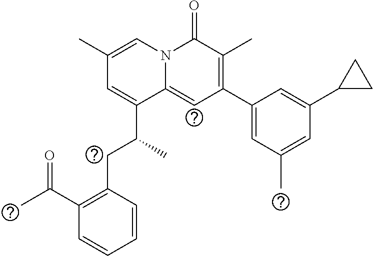 [SMILES: CC(=O)c1ccccc1C[C@@H](C)c1cc(C)cn2c(=O)c(C)c(-c3cc(C)cc(C4CC4)c3)cc12] | 1170 |
| 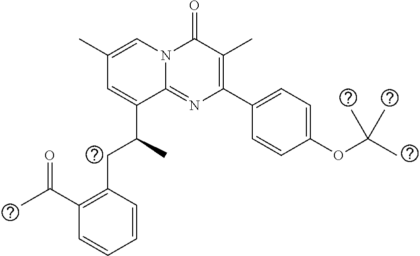 [SMILES: CC(=O)c1ccccc1C[C@H](C)c1cc(C)cn2c(=O)c(C)c(-c3ccc(OC(C)(C)C)cc3)nc12] | 1171 |
| 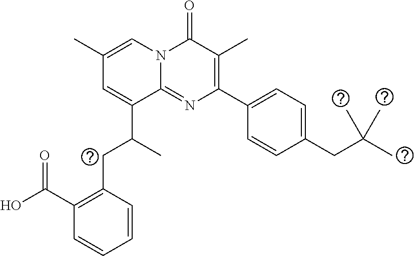 [SMILES: Cc1cc(C(C)Cc2ccccc2C(=O)O)c2nc(-c3ccc(CC(C)(C)C)cc3)c(C)c(=O)n2c1] | 1172 |
| 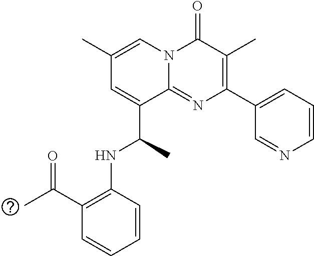 [SMILES: CC(=O)c1ccccc1N[C@H](C)c1cc(C)cn2c(=O)c(C)c(-c3cccnc3)nc12] | 1173 |
| 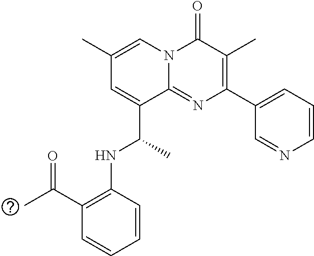 [SMILES: CC(=O)c1ccccc1N[C@@H](C)c1cc(C)cn2c(=O)c(C)c(-c3cccnc3)nc12] | 1174 |
| 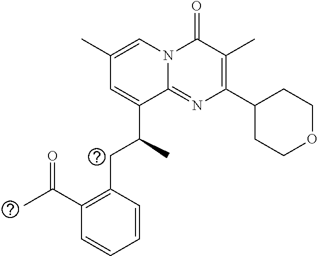 [SMILES: CC(=O)c1ccccc1C[C@H](C)c1cc(C)cn2c(=O)c(C)c(C3CCOCC3)nc12] | 1175 |
| 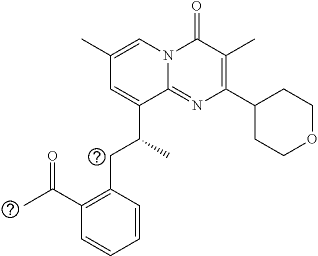 [SMILES: CC(=O)c1ccccc1C[C@@H](C)c1cc(C)cn2c(=O)c(C)c(C3CCOCC3)nc12] | 1176 |
| 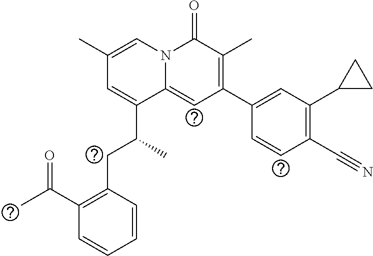 [SMILES: CC(=O)c1ccccc1C[C@@H](C)c1cc(C)cn2c(=O)c(C)c(-c3ccc(C#N)c(C4CC4)c3)cc12] | 1177 |
| 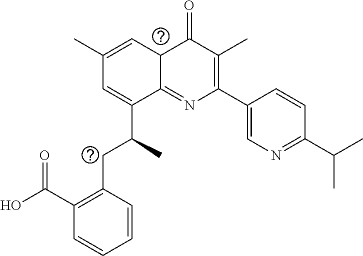 [SMILES: CC1=CC2C(=O)C(C)=C(c3ccc(C(C)C)nc3)N=C2C([C@@H](C)Cc2ccccc2C(=O)O)=C1] | 1178 |
| 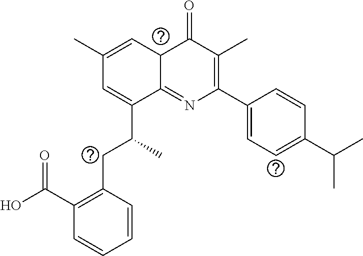 [SMILES: CC1=CC2C(=O)C(C)=C(c3ccc(C(C)C)cc3)N=C2C([C@H](C)Cc2ccccc2C(=O)O)=C1] | 1179 |
| 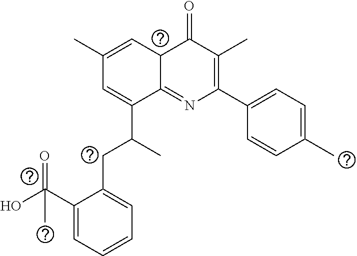 | 1180 |
| 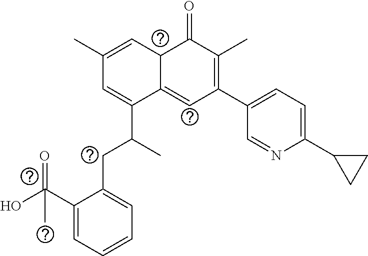 | 1181 |
| 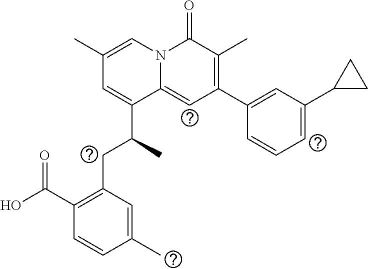 [SMILES: Cc1ccc(C(=O)O)c(C[C@H](C)c2cc(C)cn3c(=O)c(C)c(-c4cccc(C5CC5)c4)cc23)c1] | 1182 |
| 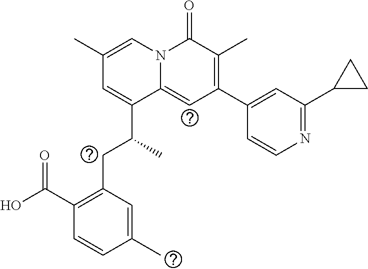 [SMILES: Cc1ccc(C(=O)O)c(C[C@@H](C)c2cc(C)cn3c(=O)c(C)c(-c4ccnc(C5CC5)c4)cc23)c1] | 1183 |
| 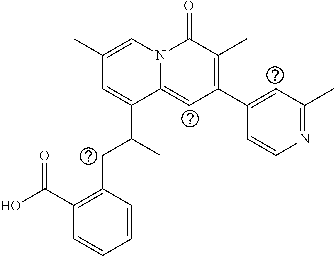 [SMILES: Cc1cc(C(C)Cc2ccccc2C(=O)O)c2cc(-c3ccnc(C)c3)c(C)c(=O)n2c1] | 1184 |
| 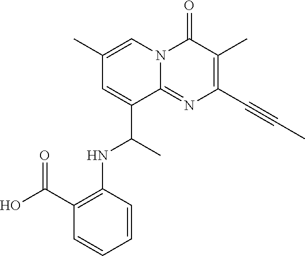 [SMILES: CC#Cc1nc2c(C(C)Nc3ccccc3C(=O)O)cc(C)cn2c(=O)c1C] | 1185 |
| 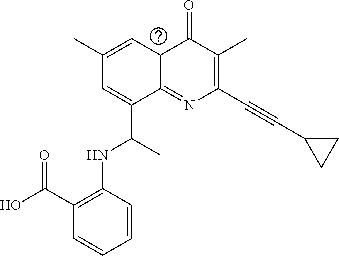 [SMILES: CC1=CC2C(=O)C(C)=C(C#CC3CC3)N=C2C(C(C)Nc2ccccc2C(=O)O)=C1] | 1186 |
| 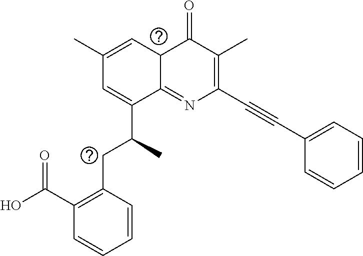 [SMILES: CC1=CC2C(=O)C(C)=C(C#Cc3ccccc3)N=C2C([C@@H](C)Cc2ccccc2C(=O)O)=C1] | 1187 |
| 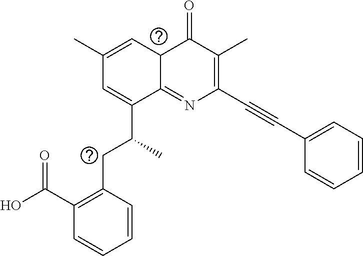 [SMILES: CC1=CC2C(=O)C(C)=C(C#Cc3ccccc3)N=C2C([C@H](C)Cc2ccccc2C(=O)O)=C1] | 1188 |
| 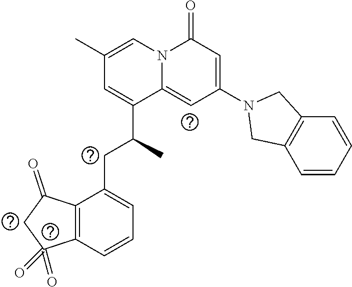 | 1189 |
| 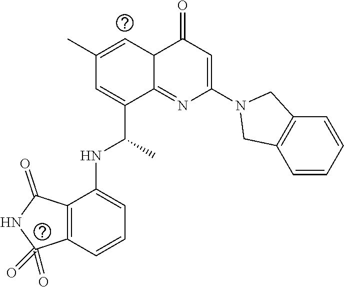 | 1190 |
| 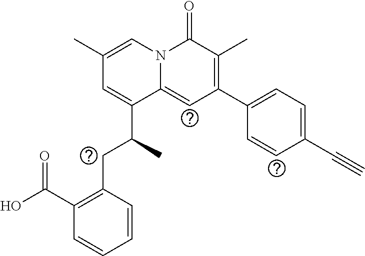 [SMILES: C#Cc1ccc(-c2cc3c([C@@H](C)Cc4ccccc4C(=O)O)cc(C)cn3c(=O)c2C)cc1] | 1191 |
| 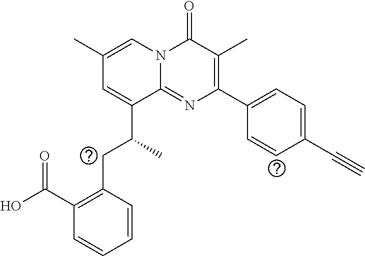 [SMILES: C#Cc1ccc(-c2nc3c([C@H](C)Cc4ccccc4C(=O)O)cc(C)cn3c(=O)c2C)cc1] | 1192 |
| 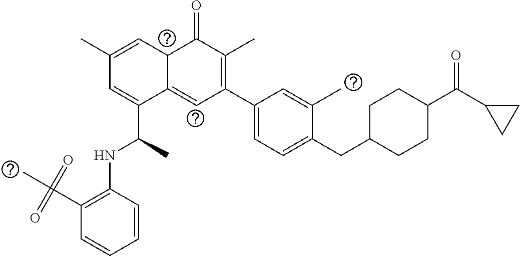 | 1193 |
| 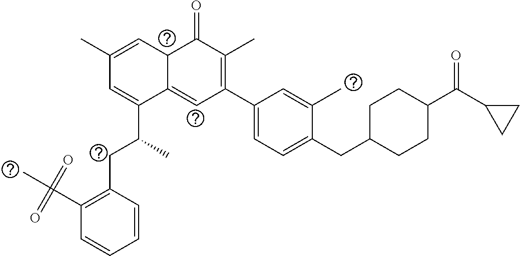 | 1194 |
| 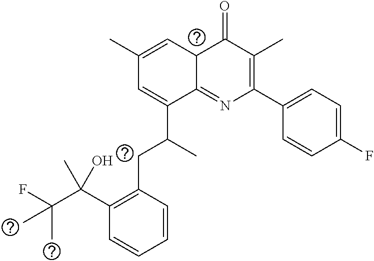 [SMILES: CC1=CC2C(=O)C(C)=C(c3ccc(F)cc3)N=C2C(C(C)Cc2ccccc2C(C)(O)C(C)(C)F)=C1] | 1195 |
| 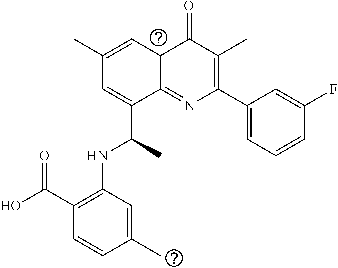 [SMILES: CC1=CC2C(=O)C(C)=C(c3cccc(F)c3)N=C2C([C@@H](C)Nc2cc(C)ccc2C(=O)O)=C1] | 1196 |
| 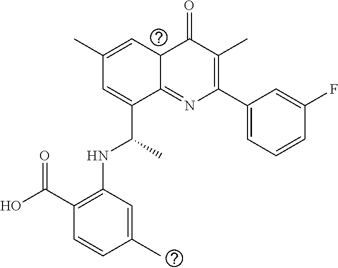 [SMILES: CC1=CC2C(=O)C(C)=C(c3cccc(F)c3)N=C2C([C@H](C)Nc2cc(C)ccc2C(=O)O)=C1] | 1197 |
| 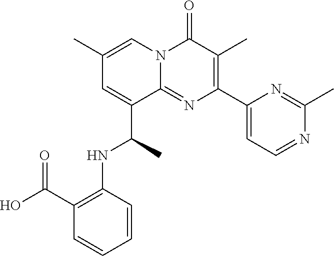 [SMILES: Cc1cc([C@@H](C)Nc2ccccc2C(=O)O)c2nc(-c3ccnc(C)n3)c(C)c(=O)n2c1] | 1198 |
| 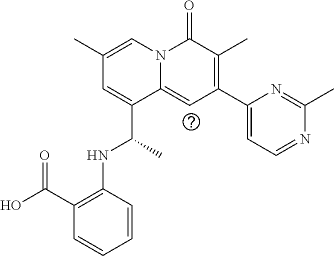 [SMILES: Cc1cc([C@H](C)Nc2ccccc2C(=O)O)c2cc(-c3ccnc(C)n3)c(C)c(=O)n2c1] | 1199 |
| 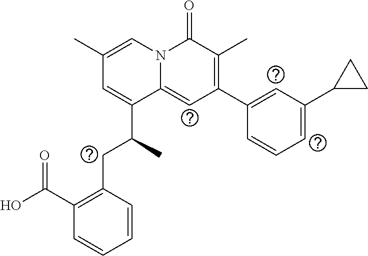 [SMILES: Cc1cc([C@@H](C)Cc2ccccc2C(=O)O)c2cc(-c3cccc(C4CC4)c3)c(C)c(=O)n2c1] | 1200 |
| 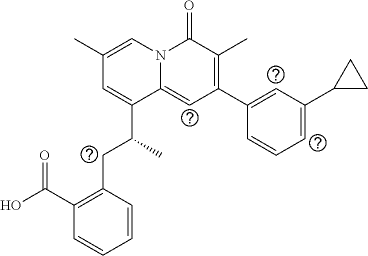 [SMILES: Cc1cc([C@H](C)Cc2ccccc2C(=O)O)c2cc(-c3cccc(C4CC4)c3)c(C)c(=O)n2c1] | 1201 |
| 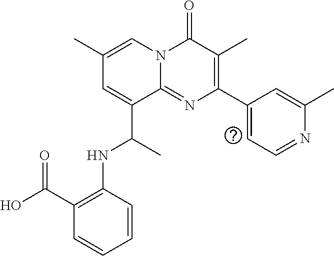 [SMILES: Cc1cc(C(C)Nc2ccccc2C(=O)O)c2nc(-c3ccnc(C)c3)c(C)c(=O)n2c1] | 1202 |
| 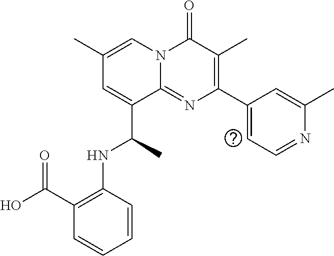 [SMILES: Cc1cc([C@@H](C)Nc2ccccc2C(=O)O)c2nc(-c3ccnc(C)c3)c(C)c(=O)n2c1] | 1203 |
| 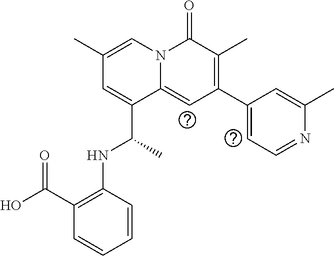 [SMILES: Cc1cc([C@H](C)Nc2ccccc2C(=O)O)c2cc(-c3ccnc(C)c3)c(C)c(=O)n2c1] | 1204 |
| 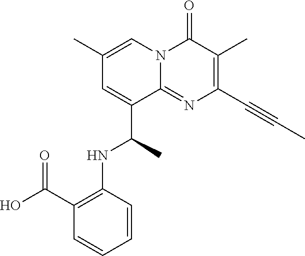 [SMILES: CC#Cc1nc2c([C@@H](C)Nc3ccccc3C(=O)O)cc(C)cn2c(=O)c1C] | 1205 |
| 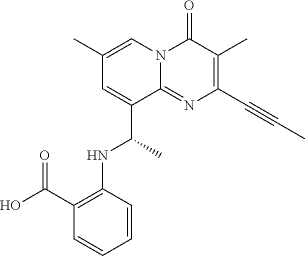 [SMILES: CC#Cc1nc2c([C@H](C)Nc3ccccc3C(=O)O)cc(C)cn2c(=O)c1C] | 1206 |
| 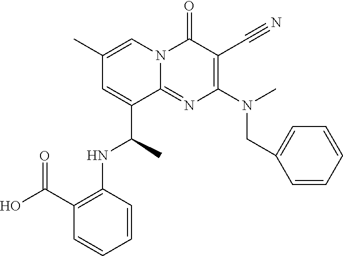 [SMILES: Cc1cc([C@@H](C)Nc2ccccc2C(=O)O)c2nc(N(C)Cc3ccccc3)c(C#N)c(=O)n2c1] | 1207 |
| 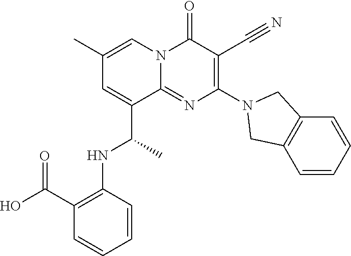 [SMILES: Cc1cc([C@H](C)Nc2ccccc2C(=O)O)c2nc(N3Cc4ccccc4C3)c(C#N)c(=O)n2c1] | 1208 |
| 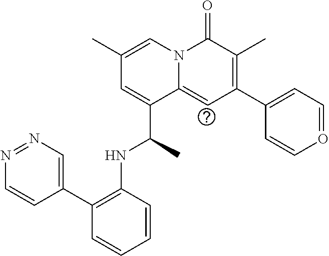 | 1209 |
| 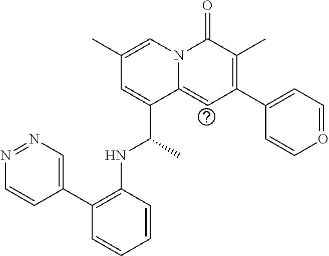 | 1210 |
| 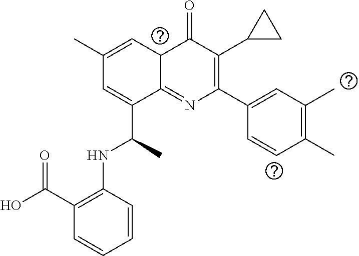 [SMILES: CC1=CC2C(=O)C(C3CC3)=C(c3ccc(C)c(C)c3)N=C2C([C@@H](C)Nc2ccccc2C(=O)O)=C1] | 1211 |
| 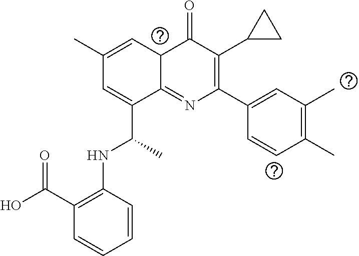 [SMILES: CC1=CC2C(=O)C(C3CC3)=C(c3ccc(C)c(C)c3)N=C2C([C@H](C)Nc2ccccc2C(=O)O)=C1] | 1212 |
| 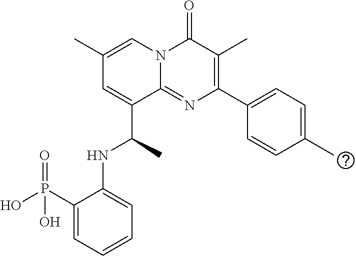 [SMILES: Cc1ccc(-c2nc3c([C@@H](C)Nc4ccccc4P(=O)(O)O)cc(C)cn3c(=O)c2C)cc1] | 1213 |
| 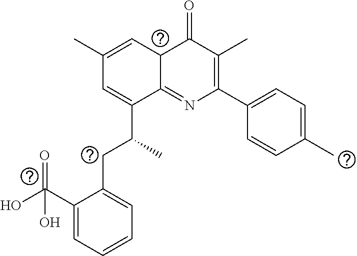 | 1214 |
| 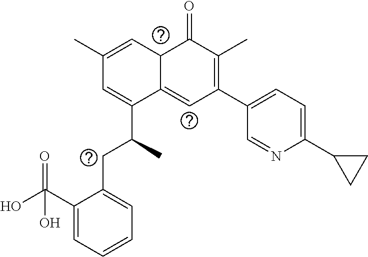 | 1215 |
| 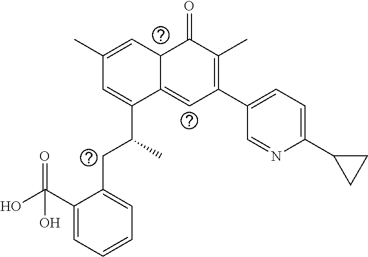 | 1216 |
| 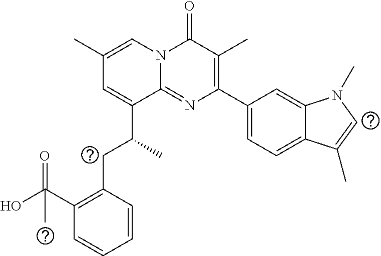 | 1217 |
| 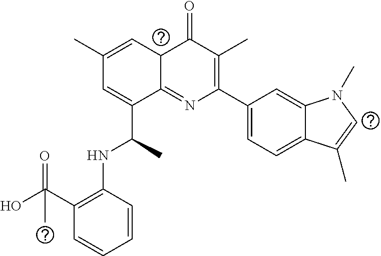 | 1218 |
| 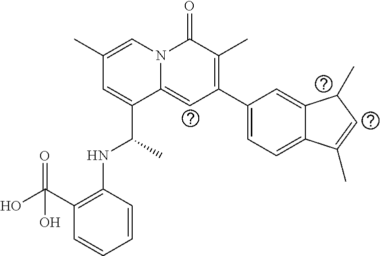 | 1219 |
| 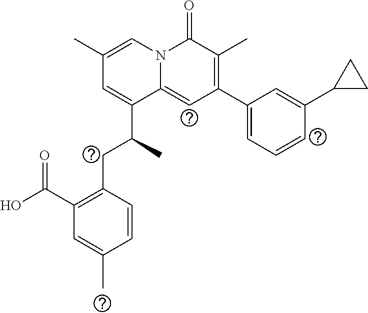 [SMILES: Cc1ccc(C[C@H](C)c2cc(C)cn3c(=O)c(C)c(-c4cccc(C5CC5)c4)cc23)c(C(=O)O)c1] | 1220 |
| 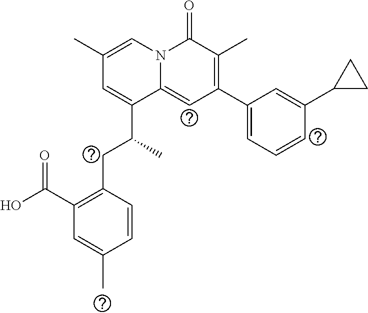 [SMILES: Cc1ccc(C[C@@H](C)c2cc(C)cn3c(=O)c(C)c(-c4cccc(C5CC5)c4)cc23)c(C(=O)O)c1] | 1221 |
| 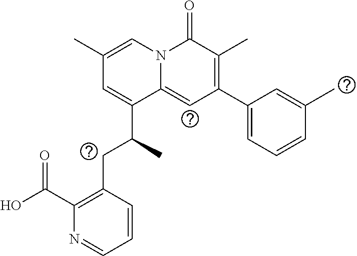 [SMILES: Cc1cccc(-c2cc3c([C@@H](C)Cc4cccnc4C(=O)O)cc(C)cn3c(=O)c2C)c1] | 1222 |
| 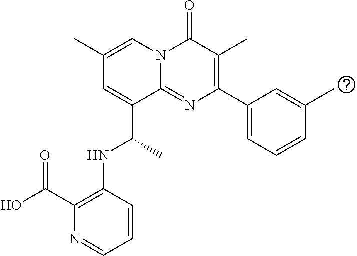 [SMILES: Cc1cccc(-c2nc3c([C@H](C)Nc4cccnc4C(=O)O)cc(C)cn3c(=O)c2C)c1] | 1223 |
| 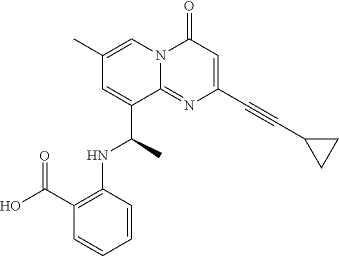 [SMILES: Cc1cc([C@@H](C)Nc2ccccc2C(=O)O)c2nc(C#CC3CC3)cc(=O)n2c1] | 1224 |
| 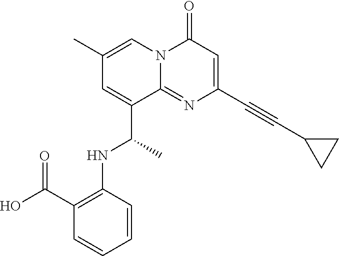 [SMILES: Cc1cc([C@H](C)Nc2ccccc2C(=O)O)c2nc(C#CC3CC3)cc(=O)n2c1] | 1225 |
| 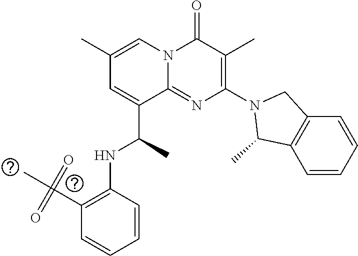 | 1226 |
| 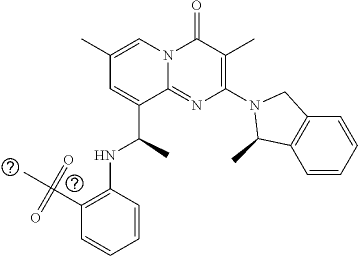 | 1227 |
| 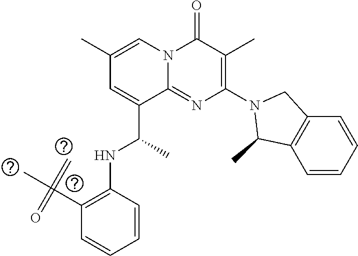 | 1228 |
| 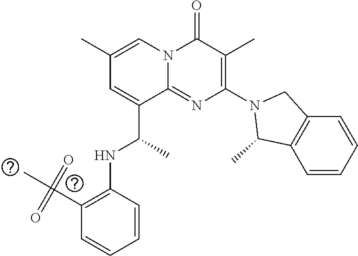 | 1229 |
| 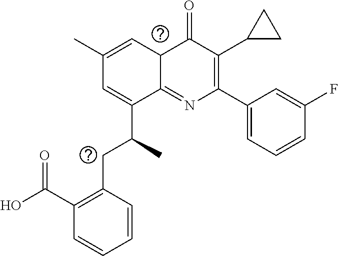 [SMILES: CC1=CC2C(=O)C(C3CC3)=C(c3cccc(F)c3)N=C2C([C@@H](C)Cc2ccccc2C(=O)O)=C1] | 1230 |
| 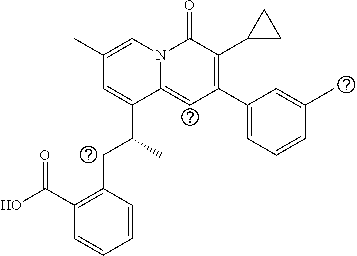 [SMILES: Cc1cccc(-c2cc3c([C@H](C)Cc4ccccc4C(=O)O)cc(C)cn3c(=O)c2C2CC2)c1] | 1231 |
| 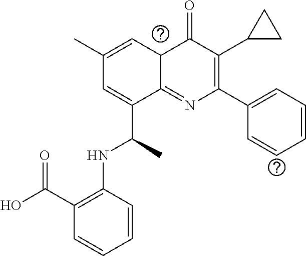 [SMILES: CC1=CC2C(=O)C(C3CC3)=C(c3ccccc3)N=C2C([C@@H](C)Nc2ccccc2C(=O)O)=C1] | 1232 |
| 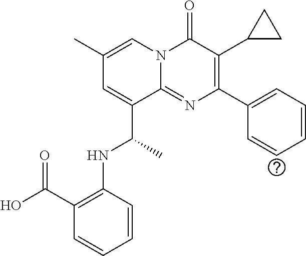 [SMILES: Cc1cc([C@H](C)Nc2ccccc2C(=O)O)c2nc(-c3ccccc3)c(C3CC3)c(=O)n2c1] | 1233 |
| 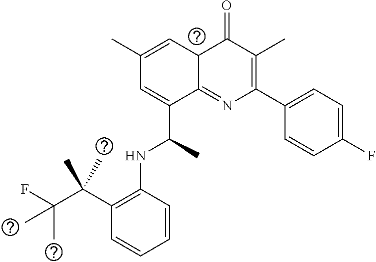 | 1234 |
| 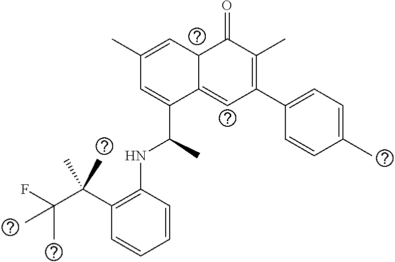 | 1235 |
| 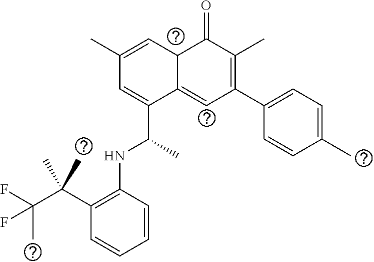 | 1236 |
| 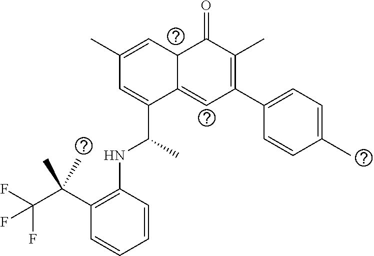 | 1237 |
| 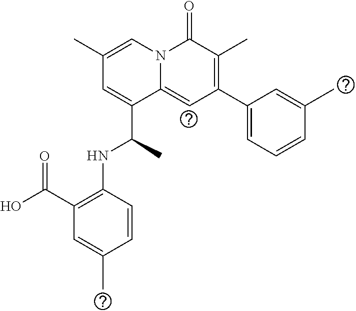 [SMILES: Cc1cccc(-c2cc3c([C@@H](C)Nc4ccc(C)cc4C(=O)O)cc(C)cn3c(=O)c2C)c1] | 1238 |
| 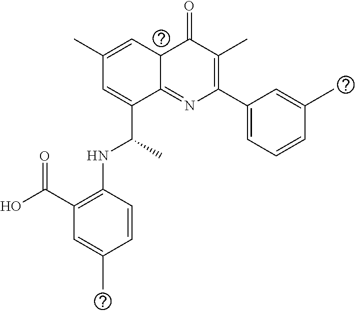 [SMILES: CC1=CC2C(=O)C(C)=C(c3cccc(C)c3)N=C2C([C@H](C)Nc2ccc(C)cc2C(=O)O)=C1] | 1239 |
| 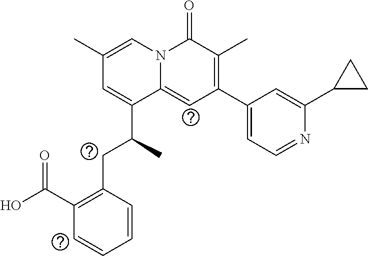 [SMILES: Cc1cc([C@@H](C)Cc2ccccc2C(=O)O)c2cc(-c3ccnc(C4CC4)c3)c(C)c(=O)n2c1] | 1240 |
| 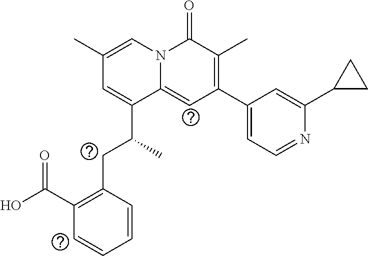 [SMILES: Cc1cc([C@H](C)Cc2ccccc2C(=O)O)c2cc(-c3ccnc(C4CC4)c3)c(C)c(=O)n2c1] | 1241 |
| 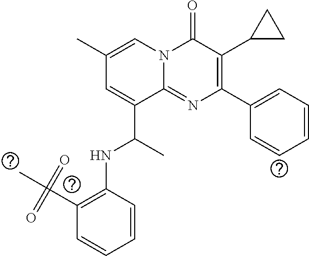 | 1242 |
| 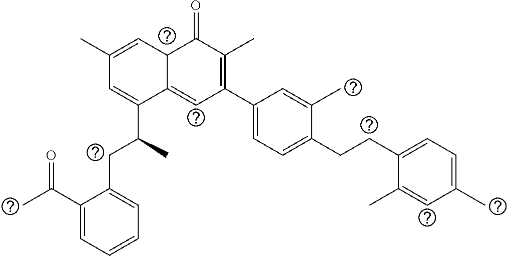 [SMILES: CC(=O)c1ccccc1C[C@H](C)C1=CC(C)=CC2C(=O)C(C)=C(c3ccc(CCc4ccc(C)cc4C)c(C)c3)C=C12] | 1243 |
| 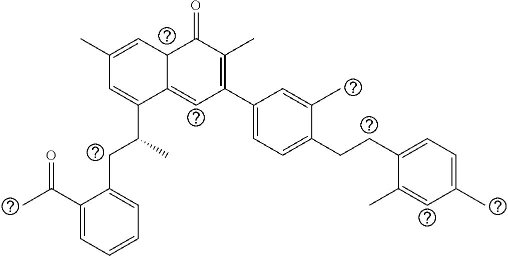 [SMILES: CC(=O)c1ccccc1C[C@@H](C)C1=CC(C)=CC2C(=O)C(C)=C(c3ccc(CCc4ccc(C)cc4C)c(C)c3)C=C12] | 1244 |
| 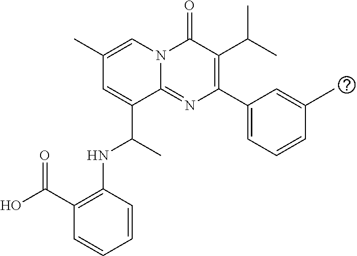 [SMILES: Cc1cccc(-c2nc3c(C(C)Nc4ccccc4C(=O)O)cc(C)cn3c(=O)c2C(C)C)c1] | 1245 |
| 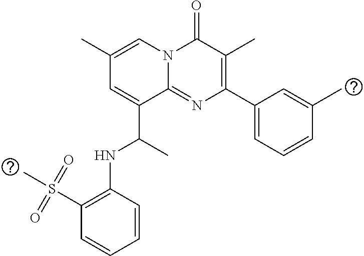 | 1246 |
| 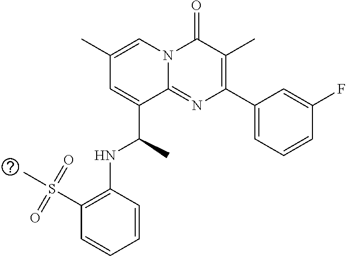 [SMILES: Cc1cc([C@@H](C)Nc2ccccc2S(C)(=O)=O)c2nc(-c3cccc(F)c3)c(C)c(=O)n2c1] | 1247 |
| 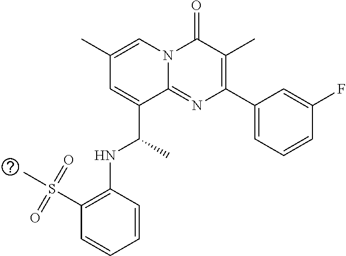 [SMILES: Cc1cc([C@H](C)Nc2ccccc2S(C)(=O)=O)c2nc(-c3cccc(F)c3)c(C)c(=O)n2c1] | 1248 |
| 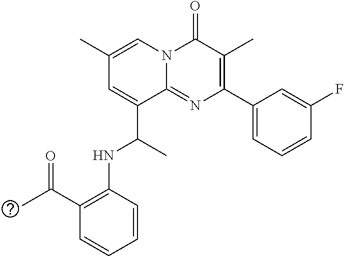 [SMILES: CC(=O)c1ccccc1NC(C)c1cc(C)cn2c(=O)c(C)c(-c3cccc(F)c3)nc12] | 1249 |
| 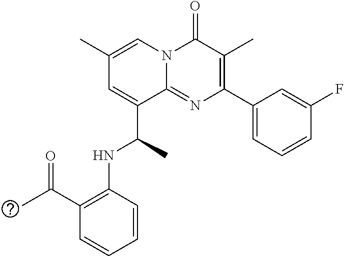 [SMILES: CC(=O)c1ccccc1N[C@H](C)c1cc(C)cn2c(=O)c(C)c(-c3cccc(F)c3)nc12] | 1250 |
| 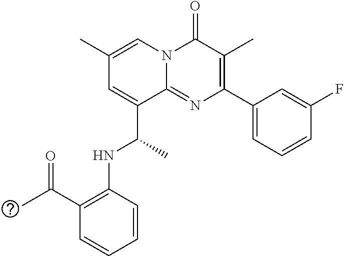 [SMILES: CC(=O)c1ccccc1N[C@@H](C)c1cc(C)cn2c(=O)c(C)c(-c3cccc(F)c3)nc12] | 1251 |
| 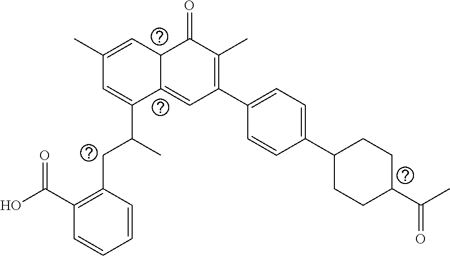 [SMILES: CC(=O)C1CCC(c2ccc(C3=C(C)C(=O)C4C=C(C)C=C(C(C)Cc5ccccc5C(=O)O)C4=C3)cc2)CC1] | 1252 |
| 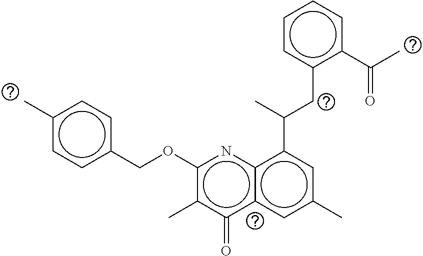 | 1253 |
| 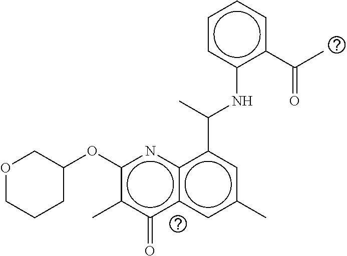 | 1254 |
| 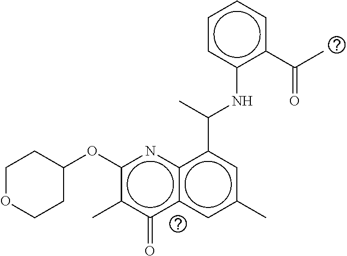 | 1255 |
| 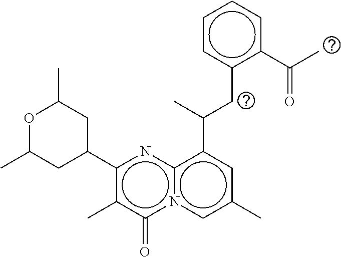 | 1256 |
| 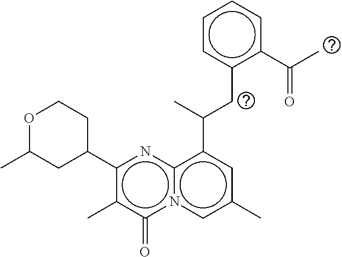 | 1257 |
| 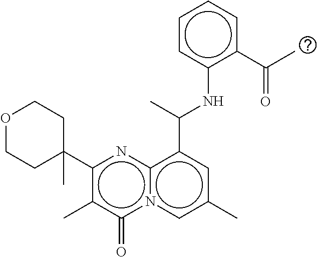 | 1258 |
| 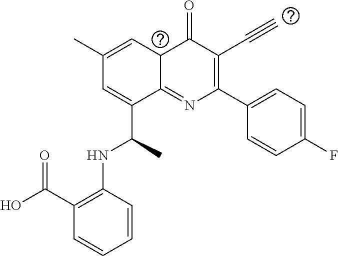 [SMILES: C#CC1=C(c2ccc(F)cc2)N=C2C([C@@H](C)Nc3ccccc3C(=O)O)=CC(C)=CC2C1=O] | 1259 |
| 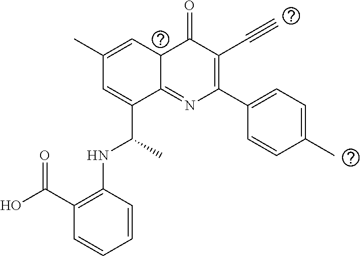 [SMILES: C#CC1=C(c2ccc(C)cc2)N=C2C([C@H](C)Nc3ccccc3C(=O)O)=CC(C)=CC2C1=O] | 1260 |
| 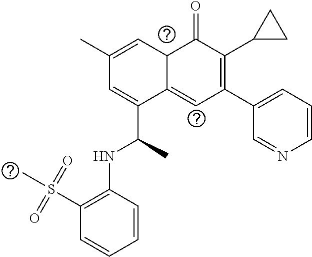 [SMILES: CC1=CC2C(=O)C(C3CC3)=C(c3cccnc3)C=C2C([C@@H](C)Nc2ccccc2S(C)(=O)=O)=C1] | 1261 |
| 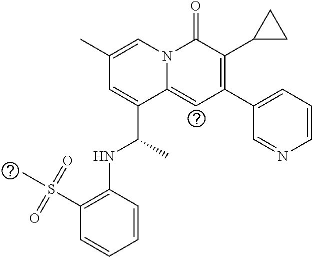 [SMILES: Cc1cc([C@H](C)Nc2ccccc2S(C)(=O)=O)c2cc(-c3cccnc3)c(C3CC3)c(=O)n2c1] | 1262 |
| 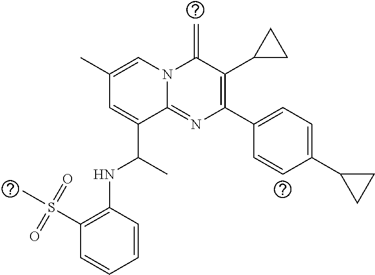 [SMILES: C=C1C(C2CC2)=C(c2ccc(C3CC3)cc2)N=C2C(C(C)Nc3ccccc3S(C)(=O)=O)=CC(C)=CN12] | 1263 |
| 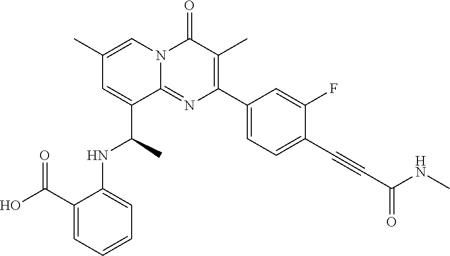 [SMILES: CNC(=O)C#Cc1ccc(-c2nc3c([C@@H](C)Nc4ccccc4C(=O)O)cc(C)cn3c(=O)c2C)cc1F] | 1264 |
| 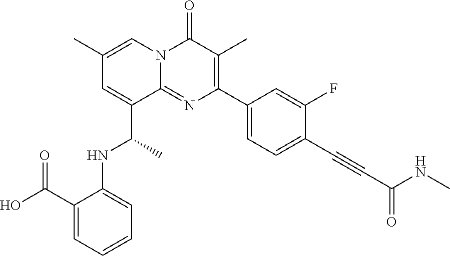 [SMILES: CNC(=O)C#Cc1ccc(-c2nc3c([C@H](C)Nc4ccccc4C(=O)O)cc(C)cn3c(=O)c2C)cc1F] | 1265 |
| 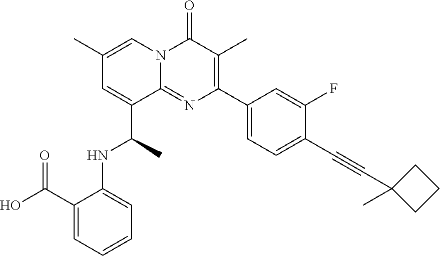 [SMILES: Cc1cc([C@@H](C)Nc2ccccc2C(=O)O)c2nc(-c3ccc(C#CC4(C)CCC4)c(F)c3)c(C)c(=O)n2c1] | 1266 |
| 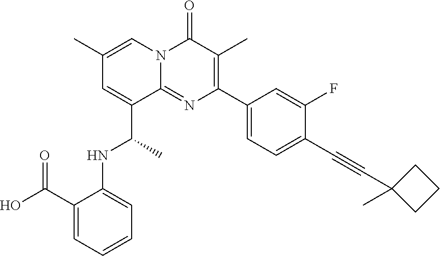 [SMILES: Cc1cc([C@H](C)Nc2ccccc2C(=O)O)c2nc(-c3ccc(C#CC4(C)CCC4)c(F)c3)c(C)c(=O)n2c1] | 1267 |
| 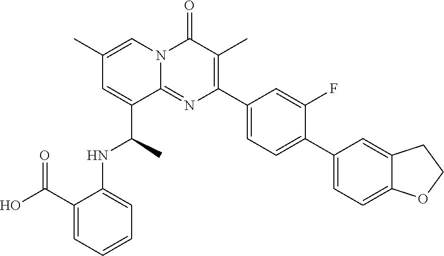 [SMILES: Cc1cc([C@@H](C)Nc2ccccc2C(=O)O)c2nc(-c3ccc(-c4ccc5c(c4)CCO5)c(F)c3)c(C)c(=O)n2c1] | 1268 |
| 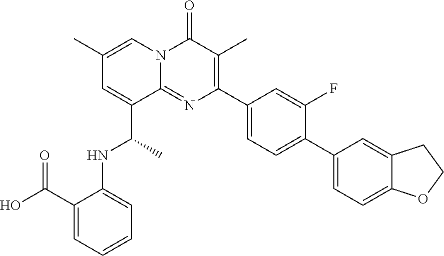 [SMILES: Cc1cc([C@H](C)Nc2ccccc2C(=O)O)c2nc(-c3ccc(-c4ccc5c(c4)CCO5)c(F)c3)c(C)c(=O)n2c1] | 1269 |
| 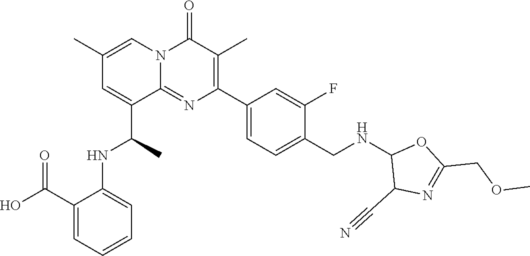 [SMILES: COCC1=NC(C#N)C(NCc2ccc(-c3nc4c([C@@H](C)Nc5ccccc5C(=O)O)cc(C)cn4c(=O)c3C)cc2F)O1] | 1270 |
| 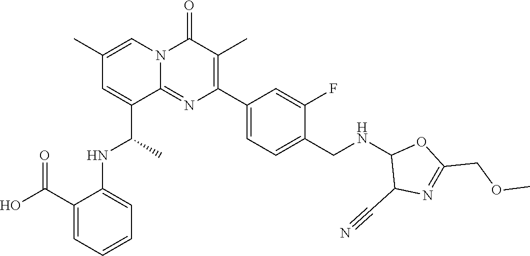 [SMILES: COCC1=NC(C#N)C(NCc2ccc(-c3nc4c([C@H](C)Nc5ccccc5C(=O)O)cc(C)cn4c(=O)c3C)cc2F)O1] | 1271 |
| 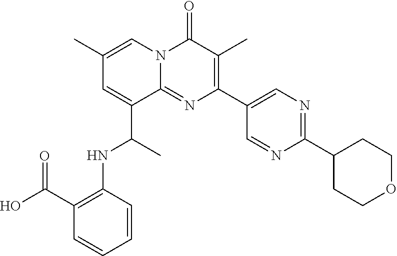 [SMILES: Cc1cc(C(C)Nc2ccccc2C(=O)O)c2nc(-c3cnc(C4CCOCC4)nc3)c(C)c(=O)n2c1] | 1272 |
| 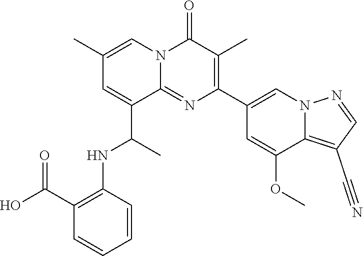 [SMILES: COc1cc(-c2nc3c(C(C)Nc4ccccc4C(=O)O)cc(C)cn3c(=O)c2C)cn2ncc(C#N)c12] | 1273 |
| 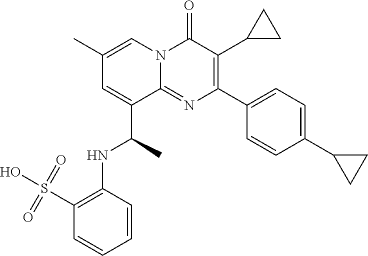 [SMILES: Cc1cc([C@@H](C)Nc2ccccc2S(=O)(=O)O)c2nc(-c3ccc(C4CC4)cc3)c(C3CC3)c(=O)n2c1] | 1274 |
| 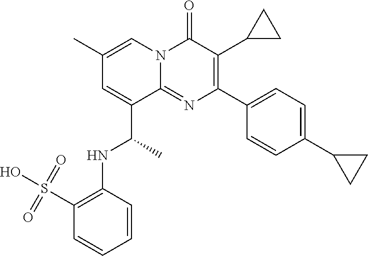 [SMILES: Cc1cc([C@H](C)Nc2ccccc2S(=O)(=O)O)c2nc(-c3ccc(C4CC4)cc3)c(C3CC3)c(=O)n2c1] | 1275 |
| 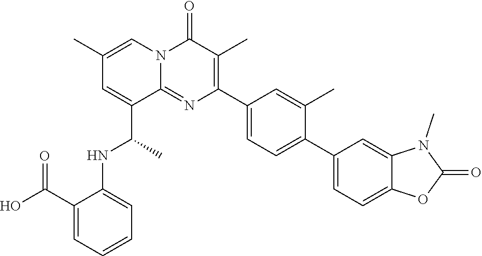 [SMILES: Cc1cc([C@H](C)Nc2ccccc2C(=O)O)c2nc(-c3ccc(-c4ccc5oc(=O)n(C)c5c4)c(C)c3)c(C)c(=O)n2c1] | 1276 |
| 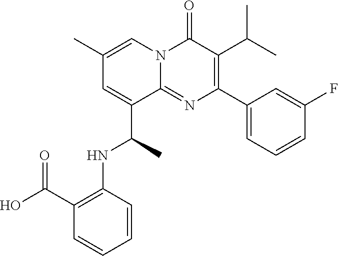 [SMILES: Cc1cc([C@@H](C)Nc2ccccc2C(=O)O)c2nc(-c3cccc(F)c3)c(C(C)C)c(=O)n2c1] | 1277 |
| 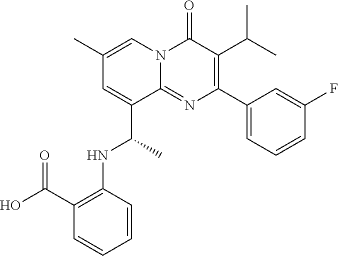 [SMILES: Cc1cc([C@H](C)Nc2ccccc2C(=O)O)c2nc(-c3cccc(F)c3)c(C(C)C)c(=O)n2c1] | 1278 |
| 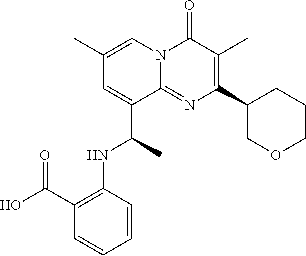 [SMILES: Cc1cc([C@@H](C)Nc2ccccc2C(=O)O)c2nc([C@H]3CCCOC3)c(C)c(=O)n2c1] | 1279 |
| 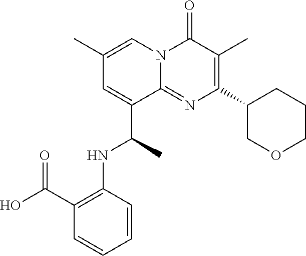 [SMILES: Cc1cc([C@@H](C)Nc2ccccc2C(=O)O)c2nc([C@@H]3CCCOC3)c(C)c(=O)n2c1] | 1280 |
| 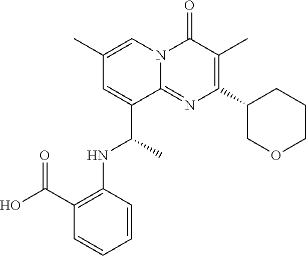 [SMILES: Cc1cc([C@H](C)Nc2ccccc2C(=O)O)c2nc([C@@H]3CCCOC3)c(C)c(=O)n2c1] | 1281 |
| 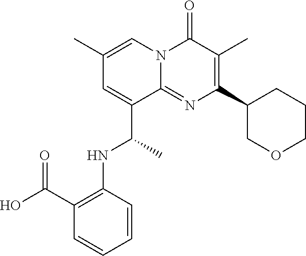 [SMILES: Cc1cc([C@H](C)Nc2ccccc2C(=O)O)c2nc([C@H]3CCCOC3)c(C)c(=O)n2c1] | 1282 |
| 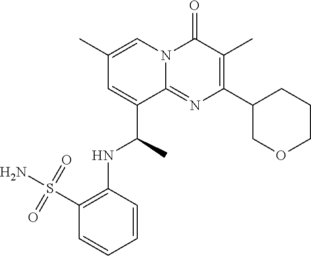 [SMILES: Cc1cc([C@@H](C)Nc2ccccc2S(N)(=O)=O)c2nc(C3CCCOC3)c(C)c(=O)n2c1] | 1283 |
| 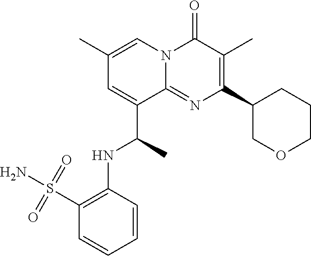 [SMILES: Cc1cc([C@@H](C)Nc2ccccc2S(N)(=O)=O)c2nc([C@H]3CCCOC3)c(C)c(=O)n2c1] | 1284 |
| 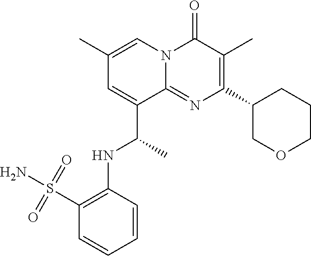 [SMILES: Cc1cc([C@H](C)Nc2ccccc2S(N)(=O)=O)c2nc([C@@H]3CCCOC3)c(C)c(=O)n2c1] | 1285 |
| 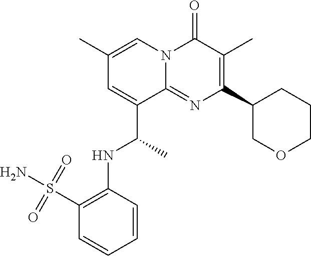 [SMILES: Cc1cc([C@H](C)Nc2ccccc2S(N)(=O)=O)c2nc([C@H]3CCCOC3)c(C)c(=O)n2c1] | 1286 |
| 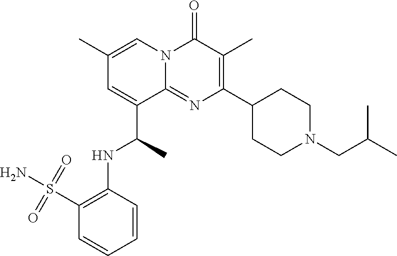 [SMILES: Cc1cc([C@@H](C)Nc2ccccc2S(N)(=O)=O)c2nc(C3CCN(CC(C)C)CC3)c(C)c(=O)n2c1] | 1287 |
| 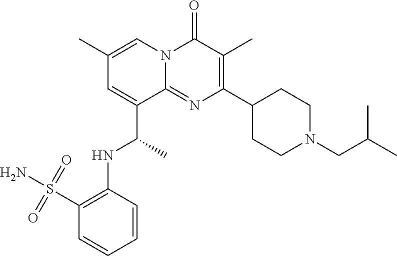 [SMILES: Cc1cc([C@H](C)Nc2ccccc2S(N)(=O)=O)c2nc(C3CCN(CC(C)C)CC3)c(C)c(=O)n2c1] | 1288 |
| 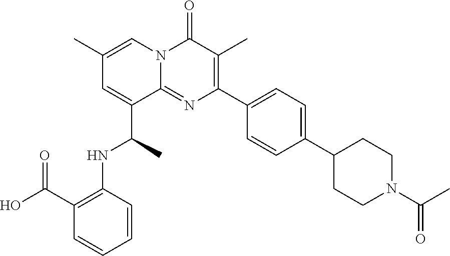 [SMILES: CC(=O)N1CCC(c2ccc(-c3nc4c([C@@H](C)Nc5ccccc5C(=O)O)cc(C)cn4c(=O)c3C)cc2)CC1] | 1289 |
| 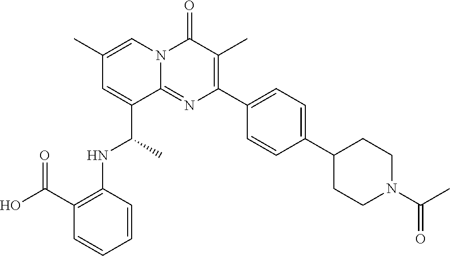 [SMILES: CC(=O)N1CCC(c2ccc(-c3nc4c([C@H](C)Nc5ccccc5C(=O)O)cc(C)cn4c(=O)c3C)cc2)CC1] | 1290 |
| 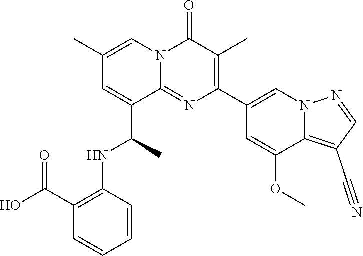 [SMILES: COc1cc(-c2nc3c([C@@H](C)Nc4ccccc4C(=O)O)cc(C)cn3c(=O)c2C)cn2ncc(C#N)c12] | 1291 |
| 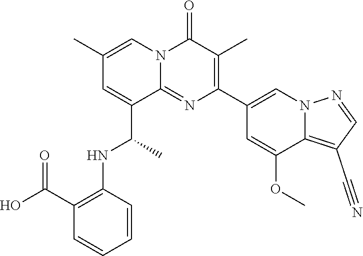 [SMILES: COc1cc(-c2nc3c([C@H](C)Nc4ccccc4C(=O)O)cc(C)cn3c(=O)c2C)cn2ncc(C#N)c12] | 1292 |
| 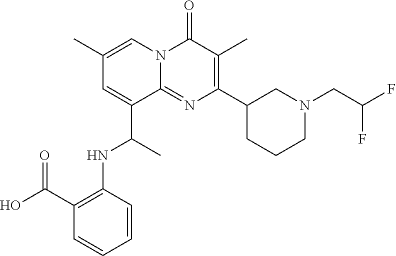 [SMILES: Cc1cc(C(C)Nc2ccccc2C(=O)O)c2nc(C3CCCN(CC(F)F)C3)c(C)c(=O)n2c1] | 1293 |
| 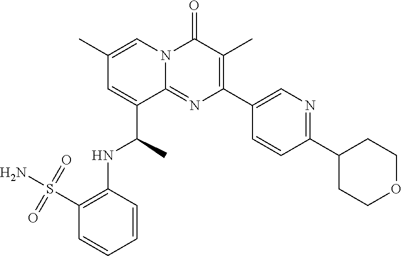 [SMILES: Cc1cc([C@@H](C)Nc2ccccc2S(N)(=O)=O)c2nc(-c3ccc(C4CCOCC4)nc3)c(C)c(=O)n2c1] | 1294 |
| 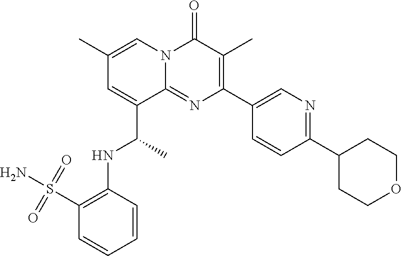 [SMILES: Cc1cc([C@H](C)Nc2ccccc2S(N)(=O)=O)c2nc(-c3ccc(C4CCOCC4)nc3)c(C)c(=O)n2c1] | 1295 |
| 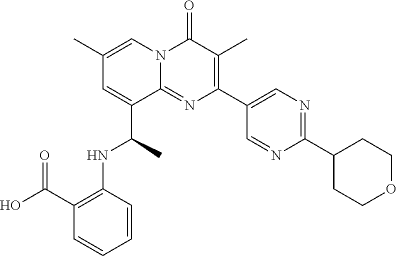 [SMILES: Cc1cc([C@@H](C)Nc2ccccc2C(=O)O)c2nc(-c3cnc(C4CCOCC4)nc3)c(C)c(=O)n2c1] | 1296 |
| 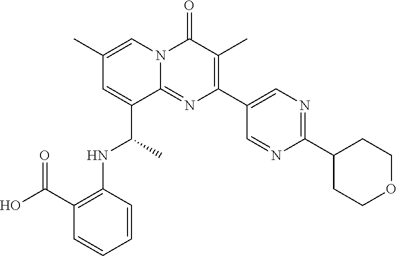 [SMILES: Cc1cc([C@H](C)Nc2ccccc2C(=O)O)c2nc(-c3cnc(C4CCOCC4)nc3)c(C)c(=O)n2c1] | 1297 |
| 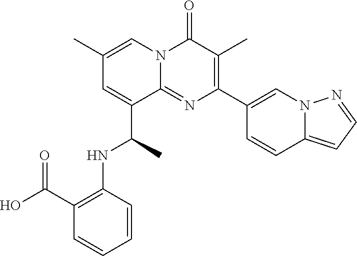 [SMILES: Cc1cc([C@@H](C)Nc2ccccc2C(=O)O)c2nc(-c3ccc4ccnn4c3)c(C)c(=O)n2c1] | 1298 |
| 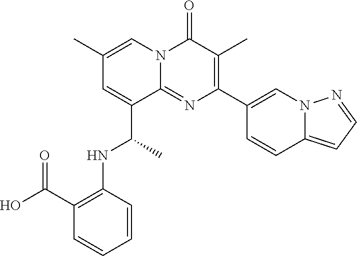 [SMILES: Cc1cc([C@H](C)Nc2ccccc2C(=O)O)c2nc(-c3ccc4ccnn4c3)c(C)c(=O)n2c1] | 1299 |
| 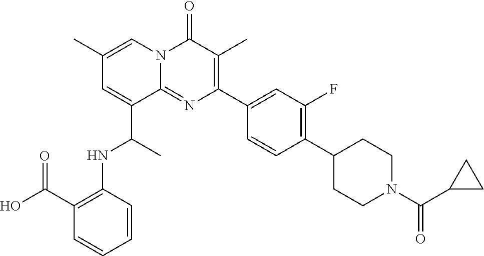 [SMILES: Cc1cc(C(C)Nc2ccccc2C(=O)O)c2nc(-c3ccc(C4CCN(C(=O)C5CC5)CC4)c(F)c3)c(C)c(=O)n2c1] | 1300 |
| 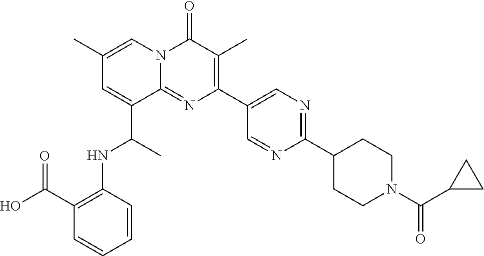 [SMILES: Cc1cc(C(C)Nc2ccccc2C(=O)O)c2nc(-c3cnc(C4CCN(C(=O)C5CC5)CC4)nc3)c(C)c(=O)n2c1] | 1301 |
| 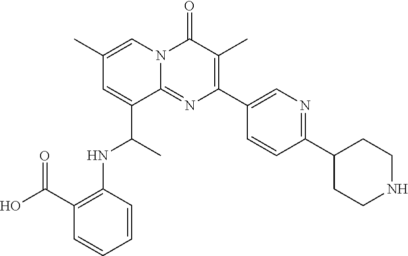 [SMILES: Cc1cc(C(C)Nc2ccccc2C(=O)O)c2nc(-c3ccc(C4CCNCC4)nc3)c(C)c(=O)n2c1] | 1302 |
| 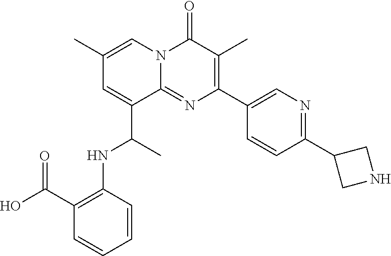 [SMILES: Cc1cc(C(C)Nc2ccccc2C(=O)O)c2nc(-c3ccc(C4CNC4)nc3)c(C)c(=O)n2c1] | 1303 |
| 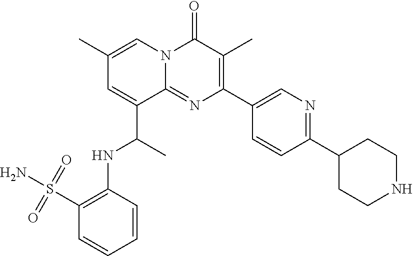 [SMILES: Cc1cc(C(C)Nc2ccccc2S(N)(=O)=O)c2nc(-c3ccc(C4CCNCC4)nc3)c(C)c(=O)n2c1] | 1304 |
| 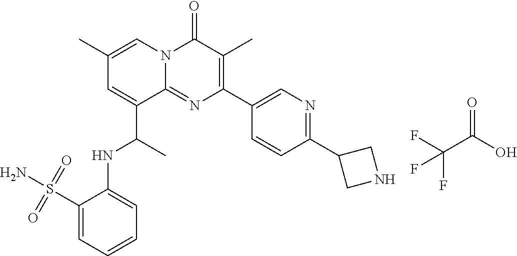 | 1305 |
| 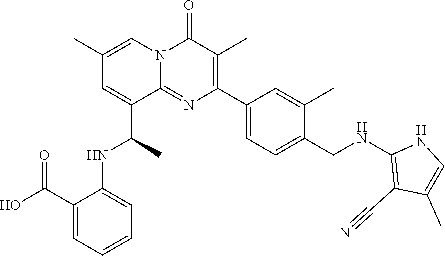 [SMILES: Cc1cc([C@@H](C)Nc2ccccc2C(=O)O)c2nc(-c3ccc(CNc4[nH]cc(C)c4C#N)c(C)c3)c(C)c(=O)n2c1] | 1306 |
| 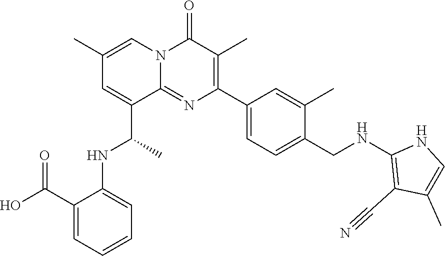 [SMILES: Cc1cc([C@H](C)Nc2ccccc2C(=O)O)c2nc(-c3ccc(CNc4[nH]cc(C)c4C#N)c(C)c3)c(C)c(=O)n2c1] | 1307 |
| 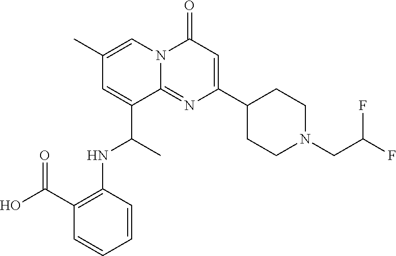 [SMILES: Cc1cc(C(C)Nc2ccccc2C(=O)O)c2nc(C3CCN(CC(F)F)CC3)cc(=O)n2c1] | 1308 |
| 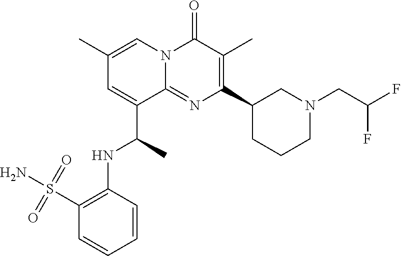 [SMILES: Cc1cc([C@@H](C)Nc2ccccc2S(N)(=O)=O)c2nc([C@@H]3CCCN(CC(F)F)C3)c(C)c(=O)n2c1] | 1309 |
| 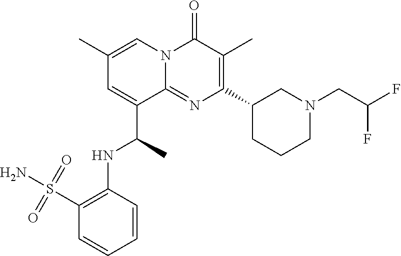 [SMILES: Cc1cc([C@@H](C)Nc2ccccc2S(N)(=O)=O)c2nc([C@H]3CCCN(CC(F)F)C3)c(C)c(=O)n2c1] | 1310 |
| 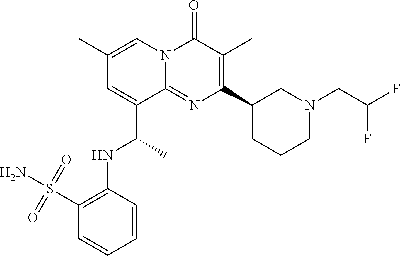 [SMILES: Cc1cc([C@H](C)Nc2ccccc2S(N)(=O)=O)c2nc([C@@H]3CCCN(CC(F)F)C3)c(C)c(=O)n2c1] | 1311 |
| 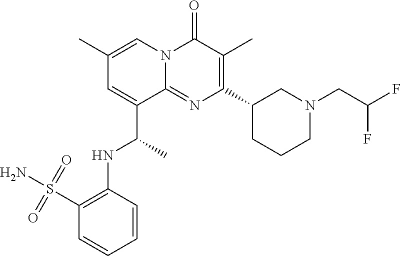 [SMILES: Cc1cc([C@H](C)Nc2ccccc2S(N)(=O)=O)c2nc([C@H]3CCCN(CC(F)F)C3)c(C)c(=O)n2c1] | 1312 |
| 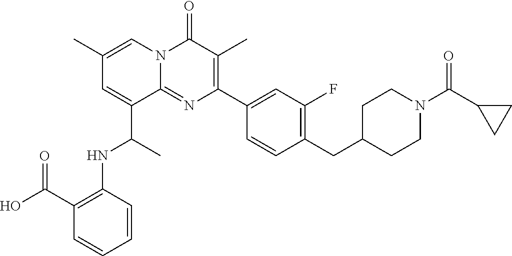 [SMILES: Cc1cc(C(C)Nc2ccccc2C(=O)O)c2nc(-c3ccc(CC4CCN(C(=O)C5CC5)CC4)c(F)c3)c(C)c(=O)n2c1] | 1313 |
| 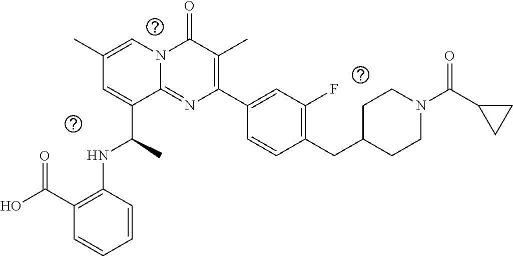 [SMILES: Cc1cc([C@@H](C)Nc2ccccc2C(=O)O)c2nc(-c3ccc(CC4CCN(C(=O)C5CC5)CC4)c(F)c3)c(C)c(=O)n2c1] | 1314 |
| 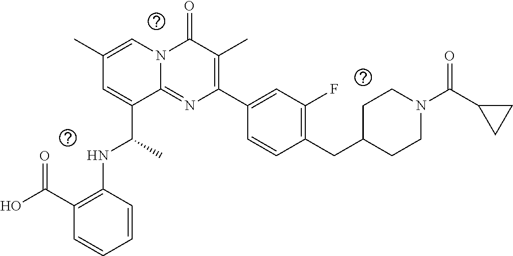 [SMILES: Cc1cc([C@H](C)Nc2ccccc2C(=O)O)c2nc(-c3ccc(CC4CCN(C(=O)C5CC5)CC4)c(F)c3)c(C)c(=O)n2c1] | 1315 |
| 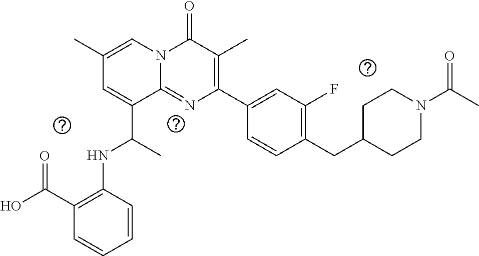 [SMILES: CC(=O)N1CCC(Cc2ccc(-c3nc4c(C(C)Nc5ccccc5C(=O)O)cc(C)cn4c(=O)c3C)cc2F)CC1] | 1316 |
| 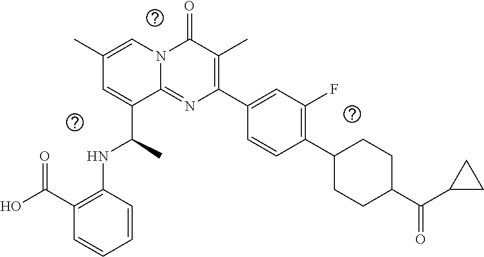 [SMILES: Cc1cc([C@@H](C)Nc2ccccc2C(=O)O)c2nc(-c3ccc(C4CCC(C(=O)C5CC5)CC4)c(F)c3)c(C)c(=O)n2c1] | 1317 |
| 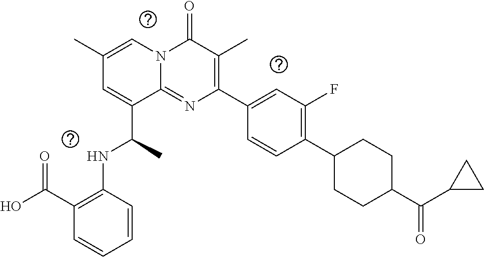 [SMILES: Cc1cc([C@@H](C)Nc2ccccc2C(=O)O)c2nc(-c3ccc(C4CCC(C(=O)C5CC5)CC4)c(F)c3)c(C)c(=O)n2c1] | 1318 |
| 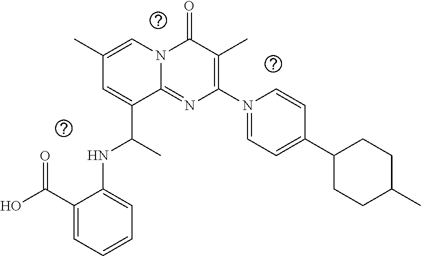 | 1319 |
| 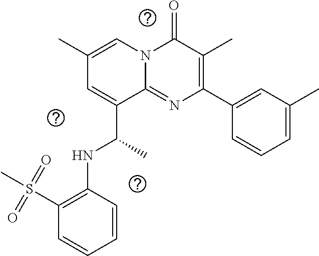 | 1320 |
| 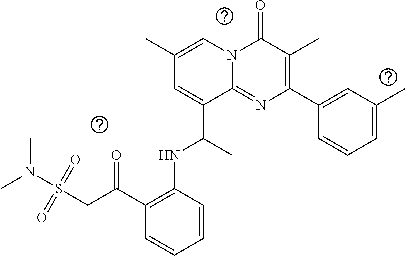 | 1321 |
| 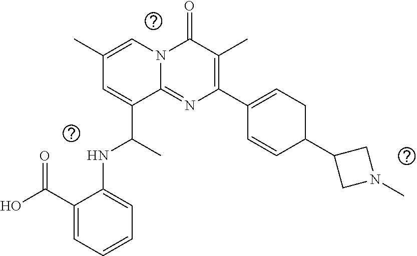 [SMILES: Cc1cc(C(C)Nc2ccccc2C(=O)O)c2nc(C3=CCC(C4CN(C)C4)C=C3)c(C)c(=O)n2c1] | 1322 |
| 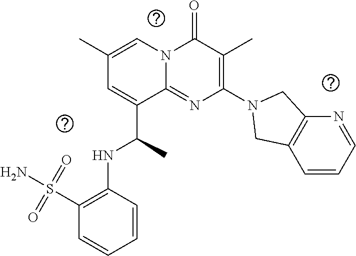 [SMILES: Cc1cc([C@@H](C)Nc2ccccc2S(N)(=O)=O)c2nc(N3Cc4cccnc4C3)c(C)c(=O)n2c1] | 1323 |
| 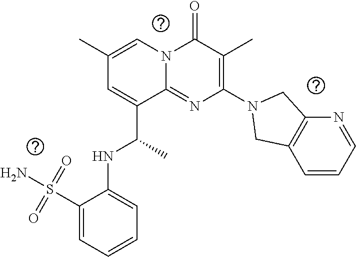 [SMILES: Cc1cc([C@H](C)Nc2ccccc2S(N)(=O)=O)c2nc(N3Cc4cccnc4C3)c(C)c(=O)n2c1] | 1324 |
| 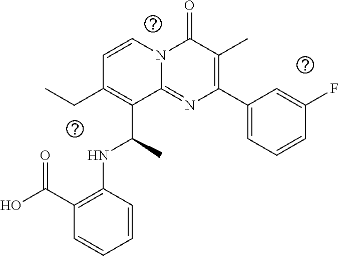 [SMILES: CCc1ccn2c(=O)c(C)c(-c3cccc(F)c3)nc2c1[C@@H](C)Nc1ccccc1C(=O)O] | 1325 |
| 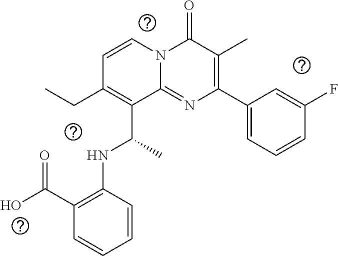 [SMILES: CCc1ccn2c(=O)c(C)c(-c3cccc(F)c3)nc2c1[C@H](C)Nc1ccccc1C(=O)O] | 1326 |
| 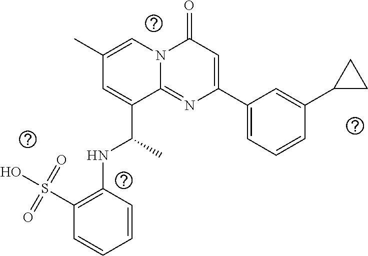 [SMILES: Cc1cc([C@H](C)Nc2ccccc2S(=O)(=O)O)c2nc(-c3cccc(C4CC4)c3)cc(=O)n2c1] | 1327 |
| 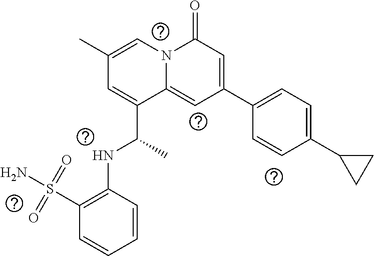 [SMILES: Cc1cc([C@H](C)Nc2ccccc2S(N)(=O)=O)c2cc(-c3ccc(C4CC4)cc3)cc(=O)n2c1] | 1328 |
| 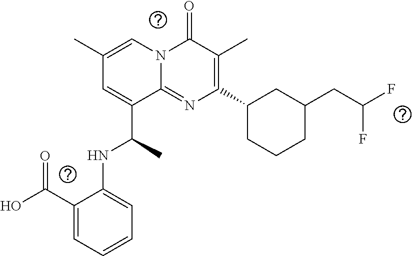 [SMILES: Cc1cc([C@@H](C)Nc2ccccc2C(=O)O)c2nc([C@H]3CCCC(CC(F)F)C3)c(C)c(=O)n2c1] | 1329 |
| 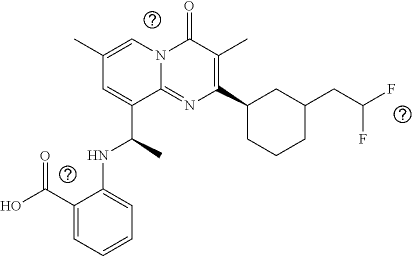 [SMILES: Cc1cc([C@@H](C)Nc2ccccc2C(=O)O)c2nc([C@@H]3CCCC(CC(F)F)C3)c(C)c(=O)n2c1] | 1330 |
| 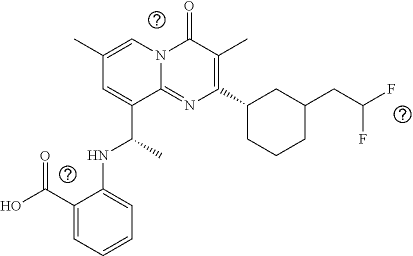 [SMILES: Cc1cc([C@H](C)Nc2ccccc2C(=O)O)c2nc([C@H]3CCCC(CC(F)F)C3)c(C)c(=O)n2c1] | 1331 |
| 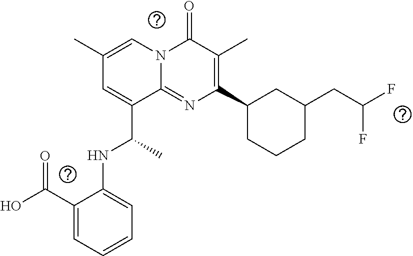 [SMILES: Cc1cc([C@H](C)Nc2ccccc2C(=O)O)c2nc([C@@H]3CCCC(CC(F)F)C3)c(C)c(=O)n2c1] | 1332 |
| 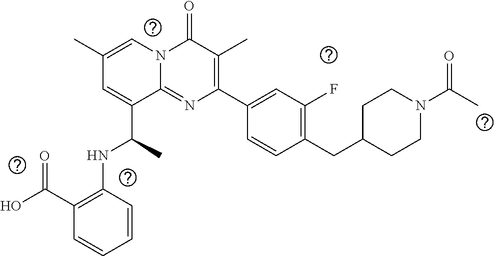 [SMILES: CC(=O)N1CCC(Cc2ccc(-c3nc4c([C@@H](C)Nc5ccccc5C(=O)O)cc(C)cn4c(=O)c3C)cc2F)CC1] | 1333 |
| 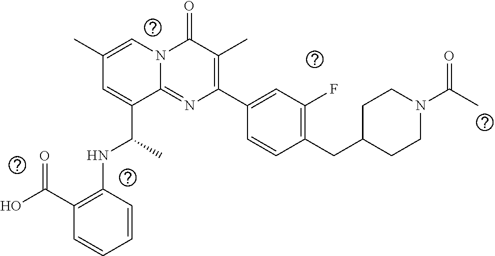 [SMILES: CC(=O)N1CCC(Cc2ccc(-c3nc4c([C@H](C)Nc5ccccc5C(=O)O)cc(C)cn4c(=O)c3C)cc2F)CC1] | 1334 |
| 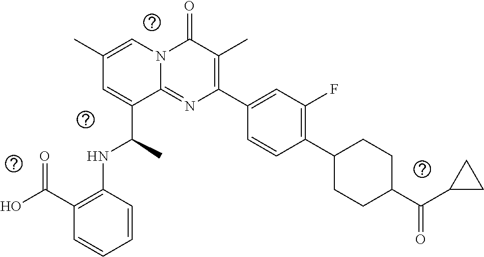 [SMILES: Cc1cc([C@@H](C)Nc2ccccc2C(=O)O)c2nc(-c3ccc(C4CCC(C(=O)C5CC5)CC4)c(F)c3)c(C)c(=O)n2c1] | 1335 |
| 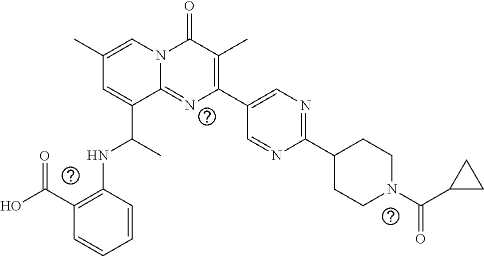 [SMILES: Cc1cc(C(C)Nc2ccccc2C(=O)O)c2nc(-c3cnc(C4CCN(C(=O)C5CC5)CC4)nc3)c(C)c(=O)n2c1] | 1336 |
| 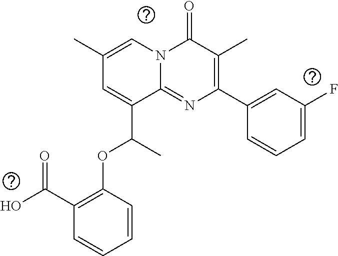 [SMILES: Cc1cc(C(C)Oc2ccccc2C(=O)O)c2nc(-c3cccc(F)c3)c(C)c(=O)n2c1] | 1337 |
| 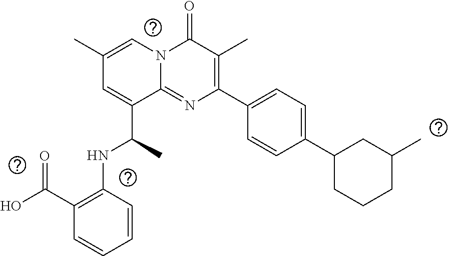 [SMILES: Cc1cc([C@@H](C)Nc2ccccc2C(=O)O)c2nc(-c3ccc(C4CCCC(C)C4)cc3)c(C)c(=O)n2c1] | 1338 |
| 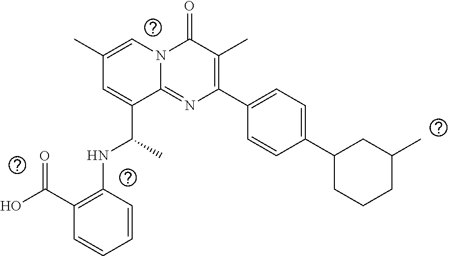 [SMILES: Cc1cc([C@H](C)Nc2ccccc2C(=O)O)c2nc(-c3ccc(C4CCCC(C)C4)cc3)c(C)c(=O)n2c1] | 1339 |
| 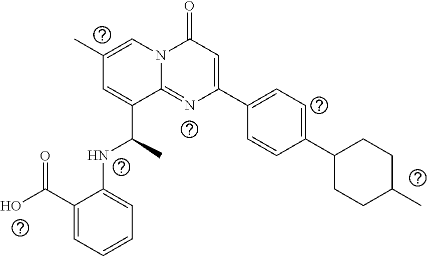 [SMILES: Cc1cc([C@@H](C)Nc2ccccc2C(=O)O)c2nc(-c3ccc(C4CCC(C)CC4)cc3)cc(=O)n2c1] | 1340 |
| 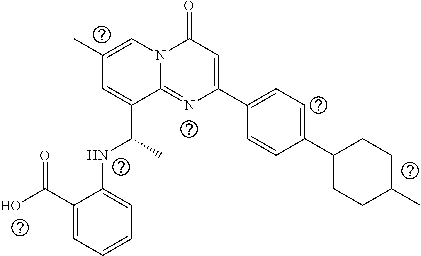 [SMILES: Cc1cc([C@H](C)Nc2ccccc2C(=O)O)c2nc(-c3ccc(C4CCC(C)CC4)cc3)cc(=O)n2c1] | 1341 |
| 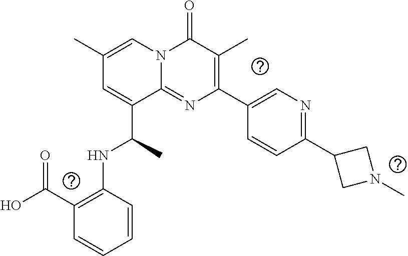 [SMILES: Cc1cc([C@@H](C)Nc2ccccc2C(=O)O)c2nc(-c3ccc(C4CN(C)C4)nc3)c(C)c(=O)n2c1] | 1342 |
| 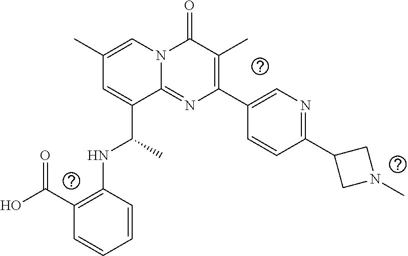 [SMILES: Cc1cc([C@H](C)Nc2ccccc2C(=O)O)c2nc(-c3ccc(C4CN(C)C4)nc3)c(C)c(=O)n2c1] | 1343 |
| 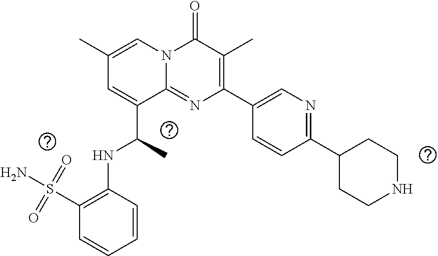 [SMILES: Cc1cc([C@@H](C)Nc2ccccc2S(N)(=O)=O)c2nc(-c3ccc(C4CCNCC4)nc3)c(C)c(=O)n2c1] | 1344 |
| 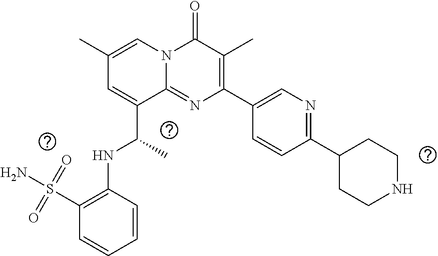 [SMILES: Cc1cc([C@H](C)Nc2ccccc2S(N)(=O)=O)c2nc(-c3ccc(C4CCNCC4)nc3)c(C)c(=O)n2c1] | 1345 |
| 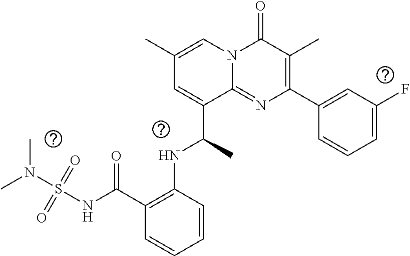 [SMILES: Cc1cc([C@@H](C)Nc2ccccc2C(=O)NS(=O)(=O)N(C)C)c2nc(-c3cccc(F)c3)c(C)c(=O)n2c1] | 1346 |
| 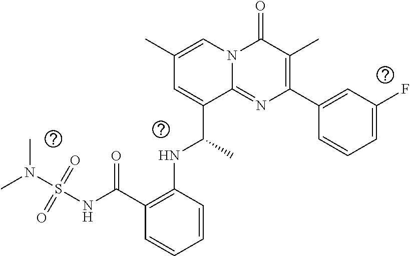 [SMILES: Cc1cc([C@H](C)Nc2ccccc2C(=O)NS(=O)(=O)N(C)C)c2nc(-c3cccc(F)c3)c(C)c(=O)n2c1] | 1347 |
| 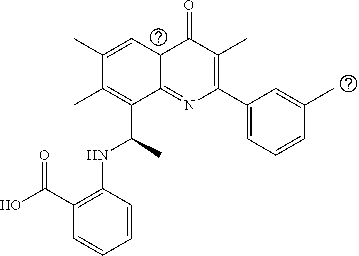 [SMILES: CC1=CC2C(=O)C(C)=C(c3cccc(C)c3)N=C2C([C@@H](C)Nc2ccccc2C(=O)O)=C1C] | 1348 |
| 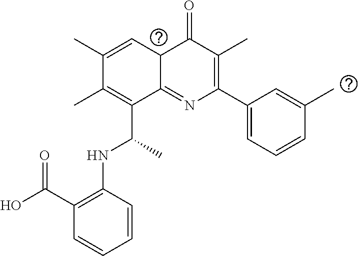 [SMILES: CC1=CC2C(=O)C(C)=C(c3cccc(C)c3)N=C2C([C@H](C)Nc2ccccc2C(=O)O)=C1C] | 1349 |
| 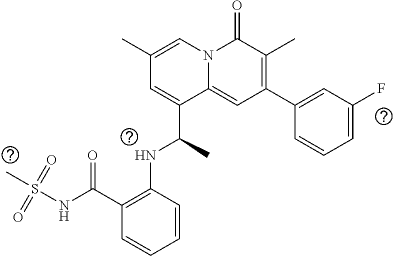 [SMILES: Cc1cc([C@@H](C)Nc2ccccc2C(=O)NS(C)(=O)=O)c2cc(-c3cccc(F)c3)c(C)c(=O)n2c1] | 1350 |
| 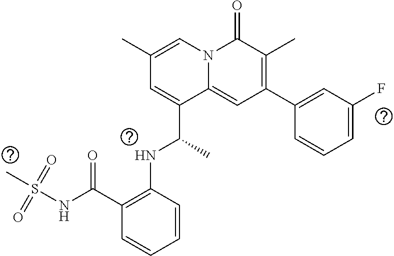 [SMILES: Cc1cc([C@H](C)Nc2ccccc2C(=O)NS(C)(=O)=O)c2cc(-c3cccc(F)c3)c(C)c(=O)n2c1] | 1351 |
| 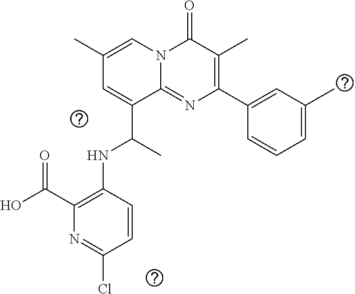 [SMILES: Cc1cccc(-c2nc3c(C(C)Nc4ccc(Cl)nc4C(=O)O)cc(C)cn3c(=O)c2C)c1] | 1352 |
| 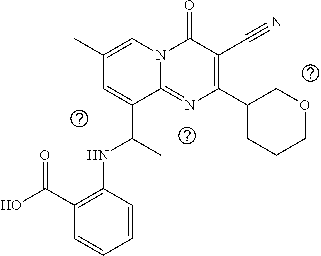 [SMILES: Cc1cc(C(C)Nc2ccccc2C(=O)O)c2nc(C3CCCOC3)c(C#N)c(=O)n2c1] | 1353 |
| 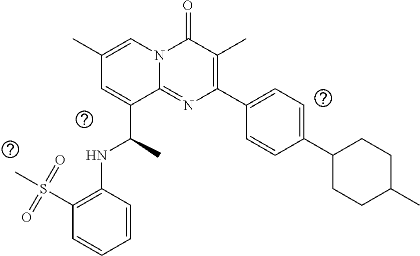 | 1354 |
| 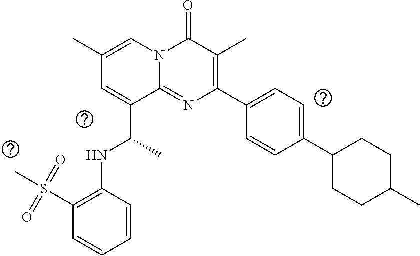 | 1355 |
| 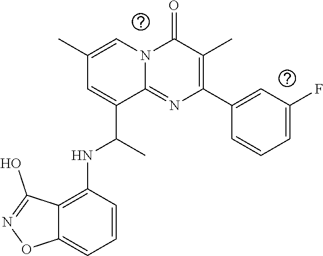 [SMILES: Cc1cc(C(C)Nc2cccc3onc(O)c23)c2nc(-c3cccc(F)c3)c(C)c(=O)n2c1] | 1356 |
| 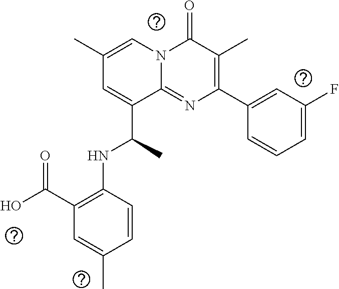 [SMILES: Cc1ccc(N[C@H](C)c2cc(C)cn3c(=O)c(C)c(-c4cccc(F)c4)nc23)c(C(=O)O)c1] | 1357 |
| 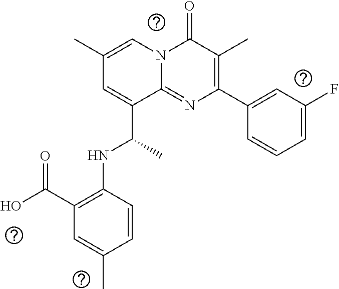 [SMILES: Cc1ccc(N[C@@H](C)c2cc(C)cn3c(=O)c(C)c(-c4cccc(F)c4)nc23)c(C(=O)O)c1] | 1358 |
| 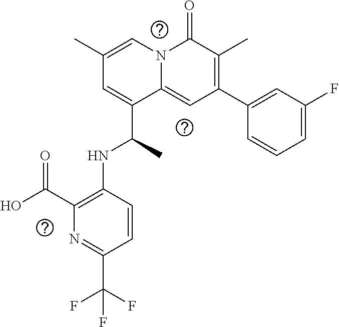 [SMILES: Cc1cc([C@@H](C)Nc2ccc(C(F)(F)F)nc2C(=O)O)c2cc(-c3cccc(F)c3)c(C)c(=O)n2c1] | 1359 |
| 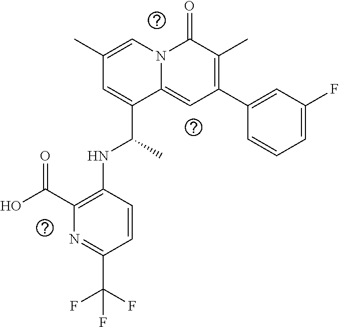 [SMILES: Cc1cc([C@H](C)Nc2ccc(C(F)(F)F)nc2C(=O)O)c2cc(-c3cccc(F)c3)c(C)c(=O)n2c1] | 1360 |
| 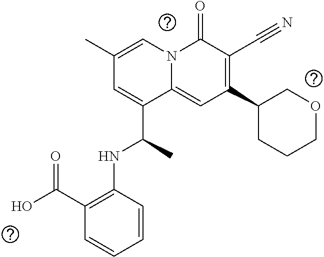 [SMILES: Cc1cc([C@@H](C)Nc2ccccc2C(=O)O)c2cc([C@@H]3CCCOC3)c(C#N)c(=O)n2c1] | 1361 |
| 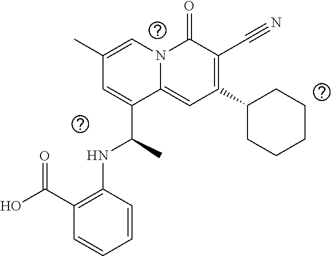 | 1362 |
| 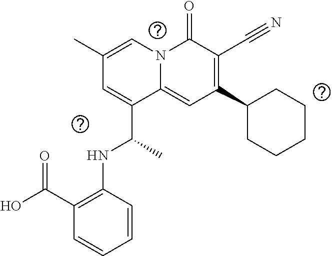 | 1363 |
| 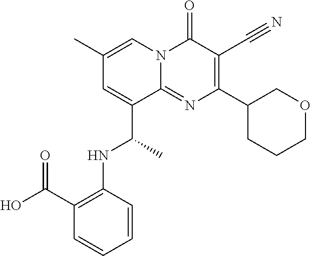 [SMILES: Cc1cc([C@H](C)Nc2ccccc2C(=O)O)c2nc(C3CCCOC3)c(C#N)c(=O)n2c1] | 1364 |
| 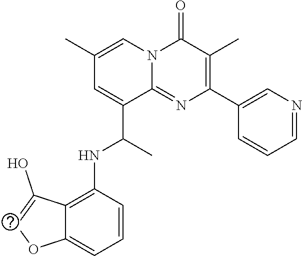 [SMILES: Cc1cc(C(C)Nc2cccc3occ(O)c23)c2nc(-c3cccnc3)c(C)c(=O)n2c1] | 1365 |
| 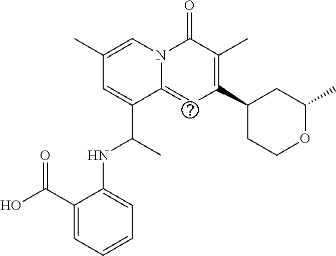 [SMILES: Cc1cc(C(C)Nc2ccccc2C(=O)O)c2cc([C@@H]3CCO[C@@H](C)C3)c(C)c(=O)n2c1] | 1366 |
| 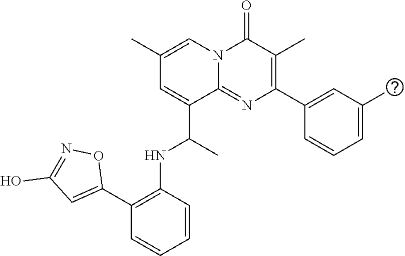 [SMILES: Cc1cccc(-c2nc3c(C(C)Nc4ccccc4-c4cc(O)no4)cc(C)cn3c(=O)c2C)c1] | 1367 |
| 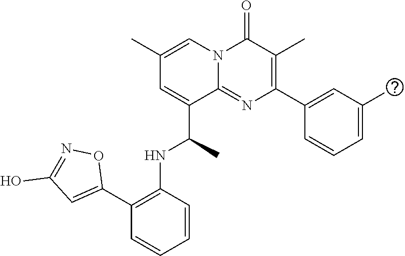 [SMILES: Cc1cccc(-c2nc3c([C@@H](C)Nc4ccccc4-c4cc(O)no4)cc(C)cn3c(=O)c2C)c1] | 1368 |
| 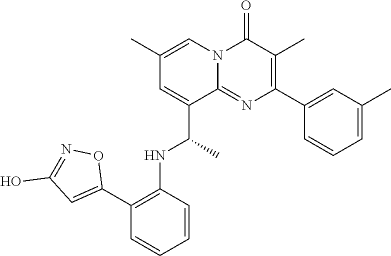 [SMILES: Cc1cccc(-c2nc3c([C@H](C)Nc4ccccc4-c4cc(O)no4)cc(C)cn3c(=O)c2C)c1] | 1369 |
| 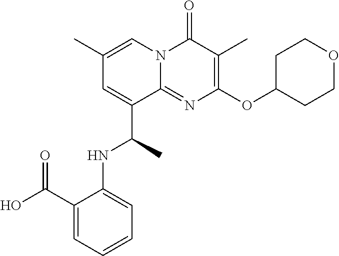 [SMILES: Cc1cc([C@@H](C)Nc2ccccc2C(=O)O)c2nc(OC3CCOCC3)c(C)c(=O)n2c1] | 1370 |
| 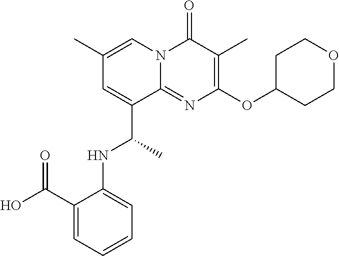 [SMILES: Cc1cc([C@H](C)Nc2ccccc2C(=O)O)c2nc(OC3CCOCC3)c(C)c(=O)n2c1] | 1371 |
| 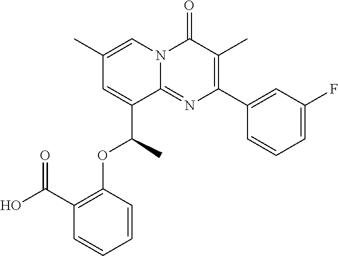 [SMILES: Cc1cc([C@@H](C)Oc2ccccc2C(=O)O)c2nc(-c3cccc(F)c3)c(C)c(=O)n2c1] | 1372 |
| 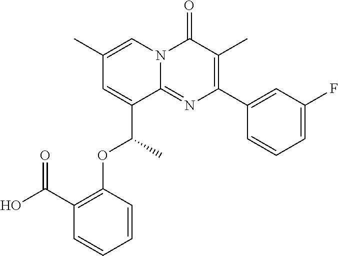 [SMILES: Cc1cc([C@H](C)Oc2ccccc2C(=O)O)c2nc(-c3cccc(F)c3)c(C)c(=O)n2c1] | 1373 |
| 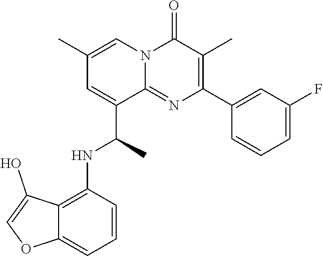 [SMILES: Cc1cc([C@@H](C)Nc2cccc3occ(O)c23)c2nc(-c3cccc(F)c3)c(C)c(=O)n2c1] | 1374 |
| 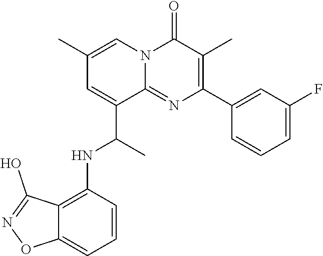 [SMILES: Cc1cc(C(C)Nc2cccc3onc(O)c23)c2nc(-c3cccc(F)c3)c(C)c(=O)n2c1] | 1375 |
| 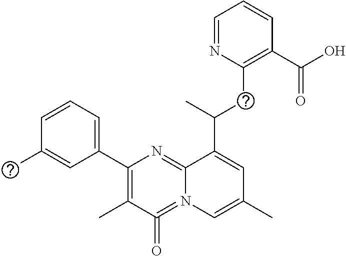 | 1376 |
| 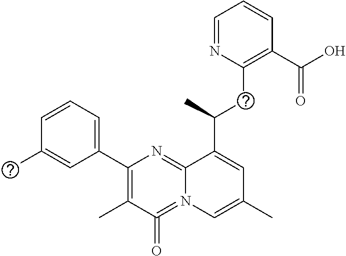 | 1377 |
| 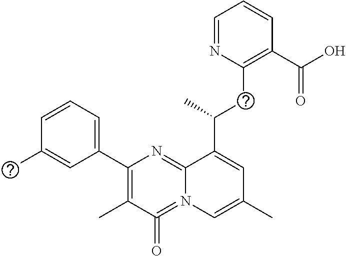 | 1378 |
| 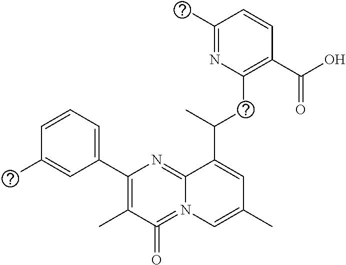 | 1379 |
| 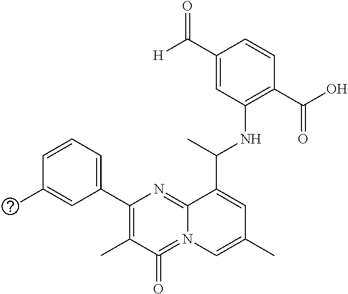 | 1380 |
| 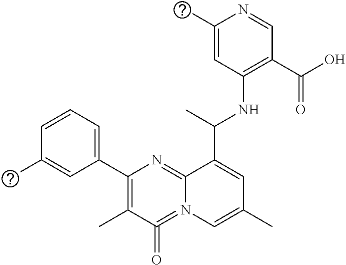 | 1381 |
| 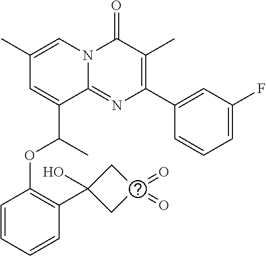 | 1382 |
| 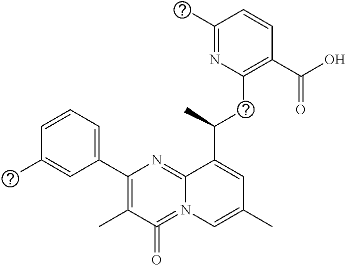 | 1383 |
| 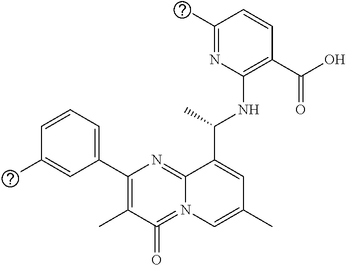 | 1384 |
| 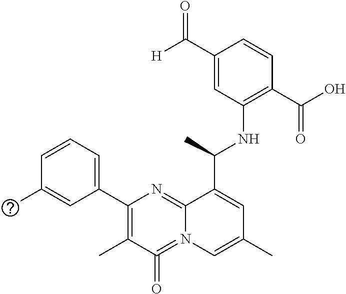 | 1385 |
| 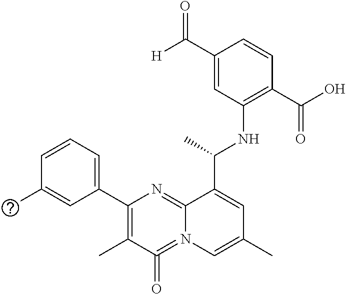 | 1386 |
| 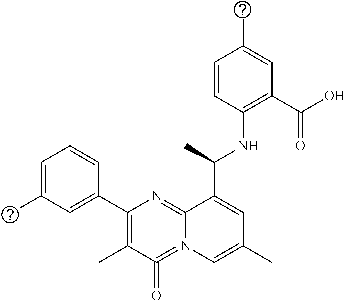 | 1387 |
| 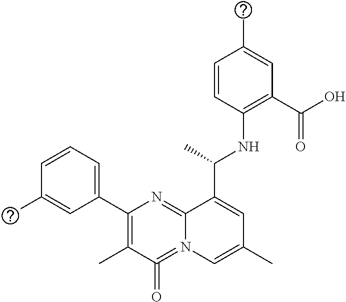 | 1388 |
| 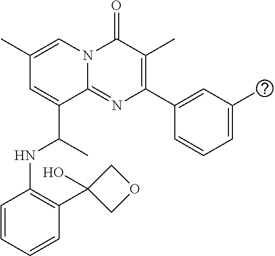 [SMILES: Cc1cccc(-c2nc3c(C(C)Nc4ccccc4C4(O)COC4)cc(C)cn3c(=O)c2C)c1] | 1389 |
| 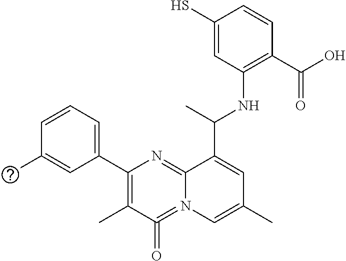 | 1390 |
| 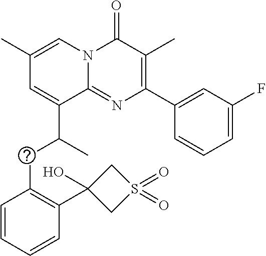 [SMILES: Cc1cc(C(C)Cc2ccccc2C2(O)CS(=O)(=O)C2)c2nc(-c3cccc(F)c3)c(C)c(=O)n2c1] | 1391 |
| 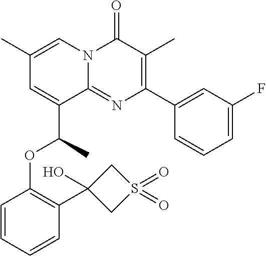 [SMILES: Cc1cc([C@@H](C)Oc2ccccc2C2(O)CS(=O)(=O)C2)c2nc(-c3cccc(F)c3)c(C)c(=O)n2c1] | 1392 |
| 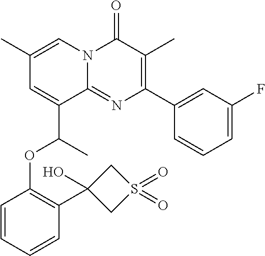 [SMILES: Cc1cc(C(C)Oc2ccccc2C2(O)CS(=O)(=O)C2)c2nc(-c3cccc(F)c3)c(C)c(=O)n2c1] | 1393 |
| 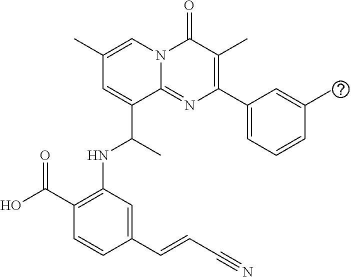 [SMILES: Cc1cccc(-c2nc3c(C(C)Nc4cc(/C=C/C#N)ccc4C(=O)O)cc(C)cn3c(=O)c2C)c1] | 1394 |
| 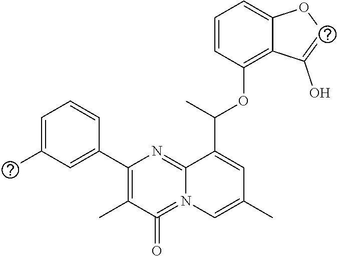 | 1395 |
| 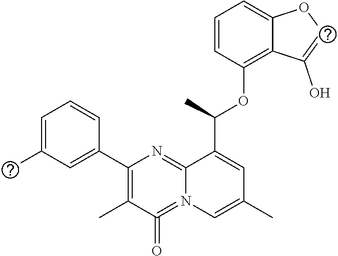 | 1396 |
| 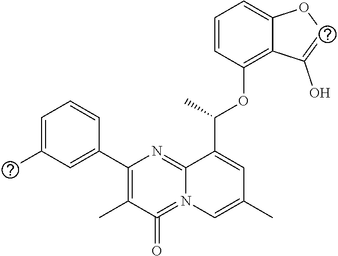 | 1397 |
| 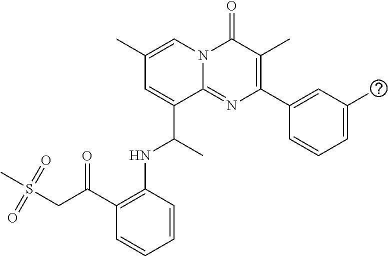 | 1398 |
| 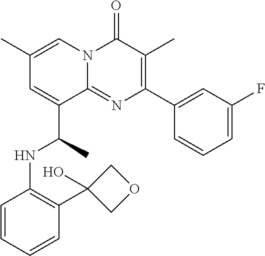 [SMILES: Cc1cc([C@@H](C)Nc2ccccc2C2(O)COC2)c2nc(-c3cccc(F)c3)c(C)c(=O)n2c1] | 1399 |
| 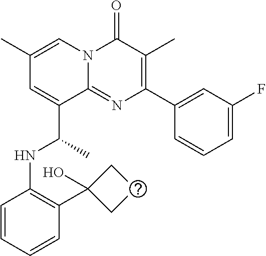 [SMILES: Cc1cc([C@H](C)Nc2ccccc2C2(O)CCC2)c2nc(-c3cccc(F)c3)c(C)c(=O)n2c1] | 1400 |
| 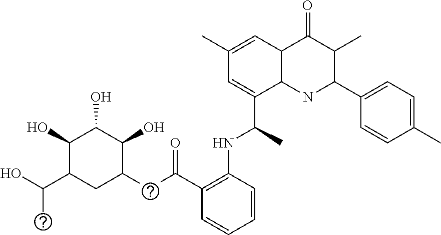 [SMILES: CC1=CC2C(=O)C(C)C(c3ccc(C)cc3)NC2C([C@@H](C)Nc2ccccc2C(=O)CC2CC(C(C)O)[C@@H](O)[C@H](O)[C@H]2O)=C1] | 1401 |
| 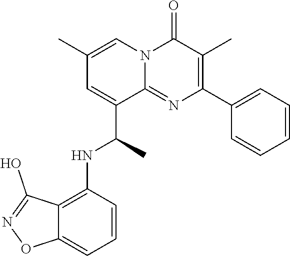 [SMILES: Cc1cc([C@@H](C)Nc2cccc3onc(O)c23)c2nc(-c3ccccc3)c(C)c(=O)n2c1] | 1402 |
| 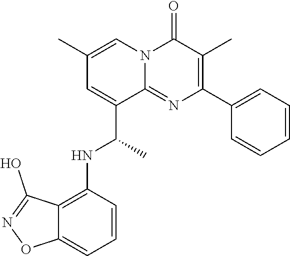 [SMILES: Cc1cc([C@H](C)Nc2cccc3onc(O)c23)c2nc(-c3ccccc3)c(C)c(=O)n2c1] | 1403 |
| 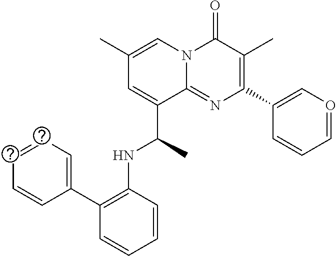 | 1404 |
| 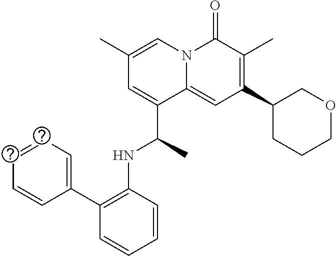 [SMILES: Cc1cc([C@@H](C)Nc2ccccc2-c2ccccc2)c2cc([C@@H]3CCCOC3)c(C)c(=O)n2c1] | 1405 |
| 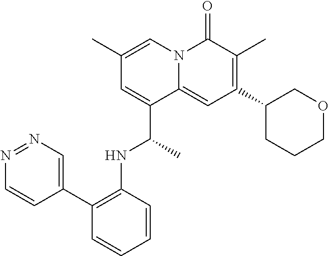 [SMILES: Cc1cc([C@H](C)Nc2ccccc2-c2ccnnc2)c2cc([C@H]3CCCOC3)c(C)c(=O)n2c1] | 1406 |
| 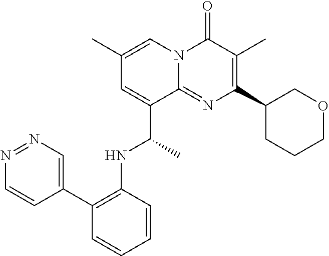 [SMILES: Cc1cc([C@H](C)Nc2ccccc2-c2ccnnc2)c2nc([C@@H]3CCCOC3)c(C)c(=O)n2c1] | 1407 |
| 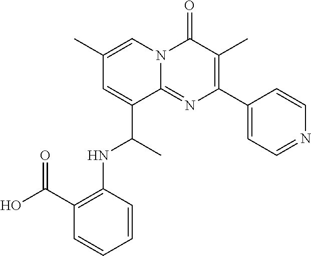 [SMILES: Cc1cc(C(C)Nc2ccccc2C(=O)O)c2nc(-c3ccncc3)c(C)c(=O)n2c1] | 1408 |
| 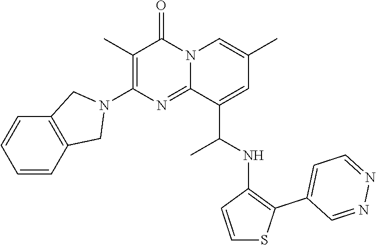 [SMILES: Cc1cc(C(C)Nc2ccsc2-c2ccnnc2)c2nc(N3Cc4ccccc4C3)c(C)c(=O)n2c1] | 1409 |
| 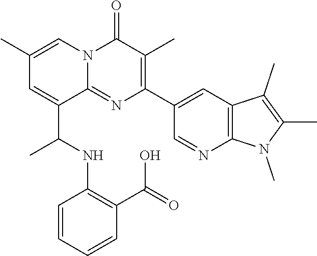 | 1410 |
| 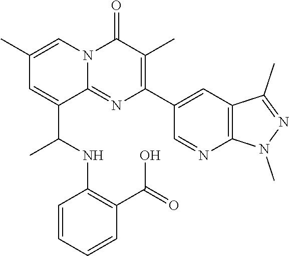 [SMILES: Cc1cc(C(C)Nc2ccccc2C(=O)O)c2nc(-c3cnc4c(c3)c(C)nn4C)c(C)c(=O)n2c1] | 1411 |
| 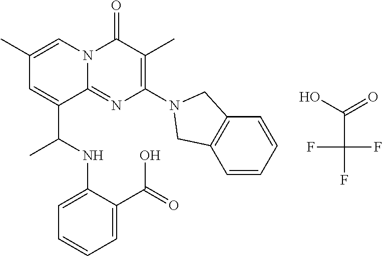 | 1412 |
| 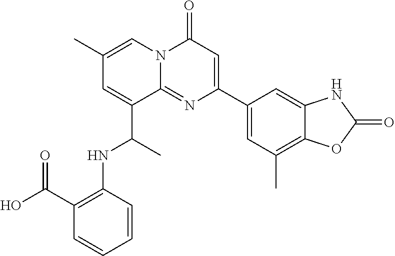 [SMILES: Cc1cc(C(C)Nc2ccccc2C(=O)O)c2nc(-c3cc(C)c4oc(=O)[nH]c4c3)cc(=O)n2c1] | 1413 |

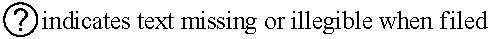

### Pharmaceutical Compositions

Some embodiments provide a pharmaceutical composition comprising a compound of Formula (I), or a pharmaceutically acceptable salt thereof, and one or more pharmaceutically acceptable excipients.

### Methods of Treatment

A “PI3Kα inhibitor” as used herein (e.g., compounds of Formula (I) and pharmaceutically acceptable salts thereof) includes any compound exhibiting PI3Kα inactivation activity (e.g., inhibiting or decreasing). In some embodiments, a PI3Kα inhibitor can be selective for a PI3Kα having one or more mutations.

The ability of test compounds to act as inhibitors of PI3Kα may be demonstrated by assays known in the art. The activity of the compounds and compositions provided herein as PI3Kα inhibitors can be assayed in vitro, in vivo, or in a cell line. In vitro assays include assays that determine inhibition of the kinase. Alternate in vitro assays quantitate the ability of the inhibitor to bind to the protein kinase and can be measured either by radio labeling the compound prior to binding, isolating the compound/kinase complex and determining the amount of radio label bound, or by running a competition experiment where new compounds are incubated with the kinase bound to known radio ligands.

Potency of a PI3Kα inhibitor as provided herein can be determined by EC₅₀ value. A compound with a lower EC₅₀ value, as determined under substantially similar conditions, is a more potent inhibitor relative to a compound with a higher EC₅₀ value.

Potency of a PI3Kα inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a PI3Kα-dependent phosphorylation level, in vitro or in vivo.

The selectivity between wild type PI3Kα and PI3Kα containing one or more mutations as described herein can also be measured using in vitro assays such as surface plasmon resonance and fluorence-based binding assays, and cellular assays such as the levels of pAKT, a biomarker of PI3Kα activity, and/or proliferation assays where cell proliferation is dependent on mutant PI3Kα kinase activity.

In some embodiments, the compounds provided herein can exhibit potent and selective inhibition of PI3Kα. For example, the compounds provided herein can bind to the helical phosphatidylinositol kinase homology domain catalytic domain of PI3Kα. In some embodiments, the compounds provided herein can exhibit nanomolar potency against a PI3Kα kinase including one or more mutations, for example, the mutations in Table 1.

In some embodiments, the compounds of Formula (I), or a pharmaceutically acceptable salt thereof, can selectively target PI3Kα. For example, a compound of Formula (I), or a pharmaceutically acceptable salt thereof, can selectively target PI3Kα over another kinase or non-kinase target.

In some embodiments, a compound of Formula (I), or a pharmaceutically acceptable salt thereof, can exhibit greater inhibition of PI3Kα containing one or more mutations as described herein (e.g., one or more mutations as described in Table 1) relative to inhibition of wild type PI3Kα. In some embodiments, a compound of Formula (I), or a pharmaceutically acceptable salt thereof can exhibit at least 2-fold, 3-fold, 5-fold, 10-fold, 25-fold, 50-fold or 100-fold greater inhibition of PI3Kα containing one or more mutations as described herein relative to inhibition of wild type PI3Kα. In some embodiments, a compound of Formula (I), or a pharmaceutically acceptable salt thereof, can exhibit up to 1,000-fold greater inhibition of PI3Kα containing one or more mutations as described herein relative to inhibition of wild type PI3Kα. In some embodiments, a compound of Formula (I), or a pharmaceutically acceptable salt thereof, can exhibit up to 10,000-fold greater inhibition of PI3Kα having a combination of mutations described herein relative to inhibition of wild type PI3Kα.

In some embodiments, a compound of Formula (I), or a pharmaceutically acceptable salt thereof, can exhibit from about 2-fold to about 10-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 10-fold to about 100-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 100-fold to about 1,000-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 1000-fold to about 10,000-fold greater inhibition of PI3Kα containing one or more mutations as described herein relative to inhibition of wild type PI3Kα.

Compounds of Formula (I), or pharmaceutically acceptable salts thereof, are useful for treating diseases which can be treated with a PI3Kα inhibitor, such as PI3Kα-associated diseases, e.g., proliferative disorders such as cancers, including hematological cancers and solid tumors (e.g., advanced or metastatic solid tumors).

In some embodiments, the subject has been identified or diagnosed as having a cancer with a dysregulation of a PIK3CA gene, a PI3Kα protein, or expression or activity, or level of any of the same (a PI3Kα-associated cancer), for example, as determined using a regulatory agency-approved, e.g., FDA-approved, assay or kit. In some embodiments, the subject has a tumor that is positive for a dysregulation of a PIK3CA gene, a PI3Kα protein, or expression or activity, or level of any of the same (e.g., as determined using a regulatory agency-approved assay or kit). For example, the subject has a tumor that is positive for a mutation as described in Table 1. The subject can be a subject with a tumor(s) that is positive for a dysregulation of a PIK3CA gene, a PI3Kα protein, or expression or activity, or level of any of the same (e.g., identified as positive using a regulatory agency-approved, e.g., FDA-approved, assay or kit). The subject can be a subject whose tumors have a dysregulation of a PIK3CA gene, a PI3Kα protein, or expression or activity, or a level of the same (e.g., where the tumor is identified as such using a regulatory agency-approved, e.g., FDA-approved, kit or assay). In some embodiments, the subject is suspected of having a PI3Kα-associated cancer. In some embodiments, the subject has a clinical record indicating that the subject has a tumor that has a dysregulation of a PIK3CA gene, a PI3Kα protein, or expression or activity, or level of any of the same (and optionally the clinical record indicates that the subject should be treated with any of the compositions provided herein).

In certain embodiments, compounds of Formula (I), or pharmaceutically acceptable salts thereof, are useful for preventing diseases as defined herein such as cancer. The term “preventing” as used herein means to delay the onset, recurrence or spread, in whole or in part, of the disease as described herein, or a symptom thereof.

The term “PI3Kα-associated disease” as used herein refers to diseases associated with or having a dysregulation of a PIK3CA gene, a PI3Kα protein, or the expression or activity or level of any (e.g., one or more) of the same (e.g., any of the types of dysregulation of a PIK3CA gene, or a PI3Kα protein, or the expression or activity or level of any of the same described herein). Non-limiting examples of a PI3Kα-associated disease include, for example, proliferative disorders such as cancer (e.g., PI3Kα-associated cancer).

The term “PI3Kα-associated cancer” as used herein refers to cancers associated with or having a dysregulation of a PIK3CA gene, a PI3Kα protein, or expression or activity, or level of any of the same. Non-limiting examples of PI3Kα-associated cancer are described herein.

The phrase “dysregulation of a PIK3CA gene, a PI3Kα protein, or the expression or activity or level of any of the same” refers to a genetic mutation (e.g., a mutation in a PIK3CA gene that results in the expression of a PI3Kα that includes a deletion of at least one amino acid as compared to a wild type PI3Kα, a mutation in a PIK3CA gene that results in the expression of PI3Kα with one or more point mutations as compared to a wild type PI3Kα, a mutation in a PIK3CA gene that results in the expression of PI3Kα with at least one inserted amino acid as compared to a wild type PI3Kα, a gene duplication that results in an increased level of PI3Kα in a cell, or a mutation in a regulatory sequence (e.g., a promoter and/or enhancer) that results in an increased level of PI3Kα in a cell), an alternative spliced version of PI3Kα mRNA that results in PI3Kα having a deletion of at least one amino acid in the PI3Kα as compared to the wild type PI3Kα), or increased expression (e.g., increased levels) of a wild type PI3Kα in a mammalian cell due to aberrant cell signaling and/or dysregulated autocrine/paracrine signaling (e.g., as compared to a control non-cancerous cell). As another example, a dysregulation of a PIK3CA gene, a PI3Kα protein, or expression or activity, or level of any of the same, can be a mutation in a PIK3CA gene that encodes a PI3Kα that is constitutively active or has increased activity as compared to a protein encoded by a PIK3CA gene that does not include the mutation. Non-limiting examples of PI3Kα point mutations/substitutions/insertions/deletions are described in Table 1.

The term “wild type” describes a nucleic acid (e.g., a PIK3CA gene or a PI3Kα mRNA) or protein (e.g., a PI3Kα) sequence that is typically found in a subject that does not have a disease related to the reference nucleic acid or protein.

The term “wild type PI3Kα” or “wild-type PI3Kα” describes a normal PI3Kα nucleic acid (e.g., a PIK3CA or PI3Kα mRNA) or protein that is found in a subject that does not have a PI3Kα-associated disease, e.g., a PI3Kα-associated cancer (and optionally also does not have an increased risk of developing a PI3Kα-associated disease and/or is not suspected of having a PI3Kα-associated disease), or is found in a cell or tissue from a subject that does not have a PI3Kα-associated disease, e.g., a PI3Kα-associated cancer (and optionally also does not have an increased risk of developing a PI3Kα-associated disease and/or is not suspected of having a PI3Kα-associated disease).

Provided herein is a method of treating cancer (e.g., a PI3Kα-associated cancer) in a subject in need thereof, comprising administering to the subject a therapeutically effective amount of a compound of Formula (I), or a pharmaceutically acceptable salt thereof, or a pharmaceutical composition thereof. For example, provided herein are methods for treating PI3Kα-associated cancer in a subject in need thereof, comprising a) detecting a dysregulation of PIK3CA gene, a PI3Kα protein, or the expression or activity or level of any of the same in a sample from the subject; and b) administering a therapeutically effective amount of a compound of Formula (I), or a pharmaceutically acceptable salt thereof. In some embodiments, the dysregulation of a PIK3CA gene, a PI3Kα protein, or the expression or activity or level of any of the same includes one or more a PI3Kα protein substitutions/point mutations/insertions. Non-limiting examples of PI3Kα protein substitutions/insertions/deletions are described in Table 1.

In some embodiments, the PI3Kα protein substitution/insertion/deletion is selected from the group consisting of E542A, E542G, E542K, E542Q, E542V, E545A, E545D, E545G, E545K, E545Q, M1043I, M1043L, M1043T, M1043V, H1047L, H1047Q, H1047R, H1047Y, G1049R, and combinations thereof. In some embodiments, the PI3Kα protein substitution/insertion/deletion is H1047X, where X is any amino acid other than H. In some embodiments, the PI3Kα protein substitution/insertion/deletion is E542X, where X is any amino acid other than E. In some embodiments, the PI3Kα protein substitution/insertion/deletion is E545X, where X is any amino acid other than E.

In some embodiments, the dysregulation of a PIK3CA gene, a PI3Kα protein, or expression or activity or level of any of the same, includes a splice variation in a PI3Kα mRNA which results in an expressed protein that is an alternatively spliced variant of PI3Kα having at least one residue deleted (as compared to the wild type PI3Kα protein) resulting in a constitutive activity of a PI3Kα protein domain.

In some embodiments, the dysregulation of a PIK3CA gene, a PI3Kα protein, or expression or activity or level of any of the same, includes at least one point mutation in a PIK3CA gene that results in the production of a PI3Kα protein that has one or more amino acid substitutions or insertions or deletions in a PIK3CA gene that results in the production of a PI3Kα protein that has one or more amino acids inserted or removed, as compared to the wild type PI3Kα protein. In some cases, the resulting mutant PI3Kα protein has increased activity, as compared to a wild type PI3Kα protein or a PI3Kα protein not including the same mutation. In some embodiments, the compounds described herein selectively inhibit the resulting mutant PI3Kα protein relative to a wild type PI3Kα protein or a PI3Kα protein not including the same mutation.

In some embodiments of any of the methods or uses described herein, the cancer (e.g., PI3Kα-associated cancer) is selected from a hematological cancer and a solid tumor.

In some embodiments of any of the methods or uses described herein, the cancer (e.g., PI3Kα-associated cancer) is selected from breast cancer (including both HER2⁺ and HER2⁻ breast cancer, ER⁺ breast cancer, and triple negative breast cancer), uterine cancer (including endometrial cancer), lung cancer (including small cell lung cancer (SCLC) and non-small cell lung cancer (NSCLS, including adenocarcinoma lung cancer and squamous cell lung carcinoma)), esophageal squamous cell carcinoma, ovarian cancer, colorectal cancer, esophagastric adenocarcinoma, bladder cancer, head and neck cancer (including head and neck squamous cell cancers such as oropharyngeal squamous cell carcinoma), thyroid cancer, glioma, cervical cancer, lymphangioma, meningioma, melanoma (including uveal melanoma), kidney cancer, pancreatic neuroendocine neoplasms (pNETs), stomach cancer, esophageal cancer, acute myeloid leukemia, relapsed and refractory multiple myeloma, hepatocellular carcinoma, prostate cancer, Malignant Peripheral Nerve Sheath Tumor (MPNST), glioblastoma, cholangiocarcinoma, and pancreatic cancer.

In some embodiments of any of the methods or uses described herein, the cancer (e.g., PI3Kα-associated cancer) is selected from breast cancer (including both HER2⁺ and HER2⁻ breast cancer, ER⁺ breast cancer, and triple negative breast cancer), colon cancer, rectal cancer, colorectal cancer, ovarian cancer, lymphangioma, meningioma, head and neck squamous cell cancer (including oropharyngeal squamous cell carcinoma), melanoma (including uveal melanoma), kidney cancer, pancreatic neuroendocine neoplasms (pNETs), stomach cancer, esophageal cancer, acute myeloid leukemia, relapsed and refractory multiple myeloma, pancreatic cancer, lung cancer (including adenocarcinoma lung cancer and squamous cell lung carcinoma), and endometrial cancer.

In some embodiments of any of the methods or uses described herein, the cancer (e.g., PI3Kα-associated cancer) is selected from breast cancer, SCLC, NSCLC, endometrial cancer, esophageal squamous cell carcinoma, ovarian cancer, colorectal cancer, esophagastric adenocarcinoma, bladder cancer, head and neck cancer, thyroid cancer, glioma, and cervical cancer.

In some embodiments of any of the methods or uses described herein, the PI3Kα-associated cancer is breast cancer. In some embodiments of any of the methods or uses described herein, the PIK3Kα-associated cancer is colorectal cancer. In some embodiments of any of the methods or uses described herein, the PI3Kα-associated cancer is endometrial cancer. In some embodiments of any of the methods or uses described herein, the PI3Kα-associated cancer is lung cancer.

In some embodiments of any of the methods or uses described herein, the PI3Kα-associated cancer is selected from the cancers described in Table 1.

TABLE 1
PI3Kα Protein Amino Acid Substitutions/Insertions/Deletions^{A}

| | Non-Limiting | |
|---|---|---|
| Amino Acid | Exemplary | |
| Position | Mutations | Non-Limiting Exemplary PI3Kα Associated Cancer(s) |
| 1043 | M1043I, M1043L, | Breast Invasive Lobular Carcinoma |
| | M1043T, M1043V | Tubular Stomach Adenocarcinoma |
| | | Uterine Endometrioid Carcinoma |
| | | Mucinous Adenocarcinoma of the Colon and Rectum |
| | | Papillary Thyroid Cancer |
| | | Esophageal Squamous Cell Carcinoma |
| | | Colon Adenocarcinoma |
| | | Breast Invasive Ductal Carcinoma |
| | | Bladder Urothelial Carcinoma |
| | | Pancreatic Adenocarcinoma |
| | | Oligodendroglioma |
| | | Uterine Serous Carcinoma/Uterine Papillary Serous |
| | | Carcinoma |
| | | Glioblastoma Multiforme |
| | | Head and Neck Squamous Cell Carcinoma |
| 1044 | N1044I, N1044K, | Uterine Endometrioid Carcinoma |
| | N1044Y | Breast Invasive Ductal Carcinoma |
| 1045 | D1045A, D1045V | Uterine Endometrioid Carcinoma |
| | | Lung Squamous Cell Carcinoma |
| 1047 | H1047L, H1047Q, | Esophageal Squamous Cell Carcinoma |
| | H1047R, H1047Y | Uterine Endometrioid Carcinoma |
| | | Hepatocellular Carcinoma |
| | | Cutaneous Melanoma |
| | | Mucinous Adenocarcinoma of the Colon and Rectum |
| | | Bladder Urothelial Carcinoma |
| | | Cervical Squamous Cell Carcinoma |
| | | Intrahepatic Cholangiocarcinoma |
| | | Uterine Mixed Endometrial Carcinoma |
| | | Breast Invasive Ductal Carcinoma |
| | | Renal Clear Cell Carcinoma |
| | | Uterine Serous Carcinoma/Uterine Papillary Serous |
| | | Carcinoma |
| | | Head and Neck Squamous Cell Carcinoma |
| | | Lung Squamous Cell Carcinoma |
| | | Breast Invasive Lobular Carcinoma |
| | | Breast Invasive Carcinoma (NOS) |
| | | Astrocytoma |
| | | Colon Adenocarcinoma |
| | | Leiomyosarcoma |
| | | Uterine Carcinosarcoma/Uterine Malignant Mixed |
| | | Mullerian Tumor |
| | | Oligodendroglioma |
| | | Serous Ovarian Cancer |
| | | Mucinous Stomach Adenocarcinoma |
| | | Rectal Adenocarcinoma |
| | | Intestinal Type Stomach Adenocarcinoma |
| | | Diffuse Type Stomach Adenocarcinoma |
| | | Prostate Adenocarcinoma |
| | | Lung Adenocarcinoma |
| | | Stomach Adenocarcinoma |
| | | Tubular Stomach Adenocarcinoma |
| | | Adrenocortical Carcinoma |
| | | Undifferentiated Pleomorphic Sarcoma/Malignant Fibrous |
| | | Histiocytoma/High-Grade Spindle Cell Sarcoma |
| | | Glioblastoma Multiforme |
| | | Oligoastrocytoma |
| 1048 | H1048R | Colon Adenocarcinoma |
| | | Renal Clear Cell Carcinoma |
| 1049 | G1049R | Intestinal Type Stomach Adenocarcinoma |
| | | Bladder Urothelial Carcinoma |
| | | Renal Clear Cell Carcinoma |
| | | Breast Invasive Ductal Carcinoma |
| | | Breast Invasive Lobular Carcinoma |
| | | Uterine Endometrioid Carcinoma |
| | | Colon Adenocarcinoma |
| 1052 | T1052K | Hepatocellular Carcinoma |
| | | Colon Adenocarcinoma |
| 1055 | M1055I | Uterine Mixed Endometrial Carcinoma |
| 1058 | I1058M | Uterine Carcinosarcoma/Uterine Malignant Mixed |
| | | Mullerian Tumor |
| 1065 | H1065L | Breast Invasive Lobular Carcinoma |
| 1066 | A1066V | Uterine Mixed Endometrial Carcinoma |
| 1068 | N1068Y, | Pleural Mesothelioma, Epithelioid Type |
| | N1068fs*5 | Dedifferentiated Liposarcoma |
| | (Frame Shift | Head and Neck Squamous Cell Carcinoma |
| | Insertion) | |

^{A}Unless noted otherwise, the mutations of Table 1 are found in cBioPortal database derived
from Cerami et al. The cBio Cancer Genomics Portal: An Open Platform for Exploring Multidimensional Cancer Genomics Data. Cancer Discovery. May 2012 2; 401; and Gao et al. Integrative analysis of complex cancer genomics and clinical profiles using the cBioPortal. Sci. Signal. 6, pl1 (2013).
† Velho S, Oliveira C, Ferreira A, Ferreira A C, Suriano G, Schwartz S Jr, Duval A, Carneiro F, Machado J C, Hamelin R, Seruca R. The prevalence of PIK3CA mutations in gastric and colon cancer. Eur J Cancer. 2005 Jul; 41(11): 1649-54. doi: 10.1016/j.ejca.2005.04.022. PMID: 15994075.

Exemplary Sequence of Human Phosphatidylinositol 4,5-bisphosphate 3-kinase isoform alpha (UniProtKB entry P42336)

(SEQ ID NO: 1)
MPPRPSSGEL WGIHLMPPRI LVECLLPNGM IVTLECLREA
TLITIKHELF KEARKYPLHQ LLQDESSYIF VSVTQEAERE
EFFDETRRLC DLRLFQPFLK VIEPVGNREE KILNREIGFA
IGMPVCEFDM VKDPEVQDFR RNILNVCKEA VDLRDLNSPH
SRAMYVYPPN VESSPELPKH IYNKLDKGQI IVVIWVIVSP
NNDKQKYTLK INHDCVPEQV IAEAIRKKTR SMLLSSEQLK
LCVLEYQGKY ILKVCGCDEY FLEKYPLSQY KYIRSCIMLG
RMPNLMLMAK ESLYSQLPMD CFTMPSYSRR ISTATPYMNG
ETSTKSLWVI NSALRIKILC ATYVNVNIRD IDKIYVRTGI
YHGGEPLCDN VNTQRVPCSN PRWNEWLNYD IYIPDLPRAA
RLCLSICSVK GRKGAKEEHC PLAWGNINLF DYTDTLVSGK
MALNLWPVPH GLEDLLNPIG VTGSNPNKET PCLELEFDWF
SSVVKFPDMS VIEEHANWSV SREAGFSYSH AGLSNRLARD
NELRENDKEQ LKAISTRDPL SEITEQEKDF LWSHRHYCVT
IPEILPKLLL SVKWNSRDEV AQMYCLVKDW PPIKPEQAME
LLDCNYPDPM VRGFAVRCLE KYLTDDKLSQ YLIQLVQVLK
YEQYLDNLLV RFLLKKALTN QRIGHFFFWH LKSEMHNKTV
SQRFGLLLES YCRACGMYLK HLNRQVEAME KLINLTDILK
QEKKDETQKV QMKFLVEQMR RPDFMDALQG FLSPLNPAHQ
LGNLRLEECR IMSSAKRPLW LNWENPDIMS ELLFQNNEII
FKNGDDLRQD MLTLQIIRIM ENIWQNQGLD LRMLPYGCLS
IGDCVGLIEV VRNSHTIMQI QCKGGLKGAL QFNSHTLHQW
LKDKNKGEIY DAAIDLFTRS CAGYCVATFI LGIGDRHNSN
IMVKDDGQLF HIDFGHFLDH KKKKFGYKRE RVPFVLTQDF
LIVISKGAQE CTKTREFERF QEMCYKAYLA IRQHANLFIN
LFSMMLGSGM PELQSFDDIA YIRKTLALDK TEQEALEYFM
KQMNDAHHGG WTTKMDWIFH TIKQHALN

Also provided is a method for inhibiting PI3Kα activity in a cell, comprising contacting the cell with a compound of Formula (I), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof, to a subject having a cell having aberrant PI3Kα activity. In some embodiments, the cell is a cancer cell. In some embodiments, the cancer cell is any cancer as described herein. In some embodiments, the cancer cell is a PI3Kα-associated cancer cell. As used herein, the term “contacting” refers to the bringing together of indicated moieties in an in vitro system or an in vivo system. For example, “contacting” a PI3Kα protein with a compound provided herein includes the administration of a compound provided herein to an individual or subject, such as a human, having a PI3Kα protein, as well as, for example, introducing a compound provided herein into a sample containing a cellular or purified preparation containing the PI3Kα protein.

Also provided herein is a method of inhibiting cell proliferation, in vitro or in vivo, comprising contacting a cell with an effective amount of a compound of Formula (I), or a pharmaceutically acceptable salt thereof, or a pharmaceutical composition thereof as defined herein.

Further provided herein is a method of increase cell death, in vitro or in vivo, comprising contacting a cell with an effective amount of a compound of Formula (I), or a pharmaceutically acceptable salt thereof, or a pharmaceutical composition thereof as defined herein. Also provided herein is a method of increasing tumor cell death in a subject, comprising administering to the subject an effective compound of Formula (I), or a pharmaceutically acceptable salt thereof, in an amount effective to increase tumor cell death.

In some embodiments, the PI3Kα is human PI3Kα. In some embodiments, the PI3Kα has one or more point mutations in the PIK3CA gene. In some embodiments, the point mutations include a substitution at amino acid position 1047 of a human PI3Kα protein. In some embodiments, the substitution is H1047R.

When employed as pharmaceuticals, the compounds of Formula (I), including pharmaceutically acceptable salts thereof, can be administered in the form of pharmaceutical compositions as described herein.

### Embodiments

1. A compound of Formula (I):

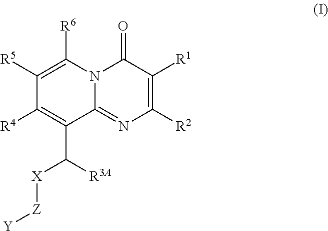

or a pharmaceutically acceptable salt thereof, wherein:
   R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
   R² is phenyl optionally substituted with 1-3 independently selected R^{2A}, 5-10 membered heteroaryl optionally substituted with 1-3 independently selected R^{2A}, 4-10 membered heterocyclyl optionally substituted with 1-3 independently selected R^{2A}, 4-10 membered cycloalkyl optionally substituted with 1-3 independently selected R^{2A}, C1-C6 alkoxyalkyl optionally substituted with —C(═O)NR^{A}R^{C}, C1-C6 alkoxy optionally substituted with —C(═O)NR^{A}R^{C}, or —O(R^{2B});
   each R^{2A} is independently selected from:
   (i) halogen,
   (ii) cyano,
   (iii) hydroxyl,
   (iv) —NR^{A}R^{B},
   (v) —C(═O)NR^{A}R^{B},

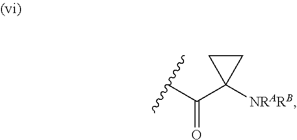

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B}, or C1-C6 alkyl optionally substituted with C1-C6 alkoxy or —NHC(═O)C1-C6 alkyl optionally substituted with C3-C16 cycloalkyl,
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
R^{2B} is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A};
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂,

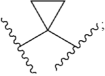 [SMILES: *C1(*)CC1]

Z is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(=NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

2. The compound of Embodiment 1, wherein R¹ is hydrogen.

3. The compound of Embodiment 1, wherein R¹ cyano.

4. The compound of Embodiment 1, wherein R¹ is C3-C6 cycloalkyl.

5. The compound of Embodiment 1, wherein R¹ is C1-C6 alkyl optionally substituted with phenyl optionally substituted with halogen.

6. The compound of Embodiment 1 or 5, wherein R¹ is C1-C6 alkyl substituted with phenyl optionally substituted with halogen.

7. The compound of Embodiment 1 or 5-6, wherein R¹ is C1-C6 alkyl substituted with phenyl substituted with halogen.

8. The compound of Embodiment 1 or 5-6, wherein R¹ is C1-C6 alkyl substituted with phenyl.

9. The compound of Embodiment 1 or 5, wherein R¹ is C1-C6 alkyl.

10. The compound of Embodiment 1, wherein R¹ is C1-C6 thioalkyl.

11. The compound of Embodiment 1, wherein R¹ is C1-C6 haloalkyl.

12. The compound of Embodiment 1, wherein R¹ is C1-C6 alkoxy.

13. The compound of Embodiment 1, wherein R¹ is C1-C6 alkoxyalkyl.

14. The compound of any one of Embodiments 1-13, wherein R² is phenyl optionally substituted with 1-3 independently selected R^{2A}.

15. The compound of any one of Embodiments 1-13, wherein R² is 5-10 membered heteroaryl optionally substituted with 1-3 independently selected R^{2A}.

16. The compound of any one of Embodiments 1-13, wherein R² is 4-10 membered heterocyclyl optionally substituted with 1-3 independently selected R^{2A}.

17. The compound of any one of Embodiments 1-13, wherein R² is 4-10 membered cycloalkyl optionally substituted with 1-3 independently selected R^{2A} or —O(R^{2B}).

18. The compound of any one of Embodiments 1-17, wherein 1, 2, 3, or 4 of R^{2A} are independently halogen.

19. The compound of any one of Embodiments 1-18, wherein 1, 2, 3, or 4 of R^{2A} are independently cyano.

20. The compound of any one of Embodiments 1-19, wherein 1, 2, 3, or 4 of R^{2A} are independently hydroxyl.

21. The compound of any one of Embodiments 1-20, wherein 1, 2, 3, or 4 of R^{2A} are independently —NR^{A}R^{B}.

22. The compound of any one of Embodiments 1-21, wherein 1, 2, 3, or 4 of R^{2A} are independently —C(═O)NR^{A}R^{B}.

23. The compound of any one of Embodiments 1-22, wherein 1, 2, 3, or 4 of R^{2A} are independently

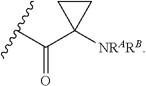

24. The compound of any one of Embodiments 1-23, wherein 1, 2, 3, or 4 of R^{2A} are independently —NHC(═O)R^{C}.

25. The compound of any one of Embodiments 1-24, wherein 1, 2, 3, or 4 of R^{2A} are independently —C(═O)NR^{D}R^{E}.

26. The compound of any one of Embodiments 1-25, wherein 1, 2, 3, or 4 of R^{2A} are independently —C(═O)OR^{F}.

27. The compound of any one of Embodiments 1-26, wherein 1, 2, 3, or 4 of R^{2A} are independently —SO₂R^{F}.

28. The compound of any one of Embodiments 1-27, wherein 1, 2, 3, or 4 of R^{2A} are independently —NHSO₂R^{F}.

29. The compound of any one of Embodiments 1-28, wherein 1, 2, 3, or 4 of R^{2A} are independently —SO₂NR^{F}R^{G}.

30. The compound of any one of Embodiments 1-29, wherein 1, 2, 3, or 4 of R^{2A} are independently —NHC(═O)C1-C6 alkyl optionally substituted with NR^{A}R^{B}.

31. The compound of any one of Embodiments 1-30, wherein 1, 2, 3, or 4 of R^{2A} are independently —NHC(═O)C1-C6 alkyl substituted with NR^{A}R^{B}.

32. The compound of any one of Embodiments 1-31, wherein 1, 2, 3, or 4 of R^{2A} are independently C1-C6 haloalkyl.

33. The compound of any one of Embodiments 1-32, wherein 1, 2, 3, or 4 of R^{2A} are independently C1-C6 hydroxyalkyl.

34. The compound of any one of Embodiments 1-33, wherein 1, 2, 3, or 4 of R^{2A} are independently 5-10 membered heteroaryl optionally substituted with 1-3 substituents independently selected from C1-C6 alkyl and —NR^{A}R^{B}.

35. The compound of any one of Embodiments 1-34, wherein 1, 2, 3, or 4 of R^{2A} are independently 5-10 membered heteroaryl substituted with 1-3 substituents independently selected from C1-C6 alkyl and —NR^{A}R^{B}.

36. The compound of any one of Embodiments 1-35, wherein 1, 2, 3, or 4 of R^{2A} are independently 5-10 membered heteroaryl.

37. The compound of any one of Embodiments 1-36, wherein 1, 2, 3, or 4 of R^{2A} are independently 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

38. The compound of any one of Embodiments 1-37, wherein 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

39. The compound of any one of Embodiments 1-38, wherein 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl substituted with C1-C6 alkoxy, —C(═O)NR^{A}R^{B}, or —NHC(═O)C1-C6 alkyl substituted with —NR^{A}R^{B}.

40. The compound of any one of Embodiments 1-39, wherein 1, 2, 3, or 4 of R^{2A} are independently 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B} or C1-C6 alkyl optionally substituted with C1-C6 alkoxy or —NHC(═O)C1-C6 alkyl optionally substituted with C3-C16 cycloalkyl.

41. The compound of any one of Embodiments 1-36, wherein 1, 2, 3, or 4 of R^{2A} are independently 4-10 membered heterocyclyl.

42. The compound of any one of Embodiments 1-41, wherein 1, 2, 3, or 4 of R^{2A} are independently C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

43. The compound of any one of Embodiments 1-42, wherein 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

44. The compound of any one of Embodiments 1-43, wherein 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, and 4-10 membered heterocyclyl substituted with hydroxyl, C1-C6 alkyl, aralkyl, heteroaralkyl, —C(═O)NR^{A}R^{B}, or —C(═O)C3-C6 cycloalkyl.

45. The compound of any one of Embodiments 1-43, wherein 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, and 4-10 membered heterocyclyl.

46. The compound of any one of Embodiments 1-43, wherein 1, 2, 3, or 4 of R^{2A} are independently C1-C6 alkyl.

47. The compound of any one of Embodiments 1-46, wherein 1, 2, 3, or 4 of R^{2A} are independently C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl.

48. The compound of any one of Embodiments 1-47, wherein 1, 2, 3, or 4 of R^{2A} are independently C1-C6 alkoxy substituted with —NR^{A}R^{B} or 4-10 membered heterocyclyl optionally substituted with C1-C6 alkyl, aralkyl, heteroaralkyl, or —C(═O)C3-C6 cycloalkyl.

49. The compound of any one of Embodiments 1-48, wherein 1, 2, 3, or 4 of R^{2A} are independently C1-C6 alkoxy substituted with —NR^{A}R^{B} or 4-10 membered heterocyclyl substituted with C1-C6 alkyl, aralkyl, heteroaralkyl, or —C(═O)C3-C6 cycloalkyl.

50. The compound of any one of Embodiments 1-48, wherein 1, 2, 3, or 4 of R^{2A} are independently C1-C6 alkoxy substituted with —NR^{A}R^{B} or 4-10 membered heterocyclyl.

51. The compound of any one of Embodiments 1-47, wherein 1, 2, 3, or 4 of R^{2A} are independently C1-C6 alkoxy.

52. The compound of any one of Embodiments 1-51, wherein 1, 2, 3, or 4 of R^{2A} are independently C3-C6 cycloalkyl optionally substituted with 4-10 membered heterocyclyl optionally substituted with C1-C6 alkyl.

53. The compound of any one of Embodiments 1-52, wherein 1, 2, 3, or 4 of R^{2A} are independently C3-C6 cycloalkyl substituted with 4-10 membered heterocyclyl optionally substituted with C1-C6 alkyl.

54. The compound of any one of Embodiments 1-53, wherein 1, 2, 3, or 4 of R^{2A} are independently C3-C6 cycloalkyl substituted with 4-10 membered heterocyclyl substituted with C1-C6 alkyl.

55. The compound of any one of Embodiments 1-53, wherein 1, 2, 3, or 4 of R^{2A} are independently C3-C6 cycloalkyl substituted with 4-10 membered heterocyclyl.

56. The compound of any one of Embodiments 1-52, wherein 1, 2, 3, or 4 of R^{2A} are independently C3-C6 cycloalkyl.

57. The compound of any one of Embodiments 1-13, wherein R² is C1-C6 alkoxy optionally substituted with —C(═O)NR^{A}R^{C}.

58. The compound of any one of Embodiments 1-13 or 57, wherein R² is C1-C6 alkoxy substituted with —C(═O)NR^{A}R^{C}.

59. The compound of any one of Embodiments 1-13 or 57, wherein R² is C1-C6 alkoxy.

60. The compound of any one of Embodiments 1-13, R² is C1-C6 alkoxyalkyl optionally substituted with —C(═O)NR^{A}R^{C}.

61. The compound of any one of Embodiments 1-13, R² is C1-C6 alkoxyalkyl.

62. The compound of any one of Embodiments 1-61, wherein X is a bond.

63. The compound of any one of Embodiments 1-61, wherein X is CH₂.

64. The compound of any one of Embodiments 1-61, wherein X is CH(CH₃).

65. The compound of any one of Embodiments 1-61, wherein X is C(CH₃)₂.

66. The compound of any one of Embodiments 1-61, wherein X is

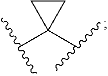 [SMILES: *C1(*)CC1]

67. The compound of any one of Embodiments 1-66, wherein Z is O.

68. The compound of any one of Embodiments 1-66, wherein R^{3A} is hydrogen.

69. The compound of any one of Embodiments 1-66, wherein R^{3A} is C1-C6 alkyl.

70. The compound of any one of Embodiments 1-67, wherein R^{3A} is methyl.

71. The compound of any one of Embodiments 1-66, wherein R^{3A} is C1-C6 alkoxy.

72. The compound of any one of Embodiments 1-66, R^{3A} is C1-C6 haloalkyl.

73. The compound of any one of Embodiments 1-66, wherein Z is NR^{3B}.

74. The compound of Embodiment 73, wherein one of R^{3A} and R^{3B} is hydrogen and the other of R^{3A} and R^{3B} is C1-C6 alkyl.

75. The compound of Embodiment 73-74, wherein one of R^{3A} and R^{3B} is hydrogen and the other of R^{3A} and R^{3B} is methyl.

76. The compound of Embodiment 73, wherein each of R^{3A} and R^{3B} is hydrogen.

77. The compound of Embodiment 73, wherein each of R^{3A} and R^{3B} is an independently selected C1-C6 alkyl.

78. The compound of Embodiment 73 or 77, wherein each of R^{3A} and R^{3B} is methyl.

79. The compound of Embodiment 73, wherein one of R^{3A} and R^{3B} is hydrogen and the other of R^{3A} and R^{3B} is C1-C6 alkoxy.

80. The compound of Embodiment 73, wherein one of R^{3A} and R^{3B} is C1-C6 alkyl and the other of R^{3A} and R^{3B} is C1-C6 alkoxy.

81. The compound of Embodiment 73, wherein each of R^{3A} and R^{3B} is C1-C6 alkoxy.

82. The compound of Embodiment 73, wherein one of R^{3A} and R^{3B} is hydrogen and the other of R^{3A} and R^{3B} is C1-C6 haloalkyl.

83. The compound of Embodiment 73, wherein one of R^{3A} and R^{3B} is C1-C6 alkyl and the other of R^{3A} and R^{3B} is C1-C6 haloalkyl.

84. The compound of Embodiment 73, wherein each of R^{3A} and R^{3B} is C1-C6 haloalkyl.

85. The compound of Embodiment 73, wherein R^{3A} and R^{3B}, together with the carbon and nitrogen atoms, respectively, to which they are attached together form a 4-8 membered heterocyclyl group.

86. The compound of any one of Embodiments 1-85, wherein Y is phenyl optionally substituted with 1-3 independently selected R^{Y}.

87. The compound of any one of Embodiments 1-85, wherein Y is naphthyl optionally substituted with 1-3 independently selected R^{Y}.

88. The compound of any one of Embodiments 1-85, wherein Y is 5-10 membered heteroaryl optionally substituted with 1-3 independently selected R^{Y}.

89. The compound of any one of Embodiments 1-88, wherein 1, 2, or 3 of R^{Y} is independently halogen.

90. The compound of any one of Embodiments 1-89, wherein 1, 2, or 3 of R^{Y} is hydroxyl.

91. The compound of any one of Embodiments 1-90, wherein 1, 2, or 3 of R^{Y} is cyano.

92. The compound of any one of Embodiments 1-91, wherein 1, 2, or 3 of R^{Y} is independently C1-C6 haloalkyl.

93. The compound of any one of Embodiments 1-92, wherein 1, 2, or 3 of R^{Y} is independently C1-C6 alkoxy.

94. The compound of any one of Embodiments 1-93, wherein 1, 2, or 3 of R^{Y} is independently C1-C6 haloalkoxy.

95. The compound of any one of Embodiments 1-94, wherein 1, 2, or 3 of R^{Y} is independently C1-C6 hydroxyalkyl.

96. The compound of any one of Embodiments 1-95, wherein 1, 2, or 3 of R^{Y} is independently —NHC(═O)R^{C}.

97. The compound of any one of Embodiments 1-96, wherein 1, 2, or 3 of R^{Y} is independently —C(═O)NHR^{Y1}.

98. The compound of any one of Embodiments 1-97, wherein 1, 2, or 3 of R^{Y} is independently —CO₂R^{A}.

99. The compound of any one of Embodiments 1-98, wherein 1, 2, or 3 of R^{Y} is independently —SO₂NR^{F}R^{G}.

100. The compound of any one of Embodiments 1-99, wherein 1, 2, or 3 of R^{Y} is independently —NHSO₂R^{F}.

101. The compound of any one of Embodiments 1-100, wherein 1, 2, or 3 of R, is independently —S(═O)(=NR^{F})R^{G}.

102. The compound of any one of Embodiments 1-101, wherein 1, 2, or 3 of R^{Y} is independently —SO₂(C1-C6 alkyl).

103. The compound of any one of Embodiments 1-102, wherein 1, 2, or 3 of R^{Y} is independently —C(═O)NR^{A}R^{B}.

104. The compound of any one of Embodiments 1-103, wherein 1, 2, or 3 of R^{Y} is independently 4-6 membered heteroaryl.

105. The compound of any one of Embodiments 1-104, wherein 1, 2, or 3 of R, is independently heteroaralkyl.

106. The compound of any one of Embodiments 1-105, wherein 1, 2, or 3 of R^{Y} is independently 4-6 membered heterocyclyl.

107. The compound of any one of Embodiments 1-106, wherein 1, 2, or 3 of R^{Y} is independently C1-C6 alkyl optionally substituted with —CO₂R^{A} or 5-6 membered heteroaryl optionally substituted with R^{Y1}.

108. The compound of any one of Embodiments 1-107, wherein 1, 2, or 3 of R^{Y} is independently C1-C6 alkyl substituted with —CO₂R^{A} or 5-6 membered heteroaryl optionally substituted with R^{Y1}.

109. The compound of any one of Embodiments 1-108, wherein 1, 2, or 3 of R^{Y} is independently C1-C6 alkyl substituted with —CO₂R^{A} or 5-6 membered heteroaryl substituted with R^{Y1}.

110. The compound of any one of Embodiments 1-107, wherein 1, 2, or 3 of R^{Y} is independently C1-C6 alkyl substituted with —CO₂R^{A} or 5-6 membered heteroaryl.

111. The compound of any one of Embodiments 1-107, wherein 1, 2, or 3 of R^{Y} is independently C1-C6 alkyl.

112. The compound of any one of Embodiments 1-88 or 107-109, wherein R^{Y1} is —SO₂(C1-C6 alkyl).

113. The compound of any one of Embodiments 1-88 or 107-109, wherein R^{Y1} is C1-C6 alkyl optionally substituted with oxo.

114. The compound of any one of Embodiments 1-107, wherein 1, 2, or 3 of R^{Y1} is hydroxyl.

115. The compound of any one of Embodiments 1-10114, wherein R⁴ is hydrogen.

116. The compound of any one of Embodiments 1-114, wherein R⁴ is C1-C6 alkyl.

117. The compound of any one of Embodiments 1-114, wherein R⁴ is acrylamido.

118. The compound of any one of Embodiments 1-117, wherein R⁵ is hydrogen.

119. The compound of any one of Embodiments 1-117, wherein R⁵ is C1-C6 alkyl.

120. The compound of any one of Embodiments 1-117, wherein R⁵ is cyano.

121. The compound of any one of Embodiments 1-117, wherein R⁵ is —NR^{5A}R^{5B}.

122. The compound of any one of Embodiments 1-117, wherein R⁵ is —C(═O)NR^{5A}R^{5B}.

123. The compound of any one of Embodiments 1-117, wherein R⁵ is —NR^{5A}C(═O)R^{5B}.

124. The compound of any one of Embodiments 1-117 or 121-123, wherein one of R^{5A} and R^{5B} is hydrogen and the other of R^{5A} and R^{5B} is C1-C6 alkyl, C2-C6 alkenyl, or C1-C6 hydroxyalkyl.

125. The compound of any one of Embodiments 1-117 or 121-123, wherein one of R^{5A} and R^{5B} is C1-C6 alkyl and the other of R^{5A} and R^{5B} is C1-C6 alkyl, C2-C6 alkenyl, or C1-C6 hydroxyalkyl.

126. The compound of any one of Embodiments 1-103 or 121-123, wherein each of R^{5A} and R^{5B} is hydrogen.

127. The compound of any one of Embodiments 1-117 or 121-123, wherein each of R^{5A} and R^{5B} is an independently selected C1-C6 alkyl.

128. The compound of any one of Embodiments 1-127, wherein R⁶ is hydrogen.

129. The compound of any one of Embodiments 1-127, wherein R⁶ is halogen.

130. The compound of any one of Embodiments 1-127, wherein R⁶ is C1-C6 alkyl.

131. The compound of any one of Embodiments 1-130, wherein each of R^{A} and R^{B} are independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy.

132. The compound of any one of Embodiments 1-131, wherein one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl optionally substituted with hydroxyl or C1-C6 alkoxy.

133. The compound of any one of Embodiments 1-132, wherein one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl substituted with hydroxyl or C1-C6 alkoxy.

134. The compound of any one of Embodiments 1-132, wherein one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl.

135. The compound of any one of Embodiments 1-130, wherein R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl.

136. The compound of any one of Embodiments 1-130 or 135, wherein R^{A} and R^{B} together with the nitrogen atom to which they are attached form a 4-10 membered heterocyclyl substituted with 1-2 substituents independently selected from halogen, C1-C6 alkyl, and —C(═O)C1-C6 alkyl.

137. The compound of any one of Embodiments 1-130 or 135, wherein R^{A} and R^{B} together with the nitrogen atom to which they are attached form a 4-10 membered heterocyclyl.

138. The compound of any one of Embodiments 1-125, wherein each R^{C} is independently C3-C6 cycloalkyl, —C(═O)NHR^{Y1}, or a C1-C6 alkyl substituted with —NR^{A}R^{B} or with 4-10 membered heterocyclyl optionally substituted with C1-C6 alkyl or with C1-C6 hydroxylalkyl.

139. The compound of any one of Embodiments 1-138, wherein each R^{C} is independently C3-C6 cycloalkyl, —C(═O)NHR^{Y1}, or a C1-C6 alkyl substituted with —NR^{A}R^{B} or with 4-10 membered heterocyclyl substituted with C1-C6 alkyl or with C1-C6 hydroxylalkyl.

140. The compound of any one of Embodiments 1-139, wherein each R^{C} is independently C3-C6 cycloalkyl, —C(═O)NHR¹, or a C1-C6 alkyl substituted with —NR^{A}R^{B} or with 4-10 membered heterocyclyl.

141. The compound of any one of Embodiments 1-138, wherein each R^{C} is independently C3-C6 cycloalkyl, —C(═O)NHR^{Y1}, or a C1-C6 alkyl.

142. The compound of any one of Embodiments 1-141, wherein one of R^{D} and R^{E} is hydrogen and the other of R^{D} and R^{E} is hydroxyl, C1-C6 alkyl, or C1-C6 alkoxy.

143. The compound of any one of Embodiments 1-141, wherein each of R^{D} and R^{E} is hydrogen.

144. The compound of any one of Embodiments 1-141, wherein each of R^{D} and R^{E} is an independently selected C1-C6 alkyl.

145. The compound of any one of Embodiments 1-144, wherein one of R^{F} and R^{G} is hydrogen and the other of R^{F} and R^{G} is phenyl or C1-C6 alkyl optionally substituted with oxo or —NR^{A}R^{B}.

146. The compound of any one of Embodiments 1-144, wherein one of R^{F} and R^{G} is hydrogen and the other of R^{F} and R^{G} is phenyl or C1-C6 alkyl substituted with oxo or —NR^{A}R^{B}.

147. The compound of any one of Embodiments 1-144, wherein one of R^{F} and R^{G} is hydrogen and the other of R^{F} and R^{G} is phenyl or C1-C6 alkyl.

148. The compound of any one of Embodiments 1-144, wherein each of R^{F} and R^{G} is hydrogen.

149. The compound of any one of Embodiments 1-144, wherein each of R^{F} and R^{G} is an independently selected C1-C6 alkyl.

150. A compound selected from the group consisting of the compounds in Table A, or a pharmaceutically acceptable salt thereof.

151. A pharmaceutical composition comprising a compound of any one of Embodiments 1-150, or a pharmaceutically acceptable salt thereof, and one or more pharmaceutically acceptable excipients.

152. A method for treating cancer in a subject in need thereof, comprising administering to the subject a therapeutically effective amount of a compound of any one of Embodiments 1-150, or a pharmaceutically acceptable salt thereof, or a pharmaceutical composition of Embodiment 151.

153. A method for treating cancer in a subject in need thereof, the method comprising (a) determining that the cancer is associated with a dysregulation of a PIK3CA gene, a PI3Kα protein, or expression or activity or level of any of the same; and (b) administering to the subject a therapeutically effective amount of a compound of any one of Embodiments 1-150, or a pharmaceutically acceptable salt thereof, or a pharmaceutical composition of Embodiment 151.

154. A method of treating a PI3Kα-associated cancer in a subject, comprising administering to a subject identified or diagnosed as having a PI3Kα-associated cancer a therapeutically effective amount of a compound of any one of Embodiments 1-150 or a pharmaceutically acceptable salt thereof, or a pharmaceutical composition of Embodiment 151.

155. A method for inhibiting mutant PI3Kα activity in a mammalian cell, the method comprising contacting the mammalian cell with an effective amount of a compound of any one of Embodiments 1-150, or a pharmaceutically acceptable salt thereof.

### EXAMPLES

### Compound Preparation

The general methods for the preparation of the compounds of Formula (I) have been described in an illustrative manner and is intended to be description, rather than of limitation. Thus, it will be appreciated that conditions such as choice of solvent, temperature of reaction, volumes, reaction time may vary while still producing the desired compounds. In addition, it will be appreciated that many of the reagents provided in the following examples may be substituted with other suitable reagents. See, e.g., Smith & March, Advanced Organic Chemistry, 7th Ed. (2013). Such changes and modifications, including without limitation, those relating to the chemical structures, substituents, derivatives, intermediates, syntheses, formulations and/or methods of use provided herein, may be made without departing from the spirit and scope thereof.

The starting materials used for the syntheses are either synthesized or obtained from commercial sources, such as, but not limited to, Sigma-Aldrich, Fluka, Acros Organics, Alfa Aesar, Enamine, Strem, VWR Scientific, and the like. Nuclear Magnetic Resonance (NMR) analysis was conducted using a Bruker AVANCE III HD (300 or 400) MHz spectrometer or Bruker AVANCE NEO 400 MHz spectrometer with an appropriate deuterated solvent. LCMS spectra were obtained on a Shimadzu LCMS-2020 with electrospray ionization in positive ion detection mode with 20ADXR pump, SIL-20ACXR autosampler, CTO-20AC column oven, M20A PDA Detector and LCMS 2020 MS detector.

### Example 1: Preparation of rel-(R)-2-((1-(7-methyl-4-oxo-2-(pyridin-4-yl)-4H-pyrido[1,2-a]pyrimidin-9-yl)ethyl)amino)benzoic acid (Compound 268) and rel-(R)-2-((1-(7-methyl-4-oxo-2-(pyridin-4-yl)-4H-pyrido[1,2-a]pyrimidin-9-yl)ethyl)amino)benzoic acid (Compound 269)

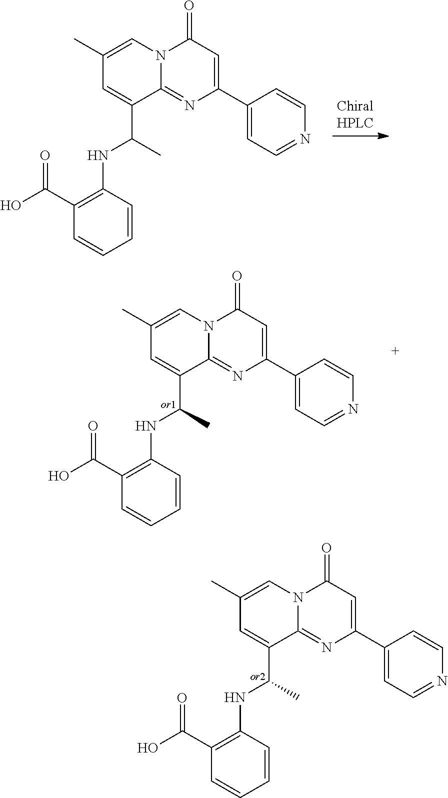 [SMILES: Cc1cc(C(C)Nc2ccccc2C(=O)O)c2nc(-c3ccncc3)cc(=O)n2c1 ; Cc1cc([C@@H](C)Nc2ccccc2C(=O)O)c2nc(-c3ccncc3)cc(=O)n2c1 ; Cc1cc([C@H](C)Nc2ccccc2C(=O)O)c2nc(-c3ccncc3)cc(=O)n2c1]

### Step 1: Preparation of rel-(R)-2-((1-(7-methyl-4-oxo-2-(pyridin-4-yl)-4H-pyrido[1,2-a]pyrimidin-9-yl)ethyl)amino)benzoic acid (Compound 268) and rel-(R)-2-((1-(7-methyl-4-oxo-2-(pyridin-4-yl)-4H-pyrido[1,2-a]pyrimidin-9-yl)ethyl)amino)benzoic acid (Compound 269)

2-((1-(7-methyl-4-oxo-2-(pyridin-4-yl)-4H-pyrido[1,2-a]pyrimidin-9-yl)ethyl)amino)benzoic acid (70 mg) was isolated by Prep-chiral-HPLC with the following conditions: Column: Chiral ART Cellulose-SA, 2*25 cm, 5 μm; Mobile Phase A: Hex (0.1% FA)-HPLC, Mobile Phase B: EtOH-HPLC; Flow rate: 20 mL/min; Gradient: 50% B to 50% B (isocratic) in 12 min; Wave Length: 205/254 nm; RT1 (min): 5.37; RT2 (min): 8.8; Sample Solvent: MeOH:DCM=1:1-HPLC; Injection Volume: 0.8 mL; Number Of Runs: 5. The 1^{st} eluting fraction was concentrated and lyophilized to afford rel-(R)-2-((1-(7-methyl-4-oxo-2-(pyridin-4-yl)-4H-pyrido[1,2-a]pyrimidin-9-yl)ethyl)amino)benzoic acid (Compound 268; 21.9 mg, 31.28%, ee=99.86%) as a white solid, and the 2^{nd} eluting fraction was concentrated and lyophilized to afford rel-(R)-2-((1-(7-methyl-4-oxo-2-(pyridin-4-yl)-4H-pyrido[1,2-a]pyrimidin-9-yl)ethyl)amino)benzoic acid (Compound 269; 19.8 mg, 28.28%, ee=99.71%) as a white solid.

Compound 268: ¹H NMR (300 MHz, DMSO-d₆) δ 8.86-8.67 (m, 3H), 8.57 (s, 1H), 8.28-8.16 (m, 2H), 7.88-7.77 (m, 2H), 7.28-7.11 (m, 2H), 6.61-6.49 (m, 1H), 6.44 (d, J=8.5 Hz, 1H), 5.57 (d, J=7.0 Hz, 1H), 2.37 (s, 3H), 1.69 (d, J=6.6 Hz, 3H). LC-MS: (ES+H, m/z): [M+H]⁺=401.1

Compound 269: ¹H NMR (300 MHz, DMSO-d₆) δ 8.89-8.71 (m, 3H), 8.53 (d, J=6.4 Hz, 1H), 8.26-8.14 (m, 2H), 7.90-7.76 (m, 2H), 7.27-7.10 (m, 2H), 6.55 (t, J=7.6 Hz, 1H), 6.44 (d, J=8.5 Hz, 1H), 5.64-5.47 (m, 1H), 2.37 (s, 3H), 1.70 (d, J=6.6 Hz, 3H). LC-MS: (ES+H, m/z): [M+H]⁺=401.1

### Example 2: Preparation of rel-2-{[(1R)-1-(2-{4-[(4-cyclopropanecarbonylpiperazin-1-yl)methyl]-3-fluorophenyl}-7-methyl-4-oxopyrido[1,2-a]pyrimidin-9-yl)ethyl]amino}benzoic acid (Compound 420) and rel-2-{[(1R)-1-(2-{4-[(4-cyclopropanecarbonylpiperazin-1-yl)methyl]-3-fluorophenyl}-7-methyl-4-oxopyrido[1,2-a]pyrimidin-9-yl)ethyl]amino}benzoic acid (Compound 421)

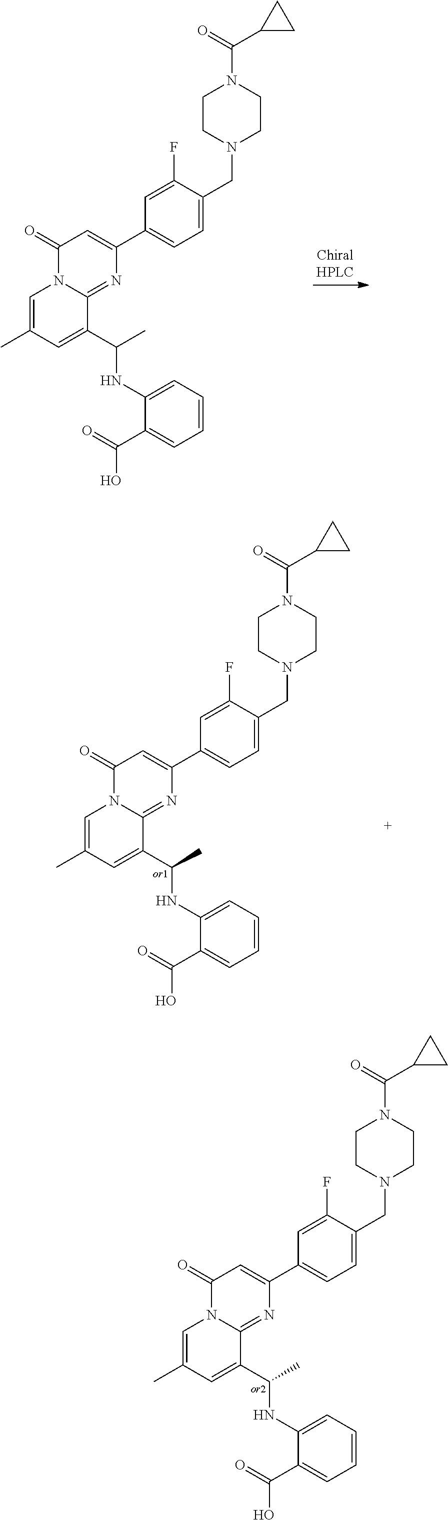 [SMILES: Cc1cc(C(C)Nc2ccccc2C(=O)O)c2nc(-c3ccc(CN4CCN(C(=O)C5CC5)CC4)c(F)c3)cc(=O)n2c1 ; Cc1cc([C@@H](C)Nc2ccccc2C(=O)O)c2nc(-c3ccc(CN4CCN(C(=O)C5CC5)CC4)c(F)c3)cc(=O)n2c1 ; Cc1cc([C@H](C)Nc2ccccc2C(=O)O)c2nc(-c3ccc(CN4CCN(C(=O)C5CC5)CC4)c(F)c3)cc(=O)n2c1]

2-{[1-(2-{4-[(4-cyclopropanecarbonylpiperazin-1-yl)methyl]-3-fluorophenyl}-7-methyl-4-oxopyrido[1,2-a]pyrimidin-9-yl)ethyl]amino}benzoic acid (140 mg, 0.240 mmol, 1 equiv) was isolated by prep-chiral-HPLC with the following conditions: Column: CHIRAL ART Cellulose-SB, 3*25 cm, 5 m; Mobile Phase A: CO₂, Mobile Phase B: IPA (0.1% 2M NH₃-MEOH); Flow rate: 100 mL/min; Gradient: isocratic 50% B; Column Temperature (° C.): 35; Back Pressure (bar): 100; Wave Length: 220/278 nm; RT1 (min): 4.85; RT2 (min): 6, 2 (min): Sample Solvent: IPA; Injection Volume: 2 mL; Number Of Runs: 8. The 1^{st} eluting fraction was concentrated and lyophilized to afford (isomer 1) rel-2-{[(1R)-1-(2-{4-[(4-cyclopropanecarbonylpiperazin-1-yl)methyl]-3-fluorophenyl}-7-methyl-4-oxopyrido[1,2-a]pyrimidin-9-yl)ethyl]amino}benzoic acid (Compound 420; 40.8 mg, 29.08%, ee=100%). The 2^{nd} eluting fraction was concentrated and lyophilized to afford (isomer 2) rel-2-{[(1R)-1-(2-{4-[(4-cyclopropanecarbonylpiperazin-1-yl)methyl]-3-fluorophenyl}-7-methyl-4-oxopyrido[1,2-a]pyrimidin-9-yl)ethyl]amino}benzoic acid (Compound 421; 35.1 mg, 24.87%, ee=97.2%)

Compound 420: ¹H NMR (300 MHz, DMSO-d₆) δ 8.75 (s, 2H), 8.17-8.02 (m, 2H), 7.90-7.81 (m, 1H), 7.78 (d, J=2.0 Hz, 1H), 7.58 (t, J=7.8 Hz, 1H), 7.13 (d, J=14.0 Hz, 2H), 6.54 (t, J=7.5 Hz, 1H), 6.38 (d, J=8.4 Hz, 1H), 5.56 (q, J=7.5 Hz, 1H), 3.65 (d, J=12.1 Hz, 4H), 3.47 (s, 2H), 2.49-2.26 (m, 7H), 2.02-1.88 (m, 1H), 1.66 (d, J=6.5 Hz, 3H), 0.87-0.59 (m, 4H). LC-MS: (ES+H, m z): [M+H]⁺=584.2

Compound 421: ¹H NMR (300 MHz, DMSO-d₆) δ 8.74 (s, 2H), 8.19-7.99 (m, 2H), 7.96-7.87 (m, 1H), 7.77 (d, J=2.0 Hz, 1H), 7.57 (t, J=7.8 Hz, 1H), 7.09 (s, 2H), 6.52 (t, J=7.5 Hz, 1H), 6.32 (d, J=8.4 Hz, 1H), 5.54 (q, J=6.4 Hz, 1H), 3.64 (d, J=13.0 Hz, 4H), 3.47 (s, 2H), 2.48-2.22 (m, 7H), 2.00-1.84 (m, 1H), 1.64 (d, J=6.5 Hz, 3H), 0.79-0.61 (m, 4H). LC-MS: (ES+H, m/z): [M+H]⁺=584.2

### Example 3: Preparation of (R)-2-((1-(2-(1,3-dimethyl-1H-indazol-5-yl)-7-methyl-4-oxo-4H-pyrido[1,2-a]pyrimidin-9-yl)ethyl)amino)benzoic acid (Compound 739) and (S)-2-((1-(2-(1,3-dimethyl-1H-indazol-5-yl)-7-methyl-4-oxo-4H-pyrido[1,2-a]pyrimidin-9-yl)ethyl)amino)benzoic acid (Compound 740)

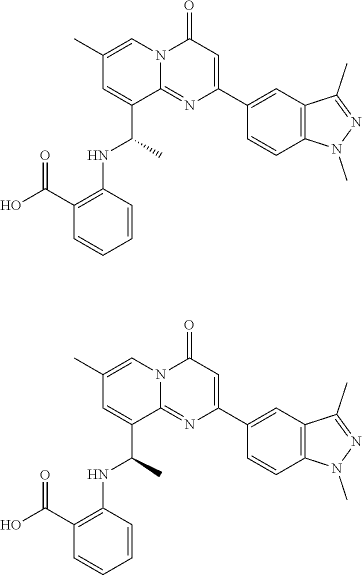 [SMILES: Cc1cc([C@H](C)Nc2ccccc2C(=O)O)c2nc(-c3ccc4c(c3)c(C)nn4C)cc(=O)n2c1 ; Cc1cc([C@@H](C)Nc2ccccc2C(=O)O)c2nc(-c3ccc4c(c3)c(C)nn4C)cc(=O)n2c1]

### Step 1: Synthesis of 9-bromo-2-hydroxy-7-methyl-4H-pyrido[1,2-a]pyrimidin-4-one

A solution of 3-bromo-5-methylpyridin-2-amine (20 g, 108 mmol) in acetone (200 mL) was added bis(2,4,6-trichlorophenyl) malonate (55 g, 119 mmol). The reaction mixture was heated to 60° C. for 2 h under N₂ atmosphere. After cooling to room temperature, the reaction was filtered to give the title compound (19.5 g, 72%) as a yellow solid that required no further purification. ¹H NMR (400 MHz, DMSO-d₆): δ 11.74 (s, 1H), 8.74 (d, J=2.0 Hz, 1H), 8.33 (d, J=2.0 Hz, 1H), 5.50 (s, 1H), 2.34 (s, 3H). MS: m/z 254.8 (M+H⁺).

### Step 2: Synthesis of 2-(benzyloxy)-9-bromo-7-methyl-4H-pyrido[1,2-a]pyrimidin-4-one

A solution of 9-bromo-2-hydroxy-7-methyl-4H-pyrido[1,2-a]pyrimidin-4-one (12.5 g, 49 mmol), K₂CO₃ (13.5 g, 98 mmol) and TBAI (1.8 mg, 4.9 mmol) in DMF (120 mL) was added BnBr (6.71 g, 39.2 mmol). The reaction was heated to 65° C. for 2 h under N₂ atmosphere. After cooling to room temperature, the reaction was added water (200 mL) and extracted with ethyl acetate (200 mL×3). The combined organic layers were washed with brine (200 mL), dried over anhydrous Na₂SO₄, filtered and concentrated. The crude residue was purified by silica gel chromatography (solvent gradient: 0-20% EtOAc in petroleum ether) to give the title compound (4 g, 23%) as a white solid. ¹H NMR (400 MHz, CDCl₃): δ 8.86 (s, 1H), 7.98 (s, 1H), 7.57-7.49 (m, 2H), 7.40-7.30 (m, 3H), 5.85 (s, 1H), 5.52 (s, 2H), 2.40 (s, 3H) MS: m/z 345.0 (M+H⁺).

### Step 3: Synthesis of 9-acetyl-2-(benzyloxy)-7-methyl-4H-pyrido[1,2-a]pyrimidin-4-one

A mixture of 2-(benzyloxy)-9-bromo-7-methyl-4H-pyrido[1,2-a]pyrimidin-4-one (3 g, 8.7 mmol), tributyl(1-ethoxyvinyl)stannane (7.85 g, 21.7 mmol) and Pd(dppf)Cl₂ (509 mg, 0.7 mmol) in dioxane (30 mL) was degassed and purged with N₂ for 3 times. The reaction mixture was stirred at 90° C. for 16 h under N₂ atmosphere. After cooling to room temperature, HCl (3 mL, 1 M) was added. The mixture was stirred at room temperature for 0.5 h. The reaction mixture was added 50 mL 10% KF aqueous solution, stirred at room temperature for 2 h. The mixture was extracted with ethyl acetate (100 mL×3). The combined organic layers were washed with brine (50 mL), dried over anhydrous Na₂SO₄, filtered and concentrated. The crude residue was purified by silica gel chromatography (solvent gradient: 0-50% EtOAc in petroleum ether) to give the title compound (1.39 g, 51%) as a white solid. ¹H NMR (400 MHz, DMSO-d₆): δ 8.94 (s, 1H), 8.07 (s, 1H), 7.47-7.42 (m, 2H), 7.42-7.30 (m, 3H), 5.77 (s, 1H), 5.41 (s, 2H), 2.70 (s, 3H), 2.41 (s, 3H). MS: m/z 309.2 (M+H⁺).

### Step 4: Synthesis of 9-(1-aminoethyl)-2-(benzyloxy)-7-methyl-4H-pyrido[1,2-a]pyrimidin-4-one

To a solution of 9-acetyl-2-(benzyloxy)-7-methyl-4H-pyrido[1,2-a]pyrimidin-4-one (1 g, 3.2 mmol), NH₄OAc (3.75 g, 48.6 mmol) in MeOH (10 mL), NaBH₃CN (610 mg, 9.7 mmol) was added. The reaction was heated to 40° C. for 16 h under N₂ atmosphere. After cooling to room temperature, the reaction was diluted with water (200 mL), extracted with ethyl acetate (300 mL×3). The combined organic layers were washed with brine (200 mL), dried over anhydrous Na₂SO₄, filtered and concentrated in vacuo. The crude residue was purified by silica gel chromatography (solvent gradient: 0-10% MeOH in DCM) to give the title compound (510 mg, 50%) as a white solid. ¹H NMR (400 MHz, DMSO-d₆): δ 8.79 (s, 1H), 8.02 (s, 1H), 7.54-7.26 (m, 5H), 6.42-6.09 (m, 2H), 5.77 (s, 1H), 5.54-5.38 (m, 2H), 4.99-4.75 (m, 1H), 2.41 (s, 3H), 1.45 (d, J=6.8 Hz, 3H). MS: m/z 310.2 (M+H⁺).

### Step 5: Synthesis of tert-butyl 2-((1-(2-(benzyloxy)-7-methyl-4-oxo-4H-pyrido[1,2-a]pyrimidin-9-yl)ethyl)amino)benzoate

A mixture of 9-(1-aminoethyl)-2-(benzyloxy)-7-methyl-4H-pyrido[1,2-a]pyrimidin-4-one (500 mg, 1.6 mmol), tert-butyl 2-bromobenzoate (540 mg, 2.1 mmol), Cs₂CO₃ (1.05 g, 3.2 mmol), Pd₂(dba)₃ (148 mg, 0.16 mmol) and Xantphos (140 mg, 0.24 mmol) in dioxane (8 mL), the reaction mixture was stirred at 100° C. for 16 h under N₂ atmosphere. After cooling to room temperature, the reaction was filtered and the filtrate was concentrated. The crude residue was purified by silica gel chromatography (solvent gradient: 0-20% EtOAc in petroleum ether) to give the title compound (250 mg, 31%) as a white solid. ¹H NMR (400 MHz, DMSO-d₆): δ 8.73 (s, 1H), 8.30-8.15 (m, 1H), 7.80 (d, J=2.0 Hz, 1H), 7.79-7.74 (m, 1H), 7.50-7.42 (m, 2H), 7.41-7.34 (m, 2H), 7.34-7.28 (m, 1H), 7.21-7.13 (m, 1H), 6.61-6.51 (m, 1H), 6.31 (d, J=8.8 Hz, 1H), 5.77 (s, 1H), 5.58-5.45 (m, 2H), 5.33-5.24 (m, 1H), 2.32 (s, 3H), 1.56 (s, 9H), 1.53 (d, J=6.8 Hz, 3H). MS: m/z 486.3 (M+H⁺).

### Step 6: Synthesis of tert-butyl 2-((1-(2-hydroxy-7-methyl-4-oxo-4H-pyrido[1,2-a]pyrimidin-9-yl)ethyl)amino)benzoate

To a solution of tert-butyl 2-((1-(2-(benzyloxy)-7-methyl-4-oxo-4H-pyrido[1,2-a]pyrimidin-9-yl)ethyl)amino)benzoate (2.7 g, 5.6 mmol) in MeOH (30 mL) was added wet Pd/C (3 g, 10% Pd, 50% wet with water). The reaction was stirred at room temperature for 0.5 h under H2 atmosphere (15 psi). The reaction was filtered through diatomaceous earth and the filtrate was concentrated to afford the title compound (1.7 g, 77%) as yellow oil that required no further purification. ¹H NMR (400 MHz, DMSO-d₆): δ 8.74-8.59 (m, 1H), 8.17 (d, J=6.0 Hz, 1H), 7.80-7.74 (m, 1H), 7.71-7.65 (m, 1H), 7.26-7.14 (m, 1H), 6.59-6.50 (m, 1H), 6.27 (d, J=8.4 Hz, 1H), 5.53 (s, 1H), 5.35-5.24 (m, 1H), 2.27 (s, 3H), 1.61-1.49 (m, 12H). MS: m/z 396.2 (M+H⁺).

### Step 7: Synthesis of tert-butyl 2-((1-(7-methyl-4-oxo-2-(((trifluoromethyl)sulfonyl)oxy)-4H-pyrido[1,2-a]pyrimidin-9-yl)ethyl)amino)benzoate

To a solution of tert-butyl 2-((1-(2-hydroxy-7-methyl-4-oxo-4H-pyrido[1,2-a]pyrimidin-9-yl)ethyl)amino)benzoate (500 mg, 1.3 mmol) in DCM (10 mL) was added pyridine (300 mg, 3.8 mmol) and Tf₂O (535 mg, 1.9 mmol). The mixture was stirred at 0° C. for 2 h. After the reaction was completed, the reaction was quenched with water (50 mL) and extracted with ethyl acetate (80 mL×3). The combined organic layers were washed with brine (50 mL), dried over anhydrous Na₂SO₄, filtered and concentrated in vacuo. The crude residue was purified by silica gel chromatography (solvent gradient: 0-10% EtOAc in petroleum ether) to give the title compound (510 mg, 50%) as a white solid. ¹H NMR (400 MHz, DMSO-d₆): δ 8.85-8.22 (m, 1H), 8.21 (d, J=6.6 Hz, 1H), 8.01-7.96 (m, 1H), 7.79-7.78 (m, 1H), 7.19-7.15 (m, 1H), 6.59-6.56 (m, 1H), 6.52 (s, 1H), 6.29 (d, J=8.8 Hz, 1H), 5.23-5.17 (m, 1H), 2.40 (s, 3H), 1.59-1.57 (m, 12H). MS: m/z 528.1 (M+H⁺).

### Step 8: Synthesis of tert-butyl 2-((1-(2-(1,3-dimethyl-1H-indazol-5-yl)-7-methyl-4-oxo-4H-pyrido[1,2-a]pyrimidin-9-yl)ethyl)amino)benzoate

To a mixture of tert-butyl 2-((1-(7-methyl-4-oxo-2-(((trifluoromethyl)sulfonyl)oxy)-4H-pyrido[1,2-a]pyrimidin-9-yl)ethyl)amino)benzoate (300 mg, 569 umol), K₂CO₃ (236 mg, 1.7 mmol) and 1,3-dimethyl-5-(4,4,5,5-tetramethyl-1,3,2-dioxaborolan-2-yl)-1H-indazole (217 mg, 796 umol) (prepared according to the procedure in WO201755305) in dioxane (5 mL) and water (0.5 mL), Pd(PPh₃)₄ (66 mg, 57 umol) was added. The reaction mixture was heated to 100° C. and stirred for 2 h under N₂ atmosphere. The reaction was diluted with water (20 mL) and ethyl acetate (20 mL×3). The combined organic phase was washed with brine (20 mL×2), dried over anhydrous Na₂SO₄, filtered and concentrated. The crude residue was purified by flash chromatography on silica gel eluting with (3% MeOH in DCM) to afford the title compound (290 mg, 97%) as a yellow solid. ¹H NMR (400 MHz, DMSO-d₆): δ 8.76 (s, 1H), 8.70 (s, 1H), 8.27-8.38 (m, 2H), 7.85-7.79 (m, 1H), 7.75-7.66 (m, 1H), 7.23-7.19 (m, 1H), 7.16 (s, 1H), 6.58-6.53 (m, 1H), 6.52-6.48 (m, 1H), 5.60-5.55 (m, 1H), 4.00 (s, 3H), 2.56 (s, 3H), 2.35 (s, 3H), 1.74 (d, J=6.4 Hz, 3H), 1.53 (s, 9H). MS: m/z 524.2 (M+H⁺).

### Step 9: Synthesis of 2-((1-(2-(1,3-dimethyl-1H-indazol-5-yl)-7-methyl-4-oxo-4H-pyrido[1,2-a]pyrimidin-9-yl)ethyl)amino)benzoic acid

To a mixture of tert-butyl 2-((1-(2-(1,3-dimethyl-1H-indazol-5-yl)-7-methyl-4-oxo-4H-pyrido[1,2-a]pyrimidin-9-yl)ethyl)amino)benzoate (50 mg, 95 umol) in DCM (3 mL) was added TFA (0.5 mL, 6.9 mmol). The reaction mixture was stirred at room temperature for 16 h. The mixture was concentrated and the residue was purified by reverse phase chromatography (acetonitrile 51%-81%/0.225% formic acid in water) to give the title compound (5 mg, 11%) as a white solid. ¹H NMR (400 MHz, DMSO-d₆): δ 12.70 (s, 1H), 8.76 (s, 1H), 8.69 (s, 1H), 8.56-8.48 (m, 1H), 8.40-8.32 (m, 1H), 7.86-7.82 (m, 1H), 7.79-7.76 (m, 1H), 7.69-7.65 (m, 1H), 7.24-7.20 (m, 1H), 7.15 (s, 1H), 6.57-6.54 (m, 1H), 6.50-6.47 (m, 1H), 5.63-5.58 (m, 1H), 4.00 (s, 3H), 2.56 (s, 3H), 2.35 (s, 3H), 1.72 (d, J=6.8 Hz, 3H). MS: m/z 468.1 (M+H⁺).

### Step 10: Synthesis of (R)-2-((1-(2-(1,3-dimethyl-1H-indazol-5-yl)-7-methyl-4-oxo-4H-pyrido[1,2-a]pyrimidin-9-yl)ethyl)amino)benzoic acid (Compound 739) and (S)-2-((1-(2-(1,3-dimethyl-1H-indazol-5-yl)-7-methyl-4-oxo-4H-pyrido[1,2-a]pyrimidin-9-yl)ethyl)amino)benzoic acid (Compound 740)

2-((1-(2-(1,3-dimethyl-1H-indazol-5-yl)-7-methyl-4-oxo-4H-pyrido[1,2-a]pyrimidin-9-yl)ethyl)amino)benzoic acid (200 mg, 428 umol) was separated by using chiral SFC (DAICEL CHIRALCEL OJ (250 mm*30 mm, 10 um); Supercritical CO₂/EtOH+0.1% NH₃·H₂O=30/30; 70 mL/min) to afford (R)-2-((1-(2-(1,3-dimethyl-1H-indazol-5-yl)-7-methyl-4-oxo-4H-pyrido[1,2-a]pyrimidin-9-yl)ethyl)amino)benzoic acid (66 mg, first peak) as a white solid and (S)-2-((1-(2-(1,3-dimethyl-1H-indazol-5-yl)-7-methyl-4-oxo-4H-pyrido[1,2-a]pyrimidin-9-yl)ethyl)amino)benzoic acid (61 mg, second peak) as a white solid. Absolute configuration was arbitrarily assigned to each enantiomer.

Compound 740: ¹H NMR (400 MHz, DMSO-d₆): δ 8.75 (s, 1H), 8.69 (s, 1H), 8.35 (d, J=8.8 Hz, 1H), 7.86-7.79 (m, 1H), 7.75 (s, 1H), 7.67 (d, J=8.8 Hz, 1H), 7.21-7.15 (m, 1H), 7.15 (s, 1H), 6.58-6.49 (m, 1H), 6.45-6.40 (m, 1H), 5.61-5.55 (m, 1H), 4.00 (s, 3H), 2.55 (s, 3H), 2.34 (s, 3H), 1.70 (d, 0.1=6.4 Hz, 3H). MS: m/z 468.1 (M+H⁺)

### Example 4: Preparation of 2-(((R)-1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoic acid (Compound 711) and 2-(((S)-1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoic acid (Compound 712)

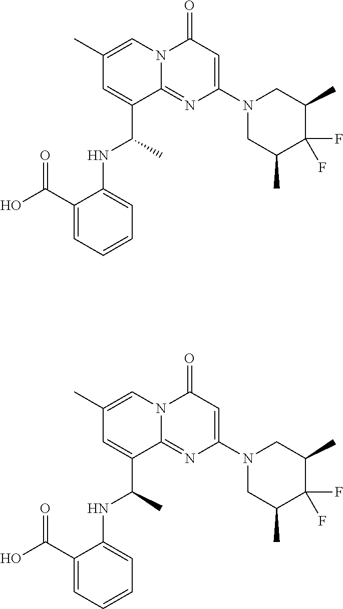 [SMILES: Cc1cc([C@H](C)Nc2ccccc2C(=O)O)c2nc(N3C[C@@H](C)C(F)(F)[C@@H](C)C3)cc(=O)n2c1 ; Cc1cc([C@@H](C)Nc2ccccc2C(=O)O)c2nc(N3C[C@@H](C)C(F)(F)[C@@H](C)C3)cc(=O)n2c1]

### Step 1: Synthesis of tert-butyl 2-((1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoate

To a solution of tert-butyl 2-((1-(2-hydroxy-7-methyl-4-oxo-4H-pyrido[1,2-a]pyrimidin-9-yl)ethyl)amino)benzoate (120 mg, 0.31 mmol) in DMF (5 mL) was added PyBOP (189.5 mg, 0.36 mmol), DIEA (0.16 mL, 0.91 mmol). The mixture was stirred at room temperature for 30 min, cis-4,4-difluoro-3,5-dimethylpiperidine (90 mg, 0.61 mmol, prepared according to the procedure in WO2017125) was added. The reaction was stirred at 40° C. for 16 h. Water (10 mL) was added and extracted with ethyl acetate (50 mL×3). The combined organic layers were washed with aq. HCl (1 M, 80 mL), brine (80 mL), dried over anhydrous Na₂SO₄, filtered and concentrated in vacuo. The crude residue was purified by silica gel chromatography (solvent gradient: 0-20% EtOAc in petroleum ether) to give the title compound (80 mg, 50%) as a white solid. ¹H NMR (400 MHz, DMSO-d₆): δ 8.56 (s, 1H), 8.19 (d, J=5.6 Hz, 1H), 7.80-7.74 (m, 1H), 7.70-7.59 (m, 1H), 7.27-7.21 (m, 1H), 6.57-6.54 (m, 1H), 6.42-6.40 (m, 1H), 5.86 (s, 1H), 5.23-5.20 (m, 1H), 2.88-2.70 (m, 2H), 2.38-2.31 (m, 2H), 2.25 (s, 3H), 2.13-2.01 (m, 2H), 1.61-1.59 (m, 3H), 1.56 (s, 9H), 1.03-0.98 (m, 6H). MS: m/z 527.3 (M+H⁺).

### Step 2: Synthesis of 2-((1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoic acid

To a mixture of tert-butyl 2-((1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoate (140 mg, 0.26 mmol) in DCM (3 mL) was added TFA (1 mL). The mixture was stirred at room temperature for 16 h. This mixture was concentrated in vacuo and then diluted with MeOH (2 mL), then the mixture was adjusted pH to 10 with aq. LiOH (2 M), then adjusted pH to 6 with formic acid. The residue was purified by reverse phase chromatography (acetonitrile 65-95%/0.225% formic acid in water) to give the title compound (80 mg, 63%) as a white solid. ¹H NMR (400 MHz, DMSO-d₆): δ 12.72 (s, 1H), 8.55 (s, 1H), 8.41 (d, J=4.0 Hz, 1H), 7.81 (d, J=4.0 Hz, 1H), 7.61 (s, 1H), 7.24-7.21 (m, 1H), 6.57-6.53 (m, 1H), 6.39-6.37 (m, 1H), 5.86 (s, 1H), 5.25-5.22 (m, 2H), 4.56-4.42 (m, 2H), 2.79-2.73 (m, 2H), 2.24 (s, 3H), 2.11-2.05 (m, 2H), 1.58 (d, J=8.0 Hz, 3H), 1.02-0.99 (m, 6H). MS: m/z 471.2 (M+H⁺).

### Step 3: Synthesis of 2-(((R)-1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoic acid (Compound 711) and 2-(((S)-1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoic acid (Compound 712)

2-((1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoic acid (70 mg, 0.15 mmol) was separated by using chiral SFC (DAICEL CHIRALCEL OD-H (250 mm*30 mm, 5 um); Supercritical CO₂/EtOH+0.1% NH₃·H₂O=65/35; 60 ml/min) to afford 2-(((R)-1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoic acid (Compound 711; 8 mg, first peak) and 2-(((S)-1-(2-((3S,5R)-4,4-difluoro-3,5-dimethylpiperidin-1-yl)-7-methyl-4-oxo-4H-pyrido[1,2-a]pyrimidin-9-yl)ethyl)amino)benzoic acid (Compound 712; 10 mg, second peak) both as white solid. Absolute configuration was arbitrarily assigned to each enantiomer.

Compound 711: ¹H NMR (400 MHz, DMSO-d₆): δ 8.55 (s, 1H), 7.82 (d, J=4.0 Hz, 1H), 7.58 (s, 1H), 7.24-7.10 (m, 1H), 6.57-6.48 (m, 1H), 6.32-6.27 (m, 1H), 5.86 (s, 1H), 5.21-5.19 (m, 2H), 4.52-4.47 (m, 2H), 2.79-2.76 (m, 2H), 2.23 (s, 3H), 2.11-2.04 (m, 2H), 1.55 (d, J=4.0 Hz, 3H), 1.02-0.99 (m, 6H). MS: m/z 471.6 (M+H⁺).

### Example 5: Preparation of 2-((1-(2-(4-fluorophenyl)-3,7-dimethyl-4-oxo-4H-pyrido[1,2-a]pyrimidin-9-yl)ethyl)amino)benzoic acid (Compound 691)

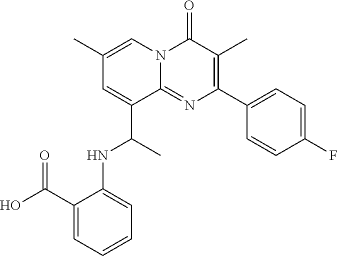 [SMILES: Cc1cc(C(C)Nc2ccccc2C(=O)O)c2nc(-c3ccc(F)cc3)c(C)c(=O)n2c1]

### Step 1: Synthesis of 9-bromo-2-hydroxy-3,7-dimethyl-4H-pyrido[1,2-a]pyrimidin-4-one

To a mixture of 3-bromo-5-methyl-pyridin-2-amine (12.3 g, 65.7 mmol) in acetone (200 mL) was heated to 40° C. for 0.5 h, and then bis(2,4,6-trichlorophenyl) 2-methylpropanedioate (34.5 g, 72.3 mmol) (prepared according to the procedure in Bioorg. Med. Chem. Lett., 2021, 47, 128208) was added dropwise at 40° C. The final mixture was stirred at 65° C. for 4 h. After cooling to room temperature, the reaction mixture was filtrate to give the title compound (15 g, crude) as a yellow solid that required no further purification. ¹H NMR (400 MHz, DMSO-d₆): δ 11.66 (s, 1H), 8.71 (d, J=1.6 Hz, 1H), 8.22 (d, J=1.6 Hz, 1H), 2.34 (s, 3H), 1.96 (s, 3H). MS: m/z 268.8 (M+H⁺).

### Step 2: Synthesis of 2-(benzyloxy)-9-bromo-3,7-dimethyl-4H-pyrido[1,2-a]pyrimidin-4-one

A mixture of 9-bromo-2-hydroxy-3,7-dimethyl-pyrido[1,2-a]pyrimidin-4-one (10 g, 37.2 mmol), K₂CO₃ (10.3 g, 74.3 mmol) and TBAI (1.37 g, 3.72 mmol) in DMF (100 mL) was added (bromomethyl)benzene (3.53 mL, 29.7 mmol). The mixture was stirred at 65° C. for 4 h under N₂ atmosphere. After cooling to room temperature, the reaction mixture was added water (1 L) and yellow solid was precipitated, filtrated. The residue was dissolve in ethyl acetate (1 L), washed with brine (500 mL×3), dried over anhydrous Na₂SO₄, filtered and concentrated under reduced pressure to give the title compound (12.7 g, crude) as a yellow solid that required no further purification. ¹H NMR (400 MHz, DMSO-d₆): δ 8.74 (d, J=1.6 Hz, 1H), 8.28 (s, J=1.6 Hz, 1H), 7.53 (d, J=7.2 Hz, 2H), 7.29-7.41 (m, 3H), 5.55 (s, 2H), 2.37 (s, 3H), 2.02 (s, 3H) MS: m/z 359.0 (M+H⁺).

### Step 3: Synthesis of 9-acetyl-2-(benzyloxy)-3,7-dimethyl-4H-pyrido[1,2-a]pyrimidin-4-one

A mixture of 2-(benzyloxy)-9-bromo-3,7-dimethyl-4H-pyrido[1,2-a]pyrimidin-4-one (20 g, 55.7 mmol) and Pd(dppf)Cl₂ (2.04 g, 2.78 mmol) in dioxane (200 mL) was degassed and purged with N₂ for 3 times, and then tributyl(1-ethoxyvinyl)stannane (40.2 g, 111 mmol) was added, the final mixture was stirred at 90° C. for 16 h under N₂ atmosphere. After cooling to room temperature, HCl (1 M, 20 mL) was added, the mixture was stirred at room temperature for 0.5 h. To the mixture was added 10% KF solution (300 mL), stirred at room temperature for 2 h. The mixture was filtered and extracted with EtOAc (300 mL×3). The combined organic phase was washed with brine (200 mL×2), dried over Na₂SO₄, filtered and concentrated in vacuo. The crude residue was purified with silica gel chromatography (solvent gradient: 0-100% EtOAc in petroleum ether) to give the title compound (13.5 g, 75%) as a yellow solid. ¹H NMR (400 MHz, DMSO-d₆): δ 8.90 (d, J=2.0 Hz, 1H), 8.00 (d, J=2.0 Hz, 1H), 7.45-7.32 (m, 5H), 5.48 (s, 2H), 2.71 (s, 3H), 2.40 (s, 3H), 2.05 (s, 3H). MS: m/z 323.0 (M+H⁺).

### Step 4: Synthesis of 9-(1-aminoethyl)-2-(benzyloxy)-3,7-dimethyl-4H-pyrido[1,2-a]pyrimidin-4-one

A mixture of 9-acetyl-2-benzyloxy-3,7-dimethyl-pyrido[1,2-a]pyrimidin-4-one (8 g, 24.8 mmol), NH₄OAc (28.7 g, 372 mmol), NaBH₃CN (4.68 g, 74.45 mmol) and AcOH (1.42 mL, 24.82 mmol) in MeOH (100 mL) was degassed and purged with N₂ for 3 times, and then the mixture was stirred at 50° C. for 4 h under N₂ atmosphere. After cooling to room temperature, MeOH (100 mL) was removed in vacuo, EtOAc (300 mL) was added, and the mixture was washed with sat. aq. NaHCO₃ (200 mL). The organic layer was dried over Na₂SO₄, filtered and concentrated in vacuo. The crude residue was purified silica gel chromatography (solvent gradient: 0-10% MeOH in DCM) to give the title compound (5 g, 62%) as yellow oil. ¹H NMR (400 MHz, DMSO-d₆): δ 8.69 (s, 1H), 7.92 (s, 1H), 7.46-7.44 (m, 2H), 7.40-7.36 (m, 2H), 7.32-7.29 (m, 1H), 5.52 (s, 2H), 4.68-4.63 (m, 3H), 2.39 (s, 3H), 2.05 (s, 3H), 1.29 (d, J=6.4 Hz, 3H). MS: m/z 324.4 (M+H⁺).

### Step 5: Synthesis of tert-butyl 2-((1-(2-(benzyloxy)-3,7-dimethyl-4-oxo-4H-pyrido[1,2-a]pyrimidin-9-yl)ethyl)amino)benzoate

A mixture of 9-(1-aminoethyl)-2-(benzyloxy)-3,7-dimethyl-4H-pyrido[1,2-a]pyrimidin-4-one (15.1 g, 46.7 mmol), tert-butyl 2-bromobenzoate (18.0 g, 70.0 mmol), Cs₂CO₃ (30.4 g, 93.4 mmol), Pd₂(dba)₃ (2.05 g, 2.24 mmol) and Xantphos (2.70 g, 4.67 mmol) in dioxane (200 mL) was degassed and purged with N₂ for 3 times, and then the mixture was stirred at 100° C. for 16 h under N₂ atmosphere. After cooling to room temperature, dioxane was removed in vacuo to give the crude residue. The crude residue was purified with silica gel chromatography (solvent gradient: 0-100% DCM in petroleum ether) to give the title compound (13.9 g, 60%) as yellow oil. ¹H NMR (400 MHz, DMSO-d₆): δ 8.69 (s, 1H), 8.21 (d, J=6.8 Hz, 1H), 7.78-7.72 (m, 1H), 7.47 (d, J=7.2 Hz, 2H), 7.38-7.34 (m, 2H), 7.32-7.26 (m, 1H), 7.15-7.10 (m, 1H), 6.56-6.54 (m, 1H), 6.29 (d, J=8.8 Hz, 1H), 5.63-5.60 (m, 2H), 5.31-5.28 (m, 1H), 2.31 (s, 3H), 2.08 (s, 3H), 1.56 (s, 9H), 1.52 (d, J=6.4 Hz, 3H). MS: m/z 500.3 (M+H⁺).

### Step 6: Synthesis of tert-butyl 2-((1-(2-hydroxy-3,7-dimethyl-4-oxo-4H-pyrido[1,2-a]pyrimidin-9-yl)ethyl)amino)benzoate

A mixture of tert-butyl 2-((1-(2-(benzyloxy)-3,7-dimethyl-4-oxo-4H-pyrido[1,2-a]pyrimidin-9-yl)ethyl)amino)benzoate (5 g, 10.0 mmol), Pd/C (2.72 g, 2.00 mmol, 10% purity) in MeOH (15 mL) was degassed and purged with H2 (15 psi) for 3 times, and then the mixture was stirred at room temperature for 1 h under H2 atmosphere. After the reaction was completed, the reaction mixture was filtrated. The filtrate was concentrated to give the tilte compound (4 g, crude) as a yellow solid that required no further purification. ¹H NMR (400 MHz, DMSO-d₆): δ 11.47 (s, 1H), 8.63 (s, 1H), 8.17 (d, J=6.0 Hz, 1H), 7.78-7.76 (m, 1H), 7.60 (s, 1H), 7.22-7.18 (m, 1H), 6.57-6.53 (m, 1H), 6.27 (d, J=8.4 Hz, 1H), 5.38-5.32 (m, 1H), 2.27 (s, 3H), 1.99 (s, 3H), 1.58 (s, 9H), 1.57 (d, J=6.8 Hz, 3H). MS: m/z 410.2 (M+H⁺).

### Step 7: Synthesis of tert-butyl 2-((1-(3,7-dimethyl-4-oxo-2-(((trifluoromethyl)sulfonyl)oxy)-4H-pyrido[1,2-a]pyrimidin-9-yl)ethyl)amino)benzoate

To a solution of tert-butyl 2-((1-(2-hydroxy-3,7-dimethyl-4-oxo-4H-pyrido[1,2-a]pyrimidin-9-yl)ethyl)amino)benzoate (0.6 g, 1.47 mmol) in DCM (15 mL) was added pyridine (355 uL, 4.40 mmol) and Tf₂O (363 uL, 2.20 mmol). The mixture was stirred at 0° C. for 2 h. The mixture was diluted with DCM (50 mL), washed with 1M HCl (20 mL), brine (20 mL), dried over Na₂SO₄ and filtered. The filtrate was concentrated to give the crude which was purified with silica gel chromatography (solvent gradient: 0-10% EtOAc in petroleum ether) to give the title compound (0.72 g, 90%) as a yellow solid. ¹H NMR (400 MHz, DMSO-d₆): δ 11.46 (s, 1H), 8.61 (s, 1H), 8.16 (d, J=5.6 Hz, 1H), 7.77 (d, J=6.4 Hz, 1H), 7.57 (s, 1H), 7.21-7.17 (m, 1H), 6.56-6.53 (m, 1H), 6.27 (d, J=8.0 Hz, 1H), 5.36-5.33 (m, 1H), 2.26 (s, 3H), 1.98 (s, 3H), 1.59 (s, 9H), 1.56 (d, J=6.4 Hz, 3H). MS: m/z 542.2 (M+H⁺).

### Step 8: Synthesis of tert-butyl 2-((1-(2-(4-fluorophenyl)-3,7-dimethyl-4-oxo-4H-pyrido[1,2-a]pyrimidin-9-yl)ethyl)amino)benzoate

To a solution of tert-butyl 2-((1-(3,7-dimethyl-4-oxo-2-(((trifluoromethyl)sulfonyl)oxy)-4H-pyrido[1,2-a]pyrimidin-9-yl)ethyl)amino)benzoate (100 mg, 0.19 mmol), K₂CO₃ (77 mg, 0.55 mmol) and (4-fluorophenyl)boronic acid (78 mg, 0.55 mmol) in dioxane (3 mL) and water (1 mL) was added Pd(PPh₃)₄ (43 mg, 0.03 mmol). The reaction was stirred at 100° C. for 2 h under N₂ atmosphere. After cooling to room temperature, the reaction was filtered and concentrated in vacuo. The crude residue was purified by silica gel chromatography (solvent gradient: 0-5% MeOH in DCM) to give the title compound (50 mg, 56%) as yellow oil. MS: m/z 488.2 (M+H⁺).

### Step 9: Synthesis of 2-((1-(2-(4-fluorophenyl)-3,7-dimethyl-4-oxo-4H-pyrido[1,2-a]pyrimidin-9-yl)ethyl)amino)benzoic acid (Compound 721)

To a solution of tert-butyl 2-((1-(2-(4-fluorophenyl)-3,7-dimethyl-4-oxo-4H-pyrido[1,2-a]pyrimidin-9-yl)ethyl)amino)benzoate (60 mg, 0.13 mmol) in DCM (3 mL) was added TFA (0.2 mL, 26 mmol). The reaction mixture was stirred at room temperature for 16 h. The solution was adjusted pH to 10 with aq. LiOH (2 M), then adjusted pH to 6 with formic acid. The crude residue was purified by reverse phase chromatography (acetonitrile 65-95%/0.225% formic acid in water) to afford the title compound (15 mg, 28%) as a yellow solid. ¹H NMR (400 MHz, DMSO-d₆) δ 8.70 (s, 1H), 8.61 (s, 1H), 7.82-7.79 (m, 3H), 7.66 (s, 1H), 7.39-7.34 (m, 2H), 7.16-7.13 (m, 1H), 6.53-6.52 (m, 1H), 6.32 (d, J=8.4 Hz, 1H), 5.38-5.39 (m, 1H), 2.33 (s, 3H), 2.24 (s, 3H), 1.59 (d, J=6.4 Hz, 3H). MS: m/z 432.0 (M+H⁺).

### Example 6: Preparation of (R)-2-((1-(2-(isoindolin-2-yl)-7-methyl-4-oxo-4H-pyrido[1,2-a]pyrimidin-9-yl)ethyl)amino)benzenesulfonamide (Compound 605) and (S)-2-((1-(2-(isoindolin-2-yl)-7-methyl-4-oxo-4H-pyrido[1,2-a]pyrimidin-9-yl)ethyl)amino)benzenesulfonamide (Compound 606)

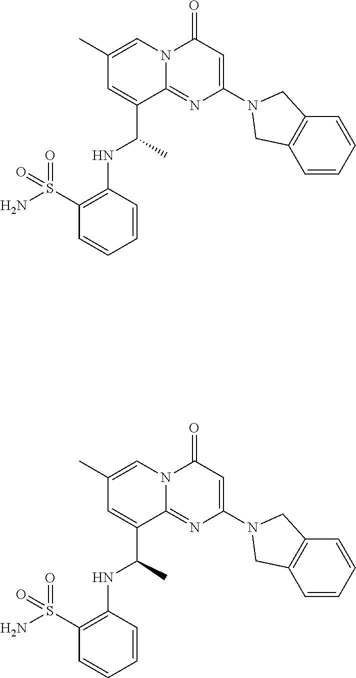 [SMILES: Cc1cc([C@H](C)Nc2ccccc2S(N)(=O)=O)c2nc(N3Cc4ccccc4C3)cc(=O)n2c1 ; Cc1cc([C@@H](C)Nc2ccccc2S(N)(=O)=O)c2nc(N3Cc4ccccc4C3)cc(=O)n2c1]

### Step 1: Synthesis of 2-(benzyloxy)-9-(1-hydroxyethyl)-7-methyl-4H-pyrido[1,2-a]pyrimidin-4-one

To a solution of 9-acetyl-2-benzyloxy-7-methyl-pyrido[1,2-a]pyrimidin-4-one (5 g, 16.2 mmol) in MeOH (50 mL) was added NaBH₄ (950 mg, 25 mmol) at 0° C. The mixture was stirred at 0° C. for 2 h under N₂ atmosphere. The mixture was quenched with sat. aq. NH₄Cl (20 mL), diluted with water (20 mL), extracted with EtOAc (30 mL×2). The combined organic layers were washed with brine (20 mL), dried over anhydrous Na₂SO₄, filtered and concentrated in vacuo. The crude residue was purified by silica gel chromatography (solvent gradient: 0-50% EtOAc in petroleum ether) to give the title compound (3.88 g, 77%) as a yellow solid. MS: m/z 311.2 (M+H⁺).

### Step 2: Synthesis of 2-(benzyloxy)-9-(1-bromoethyl)-7-methyl-4H-pyrido[1,2-a]pyrimidin-4-one

To a solution of 2-benzyloxy-9-(1-hydroxyethyl)-7-methyl-pyrido[1,2-a]pyrimidin-4-one (3.88 g, 12.5 mmol) in DCM (40 mL) was added PBr₃ (6.77 g, 25 mmol) at 0° C. The reaction mixture was stirred at room temperature for 16 h under N₂ atmosphere. The mixture was quenched with sat. aq. NaHCO₃ (30 mL), extracted with EtOAc (50 mL×2). The combined organic layers were washed with brine (20 mL), dried over anhydrous Na₂SO₄, filtered and concentrated in vacuo. The crude residue was purified by silica gel chromatography (solvent gradient: 0-50% EtOAc in petroleum ether) to give the title compound (2.25 g, 48%) as a yellow solid. MS: m/z 375.1 (M+2+H⁻).

### Step 3: Synthesis of 2-((1-(2-(benzyloxy)-7-methyl-4-oxo-4H-pyrido[1,2-a]pyrimidin-9-yl)ethyl)amino)benzenesulfonamide

To a solution of 2-benzyloxy-9-(1-bromoethyl)-7-methyl-pyrido[1,2-a]pyrimidin-4-one (500 mg, 1.34 mmol) in dioxane (6 mL) was added 2-aminobenzenesulfonamide (1.38 g, 8 mmol) at room temperature. The reaction was stirred at 100° C. for 2 h under N₂ atmosphere. After cooling to room temperature, the mixture was concentrated in vacuo. The crude residue was purified by silica gel chromatography (solvent gradient: 0-25% EtOAc in petroleum ether) to give the title compound (370 mg, 59%) as colorless oil. ¹H NMR (400 MHz, DMSO-d₆): δ 8.73 (s, 1H), 7.93 (d, J=1.6 Hz, 1H), 7.64 (dd, J=1.2, 8.0 Hz, 1H), 7.55 (s, 2H), 7.48 (d, J=7.2 Hz, 2H), 7.40-7.35 (m, 2H), 7.33-7.29 (m, 1H), 7.24-7.19 (m, 2H), 7.15 (t, J=7.6 Hz, 1H), 6.79 (d, J=8.0 Hz, 1H), 6.68-6.57 (m, 2H), 6.43 (d, J=6.0 Hz, 1H), 6.28 (d, J=8.4 Hz, 1H), 5.83 (s, 1H), 5.52 (s, 2H), 5.33 (t, J=6.4 Hz, 1H), 3.17 (d, J=5.2 Hz, 2H), 2.30 (s, 3H), 1.52 (d, J=6.4 Hz, 3H). MS: m/z 465.1 (M+H⁺).

### Step 4: Synthesis of 2-((1-(2-hydroxy-7-methyl-4-oxo-4H-pyrido[1,2-a]pyrimidin-9-yl)ethyl)amino)benzenesulfonamide

To a solution of 2-[1-(2-benzyloxy-7-methyl-4-oxo-pyrido[1,2-a]pyrimidin-9-yl)ethylamino]benzenesulfonamide (370 mg, 796 umol) in MeOH (5 mL) was added palladium on carbon (250 mg, 239 umol). The reaction was stirred at room temperature for 2 h under a hydrogen atmosphere. The reaction was filtrated and concentrated in vacuo to give the title compound (160 mg, crude) as a white solid. MS: m/z 375.1 (M+H⁺).

### Step 5: Synthesis of 2-((1-(2-(isoindolin-2-yl)-7-methyl-4-oxo-4H-pyrido[1,2-a]pyrimidin-9-yl)ethyl)amino)benzenesulfonamide

To a solution of 2-[1-(2-hydroxy-7-methyl-4-oxo-pyrido[1,2-a]pyrimidin-9-yl)ethylamino]benzenesulfonamide (100 mg, 267 umol) in DMF (5 mL) was added PyBOP (167 mg, 320 umol), DIEA (0.23 mL, 1.34 mmol). The mixture was stirred at room temperature for 30 min, isoindoline (96 mg, 800 umol) was added. The reaction was stirred at 40° C. for 16 h. Water (10 mL) was added and extracted with ethyl acetate (50 mL×3). The combined organic layers were washed with aq. HCl (1 M, 100 mL) and brine (100 mL), dried over anhydrous Na₂SO₄, filtered and concentrated in vacuo. The residue was purified by reverse phase chromatography (acetonitrile 50-80%/0.225% formic acid in water) to give the title compound (100 mg, 78%) as a yellow solid. MS: m/z 476.0 (M+H⁺).

### Step 6: Synthesis of (R)-2-((1-(2-(isoindolin-2-yl)-7-methyl-4-oxo-4H-pyrido[1,2-a]pyrimidin-9-yl)ethyl)amino)benzenesulfonamide & (S)-2-((1-(2-(isoindolin-2-yl)-7-methyl-4-oxo-4H-pyrido[1,2-a]pyrimidin-9-yl)ethyl)amino)benzenesulfonamide

2-((1-(2-(isoindolin-2-yl)-7-methyl-4-oxo-4H-pyrido[1,2-a]pyrimidin-9-yl)ethyl)amino)benzenesulfonamide (100 mg, 0.21 mmol) was separated by using chiral SFC (DAICEL CHIRALCEL AD (250 mm*30 mm, 10 um); Supercritical CO₂/EtOH+0.1% NH₃·H₂O=35/65; 60 ml/min) to afford (R)-2-((1-(2-(isoindolin-2-yl)-7-methyl-4-oxo-4H-pyrido[1,2-a]pyrimidin-9-yl)ethyl)amino)benzenesulfonamide (Compound 605; 30 mg, first peak) and (S)-2-((1-(2-(isoindolin-2-yl)-7-methyl-4-oxo-4H-pyrido[1,2-a]pyrimidin-9-yl)ethyl)amino)benzenesulfonamide (Compound 606; 30 mg, second peak) both as white solid. Absolute configuration was arbitrarily assigned to each enantiomer.

Compound 605: ¹H NMR (400 MHz, DMSO-d₆): δ 8.59 (s, 1H), 7.78 (s, 1H), 7.64 (d, J=8.0 Hz, 1H), 7.52 (s, 2H), 7.42 (d, J=3.6 Hz, 2H), 7.35-7.31 (m, 2H), 7.23 (t, J=7.6 Hz, 1H), 6.65 (t, J=7.6 Hz, 1H), 6.48-6.44 (m, 2H), 5.44 (s, 1H), 5.42-5.38 (m, 1H), 5.00 (s, 2H), 4.74 (s, 2H), 2.25 (s, 3H), 1.64 (d, J=6.4 Hz, 3H). MS: m/z 476.1 (M+H⁺).

### Example 7: Preparation of 2-(((R)-1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoic acid (Compound 582) and 2-(((S)-1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoic acid (Compound 583)

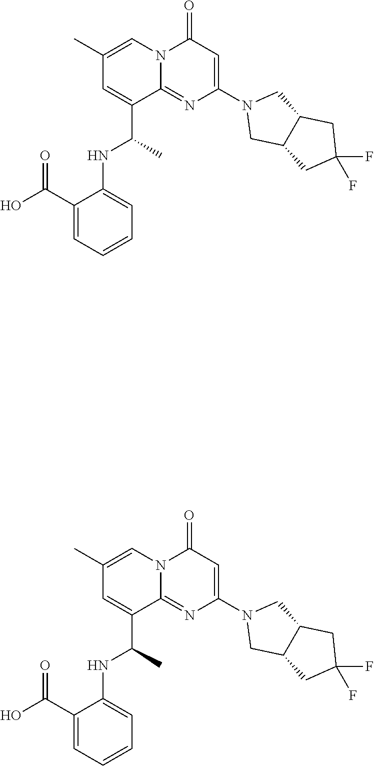 [SMILES: Cc1cc([C@H](C)Nc2ccccc2C(=O)O)c2nc(N3C[C@@H]4CC(F)(F)C[C@@H]4C3)cc(=O)n2c1 ; Cc1cc([C@@H](C)Nc2ccccc2C(=O)O)c2nc(N3C[C@@H]4CC(F)(F)C[C@@H]4C3)cc(=O)n2c1]

### Step 1: Synthesis of tert-butyl (3aR,6aS)-5,5-difluorohexahydrocyclopenta[c]pyrrole-2(1H)-carboxylate

To a solution of tert-butyl (3aR,6aS)-5-oxohexahydrocyclopenta[c]pyrrole-2(1H)-carboxylate (500 mg, 2.22 mmol) in DCM (10 mL) was added DAST (1.43 g, 8.88 mmol) at 0° C. The reaction mixture was stirred at room temperature for 16 h under N₂ atmosphere. The mixture was quenched with sat. aq. NaHCO₃ (10 mL), diluted with water (20 mL), extracted with DCM (30 mL×2). The combined organic layers were washed with brine (20 mL), dried over anhydrous Na₂SO₄, filtered and concentrated in vacuo. The crude residue was purified by silica gel chromatography (solvent gradient: 0-20% EtOAc in petroleum ether) to give the title compound (450 mg, 82%) as a yellow solid. ¹H NMR (400 MHz, DMSO-d₆): δ 3.58-3.43 (m, 2H), 3.19-3.03 (m, 2H), 2.85-2.77 (m, 2H), 2.44-2.25 (m, 2H), 2.11-1.90 (m, 2H), 1.39 (s, 9H).

### Step 2: Synthesis of (3aR,6aS)-5,5-difluorooctahydrocyclopenta[c]pyrrole trifluoroacetate

To a solution of tert-butyl (3aR,6aS)-5,5-difluorohexahydrocyclopenta[c]pyrrole-2(1H)-carboxylate (450 mg, 1.82 mmol) in DCM (10 mL) was added TFA (1.04 g, 9 mmol). The reaction mixture was stirred at room temperature for 2 h under N₂ atmosphere. The mixture was concentrated in vacuo to give the title compound (450 mg, 82%) as a yellow oil. ¹H NMR (400 MHz, DMSO-d₆): δ 3.56-3.42 (m, 2H), 2.76-2.72 (m, 2H), 2.62-2.55 (m, 2H), 2.30-2.13 (m, 2H), 1.92-1.71 (m, 2H).

### Step 3: Synthesis of tert-butyl 2-((1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoate

To a solution of tert-butyl 2-[1-(2-hydroxy-7-methyl-4-oxo-pyrido[1,2-a]pyrimidin-9-yl)ethylamino]benzoate (180 mg, 45 umol) in DMF (5 mL) was added PyBOP (280 mg, 543 umol), DIEA (0.24 mL, 1.81 mmol). The mixture was stirred at room temperature for 30 min, (3aR,6aS)-5,5-difluorooctahydrocyclopenta[c]pyrrole trifluoroacetate (200 mg, 1.36 mmol) was added. The reaction was stirred at 40° C. for 16 h. Water (10 mL) was added and extracted with ethyl acetate (50 mL×3). The combined organic layers were washed with aq. HCl (1 M, 100 mL) and brine (100 mL), dried over anhydrous Na₂SO₄, filtered and concentrated in vacuo. The residue was purified by silica gel chromatography (solvent gradient: 0-50% EtOAc in petroleum ether) to give the title compound (120 mg, 50%) as a yellow solid. ¹H NMR (400 MHz, DMSO-d₆): δ 8.55 (s, 1H), 8.21 (d, J=6.4 Hz, 1H), 7.76 (d, J=5.6, 1H), 7.62 (s, 1H), 7.24 (t, J=7.6 Hz, 1H), 6.55 (t, J=7.2 Hz, 1H), 6.44 (d, J=8.4 Hz, 1H), 5.31 (s, 1H), 5.23 (q, J=6.8 Hz, 1H), 3.95-3.65 (m, 2H), 3.57-3.41 (m, 2H), 3.18-3.15 (m, 1H), 3.01-2.96 (s, 2H), 2.46-2.36 (m, 2H), 2.24 (s, 3H), 2.19-1.99 (m, 2H), 1.62 (d, J=6.8 Hz, 3H), 1.56 (s, 9H). MS: m/z 525.3 (M+H⁺).

### Step 4: 2-((1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoic acid

To a mixture of tert-butyl 2-((1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoate (120 mg, 228 umol) in DCM (3 mL) was added TFA (1 mL). The reaction was stirred at room temperature for 16 h. This mixture was concentrated in vacuo and then dulited with MeOH (2 mL). The mixture was adjusted pH to 10 with aq. LiOH (2 M), then adjusted pH to 6 with formic acid. The residue was purified by reverse phase chromatography (acetonitrile 10-33%/0.225% formic acid in water) to give the title compound (40 mg, 37%) as a white solid. ¹H NMR (400 MHz, DMSO-d₆): δ 12.72 (s, 1H), 8.54 (s, 1H), 8.47 (s, 1H), 7.80 (d, J=7.8 Hz, 1H), 7.58 (d, J=1.6 Hz, 1H), 7.22-7.20 (m, 1H), 6.53 (t, J=7.2 Hz, 1H), 6.36 (d, J=8.4 Hz, 1H), 5.31 (s, 1H), 5.27-5.15 (m, 1H), 3.91-3.64 (m, 2H), 3.54-3.39 (m, 2H), 2.96 (s, 2H), 2.44-2.36 (m, 2H), 2.23 (s, 3H), 2.17-2.04 (m, 2H), 1.59 (d, J=6.4 Hz, 3H). MS: m/z 469.0 (M+H⁺).

### Step 5: Synthesis of 2-(((R)-1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoic acid (Compound 582) and 2-(((S)-1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoic acid (Compound 583)

2-((1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoic acid (40 mg, 85 umol) was separated by using chiral SFC (DAICEL CHIRALCEL OJ (250 mm*30 mm, 10 um); Supercritical CO₂/EtOH+0.1% NH₃·H₂O=30/70; 60 ml/min) to afford 2-(((R)-1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoic acid (15 mg, first peak) and 2-(((S)-1-(2-((3aR,6aS)-5,5-difluorohexahydrocyclopenta[c]pyrrol-2(1H)-yl)-7-methyl-4-oxo-4H-pyrido[1,2-a]pyrimidin-9-yl)ethyl)amino)benzoic acid (11 mg, second peak) both as white solid. Absolute configuration was arbitrarily assigned to each enantiomer.

Compound 583: ¹H NMR (400 MHz, DMSO-d₆): δ 12.70 (s, 1H), 8.54 (s, 1H), 8.50 (s, 1H), 7.80 (d, J=7.6 Hz, 1H), 7.58 (d, J=1.6 Hz, 1H), 7.22-7.20 (m, 1H), 6.54-6.52 (m, 1H), 6.36 (d, J=8.4 Hz, 1H), 5.31 (s, 1H), 5.27-5.15 (m, 1H), 3.91-3.64 (m, 2H), 3.54-3.39 (m, 2H), 2.96 (s, 2H), 2.44-2.36 (m, 2H), 2.23 (s, 3H), 2.17-2.04 (m, 2H), 1.59 (d, J=6.4 Hz, 3H). MS: m/z 469.0 (M+H⁺).

### Example 8: Preparation of (S)-2-((1-(2-(3-amino-1-methyl-1H-indazol-5-yl)-7-methyl-4-oxo-4H-pyrido[1,2-a]pyrimidin-9-yl)ethyl)amino)benzoic acid (Compound 536)

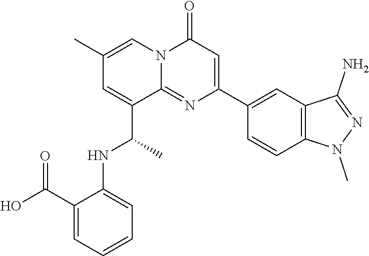 [SMILES: Cc1cc([C@H](C)Nc2ccccc2C(=O)O)c2nc(-c3ccc4c(c3)c(N)nn4C)cc(=O)n2c1]

### Step 1: Synthesis of tert-butyl 2-((1-(2-(3-amino-1-methyl-1H-indazol-5-yl)-7-methyl-4-oxo-4H-pyrido[1,2-a]pyrimidin-9-yl)ethyl)amino)benzoate

To a solution of 1-methyl-5-(4,4,5,5-tetramethyl-1,3,2-dioxaborolan-2-yl)indazol-3-amine (155 mg, 569 umol, prepared according to the procedure in WO2015140054), tert-butyl 2-[1-[7-methyl-4-oxo-2-(trifluoromethylsulfonyloxy)pyrido[1,2-a]pyrimidin-9-yl]ethylamino]benzoate (150 mg, 284 umol), K₂CO₃ (118 mg, 853 umol) in dioxane (2 mL) and water (0.2 mL) was added Pd(PPh₃)₄ (33 mg, 28 umol). The reaction mixture was stirred at 100° C. for 2 h under N₂ atmosphere. After cooling to room temperature, EtOAc (30 mL) was added, and the mixture was washed with brine (30 mL). The organic layer was dried over anhydrous Na₂SO₄, filtered and concentrated in vacuo. The crude residue was purified by silica gel chromatography (solvent gradient: 0-30% EtOAc in petroleum ether) to give the title compound (125 mg, 84%) as a brown solid. MS: m/z 525.1 (M+H⁺).

### Step 2: Synthesis of tert-butyl (R)-2-((1-(2-(3-amino-1-methyl-1H-indazol-5-yl)-7-methyl-4-oxo-4H-pyrido[1,2-a]pyrimidin-9-yl)ethyl)amino)benzoate & tert-butyl (S)-2-((1-(2-(3-amino-1-methyl-1H-indazol-5-yl)-7-methyl-4-oxo-4H-pyrido[1,2-a]pyrimidin-9-yl)ethyl)amino)benzoate

tert-butyl 2-((1-(2-(3-amino-1-methyl-1H-indazol-5-yl)-7-methyl-4-oxo-4H-pyrido[1,2-a]pyrimidin-9-yl)ethyl)amino)benzoate (125 mg, 238 umol) was separated by using chiral SFC (DAICEL CHIRALCEL OD-H (250 mm*30 mm, 5 um); Supercritical CO₂/EtOH+0.1% NH₃·H₂O=65/35; 60 ml/min) to afford tert-butyl (R)-2-((1-(2-(1,3-dimethyl-1H-indazol-5-yl)-7-methyl-4-oxo-4H-pyrido[1,2-a]pyrimidin-9-yl)ethyl)amino)benzoate (60 mg, first peak) and tert-butyl (S)-2-((1-(2-(1,3-dimethyl-1H-indazol-5-yl)-7-methyl-4-oxo-4H-pyrido[1,2-a]pyrimidin-9-yl)ethyl)amino)benzoate (60 mg, second peak) both as yellow solid. Absolute configuration was arbitrarily assigned to each enantiomer.

### Step 3: Synthesis of (S)-2-((1-(2-(3-amino-1-methyl-1H-indazol-5-yl)-7-methyl-4-oxo-4H-pyrido[1,2-a]pyrimidin-9-yl)ethyl)amino)benzoic acid (Compound 536)

To a mixture of tert-butyl (S)-2-((1-(2-(1,3-dimethyl-1H-indazol-5-yl)-7-methyl-4-oxo-4H-pyrido[1,2-a]pyrimidin-9-yl)ethyl)amino)benzoate (60 mg, 114 umol) in DCM (3 mL) was added TFA (1 mL). The reaction was stirred at room temperature for 16 h. This mixture was concentrated in vacuo and then diluted with MeOH (2 mL). The solution was adjusted pH to 10 with aq. LiOH (2 M), then adjusted pH to 6 with formic acid. The solution was purified by reverse phase chromatography (acetonitrile 3-33%/0.225% formic acid in water) to give the title compound (Compound 536; 18 mg, 33%) as a yellow solid. ¹H NMR (400 MHz, DMSO-d₆): δ 8.76 (s, 1H), 8.72 (s, 1H), 8.67-8.57 (m, 1H), 8.23-8.21 (m, 1H), 7.83-7.81 (m, 1H), 7.73-7.72 (m, 1H), 7.43 (d, J=8.8 Hz, 1H), 7.19-7.15 (m, 1H), 6.95 (s, 1H), 6.53 (t, J=7.6 Hz, 1H), 6.40 (d, J=8.4 Hz, 1H), 5.63 (s, 1H), 3.77 (s, 3H), 2.33 (s, 3H), 1.68 (d, J=6.4 Hz, 3H). MS: m/z 468.5 (M+H⁺).

### Example 9: Preparation of rel-(R)-2-((1-(2-(3-amino-1-methyl-1H-indazol-5-yl)-7-methyl-4-oxo-4H-pyrido[1,2-a]pyrimidin-9-yl)ethyl)amino)benzoic acid (Compound 535) and rel-(R)-2-((1-(2-(3-amino-1-methyl-1H-indazol-5-yl)-7-methyl-4-oxo-4H-pyrido[1,2-a]pyrimidin-9-yl)ethyl)amino)benzoic acid (Compound 536)

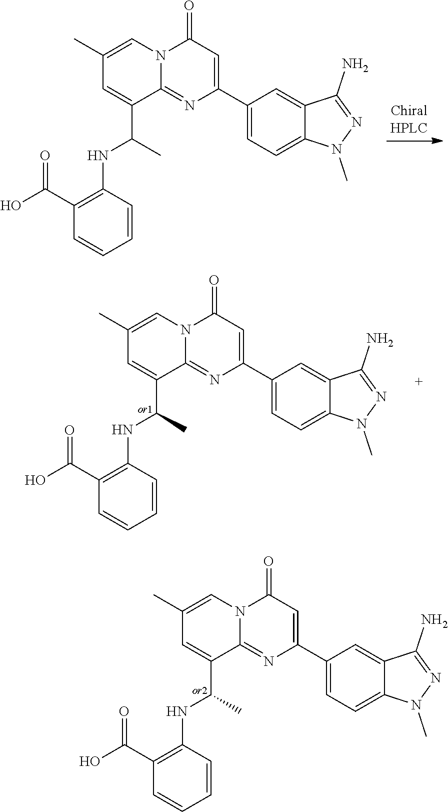 [SMILES: Cc1cc(C(C)Nc2ccccc2C(=O)O)c2nc(-c3ccc4c(c3)c(N)nn4C)cc(=O)n2c1 ; Cc1cc([C@@H](C)Nc2ccccc2C(=O)O)c2nc(-c3ccc4c(c3)c(N)nn4C)cc(=O)n2c1 ; Cc1cc([C@H](C)Nc2ccccc2C(=O)O)c2nc(-c3ccc4c(c3)c(N)nn4C)cc(=O)n2c1]

2-({1-[2-(3-amino-1-methylindazol-5-yl)-7-methyl-4-oxopyrido[1,2-a]pyrimidin-9-yl]ethyl}amino)benzoic acid (100 mg, 0.213 mmol, 1 equiv) was isolated by prep-chiral-HPLC with the following conditions: Column: CHIRAL ART Cellulose-SB, 2*25 cm, 5 m; Mobile Phase A: Hex (0.1% FA)-HPLC, Mobile Phase B: EtOH-HPLC; Flow rate: 20 mL/min; Gradient: 30% B to 30% B (isocratic) in 18 min; Wave Length: 220/203 nm; RT1 (min): 9.69; RT2 (min): 14.55; Sample Solvent: HFIP; Injection Volume: 0.5 mL; Number Of Runs: 8. The 1^{st} eluting fraction was concentrated under vacuum and lyophilized to afford rel-(R)-2-((1-(2-(3-amino-1-methyl-1H-indazol-5-yl)-7-methyl-4-oxo-4H-pyrido[1,2-a]pyrimidin-9-yl)ethyl)amino)benzoic acid (Compound 535; 26.3 mg, 25.98%) as an yellow solid. The 2^{nd} eluting fraction was concentrated under vacuum and lyophilized to afford rel-(R)-2-((1-(2-(3-amino-1-methyl-1H-indazol-5-yl)-7-methyl-4-oxo-4H-pyrido[1,2-a]pyrimidin-9-yl)ethyl)amino)benzoic acid (Compound 536; 13.4 mg, 13.28%) as a yellow solid.

Compound 535: ¹H NMR (400 MHz, DMSO-d₆) δ 8.98-8.72 (m, 2H), 8.71-8.33 (m, 1H), 8.23 (dd, J=8.9, 1.6 Hz, 1H), 7.84 (dd, J=8.0, 1.7 Hz, 1H), 7.73 (d, J=2.0 Hz, 1H), 7.44 (d, J=9.0 Hz, 1H), 7.20 (ddd, J=8.7, 7.1, 1.7 Hz, 1H), 6.97 (s, 1H), 6.55 (ddd, J=8.1, 7.1, 1.0 Hz, 1H), 6.43 (d, J=8.5 Hz, 1H), 5.66 (s, 1H), 3.78 (s, 3H), 2.33 (d, J=1.2 Hz, 3H), 1.70 (d, J=6.6 Hz, 3H). LC-MS: (ES+H, m/z): [M+H]⁺=469.2.

Compound 536: ¹H NMR (400 MHz, DMSO-d₆) δ 8.91-8.68 (m, 2H), 8.52 (s, 1H), 8.23 (dd, J=8.9, 1.7 Hz, 1H), 7.83 (dd, J=8.0, 1.8 Hz, 1H), 7.73 (d, J=2.1 Hz, 1H), 7.44 (d, J=8.9 Hz, 1H), 7.20 (ddd, J=8.7, 7.1, 1.8 Hz, 1H), 6.96 (s, 1H), 6.55 (t, J=7.5 Hz, 1H), 6.43 (d, J=8.5 Hz, 1H), 5.65 (s, 1H), 3.78 (s, 3H), 2.44-2.22 (m, 3H), 1.70 (d, J=6.5 Hz, 3H). LC-MS: (ES+H, m/z): [M+H]⁺=469.2.

### Example 10: Preparation of rel-2-{[(1R)-1-[7-methyl-4-oxo-2-(pyridin-4-yl)pyrido[1,2-a]pyrimidin-9-yl]ethyl]amino}benzamide (Compound 439) and rel-2-{[(1R)-1-[7-methyl-4-oxo-2-(pyridin-4-yl)pyrido[1,2-a]pyrimidin-9-yl]ethyl]amino}benzamide (Compound 440)

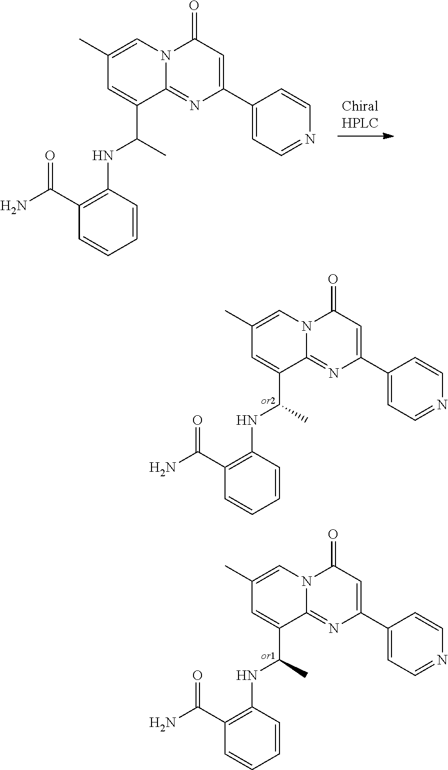 [SMILES: Cc1cc(C(C)Nc2ccccc2C(N)=O)c2nc(-c3ccncc3)cc(=O)n2c1 ; Cc1cc([C@H](C)Nc2ccccc2C(N)=O)c2nc(-c3ccncc3)cc(=O)n2c1 ; Cc1cc([C@@H](C)Nc2ccccc2C(N)=O)c2nc(-c3ccncc3)cc(=O)n2c1]

2-({1-[7-methyl-4-oxo-2-(pyridin-4-yl)pyrido[1,2-a]pyrimidin-9-yl]ethyl}amino)benzamide (76 mg, 0.190 mmol, 1 equiv) was isolated by Prep-CHIRAL-HPLC with the following conditions: Column: CHIRAL ART Cellulose-SZ, 3*25 cm, 5 m; Mobile Phase A: Hex (10 mM NH3-MeOH), Mobile Phase B: EtOH-HPLC; Flow rate: 20 mL/min; Gradient: 50% B to 50% B (isocratic) in 37 min; Wave Length: 206/256 nm; RT1 (min): 18.85; RT2 (min): 26.65; Sample Solvent: MeOH:DCM=1:2; Injection Volume: 0.7 mL; Number Of Runs: 5; The 1^{st} eluting fraction was concentrated under reduced pressure to afford rel-2-{[(1R)-1-[7-methyl-4-oxo-2-(pyridin-4-yl)pyrido[1,2-a]pyrimidin-9-yl]ethyl]amino}benzamide (Compound 439; 26.9 mg, 35.39%, ee=100%) as a white solid. The 2nd eluting fraction was concentrated under reduced pressure to afford rel-2-{[(1R)-1-[7-methyl-4-oxo-2-(pyridin-4-yl)pyrido[1,2-a]pyrimidin-9-yl]ethyl]amino}benzamide (Compound 440; 34.1 mg, 44.87%, ee=99.59%) as a white solid.

Compound 439: ¹H NMR (300 MHz, DMSO-d₆) δ 8.85 (d, J=6.4 Hz, 1H), 8.81-8.75 (m, 3H), 8.25-8.20 (m, 2H), 7.93 (s, 1H), 7.79 (d, J=2.0 Hz, 1H), 7.67-7.61 (m, 1H), 7.28 (s, 1H), 7.21 (s, 1H), 7.15-7.06 (m, 1H), 6.56-6.43 (m, 1H), 6.36 (d, J=8.4 Hz, 1H), 5.56-5.44 (m, 1H), 2.40-2.31 (m, 3H), 1.64 (d, J=6.6 Hz, 3H). LC-MS: (ES+H, nm z): [M+H]⁺=400.1

Compound 440: ¹H NMR (300 MHz, DMSO-d₆) δ 8.85 (d, J=6.3 Hz, 1H), 8.81-8.72 (m, 3H), 8.25-8.20 (m, 2H), 7.93 (s, 1H), 7.79 (d, J=2.0 Hz, 1H), 7.67-7.61 (m, 1H), 7.28 (s, 1H), 7.21 (d, J=2.0 Hz, 1H), 7.15-7.06 (m, 1H), 6.56-6.43 (m, 1H), 6.36 (d, J=8.4 Hz, 1H), 5.56-5.44 (m, 1H), 2.40-2.31 (m, 3H), 1.64 (d, J=6.5 Hz, 3H). LC-MS: (ES+H, m/z): [M+H]⁺=400.1

### Example 11: Preparation of 2-((1-(7-methyl-2-(7-methyl-2-oxo-2,3-dihydrobenzo[d]oxazol-5-yl)-4-oxo-4H-pyrido[1,2-a]pyrimidin-9-yl)ethyl)amino)benzoic acid (Compound 1413)

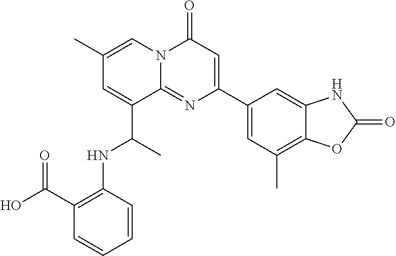 [SMILES: Cc1cc(C(C)Nc2ccccc2C(=O)O)c2nc(-c3cc(C)c4oc(=O)[nH]c4c3)cc(=O)n2c1]

### Step 1: Synthesis of 7-methyl-5-(4,4,5,5-tetramethyl-1,3,2-dioxaborolan-2-yl)benzo[d]oxazol-2(3H)-one

To a solution of 5-chloro-7-methyl-3H-1,3-benzoxazol-2-one (0.3 g, 1.63 mmol) (prepared according to the procedure in US2015210655) and 4,4,5,5-tetramethyl-2-(4,4,5,5-tetramethyl-1,3,2-dioxaborolan-2-yl)-1,3,2-dioxaborolane (830 mg, 3.27 mmol) in dioxane (10 mL) was added Xphos-Pd-G2 (128 mg, 163 umol) and KOAc (401 mg, 4.09 mmol). The reaction mixture was stirred at 110° C. for 2 hours under N₂ atmosphere. After cooling to room temperature, the reaction was filtered and concentrated in vacuo. The crude residue was purified by silica gel chromatography (solvent gradient: 0-30% EtOAc in petroleum ether) to give 7-methyl-5-(4,4,5,5-tetramethyl-1,3,2-dioxaborolan-2-yl)benzo[d]oxazol-2(3H)-one (200 mg, 45%) as a white solid. ¹H NMR (400 MHz, CDCl₃) δ 8.71 (s, 1H), 7.44 (s, 1H), 7.33 (s, 1H), 2.39 (s, 3H), 1.35 (s, 12H). MS: m/z 276.1 (M+H⁺).

### Step 2—Synthesis of tert-butyl 2-((1-(7-methyl-2-(7-methyl-2-oxo-2,3-dihydrobenzo[d]oxazol-5-yl)-4-oxo-4H-pyrido[1,2-a]pyrimidin-9-yl)ethyl)amino)benzoate

To a solution of tert-butyl 2-((1-(7-methyl-4-oxo-2-(((trifluoromethyl)sulfonyl)oxy)-4H-pyrido[1,2-a]pyrimidin-9-yl)ethyl)amino)benzoate (70 mg, 132 umol) and 7-methyl-5-(4,4,5,5-tetramethyl-1,3,2-dioxaborolan-2-yl)benzo[d]oxazol-2(3H)-one (73 mg, 265 umol) in dioxane (6 mL) and water (1.5 mL) was added Pd(PPh₃)₄ (15 mg, 13 umol) and K₂CO₃ (55 mg, 398 umol). The mixture was stirred at 100° C. for 4 h under N₂ atmosphere. After cooling to room temperature, the reaction was concentrated in vacuo. The crude residue was purified by silica gel chromatography (solvent gradient: 0-50% EtOAc in petroleum ether) to tert-butyl 2-((1-(7-methyl-2-(7-methyl-2-oxo-2,3-dihydrobenzo[d]oxazol-5-yl)-4-oxo-4H-pyrido[1,2-a]pyrimidin-9-yl)ethyl)amino)benzoate (40 mg, 58%) as a white solid. ¹H NMR (400 MHz, DMSO-d₆) δ 11.76 (s, 1H), 8.75 (s, 1H), 8.34 (d, J=4.8 Hz, 1H), 7.90 (s, 1H), 7.82-7.80 (m, 2H), 7.77 (d, J=8.0 Hz, 1H), 7.21 (t, J=7.2 Hz, 1H), 7.02 (s, 1H), 6.55 (t, J=7.6 Hz, 1H), 6.46 (d, J=8.4 Hz, 1H), 5.57-5.44 (m, 1H), 2.40 (s, 3H), 2.35 (s, 3H), 1.70 (d, J=6.4 Hz, 3H), 1.53 (s, 9H). MS: m/z 527.2 (M+H⁺).

### Step 3—Synthesis of 2-((1-(7-methyl-2-(7-methyl-2-oxo-2,3-dihydrobenzo[d]oxazol-5-yl)-4-oxo-4H-pyrido[1,2-a]pyrimidin-9-yl)ethyl)amino)benzoic acid (Compound 319)

To a mixture of tert-butyl 2-((1-(7-methyl-2-(7-methyl-2-oxo-2,3-dihydrobenzo[d]oxazol-5-yl)-4-oxo-4H-pyrido[1,2-a]pyrimidin-9-yl)ethyl)amino)benzoate (30 mg, 57 umol) in DCM (1.5 mL) was added TFA (0.5 mL). The reaction mixture was stirred at room temperature for 5 hours. The mixture was concentrated in vacuo. The residue was adjusted pH to 6 with NH₃·H₂O (25% w/w) solution. The resulting mixture was purified by Prep-TLC (10% MeOH in DCM) to give 2-((1-(7-methyl-2-(7-methyl-2-oxo-2,3-dihydrobenzo[d]oxazol-5-yl)-4-oxo-4H-pyrido[1,2-a]pyrimidin-9-yl)ethyl)amino)benzoic acid (7 mg, 23%) as a white solid. ¹H NMR (400 MHz, DMSO-d₆) δ 12.72 (s, 1H), 11.73 (s, 1H), 8.74 (s, 1H), 8.53 (s, 1H), 7.90 (s, 1H), 7.85-7.75 (m, 3H), 7.20 (t, J=8.0 Hz, 1H), 7.03 (s, 1H), 6.55 (t, J=7.6 Hz, 1H), 6.41 (d, J=8.4 Hz, 1H), 5.58-5.49 (m, 1H), 2.40 (s, 3H), 2.34 (s, 3H), 1.67 (d, J=6.4 Hz, 3H). MS: m/z 471.2 (M+H⁺).

### Example 12: Preparation of (S)-2-((1-(2-(isoindolin-2-yl)-7-methyl-4-oxo-4H-pyrido[1,2-a]pyrimidin-9-yl)ethyl)amino)benzoic acid (Compound 393)

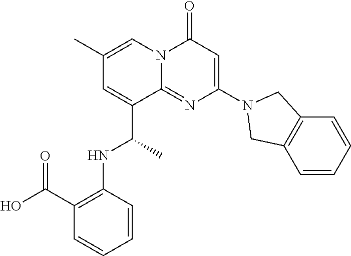 [SMILES: Cc1cc([C@H](C)Nc2ccccc2C(=O)O)c2nc(N3Cc4ccccc4C3)cc(=O)n2c1]

### Step 1—tert-butyl 2-((1-(2-(isoindolin-2-yl)-7-methyl-4-oxo-4H-pyrido[1,2-a]pyrimidin-9-yl)ethyl)amino)benzoate

To a solution of tert-butyl 2-((1-(2-hydroxy-7-methyl-4-oxo-4H-pyrido[1,2-a]pyrimidin-9-yl)ethyl)amino)benzoate (150 mg, 379 umol) in DMF (2 mL) was added DIEA (198 uL, 1.14 mmol) and PyBOP (236 mg, 455 umol). The reaction mixture was stirred at room temperature for 10 minutes, then isoindoline (135 mg, 1.14 mmol) was added. The reaction mixture was stirred at 40° C. for 16 hours. After cooling to room temperature, the reaction was quenched with water (10 mL) and extracted with EtOAc (50 mL×3). The combined organic layers were washed with aq. HCl (1 M, 30 mL) and brine (30 mL), dried over anhydrous Na₂SO₄, filtered and concentrated in vacuo. The crude residue was purified by silica gel chromatography (solvent gradient: 0-30% EtOAc in petroleum ether) to give tert-butyl 2-((1-(2-(isoindolin-2-yl)-7-methyl-4-oxo-4H-pyrido[1,2-a]pyrimidin-9-yl)ethyl)amino)benzoate (150 mg, 79%) as a white solid. MS: m/z 497.2 (M+H⁺).

### Step 2—Synthesis of 2-((1-(2-(isoindolin-2-yl)-7-methyl-4-oxo-4H-pyrido[1,2-a]pyrimidin-9-yl)ethyl)amino)benzoic acid

To a solution of tert-butyl 2-((1-(2-(isoindolin-2-yl)-7-methyl-4-oxo-4H-pyrido[1,2-a]pyrimidin-9-yl)ethyl)amino)benzoate (150 mg, 302 umol) in DCM (3 mL) was added TFA (1 mL). The reaction mixture was stirred at room temperature for 16 hours. The reaction mixture was concentrated in vacuo. The residue was purified by Prep-TLC (10% MeOH in DCM) to give 2-((1-(2-(isoindolin-2-yl)-7-methyl-4-oxo-4H-pyrido[1,2-a]pyrimidin-9-yl)ethyl)amino)benzoic acid (100 mg, 68%) as a yellow solid. MS: m/z 441.2 (M+H⁺).

### Step 3—(R)-2-((1-(2-(isoindolin-2-yl)-7-methyl-4-oxo-4H-pyrido[1,2-a]pyrimidin-9-yl)ethyl)amino)benzoic acid & (S)-2-((1-(2-(isoindolin-2-yl)-7-methyl-4-oxo-4H-pyrido[1,2-a]pyrimidin-9-yl)ethyl)amino)benzoic acid (Compound 403)

2-((1-(2-(isoindolin-2-yl)-7-methyl-4-oxo-4H-pyrido[1,2-a]pyrimidin-9-yl)ethyl)amino)benzoic acid (100 mg, 227 umol) was separated by using chiral SFC (DAICEL CHIRALCEL OJ-H (250 mm*30 mm, 5 um); Supercritical CO₂/EtOH+0.1% NH₃·H₂O=70/30; 60 ml/min) to afford (R)-2-((1-(2-(isoindolin-2-yl)-7-methyl-4-oxo-4H-pyrido[1,2-a]pyrimidin-9-yl)ethyl)amino)benzoic acid (41 mg, first peak) and (S)-2-((1-(2-(isoindolin-2-yl)-7-methyl-4-oxo-4H-pyrido[1,2-a]pyrimidin-9-yl)ethyl)amino)benzoic acid (34 mg, second peak) both as white solid. Absolute configuration was arbitrarily assigned to each enantiomer. Compound 403: ¹H NMR (400 MHz, DMSO-d₆) δ 8.58 (s, 2H), 7.80 (dd, J=8.0, 1.6 Hz, 1H), 7.64 (d, J=1.6 Hz, 1H), 7.44-7.40 (m, 2H), 7.35-7.29 (m, 2H), 7.23 (t, J=7.2 Hz, 1H), 6.53 (t, J=7.2 Hz, 1H), 6.47 (d, J=8.8 Hz, 1H), 5.42 (s, 1H), 5.38-5.31 (m, 1H), 5.11-4.86 (m, 2H), 4.78-4.68 (m, 2H), 2.25 (s, 3H), 1.66 (d, J=6.8 Hz, 3H). MS: m/z 441.1 (M+H⁺).

### Example 13: Preparation of (R)-2-((1-(2-(isoindolin-2-yl)-7-methyl-4-oxo-4H-pyrido[1,2-a]pyrimidin-9-yl)ethyl)amino)benzamide (Compound 620)

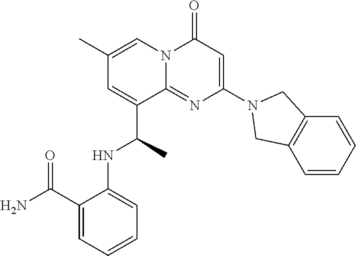 [SMILES: Cc1cc([C@@H](C)Nc2ccccc2C(N)=O)c2nc(N3Cc4ccccc4C3)cc(=O)n2c1]

### Step 1—Synthesis of 9-bromo-2-(isoindolin-2-yl)-7-methyl-4H-pyrido[1,2-a]pyrimidin-4-one

To a solution of 9-bromo-2-hydroxy-7-methyl-4H-pyrido[1,2-a]pyrimidin-4-one (5 g, 20 mmol) in DMF (100 mL) was added PyBOP (12 g, 23 mmol) and DIEA (10.2 mL, 59 mmol). The mixture was stirred at room temperature for 20 minutes, isoindoline hydrochloride (4.7 g, 30 mmol) was added.

The reaction was stirred at 40° C. for 16 hours. After cooling to room temperature, the reaction was quenched with water (100 mL), and extracted with EtOAc (200 mL×3). The combined organic layers were washed with aq. HCl (1 M, 200 mL), brine (200 mL×3), dried over anhydrous Na₂SO₄, filtered and concentrated in vacuo. The crude residue was purified by silica gel chromatography (solvent gradient: 0-20% EtOAc in petroleum ether) to give 9-bromo-2-(isoindolin-2-yl)-7-methyl-4H-pyrido[1,2-a]pyrimidin-4-one (6.5 g, 93%) as a white solid. ¹H NMR (400 MHz, DMSO-d₆) δ 8.67 (d, J=1.6 Hz, 1H), 8.22 (d, J=1.6 Hz, 1H), 7.49-7.41 (m, 2H), 7.36-7.32 (m, 2H), 5.42 (s, 1H), 4.99-4.92 (m, 2H), 4.78-4.69 (m, 2H), 2.25 (s, 3H). MS: m/z 358.0 (M+H⁺).

### Step 2—Synthesis of 9-(1-ethoxyvinyl)-2-(isoindolin-2-yl)-7-methyl-4H-pyrido[1,2-a]pyrimidin-4-one

A mixture of 9-bromo-2-(isoindolin-2-yl)-7-methyl-4H-pyrido[1,2-a]pyrimidin-4-one (6.5 g, 18 mmol), tributyl(1-ethoxyvinyl)stannane (14 g, 39 mmol), Pd(dppf)Cl₂ (668 mg, 912 umol) in dioxane (100 mL) was stirred at 100° C. for 16 h under N₂ atmosphere. After cooling to room temperature, the mixture was quenched with 100 mL of 10% KF aqueous solution, stirred for 30 min. The mixture was extracted with EtOAc (100 mL×2). The combined organic layers were washed with brine (100 mL), dried over Na₂SO₄, filtered and concentrated in vacuo. The crude residue was purified by silica gel chromatography (solvent gradient: 0-5% MeOH in DCM) to give 9-(1-ethoxyvinyl)-2-(isoindolin-2-yl)-7-methyl-4H-pyrido[1,2-a]pyrimidin-4-one (4.1 g, 64%) as a brown solid. ¹H NMR (400 MHz, DMSO-d₆) δ 8.71 (s, 1H), 7.94 (d, J=2.4 Hz, 1H), 7.45-7.41 (m, 2H), 7.37-7.30 (m, 2H), 5.65 (s, 1H), 5.42 (s, 1H), 4.89 (s, 2H), 4.80 (s, 1H), 4.72 (s, 2H), 3.96 (q, J=6.8 Hz, 2H), 2.34 (s, 3H), 1.37 (t, J=6.8 Hz, 3H). MS: m/z 348.2 (M+H⁺).

### Step 3—Synthesis of 9-acetyl-2-(isoindolin-2-yl)-7-methyl-4H-pyrido[1,2-a]pyrimidin-4-one

To a solution of 9-(1-ethoxyvinyl)-2-(isoindolin-2-yl)-7-methyl-4H-pyrido[1,2-a]pyrimidin-4-one (1 g, 2.88 mmol) in THF (10 mL) was added 1M HCl (5 mL). The reaction mixture was stirred at room temperature for 3 hours. The mixture was adjusted to pH 7 with sat. aq. NaHCO₃, then extracted with EtOAc (60 mL×2). The combined organic layers were washed with brine (50 mL), dried over Na₂SO₄, filtered and concentrated in vacuo to give 9-acetyl-2-(isoindolin-2-yl)-7-methyl-4H-pyrido[1,2-a]pyrimidin-4-one (0.9 g, crude) as a brown solid that required no further purification. ¹H NMR (400 MHz, DMSO-d₆) δ 8.82 (s, 1H), 7.94 (d, J=2.0 Hz, 1H), 7.49-7.38 (m, 2H), 7.37-7.30 (m, 2H), 5.43 (s, 1H), 4.93 (s, 2H), 4.73 (s, 2H), 2.84 (s, 3H), 2.35 (s, 3H).

### Step 4—Synthesis of 9-(1-hydroxyethyl)-2-(isoindolin-2-yl)-7-methyl-4H-pyrido[1,2-a]pyrimidin-4-one

To a solution of 9-acetyl-2-(isoindolin-2-yl)-7-methyl-4H-pyrido[1,2-a]pyrimidin-4-one (1 g, 3.13 mmol) in MeOH (20 mL) was added NaBH₄ (142 mg, 3.76 mmol) at 0° C. The mixture was stirred at room temperature for 16 hours under N₂ atmosphere. The mixture was quenched with 1M HCl (10 mL) at 0° C., diluted with water (20 mL), extracted with EtOAc (30 mL×2). The combined organic layers were washed with brine (30 mL), dried over anhydrous Na₂SO₄, filtered and concentrated in vacuo. The crude residue was purified by silica gel chromatography (solvent gradient: 0-50% EtOAc in petroleum ether) to give 9-(1-hydroxyethyl)-2-(isoindolin-2-yl)-7-methyl-4H-pyrido[1,2-a]pyrimidin-4-one (0.97 g, 96%) as a yellow solid. ¹H NMR (400 MHz, DMSO-d₆) δ 8.57 (s, 1H), 7.78 (d, J=2.0 Hz, 1H), 7.42 (s, 2H), 7.35-7.29 (m, 2H), 5.42-5.27 (m, 3H), 4.99-4.65 (m, 4H), 2.33 (s, 3H), 1.47 (d, J=6.4 Hz, 3H). MS: m/z 322.2 (M+H⁺).

### Step 5—Synthesis of 9-(1-bromoethyl)-2-(isoindolin-2-yl)-7-methyl-4H-pyrido[1,2-a]pyrimidin-4-one

To a solution of 9-(1-hydroxyethyl)-2-(isoindolin-2-yl)-7-methyl-4H-pyrido[1,2-a]pyrimidin-4-one (957 mg, 2.98 mmol) in DCM (10 mL) was added PBr₃ (1.6 g, 5.91 mmol) at 0° C. The mixture was stirred at room temperature for 16 hours under N₂ atmosphere. The mixture was quenched with sat. aq. NaHCO₃ (30 mL), extracted with EtOAc (50 mL×2). The combined organic layers were washed with brine (30 mL), dried over anhydrous Na₂SO₄, filtered and concentrated in vacuo. The crude residue was purified by silica gel chromatography (solvent gradient: 0-50% EtOAc in petroleum ether) to give 9-(1-bromoethyl)-2-(isoindolin-2-yl)-7-methyl-4H-pyrido[1,2-a]pyrimidin-4-one (455 mg, 40%) as a yellow solid. ¹H NMR (400 MHz, CDCl₃) δ 8.77 (s, 1H), 7.76 (d, J=2.0 Hz, 1H), 7.41-7.32 (m, 4H), 6.16-6.09 (m, 1H), 5.58 (s, 1H), 5.19-4.95 (m, 2H), 4.75-4.68 (m, 2H), 2.39 (s, 3H), 2.14 (d, J=6.8 Hz, 3H).

### Step 6—Synthesis of 2-((1-(2-(isoindolin-2-yl)-7-methyl-4-oxo-4H-pyrido[1,2-a]pyrimidin-9-yl)ethyl)amino)benzamide

A mixture of 9-(1-bromoethyl)-2-(isoindolin-2-yl)-7-methyl-4H-pyrido[1,2-a]pyrimidin-4-one (100 mg, 260 umol) and 2-aminobenzamide (71 mg, 520 umol) in dioxane (1 mL) was stirred at 100° C. for 2 hours. After cooling to room temperature, the mixture was concentrated in vacuo. The crude residue was purified by reverse phase chromatography (acetonitrile 55%-85%/0.225% formic acid in water) to give 2-((1-(2-(isoindolin-2-yl)-7-methyl-4-oxo-4H-pyrido[1,2-a]pyrimidin-9-yl)ethyl)amino)benzamide (30 mg, 26%) as a white solid. ¹H NMR (400 MHz, DMSO-d₆) δ 8.80 (d, J=6.8 Hz, 1H), 8.58 (s, 1H), 7.88 (s, 1H), 7.64-7.57 (m, 2H), 7.45-7.41 (m, 2H), 7.36-7.30 (m, 2H), 7.23 (s, 1H), 7.16-7.12 (m, 1H), 6.53-6.49 (m, 1H), 6.39 (d, J=8.4 Hz, 1H), 5.43 (s, 1H), 5.36-5.25 (m, 1H), 5.12-4.87 (m, 2H), 4.76-4.70 (m, 2H), 2.25 (s, 3H), 1.61 (d, J=6.8 Hz, 3H). MS: m/z 440.2 (M+H⁺).

### Step 7—Synthesis of (R)-2-((1-(2-(isoindolin-2-yl)-7-methyl-4-oxo-4H-pyrido[1,2-a]pyrimidin-9-yl)ethyl)amino)benzamide (Compound 37) & (S)-2-((1-(2-(isoindolin-2-yl)-7-methyl-4-oxo-4H-pyrido[1,2-a]pyrimidin-9-yl)ethyl)amino)benzamide

2-((1-(2-(isoindolin-2-yl)-7-methyl-4-oxo-4H-pyrido[1,2-a]pyrimidin-9-yl)ethyl)amino)benzamide (20 mg, 45 umol) was separated by using chiral SFC (DAICEL CHIRALCEL AD (250 mm*30 mm, 10 um); Supercritical CO₂/EtOH+0.1% NH₃·H₂O=55/45; 60 mL/min) to afford (R)-2-((1-(2-(isoindolin-2-yl)-7-methyl-4-oxo-4H-pyrido[1,2-a]pyrimidin-9-yl)ethyl)amino)benzamide (7 mg, first peak) and (S)-2-((1-(2-(isoindolin-2-yl)-7-methyl-4-oxo-4H-pyrido[1,2-a]pyrimidin-9-yl)ethyl)amino)benzamide (5 mg, second peak) both as white solid. Absolute configuration was arbitrarily assigned to each enantiomer. Compound 646: ¹H NMR (400 MHz, DMSO-d₆) δ 8.79 (d, J=6.4 Hz, 1H), 8.58 (s, 1H), 7.89 (s, 1H), 7.63-7.58 (m, 2H), 7.46-7.40 (m, 2H), 7.36-7.30 (m, 2H), 7.23 (s, 1H), 7.15-7.11 (m, 1H), 6.52-6.48 (m, 1H), 6.39 (d, J=8.4 Hz, 1H), 5.43 (s, 1H), 5.33-5.27 (m, 1H), 5.09-4.88 (m, 2H), 4.78-4.68 (m, 2H), 2.25 (s, 3H), 1.61 (d, J=6.4 Hz, 3H). MS: m/z 440.1 (M+H⁺).

### Example 14: Preparation of 2-((1-(7-methyl-4-oxo-2-(pyridin-3-yl)-4H-pyrido[1,2-a]pyrimidin-9-yl)ethyl)amino)benzoic acid trifluoroacetate (Compound 416)

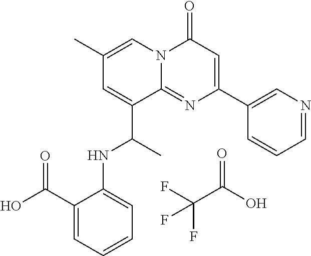

### Step 1—Synthesis of tert-butyl 2-((1-(7-methyl-4-oxo-2-(pyridin-3-yl)-4H-pyrido[1,2-a]pyrimidin-9-yl)ethyl)amino)benzoate

A mixture of tert-butyl 2-((1-(7-methyl-4-oxo-2-(((trifluoromethyl)sulfonyl)oxy)-4H-pyrido[1,2-a]pyrimidin-9-yl)ethyl)amino)benzoate (150 mg, 284 umol), 3-pyridylboronic acid (87 mg, 711 umol), Pd(PPh₃)₄ (66 mg, 57 umol), K₂CO₃ (118 mg, 853 umol) in dioxane (1.5 mL) and water (0.15 mL) was stirred at 100° C. for 2 hours under N₂ atmosphere. After cooling to room temperature, the reaction mixture was filtered and concentrated in vacuo. The crude residue was purified by silica gel chromatography (solvent gradient: 0-30% EtOAc in petroleum ether) to give tert-butyl 2-((1-(7-methyl-4-oxo-2-(pyridin-3-yl)-4H-pyrido[1,2-a]pyrimidin-9-yl)ethyl)amino)benzoate (100 mg, 77%) as a yellow solid. ¹H NMR (400 MHz, DMSO-d₆) δ 9.45 (s, 1H), 8.79 (s, 1H), 8.72 (d, J=4.0 Hz, 1H), 8.63 (d, J=8.0 Hz, 1H), 8.36 (d, J=6.0 Hz, 1H), 7.85 (s, 1H), 7.74-7.79 (m, 1H), 7.22-7.18 (m, 1H), 7.16 (s, 1H), 6.57-7.53 (m, 1H), 6.49 (d, J=8.4 Hz, 1H), 5.55-5.49 (m, 1H), 2.37 (s, 3H), 1.71 (d, J=6.8 Hz, 3H), 1.53 (s, 9H). MS: m/z 457.1 (M+H⁺).

### Step 2—Synthesis of 2-((1-(7-methyl-4-oxo-2-(pyridin-3-yl)-4H-pyrido[1,2-a]pyrimidin-9-yl)ethyl)amino)benzoic acid trifluoroacetate

To a solution of tert-butyl 2-((1-(7-methyl-4-oxo-2-(pyridin-3-yl)-4H-pyrido[1,2-a]pyrimidin-9-yl)ethyl)amino)benzoate (100 mg, 219 umol) in DCM (1.5 mL) was added TFA (0.5 mL). The mixture was stirred at 40° C. for 16 hours. After cooling to room temperature, the reaction mixture was concentrated in vacuo. The resulting residue was purified by reverse phase chromatography (acetonitrile 27%-57%/0.225% formic acid in water) to give 2-((1-(7-methyl-4-oxo-2-(pyridin-3-yl)-4H-pyrido[1,2-a]pyrimidin-9-yl)ethyl)amino)benzoic acid trifluoroacetate (Compound 426) (70 mg, 80%) as a white solid. ¹H NMR (400 MHz, DMSO-d₆) δ 12.75 (s, 1H), 9.45 (d, J=1.6 Hz, 1H), 8.78 (s, 1H), 8.72 (d, J=4.0 Hz, 1H), 8.63 (d, J=8.0 Hz, 1H), 8.51 (d, J=6.4 Hz, 1H), 7.79-7.83 (m, 2H), 7.58 (dd, J=8.0, 4.8 Hz, 1H), 7.17-7.23 (m, 1H), 7.17 (s, 1H), 6.57-6.53 (m, 1H), 6.44 (d, J=8.4 Hz, 1H), 5.58-5.52 (m, 1H), 2.36 (s, 3H), 1.68 (d, J=6.4 Hz, 3H). MS: m/z 401.0 (M+H⁺).

### Example 15: Preparation of 2-((1-(2-(isoindolin-2-yl)-3,7-dimethyl-4-oxo-4H-pyrido[1,2-a]pyrimidin-9-yl)ethyl)amino)benzoic acid trifluoroacetate (Compound 1412)

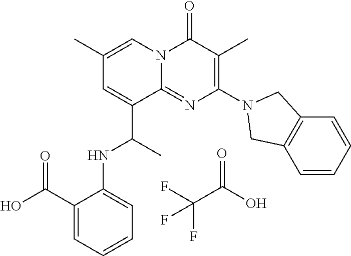

### Step 1—Synthesis of tert-butyl 2-((1-(2-(isoindolin-2-yl)-3,7-dimethyl-4-oxo-4H-pyrido[1,2-a]pyrimidin-9-yl)ethyl)amino)benzoate

To a solution of tert-butyl 2-((1-(2-hydroxy-3,7-dimethyl-4-oxo-4H-pyrido[1,2-a]pyrimidin-9-yl)ethyl)amino)benzoate (150 mg, 366 umol) in DMF (3 mL) was added DIEA (447 uL, 2.56 mmol) and PyBOP (248 mg, 476 umol). The mixture was stirred at room temperature for 20 min, isoindoline hydrochloride (257 mg, 1.65 mmol) was added. The final mixture was stirred at 50° C. for 16 hours. After cooling to room temperature, the reaction mixture was poured into 1M aq. HCl (30 mL). The suspension solution was filtered, the filter cake was dissolved in EtOAc (100 mL). The mixture was washed with brine (30 mL), dried over anhydrous Na₂SO₄, concentrated in vacuo. The crude residue was purified by silica gel chromatography (solvent gradient: 0-30% EtOAc in petroleum ether) to give tert-butyl 2-((1-(2-(isoindolin-2-yl)-3,7-dimethyl-4-oxo-4H-pyrido[1,2-a]pyrimidin-9-yl)ethyl)amino)benzoate (110 mg, 32%) as a white solid. MS: m/z 511.2 (M+H⁺).

### Step 2—Synthesis of 2-((1-(2-(isoindolin-2-yl)-3,7-dimethyl-4-oxo-4H-pyrido[1,2-a]pyrimidin-9-yl)ethyl)amino)benzoic acid trifluoroacetate (Compound 764)

To a solution of tert-butyl 2-((1-(2-(isoindolin-2-yl)-3,7-dimethyl-4-oxo-4H-pyrido[1,2-a]pyrimidin-9-yl)ethyl)amino)benzoate (30 mg, 59 umol) in DCM (1.5 mL) was added TFA (0.5 ml). The mixture was stirred at 40° C. for 16 hours. After cooling to room temperature, the reaction mixture was concentrated in vacuo. The resulting residue was purified by reverse phase chromatography (acetonitrile 54%-84%/0.225% formic acid in water) to give 2-((1-(2-(isoindolin-2-yl)-3,7-dimethyl-4-oxo-4H-pyrido[1,2-a]pyrimidin-9-yl)ethyl)amino)benzoic acid trifluoroacetate (Compound 764) (11 mg, 41%) as a yellow solid. ¹H NMR (400 MHz, DMSO-d₆) δ 12.73 (s, 1H), 8.56 (s, 1H), 8.43 (d, J=5.6 Hz, 1H), 7.80 (d, J=8.0 Hz, 1H), 7.53-7.59 (m, 1H), 7.36-7.43 (m, 2H), 7.27-7.34 (m, 2H), 7.26-7.20 (m, 1H), 6.56-6.52 (m, 1H), 6.41 (d, J=8.0 Hz, 1H), 5.38-5.32 (m, 1H), 5.23-5.13 (m, 4H), 2.42 (s, 3H), 2.26 (s, 3H), 1.64 (d, J=6.4 Hz, 3H). MS: m/z 455.0 (M+H⁺).

### Example 16: Preparation of (R)-2-((1-(2-(4,4-dimethyl-1,4-azasilinan-1-yl)-7-methyl-4-oxo-4H-pyrido[1,2-a]pyrimidin-9-yl)ethyl)amino)benzoic acid (Compound 431)

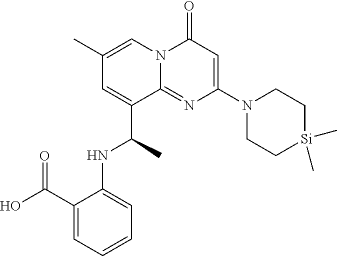 [SMILES: Cc1cc([C@@H](C)Nc2ccccc2C(=O)O)c2nc(N3CC[Si](C)(C)CC3)cc(=O)n2c1]

### Step 1—Synthesis of tert-butyl 2-((1-(2-(4,4-dimethyl-1,4-azasilinan-1-yl)-7-methyl-4-oxo-4H-pyrido[1,2-a]pyrimidin-9-yl)ethyl)amino)benzoate

To a solution of tert-butyl 2-((1-(2-hydroxy-7-methyl-4-oxo-4H-pyrido[1,2-a]pyrimidin-9-yl)ethyl)amino)benzoate (200 mg, 506 umol) in DMF (4 mL) was added DIEA (440 uL, 2.53 mmol) and PyBOP (316 mg, 607 umol). The mixture was stirred at room temperature for 30 minutes, 4,4-dimethyl-1,4-azasilinane hydrochloride (252 mg, 1.52 mmol) was added. The final mixture was stirred at 40° C. for 16 hours. After cooling to room temperature, the reaction mixture was quenched with water (30 mL), extracted with EtOAc (50 mL×2). The combined organic layers were washed with water (30 mL×3) and brine (30 mL), dried over anhydrous Na₂SO₄, concentrated in vacuo. The crude residue was purified by silica gel chromatography (solvent gradient: 0-50% EtOAc in petroleum ether) to give tert-butyl 2-((1-(2-(4,4-dimethyl-1,4-azasilinan-1-yl)-7-methyl-4-oxo-4H-pyrido[1,2-a]pyrimidin-9-yl)ethyl)amino)benzoate (200 mg, 78%) as a white solid. MS: m/z 507.3 (M+H⁺).

### Step 2—Synthesis of 2-((1-(2-(4,4-dimethyl-1,4-azasilinan-1-yl)-7-methyl-4-oxo-4H-pyrido[1,2-a]pyrimidin-9-yl)ethyl)amino)benzoic acid

To a solution of tert-butyl 2-((1-(2-(4,4-dimethyl-1,4-azasilinan-1-yl)-7-methyl-4-oxo-4H-pyrido[1,2-a]pyrimidin-9-yl)ethyl)amino)benzoate (150 mg, 296 umol) in DCM (6 mL) was added TFA (2 mL). The mixture was stirred at 50° C. for 16 hours. After cooling to room temperature, the reaction mixture was concentrated in vacuo. The crude residue was purified by reverse phase chromatography (acetonitrile 3%-33%/0.225% formic acid in water) to give 2-((1-(2-(4,4-dimethyl-1,4-azasilinan-1-yl)-7-methyl-4-oxo-4H-pyrido[1,2-a]pyrimidin-9-yl)ethyl)amino)benzoic acid (90 mg, 67%) as a white solid. ¹H NMR (400 MHz, DMSO-d₆) δ 12.74 (s, 1H), 8.52 (s, 1H), 8.40 (d, J=5.6 Hz, 1H), 7.80 (dd, J=8.0, 1.6 Hz, 1H), 7.56 (d, J=2.0 Hz, 1H), 7.25-7.17 (m, 1H), 6.56-6.52 (m, 1H), 6.35 (d, J=8.8 Hz, 1H), 5.59 (s, 1H), 5.26-5.19 (m, 1H), 3.89-3.83 (m, 4H), 2.23 (s, 3H), 1.58 (d, J=6.8 Hz, 3H), 0.86-0.74 (m, 4H), 0.10 (s, 6H). MS: m/z 451.2 (M+H⁺).

### Step 3—Synthesis of (R)-2-((1-(2-(4,4-dimethyl-1,4-azasilinan-1-yl)-7-methyl-4-oxo-4H-pyrido[1,2-a]pyrimidin-9-yl)ethyl)amino)benzoic acid (Compound 446) and (S)-2-((1-(2-(4,4-dimethyl-1,4-azasilinan-1-yl)-7-methyl-4-oxo-4H-pyrido[1,2-a]pyrimidin-9-yl)ethyl)amino)benzoic acid

2-((1-(2-(4,4-dimethyl-1,4-azasilinan-1-yl)-7-methyl-4-oxo-4H-pyrido[1,2-a]pyrimidin-9-yl)ethyl)amino)benzoic acid (85 mg, 189 umol) was separated by using chiral SFC (REGIS (S, S) WHELK-O1 (250 mm*25 mm, 10 um); Supercritical CO₂/EtOH+0.1% NH₃·H₂O=70/30; 80 mL/min) to afford (R)-2-((1-(2-(4,4-dimethyl-1,4-azasilinan-1-yl)-7-methyl-4-oxo-4H-pyrido[1,2-a]pyrimidin-9-yl)ethyl)amino)benzoic acid (16 mg, first peak) and (S)-2-((1-(2-(4,4-dimethyl-1,4-azasilinan-1-yl)-7-methyl-4-oxo-4H-pyrido[1,2-a]pyrimidin-9-yl)ethyl)amino)benzoic acid (25 mg, second peak) both as white solid. Absolute configuration was arbitrarily assigned to each enantiomer. Compound 446: ¹H NMR (400 MHz, DMSO-d₆) δ 8.53-8.43 (m, 2H), 7.81 (dd, J=8.0, 4.0 Hz, 1H), 7.56 (d, J=1.6 Hz, 1H), 7.20-7.16 (m, 1H), 6.55-6.51 (m, 1H), 6.33 (d, J=8.4 Hz, 1H), 5.59 (s, 1H), 5.27-5.17 (m, 1H), 3.90-3.84 (m, 4H), 2.22 (s, 3H), 1.57 (d, J=6.8 Hz, 3H), 0.84-0.73 (m, 4H), 0.10 (s, 6H). MS: m/z 451.2 (M+H⁺).

### Example 17: Preparation of 2-(((R)-1-(7-methyl-2-((S)-1-methylisoindolin-2-yl)-4-oxo-4H-pyrido[1,2-a]pyrimidin-9-yl)ethyl)amino)benzoic acid (Compound 652)

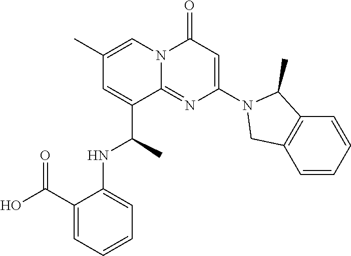 [SMILES: Cc1cc([C@@H](C)Nc2ccccc2C(=O)O)c2nc(N3Cc4ccccc4[C@@H]3C)cc(=O)n2c1]

### Step 1—Synthesis of tert-butyl 2-((1-(7-methyl-2-(1-methylisoindolin-2-yl)-4-oxo-4H-pyrido[1,2-a]pyrimidin-9-yl)ethyl)amino)benzoate

To a solution of tert-butyl 2-((1-(2-hydroxy-7-methyl-4-oxo-4H-pyrido[1,2-a]pyrimidin-9-yl)ethyl)amino)benzoate (200 mg, 506 umol) in DMF (2 mL) was added PyBOP (316 mg, 607 umol) and DIEA (440 uL, 2.53 mmol). The reaction mixture was stirred at room temperature for 10 min, then 1-methylisoindoline hydrochloride (129 mg, 759 umol) was added. The reaction mixture was stirred at 60° C. for 16 hours. After cooling to room temperature, the reaction was quenched with water (20 mL) and extracted with EtOAc (30 mL×3). The combined organic layers were washed with aq. HCl (1 M, 30 mL) and brine (30 mL), dried over anhydrous Na₂SO₄, filtered and concentrated in vacuo. The crude residue was purified by silica gel chromatography (solvent gradient: 0-70% EtOAc in petroleum ether) to give tert-butyl 2-((1-(7-methyl-2-(1-methylisoindolin-2-yl)-4-oxo-4H-pyrido[1,2-a]pyrimidin-9-yl)ethyl)amino)benzoate (200 mg, 77%) as a yellow solid. ¹H NMR (400 MHz, DMSO-d₆) δ 8.59 (d, J=0.8 Hz, 1H), 8.29 (d, J=5.2 Hz, 1H), 7.80-7.73 (m, 1H), 7.67-7.62 (m, 1H), 7.42-7.37 (m, 2H), 7.34-7.31 (m, 2H), 7.27-7.20 (m, 1H), 6.60-6.51 (m, 1H), 6.50-6.29 (m, 1H), 5.71-5.40 (m, 2H), 5.39-5.29 (m, 1H), 4.95-4.57 (m, 2H), 2.26 (s, 3H), 1.67 (d, J=6.8 Hz, 3H), 1.62 (s, 3H), 1.57-1.52 (m, 9H). MS: m/z 511.2 (M+H⁺).

### Step 2—Synthesis of tert-butyl 2-(((R)-1-(7-methyl-2-((S)-1-methylisoindolin-2-yl)-4-oxo-4H-pyrido[1,2-a]pyrimidin-9-yl)ethyl)amino)benzoate, tert-butyl 2-(((S)-1-(7-methyl-2-((S)-1-methylisoindolin-2-yl)-4-oxo-4H-pyrido[1,2-a]pyrimidin-9-yl)ethyl)amino)benzoate, tert-butyl 2-(((R)-1-(7-methyl-2-((R)-1-methylisoindolin-2-yl)-4-oxo-4H-pyrido[1,2-a]pyrimidin-9-yl)ethyl)amino)benzoate, tert-butyl 2-(((S)-1-(7-methyl-2-((R)-1-methylisoindolin-2-yl)-4-oxo-4H-pyrido[1,2-a]pyrimidin-9-yl)ethyl)amino)benzoate

tert-butyl 2-((1-(7-methyl-2-(1-methylisoindolin-2-yl)-4-oxo-4H-pyrido[1,2-a]pyriimidin-9-yl)ethyl)amino)benzoate (200 mg, 392 umol) was separated by using chiral SFC (DAICEL CHIRALCEL OD-H (250 mm*30 mm, 5 um); Supercritical CO₂/EtOH+0.1% NH₃·H₂O=75/25; 80 mL/min) to afford tert-butyl 2-(((R)-1-(7-methyl-2-((S)-1-methylisoindolin-2-yl)-4-oxo-4H-pyrido[1,2-a]pyrimidin-9-yl)ethyl)amino)benzoate (35 mg, first peak), tert-butyl 2-(((S)-1-(7-methyl-2-((S)-1-methylisoindolin-2-yl)-4-oxo-4H-pyrido[1,2-a]pyrimidin-9-yl)ethyl)amino)benzoate (33 mg, second peak), tert-butyl 2-(((R)-1-(7-methyl-2-((R)-1-methylisoindolin-2-yl)-4-oxo-4H-pyrido[1,2-a]pyrimidin-9-yl)ethyl)amino)benzoate (32 mg, third peak) and tert-butyl 2-(((S)-1-(7-methyl-2-((R)-1-methylisoindolin-2-yl)-4-oxo-4H-pyrido[1,2-a]pyrimidin-9-yl)ethyl)amino)benzoate (33 mg, fourth peak) both as yellow solid. Absolute configuration was arbitrarily assigned to each enantiomer. MS: m/z 511.2 (M+H⁺).

### Step 3—Synthesis of 2-(((R)-1-(7-methyl-2-((S)-1-methylisoindolin-2-yl)-4-oxo-4H-pyrido[1,2-a]pyrimidin-9-yl)ethyl)amino)benzoic acid

To a mixture of tert-butyl 2-(((R)-1-(7-methyl-2-((S)-1-methylisoindolin-2-yl)-4-oxo-4H-pyrido[1,2-a]pyrimidin-9-yl)ethyl)amino)benzoate (35 mg, 69 umol) in DCM (3 mL) was added TFA (1 mL). The reaction was stirred at room temperature for 16 hours. This mixture was concentrated in vacuo and then diluted with MeOH (2 mL). The solution was adjusted pH to 10 with aq. LiOH (2 M), then adjusted pH to 6 with formic acid. The solution was purified by reverse phase chromatography (acetonitrile 65-95%/0.225% formic acid in water) to give 2-(((R)-1-(7-methyl-2-((S)-1-methylisoindolin-2-yl)-4-oxo-4H-pyrido[1,2-a]pyrimidin-9-yl)ethyl)amino)benzoic acid (Compound 680) (13 mg, 40%) as a white solid. ¹H NMR (400 MHz, DMSO-d₆) δ 12.63 (s, 1H), 8.59 (s, 1H), 8.49 (s, 1H), 7.81 (d, J=6.8 Hz, 1H), 7.63 (s, 1H), 7.43-7.37 (m, 2H), 7.36-7.30 (m, 2H), 7.23-7.19 (m, 1H), 6.56-6.52 (m, 1H), 6.40 (s, 1H), 5.74-5.30 (m, 3H), 5.02-4.56 (m, 2H), 2.26 (s, 3H), 1.65-1.51 (m, 6H). MS: m/z 455.2 (M+H⁺).

### Example 18: Preparation of (R)-2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoic acid (Compound 593)

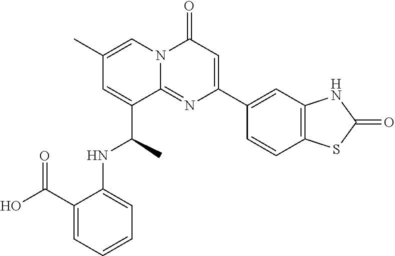 [SMILES: Cc1cc([C@@H](C)Nc2ccccc2C(=O)O)c2nc(-c3ccc4sc(=O)[nH]c4c3)cc(=O)n2c1]

### Step 1—Synthesis of 5-(4,4,5,5-tetramethyl-1,3,2-dioxaborolan-2-yl)benzo[d]thiazol-2(3H)-one

To a solution of 5-chloro-3H-1,3-benzothiazol-2-one (1 g, 5.39 mmol) and 4,4,5,5-tetramethyl-2-(4,4,5,5-tetramethyl-1,3,2-dioxaborolan-2-yl)-1,3,2-dioxaborolane (4.1 g, 16.16 mmol) in dioxane (10 mL) was added Xphos-Pd-G2 (424 mg, 539 umol) and KOAc (1.59 g, 16.16 mmol). The reaction mixture was stirred at 110° C. for 2 h under N₂ atmosphere. After cooling to room temperature, the reaction was filtered and concentrated in vacuo. The crude residue was purified by silica gel chromatography (solvent gradient: 0-20% EtOAc in petroleum ether) to give 5-(4,4,5,5-tetramethyl-1,3,2-dioxaborolan-2-yl)benzo[d]thiazol-2(3H)-one (1.4 g, 93%) as a yellow solid. ¹H NMR (400 MHz, DMSO-d₆) δ 11.94 (s, 1H), 7.58 (d, J=7.6 Hz, 1H), 7.39 (dd, J=7.6, 1.2 Hz, 1H), 7.36 (d, J=1.2 Hz, 1H), 1.29 (s, 12H). MS: m/z 277.8 (M+H⁺).

### Step 2—Synthesis of tert-butyl 2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoate

To a mixture of tert-butyl 2-((1-(7-methyl-4-oxo-2-(((trifluoromethyl)sulfonyl)oxy)-4H-pyrido[1,2-a]pyrimidin-9-yl)ethyl)amino)benzoate (150 mg, 284 umol) and 5-(4,4,5,5-tetramethyl-1,3,2-dioxaborolan-2-yl)benzo[d]thiazol-2(3H)-one (158 mg, 569 umol) in dioxane (2 mL) and water (0.2 mL) was added K₂CO₃ (118 mg, 853 umol) and Pd(PPh₃)₄ (33 mg, 29 umol).

The mixture was stirred at 100° C. for 2 hours under N₂ atmosphere. After cooling to room temperature, the reaction was concentrated in vacuo. The crude residue was purified by silica gel chromatography (solvent gradient: 0-40% EtOAc in petroleum ether) to give tert-butyl 2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoate (36 mg, 24%) as a white solid. ¹H NMR (400 MHz, DMSO-d₆) δ 12.06 (s, 1H), 8.76 (s, 1H), 8.34 (d, J=6.4 Hz, 1H), 8.06 (d, J=1.2 Hz, 1H), 8.01 (dd, J=8.4, 1.6 Hz, 1H), 7.81 (s, 1H), 7.79-7.71 (m, 2H), 7.22-7.18 (m, 1H), 7.01 (s, 1H), 6.59-6.52 (m, 2H), 6.45 (d, J=8.4 Hz, 1H), 5.56-5.50 (m, 1H), 2.36 (s, 3H), 1.71 (d, J=6.4 Hz, 3H), 1.53 (s, 9H). MS: m/z 529.2 (M+H⁺).

### Step 3—Synthesis of tert-butyl (R)-2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoate & tert-butyl (S)-2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoate

tert-butyl 2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoate (94 mg, 178 umol) was separated by using chiral SFC (DAICEL CHIRALCEL OD-H (250 mm*30 mm, 5 um); Supercritical CO₂/EtOH+0.1% NH₃·H₂O=50/50; 60 ml/min) to afford tert-butyl (R)-2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoate (12 mg, first peak) and tert-butyl (S)-2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoate (12 mg, second peak) both as white solid. Absolute configuration was arbitrarily assigned to each enantiomer. MS: m/z 529.2 (M+H⁺).

### Step 4—Synthesis of (R)-2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoic acid

To a mixture of tert-butyl (R)-2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoate (12 mg, 23 umol) in DCM (1 mL) was added TFA (0.5 mL). The reaction was stirred at room temperature for 16 hours. This mixture was concentrated in vacuo and then dissolved in MeOH (2 mL). The mixture was adjusted pH to 10 with aq. LiOH (2 M), then adjusted pH to 6 with formic acid. The solution was purified by reverse phase chromatography (acetonitrile 45-75%/0.225% formic acid in water) to give (R)-2-((1-(7-methyl-4-oxo-2-(2-oxo-2,3-dihydrobenzo[d]thiazol-5-yl)-4H-pyrido[1,2-a]pyrimidin-9-yl)ethyl)amino)benzoic acid (Compound 613) (4 mg, 35%) as a white solid. ¹H NMR (400 MHz, DMSO-d₆) δ 8.75 (s, 1H), 8.70-8.57 (m, 1H), 8.08 (s, 1H), 8.01 (d, J=8.4 Hz, 1H), 7.82 (d, J=6.8 Hz, 1H), 7.78 (d, J=1.2 Hz, 1H), 7.73 (d, J=8.4 Hz, 1H), 7.19-7.15 (m, 1H), 7.01 (s, 1H), 6.56-6.52 (m, 1H), 6.39 (d, J=8.8 Hz, 1H), 5.60-5.49 (m, 1H), 2.35 (s, 3H), 1.67 (d, J=6.4 Hz, 3H). MS: m/z 473.0 (M+H⁻).

### Example 19: Preparation of (R)-2-((1-(2-(4-fluorophenyl)-7-methyl-4-oxo-4H-pyrido[1,2-a]pyrimidin-9-yl)ethyl)amino)benzoic acid (Compound 424)

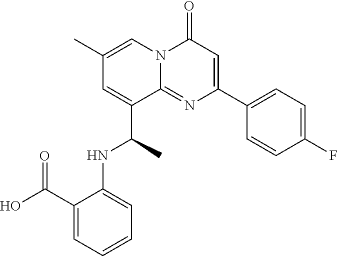 [SMILES: Cc1cc([C@@H](C)Nc2ccccc2C(=O)O)c2nc(-c3ccc(F)cc3)cc(=O)n2c1]

### Step 1—Synthesis of tert-butyl 2-((1-(2-(4-fluorophenyl)-7-methyl-4-oxo-4H-pyrido[1,2-a]pyrimidin-9-yl)ethyl)amino)benzoate

To a mixture of tert-butyl 2-((1-(7-methyl-4-oxo-2-(((trifluoromethyl)sulfonyl)oxy)-4H-pyrido[1,2-a]pyrimidin-9-yl)ethyl)amino)benzoate (100 mg, 189 umol), K₂CO₃ (79 mg, 0.56 mmol) and (4-fluorophenyl)boronic acid (53 mg, 379 umol) in dioxane (3 mL) and water (0.3 mL), Pd(PPh₃)₄ (22 mg, 19 umol) was added. The reaction mixture was heated to 100° C. and stirred for 2 hours under N₂ atmosphere. After cooling to room temperature, the reaction was diluted with water (20 mL) and EtOAc (20 mL×3). The combined organic layers were washed with brine (30 mL), dried over anhydrous Na₂SO₄, filtered and concentrated in vacuo. The crude residue was purified by silica gel chromatography (solvent gradient: 0-50% EtOAc in petroleum ether) to give tert-butyl 2-((1-(2-(4-fluorophenyl)-7-methyl-4-oxo-4H-pyrido[1,2-a]pyrimidin-9-yl)ethyl)amino)benzoate (72 mg, 80%) as a white solid. ¹H NMR (400 MHz, DMSO-d₆) 8.76 (s, 1H), 8.41-8.27 (m, 3H), 7.85-7.73 (m, 2H), 7.41-7.33 (m, 2H), 7.25-7.16 (m, 1H), 7.05 (s, 1H), 6.58-6.51 (m, 1H), 6.46 (d, J=8.4 Hz, 1H), 5.55-5.48 (m, 1H), 2.35 (s, 3H), 1.69 (d, J=6.8 Hz, 3H), 1.54 (s, 9H). MS: m/z 474.2 (M+H⁺).

### Step 2—Synthesis of 2-((1-(2-(4-fluorophenyl)-7-methyl-4-oxo-4H-pyrido[1,2-a]pyrimidin-9-yl)ethyl)amino)benzoic acid

To a mixture of tert-butyl 2-((1-(2-(4-fluorophenyl)-7-methyl-4-oxo-4H-pyrido[1,2-a]pyrimidin-9-yl)ethyl)amino)benzoate (70 mg, 148 umol) in DCM (1.5 mL) was added TFA (0.5 mL). The reaction mixture was stirred at room temperature for 16 hours. The mixture was concentrated in vacuo. The crude residue was purified by silica gel chromatography (solvent gradient: 0-3% MeOH in DCM) to give 2-((1-(2-(4-fluorophenyl)-7-methyl-4-oxo-4H-pyrido[1,2-a]pyrimidin-9-yl)ethyl)amino)benzoic acid (57 mg, 35%) as a yellow solid. ¹H NMR (400 MHz, DMSO-d₆) δ 12.77 (s, 1H), 8.76 (s, 1H), 8.50 (d, J=6.0 Hz, 1H), 8.39-8.32 (m, 2H), 7.85-7.75 (m, 2H), 7.41-7.33 (m, 2H), 7.24-7.15 (m, 1H), 7.06 (s, 1H), 6.58-6.51 (m, 1H), 6.41 (d, J=8.8 Hz, 1H), 5.60-5.51 (m, 1H), 2.34 (s, 3H), 1.67 (d, J=6.8 Hz, 3H). MS: m/z 418.0 (M+H⁺).

### Step 3—Synthesis of (R)-2-((1-(2-(4-fluorophenyl)-7-methyl-4-oxo-4H-pyrido[1,2-a]pyrimidin-9-yl)ethyl)amino)benzoic acid (Compound 438) and (S)-2-((1-(2-(4-fluorophenyl)-7-methyl-4-oxo-4H-pyrido[1,2-a]pyrimidin-9-yl)ethyl)amino)benzoic acid

2-((1-(2-(4-fluorophenyl)-7-methyl-4-oxo-4H-pyrido[1,2-a]pyrimidin-9-yl)ethyl)amino)benzoic acid (30 mg, 72 umol) was separated by using chiral SFC (DAICEL CHIRALCEL IC (250 mm*30 mm, 10 um); Supercritical CO₂/EtOH+0.1% NH₃·H₂O=50/50; 70 mL/min) to afford (R)-2-((1-(2-(4-fluorophenyl)-7-methyl-4-oxo-4H-pyrido[1,2-a]pyrimidin-9-yl)ethyl)amino)benzoic acid (10 mg, first peak) and (S)-2-((1-(2-(4-fluorophenyl)-7-methyl-4-oxo-4H-pyrido[1,2-a]pyrimidin-9-yl)ethyl)amino)benzoic acid (4 mg, second peak) both as white solid. Absolute configuration was arbitrarily assigned to each enantiomer. Compound 438: ¹H NMR (400 MHz, DMSO-d₆) δ 12.80 (s, 1H), 8.75 (s, 1H), 8.57-8.47 (m, 1H), 8.38-8.32 (m, 2H), 7.84-7.79 (m, 1H), 7.77 (s, 1H), 7.41-7.33 (m, 2H), 7.22-7.16 (m, 1H), 7.06 (s, 1H), 6.58-6.50 (m, 1H), 6.41 (d, J=8.4 Hz, 1H), 5.59-5.50 (m, 1H), 2.34 (s, 3H), 1.67 (d, J=6.8 Hz, 3H). MS: m/z 418.0 (M+H⁺).

### Example 20: Preparation of (R)-2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoic acid (Compound 696)

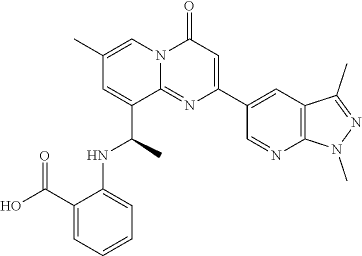 [SMILES: Cc1cc([C@@H](C)Nc2ccccc2C(=O)O)c2nc(-c3cnc4c(c3)c(C)nn4C)cc(=O)n2c1]

### Step 1—Synthesis of 1,3-dimethyl-5-(4,4,5,5-tetramethyl-1,3,2-dioxaborolan-2-yl)-1H-pyrazolo[3,4-b]pyridine

A mixture of 5-bromo-1,3-dimethyl-pyrazolo[3,4-b]pyridine (710 mg, 3.14 mmol) (prepared according to the procedure in WO2016123629), Pd(dppf)Cl₂ (230 mg, 314 umol), KOAc (925 mg, 9.42 mmol) and 4,4,5,5-tetramethyl-2-(4,4,5,5-tetramethyl-1,3,2-dioxaborolan-2-yl)-1,3,2-dioxaborolane (2.39 g, 9.42 mmol) in dioxane (8 mL) was stirred at 100° C. for 2 hours under N₂ atmosphere. After cooling to room temperature, the reaction was filtered and concentrated in vacuo. The crude residue was purified by silica gel chromatography (solvent gradient: 0-30% EtOAc in petroleum ether) to give 1,3-dimethyl-5-(4,4,5,5-tetramethyl-1,3,2-dioxaborolan-2-yl)-1H-pyrazolo[3,4-b]pyridine (700 mg, 82%) as a brown solid. MS: m/z 274.1 (M+H⁺).

### Step 2—Synthesis of tert-butyl 2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoate

To a solution of tert-butyl 2-((1-(7-methyl-4-oxo-2-(((trifluoromethyl)sulfonyl)oxy)-4H-pyrido[1,2-a]pyrimidin-9-yl)ethyl)amino)benzoate (193 mg, 366 umol), 1,3-dimethyl-5-(4,4,5,5-tetramethyl-1,3,2-dioxaborolan-2-yl)-1H-pyrazolo[3,4-b]pyridine (200 mg, 732 umol), K₂CO₃ (152 mg, 1.10 mmol) in dioxane (2 mL) and water (0.2 mL) was added Pd(PPh₃)₄ (42 mg, 37 umol). The reaction mixture was stirred at 100° C. for 2 hours under N₂ atmosphere. After cooling to room temperature, the mixture was diluted with EtOAc (60 mL), washed with brine (30 mL), dried over anhydrous Na₂SO₄, filtered and concentrated in vacuo. The crude residue was purified by column chromatography (solvent gradient: 0-30% EtOAc in petroleum ether) to give tert-butyl 2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoate (90 mg, 47%) as a brown solid. MS: m/z 525.1 (M+H⁺).

### Step 3—Synthesis of tert-butyl (R)-2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoate & tert-butyl (S)-2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoate

tert-butyl 2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoate (70 mg, 133 umol) was separated by using chiral SFC (DAICEL CHIRALCEL AD (250 mm*30 mm, 5 um); Supercritical CO₂/i-PrOH+0.1% NH₃·H₂O=70/30; 60 ml/min) to afford tert-butyl (R)-2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoate (20 mg, first peak) and tert-butyl (S)-2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoate (25 mg, second peak) both as white solid. Absolute configuration was arbitrarily assigned to each enantiomer. MS: m/z 525.1 (M+H⁻).

### Step 4—Synthesis of (R)-2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoic acid

To a mixture of tert-butyl (R)-2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoate (20 mg, 38 umol) in DCM (1 mL) was added TFA (0.5 mL). The reaction was stirred at room temperature for 16 h. The mixture was concentrated in vacuo and then diluted with MeOH (2 mL). The solution was adjusted pH to 10 with aq. LiOH (2 M), then adjusted pH to 6 with formic acid. The solution was purified by reverse phase chromatography (acetonitrile 30-70%/0.225% formic acid in water) to give (R)-2-((1-(2-(1,3-dimethyl-1H-pyrazolo[3,4-b]pyridin-5-yl)-7-methyl-4-oxo-4H-pyrido[1,2-a]pyrimidin-9-yl)ethyl)amino)benzoic acid (Compound 726) (7 mg, 23%) as a white solid. ¹H NMR (400 MHz, DMSO-d₆) δ 9.49 (d, J=2.0 Hz, 1H), 9.10 (d, J=2.0 Hz, 1H), 8.78 (s, 1H), 8.71-8.59 (m, 1H), 7.84-7.79 (m, 2H), 7.24 (s, 1H), 7.21-7.17 (m, 1H), 6.56-6.52 (m, 1H), 6.47 (d, J=8.4 Hz, 1H), 5.62-5.53 (m, 1H), 4.03 (s, 3H), 2.59 (s, 3H), 2.36 (s, 3H), 1.71 (d, J=6.4 Hz, 3H). MS: m/z 469.1 (M+H⁺).

### Example 21: Preparation of 2-((1-(2-(1,3-dimethyl-1H-pyrazolo[3,4-b]pyridin-5-yl)-3,7-dimethyl-4-oxo-4H-pyrido[1,2-a]pyrimidin-9-yl)ethyl)amino)benzoic acid (Compound 1411)

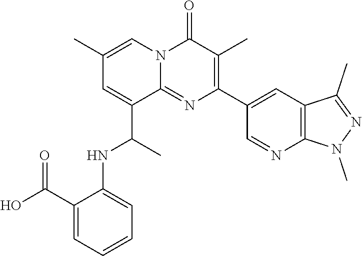 [SMILES: Cc1cc(C(C)Nc2ccccc2C(=O)O)c2nc(-c3cnc4c(c3)c(C)nn4C)c(C)c(=O)n2c1]

### Step 1—Synthesis of tert-butyl 2-((1-(2-(1,3-dimethyl-1H-pyrazolo[3,4-b]pyridin-5-yl)-3,7-dimethyl-4-oxo-4H-pyrido[1,2-a]pyrimidin-9-yl)ethyl)amino)benzoate

To a solution of tert-butyl 2-((1-(3,7-dimethyl-4-oxo-2-(((trifluoromethyl)sulfonyl)oxy)-4H-pyrido[1,2-a]pyrimidin-9-yl)ethyl)amino)benzoate (100 mg, 185 umol), 1,3-dimethyl-5-(4,4,5,5-tetramethyl-1,3,2-dioxaborolan-2-yl)-1H-pyrazolo[3,4-b]pyridine (101 mg, 369 umol) and K₂CO₃ (77 mg, 554 umol) in dioxane (2 mL) and water (0.2 mL) was added Pd(PPh₃)₄ (21 mg, 18 umol). The reaction mixture was stirred at 100° C. for 2 hours under N₂ atmosphere. After cooling to room temperature, the mixture was diluted with EtOAc (50 mL), washed with brine (30 mL), dried over anhydrous Na₂SO₄, filtered and concentrated in vacuo. The crude residue was purified by column chromatography (solvent gradient: 0-25% EtOAc in petroleum ether) to give the title compound (34 mg, 34%) as a brown solid. MS: m/z 539.1 (M+H⁺).

### Step 2—Synthesis of 2-((1-(2-(1,3-dimethyl-1H-pyrazolo[3,4-b]pyridin-5-yl)-3,7-dimethyl-4-oxo-4H-pyrido[1,2-a]pyrimidin-9-yl)ethyl)amino)benzoic acid

To a mixture of tert-butyl 2-((1-(2-(1,3-dimethyl-1H-pyrazolo[3,4-b]pyridin-5-yl)-3,7-dimethyl-4-oxo-4H-pyrido[1,2-a]pyrimidin-9-yl)ethyl)amino)benzoate (34 mg, 63 umol) in DCM (3 mL) was added TFA (1 mL). The reaction was stirred at room temperature for 16 h. This mixture was concentrated in vacuo and then diluted with MeOH (2 mL). The solution was adjusted pH to 10 with aq. LiOH (2 M), then adjusted pH to 6 with formic acid. The solution was purified by reverse phase chromatography (acetonitrile 50-80%/0.225% formic acid in water) to give 2-((1-(2-(1,3-dimethyl-1H-pyrazolo[3,4-b]pyridin-5-yl)-3,7-dimethyl-4-oxo-4H-pyrido[1,2-a]pyrimidin-9-yl)ethyl)amino)benzoic acid (Compound 752) (11 mg, 36%) as a white solid. ¹H NMR (400 MHz, DMSO-d₆) δ 8.94 (d, J=2.0 Hz, 1H), 8.72 (s, 1H), 8.56 (d, J=2.0 Hz, 1H), 8.51 (s, 1H), 7.81 (dd, J=8.0, 1.6 Hz, 1H), 7.68 (d, J=1.6 Hz, 1H), 7.20-7.16 (m, 1H), 6.55-7.51 (m, 1H), 6.39 (d, J=8.4 Hz, 1H), 5.48-5.34 (m, 1H), 4.03 (s, 3H), 2.56 (s, 3H), 2.34 (s, 3H), 2.30 (s, 3H), 1.62 (d, J=6.4 Hz, 3H). MS: m/z 483.1 (M+H⁺).

### Example 22: Preparation of 2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoic acid (Compound 1410)

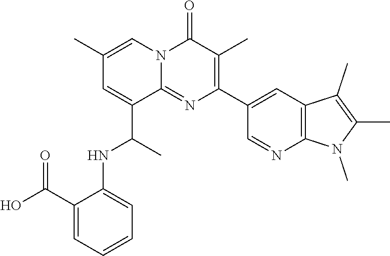 [SMILES: Cc1cc(C(C)Nc2ccccc2C(=O)O)c2nc(-c3cnc4c(c3)c(C)c(C)n4C)c(C)c(=O)n2c1]

### Step 1—Synthesis of 5-bromo-1,2,3-trimethyl-1H-pyrrolo[2,3-b]pyridine

To a solution of 5-bromo-2,3-dimethyl-1H-pyrrolo[2,3-b]pyridine (1.11 g, 4.93 mmol) (prepared according to the procedure in US201182138) in DMF (10 mL) was added NaH (237 mg, 5.92 mmol, 60% purity) and Mel (338 uL, 5.42 mmol) under N₂ atmosphere at 0° C. The mixture was stirred at 0° C. for 2 hours. The reaction was quenched with sat. aq. NH₄Cl (20 mL), diluted with water (20 mL), extracted with EtOAc (60 mL×2). The combined organic layers were washed with brine (30 mL×3), dried over anhydrous Na₂SO₄, filtered and concentrated in vacuo. The crude residue was purified by silica gel chromatography (solvent gradient: 0-20% EtOAc in petroleum ether) to give 5-bromo-1,2,3-trimethyl-1H-pyrrolo[2,3-b]pyridine (1.1 g, 93%) as a yellow solid. ¹H NMR (400 MHz, CDCl₃) δ 8.23 (d, J=2.0 Hz, 1H), 7.85 (d, J=2.0 Hz, 1H), 3.75 (s, 3H), 2.38 (s, 3H), 2.20 (s, 3H). MS: m/z 241.0 (M+H⁺).

### Step 2—Synthesis of 1,2,3-trimethyl-5-(4,4,5,5-tetramethyl-1,3,2-dioxaborolan-2-yl)-1H-pyrrolo[2,3-b]pyridine

To a solution of 5-bromo-1,2,3-trimethyl-pyrrolo[2,3-b]pyridine (1 g, 4.18 mmol) in 1,4-dioxane (15 mL) was added Pd(dppf)Cl₂ (306 mg, 418.22 μmol), KOAc (1.23 g, 12.55 mmol) and 4,4,5,5-tetramethyl-2-(4,4,5,5-tetramethyl-1,3,2-dioxaborolan-2-yl)-1,3,2-dioxaborolane (3.19 g, 12.55 mmol). The reaction mixture was stirred at 80° C. for 16 hours under N₂ atmosphere. After cooling to room temperature, EtOAc (100 mL) was added, and the mixture was washed with water (50 mL) and brine (50 mL). The organic layer was dried over anhydrous Na₂SO₄, filtered and concentrated in vacuo. The crude residue was purified by silica gel chromatography (solvent gradient: 0-25% EtOAc in petroleum ether) to give 1,2,3-trimethyl-5-(4,4,5,5-tetramethyl-1,3,2-dioxaborolan-2-yl)-1H-pyrrolo[2,3-b]pyridine (1.14 g, 95%) as a white solid. ¹H NMR (400 MHz, DMSO-d₆) δ 8.38 (d, J=1.6 Hz, 1H), 8.04 (d, J=1.6 Hz, 1H), 3.70 (s, 3H), 2.36 (s, 3H), 2.20 (s, 3H), 1.31 (s, 12H). MS: m/z 287.2 (M+H⁺).

### Step 3—Synthesis of tert-butyl 2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoate

To a solution of tert-butyl 2-((1-(3,7-dimethyl-4-oxo-2-(((trifluoromethyl)sulfonyl)oxy)-4H-pyrido[1,2-a]pyrimidin-9-yl)ethyl)amino)benzoate (190 mg, 349 mol), K₂CO₃ (145 mg, 1.05 mmol) and 1,2,3-trimethyl-5-(4,4,5,5-tetramethyl-1,3,2-dioxaborolan-2-yl)-1H-pyrrolo[2,3-b]pyridine (200 mg, 699 mol) in dioxane (2 mL) and water (0.2 mL) was added Pd(PPh₃)₄ (40 mg, 35 mol). The reaction was stirred at 100° C. for 2 hours under N₂ atmosphere. After cooling to room temperature, the reaction was filtered and concentrated in vacuo. The crude residue was purified by silica gel chromatography (solvent gradient: 0-5% MeOH in DCM) to give the title compound (190 mg, 98%) as a white solid. MS: m/z 552.2 (M+H⁺).

### Step 4—Synthesis of 2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoic acid

To a mixture of tert-butyl 2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoate (13 mg, 24 umol) in DCM (1 mL) was added TFA (0.5 mL). The reaction was stirred at room temperature for 16 hours. This mixture was concentrated in vacuo and then diluted with MeOH (2 mL). The solution was adjusted pH to 10 with aq. LiOH (2 M), then adjusted pH to 6 with formic acid. The solution was purified by reverse phase chromatography (acetonitrile 50-80%/0.225% formic acid in water) to give 2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoic acid (Compound 798) (3 mg, 25%) as a white solid. ¹H NMR (400 MHz, DMSO-d₆) δ 8.71 (s, 1H), 8.56 (d, J=2.0 Hz, 1H), 8.44 (d, J=6.4 Hz, 1H), 8.16 (d, J=2.0 Hz, 1H), 7.81-7.79 (m, 1H), 7.65 (d, J=1.6 Hz, 1H), 7.23-7.17 (m, 1H), 6.56-6.52 (m, 1H), 6.39 (d, J=8.4 Hz, 1H), 5.46-5.40 (m, 1H), 3.75 (s, 3H), 2.41 (s, 3H), 2.33 (s, 3H), 2.31 (s, 3H), 2.24 (s, 3H), 1.62 (d, J=6.4 Hz, 3H). MS: m/z 496.2 (M+H⁺).

### Example 23: Preaparation of 2-(isoindolin-2-yl)-7-methyl-9-(1-((2-(pyridazin-4-yl)phenyl)amino)ethyl)-4H-pyrido[1,2-a]pyrimidin-4-one (Compound 665)

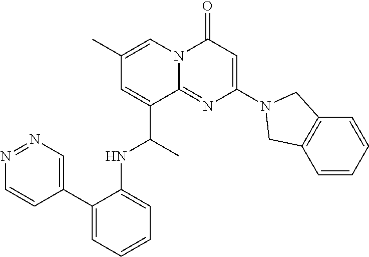 [SMILES: Cc1cc(C(C)Nc2ccccc2-c2ccnnc2)c2nc(N3Cc4ccccc4C3)cc(=O)n2c1]

### Step 1—Synthesis of 9-(1-((2-bromophenyl)amino)ethyl)-2-(isoindolin-2-yl)-7-methyl-4H-pyrido[1,2-a]pyrimidin-4-one

A mixture of 9-(1-bromoethyl)-2-(isoindolin-2-yl)-7-methyl-4H-pyrido[1,2-a]pyrimidin-4-one (50 mg, 0.13 mmol) and 2-bromoaniline (112 mg, 0.65 mmol) in dioxane (1 mL) was heated to 100° C. for 2 hours under N₂ atmosphere. After cooling to room temperature, the mixture was concentrated in vacuo. The crude residue was purified by silica gel chromatography (solvent gradient: 0-30% EtOAc in petroleum ether) to give 9-(1-((2-bromophenyl)amino)ethyl)-2-(isoindolin-2-yl)-7-methyl-4H-pyrido[1,2-a]pyrimidin-4-one (370 mg, 59%) as colorless oil. ¹H NMR (400 MHz, CDCl₃) δ 8.72 (s, 1H), 7.53 (s, 1H), 7.45 (d, J=8.0 Hz, 1H), 7.40-7.31 (m, 4H), 7.03-6.98 (m, 1H), 6.59-6.52 (m, 1H), 6.34 (d, J=8.0 Hz, 1H), 5.62 (s, 1H), 5.38-5.25 (m, 1H), 5.17-4.97 (m, 2H), 4.83-4.67 (m, 2H), 4.16-4.10 (m, 1H), 2.30 (s, 3H), 1.73 (br d, J=6.8 Hz, 3H). MS: m/z 475.2 (M+H⁺).

### Step 2—Synthesis of 2-(isoindolin-2-yl)-7-methyl-9-(1-((2-(pyridazin-4-yl)phenyl)amino)ethyl)-4H-pyrido[1,2-a]pyrimidin-4-one

To a solution of 9-(1-((2-bromophenyl)amino)ethyl)-2-(isoindolin-2-yl)-7-methyl-4H-pyrido[1,2-a]pyrimidin-4-one (200 mg, 0.42 mmol), Na₂CO₃ (134 mg, 1.26 mmol) and 4-(tributylstannyl)pyridazine (163 mg, 0.44 mmol) in 1,4-dioxane (2 mL) was added Pd(dppf)Cl₂ (31 mg, 0.04 mmol). The reaction was stirred at 100° C. for 16 hours under N₂ atmosphere. After cooling to room temperature, the reaction was filtered and concentrated in vacuo. The crude residue was purified by reverse phase chromatography (acetonitrile 55-85%/formic acid in water) to give 2-(isoindolin-2-yl)-7-methyl-9-(1-((2-(pyridazin-4-yl)phenyl)amino)ethyl)-4H-pyrido[1,2-a]pyrimidin-4-one (Compound 694) (16 mg, 8%) as a white solid. ¹H NMR (400 MHz, DMSO-d₆) δ 9.40 (s, 1H), 7.23 (d, J=5.2 Hz, 1H), 8.57 (s, 1H), 7.88 (d, J=8.0 Hz, 1H), 7.81 (s, 1H), 7.41-7.40 (m, 1H), 7.34-7.32 (m, 1H), 7.14-7.10 (m, 1H), 6.81-6.79 (m, 1H), 6.55-6.50 (m, 1H), 5.84 (d, J=7.6 Hz, 1H), 5.40 (s, 1H), 5.27-5.25 (m, 1H), 4.89-4.85 (m, 2H), 4.72-4.58 (m, 3H), 2.26 (s, 3H), 1.53 (d, J=6.4 Hz, 3H). MS: m/z 475.0 (M+H⁺).

### Example 24: Preparation of 2-[[(1R)-1-[2-[(2S,6R)-2,6-dimethylmorpholin-4-yl]-7-methyl-4-oxo-pyrido[1,2-a]pyrimidin-9-yl]ethyl]amino]benzenesulfonamide (Compound 730)

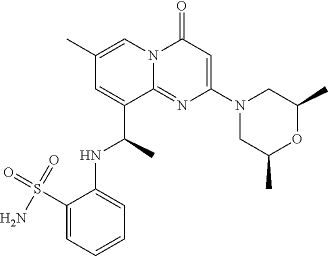 [SMILES: Cc1cc([C@@H](C)Nc2ccccc2S(N)(=O)=O)c2nc(N3C[C@@H](C)O[C@@H](C)C3)cc(=O)n2c1]

### Step 1—Synthesis of 2-[1-[2-[(2S,6R)-2,6-dimethylmorpholin-4-yl]-7-methyl-4-oxo-pyrido[1,2-a]pyrimidin-9-yl]ethylamino]benzenesulfonamide

To a solution of 2-[1-(2-hydroxy-7-methyl-4-oxo-pyrido[1,2-a]pyrimidin-9-yl)ethylamino]benzenesulfonamide (200 mg, 534 umol) in DMF (1 mL) was added PyBOP (334 mg, 641 umol) and DIEA (465 uL, 2.67 mmol). The mixture was stirred at room temperature for 20 min, (2S,6R)-2,6-dimethylmorpholine (92 mg, 801 umol) was added. The reaction was stirred at 40° C. for 16 hours. After cooling to room temperature, the reaction mixture was quenched with water (20 mL), extracted with EtOAc (50 mL×2). The combined organic layers were washed with aq. HCl (1 M, 20 mL) and brine (20 mL), dried over anhydrous Na₂SO₄, filtered and concentrated in vacuo. The resulting residue was purified by Prep-TLC (10% MeOH in DCM) to give 2-[1-[2-[(2S,6R)-2,6-dimethylmorpholin-4-yl]-7-methyl-4-oxo-pyrido[1,2-a]pyrimidin-9-yl]ethylamino]benzenesulfonamide (80 mg, 32%) as a white solid. ¹H NMR (400 MHz, DMSO-d₆) δ 8.57 (s, 1H), 7.73 (d, J=2.0 Hz, 1H), 7.64 (d, J=6.4 Hz, 1H), 7.54-7.51 (m, 1H), 7.23-7.20 (m, 2H), 6.78 (d, J=8.4 Hz, 1H), 6.43-6.36 (m, 2H), 5.83 (s, 1H), 5.76 (s, 1H), 5.29-5.22 (m, 1H), 3.63-3.58 (m, 2H), 2.60-2.55 (m, 4H), 2.23 (s, 3H), 1.57 (d, J=6.4 Hz, 3H), 1.27-1.21 (m, 6H). MS: m/z 472.2 (M+H⁺).

### Step 2—Synthesis of 2-[[(1R)-1-[2-[(2S,6R)-2,6-dimethylmorpholin-4-yl]-7-methyl-4-oxo-pyrido[1,2-a]pyrimidin-9-yl]ethyl]amino]benzenesulfonamide & 2-[[(1S)-1-[2-[(2S,6R)-2,6-dimethylmorpholin-4-yl]-7-methyl-4-oxo-pyrido[1,2-a]pyrimidin-9-yl]ethyl]amino]benzenesulfonamide

2-[1-[2-[(2S,6R)-2,6-dimethylmorpholin-4-yl]-7-methyl-4-oxo-pyrido[1,2-a]pyrimidin-9-yl]ethylamino]benzenesulfonamide (60 mg, 127 umol) was separated by using chiral SFC (Phenomenex-Cellulose-2 (250 mm*30 mm, 10 um); Supercritical CO₂/EtOH+0.1% NH₃·H₂O=50/50; 80 mL/min) to afford 2-[[(1R)-1-[2-[(2S,6R)-2,6-dimethylmorpholin-4-yl]-7-methyl-4-oxo-pyrido[1,2-a]pyrimidin-9-yl]ethyl]amino]benzenesulfonamide (30 mg, first peak) and 2-[[(1S)-1-[2-[(2S,6R)-2,6-dimethylmorpholin-4-yl]-7-methyl-4-oxo-pyrido[1,2-a]pyrimidin-9-yl]ethyl]amino]benzenesulfonamide (30 mg, second peak) both as white solid. Absolute configuration was arbitrarily assigned to each enantiomer. Compound 25: ¹H NMR (400 MHz, DMSO-d₆) δ 8.54 (s, 1H), 7.73 (s, 1H), 7.64 (d, J=7.6 Hz, 1H), 7.51 (s, 2H), 7.26-7.19 (m, 1H), 6.71-6.60 (m, 1H), 6.43-6.34 (m, 2H), 5.70 (s, 1H), 5.33-5.17 (m, 1H), 4.45-4.19 (m, 2H), 3.64-3.57 (m, 2H), 3.49-3.42 (m, 2H), 2.23 (s, 3H), 1.57 (d, J=6.4 Hz, 3H), 1.17-1.12 (m, 6H). MS: m/z 472.2 (M+H⁻).

### Example 25: Preparation of (R)-2-((1-(2-(4,4-dimethylcyclohexyl)-7-methyl-4-oxo-4H-pyrido[1,2-a]pyrimidin-9-yl)ethyl)amino)benzoic acid (Compound 623)

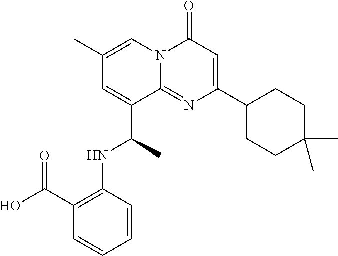 [SMILES: Cc1cc([C@@H](C)Nc2ccccc2C(=O)O)c2nc(C3CCC(C)(C)CC3)cc(=O)n2c1]

### Step 1—Synthesis of tert-butyl 2-((1-(2-(4,4-dimethylcyclohex-1-en-1-yl)-7-methyl-4-oxo-4H-pyrido[1,2-a]pyrimidin-9-yl)ethyl)amino)benzoate

A mixture of tert-butyl 2-((1-(7-methyl-4-oxo-2-(((trifluoromethyl)sulfonyl)oxy)-4H-pyrido[1,2-a]pyrimidin-9-yl)ethyl)amino)benzoate (220 mg, 417 umol), 2-(4,4-dimethylcyclohexen-1-yl)-4,4,5,5-tetramethyl-1,3,2-dioxaborolane (148 mg, 625.58 umol), Pd(PPh₃)₄ (48 mg, 42 umol), K₂CO₃ (173 mg, 1.25 mmol) in dioxane (2 mL) and water (0.5 mL) was stirred at 100° C. for 2 hours under N₂ atmosphere. The mixture was filtered and concentrated in vacuo. The crude residue was purified by silica gel chromatography (solvent gradient: 0-20% EtOAc in petroleum ether) to give tert-butyl 2-((1-(2-(4,4-dimethylcyclohex-1-en-1-yl)-7-methyl-4-oxo-4H-pyrido[1,2-a]pyrimidin-9-yl)ethyl)amino)benzoate (190 mg, 93%) as a yellow solid. ¹H NMR (400 MHz, DMSO-d₆) δ 8.67 (s, 1H), 8.26 (d, J=6.8 Hz, 1H), 7.79-7.75 (m, 1H), 7.74-7.71 (m, 1H), 7.24-7.20 (m, 1H), 7.15 (s, 1H), 6.57-6.53 (m, 1H), 6.48-6.39 (m, 2H), 5.45-5.24 (m, 1H), 2.32 (s, 3H), 2.09 (s, 2H), 1.65 (d, J=6.8 Hz, 3H), 1.55 (s, 9H), 1.52 (t, J=6.0 Hz, 2H), 1.31-1.22 (m, 4H), 0.97-0.94 (m, 6H). MS: m/z 488.1 (M+H⁺).

### Step 2—Synthesis of tert-butyl 2-((1-(2-(4,4-dimethylcyclohexyl)-7-methyl-4-oxo-4H-pyrido[1,2-a]pyrimidin-9-yl)ethyl)amino)benzoate

To a solution of tert-butyl 2-((1-(2-(4,4-dimethylcyclohex-1-en-1-yl)-7-methyl-4-oxo-4H-pyrido[1,2-a]pyrimidin-9-yl)ethyl)amino)benzoate (150 mg, 300 umol) in MeOH (10 mL) was added wet Pd/C (0.2 g, 10% Pd, 50% wet with water). The reaction was stirred at room temperature for 2 h under H2 atmosphere (15 psi). The reaction was filtered through diatomaceous earth and the filtrate was concentrated in vacuo to afford tert-butyl 2-((1-(2-(4,4-dimethylcyclohexyl)-7-methyl-4-oxo-4H-pyrido[1,2-a]pyrimidin-9-yl)ethyl)amino)benzoate (150 mg, crude) as yellow oil that required no further purification. MS: m/z 490.2 (M+H⁺).

### Step 3—Synthesis of 2-((1-(2-(4,4-dimethylcyclohexyl)-7-methyl-4-oxo-4H-pyrido[1,2-a]pyrimidin-9-yl)ethyl)amino)benzoic acid

To a solution of tert-butyl 2-((1-(2-(4,4-dimethylcyclohexyl)-7-methyl-4-oxo-4H-pyrido[1,2-a]pyrimidin-9-yl)ethyl)amino)benzoate (100 mg, 204 umol) in DCM (3 mL) was added TFA (1 mL). The reaction mixture was stirred at room temperature for 16 hours. The mixture was concentrated in vacuo. The residue was purified by reverse phase chromatography (acetonitrile 80%-100%/0.225% formic acid in water) to give 2-((1-(2-(4,4-dimethylcyclohexyl)-7-methyl-4-oxo-4H-pyrido[1,2-a]pyrimidin-9-yl)ethyl)amino)benzoic acid (45 mg, 50%) as a white solid. ¹H NMR (400 MHz, DMSO-d₆) δ 12.71 (s, 1H), 8.69 (s, 1H), 8.45 (s, 1H), 7.81 (d, J=6.8 Hz, 1H), 7.71 (s, 1H), 7.24-7.16 (m, 1H), 6.58-6.51 (m, 1H), 6.41-6.36 (m, 1H), 6.35-6.31 (m, 1H), 5.47-5.36 (m, 1H), 2.32 (s, 3H), 1.83-1.73 (m, 4H), 1.61 (d, J=6.4 Hz, 3H), 1.53-1.45 (m, 2H), 1.39-1.30 (m, 2H), 0.98-0.93 (m, 6H). MS: m/z 434.1 (M+H⁺).

### Step 4—Synthesis of (R)-2-((1-(2-(4,4-dimethylcyclohexyl)-7-methyl-4-oxo-4H-pyrido[1,2-a]pyrimidin-9-yl)ethyl)amino)benzoic acid & (S)-2-((1-(2-(4,4-dimethylcyclohexyl)-7-methyl-4-oxo-4H-pyrido[1,2-a]pyrimidin-9-yl)ethyl)amino)benzoic acid

2-((1-(2-(4,4-dimethylcyclohexyl)-7-methyl-4-oxo-4H-pyrido[1,2-a]pyrimidin-9-yl)ethyl)amino)benzoic acid (40 mg, 85 umol) was separated by using chiral SFC (DAICEL CHIRALCEL OJ (250 mm*30 mm, 10 um); Supercritical CO₂/i-PrOH+0.1% NH₃·H₂O=65/35; 80 mL/min) to afford (R)-2-((1-(2-(4,4-dimethylcyclohexyl)-7-methyl-4-oxo-4H-pyrido[1,2-a]pyrimidin-9-yl)ethyl)amino)benzoic acid (5 mg, first peak) and (S)-2-((1-(2-(4,4-dimethylcyclohexyl)-7-methyl-4-oxo-4H-pyrido[1,2-a]pyrimidin-9-yl)ethyl)amino)benzoic acid (5 mg, second peak) both as white solid. Absolute configuration was arbitrarily assigned to each enantiomer. Compound 649: ¹H NMR (400 MHz, DMSO-d₆) δ 12.73 (s, 1H), 8.69 (s, 1H), 8.44 (s, 1H), 7.81 (d, J=8.0 Hz, 1H), 7.75-7.70 (m, 1H), 7.24-7.16 (m, 1H), 6.58-6.51 (m, 1H), 6.41-6.36 (m, 1H), 6.33 (s, 1H), 5.47-5.36 (m, 1H), 2.32 (s, 3H), 1.83-1.73 (m, 4H), 1.61 (d, J=6.4 Hz, 3H), 1.53-1.45 (m, 2H), 1.39-1.30 (m, 2H), 0.98-0.93 (m, 6H). MS: m/z 434.1 (M+H⁺).

### Example 26: Preparation of 2-(((R)-1-(2-((3R,5S)-3,5-dimethylpiperidin-1-yl)-7-methyl-4-oxo-4H-pyrido[1,2-a]pyrimidin-9-yl)ethyl)amino)benzoic acid (Compound 512)

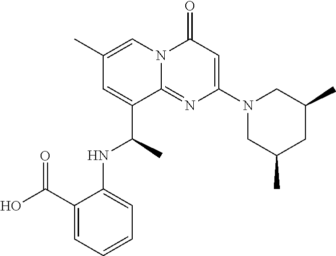 [SMILES: Cc1cc([C@@H](C)Nc2ccccc2C(=O)O)c2nc(N3C[C@H](C)C[C@H](C)C3)cc(=O)n2c1]

### Step 1—Synthesis of tert-butyl 2-((1-(2-((3R,5S)-3,5-dimethylpiperidin-1-yl)-7-methyl-4-oxo-4H-pyrido[1,2-a]pyrimidin-9-yl)ethyl)amino)benzoate

To a solution of tert-butyl 2-((1-(2-hydroxy-7-methyl-4-oxo-4H-pyrido[1,2-a]pyrimidin-9-yl)ethyl)amino)benzoate (100 mg, 0.25 mmol) in DMF (2 mL) was added PyBOP (158 mg, 0.30 mmol), DIEA (0.18 mL, 1.01 mmol). The mixture was stirred at room temperature for 30 minutes, (3R,5S)-3,5-dimethylpiperidine (114 mg, 1.01 mmol) was added. The reaction was stirred at 40° C. for 16 hours. After cooling to room temperature, the reaction mixture was quenched with water (20 mL), extracted with EtOAc (50 mL×2). The combined organic layers were washed with aq. HCl (1 M, 20 mL) and brine (20 mL×3), dried over anhydrous Na₂SO₄, filtered and concentrated in vacuo. The crude residue was purified by silica gel chromatography (solvent gradient: 0-20% EtOAc in petroleum ether) to give tert-butyl 2-((1-(2-((3R,5S)-3,5-dimethylpiperidin-1-yl)-7-methyl-4-oxo-4H-pyrido[1,2-a]pyrimidin-9-yl)ethyl)amino)benzoate (80 mg, 64%) as a white solid. ¹H NMR (400 MHz, DMSO-d₆) δ 8.51 (s, 1H), 8.18 (d, J=6.4 Hz, 1H), 7.78-7.76 (m, 1H), 7.59 (d, J=1.6 Hz, 1H), 7.31-7.15 (m, 1H), 6.55 (t, J=7.2 Hz, 1H), 6.39 (d, J=8.8 Hz, 1H), 5.67 (s, 1H), 5.23-5.16 (m, 1H), 4.46-4.36 (m, 2H), 2.47-2.34 (m, 4H), 2.23 (s, 3H), 1.80-1.75 (m, 1H), 1.60 (d, J=6.8 Hz, 3H), 1.57 (s, 9H), 0.89 (t, J=6.4 Hz, 6H), 0.86-0.81 (m, 1H) MS: m/z 491.2 (M+H⁺).

### Step 2—Synthesis of tert-butyl 2-(((R)-1-(2-((3R,5S)-3,5-dimethylpiperidin-1-yl)-7-methyl-4-oxo-4H-pyrido[1,2-a]pyrimidin-9-yl)ethyl)amino)benzoate and tert-butyl 2-(((S)-1-(2-((3R,5S)-3,5-dimethylpiperidin-1-yl)-7-methyl-4-oxo-4H-pyrido[1,2-a]pyrimidin-9-yl)ethyl)amino)benzoate

tert-butyl 2-((1-(2-((3R,5S)-3,5-dimethylpiperidin-1-yl)-7-methyl-4-oxo-4H-pyrido[1,2-a]pyrimidin-9-yl)ethyl)amino)benzoate (80 mg, 137 umol) was separated by prep SFC (REGIS(S,S)WHELK-O1 (250 mm*25 mm, 10 um), Supercritical CO₂/EtOH+0.1% NH₃·H₂O=55/45, 80 mL/min) to give tert-butyl 2-(((R)-1-(2-((3R,5S)-3,5-dimethylpiperidin-1-yl)-7-methyl-4-oxo-4H-pyrido[1,2-a]pyrimidin-9-yl)ethyl)amino)benzoate (35 mg, first peak) and tert-butyl 2-(((S)-1-(2-((3R,5S)-3,5-dimethylpiperidin-1-yl)-7-methyl-4-oxo-4H-pyrido[1,2-a]pyriimidin-9-yl)ethyl)amino)benzoate (35 mg, second peak) both as white solid. Absolute configuration was arbitrarily assigned to each enantiomer. MS: m/z 491.2 (M+H⁻).

### Step 3—Synthesis of 2-(((R)-1-(2-((3R,5S)-3,5-dimethylpiperidin-1-yl)-7-methyl-4-oxo-4H-pyrido[1,2-a]pyrimidin-9-yl)ethyl)amino)benzoic acid

To a mixture of tert-butyl 2-(((R)-1-(2-((3R,5S)-3,5-dimethylpiperidin-1-yl)-7-methyl-4-oxo-4H-pyrido[1,2-a]pyrimidin-9-yl)ethyl)amino)benzoate (35 mg, 71 umol) in DCM (1.5 mL) was added TFA (0.5 mL). The reaction was stirred at room temperature for 16 hours. This mixture was concentrated in vacuo and then diluted with MeOH (2 mL). The solution was adjusted pH to 10 with aq. LiOH (2 M), then adjusted pH to 6 with formic acid. The solution was purified by reverse phase chromatography (acetonitrile 65-95%/0.225% formic acid in water) to give 2-(((R)-1-(2-((3R,5S)-3,5-dimethylpiperidin-1-yl)-7-methyl-4-oxo-4H-pyrido[1,2-a]pyrimidin-9-yl)ethyl)amino)benzoic acid (Compound 529) (10 mg, 33%) as a white solid. Compound 529: ¹H NMR (400 MHz, DMSO-d₆) δ 12.73 (s, 1H), 8.51 (s, 1H), 8.40 (d, J=5.6 Hz, 1H), 7.82-7.80 (m, 1H), 7.56 (s, 1H), 7.25-7.21 (m, 1H), 6.57-6.53 (m, 1H), 6.36 (d, J=8.8 Hz, 1H), 5.67 (s, 1H), 5.23-5.19 (m, 1H), 4.55-4.25 (m, 2H), 2.44-2.36 (m, 2H), 2.22 (s, 3H), 1.82-1.78 (m, 1H), 1.61-1.55 (m, 5H), 0.90-0.88 (m, 6H), 0.87-0.79 (m, 1H). MS: m/z 435.1 (M+H⁺).

### Example 27: Preparation of (R)-2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoic acid (Compound 804)

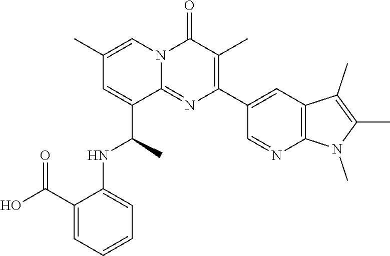 [SMILES: Cc1cc([C@@H](C)Nc2ccccc2C(=O)O)c2nc(-c3cnc4c(c3)c(C)c(C)n4C)c(C)c(=O)n2c1]

### Step 1—Synthesis of tert-butyl (R)-2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoate & tert-butyl (S)-2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoate

tert-butyl 2-[1-[3,7-dimethyl-4-oxo-2-(1,2,3-trimethylpyrrolo[2,3-b]pyridin-5-yl)pyrido[1,2-a]pyriimidin-9-yl]ethylaamino]benzoate (190 mg, 344 mol) was separated by using chiral SFC (DAICEL CHIRALPAK AD (250 mm*30 mm, 10 um); Supercritical CO₂/EtOH+0.1% NH₃·H₂O=80/20; 80 ml/min) to afford tert-butyl (R)-2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoate (75 mg, first peak) and tert-butyl (S)-2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoate (83 mg, second peak) both as white solid. Absolute configuration was arbitrarily assigned to each enantiomer. MS: m/z 552.3 (M+H⁺).

### Step 2—Synthesis of (R)-2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoic acid

To a mixture of tert-butyl (R)-2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoate (75 mg, 136 μmol) in DCM (3 mL) was added TFA (1 mL). The reaction mixture was stirred at room temperature for 16 hours. The mixture was concentrated in vacuo. The residue was purified by reverse phase chromatography (acetonitrile 60-90%/0.225% formic acid in water) to give (R)-2-((1-(3,7-dimethyl-4-oxo-2-(1,2,3-trimethyl-1H-pyrrolo[2,3-b]pyridin-5-yl)-4H-pyrido[1,2-a]pyrimidin-9-yl)ethyl)amino)benzoic acid (Compound 850) (50 mg, 74%) as a white solid. ¹H NMR (400 MHz, DMSO-d₆) δ 12.75 (s, 1H), 8.71 (s, 1H), 8.57 (d, J=1.6 Hz, 1H), 8.44 (d, J=5.4 Hz, 1H), 8.17 (d, J=1.6 Hz, 1H), 7.81 (d, J=8.0 Hz, 1H), 7.65 (s, 1H), 7.22-7.18 (m, 1H), 6.54 (s, 1H), 6.39 (d, J=8.4 Hz, 1H), 5.47-5.40 (m, 1H), 3.76 (s, 3H), 2.41 (s, 3H), 2.34 (s, 3H), 2.31 (s, 3H), 2.25 (s, 3H), 1.62 (d, J=6.4 Hz, 3H). MS: m/z 496.1 (M+H⁺).

### Example 28: Preparation of (S)-2-(isoindolin-2-yl)-3,7-dimethyl-9-(1-((2-(pyridazin-4-yl)phenyl)amino)ethyl)-4H-pyrido[1,2-a]pyrimidin-4-one (Compound 848)

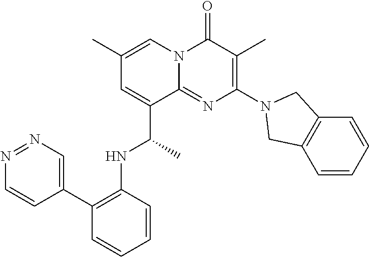 [SMILES: Cc1cc([C@H](C)Nc2ccccc2-c2ccnnc2)c2nc(N3Cc4ccccc4C3)c(C)c(=O)n2c1]

### Step 1—Synthesis of 9-bromo-2-(isoindolin-2-yl)-3,7-dimethyl-4H-pyrido[1,2-a]pyrimidin-4-one

To a solution of 9-bromo-2-hydroxy-3,7-dimethyl-pyrido[1,2-a]pyrimidin-4-one (10 g, 37.2 mmol) in DMF (100 mL) was added PyBOP (25.1 g, 48.3 mmol) and DIEA (25.9 mL, 148.7 mmol). The mixture was stirred at room temperature for 30 minutes, isoindoline hydrochloride (6.9 g, 44.6 mmol) was added. The reaction was stirred at 90° C. for 16 hours. After cooling to room temperature, 1M aq. HCl (200 mL) solution was added and the suspension solution was filtered and the filter cake was dried in vacuo to give 9-bromo-2-(isoindolin-2-yl)-3,7-dimethyl-4H-pyrido[1,2-a]pyrimidin-4-one (9.1 g, crude) as a yellow solid that required no further purification. ¹H NMR (400 MHz, DMSO-d₆) δ 8.63 (s, 1H), 8.14 (s, 1H), 7.42-7.37 (m, 2H), 7.33-7.29 (m, 2H), 5.16 (s, 4H), 2.40-2.37 (s, 3H), 2.33-2.31 (s, 3H). MS: m/z 371.8 (M+H⁺).

### Step 2—Synthesis of 9-acetyl-2-(isoindolin-2-yl)-3,7-dimethyl-4H-pyrido[1,2-a]pyrimidin-4-one

To a solution of 9-bromo-2-isoindolin-2-yl-3,7-dimethyl-pyrido[1,2-a]pyrimidin-4-one (5.2 g, 14.0 mmol) in dioxane (60 mL) was added Pd(dppf)Cl₂ (514 mg, 702 umol) and tributyl(1-ethoxyvinyl)stannane (9.5 mL, 28.0 mmol). The mixture was stirred at 90° C. for 16 hours under N₂ atmosphere. After cooling to room temperature, HCl (15 mL, 1 M) was added. The mixture was stirred at room temperature for 0.5 hours. The reaction mixture was added 100 mL 10% KF aqueous solution, stirred at room temperature for 2 hours. The suspension solution was filtered and the filter cake was washed with DCM (20 mL×2) and MeOH (20 mL), dried in vacuo to give 9-acetyl-2-(isoindolin-2-yl)-3,7-dimethyl-4H-pyrido[1,2-a]pyrimidin-4-one (3.5 g, crude) as a yellow solid that required no further purification. ¹H NMR (400 MHz, CDCl₃) δ 8.92 (s, 1H), 7.86 (s, 1H), 7.36-7.30 (m, 4H), 5.17 (s, 4H), 2.92 (s, 3H), 2.52 (s, 3H), 2.39 (s, 3H). MS: m/z 334.0 (M+H⁺).

### Step 3—Synthesis of 9-(1-hydroxyethyl)-2-(isoindolin-2-yl)-3,7-dimethyl-4H-pyrido[1,2-a]pyrimidin-4-one

To a solution of 9-acetyl-2-isoindolin-2-yl-3,7-dimethyl-pyrido[1,2-a]pyrimidin-4-one (3.5 g, 10.5 mmol) in MeOH (40 mL) was added NaBH₄ (1.2 g, 31.5 mmol) at 0° C. The mixture was stirred at room temperature for 16 hours under N₂ atmosphere. The reaction was quenched with 1M HCl (10 mL) at 0° C. Then the suspension solution was filtered and the filter cake was washed with EtOAc (20 mL×2) to give 9-(1-hydroxyethyl)-2-(isoindolin-2-yl)-3,7-dimethyl-4H-pyrido[1,2-a]pyrimidin-4-one (3.5 g, crude) as a yellow solid that required no further purification. ¹H NMR (400 MHz, CDCl₃) δ 8.72 (s, 1H), 7.40 (s, 1H), 7.31-7.37 (m, 4H), 5.20-5.25 (m, 1H), 5.12-5.19 (m, 4H), 2.52 (s, 3H), 2.37 (s, 3H), 1.70 (d, J=6.8 Hz, 3H). MS: m/z 336.1 (M+H⁺).

### Step 4—Synthesis of 9-(1-bromoethyl)-2-(isoindolin-2-yl)-3,7-dimethyl-4H-pyrido[1,2-a]pyrimidin-4-one

To a solution of 9-(1-hydroxyethyl)-2-(isoindolin-2-yl)-3,7-dimethyl-4H-pyrido[1,2-a]pyrimidin-4-one (1 g, 2.98 mmol) in DCM (20 mL) was added PBr₃ (847 mg, 3.13 mmol) at 0° C. The mixture was stirred at room temperature for 1 hour under N₂ atmosphere. The mixture was quenched with sat. aq. NaHCO₃ (20 mL), extracted with DCM (50 mL×2). The combined organic layers were washed with brine (20 mL), dried over anhydrous Na₂SO₄, filtered and concentrated in vacuo to give 9-(1-bromoethyl)-2-(isoindolin-2-yl)-3,7-dimethyl-4H-pyrido[1,2-a]pyrimidin-4-one (1.2 g, crude) as a yellow solid that required no further purification. ¹H NMR (400 MHz, CDCl₃) δ 8.83 (s, 1H), 7.95 (s, 1H), 7.43-7.37 (m, 4H), 5.89-6.08 (m, 1H), 5.33-5.45 (m, 4H), 2.90 (s, 3H), 2.52 (s, 3H), 2.17 (d, J=6.4 Hz, 3H).

### Step 5—Synthesis of 9-(1-((2-bromophenyl)amino)ethyl)-2-(isoindolin-2-yl)-3,7-dimethyl-4H-pyrido[1,2-a]pyrimidin-4-one

A mixture of 9-(1-bromoethyl)-2-(isoindolin-2-yl)-3,7-dimethyl-4H-pyrido[1,2-a]pyrimidin-4-one (100 mg, 251 umol) and 2-bromoaniline (86 mg, 502 umol) in dioxane (1 mL) was stirred at 90° C. for 2 hours. After cooling to room temperature, the mixture was concentrated in vacuo. The crude residue was purified by silica gel chromatography (solvent gradient: 0-50% EtOAc in petroleum ether) to give 9-(1-((2-bromophenyl)amino)ethyl)-2-(isoindolin-2-yl)-3,7-dimethyl-4H-pyrido[1,2-a]pyrimidin-4-one (80 mg, 80%) as a yellow solid. MS: m/z 491.2 (M+H⁺).

### Step 6—Synthesis of 2-(isoindolin-2-yl)-3,7-dimethyl-9-(1-((2-(pyridazin-4-yl)phenyl)amino) ethyl)-4H-pyrido[1,2-a]pyrimidin-4-one

To a solution of 9-(1-((2-bromophenyl)amino)ethyl)-2-(isoindolin-2-yl)-3,7-dimethyl-4H-pyrido[1,2-a]pyrimidin-4-one (130 mg, 266 μmol) in dioxane (2 mL) was added Pd(dppf)Cl₂ (19 mg, 27 μmol), Na₂CO₃ (84 mg, 797 μmol) and tributyl(pyridazin-4-yl)stannane (103 mg, 279 mol). The mixture was stirred at 100° C. for 16 hours under N₂ atmosphere. After cooling to room temperature, the mixture was concentrated in vacuo. The residue was purified by reverse phase chromatography (acetonitrile 62-92%/0.05% NH₃·H₂O+10 mM NH₄HCO₃ in water) to give 2-(isoindolin-2-yl)-3,7-dimethyl-9-(1-((2-(pyridazin-4-yl)phenyl)amino) ethyl)-4H-pyrido[1,2-a]pyrimidin-4-one (60 mg, 46%) as a white solid. ¹H NMR (400 MHz, DMSO-d₆) δ 9.42 (s, 1H), 9.24 (d, J=4.8 Hz, 1H), 8.55 (s, 1H), 7.88 (dd, J=5.2, 2.4 Hz, 1H), 7.72 (d, J=2.0 Hz, 1H), 7.38-7.43 (m, 2H), 7.29-7.34 (m, 2H), 7.09-7.15 (m, 2H), 6.73-6.68 (m, 1H), 6.45 (d, J=8.8 Hz, 1H), 5.84 (d, J=7.2 Hz, 1H), 5.29-5.25 (m, 1H), 5.19-5.12 (m, 2H), 5.01-5.09 (m, 2H), 2.42 (s, 3H), 2.27 (s, 3H), 1.51 (d, J=6.8 Hz, 3H). MS: m/z 489.2 (M+H⁺).

### Step 7—Synthesis of (R)-2-(isoindolin-2-yl)-3,7-dimethyl-9-(1-((2-(pyridazin-4-yl)phenyl)amino)ethyl)-4H-pyrido[1,2-a]pyrimidin-4-one & (S)-2-(isoindolin-2-yl)-3,7-dimethyl-9-(1-((2-(pyridazin-4-yl)phenyl)amino)ethyl)-4H-pyrido[1,2-a]pyrimidin-4-one (Compound 909)

2-(isoindolin-2-yl)-3,7-dimethyl-9-(1-((2-(pyridazin-4-yl)phenyl)amino)ethyl)-4H-pyrido[1,2-a]pyrimidin-4-one (50 mg, 102 umol) was separated by using chiral SFC (DAICEL CHIRALCEL OD (250 mm*30 mm, 10 um), Supercritical CO₂/EtOH+0.1% NH₄OH=55/45; 100 mL/min) to give (R)-2-(isoindolin-2-yl)-3,7-dimethyl-9-(1-((2-(pyridazin-4-yl)phenyl)amino)ethyl)-4H-pyrido[1,2-a]pyrimidin-4-one (22 mg, first peak) and (S)-2-(isoindolin-2-yl)-3,7-dimethyl-9-(1-((2-(pyridazin-4-yl)phenyl)amino)ethyl)-4H-pyrido[1,2-a]pyrimidin-4-one (23 mg, second peak) both as white solid. Absolute configuration was arbitrarily assigned to each enantiomer. Compound 909: ¹H NMR (400 MHz, DMSO-d₆) δ 9.41 (s, 1H), 9.24 (d, J=5.6 Hz, 1H), 8.55 (s, 1H), 7.87 (dd, J=5.2, 2.4 Hz, 1H), 7.72 (d, J=1.2 Hz, 1H), 7.42-7.36 (m, 2H), 7.33-7.28 (m, 2H), 7.08-7.13 (m, 2H), 6.73-6.69 (m, 1H), 6.45 (d, J=8.4 Hz, 1H), 5.83 (d, J=7.2 Hz, 1H), 5.29-5.23 (m, 1H), 5.11-5.19 (m, 2H), 4.99-5.09 (m, 2H), 2.42 (s, 3H), 2.26 (s, 3H), 1.50 (d, J=6.8 Hz, 3H). MS: m/z 489.2 (M+H⁺).

### Example 29: Preparation of (R)-2-((1-(2-(isoindolin-2-yl)-3,7-dimethyl-4-oxo-4H-pyrido[1,2-a]pyrimidin-9-yl)ethyl)amino)benzenesulfonamide (Compound 856)

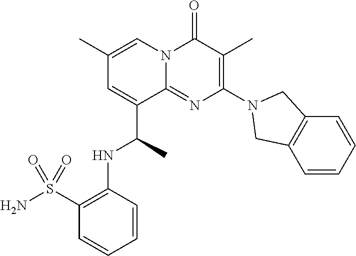 [SMILES: Cc1cc([C@@H](C)Nc2ccccc2S(N)(=O)=O)c2nc(N3Cc4ccccc4C3)c(C)c(=O)n2c1]

### Step 1—Synthesis of 2-((1-(2-(isoindolin-2-yl)-3,7-dimethyl-4-oxo-4H-pyrido[1,2-a]pyrimidin-9-yl)ethyl)amino)benzenesulfonamide

A mixture of 9-(1-bromoethyl)-2-(isoindolin-2-yl)-3,7-dimethyl-4H-pyrido[1,2-a]pyrimidin-4-one (500 mg, 1.26 mmol) and 2-aminobenzenesulfonamide (432 mg, 2.51 mmol) in dioxane (5 mL) was stirred at 90° C. for 2 hours. After cooling to room temperature, the reaction was concentrated in vacuo. The residue was purified by reverse phase chromatography (acetonitrile 58-88%/0.225% formic acid in water) to give 2-((1-(2-(isoindolin-2-yl)-3,7-dimethyl-4-oxo-4H-pyrido[1,2-a]pyrimidin-9-yl)ethyl)amino)benzenesulfonamide (100 mg, 16%) as a white solid. ¹H NMR (400 MHz, DMSO-d₆) δ 8.57 (s, 1H), 7.70 (d, J=2.0 Hz, 1H), 7.65 (dd, J=8.0, 1.6 Hz, 1H), 7.54 (s, 2H), 7.41 (dd, J=5.6, 3.2 Hz, 2H), 7.34-7.29 (m, 2H), 7.23-7.19 (m, 1H), 6.67-6.61 (m, 1H), 6.43 (d, J=5.6 Hz, 1H), 6.39 (d, J=8.4 Hz, 1H), 5.42-5.36 (m, 1H), 5.14-5.25 (m, 4H), 2.44 (s, 3H), 2.25 (s, 3H), 1.63 (d, J=6.8 Hz, 3H). MS: m/z 490.2 (M+H⁺).

### Step 2—Synthesis of (R)-2-((1-(2-(isoindolin-2-yl)-3,7-dimethyl-4-oxo-4H-pyrido[1,2-a]pyrimidin-9-yl)ethyl)amino)benzenesulfonamide (Compound 39) & (S)-2-((1-(2-(isoindolin-2-yl)-3,7-dimethyl-4-oxo-4H-pyrido[1,2-a]pyrimidin-9-yl)ethyl)amino)benzenesulfonamide

2-((1-(2-(isoindolin-2-yl)-3,7-dimethyl-4-oxo-4H-pyrido[1,2-a]pyrimidin-9-yl)ethyl)amino) benzenesulfonamide (90 mg, 184 umol) was separated by chiral SFC (DAICEL CHIRALCEL Cellulose-4 (250 mm*30 mm, 5 um), Supercritical CO₂/MeOH+0.1% NH₃·H₂O=40/60; 80 mL/min) to give (R)-2-((1-(2-(isoindolin-2-yl)-3,7-dimethyl-4-oxo-4H-pyrido[1,2-a]pyrimidin-9-yl)ethyl)amino)benzenesulfonamide (35 mg, first peak) and (S)-2-((1-(2-(isoindolin-2-yl)-3,7-dimethyl-4-oxo-4H-pyrido[1,2-a]pyrimidin-9-yl)ethyl)amino)benzenesulfonamide (29 mg, second peak) both as white solid. Absolute configuration was arbitrarily assigned to each enantiomer. Compound 919: ¹H NMR (400 MHz, DMSO-d₆) δ 8.56 (s, 1H), 7.70 (d, J=1.6 Hz, 1H), 7.64 (d, J=7.6 Hz, 1H), 7.55 (s, 2H), 7.43-7.37 (m, 2H), 7.33-7.26 (m, 2H), 7.22-7.18 (m, 1H), 6.66-6.62 (m, 1H), 6.43 (d, J=5.6 Hz, 1H), 6.38 (d, J=8.4 Hz, 1H), 5.41-5.35 (m, 1H), 5.26-5.11 (m, 4H), 2.43 (s, 3H), 2.24 (s, 3H), 1.62 (d, J=6.4 Hz, 3H). MS: m/z 490.1 (M+H⁺).

### Example 30: Preparation of 2-(isoindolin-2-yl)-3,7-dimethyl-9-(1-((2-(pyridazin-4-yl)thiophen-3-yl)amino)ethyl)-4H-pyrido[1,2-a]pyrimidin-4-one (Compound 1409)

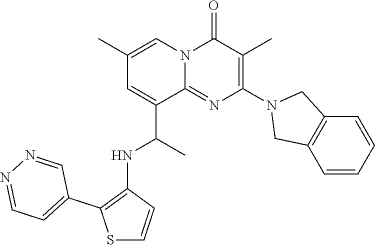 [SMILES: Cc1cc(C(C)Nc2ccsc2-c2ccnnc2)c2nc(N3Cc4ccccc4C3)c(C)c(=O)n2c1]

### Step 1—Synthesis of 9-(1-((2-bromothiophen-3-yl)amino)ethyl)-2-(isoindolin-2-yl)-3,7-dimethyl-4H-pyrido[1,2-a]pyrimidin-4-one

To a solution of 9-(1-bromoethyl)-2-(isoindolin-2-yl)-3,7-dimethyl-4H-pyrido[1,2-a]pyrimidin-4-one (20 mg, 50 μmol) in DMF (2 mL) was added 2-bromothiophen-3-amine hydrobromide (15.6 mg, 60 μmol) (prepared according to the procedure in WO2016144848) and DIEA (26 uL, 150 μmol). The mixture was stirred at 90° C. for 16 hours. After cooling to room temperature, the mixture was concentrated in vacuo. The resulting residue was purified by Prep-TLC (50% EtOAc in petroleum ether) to give 9-(1-((2-bromothiophen-3-yl)amino)ethyl)-2-(isoindolin-2-yl)-3,7-dimethyl-4H-pyrido[1,2-a]pyrimidin-4-one (15 mg, 55%) as a yellow solid. MS: m/z 495.0 (M+H⁺).

### Step 2—Synthesis of 2-(isoindolin-2-yl)-3,7-dimethyl-9-(1-((2-(pyridazin-4-yl)thiophen-3-yl)amino)ethyl)-4H-pyrido[1,2-a]pyrimidin-4-one

To a solution of 9-(1-((2-bromothiophen-3-yl)amino)ethyl)-2-(isoindolin-2-yl)-3,7-dimethyl-4H-pyrido[1,2-a]pyrimidin-4-one (20 mg, 40 mol) in dioxane (2 mL) was added Pd(PPh₃)₂Cl₂ (3 mg, 4 mol), Na₂CO₃ (13 mg, 120 μmol) and tributyl(pyridazin-4-yl)stannane (30 mg, 81 mol). The mixture was stirred at 100° C. for 16 hours under N₂ atmosphere. After cooling to room temperature, the mixture was concentrated in vacuo. The residue was purified by reverse phase chromatography (acetonitrile 46-76%/0.225% formic acid in water) to give 2-(isoindolin-2-yl)-3,7-dimethyl-9-(1-((2-(pyridazin-4-yl)thiophen-3-yl)amino)ethyl)-4H-pyrido[1,2-a]pyrimidin-4-one (Compound 963) (2.5 mg, 12%) as a yellow solid. ¹H NMR (400 MHz, DMSO-d₆) δ 9.47 (d, J=1.6 Hz, 1H), 9.08 (d, J=6.0 Hz, 1H), 8.56 (s, 1H), 7.81 (s, 1H), 7.76-7.74 (m, 1H), 7.54 (d, J=5.2 Hz, 1H), 7.41-7.47 (m, 1H), 7.32-7.30 (m, 2H), 6.53-6.61 (m, 2H), 5.30-5.43 (m, 1H), 5.04-5.21 (m, 4H), 2.42 (s, 3H), 2.28 (s, 3H), 1.58 (d, J=6.8 Hz, 3H). MS: m/z 495.2 (M+H⁺).

### Example 31: Preparation of (R)-2-((1-(2-(1,3-dimethyl-1H-indol-5-yl)-3,7-dimethyl-4-oxo-4H-pyrido[1,2-a]pyrimidin-9-yl)ethyl)amino)benzoic acid (Compound 902)

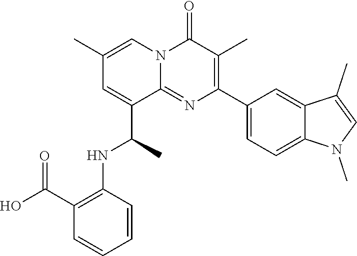 [SMILES: Cc1cc([C@@H](C)Nc2ccccc2C(=O)O)c2nc(-c3ccc4c(c3)c(C)cn4C)c(C)c(=O)n2c1]

### Step 1—Synthesis of 2-((1-(2-(1,3-dimethyl-1H-indol-5-yl)-3,7-dimethyl-4-oxo-4H-pyrido[1,2-a]pyrimidin-9-yl)ethyl)amino)benzoate

To a solution of tert-butyl 2-((1-(3,7-dimethyl-4-oxo-2-(((trifluoromethyl)sulfonyl)oxy)-4H-pyrido[1,2-a]pyrimidin-9-yl)ethyl)amino)benzoate (200 mg, 369 μmol), K₂CO₃ (153 mg, 1.11 mmol) and 1,3-dimethyl-5-(4,4,5,5-tetramethyl-1,3,2-dioxaborolan-2-yl)indole (100 mg, 369 mol) (prepared according to the procedure in WO201910295) in dioxane (5 mL) and water (1 mL) was added Pd(PPh₃)₄ (43 mg, 37 μmol). The reaction was stirred at 100° C. for 2 hours under N₂ atmosphere. After cooling to room temperature, the reaction was filtered and concentrated in vacuo. The crude residue was purified by silica gel chromatography (solvent gradient: 0-15% EtOAc in petroleum) to give 2-((1-(2-(1,3-dimethyl-1H-indol-5-yl)-3,7-dimethyl-4-oxo-4H-pyrido[1,2-a]pyrimidin-9-yl)ethyl)amino)benzoate (150 mg, 76%) as a yellow solid. MS: m/z 537.2 (M+H⁺).

### Step 2—Synthesis of 2-((1-(2-(1,3-dimethyl-1H-indol-5-yl)-3,7-dimethyl-4-oxo-4H-pyrido[1,2-a]pyrimidin-9-yl)ethyl)amino)benzoic acid

To a mixture of 2-((1-(2-(1,3-dimethyl-1H-indol-5-yl)-3,7-dimethyl-4-oxo-4H-pyrido[1,2-a]pyrimidin-9-yl)ethyl)amino)benzoate (150 mg, 280 μmol) in DCM (4 mL) was added TFA (2 mL). The reaction mixture was stirred at room temperature for 16 hours. The mixture was concentrated in vacuo. The residue was purified by reverse phase chromatography (acetonitrile 63%-93%/0.225% formic acid in water) to give 2-((1-(2-(1,3-dimethyl-1H-indol-5-yl)-3,7-dimethyl-4-oxo-4H-pyrido[1,2-a]pyrimidin-9-yl)ethyl)amino)benzoic acid (90 mg, 67%) as a white solid. ¹H NMR (400 MHz, DMSO-d₆) δ 12.77 (s, 1H), 8.69 (s, 1H), 8.43 (d, J=6.0 Hz, 1H), 7.88 (s, 1H), 7.83-7.78 (m, 1H), 7.66-7.62 (m, 1H), 7.63 (s, 1H), 7.60-7.54 (m, 1H), 7.50 (d, J=8.4 Hz, 1H), 7.23-7.14 (m, 2H), 6.54 (t, J=7.4 Hz, 1H), 6.39 (d, J=8.4 Hz, 1H), 5.47-5.42 (m, 1H), 3.77 (s, 3H), 2.34-2.24 (m, 9H), 2.07 (s, 1H), 1.62 (d, J=6.4 Hz, 3H). MS: m/z 481.2 (M+H⁺).

### Step 3—Synthesis of (R)-2-((1-(2-(1,3-dimethyl-1H-indol-5-yl)-3,7-dimethyl-4-oxo-4H-pyrido[1,2-a]pyrimidin-9-yl)ethyl)amino)benzoic acid (Compound 983) & (S)-2-((1-(2-(1,3-dimethyl-1H-indol-5-yl)-3,7-dimethyl-4-oxo-4H-pyrido[1,2-a]pyrimidin-9-yl)ethyl)amino)benzoic acid

2-((1-(2-(1,3-dimethyl-1H-indol-5-yl)-3,7-dimethyl-4-oxo-4H-pyrido[1,2-a]pyrimidin-9-yl)ethyl)amino)benzoic acid (90 mg, 187 mol) was separated by using chiral SFC (DAICEL CHIRALCEL OJ (250 mm*30 mm, 10 um); Supercritical CO₂/EtOH+0.1% NH₃·H₂O=65/35; 80 mL/min) to afford (R)-2-((1-(2-(1,3-dimethyl-1H-indol-5-yl)-3,7-dimethyl-4-oxo-4H-pyrido[1,2-a]pyrimidin-9-yl)ethyl)amino)benzoic acid (25 mg, first peak) and (S)-2-((1-(2-(1,3-dimethyl-1H-indol-5-yl)-3,7-dimethyl-4-oxo-4H-pyrido[1,2-a]pyrimidin-9-yl)ethyl)amino)benzoic acid (31 mg, second peak) both as white solid. Absolute configuration was arbitrarily assigned to each enantiomer. Compound 983: ¹H NMR (400 MHz, DMSO-d₆) δ 8.70 (s, 1H), 8.47 (s, 1H), 7.89 (s, 1H), 7.82 (d, J=7.2 Hz, 1H), 7.64 (s, 1H), 7.58 (d, J=7.6 Hz, 1H), 7.51 (d, J=8.4 Hz, 1H), 7.23-7.17 (m, 2H), 6.54 (t, J=7.8 Hz, 1H), 6.39 (d, J=8.4 Hz, 1H), 5.46 (s, 1H), 3.78 (s, 3H), 2.35-2.28 (m, 9H), 1.62 (d, J=6.4 Hz, 3H). MS: m/z 481.1 (M+H⁺).

### Example 32: Preparation of (R)-2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoic acid (Compound 892)

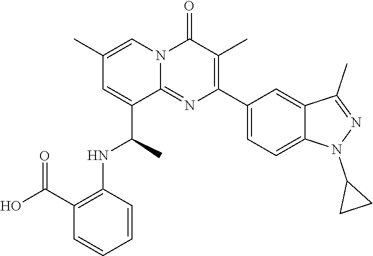 [SMILES: Cc1cc([C@@H](C)Nc2ccccc2C(=O)O)c2nc(-c3ccc4c(c3)c(C)nn4C3CC3)c(C)c(=O)n2c1]

### Step 1—Synthesis of tert-butyl 2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoate

To a solution of tert-butyl 2-((1-(3,7-dimethyl-4-oxo-2-(((trifluoromethyl)sulfonyl)oxy)-4H-pyrido[1,2-a]pyrimidin-9-yl)ethyl)amino)benzoate (200 mg, 369 μmol), K₂CO₃ (153 mg, 1.11 mmol) and 1-cyclopropyl-3-methyl-5-(4,4,5,5-tetramethyl-1,3,2-dioxaborolan-2-yl)-1H-indazole (165 mg, 554 mol) (prepared according to the procedure of 1-cyclopropyl-3-methyl-6-(4,4,5,5-tetramethyl-1,3,2-dioxaborolan-2-yl)-1H-indazole in WO201657834) in dioxane (2 mL) and water (0.2 mL) was added Pd(PPh₃)₄ (43 mg, 37 μmol). The reaction was stirred at 80° C. for 16 hours under N₂ atmosphere. After cooling to room temperature, the reaction was filtered and concentrated in vacuo. The crude residue was purified by silica gel chromatography (solvent gradient: 0-25% EtOAc in petroleum) to give tert-butyl 2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoate (124 mg, 60%) as a white solid. ¹H NMR (400 MHz, DMSO-d₆) δ 8.71 (s, 1H), 8.26 (d, J=6.0 Hz, 1H), 8.04 (s, 1H), 7.85-7.80 (m, 1H), 7.70 (s, 1H), 7.24-7.18 (m, 1H), 6.54 (t, J=7.6 Hz, 1H), 6.44 (d, J=8.4 Hz, 1H), 5.43-5.37 (m, 1H), 3.72-3.65 (m, 1H), 2.35 (s, 3H), 2.30 (s, 3H), 1.64 (d, J=6.8 Hz, 3H), 1.52 (s, 9H), 1.09-1.13 (m, 4H). MS: m/z 564.3 (M+H⁺).

### Step 2—Synthesis of 2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoic acid

To a mixture of tert-butyl 2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoate (124 mg, 220 mol) in DCM (2 mL) was added TFA (1 mL). The reaction mixture was stirred at room temperature for 16 hours. The mixture was concentrated in vacuo. The residue was purified by reverse phase chromatography (acetonitrile 55%-85%/0.225% formic acid in water) to give 2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoic acid (62 mg, 55%) as a white solid. ¹H NMR (400 MHz, DMSO-d₆) δ 12.75 (s, 1H), 8.71 (s, 1H), 8.42 (d, J=6.0 Hz, 1H), 8.05 (s, 1H), 7.84-7.79 (m, 2H), 7.75-7.72 (m, 1H), 7.66 (d, J=1.6 Hz, 1H), 7.24-7.15 (m, 1H), 6.56-6.52 (m, 1H), 6.38 (d, J=8.4 Hz, 1H), 5.50-5.35 (m, 1H), 3.75-3.64 (m, 1H), 2.52 (s, 3H), 2.33 (s, 3H), 2.29 (s, 3H), 1.61 (d, J=6.8 Hz, 3H), 1.17-1.06 (m, 4H). MS: m/z 508.3 (M+H⁺).

### Step 3—Synthesis of (R)-2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoic acid (Compound 969) & (S)-2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoic acid

2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoic acid (62 mg, 122 mol) was separated by using chiral SFC (DAICEL CHIRALPAK AD (250 mm*30 mm, 10 um); Supercritical CO₂/EtOH+0.1% NH₃·H₂O=70/30; 80 mL/min) to afford (R)-2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoic acid (22 mg, first peak) and (S)-2-((1-(2-(1-cyclopropyl-3-methyl-1H-indazol-5-yl)-3,7-dimethyl-4-oxo-4H-pyrido[1,2-a]pyrimidin-9-yl)ethyl)amino)benzoic acid (20 mg, second peak) both as white solid. Absolute configuration was arbitrarily assigned to each enantiomer. Compound 969: ¹H NMR (400 MHz, DMSO-d₆) δ 8.71 (s, 1H), 8.50-8.40 (m, 1H), 8.05 (s, 1H), 7.84-7.79 (m, 2H), 7.76-7.72 (m, 1H), 7.66 (d, J=1.6 Hz, 1H), 7.23-7.15 (m, 1H), 6.56-6.52 (m, 1H), 6.38 (d, J=8.4 Hz, 1H), 5.48-5.37 (m, 1H), 3.73-3.66 (m, 1H), 2.52 (s, 3H), 2.34 (s, 3H), 2.30 (s, 3H), 1.61 (d, J=6.8 Hz, 3H), 1.13-1.08 (m, 4H). MS: m/z 508.2 (M+H⁺).

### Example 33: Preparation of (R)-2-((1-(2-(3-cyanophenyl)-3,7-dimethyl-4-oxo-4H-pyrido[1,2-a]pyrimidin-9-yl)ethyl)amino)benzoic acid (Compound 967)

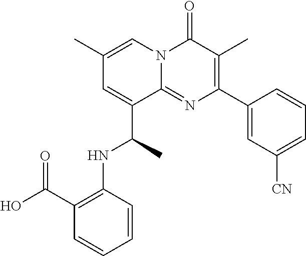 [SMILES: Cc1cc([C@@H](C)Nc2ccccc2C(=O)O)c2nc(-c3cccc(C#N)c3)c(C)c(=O)n2c1]

### Step 1—Synthesis of tert-butyl 2-((1-(2-(3-cyanophenyl)-3,7-dimethyl-4-oxo-4H-pyrido[1,2-a]pyrimidin-9-yl)ethyl)amino)benzoate

To a solution of tert-butyl 2-((1-(3,7-dimethyl-4-oxo-2-(((trifluoromethyl)sulfonyl)oxy)-4H-pyrido[1,2-a]pyrimidin-9-yl)ethyl)amino)benzoate (180 mg, 332 μmol), K₂CO₃ (138 mg, 997 umol) and (3-cyanophenyl)boronic acid (73 mg, 499 μmol) in dioxane (3 mL) and water (0.3 mL) was added Pd(PPh₃)₄ (38 mg, 33 mol). The reaction was stirred at 100° C. for 2 hours under N₂ atmosphere. After cooling to room temperature, the reaction was filtered and concentrated in vacuo. The crude residue was purified by silica gel chromatography (solvent gradient: 0-30% EtOAc in petroleum ether) to give tert-butyl 2-((1-(2-(3-cyanophenyl)-3,7-dimethyl-4-oxo-4H-pyrido[1,2-a]pyrimidin-9-yl)ethyl)amino)benzoate (120 mg, 73%) as a white solid. ¹H NMR (400 MHz, DMSO-d₆) δ 8.72 (s, 1H), 8.27-8.26 (m, 1H), 8.19 (s, 1H), 8.08 (d, J=8.0 Hz, 1H), 7.98 (d, J=7.6 Hz, 1H), 7.80-7.71 (m, 3H), 7.25-7.13 (m, 1H), 6.55-6.53 (m, 1H), 6.45 (d, J=8.8 Hz, 1H), 5.38-5.34 (m, 1H), 2.35 (s, 3H), 2.22 (s, 3H), 1.63 (d, J=6.8 Hz, 3H), 1.53 (s, 9H). MS: m/z 495.3 (M+H⁺).

### Step 2—Synthesis of 2-((1-(2-(3-cyanophenyl)-3,7-dimethyl-4-oxo-4H-pyrido[1,2-a]pyrimidin-9-yl)ethyl)amino)benzoic acid

To a mixture of tert-butyl 2-((1-(2-(3-cyanophenyl)-3,7-dimethyl-4-oxo-4H-pyrido[1,2-a]pyrimidin-9-yl)ethyl)amino)benzoate (60 mg, 121 μmol) in DCM (2 mL) was added TFA (0.5 mL, 6.9 mmol). The reaction mixture was stirred at room temperature for 16 hours. The mixture was concentrated in vacuo. The residue was purified by reverse phase chromatography (acetonitrile 58%-88%/0.225% formic acid in water) to give 2-((1-(2-(3-cyanophenyl)-3,7-dimethyl-4-oxo-4H-pyrido[1,2-a]pyrimidin-9-yl)ethyl)amino)benzoic acid (50 mg, 94%) as a white solid. ¹H NMR (400 MHz, DMSO-d₆) δ 12.75 (s, 1H), 8.72 (s, 1H), 8.44 (s, 1H), 8.19 (s, 1H), 8.08 (d, J=8.0 Hz, 1H), 7.98 (d, J=8.0 Hz, 1H), 7.85-7.73 (m, 2H), 7.68 (d, J=1.6 Hz, 1H), 7.21-7.19 (m 1H), 6.55-6.54 (m, 1H), 6.36 (d, J=8.0 Hz, 1H), 5.41-5.39 (m, 1H), 2.34 (s, 3H), 2.23 (s, 3H), 1.60 (d, J=6.8 Hz, 3H). MS: m/z 439.2 (M+H⁺).

### Step 3—Synthesis of (R)-2-((1-(2-(3-cyanophenyl)-3,7-dimethyl-4-oxo-4H-pyrido[1,2-a]pyrimidin-9-yl)ethyl)amino)benzoic acid (Compound 1071) & (S)-2-((1-(2-(3-cyanophenyl)-3,7-dimethyl-4-oxo-4H-pyrido[1,2-a]pyrimidin-9-yl)ethyl)amino)benzoic acid

2-((1-(2-(3-cyanophenyl)-3,7-dimethyl-4-oxo-4H-pyrido[1,2-a]pyrimidin-9-yl)ethyl)amino)benzoic acid (50 mg, 114 μmol) was separated by using chiral SFC (DAICEL CHIRALCEL OJ (250 mm*30 mm, 10 um); Supercritical CO₂/EtOH+0.1% NH₃·H₂O=75/25; 70 mL/min) to afford (R)-2-((1-(2-(3-cyanophenyl)-3,7-dimethyl-4-oxo-4H-pyrido[1,2-a]pyrimidin-9-yl)ethyl)amino)benzoic acid (Compound 1071) (4 mg, first peak) and (S)-2-((1-(2-(3-cyanophenyl)-3,7-dimethyl-4-oxo-4H-pyrido[1,2-a]pyrimidin-9-yl)ethyl)amino)benzoic acid (5 mg, second peak) both as white solid. Absolute configuration was arbitrarily assigned to each enantiomer. First peak: ¹H NMR (400 MHz, DMSO-d₆) δ 8.72 (s, 1H), 8.45 (s, 1H), 8.19 (s, 1H), 8.08 (d, J=8.0 Hz, 1H), 7.98 (d, J=8.0 Hz, 1H), 7.83-7.73 (m, 2H), 7.68 (d, J=1.6 Hz, 1H), 7.23-7.15 (m, 1H), 6.56-6.54 (m, 1H), 6.36 (d, J=8.4 Hz, 1H), 5.43-5.36 (m, 1H), 2.34 (s, 3H), 2.23 (s, 3H), 1.60 (d, J=6.8 Hz, 3H). MS: m/z 439.2 (M+H⁻).

### Example 34: Preparation of (R)-2-((1-(2-(3-cyano-4-fluorophenyl)-3,7-dimethyl-4-oxo-4H-pyrido[1,2-a]pyrimidin-9-yl)ethyl)amino)benzoic acid (Compound 947)

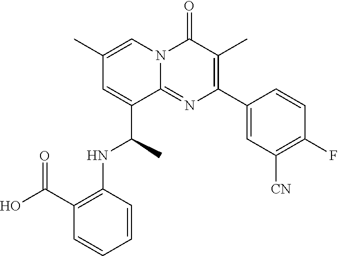 [SMILES: Cc1cc([C@@H](C)Nc2ccccc2C(=O)O)c2nc(-c3ccc(F)c(C#N)c3)c(C)c(=O)n2c1]

### Step 1—Synthesis of tert-butyl 2-((1-(2-(3-cyano-4-fluorophenyl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (3-cyano-4-fluoro-phenyl)boronic acid, tert-butyl 2-((1-(2-(3-cyano-4-fluorophenyl)-3,7-dimethyl-4-oxo-4H-pyrido[1,2-a]pyrimidin-9-yl)ethyl)amino)benzoate (160 mg, 84%) was obtained as a white solid. ¹H NMR (400 MHz, DMSO-d₆) δ 8.72 (s, 1H), 8.36-8.27 (m, 2H), 8.21-8.11 (m, 1H), 7.79-7.65 (m, 3H), 7.20-7.18 (m, 1H), 6.53-6.51 (m, 1H), 6.46 (d, J=8.4 Hz, 1H), 235 (s, 3H), 2.23 (s, 3H), 1.64 (d, J=6.4 Hz, 3H), 1.52 (s, 9H). MS: m/z 513.3 (M+H⁺).

### Step 2—Synthesis of 2-((1-(2-(3-cyano-4-fluorophenyl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(3-cyano-4-fluorophenyl)-3,7-dimethyl-4-oxo-4H-pyrido[1,2-a]pyrimidin-9-yl)ethyl)amino)benzoate, 2-((1-(2-(3-cyano-4-fluorophenyl)-3,7-dimethyl-4-oxo-4H-pyrido[1,2-a]pyrimidin-9-yl)ethyl)amino)benzoic acid (50 mg, 70%) was obtained as a white solid. ¹H NMR (400 MHz, DMSO-d₆) δ 12.74 (s, 1H), 8.72 (s, 1H), 8.52-8.35 (m, 1H), 8.30-8.29 (m, 1H), 8.20-8.12 (m, 1H), 7.80-7.79 (m, 1H), 7.73-7.68 (m, 2H), 7.23-7.15 (m, 1H), 6.55-6.54 (m, 1H), 6.36 (d, J 8.4 Hz, 1H), 5.44-5.35 (m, 1H), 2.34 (s, 3H), 2.23 (s, 3H), 1.60 (d, J=6.8 Hz, 3H). MS: m/z 457.2 (M+H⁺).

### Step 3—Synthesis of (R)-2-((1-(2-(3-cyano-4-fluorophenyl)-3,7-dimethyl-4-oxo-4H-pyrido[1,2-a]pyrimidin-9-yl)ethyl)amino)benzoic acid & (S)-2-((1-(2-(3-cyano-4-fluorophenyl)-3,7-dimethyl-4-oxo-4H-pyrido[1,2-a]pyrimidin-9-yl)ethyl)amino)benzoic acid

2-((1-(2-(3-cyano-4-fluorophenyl)-3,7-dimethyl-4-oxo-4H-pyrido[1,2-a]pyrimidin-9-yl)ethyl)amino)benzoic acid (50 mg, 110 mol) was separated by using chiral SFC (DAICEL CHIRALCEL OJ (250 mm*30 mm, 10 um); Supercritical CO₂/EtOH+0.1% NH₃·H₂O=65/35; 70 mL/min) to afford (R)-2-((1-(2-(3-cyano-4-fluorophenyl)-3,7-dimethyl-4-oxo-4H-pyrido[1,2-a]pyrimidin-9-yl)ethyl)amino)benzoic acid (Compound 1041) (9 mg, first peak) and (S)-2-((1-(2-(3-cyano-4-fluorophenyl)-3,7-dimethyl-4-oxo-4H-pyrido[1,2-a]pyrimidin-9-yl)ethyl)amino)benzoic acid (9 mg, second peak) both as white solid. Absolute configuration was arbitrarily assigned to each enantiomer. First peak: ¹H NMR (400 MHz, DMSO-d₆) δ 8.72 (s, 1H), 8.53-8.43 (m, 1H), 8.30-8.29 (m, 1H), 8.19-8.11 (m, 1H), 7.80-7.79 (m, 1H), 7.74-7.67 (m, 2H), 7.18-7.16 (m, 1H), 6.55-6.53 (m, 1H), 6.35 (d, J=8.4 Hz, 1H), 5.43-5.34 (m, 1H), 2.34 (s, 3H), 2.23 (s, 3H), 1.59 (d, J=6.4 Hz, 3H). MS: m/z 457.2 (M+H⁺).

### Example 35: Preparation of (R)-2-((1-(2-(4-chlorophenyl)-3,7-dimethyl-4-oxo-4H-pyrido[1,2-a]pyrimidin-9-yl)ethyl)amino)benzoic acid (Compound 963)

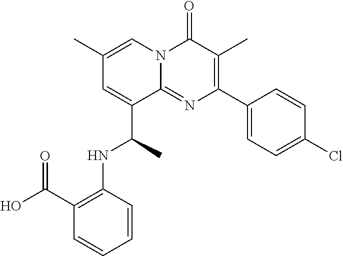 [SMILES: Cc1cc([C@@H](C)Nc2ccccc2C(=O)O)c2nc(-c3ccc(Cl)cc3)c(C)c(=O)n2c1]

### Step 1—Synthesis of tert-butyl 2-((1-(2-(4-chlorophenyl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (4-chlorophenyl)boronic acid, tert-butyl 2-((1-(2-(4-chlorophenyl)-3,7-dimethyl-4-oxo-4H-pyrido[1,2-a]pyrimidin-9-yl)ethyl)amino)benzoate (130 mg, 78%) was obtained as a white solid. MS: m/z 504.2 (M+H⁺).

### Step 2—Synthesis of 2-((1-(2-(4-chlorophenyl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(4-chlorophenyl)-3,7-dimethyl-4-oxo-4H-pyrido[1,2-a]pyrimidin-9-yl)ethyl)amino)benzoate, 2-((1-(2-(4-chlorophenyl)-3,7-dimethyl-4-oxo-4H-pyrido[1,2-a]pyrimidin-9-yl)ethyl)amino)benzoic acid (30 mg, 45%) was obtained as a white solid. MS: m/z 448.2 (M+H⁺).

### Step 3—Synthesis of (R)-2-((1-(2-(4-chlorophenyl)-3,7-dimethyl-4-oxo-4H-pyrido[1,2-a]pyrimidin-9-yl)ethyl)amino)benzoic acid (Compound 45) & (S)-2-((1-(2-(4-chlorophenyl)-3,7-dimethyl-4-oxo-4H-pyrido[1,2-a]pyrimidin-9-yl)ethyl)amino)benzoic acid

2-((1-(2-(4-chlorophenyl)-3,7-dimethyl-4-oxo-4H-pyrido[1,2-a]pyrimidin-9-yl)ethyl)amino)benzoic acid (20 mg, 45 umol) was separated by using chiral SFC (DAICEL CHIRALPAK AD (250 mm*30 mm, 10 um); Supercritical CO₂/EtOH+0.1% NH₃·H₂O=75/25; 70 mL/min) to afford (R)-2-((1-(2-(4-chlorophenyl)-3,7-dimethyl-4-oxo-4H-pyrido[1,2-a]pyrimidin-9-yl)ethyl)amino)benzoic acid (Compound 1067) (2 mg, first peak) and (S)-2-((1-(2-(4-chlorophenyl)-3,7-dimethyl-4-oxo-4H-pyrido[1,2-a]pyrimidin-9-yl)ethyl)amino)benzoic acid (3 mg, second peak) both as white solid. Absolute configuration was arbitrarily assigned to each enantiomer. First peak: ¹H NMR (400 MHz, DMSO-d₆) δ 12.76 (s, 1H), 8.70 (s, 1H), 8.47 (s, 1H), 7.85-7.72 (m, 3H), 7.68-7.67 (m, 1H), 7.63-7.60 (m, 2H), 7.24-7.11 (m, 1H), 6.58-6.50 (m, 1H), 6.35 (d, J=8.8 Hz, 1H), 5.42-5.34 (m, 1H), 2.34 (s, 3H), 2.24 (s, 3H), 1.60 (d, J=6.8 Hz, 3H). MS: m/z 448.1 (M+H⁺).

### Example 36: Preparation of (R)-2-((1-(2-(4-chloro-2-fluorophenyl)-3,7-dimethyl-4-oxo-4H-pyrido[1,2-a]pyrimidin-9-yl)ethyl)amino)benzoic acid (Compound 965)

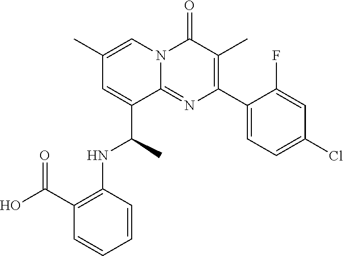 [SMILES: Cc1cc([C@@H](C)Nc2ccccc2C(=O)O)c2nc(-c3ccc(Cl)cc3F)c(C)c(=O)n2c1]

### Step 1—Synthesis of tert-butyl 2-((1-(2-(4-chloro-2-fluorophenyl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (4-chloro-2-fluoro-phenyl)boronic acid, tert-butyl 2-((1-(2-(4-chloro-2-fluorophenyl)-3,7-dimethyl-4-oxo-4H-pyrido[1,2-a]pyrimidin-9-yl)ethyl)amino)benzoate (135 mg, 78%) was obtained as a white solid. MS: m/z 522.2 (M+H⁺).

### Step 2—Synthesis of 2-((1-(2-(4-chloro-2-fluorophenyl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(4-chloro-2-fluorophenyl)-3,7-dimethyl-4-oxo-4H-pyrido[1,2-a]pyrimidin-9-yl)ethyl)amino)benzoate, 2-((1-(2-(4-chloro-2-fluorophenyl)-3,7-dimethyl-4-oxo-4H-pyrido[1,2-a]pyrimidin-9-yl)ethyl)amino)benzoic acid (30 mg, 45%) was obtained as a white solid. MS: m/z 466.2 (M+H⁺).

### Step 3—Synthesis of (R)-2-((1-(2-(4-chloro-2-fluorophenyl)-3,7-dimethyl-4-oxo-4H-pyrido[1,2-a]pyrimidin-9-yl)ethyl)amino)benzoic acid (Compound 1069) & (S)-2-((1-(2-(4-chloro-2-fluorophenyl)-3,7-dimethyl-4-oxo-4H-pyrido[1,2-a]pyrimidin-9-yl)ethyl)amino)benzoic acid

2-((1-(2-(4-chloro-2-fluorophenyl)-3,7-dimethyl-4-oxo-4H-pyrido[1,2-a]pyrimidin-9-yl)ethyl)amino)benzoic acid (30 mg, 65 umol) was separated by using chiral SFC (DAICEL CHIRALPAK AD (250 mm*30 mm, 10 um); Supercritical CO₂/EtOH+0.1% NH₃·H₂O=70/30; 60 mL/min) to afford (R)-2-((1-(2-(4-chloro-2-fluorophenyl)-3,7-dimethyl-4-oxo-4H-pyrido[1,2-a]pyrimidin-9-yl)ethyl)amino)benzoic acid (Compound 1069) (12 mg, first peak) and (S)-2-((1-(2-(4-chloro-2-fluorophenyl)-3,7-dimethyl-4-oxo-4H-pyrido[1,2-a]pyrimidin-9-yl)ethyl)amino)benzoic acid (7 mg, second peak) both as white solid. Absolute configuration was arbitrarily assigned to each enantiomer. First peak: ¹H NMR (400 M1 Hz, DMSO-d₆) δ 12.76 (s, 1H), 8.73 (s, 1H), 8.49 (s, 1H), 7.80 (d, J=8.0 Hz, 1H), 7.71-7.61 (m, 3H), 7.51-7.47 (m, 1H), 7.21-7.12 (m, 1H), 6.58-6.49 (m, 1H), 6.32 (d, J=8.8 Hz, 1H), 5.48-5.18 (m, 1H), 2.34 (s, 3H), 2.06 (s, 3H), 1.56 (d, J=6.4 Hz, 3H). MS: m/z 466.1 (M+H⁺).

### Example 37: Preparation of (R)-2-((1-(2-(6-cyanopyridin-3-yl)-3,7-dimethyl-4-oxo-4H-pyrido[1,2-a]pyrimidin-9-yl)ethyl)amino)benzoic acid (Compound 975)

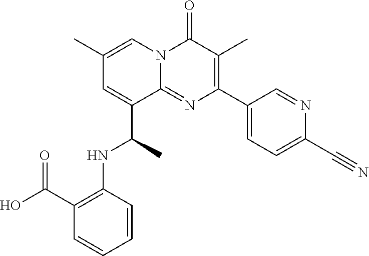 [SMILES: Cc1cc([C@@H](C)Nc2ccccc2C(=O)O)c2nc(-c3ccc(C#N)nc3)c(C)c(=O)n2c1]

### Step 1—Synthesis of tert-butyl 2-((1-(2-(6-cyanopyridin-3-yl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (6-cyano-3-pyridyl)boronic acid, tert-butyl 2-((1-(2-(6-cyanopyridin-3-yl)-3,7-dimethyl-4-oxo-4H-pyrido[1,2-a]pyrimidin-9-yl)ethyl)amino)benzoate (150 mg, 90%) was obtained as a white solid. MS: m/z 496.3 (M+H⁺).

### Step 2—Synthesis of 2-((1-(2-(6-cyanopyridin-3-yl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(6-cyanopyridin-3-yl)-3,7-dimethyl-4-oxo-4H-pyrido[1,2-a]pyrimidin-9-yl)ethyl)amino)benzoate, 2-((1-(2-(6-cyanopyridin-3-yl)-3,7-dimethyl-4-oxo-4H-pyrido[1,2-a]pyrimidin-9-yl)ethyl)amino)benzoic acid (30 mg, 17%) was obtained as a white solid. MS: m/z 440.2 (M+H⁺).

### Step 3—Synthesis of (R)-2-((1-(2-(6-cyanopyridin-3-yl)-3,7-dimethyl-4-oxo-4H-pyrido[1,2-a]pyrimidin-9-yl)ethyl)amino)benzoic acid (Compound 1081) & (S)-2-((1-(2-(6-cyanopyridin-3-yl)-3,7-dimethyl-4-oxo-4H-pyrido[1,2-a]pyrimidin-9-yl)ethyl)amino)benzoic acid

2-((1-(2-(6-cyanopyridin-3-yl)-3,7-dimethyl-4-oxo-4H-pyrido[1,2-a]pyrimidin-9-yl)ethyl)amino)benzoic acid (20 mg, 46 umol) was separated by using chiral SFC (DAICEL CHIRALPAK AD (250 mm*30 mm, 10 um); Supercritical CO₂/EtOH+0.1% NH₃·H₂O=70/30; 60 mL/min) to afford (R)-2-((1-(2-(6-cyanopyridin-3-yl)-3,7-dimethyl-4-oxo-4H-pyrido[1,2-a]pyrimidin-9-yl)ethyl)amino)benzoic acid (Compound 1081) (6 mg, first peak) and (S)-2-((1-(2-(6-cyanopyridin-3-yl)-3,7-dimethyl-4-oxo-4H-pyrido[1,2-a]pyrimidin-9-yl)ethyl)amino)benzoic acid (5 mg, second peak) both as white solid. Absolute configuration was arbitrarily assigned to each enantiomer. First peak: ¹H NMR (400 MHz, DMSO-d₆) δ 12.74 (s, 1H), 9.13 (d, J=1.6 Hz, 1H), 8.74 (s, 1H), 8.51-8.45 (m, 1H), 8.44-8.40 (m, 1H), 8.23 (d, J=8.0 Hz, 1H), 7.80 (d, J=7.2 Hz, 1H), 7.72 (s, 1H), 7.22-7.13 (m, 1H), 6.60-6.49 (m, 1H), 6.37 (d, J=8.8 Hz, 1H), 5.45-5.28 (m, 1H), 2.35 (s, 3H), 2.24 (s, 3H), 1.60 (d, J=6.4 Hz, 3H). MS: m/z 440.1 (M+H⁺).

### Example 38: Preparation of 2-((1-(3,7-dimethyl-4-oxo-2-(pyridin-4-yl)-4H-pyrido[1,2-a]pyrimidin-9-yl)ethyl)amino)benzoic acid (Compound 1408)

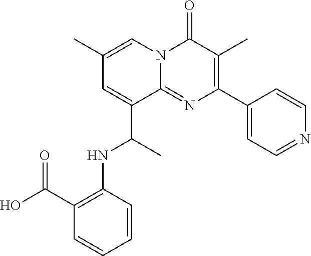 [SMILES: Cc1cc(C(C)Nc2ccccc2C(=O)O)c2nc(-c3ccncc3)c(C)c(=O)n2c1]

### Step 1—Synthesis of tert-butyl 2-((1-(3,7-dimethyl-4-oxo-2-(pyridin-4-yl)-4H-pyrido[1,2-a]pyrimidin-9-yl)ethyl)amino)benzoate

Following the procedure described in Example 33, Step 1 and making non-critical variations as required to replace (3-cyanophenyl)boronic acid with pyridin-4-ylboronic acid, tert-butyl 2-((1-(3,7-dimethyl-4-oxo-2-(pyridin-4-yl)-4H-pyrido[1,2-a]pyrimidin-9-yl)ethyl)amino)benzoate (130 mg, 83%) was obtained as a white solid. MS: m/z 471.3 (M+H⁺).

### Step 2—Synthesis of 2-((1-(3,7-dimethyl-4-oxo-2-(pyridin-4-yl)-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(3,7-dimethyl-4-oxo-2-(pyridin-4-yl)-4H-pyrido[1,2-a]pyrimidin-9-yl)ethyl)amino)benzoate, 2-((1-(3,7-dimethyl-4-oxo-2-(pyridin-4-yl)-4H-pyrido[1,2-a]pyrimidin-9-yl)ethyl)amino)benzoic acid (30 mg, 68%) was obtained as a white solid. ¹H NMR (400 MHz, DMSO-d₆) δ 12.77 (s, 1H), 8.78-8.74 (m, 2H), 8.72 (s, 1H), 8.41 (d, J=6.0 Hz, 1H), 7.83-7.78 (m, 1H), 7.76-7.72 (m, 2H), 7.70 (d, J=1.6 Hz, 1H), 7.23-7.15 (m, 1H), 6.58-6.50 (m, 1H), 6.36 (d, J=8.4 Hz, 1H), 5.44-5.31 (m, 1H), 2.34 (s, 3H), 2.23 (s, 3H), 1.60 (d, J=6.4 Hz, 3H). MS: m/z 415.1 (M+H⁺).

### Example 39: Preparation of (R)-2-((1-(2-(3-fluorophenyl)-3,7-dimethyl-4-oxo-4H-pyrido[1,2-a]pyrimidin-9-yl)ethyl)amino)benzoic acid (Compound 969)

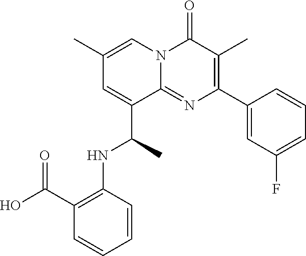 [SMILES: Cc1cc([C@@H](C)Nc2ccccc2C(=O)O)c2nc(-c3cccc(F)c3)c(C)c(=O)n2c1]

### Step 1—Synthesis of tert-butyl 2-((1-(2-(3-fluorophenyl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (3-fluorophenyl) boronic acid, tert-butyl 2-((1-(2-(3-fluorophenyl)-3,7-dimethyl-4-oxo-4H-pyrido[1,2-a]pyrimidin-9-yl)ethyl)amino)benzoate (120 mg, 67%) was obtained as a white solid. ¹H NMR (400 MHz, CDCl₃) δ 8.77 (s, 1H), 7.89 (dd, J=8.0, 1.6 Hz, 1H), 7.60 (d, J=2.0 Hz, 1H), 7.51-7.40 (m, 3H), 7.20-7.10 (m, 2H), 6.60-6.51 (m, 1H), 6.29 (d, J=8.4 Hz, 1H), 5.58-5.39 (m, 1H), 2.38 (s, 3H), 2.35 (s, 3H), 1.68 (d, J=6.8 Hz, 3H), 1.64 (s, 9H). MS: m/z 488.1 (M+H⁺).

### Step 2—Synthesis of 2-((1-(2-(3-fluorophenyl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(3-fluorophenyl)-3,7-dimethyl-4-oxo-4H-pyrido[1,2-a]pyrimidin-9-yl)ethyl)amino)benzoate, 2-((1-(2-(3-fluorophenyl)-3,7-dimethyl-4-oxo-4H-pyrido[1,2-a]pyrimidin-9-yl)ethyl)amino)benzoic acid (60 mg, 54%) was obtained as a yellow solid. ¹H NMR (400 MHz, DMSO-d₆) δ 12.78 (s, 1H), 8.71 (s, 1H), 8.41 (d, J=6.4 Hz, 1H), 7.86-7.75 (m, 1H), 7.66 (d, J=1.6 Hz, 1H), 7.63-7.52 (m, 3H), 7.39-7.30 (m, 1H), 7.24-7.13 (m, 1H), 6.51-6.55 (m, J=7.6 Hz, 1H), 6.35 (d, J=8.8 Hz, 1H), 5.42-5.36 (m, 1H), 2.33 (s, 3H), 2.23 (s, 3H), 1.59 (d, J=6.8 Hz, 3H). MS: m/z 432.2 (M+H⁺).

### Step 3—Synthesis of (R)-2-((1-(2-(3-fluorophenyl)-3,7-dimethyl-4-oxo-4H-pyrido[1,2-a]pyrimidin-9-yl)ethyl)amino)benzoic acid (Compound 1073) & (S)-2-((1-(2-(3-fluorophenyl)-3,7-dimethyl-4-oxo-4H-pyrido[1,2-a]pyrimidin-9-yl)ethyl)amino)benzoic acid

2-((1-(2-(3-fluorophenyl)-3,7-dimethyl-4-oxo-4H-pyrido[1,2-a]pyrimidin-9-yl)ethyl)amino)benzoic acid (60 mg, 139 mol) was separated by using chiral SFC (DAICEL CHIRALCEL OD (250 mm*30 mm, 10 um), Supercritical CO₂/EtOH+0.1% NH₃·H₂O=55/45; 100 mL/min) to afford (R)-2-((1-(2-(3-fluorophenyl)-3,7-dimethyl-4-oxo-4H-pyrido[1,2-a]pyrimidin-9-yl)ethyl)amino)benzoic acid (Compound 1073) (10 mg, first peak) and (S)-2-((1-(2-(3-fluorophenyl)-3,7-dimethyl-4-oxo-4H-pyrido[1,2-a]pyrimidin-9-yl)ethyl)amino)benzoic acid (13 mg, second peak) both as white solid. Absolute configuration was arbitrarily assigned to each enantiomer. First peak: ¹H NMR (400 MHz, DMSO-d₆) δ 8.71 (s, 1H), 8.45 (s, 1H), 7.81 (d, J=7.6 Hz, 1H), 7.67 (s, 1H), 7.60-7.53 (m, 3H), 7.37-7.31 (m, 1H), 7.21-7.16 (m, 1H), 6.55-6.51 (m, 1H), 6.35 (d, J=8.4 Hz, 1H), 5.41-5.36 (m, 1H), 2.33 (s, 3H), 2.23 (s, 3H), 1.60 (d, J=6.8 Hz, 3H). MS: m/z 432.2 (M+H⁺).

### Example 40: Preparation of (R)-2-((1-(2-(3-(difluoromethyl)phenyl)-3,7-dimethyl-4-oxo-4H-pyrido[1,2-a]pyrimidin-9-yl)ethyl)amino)benzoic acid (Compound 972)

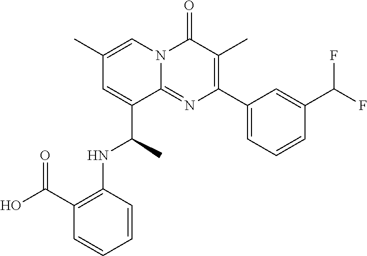 [SMILES: Cc1cc([C@@H](C)Nc2ccccc2C(=O)O)c2nc(-c3cccc(C(F)F)c3)c(C)c(=O)n2c1]

### Step 1—Synthesis of tert-butyl 2-((1-(2-(3-(difluoromethyl)phenyl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (3-(difluoromethyl)phenyl)boronic acid, tert-butyl 2-((1-(2-(3-(difluoromethyl)phenyl)-3,7-dimethyl-4-oxo-4H-pyrido[1,2-a]pyrimidin-9-yl)ethyl)amino)benzoate (160 mg, 83%) was obtained as a white solid. ¹H NMR (400 MHz, CDCl₃) δ 8.78 (s, 1H), 8.63-8.15 (m, 1H), 7.87-8.01 (m, 1H), 7.86-7.81 (m, 2H), 7.65-7.58 (m, 3H), 7.19-7.10 (m, 1H), 6.91-6.61 (m, 1H), 6.60-6.54 (m, 1H), 6.32 (d, J=8.4 Hz, 1H), 5.52-5.48 (m, 1H), 2.37 (s, 3H), 2.36 (s, 3H), 1.69 (d, J=6.8 Hz, 3H), 1.63 (s, 9H). MS: m/z 520.2 (M+H⁺).

### Step 2—Synthesis of 2-((1-(2-(3-(difluoromethyl)phenyl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(3-(difluoromethyl)phenyl)-3,7-dimethyl-4-oxo-4H-pyrido[1,2-a]pyrimidin-9-yl)ethyl)amino)benzoate, 2-((1-(2-(3-(difluoromethyl)phenyl)-3,7-dimethyl-4-oxo-4H-pyrido[1,2-a]pyrimidin-9-yl)ethyl)amino)benzoic acid (90 mg, 62%) was obtained as a white solid. ¹H NMR (400 MHz, DMSO-d₆) δ 12.75 (s, 1H), 8.72 (s, 1H), 8.42 (d, J=5.6 Hz, 1H), 7.96-7.88 (m, 2H), 7.81 (d, J=6.8 Hz, 1H), 7.73-7.65 (m, 3H), 7.31-7.01 (m, 2H), 6.56-6.52 (m, 1H), 6.36 (d, J=8.4 Hz, 1H), 5.42-5.36 (m, 1H), 2.34 (s, 3H), 2.23 (s, 3H), 1.60 (d, J=6.4 Hz, 3H). MS: m/z 464.2 (M+H⁺).

### Step 3—Synthesis of (R)-2-((1-(2-(3-(difluoromethyl)phenyl)-3,7-dimethyl-4-oxo-4H-pyrido[1,2-a]pyrimidin-9-yl)ethyl)amino)benzoic acid (Compound 1077) & (S)-2-((1-(2-(3-(difluoromethyl)phenyl)-3,7-dimethyl-4-oxo-4H-pyrido[1,2-a]pyrimidin-9-yl)ethyl)amino)benzoic acid

2-((1-(2-(3-(difluoromethyl)phenyl)-3,7-dimethyl-4-oxo-4H-pyrido[1,2-a]pyrimidin-9-yl)ethyl)amino)benzoic acid (90 mg, 194 mol) was separated by using chiral SFC (DAICEL CHIRALCEL OD (250 mm*30 mm, 10 um), Supercritical CO₂/EtOH+0.1% NH₃·H₂O=55/45; 100 mL/min) to give (R)-2-((1-(2-(3-(difluoromethyl)phenyl)-3,7-dimethyl-4-oxo-4H-pyrido[1,2-a]pyrimidin-9-yl)ethyl)amino)benzoic acid (Compound 1077) (22 mg, first peak) and (S)-2-((1-(2-(3-(difluoromethyl)phenyl)-3,7-dimethyl-4-oxo-4H-pyrido[1,2-a]pyrimidin-9-yl)ethyl)amino)benzoic acid (29 mg, second peak) both as white solid. Absolute configuration was arbitrarily assigned to each enantiomer. First peak: ¹H NMR (400 MHz, DMSO-d₆) δ 8.71 (s, 1H), 8.44 (s, 1H), 7.94-7.89 (m, 2H), 7.81 (d, J=7.6 Hz, 1H), 7.72-7.66 (m, 3H), 7.30-7.01 (m, 2H), 6.55-6.50 (m, 1H), 6.36 (d, J=8.4 Hz, 1H), 5.42-5.36 (m, 1H), 2.33 (s, 3H), 2.23 (s, 3H), 1.59 (d, J=6.4 Hz, 3H). MS: m/z 464.3 (M+H⁻).

### Example 41: Preparation of (R)-2-((1-(2-(5-chloro-6-methylpyridin-3-yl)-3,7-dimethyl-4-oxo-4H-pyrido[1,2-a]pyrimidin-9-yl)ethyl)amino)benzoic acid (Compound 951)

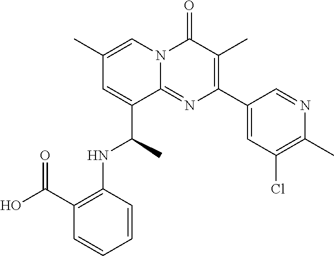 [SMILES: Cc1cc([C@@H](C)Nc2ccccc2C(=O)O)c2nc(-c3cnc(C)c(Cl)c3)c(C)c(=O)n2c1]

### Step 1—Synthesis of tert-butyl 2-((1-(2-(5-chloro-6-methylpyridin-3-yl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (5-chloro-6-methylpyridin-3-yl)boronic acid, tert-butyl 2-((1-(2-(5-chloro-6-methylpyridin-3-yl)-3,7-dimethyl-4-oxo-4H-pyrido[1,2-a]pyrimidin-9-yl)ethyl)amino)benzoate (170 mg, 89%) was obtained as a white solid. ¹H NMR (400 MHz, DMSO-d₆) δ 8.78 (d, J=2.0 Hz, 1H), 8.72 (s, 1H), 8.33 (d, J=6.8 Hz, 1H), 8.22 (d, J=2.0 Hz, 1H), 7.78-7.71 (m, 2H), 7.24-7.17 (m, 1H), 6.58-6.42 (m, 2H), 5.48-5.20 (m, 1H), 2.63 (s, 3H), 2.35 (s, 3H), 2.25 (s, 3H), 1.64 (d, J=6.8 Hz, 3H), 1.51 (s, 9H). MS: m/z 519.1 (M+H⁺).

### Step 2—Synthesis of 2-((1-(2-(5-chloro-6-methylpyridin-3-yl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(5-chloro-6-methylpyridin-3-yl)-3,7-dimethyl-4-oxo-4H-pyrido[1,2-a]pyrimidin-9-yl)ethyl)amino)benzoate, 2-((1-(2-(5-chloro-6-methylpyridin-3-yl)-3,7-dimethyl-4-oxo-4H-pyrido[1,2-a]pyrimidin-9-yl)ethyl)amino)benzoic acid (40 mg, 64%) was obtained as a white solid. ¹H NMR (400 MHz, DMSO-d₆) δ 12.76 (s, 1H), 8.80 (d, J=2.0 Hz, 1H), 8.72 (s, 1H), 8.44 (s, 1H), 8.22 (d, J=2.0 Hz, 1H), 7.83-7.78 (m, 1H), 7.69 (d, J=1.6 Hz, 1H), 7.22-7.16 (m, 1H), 6.57-6.51 (m, 1H), 6.37 (d, J=8.4 Hz, 1H), 5.43-5.33 (m, 1H), 2.64 (s, 3H), 2.34 (s, 3H), 2.25 (s, 3H), 1.60 (d, J=6.8 Hz, 3H). MS: m/z 463.2 (M+H⁺).

### Step 3—Synthesis of (R)-2-((1-(2-(5-chloro-6-methylpyridin-3-yl)-3,7-dimethyl-4-oxo-4H-pyrido[1,2-a]pyrimidin-9-yl)ethyl)amino)benzoic acid (Compound 1052) & (S)-2-((1-(2-(5-chloro-6-methylpyridin-3-yl)-3,7-dimethyl-4-oxo-4H-pyrido[1,2-a]pyrimidin-9-yl)ethyl)amino)benzoic acid

2-((1-(2-(5-chloro-6-methylpyridin-3-yl)-3,7-dimethyl-4-oxo-4H-pyrido[1,2-a]pyrimidin-9-yl)ethyl)amino)benzoic acid (40 mg, 86 umol) was separated by using chiral SFC (DAICEL CHIRALCEL AD (250 mm*30 mm, 10 um); Supercritical CO₂/EtOH+0.1% NH₃·H₂O=40/60; 60 mL/min) to afford (R)-2-((1-(2-(5-chloro-6-methylpyridin-3-yl)-3,7-dimethyl-4-oxo-4H-pyrido[1,2-a]pyrimidin-9-yl)ethyl)amino)benzoic acid (Compound 1052) (12 mg, first peak) and (S)-2-((1-(2-(5-chloro-6-methylpyridin-3-yl)-3,7-dimethyl-4-oxo-4H-pyrido[1,2-a]pyrimidin-9-yl)ethyl)amino)benzoic acid (11 mg, second peak) both as white solid. Absolute configuration was arbitrarily assigned to each enantiomer. First peak: ¹H NMR (400 MHz, DMSO-d₆) δ 12.87 (s, 1H), 8.80 (d, J=2.0 Hz, 1H), 8.72 (s, 1H), 8.49 (s, 1H), 8.22 (d, J=2.0 Hz, 1H), 7.82-7.78 (m, 1H), 7.69 (d, J=1.6 Hz, 1H), 7.20-7.15 (m, 1H), 6.56-6.50 (m, 1H), 6.36 (d, J=8.0 Hz, 1H), 5.42-5.33 (m, 1H), 2.64 (s, 3H), 2.34 (s, 3H), 2.26 (s, 3H), 1.60 (d, J=6.8 Hz, 3H). MS: m/z 463.0 (M+H⁺).

### Example 42: Preparation of (R)-2-((1-(2-(3-cyclopropylphenyl)-3,7-dimethyl-4-oxo-4H-pyrido[1,2-a]pyrimidin-9-yl)ethyl)amino)benzoic acid (Compound 971)

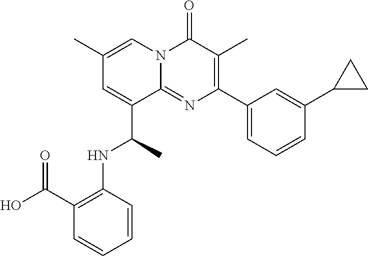 [SMILES: Cc1cc([C@@H](C)Nc2ccccc2C(=O)O)c2nc(-c3cccc(C4CC4)c3)c(C)c(=O)n2c1]

### Step 1—Synthesis of tert-butyl 2-((1-(2-(3-cyclopropylphenyl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (3-cyclopropylphenyl)boronic acid, tert-butyl 2-((1-(2-(3-cyclopropylphenyl)-3,7-dimethyl-4-oxo-4H-pyrido[1,2-a]pyrimidin-9-yl)ethyl)amino)benzoate (135 mg, 80%) was obtained as a white solid. ¹H NMR (400 MHz, DMSO-d₆) δ 8.70 (s, 1H), 8.21 (d, J=6.4 Hz, 1H), 7.76 (d, J=6.4 Hz, 1H), 7.68 (s, 1H), 7.50-7.44 (m, 1H), 7.43-7.36 (m, 2H), 7.17 (d, J=7.2 Hz, 2H), 6.56-6.54 (m, 1H), 6.40 (d, J=8.8 Hz, 1H), 5.45-5.35 (m, 1H), 2.71-2.62 (m, 1H), 2.34 (s, 3H), 2.22 (s, 3H), 1.62 (d, J=6.8 Hz, 3H), 1.56 (s, 9H), 1.02-0.93 (m, 2H), 0.76-0.69 (m, 2H). MS: m/z 510.2 (M+H⁺).

### Step 2—Synthesis of 2-((1-(2-(3-cyclopropylphenyl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(3-cyclopropylphenyl)-3,7-dimethyl-4-oxo-4H-pyrido[1,2-a]pyrimidin-9-yl)ethyl)amino)benzoate, 2-((1-(2-(3-cyclopropylphenyl)-3,7-dimethyl-4-oxo-4H-pyrido[1,2-a]pyrimidin-9-yl)ethyl)amino)benzoic acid (40 mg, 64%) was obtained as a white solid. ¹H NMR (400 MHz, DMSO-d₆) δ 12.77 (s, 1H), 8.70 (s, 1H), 8.41 (d, J=6.4 Hz, 1H), 7.84-7.75 (m, 1H), 7.65 (d, J=2.0 Hz, 1H), 7.51-7.45 (m, 1H), 7.43-7.37 (m, 2H), 7.23-7.15 (m, 2H), 6.57-6.50 (m, 1H), 6.36 (d, J=8.4 Hz, 1H), 5.45-5.35 (m, 1H), 2.33 (s, 3H), 2.22 (s, 3H), 2.06-1.97 (m, 1H), 1.60 (d, J=6.4 Hz, 3H), 1.02-0.95 (m, 2H), 0.75-0.70 (m, 2H). MS: m/z 454.1 (M+H⁺).

### Step 3—Synthesis of (R)-2-((1-(2-(3-cyclopropylphenyl)-3,7-dimethyl-4-oxo-4H-pyrido[1,2-a]pyrimidin-9-yl)ethyl)amino)benzoic acid (Compound 1075) & (S)-2-((1-(2-(3-cyclopropylphenyl)-3,7-dimethyl-4-oxo-4H-pyrido[1,2-a]pyrimidin-9-yl)ethyl)amino)benzoic acid

2-((1-(2-(3-cyclopropylphenyl)-3,7-dimethyl-4-oxo-4H-pyrido[1,2-a]pyrimidin-9-yl)ethyl)amino)benzoic acid (40 mg, 88 umol) was separated by using chiral SFC (DAICEL CHIRALCEL AD (250 mm*30 mm, 10 um); Supercritical CO₂/EtOH+0.1% NH₃·H₂O=20/80, 60 mL/min) to afford (R)-2-((1-(2-(3-cyclopropylphenyl)-3,7-dimethyl-4-oxo-4H-pyrido[1,2-a]pyrimidin-9-yl)ethyl)amino)benzoic acid (Compound 1075) (11 mg, first peak) and (S)-2-((1-(2-(3-cyclopropylphenyl)-3,7-dimethyl-4-oxo-4H-pyrido[1,2-a]pyrimidin-9-yl)ethyl)amino)benzoic acid (21 mg, second peak) both as white solid. Absolute configuration was arbitrarily assigned to each enantiomer. First peak: ¹H NMR (400 MHz, DMSO-d₆) δ 8.68 (s, 1H), 8.44 (s, 1H), 7.80 (d, J=8.0 Hz, 1H), 7.64 (s, 1H), 7.50-7.44 (m, 1H), 7.43-7.36 (m, 2H), 7.19-7.15 (m, 2H), 6.55-6.50 (m, 1H), 6.35 (d, J=8.4 Hz, 1H), 5.44-5.35 (m, 1H), 2.32 (s, 3H), 2.21 (s, 3H), 2.06-1.95 (m, 1H), 1.59 (d, J=6.4 Hz, 3H), 1.02-0.93 (m, 2H), 0.76-0.69 (m, 2H). MS: m/z 454.1 (M+H⁺).

### Example 43: Preparation of (R)-2-((1-(3,7-dimethyl-4-oxo-2-(pyridin-3-yl)-4H-pyrido[1,2-a]pyrimidin-9-yl)ethyl)amino)benzoic acid (Compound 949)

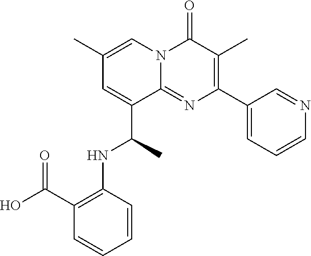 [SMILES: Cc1cc([C@@H](C)Nc2ccccc2C(=O)O)c2nc(-c3cccnc3)c(C)c(=O)n2c1]

### Step 1—Synthesis of tert-butyl 2-((1-(3,7-dimethyl-4-oxo-2-(pyridin-3-yl)-4H-pyrido[1,2-a]pyrimidin-9-yl)ethyl)amino)benzoate

Following the procedure described in Example 33, Step 1 and making non-critical variations as required to replace (3-cyanophenyl)boronic acid with pyridin-3-ylboronic acid, tert-butyl 2-((1-(3,7-dimethyl-4-oxo-2-(pyridin-3-yl)-4H-pyrido[1,2-a]pyrimidin-9-yl)ethyl)amino)benzoate (165 mg, 95%) was obtained as a white solid. ¹H NMR (400 MHz, DMSO-d₆) δ 8.95 (d, J=2.0 Hz, 1H), 8.72 (s, 1H), 8.70-8.68 (m, 1H), 8.27 (d, J=6.4 Hz, 1H), 8.18-8.15 (m, 1H), 7.78-7.70 (m, 2H), 7.59-7.56 (m, 1H), 7.23-7.16 (m, 1H), 6.55-6.51 (m, 1H), 6.43 (d, J=8.4 Hz, 1H), 5.40-5.34 (m, 1H), 2.35 (s, 3H), 2.25 (s, 3H), 1.63 (d, J=6.4 Hz, 3H), 1.54 (s, 9H). MS: m/z 471.1 (M+H⁺).

### Step 2—Synthesis of tert-butyl (R)-2-((1-(3,7-dimethyl-4-oxo-2-(pyridin-3-yl)-4H-pyrido[1,2-a]pyrimidin-9-yl)ethyl)amino)benzoate & tert-butyl (S)-2-((1-(3,7-dimethyl-4-oxo-2-(pyridin-3-yl)-4H-pyrido[1,2-a]pyrimidin-9-yl)ethyl)amino)benzoate

tert-butyl 2-((1-(3,7-dimethyl-4-oxo-2-(pyridin-3-yl)-4H-pyrido[1,2-a]pyrimidin-9-yl)ethyl)amino)benzoate (120 mg, 255 umol) was separated by using chiral SFC (DAICEL CHIRALCEL AD (250 mm*30 mm, 5 um); Supercritical CO₂/EtOH+0.1% NH₃·H₂O=70/30; 60 mL/min) to afford tert-butyl (R)-2-((1-(3,7-dimethyl-4-oxo-2-(pyridin-3-yl)-4H-pyrido[1,2-a]pyrimidin-9-yl)ethyl)amino)benzoate (40 mg, first peak) and tert-butyl (S)-2-((1-(3,7-dimethyl-4-oxo-2-(pyridin-3-yl)-4H-pyrido[1,2-a]pyrimidin-9-yl)ethyl)amino)benzoate (45 mg, second peak) both as white solid. Absolute configuration was arbitrarily assigned to each enantiomer. MS: m/z 471.1 (M+H⁺).

### Step 3—Synthesis of (R)-2-((1-(3,7-dimethyl-4-oxo-2-(pyridin-3-yl)-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl (R)-2-((1-(3,7-dimethyl-4-oxo-2-(pyridin-3-yl)-4H-pyrido[1,2-a]pyrimidin-9-yl)ethyl)amino)benzoate, (R)-2-((1-(3,7-dimethyl-4-oxo-2-(pyridin-3-yl)-4H-pyrido[1,2-a]pyrimidin-9-yl)ethyl)amino)benzoic acid (31 mg, 76%) was obtained as a white solid. ¹H NMR (400 MHz, DMSO-d₆) δ 12.78 (s, 1H), 8.95 (d, J=1.6 Hz, 1H), 8.73-8.67 (m, 2H), 8.49-8.39 (m, 1H), 8.17-8.15 (m, 1H), 7.82-7.80 (m, 1H), 7.68 (d, J=1.6 Hz, 1H), 7.62-7.53 (m, 1H), 7.23-7.12 (m, 1H), 6.56-6.52 (m, 1H), 6.36 (d, J=8.4 Hz, 1H), 5.42-5.36 (m, 1H), 2.34 (s, 3H), 2.25 (s, 3H), 1.60 (d, J=6.8 Hz, 3H). MS: m/z 415.1 (M+H⁺).

### Example 44: Preparation of (R)-2-((1-(2-(6-cyclopropylpyridin-3-yl)-3,7-dimethyl-4-oxo-4H-pyrido[1,2-a]pyrimidin-9-yl)ethyl)amino)benzoic acid (Compound 973)

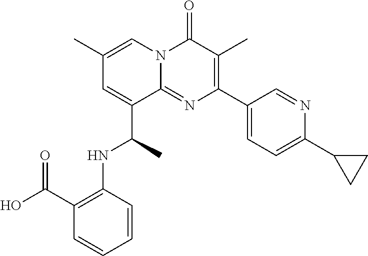 [SMILES: Cc1cc([C@@H](C)Nc2ccccc2C(=O)O)c2nc(-c3ccc(C4CC4)nc3)c(C)c(=O)n2c1]

### Step 1—Synthesis of tert-butyl 2-((1-(2-(6-cyclopropylpyridin-3-yl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (6-cyclopropylpyridin-3-yl)boronic acid, tert-butyl 2-((1-(2-(6-cyclopropylpyridin-3-yl)-3,7-dimethyl-4-oxo-4H-pyrido[1,2-a]pyrimidin-9-yl)ethyl)amino)benzoate (160 mg, 88%) was obtained as a white solid. MS: m/z 511.2 (M+H⁺).

### Step 2—Synthesis of tert-butyl (R)-2-((1-(2-(6-cyclopropylpyridin-3-yl)-3,7-dimethyl-4-oxo-4H-pyrido[1,2-a]pyrimidin-9-yl)ethyl)amino)benzoate & tert-butyl (S)-2-((1-(2-(6-cyclopropylpyridin-3-yl)-3,7-dimethyl-4-oxo-4H-pyrido[1,2-a]pyrimidin-9-yl)ethyl)amino)benzoate

tert-butyl 2-((1-(2-(6-cyclopropylpyridin-3-yl)-3,7-dimethyl-4-oxo-4H-pyrido[1,2-a]pyrimidin-9-yl)ethyl)amino)benzoate (130 mg, 254 umol) was separated by using chiral SFC (DAICEL CHIRALCEL IG (250 mm*30 mm, 5 um); Supercritical CO₂/MeOH+0.1% NH₃·H2O=55/45; 80 mL/min) to afford tert-butyl (R)-2-((1-(2-(6-cyclopropylpyridin-3-yl)-3,7-dimethyl-4-oxo-4H-pyrido[1,2-a]pyrimidin-9-yl)ethyl)amino)benzoate (50 mg, first peak) and tert-butyl (S)-2-((1-(2-(6-cyclopropylpyridin-3-yl)-3,7-dimethyl-4-oxo-4H-pyrido[1,2-a]pyrimidin-9-yl)ethyl)amino)benzoate (50 mg, second peak) both as white solid. Absolute configuration was arbitrarily assigned to each enantiomer. MS: m/z 511.1 (M+H⁺).

### Step 3—Synthesis of (R)-2-((1-(2-(6-cyclopropylpyridin-3-yl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl (R)-2-((1-(2-(6-cyclopropylpyridin-3-yl)-3,7-dimethyl-4-oxo-4H-pyrido[1,2-a]pyrimidin-9-yl)ethyl)amino)benzoate, (R)-2-((1-(2-(6-cyclopropylpyridin-3-yl)-3,7-dimethyl-4-oxo-4H-pyrido[1,2-a]pyrimidin-9-yl)ethyl)amino)benzoic acid (Compound 1079) (16 mg, 36%) was obtained as a white solid. ¹H NMR (400 MHz, DMSO-d₆) δ 12.77 (s, 1H), 8.79 (d, J=2.0 Hz, 1H), 8.70 (s, 1H), 8.42 (d, J=5.6 Hz, 1H), 8.13 (s, 1H), 8.03-8.00 (m, 1H), 7.81-7.79 (m, 1H), 7.66 (d, J=1.6 Hz, 1H), 7.46 (d, J=8.4 Hz, 1H), 7.24-7.15 (m, 1H), 6.56-6.52 (m, 1H), 6.35 (d, J=8.4 Hz, 1H), 5.43-5.37 (m, 1H), 2.33 (s, 3H), 2.25 (s, 3H), 2.23-2.14 (m, 1H), 1.60 (d, J=6.4 Hz, 3H), 1.06-0.93 (m, 4H). MS: m/z 455.1 (M+H⁺).

### Example 45: Preparation of (R)-2-((1-(2-(2-cyclopropylpyridin-4-yl)-3,7-dimethyl-4-oxo-4H-pyrido[1,2-a]pyrimidin-9-yl)ethyl)amino)benzoic acid (Compound 953)

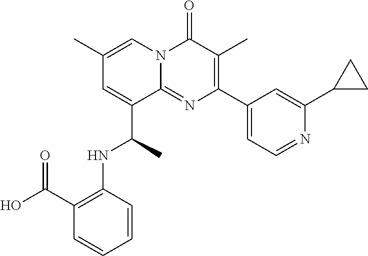 [SMILES: Cc1cc([C@@H](C)Nc2ccccc2C(=O)O)c2nc(-c3ccnc(C4CC4)c3)c(C)c(=O)n2c1]

### Step 1—Synthesis of tert-butyl 2-((1-(2-(2-cyclopropylpyridin-4-yl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with (2-cyclopropyl-4-pyridyl)boronic acid, tert-butyl 2-((1-(2-(2-cyclopropylpyridin-4-yl)-3,7-dimethyl-4-oxo-4H-pyrido[1,2-a]pyrimidin-9-yl)ethyl)amino)benzoate (140 mg, 81%) was obtained as a yellow solid. ¹H NMR (400 MHz, CDCl₃) δ 8.77 (s, 1H), 8.59 (d, J=5.2 Hz, 1H), 8.28 (d, J=5.6 Hz, 1H), 7.89 (dd, J=8.0, 1.6 Hz, 1H), 7.62 (d, J=1.6 Hz, 1H), 7.44 (s, 1H), 7.41-7.35 (m, 1H), 7.17-7.10 (m, 1H), 6.58-6.54 (m, 1H), 6.27 (d, J=8.4 Hz, 1H), 5.51-5.41 (m, 1H), 2.36 (s, 6H), 2.25-2.15 (m, 1H), 1.68 (d, J=6.8 Hz, 3H), 1.63 (s, 9H), 1.16-1.10 (m, 4H). MS: m/z 511.1 (M+H⁺).

### Step 2—Synthesis of tert-butyl (R)-2-((1-(2-(2-cyclopropylpyridin-4-yl)-3,7-dimethyl-4-oxo-4H-pyrido[1,2-a]pyrimidin-9-yl)ethyl)amino)benzoate and tert-butyl (S)-2-((1-(2-(2-cyclopropylpyridin-4-yl)-3,7-dimethyl-4-oxo-4H-pyrido[1,2-a]pyrimidin-9-yl)ethyl)amino)benzoate

tert-butyl 2-((1-(2-(2-cyclopropylpyridin-4-yl)-3,7-dimethyl-4-oxo-4H-pyrido[1,2-a]pyrimidin-9-yl)ethyl)amino)benzoate (120 mg, 235 mol) was separated by using chiral SFC (DAICEL CHIRALPAK IG (250 mm*30 mm, 10 m); Supercritical CO₂/EtOH+0.1% NH₃·H₂O=65/35; 80 mL/min) to afford tert-butyl (R)-2-((1-(2-(2-cyclopropylpyridin-4-yl)-3,7-dimethyl-4-oxo-4H-pyrido[1,2-a]pyrimidin-9-yl)ethyl)amino)benzoate (50 mg, first peak) and tert-butyl (S)-2-((1-(2-(2-cyclopropylpyridin-4-yl)-3,7-dimethyl-4-oxo-4H-pyrido[1,2-a]pyrimidin-9-yl)ethyl)amino)benzoate (50 mg, second peak) both as a white solid. Absolute configuration was arbitrarily assigned to each enantiomer. MS: m/z 511.2 (M+H⁻).

### Step 3—Synthesis of (R)-2-((1-(2-(2-cyclopropylpyridin-4-yl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl (R)-2-((1-(2-(2-cyclopropylpyridin-4-yl)-3,7-dimethyl-4-oxo-4H-pyrido[1,2-a]pyrimidin-9-yl)ethyl)amino)benzoate, (R)-2-((1-(2-(2-cyclopropylpyridin-4-yl)-3,7-dimethyl-4-oxo-4H-pyrido[1,2-a]pyrimidin-9-yl)ethyl)amino)benzoic acid (Compound 1055) (33 mg, 66%) was obtained as a yellow solid. ¹H NMR (400 MHz, DMSO-d₆) δ 12.77 (s, 1H), 8.72 (s, 1H), 8.55 (d, J=5.2 Hz, 1H), 8.45 (s, 1H), 7.85-7.78 (m, 1H), 7.69 (d, J=1.6 Hz, 1H), 7.59 (s, 1H), 7.45-7.40 (m, 1H), 7.21-7.17 (m, 1H), 6.57-6.51 (m, 1H), 6.36 (d, J=8.6 Hz, 1H), 5.55-5.52 (m, 1H), 2.34 (s, 3H), 2.22-2.21 (m, 4H), 1.60 (d, J=6.8 Hz, 3H), 1.01-0.96 (m, 4H). MS: m/z 455.1 (M+H⁺).

### Example 46: Preparation of (S)-2-((1-(2-(5-chloro-6-cyanopyridin-3-yl)-3,7-dimethyl-4-oxo-4H-pyrido[1,2-a]pyrimidin-9-yl)ethyl)amino)benzoic acid (Compound 978)

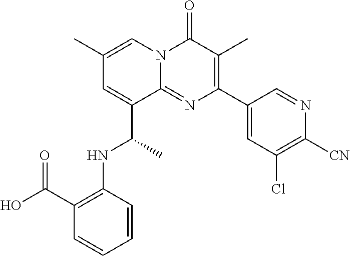 [SMILES: Cc1cc([C@H](C)Nc2ccccc2C(=O)O)c2nc(-c3cnc(C#N)c(Cl)c3)c(C)c(=O)n2c1]

### Step 1—Synthesis of tert-butyl 2-((1-(2-(5-chloro-6-cyanopyridin-3-yl)-3,7-dimethyl-4-oxo-4H-pyrido[1,2-a]pyrimidin-9-yl)ethyl)amino)benzoate

Following the procedure described in Example 33, Step 1 and making non-critical variations as required to replace (3-cyanophenyl)boronic acid with 3-chloro-5-(4,4,5,5-tetramethyl-1,3,2-dioxaborolan-2-yl)picolinonitrile, tert-butyl 2-((1-(2-(5-chloro-6-cyanopyridin-3-yl)-3,7-dimethyl-4-oxo-4H-pyrido[1,2-a]pyrimidin-9-yl)ethyl)amino)benzoate (120 mg, 76%) was obtained as a white solid. MS: m/z 530.2 (M+H⁺).

### Step 2—Synthesis of 2-((1-(2-(5-chloro-6-cyanopyridin-3-yl)-3,7-dimethyl-4-oxo-4H-pyrido[1,2-a]pyrimidin-9-yl)ethyl)amino)benzoic acid

Following the procedure described in Example 33, Step 2 and making non-critical variations as required to replace tert-butyl 2-((1-(2-(3-cyanophenyl)-3,7-dimethyl-4-oxo-4H-pyrido[1,2-a]pyrimidin-9-yl)ethyl)amino)benzoate with tert-butyl 2-((1-(2-(5-chloro-6-cyanopyridin-3-yl)-3,7-dimethyl-4-oxo-4H-pyrido[1,2-a]pyrimidin-9-yl)ethyl)amino)benzoate, 2-((1-(2-(5-chloro-6-cyanopyridin-3-yl)-3,7-dimethyl-4-oxo-4H-pyrido[1,2-a]pyrimidin-9-yl)ethyl)amino)benzoic acid (40 mg, 27%) was obtained as a white solid. MS: m/z 474.1 (M+H⁺).

### Step 3—Synthesis of (R)-2-((1-(2-(5-chloro-6-cyanopyridin-3-yl)-3,7-dimethyl-4-oxo-4H-pyrido[1,2-a]pyrimidin-9-yl)ethyl)amino)benzoic acid & (S)-2-((1-(2-(5-chloro-6-cyanopyridin-3-yl)-3,7-dimethyl-4-oxo-4H-pyrido[1,2-a]pyrimidin-9-yl)ethyl)amino)benzoic acid (Compound 1084)

2-((1-(2-(5-chloro-6-cyanopyridin-3-yl)-3,7-dimethyl-4-oxo-4H-pyrido[1,2-a]pyrimidin-9-yl)ethyl)amino)benzoic acid (40 mg, 84 μmol) was separated by using chiral SFC (DAICEL CHIRALPAK OD-H (250 mm*30 mm, 5 μm); Supercritical CO₂/MeOH+0.1% NH₃·H₂O=50/50; 60 mL/min) to afford (R)-2-((1-(2-(5-chloro-6-cyanopyridin-3-yl)-3,7-dimethyl-4-oxo-4H-pyrido[1,2-a]pyrimidin-9-yl)ethyl)amino)benzoic acid (15 mg, first peak) and (S)-2-((1-(2-(5-chloro-6-cyanopyridin-3-yl)-3,7-dimethyl-4-oxo-4H-pyrido[1,2-a]pyrimidin-9-yl)ethyl)amino)benzoic acid (Compound 1084) (15 mg, second peak) both as white solid. Absolute configuration was arbitrarily assigned to each enantiomer. Second peak: ¹H NMR (400 MHz, DMSO-d₆) δ 9.07 (d, J=2.0 Hz, 1H), 8.74 (s, 1H), 8.61 (d, J=2.0 Hz, 1H), 7.85-7.86 (m, 1H), 7.67 (s, 1H), 7.02-6.99 (m, 1H), 6.47-6.43 (m, 1H), 6.22-6.19 (m, 1H), 5.34-5.31 (m, 1H), 2.33 (s, 3H), 2.25 (s, 3H), 1.56 (d, J=6.8 Hz, 3H). MS: m/z 474.0 (M+H⁺).

### Example 47: Preparation of 9-(1-((3-hydroxybenzo[d]isoxazol-4-yl)amino)ethyl)-3,7-dimethyl-2-(pyridin-3-yl)-4H-pyrido[1,2-a]pyrimidin-4-one (Compound 1365)

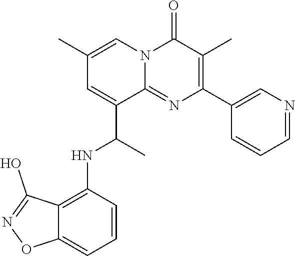 [SMILES: Cc1cc(C(C)Nc2cccc3onc(O)c23)c2nc(-c3cccnc3)c(C)c(=O)n2c1]

### Step 1—Synthesis of 9-chloro-2-hydroxy-3,7-dimethyl-4H-pyrido[1,2-a]pyrimidin-4-one

A solution of 3-chloro-5-methylpyridin-2-amine (5 g, 35 mmol) in acetone (60 mL) was added bis(2,4,6-trichlorophenyl) 2-methylmalonate (18.4 g, 38.6 mmol). The reaction mixture was heated to 60° C. for 2 h under N₂ atmosphere. After cooling to room temperature, the reaction was filtered to give the title compound (6.7 g, 85%) as a yellow solid that required no further purification. ¹H NMR (400 MHz, DMSO-d₆) δ 11.72 (s, 1H), 8.67 (s, 1H), 8.05 (s, 1H), 2.34 (s, 3H), 1.96 (s, 3H).

### Step 2—Synthesis of 9-chloro-3,7-dimethyl-4-oxo-4H-pyrido[1,2-a]pyrimidin-2-yl trifluoromethanesulfonate

To a solution of 9-chloro-2-hydroxy-3,7-dimethyl-4H-pyrido[1,2-a]pyrimidin-4-one (2.7 g, 12 mmol) in DCM (30 mL) was added pyridine (2.9 mL, 36 mmol) and Tf₂₀ (2.38 mL, 14.4 mmol). The reaction was stirred at 0° C. for 1 h. The reaction mixture was diluted with DCM (20 mL) and washed with 1M HCl (30 mL), and then the organic layer was washed with NaHCO₃ (40 mL×2), dried over anhydrous Na₂SO₄ and filtered and concentrated in vacuo to give the title compound (4.2 g, 98%) as a yellow solid that required no further purification. ¹H NMR (400 MHz, DMSO-d₆) δ 8.79 (s, 1H), 8.32 (s, 1H), 2.43 (s, 3H), 2.14 (s, 3H).

### Step 3—Synthesis of 9-chloro-3,7-dimethyl-2-(pyridin-3-yl)-4H-pyrido[1,2-a]pyrimidin-4-one

A mixture of 9-chloro-3,7-dimethyl-4-oxo-4H-pyrido[1,2-a]pyrimidin-2-yl trifluoromethanesulfonate (6 g, 16.8 mmol), pyridin-3-ylboronic acid (2.27 mg, 18.5 mmol), Pd(PPh₃)₄ (972 mg, 841 mol) and Na₂CO₃ (5.35 g, 50.5 mmol) in dioxane (100 mL) and H₂O (10 mL). The mixture was heated to 80° C. for 10 h under N₂ atmosphere. After cooling to room temperature, the reaction mixture was filtered and concentrated in vacuo. The crude residue was purified by silica gel chromatography (solvent gradient: 0-50% EtOAc in petroleum ether) to give the title compound (4.5 g, 86%) as brown solid. ¹H NMR (400 MHz, DMSO-d₆) δ 8.86 (s, 1H), 8.75 (s, 1H), 8.71-8.67 (m, 1H), 8.60 (s, 1H), 8.15-8.11 (m, 1H), 8.09-8.05 (m, 1H), 2.40 (s, 3H), 2.21 (s, 3H). MS: m/z 286.0 (M+H⁺).

### Step 4—Synthesis of 9-acetyl-3,7-dimethyl-2-(pyridin-3-yl)-4H-pyrido[1,2-a]pyrimidin-4-one

A mixture of 9-chloro-3,7-dimethyl-2-(pyridin-3-yl)-4H-pyrido[1,2-a]pyrimidin-4-one (950 mg, 3.32 mmol), tributyl(1-ethoxyvinyl)stannane (3.49 g, 9.66 mmol), SPhos Pd G3 (129.7 mg, 166 umol) and CsF (1.01 g, 6.65 mmol) in toluene (10 mL) was degassed and purged with N₂ for 3 times. The reaction mixture was stirred at 100° C. for 16 h under N₂ atmosphere. After cooling to room temperature, HCl (6 mL, 1 M) was added. The mixture was stirred at room temperature for 0.5 h. The reaction mixture was added 15 mL 10% KF aqueous solution, stirred at room temperature for 2 h. The mixture was extracted with EtOAc (40 mL×3). The combined organic layers were washed with brine (30 mL), dried over anhydrous Na₂SO₄, filtered and concentrated. The crude residue was purified by silica gel chromatography (solvent gradient: 0-50% EtOAc in petroleum ether) to give the title compound (400 mg, 67%) as a yellow solid. ¹H NMR (400 MHz, DMSO-d₆) δ 8.94-8.83 (m, 2H), 8.70-8.67 (m, 1H), 8.11-8.06 (m, 1H), 7.58-7.54 (m, 1H), 2.73 (s, 3H), 2.44 (s, 3H), 2.23 (s, 3H). MS: m/z 294.1 (M+H⁺).

### Step 5—Synthesis of 9-(1-aminoethyl)-3,7-dimethyl-2-(pyridin-3-yl)-4H-pyrido[1,2-a]pyrimidin-4-one

To a solution of 9-acetyl-3,7-dimethyl-2-(pyridin-3-yl)-4H-pyrido[1,2-a]pyrimidin-4-one (200 mg, 681 umol), NH₄OAc (526 mg, 6.8 mmol) and AcOH (41 mg, 681 umol, 236 L) in MeOH (4 mL) was added NaBH₃CN (129 mg, 2.08 mmol). The reaction was heated to 60° C. for 3 h under N₂ atmosphere. After cooling to room temperature, the reaction mixture was diluted with DCM (100 mL). The mixture was washed with saturated NaHCO₃ solution (20 mL×3), brine (30 mL), dried over anhydrous Na₂SO₄ and filtered. The crude residue was purified by silica gel chromatography (solvent gradient: 0-10% DCM in MeOH) to give the title compound (40 mg, 20%) as yellow oil. ¹H NMR (400 MHz, DMSO-d₆) δ 8.91-8.87 (m, 1H), 8.74-8.68 (m, 2H), 8.10 (d, J=8.4 Hz, 1H), 7.91 (s, 1H), 7.61-7.54 (m, 1H), 4.72-4.65 (m, 1H), 2.43 (s, 3H), 2.22 (s, 3H), 1.36 (d, J=6.8 Hz, 3H). MS: m/z 295.1 (M+H⁺).

### Step 6—Synthesis of 4-((1-(3,7-dimethyl-4-oxo-2-(pyridin-3-yl)-4H-pyrido[1,2-a]pyrimidin-9-yl)ethyl)amino)-2-(2,4,6-trimethoxybenzyl)benzo[d]isoxazol-3(2H)-one

A mixture of 9-(1-aminoethyl)-3,7-dimethyl-2-(pyridin-3-yl)-4H-pyrido[1,2-a]pyrimidin-4-one (50 mg, 170 umol), 4-bromo-2-(2,4,6-trimethoxybenzyl)benzo[d]isoxazol-3(2H)-one (56 mg, 142 umol) (prepared according to the procedure in Eur. J. Med Chem. 2018, 150, 930), Pd(OAc)₂ (4 mg, 14 umol), BINAP (11 mg, 17 umol), Cs₂CO₃ (415 mg, 1.3 mmol) in dioxane (2 mL) was stirred at 110° C. for 5 h under N₂ atmosphere. After cooling to room temperature, the reaction mixture was filtered and the filtrate was concentrated in vacuo. The crude residue was purified by silica gel chromatography (solvent gradient: 0-50% EtOAc in petroleum ether) to give the title compound (30 mg, 35%) as yellow oil. MS: m/z 608.1 (M+H⁺).

### Step 7—Synthesis of 9-(1-((3-hydroxybenzo[d]isoxazol-4-yl)amino)ethyl)-3,7-dimethyl-2-(pyridin-3-yl)-4H-pyrido[1,2-a]pyrimidin-4-one

To a solution of 4-((1-(3,7-dimethyl-4-oxo-2-(pyridin-3-yl)-4H-pyrido[1,2-a]pyrimidin-9-yl)ethyl)amino)-2-(2,4,6-trimethoxybenzyl)benzo[d]isoxazol-3(2H)-one (30 mg, 49 umol) in DCM (1.5 mL) was added TFA (0.5 mL) and triisopropylsilane (8.60 mg, 54 mol). The mixture was stirred at room temperature for 16 h. The reaction mixture was concentrated in vacuo. The resulting residue was purified by reverse phase chromatography (acetonitrile 30%-60%/0.225% formic acid in water) to give the title compound (5 mg, 24%) as a white solid. ¹H NMR (400 MHz, DMSO-d₆) δ 8.94 (s, 1H), 8.75-8.67 (m, 2H), 8.17 (d, J=8.0 Hz, 1H), 7.87 (d, J=1.6 Hz, 1H), 7.61-7.55 (m, 1H), 7.16-7.11 (m, 1H), 6.52 (d, J=8.8 Hz, 1H), 6.38-6.33 (m, 1H), 6.01 (d, J=8.4 Hz, 1H), 5.45-5.35 (m, 1H), 2.35 (s, 3H), 2.24 (s, 3H), 1.63 (d, J=6.8 Hz, 3H). MS: m/z 428.0 (M+H⁺).

### Example 48: Preparation of 2-(3-fluorophenyl)-9-(1-((2-(3-hydroxyisoxazol-5-yl)phenyl)amino)ethyl)-3,7-dimethyl-4H-pyrido[1,2-a]pyrimidin-4-one (Compound 1367)

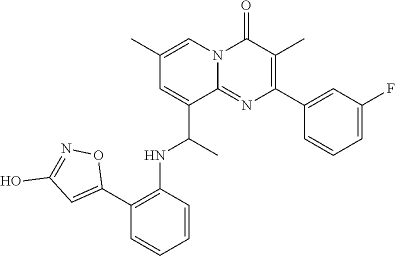 [SMILES: Cc1cc(C(C)Nc2ccccc2-c2cc(O)no2)c2nc(-c3cccc(F)c3)c(C)c(=O)n2c1]

### Step 1—Synthesis of 9-(1-aminoethyl)-2-(3-fluorophenyl)-3,7-dimethyl-4H-pyrido[1,2-a]pyrimidin-4-one

Following the procedure described in Example 47 Step 3—Step 5 and making non-critical variations as required to replace pyridin-3-ylboronic acid with (3-fluorophenyl) boronic acid, the title compound (2 g, crude) was obtained as a yellow solid. MS: m/z 312.1 (M+H⁺).

### Step 2—Synthesis of 9-(1-((2-bromophenyl)amino)ethyl)-2-(3-fluorophenyl)-3,7-dimethyl-4H-pyrido[1,2-a]pyrimidin-4-one

A mixture of 9-(1-aminoethyl)-2-(3-fluorophenyl)-3,7-dimethyl-4H-pyrido[1,2-a]pyrimidin-4-one (1.64 g, 5.3 mmol), 1-bromo-2-iodo-benzene (2.24 g, 7.9 mmol,), Xantphos (610 mg, 1.1 mmol), Pd₂(dba)₃ (482 mg, 527 mol) and Cs₂CO₃ (5.15 g, 15.8 mmol) in dioxane (25 mL) was heated to 100° C. and stirred for 16 h under N₂ atmosphere. After cooling to room temperature, the reaction was quenched with water (30 mL) and extracted with EtOAc (50 mL×3). The combined organic layers were washed with brine (50 mL), dried over anhydrous Na₂SO₄, filtered and concentrated in vacuo. The residue was purified by flash silica gel chromatography (0-30% EtOAc in petroleum ether) to give the title compound (1.1 g, 45%) as a yellow solid. ¹H NMR (400 MHz, DMSO-d₆) δ 8.71 (s, 1H), 7.83 (d, J=1.6 Hz, 1H), 7.59-7.53 (m, 3H), 7.39-7.34 (m, 2H), 7.05-7.01 (m, 1H), 6.55-6.46 (m, 2H), 5.79 (d, J=8.0 Hz, 1H), 5.33-5.26 (m, 1H), 2.36 (s, 3H), 2.22 (s, 3H), 1.63 (d, J=6.4 Hz, 3H). MS: m/z 466.1 (M+H⁺).

### Step 3—Synthesis of methyl 3-(2-((1-(2-(3-fluorophenyl)-3,7-dimethyl-4-oxo-4H-pyrido[1,2-a]pyrimidin-9-yl)ethyl)amino)phenyl)propiolate

A mixture of 9-(1-aminoethyl)-2-(3-fluorophenyl)-3,7-dimethyl-4H-pyrido[1,2-a]pyrimidin-4-one (300 mg, 643 mol), methyl prop-2-ynoate (216 mg, 2.6 mmol), Pd(PPh₃)₂Cl₂ (45 mg, 64 mol), CuI (61 mg, 322 mol) and K₂CO₃ (267 mg, 1.9 mmol) in DMF (5 mL) was heated to 100° C. and stirred for 16 h under N₂ atmosphere. After cooling to room temperature, the reaction was quenched with water (10 mL) and extracted with EtOAc (30 mL×2). The combined organic layers were washed with brine (30 mL), dried over anhydrous Na₂SO₄, filtered and concentrated in vacuo. The residue was purified by flash silica gel chromatography (0-25% EtOAc in petroleum ether) to give the title compound (34 mg, 11%) as yellow oil. ¹H NMR (400 MHz, DMSO-d₆) δ 8.72 (s, 1H), 7.86 (s, 1H), 7.58-7.50 (m, 3H), 7.36 (d, J=7.6 Hz, 2H), 7.19-7.15 (m, 1H), 6.61-6.43 (m, 1H), 6.44 (d, J=8.8 Hz, 1H), 6.02 (d, J=7.6 Hz, 1H), 5.37-5.33 (m, 1H), 3.75 (s, 3H), 2.36 (s, 3H), 2.22 (s, 3H), 1.62 (d, J=6.8 Hz, 3H). MS: m/z 470.3 (M+H⁺).

### Step 4—Synthesis of 2-(3-fluorophenyl)-9-(1-((2-(3-hydroxyisoxazol-5-yl)phenyl)amino)ethyl)-3,7-dimethyl-4H-pyrido[1,2-a]pyrimidin-4-one

To a solution methyl 3-(2-((1-(2-(3-fluorophenyl)-3,7-dimethyl-4-oxo-4H-pyrido[1,2-a]pyrimidin-9-yl)ethyl)amino)phenyl)propiolate (34 mg, 72 μmol) in EtOH (2 mL) was added hydroxylamine (10 mg, 144 mol, 50% in water) slowly. The mixture was stirred at room temperature for 16 h. The reaction mixture was concentrated in vacuo. The resulting residue was purified by reverse phase chromatography (acetonitrile 54%-84%/0.225% formic acid in water) to give the title compound (12 mg, 35%) as a yellow solid. ¹H NMR (400 MHz, DMSO-d₆) δ 8.71 (s, 1H), 7.86 (s, 1H), 7.60-7.56 (m, 1H), 7.54-7.50 (m, 2H), 7.47 (d, J=7.6 Hz, 1H), 7.35-7.31 (m, 1H), 7.11-7.08 (m, 1H), 6.68-6.64 (m, 1H), 6.45-6.41 (m, 2H), 5.99 (d, J=7.2 Hz, 1H), 5.37-5.34 (m, 1H), 2.34 (s, 3H), 2.23 (s, 3H), 1.60 (d, J=6.8 Hz, 3H). MS: m/z 471.1 (M+H⁺).

### Example 49: Preparation of (R)-2-(3-fluorophenyl)-9-(1-((2-(3-hydroxyisoxazol-5-yl)phenyl)amino)ethyl)-3,7-dimethyl-4H-pyrido[1,2-a]pyrimidin-4-one & (S)-2-(3-fluorophenyl)-9-(1-((2-(3-hydroxyisoxazol-5-yl)phenyl)amino)ethyl)-3,7-dimethyl-4H-pyrido[1,2-a]pyrimidin-4-one (Compound 1368 and Compound 1369)

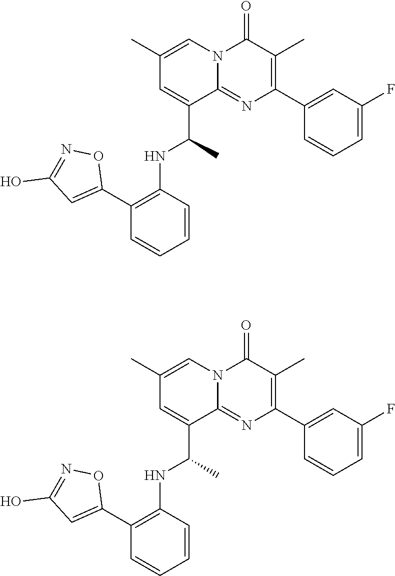 [SMILES: Cc1cc([C@@H](C)Nc2ccccc2-c2cc(O)no2)c2nc(-c3cccc(F)c3)c(C)c(=O)n2c1 ; Cc1cc([C@H](C)Nc2ccccc2-c2cc(O)no2)c2nc(-c3cccc(F)c3)c(C)c(=O)n2c1]

### Step 1—Synthesis of 9-(1-aminoethyl)-2-(3-fluorophenyl)-3,7-dimethyl-4H-pyrido[1,2-a]pyrimidin-4-one

2-(3-fluorophenyl)-9-(1-((2-(3-hydroxyisoxazol-5-yl)phenyl)amino)ethyl)-3,7-dimethyl-4H-pyrido[1,2-a]pyrimidin-4-one (15 mg, 32 μmol) was separated by using chiral SFC (DAICEL CHIRALCEL OD (250 mm*30 mm, 10 um); Supercritical CO₂/MeOH+0.1% NH₃·H₂O=60/40; 80 mL/min) to afford (R)-2-(3-fluorophenyl)-9-(1-((2-(3-hydroxyisoxazol-5-yl)phenyl)amino)ethyl)-3,7-dimethyl-4H-pyrido[1,2-a]pyrimidin-4-one (2.3 mg, first peak) and (S)-2-(3-fluorophenyl)-9-(1-((2-(3-hydroxyisoxazol-5-yl)phenyl)amino)ethyl)-3,7-dimethyl-4H-pyrido[1,2-a]pyrimidin-4-one (2.68 mg, second peak) both as white solid. Absolute configuration was arbitrarily assigned to each enantiomer. First peak: ¹H NMR (400 MHz, DMSO-d₆) δ 8.71 (s, 1H), 7.87 (s, 1H), 7.61-7.56 (m, 1H), 7.55-7.50 (m, 2H), 7.47 (d, J=7.6 Hz, 1H), 7.35-7.31 (m, 1H), 7.11-7.08 (m, 1H), 6.68-6.64 (m, 1H), 6.45-6.41 (m, 1H), 6.45-6.41 (m, 2H), 5.99 (d, J=7.2 Hz, 1H), 5.37-5.34 (m, 1H), 2.34 (s, 3H), 2.23 (s, 3H), 1.61 (d, J=6.8 Hz, 3H). MS: m/z 471.2 (M+H⁺). Second peak: ¹H NMR (400 MHz, DMSO-d₆) δ 8.71 (s, 1H), 7.86 (s, 1H), 7.58-7.56 (m, 1H), 7.54-7.50 (m, 2H), 7.46 (d, J=7.2 Hz, 1H), 7.35-7.31 (m, 1H), 7.11-7.07 (m, 1H), 6.67-6.63 (m, 1H), 6.44-6.39 (m, 2H), 5.98 (d, J=6.8 Hz, 1H), 5.37-5.34 (m, 1H), 2.34 (s, 3H), 2.23 (s, 3H), 1.60 (d, J=6.4 Hz, 3H). MS: m/z 471.2 (M+H⁺).

### Example 50: (R)-2-((1-(3,7-dimethyl-4-oxo-2-((tetrahydro-2H-pyran-4-yl)oxy)-4H-pyrido[1,2-a]pyrimidin-9-yl)ethyl)amino)benzoic acid & (S)-2-((1-(3,7-dimethyl-4-oxo-2-((tetrahydro-2H-pyran-4-yl)oxy)-4H-pyrido[1,2-a]pyrimidin-9-yl)ethyl)amino)benzoic acid (Compounds 1370 and Compound 1371)

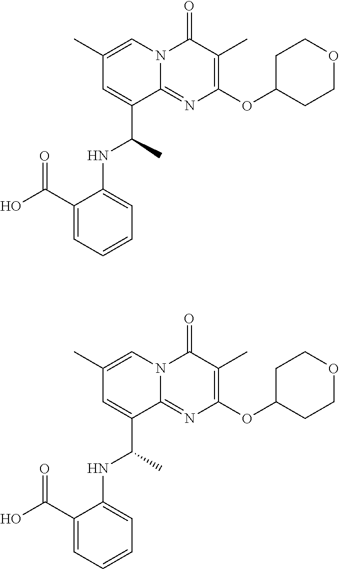 [SMILES: Cc1cc([C@@H](C)Nc2ccccc2C(=O)O)c2nc(OC3CCOCC3)c(C)c(=O)n2c1 ; Cc1cc([C@H](C)Nc2ccccc2C(=O)O)c2nc(OC3CCOCC3)c(C)c(=O)n2c1]

### Step 1—Synthesis of tert-butyl 2-((1-(3,7-dimethyl-4-oxo-2-((tetrahydro-2H-pyran-4-yl)oxy)-4H-pyrido[1,2-a]pyrimidin-9-yl)ethyl)amino)benzoate

To a solution of tert-butyl 2-((1-(2-hydroxy-3,7-dimethyl-4-oxo-4H-pyrido[1,2-a]pyrimidin-9-yl)ethyl)amino)benzoate (500 mg, 1.22 mmol), tetrahydropyran-4-ol (187 mg, 1.83 mmol), PPh₃ (480 mg, 1.83 mmol) in THF (10 mL) was added DEAD (319 mg, 1.83 mmol) at 0° C. under N₂ atmosphere. The mixture was stirred at room temperature for 16 h. The reaction was quenched with water (20 mL). The mixture was extracted with EtOAc (30 mL×2). The combined organic layers were washed with brine (30 mL), dried over anhydrous Na₂SO₄, filtered and concentrated in vacuo. The residue was purified by silica gel chromatography (solvent gradient: 0-20% EtOAc in petroleum ether) to give the title compound (300 mg, 50%) as yellow oil. ¹H NMR (400 MHz, DMSO-d₆) δ 8.69 (s, 1H), 8.18 (d, J=6.4 Hz, 1H), 7.80-7.71 (m, 2H), 7.25-7.16 (m, 1H), 6.58-6.52 (m, 1H), 6.38 (d, J=8.4 Hz, 1H), 5.46-5.36 (m, 1H), 5.28-5.15 (m, 1H), 3.91-3.78 (m, 2H), 3.61-3.47 (m, 2H), 2.32 (s, 3H), 2.06-2.02 (m, 5H), 1.75-1.72 (m, 2H), 1.61 (d, J=6.4 Hz, 3H), 1.56 (s, 9H). MS: m/z 494.3 (M+H⁺).

### Step 2—Synthesis of 2-((1-(3,7-dimethyl-4-oxo-2-((tetrahydro-2H-pyran-4-yl)oxy)-4H-pyrido[1,2-a]pyrimidin-9-yl)ethyl)amino)benzoic acid

To a solution of tert-butyl 2-((1-(3,7-dimethyl-4-oxo-2-((tetrahydro-2H-pyran-4-yl)oxy)-4H-pyrido[1,2-a]pyrimidin-9-yl)ethyl)amino)benzoate (300 mg, 608 mol) in DCM (6 mL) was added TFA (0.5 mL, 6.73 mmol). The reaction mixture was stirred at 40° C. for 16 h. After cooling to room temperature, the reaction mixture was concentrated and the residue was purified by reverse phase chromatography (acetonitrile 52%-82%/0.225% formic acid in water) to give the title compound (225 mg, 84%) as a white solid. MS: m/z 438.2 (M+H⁺).

### Step 3—Synthesis of (R)-2-((1-(3,7-dimethyl-4-oxo-2-((tetrahydro-2H-pyran-4-yl)oxy)-4H-pyrido[1,2-a]pyrimidin-9-yl)ethyl)amino)benzoic acid & (S)-2-((1-(3,7-dimethyl-4-oxo-2-((tetrahydro-2H-pyran-4-yl)oxy)-4H-pyrido[1,2-a]pyrimidin-9-yl)ethyl)amino)benzoic acid

2-((1-(3,7-dimethyl-4-oxo-2-((tetrahydro-2H-pyran-4-yl)oxy)-4H-pyrido[1,2-a]pyrimidin-9-yl)ethyl)amino)benzoic acid (150 mg, 304 mol) was separated using chiral SFC (DAICEL CHIRALPAK AD 250 mm*30 mm, 10 um); Supercritical CO₂/MeOH=60/40; 80 mL/min) to afford (R)-2-((1-(3,7-dimethyl-4-oxo-2-((tetrahydro-2H-pyran-4-yl)oxy)-4H-pyrido[1,2-a]pyrimidin-9-yl)ethyl)amino)benzoic acid (36 mg, first peak) and (S)-2-((1-(3,7-dimethyl-4-oxo-2-((tetrahydro-2H-pyran-4-yl)oxy)-4H-pyrido[1,2-a]pyrimidin-9-yl)ethyl)amino)benzoic acid (34 mg, second peak) as a white solid. Absolute configuration was arbitrarily assigned to each enantiomer. First peak: ¹H NMR (400 MHz, DMSO-d₆) δ 12.74 (s, 1H), 8.69 (s, 1H), 8.41 (d, J=6.0 Hz, 1H), 7.84-7.77 (m, 1H), 7.71 (s, 1H), 7.24-7.16 (m, 1H), 6.58-6.51 (m, 1H), 6.35 (d, J=8.8 Hz, 1H), 5.45-5.35 (m, 1H), 5.27-5.18 (m, 1H), 3.87-3.83 (m, 2H), 3.61-3.48 (m, 2H), 2.31 (s, 3H), 2.05-2.03 (m, 5H), 1.79-1.66 (m, 2H), 1.59 (d, J=6.4 Hz, 3H). MS: m/z 438.1 (M+H⁺). Second peak: ¹H NMR (400 MHz, DMSO-d₆) δ 12.75 (s, 1H), 8.69 (s, 1H), 8.42 (d, J=6.0 Hz, 1H), 7.84-7.78 (m, 1H), 7.71 (s, 1H), 7.25-7.15 (m, 1H), 6.59-6.49 (m, 1H), 6.34 (d, J=8.4 Hz, 1H), 5.46-5.36 (m, 1H), 5.28-5.18 (m, 1H), 3.91-3.80 (m, 2H), 3.60-3.47 (m, 2H), 2.31 (s, 3H), 2.05-2.03 (m, 5H), 1.80-1.67 (m, 2H), 1.59 (d, J=6.8 Hz, 3H). MS: m/z 438.1 (M+H⁺).

### Example 51: (R)-2-(3-fluorophenyl)-9-(1-((3-hydroxybenzo[d]isoxazol-4-yl)amino)ethyl)-3,7-dimethyl-4H-pyrido[1,2-a]pyrimidin-4-one (Compound 1374)

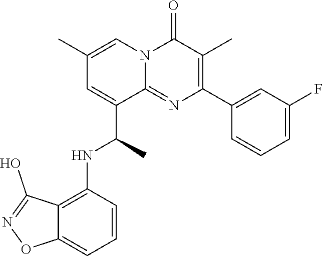 [SMILES: Cc1cc([C@@H](C)Nc2cccc3onc(O)c23)c2nc(-c3cccc(F)c3)c(C)c(=O)n2c1]

### Step 1—Synthesis of 9-chloro-2-(3-fluorophenyl)-3,7-dimethyl-4H-pyrido[1,2-a]pyrimidin-4-one

A mixture of 9-chloro-3,7-dimethyl-4-oxo-4H-pyrido[1,2-a]pyrimidin-2-yl trifluoromethanesulfonate (8 g, 22.4 mmol, prepared according to Example 72, Step 2), (3-fluorophenyl)boronic acid (3.77 mg, 26.9 mmol), Pd(PPh₃)₄ (1.29 g, 1.1 mmol) and K₂CO₃ (6.30 g, 67.3 mmol) in dioxane (80 mL) and H₂O (8 mL). The mixture was heated to 80° C. for 10 h under N₂ atmosphere. After cooling to room temperature, the reaction mixture was quenched with water (20 mL), diluted with EtOAc (140 mL). The mixture was stirred at room temperature for 10 minutes, then the suspension was filtered and the filter cake was dried in vacuo to give the title compound (2.2 g, 32%) was obtained as a yellow solid. ¹H NMR (400 MHz, CDCl₃) δ 8.79 (s, 1H), 7.71 (d, J=1.6 Hz, 1H), 7.49-7.41 (m, 3H), 7.19-7.13 (m, 1H), 2.44 (s, 3H), 2.35 (s, 3H). MS: m/z 303.0 (M+H⁺).

### Step 2—Synthesis of 9-acetyl-2-(3-fluorophenyl)-3,7-dimethyl-pyrido[1,2-a]pyrimidin-4-one

A mixture of 9-chloro-2-(3-fluorophenyl)-3,7-dimethyl-pyrido[1,2-a]pyrimidin-4-one (3.3 g, 10.90 mmol), CsF (3.3 g, 21.80 mmol) and Sphos Pd G3 (850 mg, 1.09 mmol) in toluene (40 mL) was degassed and purged with N₂ for 3 times, and then tributyl(1-ethoxyvinyl)stannane (8.3 g, 22.89 mmol) was added, the final mixture was stirred at 100° C. for 16 h under N₂ atmosphere. After cooling to room temperature, HCl (8 mL, 1 M) was added. The mixture was stirred at room temperature for 0.5 h. The reaction mixture was added 80 mL 10% KF aqueous solution, stirred at room temperature for 2 h. The mixture was extracted with EtOAc (50 mL×3). The combined organic layers were washed with brine (50 mL), dried over anhydrous Na₂SO₄, filtered and concentrated in vacuo. The crude residue was purified by silica gel chromatography (solvent gradient: 0-20% EtOAc in petroleum ether) to give the title compound (2.34 g, 85%) as a yellow solid. ¹H NMR (400 MHz, CDCl₃) δ 8.96 (s, 1H), 7.92 (d, J=2.0 Hz, 1H), 7.48-7.41 (m, 2H), 7.39-7.34 (m, 1H), 7.20-7.14 (m, 1H), 2.88 (s, 3H), 2.47 (s, 3H), 2.36 (s, 3H). MS: m/z 311.0 (M+H⁺).

### Step 3—Synthesis of 9-(1-aminoethyl)-2-(3-fluorophenyl)-3,7-dimethyl-pyrido[1,2-a]pyrimidin-4-one

To a solution of 9-acetyl-2-(3-fluorophenyl)-3,7-dimethyl-pyrido[1,2-a]pyrimidin-4-one (2.34 g, 7.5 mmol), NH₄OAc (8.72 g, 113.1 mmol) and AcOH (452.83 mg, 7.5 mmol, 432 L) in MeOH (40 mL) was added NaBH₃CN (1.42 g, 22.6 mmol). The reaction was heated to 60° C. for 3 h under N₂ atmosphere. After cooling to room temperature, the reaction mixture was diluted with EtOAc (100 mL). The mixture was washed with saturated NaHCO₃ solution (30 mL×3), brine (30 mL), dried over anhydrous Na₂SO₄ and filtered. The crude residue was purified by silica gel chromatography (solvent gradient: 0-15% MeOH in EtOAc (contain 0.1% NH₃·H₂O)) to give the title compound (700 mg, 30%) as yellow oil. ¹H NMR (400 MHz, CDCl₃) δ 8.78 (s, 1H), 7.61 (d, J=1.6 Hz, 1H), 7.45-7.41 (m, 1H), 7.37 (m, 1H), 7.34-7.28 (m, 1H), 7.20-7.06 (m, 1H), 4.83-4.78 (m, 1H), 2.43 (s, 3H), 2.34 (s, 3H), 1.54 (d, J=6.8 Hz, 3H). MS: m/z 312.1 (M+H⁺).

### Step 4—Synthesis of 4-((1-(2-(3-fluorophenyl)-3,7-dimethyl-4-oxo-4H-pyrido[1,2-a]pyrimidin-9-yl)ethyl)amino)-2-(2,4,6-trimethoxybenzyl)benzo[d]isoxazol-3(2H)-one

A mixture of 9-(1-aminoethyl)-2-(3-fluorophenyl)-3,7-dimethyl-pyrido[1,2-a]pyrimidin-4-one (221 mg, 710 μmol), 4-bromo-2-(2,4,6-trimethoxybenzyl)benzo[d]isoxazol-3(2H)-one (200 mg, 507 μmol) (prepared according to the procedure in Eur. J. Med. Chem. 2018, 150, 930), Pd(OAc)₂ (5.7 mg, 25 μmol), BINAP (19.0 mg, 30 μmol), Cs₂CO₃ (1.49 g, 4.57 mmol) in dioxane (2 mL) was stirred at 110° C. for 5 h under N₂ atmosphere. After cooling to room temperature, the reaction mixture was filtered and the filtrate was concentrated in vacuo. The crude residue was purified by silica gel chromatography (solvent gradient: 0-20% EtOAc in petroleum ether) to give the title compound (130 mg, 41%) as yellow oil. MS: m/z 625.1 (M+H⁺).

### Step 5—Synthesis of (R)-4-((1-(2-(3-fluorophenyl)-3,7-dimethyl-4-oxo-4H-pyrido[1,2-a]pyrimidin-9-yl)ethyl)amino)-2-(2,4,6-trimethoxybenzyl)benzo[d]isoxazol-3(2H)-one & (S)-4-((1-(2-(3-fluorophenyl)-3,7-dimethyl-4-oxo-4H-pyrido[1,2-a]pyrimidin-9-yl)ethyl)amino)-2-(2,4,6-trimethoxybenzyl)benzo[d]isoxazol-3(2H)-one

4-((1-(2-(3-fluorophenyl)-3,7-dimethyl-4-oxo-4H-pyrido[1,2-a]pyrimidin-9-yl)ethyl)amino)-2-(2,4,6-trimethoxybenzyl)benzo[d]isoxazol-3(2H)-one (70 mg, 112 mol) was separated by using chiral SFC (DAICEL CHIRALCEL OJ-H (250 mm*30 mm, 10 um); Supercritical CO₂/EtOH+0.1% NH₃·H₂O=50/50; 80 mL/min) to afford (R)-4-((1-(2-(3-fluorophenyl)-3,7-dimethyl-4-oxo-4H-pyrido[1,2-a]pyrimidin-9-yl)ethyl)amino)-2-(2,4,6-trimethoxybenzyl)benzo[d]isoxazol-3(2H)-one (22 mg, first peak) and (S)-4-((1-(2-(3-fluorophenyl)-3,7-dimethyl-4-oxo-4H-pyrido[1,2-a]pyrimidin-9-yl)ethyl)amino)-2-(2,4,6-trimethoxybenzyl)benzo[d]isoxazol-3(2H)-one (23 mg, second peak) both as white solid. Absolute configuration was arbitrarily assigned to each enantiomer. MS: m/z 625.1 (M+H⁺).

### Step 6—Synthesis of (R)-2-(3-fluorophenyl)-9-(1-((3-hydroxybenzo[d]isoxazol-4-yl)amino)ethyl)-3,7-dimethyl-4H-pyrido[1,2-a]pyrimidin-4-one

To a solution of (R)-4-((1-(2-(3-fluorophenyl)-3,7-dimethyl-4-oxo-4H-pyrido[1,2-a]pyrimidin-9-yl)ethyl)amino)-2-(2,4,6-trimethoxybenzyl)benzo[d]isoxazol-3(2H)-one (22 mg, 35 μmol) in DCM (1 mL) was added TFA (0.5 mL) and triisopropylsilane (6.3 mg, 39 μmol). The mixture was stirred at room temperature for 16 h. The reaction mixture was concentrated in vacuo. The resulting residue was purified by reverse phase chromatography (acetonitrile 53%-83%/0.225% formic acid in water) to give the title compound (4.7 mg, 28%) as a white solid. ¹H NMR (400 MHz, DMSO-d₆) δ 12.62 (s, 1H), 8.71 (s, 1H), 7.87 (d, J=1.2 Hz, 1H), 7.61-7.53 (m, 3H), 7.37-7.32 (m, 1H), 7.22-7.12 (m, 1H), 6.55 (m, 1H), 6.03 (d, J=8.0 Hz, 1H), 5.42-5.34 (m, 1H), 2.35 (s, 3H), 2.23 (s, 3H), 1.63 (d, J=6.8 Hz, 3H). MS: m/z 445.1 (M+H⁺).

### Example 52: 2-(3-fluorophenyl)-9-(1-((2-(3-hydroxyoxetan-3-yl)phenyl)amino)ethyl)-3,7-dimethyl-4H-pyrido[1,2-a]pyrimidin-4-one (Compound 1389)

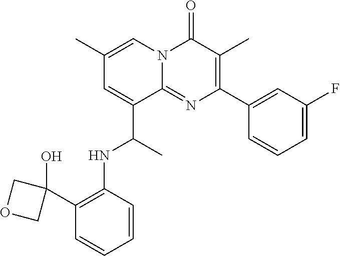 [SMILES: Cc1cc(C(C)Nc2ccccc2C2(O)COC2)c2nc(-c3cccc(F)c3)c(C)c(=O)n2c1]

### Step 1—Synthesis of 2-(3-fluorophenyl)-9-(1-((2-iodophenyl)amino)ethyl)-3,7-dimethyl-4H-pyrido[1,2-a]pyrimidin-4-one

A mixture of 9-(1-aminoethyl)-2-(3-fluorophenyl)-3,7-dimethyl-4H-pyrido[1,2-a]pyrimidin-4-one (3.3 g, 10.6 mmol), 1,2-diiodobenzene (6.99 g, 21.2 mmol), Xantphos (613 mg, 1.1 mmol), Pd₂(dba)₃ (971 mg, 1.1 mmol) and Cs₂CO₃ (10.4 g, 31.8 mmol) in dioxane (80 mL) was heated to 100° C. and stirred for 16 h under N₂ atmosphere. After cooling to room temperature, the reaction was quenched with water (50 mL) and extracted with EtOAc (100 mL×3). The combined organic layers were washed with brine (50 mL), dried over anhydrous Na₂SO₄, filtered and concentrated in vacuo. The residue was purified by flash silica gel chromatography (0-10% EtOAc in petroleum ether) to give the title compound (0.8 g, 15%) as a yellow solid. MS: m/z 514.0 (M+H⁺).

### Step 2—Synthesis of 2-(3-fluorophenyl)-9-(1-((2-(3-hydroxyoxetan-3-yl)phenyl)amino)ethyl)-3,7-dimethyl-4H-pyrido[1,2-a]pyrimidin-4-one

To a solution of 2-(3-fluorophenyl)-9-(1-((2-iodophenyl)amino)ethyl)-3,7-dimethyl-4H-pyrido[1,2-a]pyrimidin-4-one (0.14 g, 0.27 mmol) and oxetan-3-one (37 mg, 0.51 mmol) in THF (4 mL) was added n-BuLi (0.11 ml, 0.27 mmol, 2.5 M in n-hexane) at −78° C. under N₂ atmosphere. The mixture was stirred at room temperature for 3 h under N₂ atmosphere. The mixture was quenched with NH₄Cl (10 mL) at 0° C., diluted with water (20 mL), extracted with EtOAc (30 mL×2). The combined organic layers were washed with brine (30 mL), dried over anhydrous Na₂SO₄, filtered and concentrated in vacuo. The residue was purified by reverse phase chromatography (acetonitrile 68-98%/0.225% formic acid in water) to give the title compound (2.6 mg, 2%) as a yellow solid. ¹H NMR (400 MHz, DMSO-d₆) δ 8.71 (s, 1H), 7.65 (s, 1H), 7.61-7.55 (m, 3H), 7.39-7.31 (m, 1H), 7.23 (d, J=7.6 Hz, 1H), 7.00-6.92 (m, 1H), 6.66-6.57 (m, 2H), 6.27 (d, J=8.0 Hz, 1H), 5.50 (d, J=6.8 Hz, 1H), 5.33-5.25 (m, 1H), 5.04-4.94 (m, 2H), 4.86-4.77 (m, 2H), 2.34 (s, 3H), 2.24 (s, 3H), 1.53 (d, J=6.8 Hz, 3H). MS: m/z 460.1 (M+H⁺).

### Example 53: (S)-2-(3-fluorophenyl)-9-(1-(2-(3-hydroxy-1,1-dioxidothietan-3-yl)phenoxy)ethyl)-3,7-dimethyl-4H-pyrido[1,2-a]pyrimidin-4-one (Compound 1393)

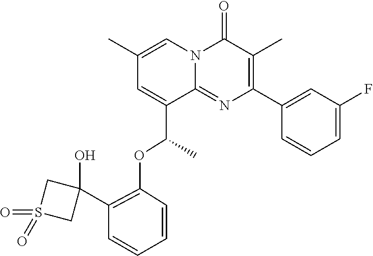 [SMILES: Cc1cc([C@H](C)Oc2ccccc2C2(O)CS(=O)(=O)C2)c2nc(-c3cccc(F)c3)c(C)c(=O)n2c1]

### Step 1—Synthesis of 2-(3-fluorophenyl)-9-(1-hydroxyethyl)-3,7-dimethyl-4H-pyrido[1,2-a]pyrimidin-4-one

To a solution of 9-acetyl-2-(3-fluorophenyl)-3,7-dimethyl-4H-pyrido[1,2-a]pyrimidin-4-one (1 g, 3.22 mmol) in MeOH (20 mL) was added NaBH₄ (180 mg, 4.76 mmol) at 0° C. The mixture was stirred at room temperature for 3 h under N₂ atmosphere. The mixture was quenched with NH₄Cl (10 mL) at 0° C., diluted with water (20 mL), extracted with EtOAc (30 mL×2). The combined organic layers were washed with brine (30 mL), dried over anhydrous Na₂SO₄, filtered and concentrated in vacuo. The crude residue was purified by silica gel chromatography (solvent gradient: 0-50% EtOAc in petroleum ether) to give the title compound (0.68 g, 68%) as a yellow solid. ¹H NMR (400 MHz, CDCl₃) δ 8.79 (s, 1H), 7.50-7.43 (m, 2H), 7.40-7.36 (m, 1H), 7.34-7.28 (m, 1H), 7.20-7.14 (m, 1H), 5.33-5.18 (m, 2H), 2.44 (s, 3H), 2.32 (s, 3H), 1.68 (d, J=6.4 Hz, 3H). MS: m/z 313.0 (M+H⁺).

### Step 2—Synthesis of 2-(3-fluorophenyl)-9-(1-(2-iodophenoxy)ethyl)-3,7-dimethyl-4H-pyrido[1,2-a]pyrimidin-4-one

To a solution of 2-(3-fluorophenyl)-9-(1-hydroxyethyl)-3,7-dimethyl-4H-pyrido[1,2-a]pyrimidin-4-one (680 mg, 2.18 mmol) and 2-iodophenol (718 mg, 3.27 mmol) in THF (10 mL) was added PPh₃ (857 mg, 3.27 mmol) and DIAD (660 mg, 3.27 mmol) at 0° C. The mixture was stirred at room temperature for 16 h under N₂ atmosphere. The solution was concentrated in vacuo. The crude residue was purified by silica gel chromatography (solvent gradient: 0-30% EtOAc in petroleum ether) to give the title compound (0.6 g, 54%) as a yellow solid. ¹H NMR (400 MHz, DMSO-d₆) δ 8.75 (s, 1H), 7.83-7.76 (m, 2H), 7.62-7.47 (m, 3H), 7.36-7.32 (m, 1H), 7.25-7.19 (m, 1H), 6.84 (d, J=7.8 Hz, 1H), 6.73-6.69 (m, 1H), 6.23-6.17 (m, 1H), 2.40 (s, 3H), 2.22 (s, 3H), 1.70 (d, J=6.4 Hz, 3H). MS: m/z 515.0 (M+H⁺).

### Step 3—Synthesis of 2-(3-fluorophenyl)-9-(1-(2-(3-hydroxythietan-3-yl)phenoxy)ethyl)-3,7-dimethyl-4H-pyrido[1,2-a]pyrimidin-4-one

To a solution of 2-(3-fluorophenyl)-9-(1-(2-iodophenoxy)ethyl)-3,7-dimethyl-4H-pyrido[1,2-a]pyrimidin-4-one (0.6 g, 1.17 mmol) and thietan-3-one (204 mg, 2.31 mmol) in THE (20 mL) was added n-BuLi (0.47 ml, 1.17 mmol, 2.5 M in n-hexane) at −78° C. under N₂ atmosphere. The mixture was stirred at room temperature for 3 h under N₂ atmosphere. The mixture was quenched with NH₄Cl (10 mL) at 0° C., diluted with water (20 mL), extracted with EtOAc (30 mL×2). The combined organic layers were washed with brine (30 mL), dried over anhydrous Na₂SO₄, filtered and concentrated in vacuo. The crude residue was purified by silica gel chromatography (solvent gradient: 0-30% EtOAc in petroleum ether) to give the title compound (120 mg, 22%) as a yellow solid. ¹H NMR (400 MHz, DMSO-d₆) δ 8.71 (s, 1H), 7.66 (d, J=1.6 Hz, 1H), 7.62-7.53 (m, 3H), 7.42-7.31 (m, 2H), 6.99-6.93 (m, 1H), 6.79 (s, 1H), 6.63-6.58 (m, 1H), 6.25 (d, J=8.0 Hz, 1H), 6.09 (d, J=5.6 Hz, 1H), 5.54-5.25 (m, 1H), 3.74-3.61 (m, 4H), 2.33 (s, 3H), 2.24 (s, 3H), 1.53 (d, J=6.4 Hz, 3H). MS: m/z 476.1 (M+H⁺).

### Step 4—Synthesis of 2-(3-fluorophenyl)-9-(1-(2-(3-hydroxy-1,1-dioxidothietan-3-yl)phenoxy)ethyl)-3,7-dimethyl-4H-pyrido[1,2-a]pyrimidin-4-one

To a solution of 2-(3-fluorophenyl)-9-(1-(2-(3-hydroxythietan-3-yl)phenoxy)ethyl)-3,7-dimethyl-4H-pyrido[1,2-a]pyrimidin-4-one (100 mg, 209 umol) in EtOH (4 mL) and H₂O (1 mL) was added oxone (516 mg, 838 umol) at 0° C. The mixture was stirred at room temperature for 3 h. The mixture was quenched with NaHCO₃ (10 mL) at 0° C., diluted with water (20 mL), extracted with EtOAc (30 mL×2). The combined organic layers were washed with brine (30 mL), dried over anhydrous Na₂SO₄, filtered and concentrated in vacuo. The residue was purified by reverse phase chromatography (acetonitrile 60-90%/0.225% formic acid in water) to give the title compound (30 mg, 28%) as a white solid. ¹H NMR (400 MHz, DMSO-d₆) δ 8.75 (s, 1H), 7.86 (d, J=1.6 Hz, 1H), 7.63-7.43 (m, 4H), 7.38-7.29 (m, 1H), 7.17-7.11 (m, 1H), 6.95-6.89 (m, 1H), 6.71 (d, J=8.4 Hz, 1H), 6.53 (s, 1H), 6.24-6.16 (m, 1H), 5.09-4.94 (m, 2H), 4.26-4.12 (m, 2H), 2.38 (s, 3H), 2.24 (s, 3H), 1.73 (d, J=6.4 Hz, 3H). MS: m/z 531.1 (M+Na⁺).

### Step 5—Synthesis of (R)-2-(3-fluorophenyl)-9-(1-(2-(3-hydroxy-1,1-dioxidothietan-3-yl)phenoxy)ethyl)-3,7-dimethyl-4H-pyrido[1,2-a]pyrimidin-4-one & (S)-2-(3-fluorophenyl)-9-(1-(2-(3-hydroxy-1,1-dioxidothietan-3-yl)phenoxy)ethyl)-3,7-dimethyl-4H-pyrido[1,2-a]pyrimidin-4-one

2-(3-fluorophenyl)-9-(1-(2-(3-hydroxy-1,1-dioxidothietan-3-yl)phenoxy)ethyl)-3,7-dimethyl-4H-pyrido[1,2-a]pyrimidin-4-one (30 mg, 59 umol) was separated by using chiral SFC (DAICEL CHIRALCEL AS (250 mm*30 mm, 5 um); Supercritical CO₂/EtOH+0.1% NH₃·H₂O=25/75; 60 mL/min) to afford (R)-2-(3-fluorophenyl)-9-(1-(2-(3-hydroxy-1,1-dioxidothietan-3-yl)phenoxy)ethyl)-3,7-dimethyl-4H-pyrido[1,2-a]pyrimidin-4-one (5.4 mg, first peak) and (S)-2-(3-fluorophenyl)-9-(1-(2-(3-hydroxy-1,1-dioxidothietan-3-yl)phenoxy)ethyl)-3,7-dimethyl-4H-pyrido[1,2-a]pyrimidin-4-one (15 mg, second peak) both as white solid. Absolute configuration was arbitrarily assigned to each enantiomer. First peak: ¹H NMR (400 MHz, DMSO-d₆) δ 8.75 (s, 1H), 7.86 (d, J=1.6 Hz, 1H), 7.61-7.45 (m, 4H), 7.38-7.28 (m, 1H), 7.19-7.11 (m, 1H), 6.95-6.88 (m, 1H), 6.70 (d, J=8.4 Hz, 1H), 6.55 (s, 1H), 6.24-6.16 (m, 1H), 5.11-4.91 (m, 2H), 4.23-4.10 (m, 2H), 2.37 (s, 3H), 2.24 (s, 3H), 1.73 (d, J=6.4 Hz, 3H). MS: m/z 531.1 (M+Na⁺). Second peak: 1H NMR (400 MHz, DMSO-d₆) δ 8.75 (s, 1H), 7.86 (d, J=1.6 Hz, 1H), 7.63-7.44 (m, 4H), 7.39-7.30 (m, 1H), 7.18-7.11 (m, 1H), 6.95-6.89 (m, 1H), 6.70 (d, J=8.0 Hz, 1H), 6.54 (s, 1H), 6.24-6.17 (m, 1H), 5.12-4.90 (m, 2H), 4.25-4.12 (m, 2H), 2.38 (s, 3H), 2.24 (s, 3H), 1.73 (d, J=6.4 Hz, 3H). MS: m/z 531.1 (M+Na⁺).

Compounds of Formula (I) in Table A can be prepared analogously to Examples 1-53.

Compounds of Formula (I) in Table B below were prepared analogously to Examples 1-53.

TABLE B

| Cmpd No. | Characterization Data |
|---|---|
| 7 | LC-MS: (M + H)+ found 407.3 |
| 140 | LC-MS: (M + H)+ found 425.1 |
| 150 | LC-MS: (M + H)+ found 423.1 |
| 231 | LC-MS: (M + H)+ found 584.1 |
| 240 | LC-MS: (M + H)+ found 425.3 |
| 267 | LC-MS: (M + H)+ found 401.1 |
| 269 | LC-MS: (M + H)+ found 401.1 |
| 317 | LC-MS: (M + H)+ found 408.1 |
| 347 | LC-MS: (M + H)+ found 469.1 |
| 359 | LC-MS: (M + H)+ found 421.2 |
| 361 | LC-MS: (M + H)+ found 435.1 |
| 363 | LC-MS: (M + H)+ found 413.2 |
| 364 | LC-MS: (M + H)+ found 413.4 |
| 374 | LC-MS: (M + H)+ found 423.1 |
| 381 | LC-MS: (M + H)+ found 471.1 |
| 393 | LC-MS: (M + H)+ found 441.2 |
| 420 | LC-MS: (M + H)+ found 584.1 |
| 422 | LC-MS: (M + H)+ found 401.1 |
| 424 | LC-MS: (M + H)+ found 418.1 |
| 431 | LC-MS: (M + H)+ found 451.1 |
| 435 | LC-MS: (M + H)+ found 431.2 |
| 457 | LC-MS: (M + H)+ found 497.1 |
| 478 | LC-MS: (M + H)+ found 455.1 |
| 512 | LC-MS: (M + H)+ found 435.1 |
| 536 | LC-MS: (M + H)+ found 469.1 |
| 545 | LC-MS: (M + H)+ found 473.2 |
| 570 | LC-MS: (M + H)+ found 506.1 |
| 583 | LC-MS: (M + H)+ found 469.2 |
| 593 | LC-MS: (M + H)+ found 473.2 |
| 595 | LC-MS: (M + H)+ found 470.1 |
| 605 | LC-MS: (M + H)+ found 475.2 |
| 611 | LC-MS: (M + H)+ found 473.2 |
| 620 | LC-MS: (M + H)+ found 440.1 |
| 622 | LC-MS: (M + H)+ found 434.1 |
| 623 | LC-MS: (M + H)+ found 434.1 |
| 625 | LC-MS: (M + H)+ found 464.1 |
| 626 | LC-MS: (M + H)+ found 464.1 |
| 629 | LC-MS: (M + H)+ found 400.2 |
| 632 | LC-MS: (M + H)+ found 482.1 |
| 652 | LC-MS: (M + H)+ found 454.1 |
| 653 | LC-MS: (M + H)+ found 455.1 |
| 658 | LC-MS: (M + H)+ found 471.2 |
| 660 | LC-MS: (M + H)+ found 442.2 |
| 662 | LC-MS: (M + H)+ found 446.1 |
| 696 | LC-MS: (M + H)+ found 469.1 |
| 711 | LC-MS: (M + H)+ found 471.1 |
| 719 | LC-MS: (M + H)+ found 475.1 |
| 722 | LC-MS: (M + H)+ found 483.1 |
| 724 | LC-MS: (M + H)+ found 522.1 |
| 725 | LC-MS: (M + H)+ found 468.1 |
| 729 | LC-MS: (M + H)+ found 472.1 |
| 730 | LC-MS: (M + H)+ found 472.3 |
| 736 | LC-MS: (M + H)+ found 494.1 |
| 737 | LC-MS: (M + H)+ found 522.2 |
| 740 | LC-MS: (M + H)+ found 468.1 |
| 748 | LC-MS: (M + H)+ found 494.1 |
| 758 | LC-MS: (M + H)+ found 483.1 |
| 762 | LC-MS: (M + H)+ found 483.1 |
| 765 | LC-MS: (M + H)+ found 469.1 |
| 767 | LC-MS: (M + H)+ found 483.1 |
| 769 | LC-MS: (M + H)+ found 504.1 |
| 775 | LC-MS: (M + H)+ found 481.2 |
| 781 | LC-MS: (M + H)+ found 471.4 |
| 785 | LC-MS: (M + H)+ found 483.1 |
| 798 | LC-MS: (M + H)+ found 467.1 |
| 804 | LC-MS: (M + H)+ found 496.1 |
| 807 | LC-MS: (M + H)+ found 504.3 |
| 809 | LC-MS: (M + H)+ found 472.4 |
| 818 | LC-MS: (M + H)+ found 468.1 |
| 819 | LC-MS: (M + H)+ found 468.1 |
| 821 | LC-MS: (M + H)+ found 476.1 |
| 822 | LC-MS: (M + H)+ found 476.2 |
| 830 | LC-MS: (M + H)+ found 471.2 |
| 833 | LC-MS: (M + H)+ found 490.1 |
| 848 | LC-MS: (M + H)+ found 489.1 |
| 851 | LC-MS: (M + H)+ found 442.1 |
| 853 | LC-MS: (M + H)+ found 490.1 |
| 855 | LC-MS: (M + H)+ found 465.1 |
| 856 | LC-MS: (M + H)+ found 490.2 |
| 870 | LC-MS: (M + H)+ found 495.1 |
| 875 | LC-MS: (M + H)+ found 515.2 |
| 877 | LC-MS: (M + H)+ found 465.1 |
| 881 | LC-MS: (M + H)+ found 483.1 |
| 890 | LC-MS: (M + H)+ found 494.2 |
| 892 | LC-MS: (M + H)+ found 508.1 |
| 896 | LC-MS: (M + H)+ found 474.1 |
| 899 | LC-MS: (M + H)+ found 457.2 |
| 902 | LC-MS: (M + H)+ found 481.2 |
| 908 | LC-MS: (M + H)+ found 491.1 |
| 915 | LC-MS: (M + H)+ found 451.1 |
| 916 | LC-MS: (M + H)+ found 463.1 |
| 919 | LC-MS: (M + H)+ found 511.2 |
| 922 | LC-MS: (M + H)+ found 495.3 |
| 927 | LC-MS: (M + H)+ found 482.1 |
| 931 | LC-MS: (M + H)+ found 508.1 |
| 934 | LC-MS: (M + H)+ found 624.1 |
| 937 | LC-MS: (M + H)+ found 488.4 |
| 938 | LC-MS: (M + H)+ found 455.1 |
| 943 | LC-MS: (M + H)+ found 476.1 |
| 945 | LC-MS: (M + H)+ found 487.1 |
| 947 | LC-MS: (M + H)+ found 457.1 |
| 949 | LC-MS: (M + H)+ found 455.1 |
| 951 | LC-MS: (M + H)+ found 463.2 |
| 959 | LC-MS: (M + H)+ found 451.2 |
| 961 | LC-MS: (M + H)+ found 543.3 |
| 963 | LC-MS: (M + H)+ found 448.2 |
| 965 | LC-MS: (M + H)+ found 466.1 |
| 967 | LC-MS: (M + H)+ found 439.1 |
| 971 | LC-MS: (M + H)+ found 454.1 |
| 973 | LC-MS: (M + H)+ found 455.1 |
| 975 | LC-MS: (M + H)+ found 440.2 |
| 978 | LC-MS: (M + H)+ found 474.1 |
| 982 | LC-MS: (M + H)+ found 481.1 |
| 984 | LC-MS: (M + H)+ found 481.1 |
| 987 | LC-MS: (M + H)+ found 518.1 |
| 994 | LC-MS: (M + H)+ found 507.1 |
| 999 | LC-MS: (M + H)+ found 489.1 |
| 1008 | LC-MS: (M + H)+ found 550.1 |
| 1015 | LC-MS: (M + H)+ found 495.1 |
| 1019 | LC-MS: (M + H)+ found 495.1 |
| 1021 | LC-MS: (M + H)+ found 488.1 |
| 1022 | LC-MS: (M + H)+ found 446.2 |
| 1026 | LC-MS: (M + H)+ found 519.2 |
| 1027 | LC-MS: (M + H)+ found 543.1 |
| 1031 | LC-MS: (M + H)+ found 492.1 |
| 1033 | LC-MS: (M + H)+ found 474.3 |
| 1034 | LC-MS: (M + H)+ found 474.1 |
| 1036 | LC-MS: (M + H)+ found 489.1 |
| 1038 | LC-MS: (M + H)+ found 443.1 |
| 1042 | LC-MS: (M + H)+ found 473.2 |
| 1048 | LC-MS: (M + H)+ found 519.1 |
| 1050 | LC-MS: (M + H)+ found 482.2 |
| 1052 | LC-MS: (M + H)+ found 481.1 |
| 1055 | LC-MS: (M + H)+ found 422.1 |
| 1058 | LC-MS: (M + H)+ found 457.1 |
| 1059 | LC-MS: (M + H)+ found 517.3 |
| 1063 | LC-MS: (M + H)+ found 454.1 |
| 1064 | LC-MS: (M + H)+ found 490.1 |
| 1066 | LC-MS: (M + H)+ found 499.2 |
| 1067 | LC-MS: (M + H)+ found 429.2 |
| 1069 | LC-MS: (M + H)+ found 445.1 |
| 1070 | LC-MS: (M + H)+ found 444.2 |
| 1074 | LC-MS: (M + H)+ found 598.2 |
| 1081 | LC-MS: (M + H)+ found 493.1 |
| 1083 | LC-MS: (M + H)+ found 508.3 |
| 1090 | LC-MS: (M + H)+ found 454.1 |
| 1092 | LC-MS: (M + H)+ found 456.1 |
| 1096 | LC-MS: (M + H)+ found 490.1 |
| 1097 | LC-MS: (M + H)+ found 499.3 |
| 1099 | LC-MS: (M + H)+ found 463.1 |
| 1101 | LC-MS: (M + H)+ found 445.1 |
| 1103 | LC-MS: (M + H)+ found 457.1 |
| 1104 | LC-MS: (M + H)+ found 457.3 |
| 1105 | LC-MS: (M + H)+ found 497.1 |
| 1107 | LC-MS: (M + H)+ found 450.1 |
| 1109 | LC-MS: (M + H)+ found 490.1 |
| 1111 | LC-MS: (M + H)+ found 441.1 |
| 1114 | LC-MS: (M + H)+ found 450.1 |
| 1115 | LC-MS: (M + H)+ found 446.2 |
| 1118 | LC-MS: (M + H)+ found 588.1 |
| 1135 | LC-MS: (M + H)+ found 462.4 |
| 1137 | LC-MS: (M + H)+ found 438.2 |
| 1139 | LC-MS: (M + H)+ found 464.1 |
| 1142 | LC-MS: (M + H)+ found 498.1 |
| 1146 | LC-MS: (M + H)+ found 499.1 |
| 1148 | LC-MS: (M + H)+ found 465.1 |
| 1150 | LC-MS: (M + H)+ found 511.2 |
| 1153 | LC-MS: (M + H)+ found 454.1 |
| 1156 | LC-MS: (M + H)+ found 441.1 |
| 1157 | LC-MS: (M + H)+ found 449.1 |
| 1160 | LC-MS: (M + H)+ found 466.1 |
| 1162 | LC-MS: (M + H)+ found 735.4 |
| 1165 | LC-MS: (M + H)+ found 463.1 |
| 1167 | LC-MS: (M + H)+ found 483.1 |
| 1169 | LC-MS: (M + H)+ found 472.2 |
| 1171 | LC-MS: (M + H)+ found 514.4 |
| 1173 | LC-MS: (M + H)+ found 414.1 |
| 1176 | LC-MS: (M + H)+ found 421.1 |
| 1178 | LC-MS: (M + H)+ found 457.1 |
| 1182 | LC-MS: (M + H)+ found 473.2 |
| 1186 | LC-MS: (M + H)+ found 402.1 |
| 1187 | LC-MS: (M + H)+ found 439.2 |
| 1191 | LC-MS: (M + H)+ found 439.1 |
| 1193 | LC-MS: (M + H)+ found 633.2 |
| 1197 | LC-MS: (M + H)+ found 450.1 |
| 1200 | LC-MS: (M + H)+ found 456.2 |
| 1203 | LC-MS: (M + H)+ found 430.1 |
| 1207 | LC-MS: (M + H)+ found 466.2 |
| 1210 | LC-MS: (M + H)+ found 456.2 |
| 1211 | LC-MS: (M + H)+ found 489.2 |
| 1221 | LC-MS: (M + H)+ found 473.1 |
| 1223 | LC-MS: (M + H)+ found 433.1 |
| 1225 | LC-MS: (M + H)+ found 402.1 |
| 1226 | LC-MS: (M + H)+ found 504.1 |
| 1229 | LC-MS: (M + H)+ found 504.2 |
| 1231 | LC-MS: (M + H)+ found 458.1 |
| 1232 | LC-MS: (M + H)+ found 441.2 |
| 1238 | LC-MS: (M + H)+ found 450.2 |
| 1241 | LC-MS: (M + H)+ found 456.1 |
| 1243 | LC-MS: (M + H)+ found 487.1 |
| 1246 | LC-MS: (M + H)+ found 467.1 |
| 1248 | LC-MS: (M + H)+ found 467.1 |
| 1253 | LC-MS: (M + H)+ found 462.1 |
| 1254 | LC-MS: (M + H)+ found 438.1 |
| 1255 | LC-MS: (M + H)+ found 438.1 |
| 1260 | LC-MS: (M + H)+ found 443.1 |
| 1261 | LC-MS: (M + H)+ found 476.1 |
| 1263 | LC-MS: (M + H)+ found 516.2 |
| 1264 | LC-MS: (M + H)+ found 513.2 |
| 1266 | LC-MS: (M + H)+ found 526.2 |
| 1268 | LC-MS: (M + H)+ found 550.2 |
| 1270 | LC-MS: (M + H)+ found 597.3 |
| 1272 | LC-MS: (M + H)+ found 500.2 |
| 1273 | LC-MS: (M + H)+ found 508.1 |
| 1274 | LC-MS: (M + H)+ found 516.1 |
| 1279 | LC-MS: (M + H)+ found 422.3 |
| 1280 | LC-MS: (M + H)+ found 422.3 |
| 1284 | LC-MS: (M + H)+ found 455.1 |
| 1285 | LC-MS: (M + H)+ found 457.1 |
| 1288 | LC-MS: (M + H)+ found 520.1 |
| 1291 | LC-MS: (M + H)+ found 509.1 |
| 1295 | LC-MS: (M + H)+ found 534.2 |
| 1296 | LC-MS: (M + H)+ found 500.1 |
| 1298 | LC-MS: (M + H)+ found 454.1 |
| 1300 | LC-MS: (M + H)+ found 583.1 |
| 1301 | LC-MS: (M + H)+ found 566.1 |
| 1306 | LC-MS: (M + H)+ found 567.1 |
| 1312 | LC-MS: (M + H)+ found 520.1 |
| 1313 | LC-MS: (M + H)+ found 597.1 |
| 1314 | LC-MS: (M + H)+ found 597.2 |
| 1316 | LC-MS: (M + H)+ found 572.3 |
| 1317 | LC-MS: (M + H)+ found 483.1 |
| 1323 | LC-MS: (M + H)+ found 491.1 |
| 1326 | LC-MS: (M + H)+ found 446.1 |
| 1332 | LC-MS: (M + H)+ found 485.3 |
| 1333 | LC-MS: (M + H)+ found 572.1 |
| 1335 | LC-MS: (M + H)+ found 566.1 |
| 1348 | LC-MS: (M + H)+ found 446.2 |
| 1352 | LC-MS: (M + H)+ found 467.1 |
| 1353 | LC-MS: (M + H)+ found 433.1 |
| 1355 | LC-MS: (M + H)+ found 547.2 |
| 1358 | LC-MS: (M + H)+ found 467.2 |
| 1362 | LC-MS: (M + H)+ found 433.1 |
| 1369 | LC-MS: (M + H)+ found 476.1 |
| 1371 | LC-MS: (M + H)+ found 438.2 |
| 1374 | LC-MS: (M + H)+ found 445.1 |
| 1388 | LC-MS: (M + H)+ found 466.4 |
| 1393 | LC-MS: (M + H)+ found 509.1 |
| 1399 | LC-MS: (M + H)+ found 460.1 |
| 1402 | LC-MS: (M + H)+ found 428.1 |
| 1406 | LC-MS: (M + H)+ found 455.1 |
| 1407 | LC-MS: (M + H)+ found 456.1 |

### Assay

### PI3Kα Cellular Assay Experimental Procedure:

SKBR3 or T47D cells are seeded in DMEM containing 10% FBS at 25k cells/well into 96-well cell culture format. Cells are incubated overnight at 37° C. in a 5% CO2 incubator and the following day cell media is aspirated, adherent cells are washed 1× with room temperature PBS prior to serum-free media application. Cells are returned to 37° C. 5% CO2 incubator and incubated a further 16 hrs. Compounds are added to serum starved adherent cells with a top dose of 10,000 nM and 3× multiple dose reductions for a minimum dose of 0.5 nM diluted in DMSO. Cell/compound incubation continues for 1 hr in a 37° C., 5% CO2 incubator prior to 10 minute PIK3CA stimulation with 20 ng/ml EGF. Cells treated with 0.1% DMSO and 20 ng/mL EGF are employed as negative control, cells treated with 10 uM Alpelisib and 20 ng/mL EGF are employed as a positive control. After 10 mins, plates are removed from incubator and cells are lysed with buffer and shaking 45 minutes. 20 μL of lysate is transferred to an opti-384 plate and 2.5 μl of Phospho-AKT d₂ antibody with 2.5 μl of Phospho-AKT Eu Cryptate solution in the detection buffer are added to each well. 384 well plate is left overnight at room temperature before reading HTRF on an Envision plate reader.

The biological activity of certain compounds using the assays described above is shown in Table A. The ranges are as follows:
for T47D pAKT IC₅₀ (nM): A denotes <100 nM; B denotes 100 nM≤IC₅₀<500 nM; C denotes 500 nM≤IC₅₀<2,000 nM; and D denotes ≥2,000 nM. ND denotes value not determined with that assay for the specified compound;
for T47D (H1047R) selectivity over SKBR3 (WT): A denotes >20-fold; B denotes 20-fold≥value>5-fold; C denotes ≤5-fold. ND denotes value not determined with that assay for the specified compound; and
for T47D pAKT lipophilic efficiency (LiPE): A denotes >4; B denotes 4≥value>2; C denotes ≤2. ND denotes value not determined with that assay for the specified compound.

TABLE 1

| | | T47D pAKT S473 HTRF | |
|---|---|---|---|
| | T47D pAKT S473 | IC₅₀ (H1047R): T47D | |
| Cmpd | HTRF IC₅₀ (H1047R): | (H1047R) selectivity | T47D pAKT S473 HTRF |
| No. | Average IC₅₀ (nM) | over SKBR3 (WT) | IC₅₀ (H1047R): LipE |
| 1 | C | B | B |
| 2 | D | C | A |
| 7 | B | A | A |
| 8 | D | C | B |
| 12 | D | C | B |
| 29 | D | C | C |
| 41 | D | C | C |
| 42 | D | C | C |
| 44 | D | C | C |
| 47 | D | C | C |
| 124 | D | C | A |
| 126 | C | B | B |
| 132 | C | C | A |
| 134 | C | B | A |
| 137 | D | C | A |
| 138 | D | C | A |
| 139 | D | C | A |
| 140 | B | A | A |
| 144 | D | C | A |
| 145 | C | B | A |
| 146 | D | C | B |
| 147 | C | B | B |
| 149 | D | C | A |
| 150 | B | A | A |
| 151 | D | C | B |
| 152 | C | B | A |
| 154 | D | C | C |
| 155 | D | C | A |
| 156 | D | C | A |
| 157 | D | C | A |
| 158 | D | C | A |
| 159 | B | B | A |
| 160 | D | C | B |
| 178 | D | C | A |
| 179 | D | C | A |
| 194 | D | C | A |
| 226 | C | B | A |
| 227 | D | C | A |
| 229 | D | C | B |
| 230 | D | C | A |
| 231 | B | A | A |
| 232 | C | B | A |
| 233 | D | C | A |
| 234 | D | C | A |
| 237 | D | C | A |
| 239 | D | C | B |
| 240 | A | A | A |
| 243 | D | C | B |
| 245 | D | C | A |
| 248 | A | C | C |
| 249 | C | C | B |
| 252 | D | C | C |
| 258 | D | C | C |
| 263 | C | C | B |
| 264 | D | C | C |
| 265 | D | C | C |
| 266 | D | C | C |
| 267 | B | A | A |
| 268 | D | C | A |
| 269 | A | A | A |
| 271 | B | B | A |
| 272 | D | C | A |
| 273 | D | C | A |
| 276 | A | C | A |
| 280 | D | C | C |
| 281 | D | C | C |
| 284 | D | C | B |
| 285 | D | C | B |
| 286 | D | C | B |
| 287 | D | C | A |
| 288 | C | B | A |
| 289 | B | C | A |
| 290 | D | C | A |
| 292 | D | C | A |
| 293 | D | C | B |
| 294 | C | C | A |
| 295 | C | B | A |
| 296 | D | C | B |
| 297 | B | A | A |
| 299 | D | C | A |
| 300 | D | C | B |
| 302 | C | B | A |
| 303 | D | C | A |
| 304 | D | C | B |
| 307 | C | B | A |
| 308 | D | C | A |
| 309 | D | C | A |
| 310 | D | C | A |
| 311 | D | C | A |
| 313 | D | C | A |
| 315 | C | B | A |
| 316 | D | C | B |
| 317 | B | A | A |
| 318 | D | C | B |
| 319 | B | A | A |
| 320 | D | C | B |
| 321 | D | C | B |
| 322 | D | C | A |
| 323 | D | C | A |
| 325 | D | C | A |
| 326 | D | C | A |
| 328 | D | C | B |
| 329 | D | C | A |
| 330 | D | C | A |
| 331 | D | C | A |
| 332 | D | C | A |
| 333 | D | C | A |
| 334 | D | C | A |
| 335 | D | C | A |
| 336 | D | C | A |
| 337 | D | C | C |
| 338 | D | C | B |
| 339 | D | C | B |
| 342 | D | C | B |
| 343 | D | C | B |
| 344 | D | C | B |
| 346 | C | A | A |
| 347 | B | A | A |
| 348 | B | A | A |
| 349 | B | A | A |
| 350 | D | C | A |
| 351 | D | C | B |
| 352 | D | C | B |
| 353 | D | C | B |
| 354 | D | C | A |
| 355 | C | B | A |
| 356 | D | C | B |
| 357 | D | C | A |
| 358 | D | C | A |
| 359 | A | A | A |
| 360 | D | C | B |
| 361 | A | A | A |
| 362 | D | C | B |
| 363 | B | A | A |
| 364 | A | A | A |
| 365 | D | C | B |
| 368 | D | C | A |
| 369 | D | C | A |
| 372 | B | B | A |
| 373 | D | C | B |
| 374 | A | A | A |
| 375 | D | C | A |
| 376 | D | C | A |
| 377 | D | C | A |
| 378 | D | C | A |
| 379 | D | C | A |
| 380 | D | C | A |
| 381 | A | A | A |
| 382 | D | C | B |
| 383 | C | B | A |
| 384 | B | A | A |
| 385 | C | B | A |
| 386 | D | C | B |
| 389 | D | C | A |
| 390 | D | C | A |
| 391 | D | C | A |
| 392 | D | C | B |
| 393 | A | A | A |
| 394 | D | C | A |
| 395 | D | C | A |
| 396 | D | C | A |
| 397 | C | B | A |
| 398 | D | C | A |
| 399 | C | B | A |
| 400 | D | C | B |
| 401 | D | C | A |
| 402 | D | C | A |
| 403 | D | C | A |
| 404 | D | C | A |
| 405 | D | C | A |
| 406 | D | C | A |
| 407 | A | C | A |
| 408 | D | C | A |
| 409 | D | C | A |
| 410 | C | B | A |
| 411 | D | C | A |
| 412 | C | B | A |
| 413 | C | B | A |
| 414 | B | A | A |
| 415 | D | C | A |
| 416 | B | A | A |
| 417 | B | A | A |
| 418 | B | A | A |
| 419 | D | C | B |
| 420 | B | A | A |
| 421 | D | C | B |
| 422 | A | A | A |
| 423 | D | C | A |
| 424 | A | A | A |
| 425 | D | C | B |
| 426 | B | A | A |
| 427 | D | C | B |
| 428 | D | C | A |
| 429 | D | C | A |
| 430 | D | C | A |
| 431 | A | A | A |
| 432 | D | C | B |
| 433 | D | C | B |
| 434 | D | C | B |
| 435 | B | A | A |
| 436 | D | C | B |
| 437 | D | C | A |
| 438 | D | C | B |
| 439 | D | C | B |
| 440 | C | B | B |
| 441 | D | C | A |
| 442 | D | C | B |
| 444 | D | C | A |
| 446 | D | C | A |
| 447 | D | C | A |
| 448 | D | C | A |
| 449 | D | C | A |
| 450 | D | C | A |
| 451 | D | C | A |
| 454 | D | C | A |
| 455 | D | C | A |
| 456 | D | C | B |
| 457 | A | A | A |
| 458 | D | C | A |
| 459 | D | C | A |
| 460 | C | B | A |
| 461 | D | C | A |
| 462 | D | C | A |
| 463 | C | B | A |
| 464 | D | C | B |
| 465 | C | B | A |
| 466 | D | C | A |
| 467 | D | C | B |
| 468 | D | C | B |
| 469 | D | C | B |
| 470 | D | C | A |
| 471 | C | B | A |
| 472 | C | B | A |
| 473 | D | C | A |
| 474 | C | B | B |
| 475 | D | C | A |
| 476 | D | C | B |
| 478 | B | A | A |
| 480 | C | B | A |
| 481 | D | C | A |
| 482 | C | B | A |
| 483 | C | B | A |
| 484 | D | C | A |
| 485 | D | C | A |
| 486 | B | A | A |
| 487 | D | C | A |
| 488 | D | C | A |
| 489 | D | C | A |
| 490 | D | C | A |
| 491 | D | C | A |
| 494 | D | C | B |
| 495 | D | C | B |
| 496 | C | C | B |
| 497 | C | B | A |
| 498 | D | C | B |
| 499 | B | A | A |
| 500 | D | C | B |
| 501 | D | C | A |
| 502 | D | C | A |
| 503 | D | C | A |
| 504 | D | C | A |
| 505 | D | C | B |
| 506 | C | B | A |
| 507 | D | C | A |
| 508 | D | C | A |
| 509 | D | C | A |
| 510 | D | C | A |
| 511 | D | C | B |
| 512 | A | A | A |
| 513 | D | C | B |
| 514 | C | B | A |
| 515 | C | B | A |
| 516 | B | A | A |
| 517 | D | C | B |
| 518 | D | C | A |
| 519 | D | C | B |
| 520 | D | C | B |
| 521 | D | C | B |
| 522 | D | C | B |
| 523 | C | B | A |
| 524 | D | C | B |
| 525 | D | C | B |
| 526 | D | C | A |
| 527 | D | C | A |
| 528 | D | C | A |
| 529 | D | C | A |
| 530 | D | C | A |
| 531 | D | C | A |
| 532 | D | C | A |
| 533 | D | C | A |
| 534 | D | C | B |
| 535 | D | C | B |
| 536 | A | A | A |
| 537 | C | B | A |
| 538 | D | C | B |
| 539 | C | B | A |
| 540 | D | C | B |
| 541 | B | A | A |
| 542 | D | C | B |
| 543 | D | C | B |
| 544 | D | C | B |
| 545 | B | A | A |
| 546 | D | C | B |
| 547 | D | C | B |
| 548 | D | C | B |
| 549 | D | C | A |
| 550 | D | C | A |
| 551 | D | C | A |
| 552 | D | C | A |
| 553 | D | C | A |
| 554 | D | C | A |
| 555 | D | C | A |
| 556 | D | C | A |
| 557 | D | C | A |
| 558 | B | A | B |
| 559 | D | C | A |
| 560 | C | B | B |
| 561 | D | C | B |
| 562 | D | C | B |
| 563 | D | C | B |
| 564 | D | C | B |
| 565 | D | C | B |
| 566 | D | C | B |
| 570 | B | A | A |
| 571 | D | C | B |
| 572 | B | A | A |
| 573 | D | C | B |
| 574 | D | C | B |
| 575 | B | A | A |
| 576 | D | C | B |
| 577 | B | A | A |
| 578 | D | C | A |
| 579 | D | C | A |
| 580 | D | C | A |
| 581 | D | C | A |
| 582 | D | C | B |
| 583 | A | A | A |
| 584 | D | C | C |
| 585 | D | C | C |
| 586 | D | C | B |
| 587 | D | C | C |
| 590 | B | A | A |
| 591 | D | C | A |
| 592 | D | C | B |
| 593 | A | A | A |
| 594 | D | C | B |
| 595 | B | A | A |
| 596 | D | C | B |
| 597 | D | C | A |
| 598 | D | C | A |
| 599 | C | B | A |
| 600 | C | B | A |
| 601 | D | C | A |
| 602 | D | C | B |
| 603 | D | C | B |
| 604 | D | C | B |
| 605 | A | A | B |
| 606 | D | C | C |
| 607 | D | C | B |
| 608 | D | C | B |
| 609 | D | C | B |
| 610 | D | C | B |
| 611 | A | A | A |
| 612 | D | C | B |
| 613 | D | C | A |
| 614 | B | A | B |
| 615 | D | C | C |
| 616 | D | C | A |
| 617 | D | C | A |
| 618 | D | C | A |
| 619 | D | C | A |
| 620 | A | A | B |
| 621 | D | C | C |
| 622 | B | A | B |
| 623 | A | A | B |
| 624 | D | C | C |
| 625 | B | A | B |
| 626 | B | A | A |
| 627 | D | C | B |
| 628 | D | C | B |
| 629 | A | A | A |
| 630 | D | C | A |
| 631 | D | C | A |
| 632 | B | A | A |
| 633 | D | C | B |
| 634 | D | C | B |
| 635 | C | B | A |
| 636 | C | B | A |
| 637 | B | A | A |
| 638 | D | C | B |
| 639 | C | B | A |
| 641 | D | C | B |
| 644 | D | C | B |
| 645 | D | C | B |
| 646 | D | C | B |
| 647 | D | C | C |
| 648 | D | C | A |
| 649 | D | C | A |
| 650 | D | C | A |
| 651 | D | C | A |
| 652 | A | A | A |
| 653 | A | A | A |
| 654 | C | B | B |
| 655 | D | C | B |
| 656 | D | C | A |
| 657 | C | B | A |
| 658 | B | A | A |
| 659 | D | C | A |
| 660 | A | A | A |
| 661 | D | C | B |
| 662 | B | A | A |
| 663 | D | C | B |
| 664 | B | A | A |
| 665 | B | A | C |
| 666 | D | C | B |
| 673 | C | B | A |
| 674 | D | C | A |
| 675 | D | C | B |
| 676 | D | C | B |
| 677 | D | C | B |
| 678 | D | C | B |
| 679 | D | C | B |
| 680 | C | C | B |
| 681 | D | C | C |
| 682 | D | C | C |
| 683 | D | C | C |
| 684 | D | C | B |
| 685 | D | C | B |
| 686 | D | C | B |
| 687 | D | C | B |
| 688 | D | C | B |
| 689 | D | C | A |
| 690 | D | C | A |
| 691 | A | A | A |
| 692 | B | A | B |
| 693 | B | A | A |
| 694 | D | C | B |
| 695 | B | A | A |
| 696 | A | A | A |
| 697 | D | C | B |
| 698 | C | C | B |
| 699 | D | C | C |
| 700 | B | A | B |
| 701 | D | C | B |
| 702 | C | B | B |
| 703 | D | C | B |
| 704 | D | C | B |
| 705 | D | C | B |
| 706 | C | B | B |
| 707 | D | C | A |
| 708 | D | C | A |
| 709 | D | C | A |
| 710 | D | C | A |
| 711 | A | A | A |
| 712 | D | C | B |
| 713 | C | C | B |
| 714 | D | C | B |
| 715 | D | C | A |
| 716 | D | C | A |
| 717 | D | C | A |
| 718 | D | C | C |
| 719 | B | A | B |
| 720 | C | B | B |
| 721 | B | A | A |
| 722 | A | A | A |
| 723 | D | C | B |
| 724 | B | A | B |
| 725 | A | A | A |
| 726 | D | B | A |
| 727 | D | C | C |
| 728 | D | C | C |
| 729 | B | A | A |
| 730 | A | A | A |
| 731 | D | C | B |
| 732 | D | C | B |
| 733 | D | C | B |
| 734 | D | C | A |
| 735 | D | C | B |
| 736 | B | A | A |
| 737 | B | A | B |
| 738 | D | C | C |
| 739 | D | C | B |
| 740 | A | A | A |
| 741 | D | C | A |
| 742 | D | C | C |
| 743 | B | A | B |
| 744 | B | B | B |
| 745 | D | C | B |
| 746 | D | C | C |
| 747 | C | B | B |
| 748 | A | A | A |
| 749 | D | C | B |
| 750 | D | C | A |
| 751 | B | A | A |
| 752 | D | C | B |
| 753 | C | B | A |
| 754 | D | C | A |
| 755 | D | C | B |
| 756 | D | C | B |
| 757 | D | C | A |
| 758 | B | A | A |
| 759 | D | C | A |
| 760 | A | A | A |
| 761 | D | C | B |
| 762 | A | A | A |
| 763 | C | B | B |
| 764 | B | A | A |
| 765 | B | A | A |
| 766 | D | C | B |
| 767 | A | A | A |
| 768 | B | A | A |
| 769 | B | A | B |
| 770 | C | B | A |
| 771 | C | B | A |
| 772 | D | C | B |
| 773 | C | B | A |
| 774 | D | C | A |
| 775 | A | A | A |
| 776 | D | C | B |
| 777 | B | A | B |
| 778 | D | C | B |
| 779 | D | C | B |
| 780 | D | C | B |
| 781 | B | A | A |
| 782 | D | C | B |
| 783 | D | C | A |
| 784 | D | C | A |
| 785 | A | A | A |
| 786 | D | C | B |
| 787 | D | C | C |
| 788 | B | A | B |
| 789 | D | C | A |
| 790 | D | C | A |
| 791 | D | C | A |
| 792 | D | C | A |
| 793 | D | C | B |
| 794 | B | A | A |
| 795 | D | C | B |
| 796 | D | C | B |
| 797 | D | C | B |
| 798 | A | A | A |
| 799 | D | C | A |
| 801 | D | C | B |
| 802 | D | C | C |
| 803 | D | C | C |
| 804 | A | A | A |
| 805 | D | C | B |
| 806 | D | C | B |
| 807 | A | A | A |
| 808 | D | C | B |
| 809 | A | A | A |
| 810 | D | C | B |
| 811 | C | B | B |
| 812 | D | C | B |
| 813 | D | C | B |
| 814 | D | C | B |
| 815 | D | C | B |
| 816 | D | C | B |
| 817 | D | C | B |
| 818 | A | A | A |
| 819 | A | A | A |
| 820 | D | C | B |
| 821 | B | A | B |
| 822 | A | A | A |
| 823 | D | C | C |
| 824 | D | C | A |
| 825 | D | C | A |
| 826 | C | B | A |
| 827 | D | C | B |
| 828 | C | B | B |
| 829 | D | C | B |
| 830 | B | A | A |
| 831 | B | A | A |
| 832 | C | B | C |
| 833 | B | A | B |
| 834 | D | C | C |
| 835 | B | A | C |
| 836 | D | C | C |
| 840 | D | C | B |
| 841 | D | C | B |
| 842 | B | A | B |
| 843 | C | B | A |
| 844 | C | B | A |
| 845 | D | C | C |
| 846 | D | C | B |
| 847 | D | C | C |
| 848 | A | A | B |
| 849 | B | A | B |
| 850 | D | C | B |
| 851 | A | A | A |
| 852 | D | C | B |
| 853 | A | A | B |
| 854 | D | C | C |
| 855 | B | A | B |
| 856 | A | A | B |
| 857 | D | C | C |
| 858 | B | A | A |
| 859 | B | A | B |
| 860 | C | B | A |
| 861 | D | C | A |
| 862 | D | C | A |
| 863 | D | C | C |
| 865 | D | C | C |
| 868 | C | B | B |
| 869 | B | A | A |
| 870 | B | A | A |
| 871 | D | C | B |
| 872 | D | C | A |
| 873 | D | C | A |
| 874 | D | C | C |
| 875 | A | A | C |
| 876 | D | C | B |
| 877 | B | A | B |
| 878 | D | C | C |
| 879 | D | C | B |
| 880 | C | B | A |
| 881 | B | A | A |
| 882 | D | C | B |
| 883 | B | A | C |
| 884 | D | C | C |
| 885 | D | C | A |
| 886 | D | C | B |
| 887 | D | C | A |
| 888 | D | C | A |
| 889 | B | A | C |
| 890 | A | A | A |
| 891 | D | C | B |
| 892 | A | A | A |
| 893 | D | C | B |
| 894 | B | A | B |
| 895 | C | B | A |
| 896 | B | A | A |
| 897 | D | C | A |
| 898 | B | A | A |
| 899 | A | A | A |
| 900 | C | C | B |
| 901 | B | A | B |
| 902 | A | A | A |
| 903 | C | B | B |
| 904 | B | A | A |
| 905 | D | C | A |
| 906 | D | C | A |
| 907 | D | C | B |
| 908 | B | A | A |
| 909 | D | C | B |
| 910 | D | C | B |
| 911 | D | C | B |
| 912 | D | C | B |
| 913 | D | C | B |
| 914 | D | C | B |
| 915 | B | A | A |
| 916 | B | A | A |
| 917 | D | C | B |
| 918 | D | C | B |
| 919 | A | A | A |
| 920 | D | C | B |
| 921 | D | C | B |
| 922 | A | A | A |
| 923 | C | B | A |
| 924 | B | A | A |
| 925 | C | B | B |
| 926 | D | C | C |
| 927 | A | A | A |
| 928 | D | C | B |
| 929 | D | C | B |
| 930 | D | C | C |
| 931 | A | A | A |
| 932 | D | C | C |
| 933 | D | C | B |
| 934 | B | A | A |
| 935 | D | C | B |
| 936 | D | C | B |
| 937 | A | A | A |
| 938 | A | A | A |
| 939 | D | C | B |
| 940 | D | C | B |
| 941 | D | C | B |
| 942 | D | C | C |
| 943 | B | A | B |
| 944 | B | A | C |
| 945 | A | A | A |
| 946 | C | B | B |
| 947 | A | A | A |
| 948 | C | B | B |
| 949 | A | A | A |
| 950 | D | C | B |
| 951 | A | A | A |
| 952 | C | B | B |
| 953 | A | A | A |
| 954 | C | B | B |
| 955 | D | C | A |
| 956 | D | C | A |
| 957 | D | C | C |
| 958 | D | C | C |
| 959 | A | A | A |
| 960 | D | C | B |
| 961 | B | A | B |
| 962 | D | C | C |
| 963 | A | A | A |
| 964 | C | B | B |
| 965 | A | A | A |
| 966 | D | C | C |
| 967 | A | A | A |
| 968 | B | A | A |
| 969 | A | A | A |
| 970 | B | A | B |
| 971 | A | A | A |
| 972 | A | A | B |
| 973 | A | A | A |
| 974 | C | B | B |
| 975 | A | A | A |
| 976 | D | C | B |
| 977 | D | C | B |
| 978 | A | A | A |
| 979 | D | C | C |
| 980 | D | C | A |
| 981 | D | C | B |
| 982 | A | A | B |
| 983 | C | B | B |
| 984 | A | A | A |
| 985 | D | C | C |
| 986 | B | B | B |
| 987 | B | A | B |
| 988 | D | C | B |
| 989 | B | A | C |
| 990 | B | A | B |
| 991 | B | A | B |
| 992 | D | C | C |
| 993 | D | C | C |
| 994 | B | A | C |
| 995 | D | C | C |
| 996 | B | A | B |
| 997 | B | A | B |
| 998 | D | C | C |
| 999 | B | A | B |
| 1000 | C | B | B |
| 1001 | D | C | A |
| 1002 | C | A | C |
| 1003 | D | C | C |
| 1004 | D | C | A |
| 1005 | C | B | B |
| 1006 | D | C | C |
| 1007 | D | C | C |
| 1008 | A | A | B |
| 1009 | D | C | C |
| 1010 | C | B | B |
| 1011 | D | C | C |
| 1012 | C | B | C |
| 1013 | C | B | B |
| 1014 | D | C | C |
| 1015 | A | A | C |
| 1016 | D | C | A |
| 1017 | D | C | A |
| 1018 | D | C | B |
| 1019 | B | A | B |
| 1020 | D | C | B |
| 1021 | A | A | A |
| 1022 | A | A | A |
| 1023 | D | C | C |
| 1024 | D | C | C |
| 1025 | C | B | B |
| 1026 | B | A | B |
| 1027 | B | A | B |
| 1028 | D | C | C |
| 1029 | D | C | C |
| 1030 | B | A | B |
| 1031 | B | A | B |
| 1032 | D | C | C |
| 1033 | B | A | B |
| 1034 | A | A | B |
| 1035 | D | C | C |
| 1036 | A | A | A |
| 1037 | D | C | B |
| 1038 | A | A | A |
| 1039 | D | C | C |
| 1040 | D | C | B |
| 1041 | D | C | B |
| 1042 | A | A | B |
| 1043 | C | B | B |
| 1044 | D | C | B |
| 1045 | C | B | B |
| 1046 | B | A | C |
| 1047 | B | A | C |
| 1048 | B | A | B |
| 1049 | D | C | B |
| 1050 | A | A | A |
| 1051 | D | C | B |
| 1052 | B | A | B |
| 1053 | D | C | C |
| 1054 | C | B | C |
| 1055 | B | A | A |
| 1056 | D | C | B |
| 1057 | D | C | B |
| 1058 | A | A | A |
| 1059 | B | A | B |
| 1060 | D | C | C |
| 1061 | D | C | C |
| 1062 | D | C | C |
| 1063 | B | A | B |
| 1064 | B | A | B |
| 1065 | D | C | C |
| 1066 | B | A | B |
| 1067 | A | A | A |
| 1068 | D | C | B |
| 1069 | A | A | A |
| 1070 | A | A | A |
| 1071 | D | C | B |
| 1072 | B | A | B |
| 1073 | D | C | B |
| 1074 | A | A | A |
| 1075 | D | C | B |
| 1077 | C | B | A |
| 1078 | D | C | B |
| 1079 | B | A | C |
| 1080 | D | C | C |
| 1081 | A | A | A |
| 1082 | C | B | B |
| 1083 | B | A | B |
| 1084 | D | C | C |
| 1085 | D | C | C |
| 1086 | D | C | C |
| 1087 | D | C | C |
| 1088 | D | C | C |
| 1089 | D | C | C |
| 1090 | A | A | B |
| 1091 | B | A | C |
| 1092 | B | A | B |
| 1093 | D | C | C |
| 1094 | B | A | C |
| 1095 | D | C | C |
| 1096 | A | A | B |
| 1097 | A | A | B |
| 1098 | D | C | C |
| 1099 | A | A | A |
| 1100 | D | C | B |
| 1101 | A | A | A |
| 1102 | D | C | B |
| 1103 | A | A | A |
| 1104 | B | A | A |
| 1105 | A | A | A |
| 1106 | B | A | B |
| 1107 | A | A | A |
| 1108 | C | B | B |
| 1109 | A | A | B |
| 1110 | D | C | C |
| 1111 | A | A | A |
| 1112 | C | B | A |
| 1113 | D | C | C |
| 1114 | A | A | B |
| 1115 | A | A | A |
| 1116 | C | B | B |
| 1117 | C | B | C |
| 1118 | B | A | A |
| 1119 | C | B | B |
| 1121 | D | C | C |
| 1122 | D | C | C |
| 1123 | D | C | C |
| 1124 | D | C | C |
| 1125 | B | A | C |
| 1126 | D | C | C |
| 1127 | D | C | C |
| 1128 | D | C | C |
| 1129 | C | B | C |
| 1130 | C | B | C |
| 1131 | D | C | C |
| 1132 | D | C | C |
| 1133 | D | C | C |
| 1134 | C | B | C |
| 1135 | B | A | B |
| 1136 | D | C | C |
| 1137 | A | A | B |
| 1138 | D | C | C |
| 1139 | B | A | C |
| 1140 | B | A | C |
| 1141 | D | C | C |
| 1142 | B | A | B |
| 1143 | B | A | B |
| 1144 | D | C | C |
| 1145 | B | A | B |
| 1146 | A | A | A |
| 1147 | D | C | B |
| 1148 | A | A | A |
| 1149 | D | C | B |
| 1150 | A | A | A |
| 1151 | D | C | C |
| 1152 | B | A | B |
| 1153 | B | A | B |
| 1154 | D | C | C |
| 1155 | D | C | B |
| 1156 | A | A | A |
| 1157 | B | A | A |
| 1158 | D | C | B |
| 1159 | D | C | B |
| 1160 | A | A | A |
| 1161 | D | C | B |
| 1162 | B | A | B |
| 1163 | B | A | C |
| 1164 | D | C | C |
| 1165 | B | A | C |
| 1166 | D | C | C |
| 1167 | A | A | A |
| 1168 | D | C | B |
| 1169 | A | A | A |
| 1170 | B | A | B |
| 1171 | A | A | B |
| 1172 | D | C | C |
| 1173 | A | A | B |
| 1174 | D | C | C |
| 1175 | D | C | C |
| 1176 | A | A | B |
| 1177 | B | A | A |
| 1178 | A | A | A |
| 1179 | C | B | B |
| 1180 | D | C | C |
| 1181 | D | C | C |
| 1182 | A | A | A |
| 1183 | C | B | B |
| 1184 | B | A | A |
| 1185 | C | B | B |
| 1186 | B | A | B |
| 1187 | A | A | A |
| 1188 | C | B | B |
| 1189 | D | C | B |
| 1190 | D | C | B |
| 1191 | A | A | A |
| 1192 | D | C | B |
| 1193 | A | A | B |
| 1194 | D | C | C |
| 1195 | C | B | C |
| 1196 | D | C | C |
| 1197 | A | A | A |
| 1198 | D | C | B |
| 1199 | B | A | A |
| 1200 | A | A | A |
| 1201 | A | C | B |
| 1202 | B | A | A |
| 1203 | B | A | A |
| 1204 | D | C | B |
| 1205 | D | C | B |
| 1206 | C | B | B |
| 1207 | A | A | A |
| 1208 | B | A | B |
| 1209 | D | C | C |
| 1200 | A | A | B |
| 1211 | A | A | A |
| 1212 | D | C | B |
| 1213 | D | C | C |
| 1214 | D | C | C |
| 1215 | D | C | C |
| 1216 | D | C | C |
| 1217 | D | C | C |
| 1218 | D | C | C |
| 1219 | D | C | C |
| 1220 | D | C | B |
| 1221 | A | A | A |
| 1222 | D | C | B |
| 1223 | A | A | A |
| 1224 | D | C | B |
| 1225 | B | A | B |
| 1226 | B | A | B |
| 1227 | D | C | C |
| 1228 | D | C | C |
| 1229 | B | A | B |
| 1230 | D | C | C |
| 1231 | A | A | A |
| 1232 | A | A | A |
| 1233 | D | C | B |
| 1234 | D | C | C |
| 1235 | B | A | C |
| 1236 | D | C | C |
| 1237 | D | C | C |
| 1238 | A | A | A |
| 1239 | D | C | B |
| 1240 | A | C | B |
| 1241 | D | A | A |
| 1242 | B | A | B |
| 1243 | A | A | B |
| 1244 | C | B | C |
| 1245 | B | A | B |
| 1246 | B | A | B |
| 1247 | D | C | C |
| 1248 | B | A | B |
| 1249 | B | A | C |
| 1250 | B | A | C |
| 1251 | D | C | C |
| 1252 | C | B | B |
| 1253 | B | A | A |
| 1254 | B | A | A |
| 1255 | A | A | A |
| 1259 | D | C | B |
| 1260 | A | A | A |
| 1261 | B | A | B |
| 1262 | D | C | B |
| 1263 | B | A | B |
| 1264 | A | A | A |
| 1265 | D | C | B |
| 1266 | A | A | B |
| 1267 | D | C | C |
| 1268 | A | A | B |
| 1269 | D | C | C |
| 1270 | A | A | A |
| 1271 | C | B | B |
| 1272 | B | A | A |
| 1273 | B | A | A |
| 1274 | B | A | B |
| 1275 | D | C | C |
| 1276 | A | C | C |
| 1277 | B | A | B |
| 1278 | D | C | C |
| 1279 | A | A | A |
| 1280 | A | A | A |
| 1281 | C | B | B |
| 1282 | D | C | B |
| 1283 | D | C | C |
| 1284 | A | A | A |
| 1285 | A | A | A |
| 1286 | C | B | B |
| 1287 | D | C | C |
| 1288 | A | A | B |
| 1289 | B | A | A |
| 1290 | D | C | B |
| 1291 | B | A | A |
| 1292 | D | C | B |
| 1293 | B | A | A |
| 1294 | C | B | B |
| 1295 | A | A | B |
| 1296 | B | A | A |
| 1297 | D | C | B |
| 1298 | A | A | A |
| 1299 | D | C | B |
| 1300 | A | A | B |
| 1301 | B | A | B |
| 1302 | D | C | A |
| 1303 | D | C | A |
| 1304 | C | B | A |
| 1305 | D | C | B |
| 1306 | A | A | A |
| 1307 | C | B | B |
| 1308 | B | A | A |
| 1309 | C | B | B |
| 1310 | D | C | C |
| 1311 | D | C | C |
| 1312 | A | A | A |
| 1313 | B | A | B |
| 1314 | B | A | B |
| 1315 | D | C | C |
| 1316 | B | A | B |
| 1317 | A | A | B |
| 1318 | D | C | C |
| 1319 | D | C | B |
| 1320 | C | B | B |
| 1321 | C | B | B |
| 1322 | D | C | A |
| 1323 | A | A | B |
| 1324 | B | A | B |
| 1325 | D | C | B |
| 1326 | A | A | A |
| 1327 | C | B | B |
| 1328 | D | C | C |
| 1329 | B | A | A |
| 1330 | D | C | B |
| 1331 | D | C | B |
| 1332 | A | A | A |
| 1333 | A | A | A |
| 1334 | C | B | B |
| 1335 | A | A | A |
| 1336 | D | C | B |
| 1337 | D | C | B |
| 1338 | D | C | A |
| 1339 | D | C | A |
| 1340 | D | C | B |
| 1341 | C | B | B |
| 1342 | D | C | B |
| 1343 | D | C | A |
| 1344 | C | B | A |
| 1345 | D | C | B |
| 1346 | D | C | C |
| 1347 | C | B | B |
| 1348 | A | A | A |
| 1349 | D | C | B |
| 1350 | C | B | B |
| 1351 | D | C | B |
| 1352 | B | A | B |
| 1353 | B | A | A |
| 1354 | D | C | C |
| 1355 | A | A | A |
| 1356 | B | A | C |
| 1357 | D | C | B |
| 1358 | A | A | A |
| 1359 | D | C | B |
| 1360 | D | C | B |
| 1361 | B | A | A |
| 1362 | A | A | A |
| 1363 | D | C | B |
| 1364 | D | C | B |
| 1365 | B | A | B |
| 1366 | B | A | A |
| 1367 | B | A | C |
| 1368 | D | C | C |
| 1369 | B | A | B |
| 1370 | D | C | B |
| 1371 | A | A | A |
| 1372 | D | C | B |
| 1373 | D | C | B |
| 1374 | B | A | B |
| 1375 | D | C | C |
| 1376 | B | A | A |
| 1377 | D | C | B |
| 1378 | B | A | A |
| 1379 | C | B | B |
| 1380 | D | C | B |
| 1381 | D | C | B |
| 1382 | B | A | B |
| 1383 | B | A | A |
| 1384 | D | C | B |
| 1385 | D | C | B |
| 1386 | D | C | B |
| 1387 | D | C | C |
| 1388 | A | A | A |
| 1389 | C | B | B |
| 1390 | D | C | B |
| 1391 | D | C | C |
| 1392 | D | C | C |
| 1393 | A | A | B |
| 1394 | D | C | B |
| 1395 | D | C | C |
| 1396 | D | C | C |
| 1397 | D | C | C |
| 1398 | C | B | C |
| 1399 | B | A | B |
| 1400 | D | C | C |
| 1402 | B | A | B |
| 1403 | D | C | C |
| 1404 | D | C | C |
| 1405 | D | C | C |
| 1406 | A | A | B |
| 1407 | A | A | B |
| 1408 | A | A | A |
| 1409 | B | A | C |
| 1410 | A | A | A |
| 1411 | A | A | A |
| 1412 | A | A | B |
| 1413 | B | A | A |

What is claimed is:

1. A compound of Formula (I): 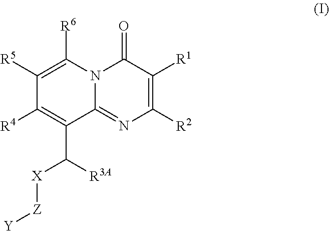
or a pharmaceutically acceptable salt thereof, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R² is phenyl optionally substituted with 1-3 independently selected R^{2A}, 5-10 membered heteroaryl optionally substituted with 1-3 independently selected R^{2A}, 4-10 membered heterocyclyl optionally substituted with 1-3 independently selected R^{2A}, 4-10 membered cycloalkyl optionally substituted with 1-3 independently selected R^{2A}, C1-C6 alkoxyalkyl optionally substituted with —C(═O)NR^{A}R^{C}, C1-C6 alkoxy optionally substituted with —C(═O)NR^{A}R^{C} or —O(R^{2B});
each R^{2A} is independently selected from:
(i) halogen,
(ii) cyano,
(iii) hydroxyl,
(iv) —NR^{A}R^{B},
(v) —C(═O)NR^{A}R^{B}, 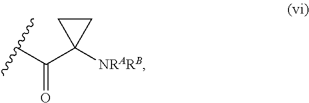
(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, —C(═O)NR^{A}R^{B}, —NHC(═O)C1-C6 alkyl optionally substituted with —NR^{A}R^{B} or C1-C6 alkyl optionally substituted with C1-C6 alkoxy or —NHC(═O)C1-C6 alkyl optionally substituted with C3-C16 cycloalkyl,
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
R^{2B} is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A};
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NIHR, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or 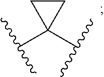 [SMILES: *C1(*)CC1]
Z is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(=NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y}1, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

2. The compound of claim 1, wherein Z is O.

3. The compound of claim 1, wherein Z is NR^{3B}.

4. The compound of any one of claims 1-3, wherein R² is phenyl optionally substituted with 1-3 independently selected R^{2A}.

5. The compound of any one of claims 1-3, wherein R² is 5-10 membered heteroaryl optionally substituted with 1-3 independently selected R^{2A}.

6. The compound of any one of claims 1-5, wherein R² is 4-10 membered heterocyclyl optionally substituted with 1-3 independently selected R^{2A}.

7. The compound of any one of claims 1-5, wherein R² is 4-10 membered cycloalkyl optionally substituted with 1-3 independently selected R^{2A} or —O(R^{2B}).

8. The compound of any one of claims 1-5, wherein R² is C1-C6 alkoxy substituted with —C(═O)NR^{A}R^{C}.

9. The compound of any one of claims 1-5, wherein R² is C1-C6 alkoxy.

10. The compound of any one of claims 1-5, R² is C1-C6 alkoxyalkyl optionally substituted with —C(═O)NR^{A}R^{C}.

11. The compound of any one of claims 1-5, R² is C1-C6 alkoxyalkyl.

12. The compound of any one of claims 1-11, wherein X is a bond.

13. The compound of any one of claims 1-11, wherein X is CH₂.

14. The compound of any one of claims 1-11, wherein X is CH(CH₃).

15. The compound of any one of claims 1-11, wherein X is C(CH₃)₂.

16. The compound ofany one of claims 1-11, wherein X is

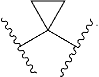 [SMILES: *C1(*)CC1]

17. The compound of any one of claims 1-16, wherein Y is phenyl optionally substituted with 1-3 independently selected R^{Y}.

18. The compound of any one of claims 1-16, wherein Y is naphthyl optionally substituted with 1-3 independently selected R^{Y}.

19. The compound of any one of claims 1-16, wherein Y is 5-10 membered heteroaryl optionally substituted with 1-3 independently selected R^{Y}.

20. The compound of any one of claims 1-19, wherein R^{Y} is halogen.

21. The compound of any one of claims 1-19, wherein R^{Y} is hydroxyl.

22. The compound of any one of claims 1-19, wherein R^{Y} is cyano.

23. The compound of any one of claims 1-19, wherein R^{Y} is C1-C6 haloalkyl.

24. The compound of any one of claims 1-19, wherein R^{Y} is C1-C6 alkoxy.

25. The compound of any one of claims 1-19, wherein R^{Y} is C1-C6 haloalkoxy.

26. The compound of any one of claims 1-19, wherein R^{Y} is C1-C6 hydroxyalkyl.

27. The compound of any one of claims 1-19, wherein R^{Y} is —NHC(═O)R^{C}.

28. The compound of any one of claims 1-19, wherein R^{Y} is —C(═O)NR^{Y1}.

29. The compound of any one of claims 1-19, wherein R^{Y} is —CO₂R^{A}.

30. The compound of any one of claims 1-19, wherein R^{Y} is —SO₂NR^{F}R^{G}.

31. The compound of any one of claims 1-19, wherein R^{Y} is —NHSO₂R^{F}.

32. The compound of any one of claims 1-19, wherein R^{Y} is —S(═O)(=NR^{F})R^{G}.

33. The compound of any one of claims 1-19, wherein R^{Y} is —SO₂(C1-C6 alkyl).

34. The compound of any one of claims 1-19, wherein R^{Y} is —C(═O)NR^{A}R^{B}.

35. The compound of any one of claims 1-19, wherein R^{Y} is 4-6 membered heteroaryl.

36. The compound of any one of claims 1-19, wherein R^{Y} is heteroaralkyl.

37. The compound of any one of claims 1-19, wherein R^{Y} is 4-6 membered heterocyclyl.

38. The compound of any one of claims 1-19, wherein R^{Y} is C1-C6 alkyl optionally substituted with —CO₂R^{A} or 5-6 membered heteroaryl optionally substituted with R^{Y1}.

39. The compound of any one of claims 1-19, wherein R^{Y} is C1-C6 alkyl substituted with —CO₂R^{A} or 5-6 membered heteroaryl optionally substituted with R^{Y1}.

40. The compound of any one of claims 1-19, wherein R^{Y} is C1-C6 alkyl substituted with —CO₂R^{A} or 5-6 membered heteroaryl substituted with R^{Y}.

41. The compound of any one of claims 1-19, wherein R^{Y} is C1-C6 alkyl substituted with —CO₂R^{A} or 5-6 membered heteroaryl.

42. The compound of any one of claims 1-19, wherein R^{Y} is C1-C6 alkyl.

43. The compound of any one of claims 38-40, wherein R^{Y1} is —SO₂(C1-C6 alkyl).

44. The compound of any one of claims 38-40, wherein R^{Y1} is C1-C6 alkyl optionally substituted with oxo.

45. The compound of any one of claims 38-40, wherein R^{Y1} is hydroxyl.

46. A compound selected from the group consisting of the compounds in Table A, or a pharmaceutically acceptable salt thereof.

47. A pharmaceutical composition comprising a compound of any one of claims 1-46, or a pharmaceutically acceptable salt thereof, and one or more pharmaceutically acceptable excipients.

48. A method for treating cancer in a subject in need thereof, comprising administering to the subject a therapeutically effective amount of a compound of any one of claims 1-46, or a pharmaceutically acceptable salt thereof, or a pharmaceutical composition of claim 47.

49. A method for treating cancer in a subject in need thereof, the method comprising (a) determining that the cancer is associated with a dysregulation of a PIK3CA gene, a PI3Kα protein, or expression or activity or level of any of the same; and (b) administering to the subject a therapeutically effective amount of a compound of any one of claims 1-46, or a pharmaceutically acceptable salt thereof, or a pharmaceutical composition of claim 47.

50. A method of treating a PI3Kα-associated cancer in a subject, comprising administering to a subject identified or diagnosed as having a PI3Kα-associated cancer a therapeutically effective amount of a compound of any one of claims 1-46 or a pharmaceutically acceptable salt thereof, or a pharmaceutical composition of claim 47.

51. A method for inhibiting mutant PI3Kα activity in a mammalian cell, the method comprising contacting the mammalian cell with an effective amount of a compound of any one of claims 1-46, or a pharmaceutically acceptable salt thereof.
